(19) United States
(12) Patent Application Publication
(10) Pub. No.: US 2025/0340605 A1
(43) Pub. Date: Nov. 6, 2025

(54) GLUCAN BINDING PROTEIN FOR IMPROVING NITROGEN FIXATION IN PLANTS

(71) Applicant: CAMBRIDGE ENTERPRISE LIMITED, Cambridge (GB)

(72) Inventors: Sebastian SCHORNACK, Cambridge (GB); Aleksandr GAVRIN, Cambridge (GB)

(21) Appl. No.: 18/868,448

(22) PCT Filed: May 26, 2023
(86) PCT No.: PCT/GB2023/051409

§ 371 Date: Nov. 22, 2024

(30) Foreign Application Priority Data
May 26, 2022 (GB) 2207774.7

(51) Int. Cl. C07K 14/415; C12N 15/82; C12Q 1/6895

(52) U.S. Cl. CPC C07K 14/415; C12N 15/8261; C12Q 1/6895; C12Q 2600/13; C12Q 2600/156

(57) ABSTRACT

The invention relates to methods for modulating the symbiotic relationship between plants, for example legumes, and nitrogen fixing bacteria in the root nodules. The invention also relates to modified plants, for example gene edited plants that have an altered symbiotic relationship with nitrogen fixing bacteria in the root nodules.

### INTRODUCTION

Nitrogen availability in soil is of critical importance for plant productivity. An increase in the plant available nitrogen in the soil can cause increased plant biomass and higher protein content. However, plants are not able to absorb nitrogen in its natural form and so must rely on the bacterial conversion of nitrogen to ammonia which can then be utilised by plants. Legumes are able to establish symbiotic interactions with nitrogen-fixing rhizobia bacteria resident in the soil. This symbiosis is called root nodule symbiosis. During root nodule symbiosis, bacteria live in the root nodules of the host plants where they convert nitrogen into ammonia which is a plant-available source of nitrogen. Achieving improved nitrogen fixation is the aim of research into symbiosis as this could lead to increased plant biomass, a higher protein content and reduced reliance on nitrogen fertiliser.

The current understanding of root nodule symbiosis is largely restricted to the signalling necessary for its initiation and the development of dedicated organs (Roy et al, 2020). Little is known about the mechanisms controlling the actual fixation and symbiotic efficiency within the root nodules.

The glucan binding protein (GBP) genes are related to the glycosyl hydrolase family 81 genes encoding endo-beta (1,3) glucanases that code for dual domain proteins with glucan-binding and hydrolytic activities towards β-1,3/1,6-glucans (Umemoto et al., 1997; Fliegmann et al., 2004). The GBP gene family is represented by twelve genes in the model legume Medicago truncatula. Several of these genes show a specific upregulation in their transcript levels upon plant or root exposure to fungal and oomycete pathogens indicating the role of GBPs in protecting or defending the plant from pathogen infection.

GBP genes are present in genomes of different plants from bryophytes to seed plants, including legume and non-legume plants. This gene family is particularly expanded in legumes and can comprise several dozens of genes in some polyploid species. Most economically relevant legumes such as pea (Pisum sativum), faba bean (Vicia faba), soybean (Glycine max) and others contain six to twelve GBP genes.

### SUMMARY

The inventors have identified that GBP1 is a negative regulator of the symbiotic relationship between nitrogen-fixing bacteria and legumes in the root nodule. Furthermore, the inventors have found that by mutating plants, for example legumes, to create plants with a loss of function mutation in GBP1 it is possible to modulate the symbiotic relationship between plants, for example legumes, and nitrogen fixing bacteria in the root nodules. Furthermore, the inventors have discovered that by introducing such a mutation into a GBP1 nucleic acid in a plant, the biomass of the plant increases as a consequence of the modulated symbiosis between the plant and the nitrogen fixing bacteria.

As explained above, GBP1 genes have been identified in a number of plant species, including plants from the non-exhaustive list including barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a first embodiment of the invention there is provided a genetically altered plant, for example a legume plant wherein expression of a GBP1 nucleic acid sequence or function of the encoded GBP1 protein is reduced or abolished in said plant.

In a related embodiment of the invention there is provided a genetically altered plant, for example a legume plant, wherein said plant comprises a mutation in the GBP1 nucleic acid sequence, for example selected from SEQ ID NOs: 1 to 48 or a homologue, paralogue, orthologue, or functional variant with at least 70%, 80%, 90% or 95% sequence identity to any one of SEQ ID NOS: 1 to 48.

In a further related embodiment there is provided a genetically altered plant, for example a legume plant, wherein said mutation comprises the deletion and/or insertion and/or replacement of one or more nucleic acids and/or the insertion of a transposon, for example a Tnt-transposon, into a GBP1 nucleic acid sequence, for example a nucleic acid sequence selected from SEQ ID NOs: 1 to 48 or a homologue, paralogue, orthologue, or functional variant with at least 70%, 80%, 90% or 95% sequence identity to any one of SEQ ID NOs: 1 to 48.

In a related embodiment of the invention the genetically altered plant, for example a legume plant, comprises a mutation that reduces or abolishes the promoter activity associated with the expression of GBP1.

In a further related embodiment of the invention there is provided a genetically altered plant, for example a legume plant, wherein said mutation comprises the deletion and/or insertion and/or replacement of one or more nucleic acids and/or nucleic acid regions that make up the promoter region of GBP1.

In a related embodiment of the invention the genetically altered plant may be a legume plant that is selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

In a yet further related embodiment of the invention the mutation is introduced using targeted genome modification.

In a further related embodiment of the invention said mutation is introduced using a rare-cutting endonuclease, for example a TALEN, ZFN or CRISPR/Cas9.

In a related embodiment of the invention the mutation modifies symbiosis with a rhizobacterium in root nodules of the plant.

In a further related embodiment of the invention the mutation modifies symbiosis with a rhizobacterium which increases the nitrogen fixing in root nodules of the plant.

In a related embodiment of the invention the plant is heterozygous or homozygous for the mutation. In a related embodiment of the invention the expression of the GBP1 nucleic acid sequence is reduced or abolished in said plant using RNAi silencing.

Another embodiment of the invention provides a method for modulating nitrogen fixing symbiosis and/or increasing biomass in a plant, for example a legume plant, the method comprising reducing or abolishing the expression of the GBP1 nucleic acid sequence and/or reducing or abolishing the function of the GBP1 protein.

In a related embodiment of the invention the method comprises introducing a mutation in the GBP1 nucleic acid sequence selected from SEQ ID NOs: 1 to 48 or a homologue, paralogue, orthologue, or functional variant with at least 70%, 80%, 90% or 95% sequence identity to any one of SEQ ID NOs: 1 to 48.

In a further related embodiment the method comprises the deletion and/or insertion and/or replacement of one or more nucleic acids and/or the insertion of a transposon into a nucleic acid sequence selected from SEQ ID NOs: 1 to 48. In a related embodiment the transposon is a Tnt-transposon.

In a yet further related embodiment of the invention the method comprises introducing said mutation using targeted genome modification.

In a related embodiment of the invention the method comprises introducing said mutation using a rare-cutting endonuclease, for example a TALEN, ZFN or CRISPR/Cas9.

In a further related embodiment of the invention the method introduces a heterozygous or homozygous mutation into the plant.

In a related embodiment of the invention the method comprises applying a composition to the plant thereby inactivating endogenous GBP1 protein.

In a further embodiment of the invention the composition comprises a mutagenic agent and/or a dsRNA molecule suitable for RNAi silencing.

In a related embodiment of the invention the plant is selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

Another embodiment of the invention provides an isolated mutant GBP1 nucleic acid sequence encoding a mutant GBP1 protein wherein expression of the GBP1 nucleic acid sequence or function of the encoded GBP1 protein is reduced or abolished in a plant.

In a related embodiment of the invention the mutant GBP1 nucleic acid comprises a mutation in the GBP1 nucleic acid sequence selected from SEQ ID NOs: 1 to 48 or a homologue, paralogue, orthologue, or functional variant with about at least 70%, 80%, 90% or 95% sequence identity thereof.

In a further related embodiment of the invention the mutant GBP1 nucleic acid sequence comprises a deletion and/or insertion and/or replacement of one or more nucleic acids and/or a transposon inserted into the nucleic acid sequence selected from SEQ ID NOs: 1 to 48. In a related embodiment the transposon is a Tnt-transposon.

In another related embodiment of the invention the isolated mutant GBP1 nucleic acid sequence is from a plant selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

A further embodiment of the invention provides a vector comprising an isolated nucleic acid of the previous embodiment of the invention.

Another embodiment of the invention provides a host cell comprising a vector of the previous embodiment of the invention.

In another embodiment of the invention a method for producing a plant with modulated nitrogen fixing symbiosis, comprising introducing a mutation into a GBP1 nucleic acid is provided.

In a related embodiment of the invention the method comprised introducing a mutation in the GBP1 nucleic acid of a plant, for example a legume plant, for example into a sequence selected from SEQ ID NOs: 1 to 48 or a homologue, paralogue, orthologue, or functional variant with about a 95% sequence identity thereof.

In a further related embodiment of the invention the method comprises the deletion and/or insertion and/or replacement of one or more nucleic acids and/or insertion of a transposon into the nucleic acid sequence selected from SEQ ID NOs: 1 to 48. In a related embodiment the transposon is a Tnt-transposon.

In another related embodiment of the invention the method comprises introducing the mutation using targeted genome modification.

In a further related embodiment of the invention the method comprised introducing the mutation using a rare-cutting endonuclease, for example a TALEN, ZFN or CRISPR/Cas9.

In a related embodiment of the invention the method is carried out in a plant selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

Another embodiment of the invention provides a method for identifying a plant, for example a legume plant, with altered nitrogen fixing symbiosis compared to a control plant, the method comprising detecting in a population of plants one or more polymorphisms in a GBP1 nucleic acid sequence selected from SEQ ID NOS: 1 to 48 wherein the control plant comprises a GBP1 nucleic acid that encodes a protein having a wild type GBP1 protein.

Another embodiment of the invention provides a detection kit for determining the presence or absence of a polymorphism in the GBP1 protein encoded by a GBP1 nucleic acid sequence in a plant, for example a legume plant.

An embodiment of the invention provides a genetically altered plant, for example a legume plant, wherein expression of a GBP1 nucleic acid sequence or function of the encoded GBP1 protein is reduced or abolished in said plant.

In this related embodiment the invention provides the genetically altered plant, for example a legume plant, wherein said plant comprises a mutation in the GBP1 nucleic acid sequence encoding the GBP1 protein or in a promoter nucleic acid sequence that regulates expression of GBP1.

In this related embodiment the invention provides the genetically altered plant, for example a legume plant, wherein said GBP1 nucleic acid sequence is selected from SEQ ID NOs: 1 to 48 or a homologue, paralogue, orthologue, or functional variant thereof with at least 70%, 80%, 90% or 95% sequence identity to any one of SEQ ID NOs: 1 to 48.

In this related embodiment the invention provides the genetically altered plant, for example a legume plant, wherein said mutation comprises the deletion, insertion, replacement or addition of one or more nucleic acids into the nucleic acid sequence.

In this related embodiment the invention provides the genetically altered plant, for example a legume plant, wherein said mutation comprises the insertion of a transposon into the nucleic acid sequence. In a related embodiment the transposon is a Tnt-transposon.

In this related embodiment the invention provides the genetically altered legume plant wherein said plant is selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2) Medicago, Pea (Pisum sativum, 2), Broad bean (Vicia faba, 1), Clover (Trifolium pratense, 1), Birds treefoil (Lotus japonicus, 1), Lupinus angustifolius, Cowpea (Vigna unguiculata, 3), Common Bean (Phaseolus vulgaris, 3), Soybean (Glycine max, 6), Cajanus cajan, and Chickpea (Cicer arinetum, 1).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

In this related embodiment the invention provides the genetically altered plant, for example a legume plant wherein the mutation is introduced using targeted genome modification.

In this related embodiment the invention provides the genetically altered plant, for example a legume plant wherein said mutation is introduced using a rare-cutting endonuclease, for example a TALEN, ZFN or CRISPR/Cas9.

In this related embodiment the invention provides the genetically altered plant, for example a legume plant, wherein the mutation modifies symbiosis with a rhizobacterium in root nodules of the plant.

In this related embodiment the invention provides the genetically altered plant, for example a legume plant, wherein the mutation modifies symbiosis with a rhizobacterium which increases the nitrogen fixing in root nodules of the plant.

In this related embodiment the invention provides the genetically altered plant, for example a legume plant, wherein the plant is homozygous for the mutation.

In this related embodiment the invention provides the genetically altered plant, for example a legume plant, wherein the expression of the GBP1 nucleic acid sequence is reduced or abolished in said plant using RNAi silencing.

An embodiment of the invention provides a method for modulating nitrogen fixing symbiosis in a plant, for example a legume plant, and/or increasing plant biomass, the method comprising reducing or abolishing the expression of a GBP1 nucleic acid sequence encoding a GBP1 protein and/or reducing or abolishing the function of the GBP1 protein or a homologue, paralogue, orthologue, or functional variant thereof.

In a related embodiment the invention provides the method wherein the method comprises introducing a mutation in the GBP1 nucleic acid sequence encoding the GBP1 protein or in a promoter nucleic acid sequence that regulates expression of GBP1.

In a related embodiment the invention provides the method wherein said GBP1 nucleic acid sequence selected from SEQ ID NOs: 1 to 48 or a homologue, paralogue, orthologue, or functional variant with at least 70%, 80%, 90% or 95% sequence identity to any one of SEQ ID NOs: 1 to 48.

In a related embodiment the invention provides the method wherein said mutation comprises the deletion, insertion, replacement and/or addition of one or more nucleic acids into the nucleic acid sequence.

In a related embodiment the invention provides the method wherein said mutation comprises the insertion of a transposon into the nucleic acid sequence selected from SEQ ID NOs: 1 to 48. In a related embodiment the transposon is a Tnt-transposon.

In a related embodiment the invention provides the method wherein the method comprises introducing said mutation using targeted genome modification.

In a related embodiment the invention provides the method wherein the method comprises introducing said mutation using a rare-cutting endonuclease, for example a TALEN, ZFN or CRISPR/Cas9.

In a related embodiment the invention provides the method wherein the method introduces a homozygous mutation into the plant.

In a related embodiment the invention provides the method wherein the method comprises applying a mutagenic composition to the plant.

In a related embodiment the invention provides the method wherein the method comprises introducing into said plant a dsRNA molecule suitable for RNAi silencing.

In a related embodiment the invention provides the method wherein said plant is selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2) Medicago, Pea (Pisum sativum, 2), Broad bean (Vicia faba, 1), Clover (Trifolium pratense, 1), Birds treefoil (Lotus japonicus, 1), Lupinus angustifolius, Cowpea (Vigna unguiculata, 3), Common Bean (Phaseolus vulgaris, 3), Soybean (Glycine max, 6), Cajanus cajan, and Chickpea (Cicer arinetum, 1).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

An embodiment of the invention provides an isolated mutant GBP1 nucleic acid sequence encoding a mutant GBP1 protein wherein expression of the GBP1 nucleic acid sequence or function of the encoded GBP1 protein is reduced or abolished in a plant.

In a related embodiment the invention provides the isolated mutant GBP1 nucleic acid sequence wherein the mutant GBP1 nucleic acid comprises a mutation in the GBP1 nucleic acid sequence selected from SEQ ID NOs: 1 to 48 or a homologue, paralogue, orthologue, or functional variant with at least 70%, 80%, 90% or 95% sequence identity thereto.

In a related embodiment the invention provides the isolated mutant of GBP1 nucleic acid sequence wherein the mutant GBP1 nucleic acid sequence comprises a deletion, insertion, addition and/or replacement of one or more nucleic acids and/or a transposon inserted into the nucleic acid sequence selected from SEQ ID NOs: 1 to 48. In a related embodiment the transposon is a Tnt-transposon.

In a related embodiment the invention provides the isolated mutant of GBP1 nucleic acid sequence wherein the mutant GBP1 nucleic acid sequence is from a plant selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2) Medicago, Pea (Pisum sativum, 2), Broad bean (Vicia faba, 1), Clover (Trifolium pratense, 1), Birds treefoil (Lotus japonicus, 1), Lupinus angustifolius, Cowpea (Vigna unguiculata, 3), Common Bean (Phaseolus vulgaris, 3), Soybean (Glycine max, 6), Cajanus cajan, and Chickpea (Cicer arinetum, 1).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

An embodiment of the invention provides a vector comprising an isolated nucleic acid of the previous embodiment.

An embodiment of the invention provides a host cell comprising a vector of the previous embodiment.

An embodiment of the invention provides a method for producing a plant with modulated nitrogen fixing symbiosis, comprising introducing a mutation into a GBP1 nucleic acid or in a promoter nucleic acid sequence that regulates expression of GBP1.

In a related embodiment the invention provides the method for producing a plant with modulated nitrogen fixing symbiosis, comprising introducing a mutation in the GBP1 nucleic acid sequence selected from SEQ ID NOS: 1 to 48 or a homologue, paralogue, orthologue, or functional variant with at least 70%, 80%, 90% or 95% sequence identity thereto.

In a related embodiment the invention provides the method for producing a plant with modulated nitrogen fixing symbiosis, comprising the wherein said mutation comprises the deletion, insertion, replacement and/or addition of one or more nucleic acids into the nucleic acid sequence and/or insertion of a transposon into the nucleic acid sequence selected from SEQ ID NOs: 1 to 201 to 48. In a related embodiment the transposon is a Tnt-transposon.

In a related embodiment the invention provides the method for producing a plant with modulated nitrogen fixing symbiosis, comprising introducing the mutation using targeted genome modification.

In a related embodiment the invention provides the method for producing a plant with modulated nitrogen fixing symbiosis, comprising introducing the mutation using a rare-cutting endonuclease, for example a TALEN, ZFN or CRISPR/Cas9.

In a related embodiment the invention provides the method for producing a plant with modulated nitrogen fixing symbiosis, wherein the method is carried out in a plant selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2) Medicago, Pea (Pisum sativum, 2), Broad bean (Vicia faba, 1), Clover (Trifolium pratense, 1), Birds treefoil (Lotus japonicus, 1), Lupinus angustifolius, Cowpea (Vigna unguiculata, 3), Common Bean (Phaseolus vulgaris, 3), Soybean (Glycine max, 6), Cajanus cajan, and Chickpea (Cicer arinetum, 1).

An embodiment of the invention provides a method for identifying a plant with altered nitrogen fixing symbiosis compared to a control plant, the method comprising detecting in a population of plants one or more polymorphisms in a GBP1 nucleic acid sequence.

In a related embodiment the invention provides the method or identifying a plant with altered nitrogen fixing symbiosis compared to a control plant, wherein the GBP1 nucleic acid sequence is selected from SEQ ID NOs: 1 to 48 or a homologue, paralogue, orthologue, or functional variant with about at least 70%, 80%, 90% or 95% sequence identity thereto wherein the control plant comprises a GBP1 nucleic acid that encodes a protein having a wild type GBP1 protein.

An embodiment of the invention provides a detection kit for determining the presence or absence of a polymorphism in aGBP1 nucleic acid sequence in a plant, for example a legume plant.

### FIGURES

The invention is further described in the following non-limiting figures:

FIG. 1: Graphs showing GBP1 expression is strongly upregulated in root tissues during nitrogen fixing symbiosis with Sinorhizobium meliloti (A), and unaltered upon infection with Rhizoctonia solani (B), Botrytis cinerea (C), Phytophthora palmivora (D) or laminarin treatment (E).

FIG. 2: Microscopy images showing GBP1 expression during root infection by rhizobia S. meliloti and in the developed root nodule. The top “Overlay+brightfield” image shows the infection thread containing the bacteria has passed through the root hair and has started to enter the nodule primordium. The lower “Overlay+brightfield” image shows a fully developed root nodule where GBP1 expression is limited to the zones where bacteria release into plant cells and develop into bacteroides (nitrogen fixing organelle-like intracellular structures).

FIG. 3: Two graphs that show transcriptional activation of GBP1 in response to S. meliloti infection in wild type Medicago and Medicago mutants with either dysfunctional transcription factor NIN (NODULE INCEPTION), Nod-factor receptor NFP (Nod factor perception) (A) or chitin receptor LYK9 (B). The graphs show that GBP1 activation in response to Rhizobacterial infection is dependent on the Common Symbiosis Signalling Pathway.

FIG. 4: Schematic representation of transposon insertions in GBP1 and their position relative to the translation start site. gbp1-1 and gbp1-3 lines have upregulated levels of GBP1 transcript, gbp1-4 is a knockout line and gbp1-5 has a disrupted open reading frame resulting in truncated non-functional GBP1 proteins.

FIG. 5: Photographs of root nodules formed by each Medicago line (1-1, 1-3, 1-4 and 1-5).

FIG. 6: Microscopic images of wild type GBP1-4 and the gbp1-4 knockout line dissected root nodules (A) and nodule cells (B) colonised by S. meliloti expressing GFP under NifH promoter Quantification of GFP fluorescence (C) shows an increase in NifH expression in nodules of the gbp1-4 Medicago line compared to wildtype. Quantification of bacteroid volume (D) shows that gbp1-4 line nodules contains smaller bacteroids.

FIG. 7: Graphs that show the relative expression of GBP1 gene (A, C) and nodulation quantification (B, D) in wildtype GBP1-1 or GBP1-4 and the gbp1-1 or gbp1-4 mutant lines cultivated in mock (non-inoculated) conditions or in the presence of a symbiotic rhizobacterium S. meliloti

FIG. 8: Graphs that show the results of nodule nitrogenase activity (A, C) and level of shoot biomass accumulation (B, D) in wildtype GBP1-1 or GBP1-4 and the gbp1-1 or gbp1-4 mutant lines cultivated with symbiotic bacteria.

FIG. 9: Two graphs that show the number of nodules present on the roots of Medicago plants modified to display constitutive ectopic expression of GBP1 under the control of the Ubiquitin promoter (pUbq: GBP1) compared to control Medicago plants expressing an empty vector (pUbq: EV) at 10 days post inoculation (dpi) (A) and at 17 dpi (B).

FIG. 10: Photographs of the root system of a pUbq: GBP1 expressing Medicago plant and the control pUbq: EV Medicago plant with the root nodules displaying as fluorescent.

FIG. 11: Two graphs showing the relative expression of Pea (Pisum sativum) GBP1 (A) and GBP2 (B) in root nodules when Pea plants are cultivated in the presence of the symbiotic bacterium Rhizobium leguminosarum (Rlv3841) compared to non-inoculated plants (mock).

FIG. 12: Graphs showing the relative expression of Broad Bean (Vicia Fabia) GBP1 in root nodules when Broad Bean plants are cultivated in the presence of the symbiotic bacterium Rhizobium leguminosarum (Rlv3841) compared to non-inoculated plants (mock).

FIG. 13: Brightfield and DsRed fluorescent images of the Pea roots expressing empty vector control pUbq: EV (A) and pUbq: PsGBP1 (Psat3g201680.1) (B).

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

All aspects and embodiments of the invention relate to legume and non-legume plants. In a preferred embodiment, all aspects and embodiments of the invention relate to legume and non-legume plants.

In a first aspect, a genetically altered plant, for example a legume plant, is provided wherein the expression of a GBP1 nucleic acid sequence or function of the encoded GBP1 protein is reduced or abolished in said plant.

In one embodiment, the expression of the GBP1 nucleic acid can be reduced or abolished by manipulating the promoter sequence of the GBP1 gene, that is the regulatory sequence or by manipulating the coding sequence of the gene.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), naturally occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA. The terms “peptide”, “polypeptide” and “protein” are used interchangeably herein and refer to amino acids in a polymeric form of any length, linked together by peptide bonds. The term “allele” designates any of one or more alternative forms of a gene at a particular locus. Heterozygous alleles are two different alleles at the same locus. Homozygous alleles are two identical alleles at a particular locus. A wild type (wt) allele is a naturally occurring allele without a modification at the target locus.

The terms “increase”, “improve” or “enhance” are interchangeable. Yield or biomass for example can be increased by at least 3%, 4%, 5%, 6%, 7%, 8%, 9% or 10%, preferably at least 15% or 20%, more preferably 25%, 30%, 35%, 40% or 50% or more in comparison to a control plant. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. Thus, according to the invention, yield comprises one or more of and can be measured by assessing one or more of: increased seed yield per plant, increased seed filling rate, increased number of filled seeds, increased harvest index, increased number of seed capsules and/or pods, increased seed size, increased growth or increased branching, for example inflorescences with more branches. Yield is increased relative to control plants. For the purposes of the invention, a “genetically altered plant” or “mutant plant” is a plant that has been genetically altered compared to a control plant.

A control plant as used herein is a plant, e.g. of the same species, which has not been modified according to the methods of the invention. Accordingly, the control plant does not have a mutant GBP1 nucleic acid sequence as described herein. In one embodiment, the control plant is a wild type plant that does not have a loss of function mutation in a GBP1 nucleic acid, for example does not have a modification at the nucleic acid encoding the GBP1 protein. In another embodiment, the control plant is a plant that does not have a mutant GBP1 nucleic acid sequence as described here, but is otherwise modified. The control plant is typically of the same plant species, preferably the same ecotype or the same or similar genetic background as the plant to be assessed.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid of interest. The term “plant” also encompasses plant cells, suspension cultures, protoplasts, callus tissue, embryos, meristematic regions, gametophytes, sporophytes, pollen and microspores, again wherein each of the aforementioned comprises the gene/nucleic acid of interest.

Recently, genome editing techniques have emerged as alternative methods to conventional mutagenesis methods (such as physical and chemical mutagenesis) or methods using the expression of transgenes in plants to produce mutant plants with improved phenotypes that are important in agriculture. These techniques employ sequence-specific nucleases (SSNs) including zinc finger nucleases (ZFNs), transcription activator-like effector nucleases (TALENs), and the RNA-guided nuclease Cas9 (CRISPR/Cas9), which generate targeted DNA double-strand breaks (DSBs), which are then repaired mainly by either error-prone non-homologous end joining (NHEJ) or high-fidelity homologous recombination (HR). As explained in detail herein, mutations according to the invention can be introduced into plants using targeted genome modification based on such editing techniques.

For the purposes of certain other embodiments of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either (a) the nucleic acid sequences encoding proteins useful in the methods of the invention, or (b) genetic control sequence(s) which is operably linked with the nucleic acid sequence according to the invention, for example a promoter, or (c) a) and b) are not located in their natural genetic environment or have been modified by recombinant methods.

The term “vector” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression of the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self-replicating extrachromosomal vectors or vectors which integrate into a host genome. Preferred vectors are those capable of autonomous replication and/or expression of nucleic acids to which they are linked. Vectors capable of directing the expression of genes to which they are operatively linked are referred to herein as “expression vectors”.

The term “regulatory sequences” is used interchangeably with “regulatory elements” herein refers to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequences to which they are operatively linked. Regulatory sequences often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

Enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

As used herein, a “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest. In one embodiment, the promoter is a constitutive promoter. A “constitutive promoter” refers to a promoter that is transcriptionally active during most, but not necessarily all, phases of growth and development and under most environmental conditions, in at least one cell, tissue or organ. Examples of constitutive promoters include but are not limited to actin, HMGP, CaMV19S, GOS2, rice cyclophilin, maize H3 histone, alfalfa H3 histone, 34S FMV, rubisco small subunit, OCS, SAD1, SAD2, nos, V-ATPase, super promoter, G-box proteins, Arabidopsis Ubiquitin promoters and synthetic promoters. In another aspect of the invention there is provided a vector comprising the nucleic acid sequence described above.

Plants of the invention have modified root phenotype, i.e. modified root growth compared to a control plant. The term modified root growth refers to a root growth with an improved nitrogen fixing symbiosis compared to the nitrogen fixing symbiosis found in a control plant. The root nitrogen fixing symbiosis is defined as the amount of nitrogen fixed per unit root mass of each root, and can be quantified to provide a synthetic indicator of the proportion of the total number of roots that have an improved nitrogen fixing symbiosis. Plants of the invention have a significantly increased root nitrogen fixing symbiosis than control plants. This can be tested in various ways. For e.g. legume plants, root nitrogen fixing symbiosis can be simply measured by measuring the rate of acetylene reduction of each plant. As explained herein, increased root nitrogen fixing symbiosis can result in increased yield.

Thus, as used herein, the term GBP1 nucleic acid sequence or GBP1 gene refers to any nucleic acid sequence, e.g. a gene, that encodes a GBP1 protein. The GBP1 nucleic acid sequence may be from a legume plant or non-legume plant. For example, the GBP1 nucleic acid sequence may comprise or consist of any of SEQ ID NOs: 1 to 48, a functional variant, homolog, paralog or ortholog thereof as defined herein. For example, the encoded protein comprises or consists of SEQ ID NOs: 21 to 41. Thus, in one embodiment, the term GBP1 nucleic acid sequence or GBP1 gene refers to a sequence or GBP1 gene refers to a nucleic acid sequence (SEQ ID NOS: 1 to 48), e.g. a gene, that encodes a protein characterised by SEQ ID NOs: 21 to 41 and this can be a homologue, paralogue, orthologue or functional variant of GBP1.

The term “functional variant of a nucleic acid sequence” as used herein with reference to SEQ ID NO: 1 to 48 refers to a variant gene sequence or part of the gene sequence which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. The term “functional variant of an amino acid sequence” as used herein, e.g. with reference to SEQ ID NO: 49 to 96 refers to a variant protein sequence.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence; e.g. SEQ ID NO. 1 or a homologue or orthologue thereof, e.g. SEQ ID NO. 2-96.

The term orthologue as used herein designates an GBP1 gene orthologue from other plant species. A homolog or orthologue may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequence presented by SEQ ID NO: 1 or to the amino acid sequence shown in SEQ ID NO: 48 or to a nucleic acid sequence presented by SEQ ID NO: 2-48 or to an amino acid sequence shown in 49-96. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, e.g. 90%, 91%, 92%, 93%, 94%, 95%, 96%, 97%, 98%, or at least 99%. Functional variants of GBP1 homologs/orthologues as defined above are also within the scope of the invention. Examples are orthologues from crop species as listed below.

In one embodiment, the GBP1 nucleic acid sequence is selected from SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% thereto. In one embodiment, the GBP1 amino acid sequence is selected from SEQ ID NO. 49, 50, 51, 52, 53, 54, 55, 56, 57, 58, 59, 60, 61, 62, 63, 64, 65, 67, 68, 69, 70, 71, 72, 73, 74, 75, 76, 77, 78, 79, 80, 81, 82, 83, 84, 85, 86, 88, 89, 90, 91, 92, 93, 94, 95, 96, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% thereto.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologs/orthologues can be identified by sequence comparisons and identifications of conserved domains. There are predictors in the art that can be used to identify such sequences. The function of the homologue can be identified as described herein and a skilled person would thus be able to confirm the function, for example when not expressed in a plant.

An embodiment of the present invention provides a method for identifying a plant, e.g. a legume plant, with altered nitrogen fixing symbiosis compared to a control plant, the method comprising detecting in a population of plants with one or more polymorphisms in a GBP1 nucleic acid sequence selected from SEQ ID NOs: 1 to 48 wherein the control plant comprises a GBP1 nucleic acid that encodes a wild type GBP1 protein.

In a related embodiment of the present invention the GBP1 nucleic acid sequence is a homologue, paralogue or orthologue of the GBP1 nucleic acid sequences of SEQ ID NOs: 1 to 48.

In further related embodiments of the present invention the homologue, paralogue or orthologue shares at least 80%, 90% or 95% identity with any of the sequences of SEQ ID NOs: 1 to 48.

In a further embodiment of the present invention the method for identifying a plant, for example a legume plant, with altered nitrogen fixing symbiosis compared to a control plant additionally comprises measuring the acetylene reduction of a wild type plant and the population of plants in which the altered nitrogen fixing symbiosis is to be detected.

Thus, the nucleotide sequences of the invention and described herein can also be used to isolate corresponding sequences from other organisms, particularly other plants, for example crop plants, including non-legume plants. In this manner, methods such as PCR, hybridization, and the like can be used to identify such sequences based on their sequence homology to the sequences described herein. Topology of the sequences and the characteristic domain structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g. at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na+ ion, typically about 0.01 to 1.0 M Na+ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further embodiment, a variant as used herein can comprise a nucleic acid sequence encoding a GBP1 polypeptide as defined herein that is capable of hybridising under stringent conditions as defined herein to a nucleic acid sequence as defined in any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48. The inventors have shown that GBP1 expression is upregulated during nitrogen fixing symbiosis. Thus, in a further related embodiment, the nucleic acid sequence encoding GBP1 can be further identified by determining the upregulation of expression of the nucleic acid sequence during nitrogen fixing symbiosis.

In one embodiment, the orthologue of the GBP1 nucleic acid sequence as shown in SEQ ID NO. 1 is a GBP1 nucleic acid of a legume plant. Thus, the genetically altered plant may be a plant, for example a legume plant with a mutation in an endogenous GBP1 nucleic acid sequence encoding a mutant GBP1 protein.

In one embodiment the legume plant may be any of barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

In one embodiment, the plant is not a Medicago plant with a transposon insertion in the GBP1 nucleic acid sequence.

In one embodiment, the plant is heterozygous or homozygous for the mutation.

The invention also extends to harvestable parts of a genetically altered plant of the invention as described above such as, but not limited to seeds, leaves, flowers, stems and roots. The invention furthermore relates to products derived, preferably directly derived, from a harvestable part of such a plant, such as dry pellets or powders, oil, fat and fatty acids, flour, starch or proteins. The invention also relates to food products and food supplements comprising the plant of the invention or parts thereof. In one aspect, the invention relates to a seed of a mutant plant of the invention.

In another embodiment, the present invention provides a regenerable mutant plant as described herein and cells for use in tissue culture. The tissue culture will preferably be capable of regenerating plants having essentially all of the physiological and morphological characteristics of the foregoing mutant plant, and of regenerating plants having substantially the same genotype. Preferably, the regenerable cells in such tissue cultures will be callus, protoplasts, meristematic cells, cotyledons, hypocotyl, leaves, pollen, embryos, roots, root tips, anthers, pistils, shoots, stems, petioles, flowers, and seeds. Still further, the present invention provides plants regenerated from the tissue cultures of the invention.

In one embodiment, the genetically altered plant, for example a legume plant, is a plant that has been altered using a mutagenesis method, such as any of the mutagenesis methods described herein. In one embodiment, the mutagenesis method is targeted genome modification (genome editing) as further explained herein. Such plants have an altered root phenotype as described herein. Therefore, in this example, the phenotype is conferred by the presence of an altered plant genome, i.e., a mutated endogenous GBP1 gene. In one embodiment, the GBP1 gene sequence is specifically targeted using targeted genome modification. Thus, the presence of a mutated GBP1 gene sequence is not conferred by the presence of transgenes expressed in the plant. In other words, the genetically altered plant can be described as transgene-free. Gene editing techniques that can be used to generate the plant are further described below.

In one embodiment, the genetically altered plant is not exclusively obtained by means of an essentially biological process. For example, the mutation has been introduced in the GBP1 nucleic acid sequence using targeted genome modification, for example with a construct as described herein.

In the aspects and embodiments described herein, the GBP1 protein may have hydrolylase activity, for example endo-β-1,3-glucanase activity.

Methods for Modulating Plant Traits/Producing Plants with Modulated Traits

A skilled person would appreciate that modulating nitrogen fixing symbiosis can be achieved by different means that include modulating the GBP1 signal, gene expression, or function of GBP1 of the GBP1 protein. This may include inhibiting GBP1 activity, GBP1 signaling, downregulating GBP1 protein level, downregulating GBP1 expression or knockdown of GBP1 gene expression. For example, GBP signal reduction, elimination, or inhibition can be achieved by small molecule inhibitors, RNAis, dsRNA, shRNA, siRNA, miRNA, or ASOs, CRISPR Cas9, or analogous technologies. In one embodiment, such modification reduces or prevents hydrolase activity, for example endo-β-1,3-glucanase expression or activity directly or indirectly by inhibiting production or activity upstream or downstream.

Thus, in one embodiment, the invention relates to a method for modulating nitrogen fixing symbiosis in a plant, for example a legume plant, the method comprising reducing or abolishing the expression of the GBP1 nucleic acid sequence or a homologue, paralogue, orthologue, or functional variant thereof and/or reducing or abolishing the function of the GBP1 protein or a homologue, paralogue, orthologue, or functional variant thereof.

In a further embodiment of the invention the method comprises introducing a mutation in the GBP1 nucleic acid sequence, for example a nucleic acid selected from SEQ ID NOs: 1 to 48 or a homologue, paralogue, orthologue, or functional variant with at least 70%, 80%, 90% or 95% sequence identity to any one of SEQ ID NOs: 1 to 48.

In a yet further embodiment of the invention the method comprises the deletion and/or insertion and/or replacement of one or more nucleic acids and/or the insertion of a transposon into a GBP1 nucleic acid sequence, for example a sequence selected from SEQ ID NOs: 1 to 48. In a related embodiment the transposon is a Tnt-transposon.

In one embodiment, the method does not relate to a Medicago plant with a transposon insertion in the GBP1 nucleic acid sequence.

In another embodiment of the invention the method comprises introducing said mutation using targeted genome modification, (e.g. genome editing).

In a related embodiment of the invention the method comprises introducing said mutation using a rare-cutting endonuclease, for example a TALEN, ZFN or CRISPR/Cas9.

In a further related embodiment of the invention the method introduces a heterozygous or homozygous mutation into the plant.

In a related embodiment of the invention the method comprises applying a composition to the plant thereby inactivating endogenous GBP1 protein.

In a further related embodiment of the invention the composition comprises a mutagenic agent and/or a dsRNA molecule suitable for RNAi silencing.

In a related embodiment of the invention said plant is selected from barrel medic (Medicago truncatula, 1), alfalfa (Medicago sativa, 8), pea (Pisum sativum, 2), broad bean (Vicia faba, 1), red clover (Trifolium pratense, 1), white clover (Trifolium repens, 2), subterranean clover (Trifolium subterraneum, 1), birds treefoil (Lotus japonicus, 1), blue lupin (Lupinus angustifolius, 2), white lupin (Lupinus albus, 2) Cowpea (Vigna unguiculata, 3), Common Bean (Phaseolus vulgaris, 3), Soybean (Glycine max, 6), pigeon pea (Cajanus cajan, 2), lima bean (Phaseolus lunatus, 5), tepary bean (Phaseolus acutifolius, 6), and chickpea (Cicer arinetum, 2).

In a yet further embodiment, the plant may be a non-legume plant, for example Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

### Targeted Genome Modification Using Gene Editing

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customizable DNA binding proteins can be used: meganucleases derived from microbial mobile genetic elements, ZF nucleases based on eukaryotic transcription factors, rare-cutting endonucleases/sequence specific endonucleases (SSN), for example TALENs, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate their nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type Ill secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Customized plasmids can be used with the Golden Gate cloning method to assemble multiple DNA fragments. The Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method that can be used according to the various aspects of the invention is CRISPR. The use of this technology in genome editing is well described in the art, for example in U.S. Pat. No. 8,697,359. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage. Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer).

The Type II CRISPR is one of the most well characterized systems and carries out targeted DNA double-strand breaks in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the type II CRISPR-Cas system, and a large monomeric DNA nuclease guided to a DNA target sequence adjacent to the PAM sequence motif by a complex of two noncoding RNAs: CRIPSR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with a guide RNA (gRNA) also called single guide RNA (sgRNA) can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

Synthetic CRISPR systems typically consist of two components, the gRNA and a non-specific CRISPR-associated endonuclease and can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA. The genomic target can be any 20 nucleotide DNAsequence, provided that the target is present immediately upstream of a PAM sequence. The PAM sequence is of outstanding importance for target binding and the exact sequence is dependent upon the species of Cas9.

The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337:816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

Once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

CRISPR/Cas9 and likewise CRISPR/Cpf1 and other CRISPR systems are highly specific when gRNAs are designed correctly, but especially specificity is still a major concern, particularly for clinical uses based on the CRISPR technology. The specificity of the CRISPR system is determined in large part by how specific the gRNA targeting sequence is for the genomic target compared to the rest of the genome. The sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Thus, as used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

sgRNAs suitable for use in the methods of the invention are described below.

As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be an RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also contemplated. The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeably herein to refer to a target sequence that is endogenous or native to the genome of a plant and is at the endogenous or native position of that target sequence in the genome of the plant.

The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

In one embodiment, the Cas endonuclease gene is a Cas9 endonuclease, such as but not limited to, Cas9 genes listed in WO2007/025097 incorporated herein by reference. In another embodiment, the Cas endonuclease gene is plant, maize or soybean optimized Cas9 endonuclease.

In one embodiment, the Cas endonuclease gene is a plant codon optimized Streptococcus pyogenes Cas9 gene that can recognize any genomic sequence of the form N (12-30) NGG can in principle be targeted.

In one embodiment, the Cas endonuclease is introduced directly into a cell by any method known in the art, for example, but not limited to transient introduction methods, transfection and/or topical application.

Cas9 expression plasmids for use in the methods of the invention can be constructed as described in the art and as described in the examples.

In one embodiment, targeted genome modification according to the various aspects of the invention comprises the use of a rare-cutting endonuclease, for example a TALEN, ZFN or CRISPR/Cas; e.g. CRISPR/Cas9. Rare-cutting endonucleases/sequence specific endonucleases are naturally or engineered proteins having endonuclease activity and are target specific. These bind to nucleic acid target sequences which have a recognition sequence typically 12-40 bp in length. In one embodiment, the SSN is selected from a TALEN. In another embodiment, the SSN is selected from CRISPR/Cas9. This is described in more detail below.

In one embodiment, the step of introducing a mutation comprises contacting a population of plant cells with DNA binding protein targeted to an endogenous GBP1 gene sequence, for example selected from the exemplary sequences listed herein. In one embodiment, the method comprises contacting a population of plant cells with one or more rare-cutting endonucleases; e.g. ZFN, TALEN, or CRISPR/Cas9, targeted to an endogenous GBP1 gene sequence.

The method may further comprise the steps of selecting, from said population, a cell in which a GBP1 gene sequence has been modified and regenerating said selected plant cell into a plant.

In an embodiment, the method comprises the use of CRISPR/Cas9. In this embodiment, the method therefore comprises introducing and co-expressing in a plant Cas9 and sgRNA targeted to a GBP1 gene sequence and screening for induced targeted mutations in a GBP1 nucleic gene. The method may also comprise the further step of regenerating a plant and selecting or choosing a plant with an altered root phenotype, e.g. having a steeper root angle.

Cas9 and sgRNA may be comprised in a single or two expression vectors. The target sequence is a GBP1 nucleic acid sequence as shown herein.

In one embodiment, screening for CRISPR-induced targeted mutations in a GBP1 gene comprises obtaining a DNA sample from a transformed plant and carrying out DNA amplification and optionally restriction enzyme digestion to detect a mutation in a GBP1 gene.

In one embodiment, the restriction enzyme is mismatch-sensitive T7 endonuclease. T7E1 is an enzyme that is specific to heteroduplex DNA caused by genome editing.

PCR fragments amplified from the transformed plants are then assessed using a gel electrophoresis assay based assay. In a further step, the presence of the mutation may be confirmed by sequencing the GBP1 gene. Genomic DNA (i.e. wt and mutant) can be prepared from each sample, and DNA fragments encompassing each target site are amplified by PCR. The PCR products are digested by restriction enzymes as the target locus includes a restriction enzyme site. The restriction enzyme site is destroyed by CRISPR- or TALEN-induced mutations by NHEJ or HR, thus the mutant amplicons are resistant to restriction enzyme digestion, and result in uncleaved bands. Alternatively, the PCR products are digested by T7E1 (cleaved DNA produced by T7E1 enzyme that is specific to heteroduplex DNA caused by genome editing) and visualized by agarose gel electrophoresis. In a further step, they are sequenced.

In one embodiment, the method uses the sgRNA (and template, synthetic single-strand DNA oligonucleotides (ssDNA oligos) or donor DNA) constructs defined in detail below to introduce a targeted SNP or mutation, in particular one of the substitutions described herein into a GRF gene and/or promoter. The introduction of a template DNA strand, following a sgRNA-mediated snip in the double-stranded DNA, can be used to produce a specific targeted mutation (i.e. a SNP) in the gene using homology directed repair. Synthetic single-strand DNA oligonucleotides (ssDNA oligos) or DNA plasmid donor templates can be used for precise genomic modification with the homology-directed repair (HDR) pathway. Homologous recombination is the exchange of DNA sequence information through the use of sequence homology. Homology-directed repair (HDR) is a process of homologous recombination where a DNA template is used to provide the homology necessary for precise repair of a double-strand break (DSB). CRISPR guide RNAs program the Cas9 nuclease to cut genomic DNA at a specific location. Once the double-strand break (DSB) occurs, the mammalian cell utilizes endogenous mechanisms to repair the DSB. In the presence of a donor DNA, either a ssDNA oligo or a plasmid donor, the DSB can be repaired precisely using HDR resulting in a desired genomic alteration (insertion, removal, or replacement).

Single-strand DNA donor oligos are delivered into a cell to insert or change short sequences (SNPs, amino acid substitutions, epitope tags, etc.) of DNA in the endogenous genomic target region.

A “donor sequence” is a nucleic acid sequence that contains all the necessary elements to introduce the specific substitution into a target sequence, preferably using homology-directed repair (HDR). In one embodiment, the donor sequence comprises a repair template sequence for introduction of at least one SNP. Preferably the repair template sequence is flanked by at least one, preferably a left and right arm, more preferably around 100 bp each that are identical to the target sequence. More preferably the arm or arms are further flanked by two gRNA target sequences that comprise PAM motifs so that the donor sequence can be released by Cas9/gRNAs. Donor DNA has been used to enhance homology directed genome editing (e.g. Richardson et al, Enhancing homology-directed genome editing by catalytically active and inactive CRISPR-Cas9 using asymmetric donor DNA, Nature Biotechnology, 2016 March; 34(3): 339-44).

The methods above use plant transformation to introduce an expression vector comprising a sequence-specific nucleases into a plant to target a GBP1 nucleic acid sequence. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art. The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle bombardment as described in the examples, transformation using viruses or pollen and microinjection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

The sequence-specific nuclease is preferably introduced into a plant as part of an expression vector. The vector may contain one or more replication systems which allow it to replicate in host cells. Self-replicating vectors include plasmids, cosmids and virus vectors. Alternatively, the vector may be an integrating vector which allows the integration into the host cell's chromosome of the DNA sequence. The vector desirably also has unique restriction sites for the insertion of DNA sequences. If a vector does not have unique restriction sites it may be modified to introduce or eliminate restriction sites to make it more suitable for further manipulation. Vectors suitable for use in expressing the nucleic acids, are known to the skilled person and a non-limiting example is pYP010. The nucleic acid is inserted into the vector such that it is operably linked to a suitable plant active promoter. Suitable plant active promoters for use with the nucleic acids include, but are not limited to CaMV35S, wheat U6, Arabidopsis or maize ubiquitin promoters.

### Conventional Mutagenesis Methods

As an alternative to the gene editing methods described above, more conventional mutagenesis methods can be used in the methods of the invention to introduce at least one mutation into a GBP1 gene sequence, for example the SEQ ID NO. 1 to 48. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. In one embodiment, insertional mutagenesis is used, for example using T-DNA mutagenesis (which inserts pieces of the T-DNA from the Agrobacterium tumefaciens T-Plasmid into DNA causing either loss of gene function or loss of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 1 1, 2283-2290 December 1999).

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the GBP1 nucleic acid sequence in a wild type plant and phenotypic analysis is also carried out. In another embodiment, mutagenesis is physical mutagenesis, such as application of ultraviolet radiation, X-rays, gamma rays, fast or thermal neutrons or protons. The targeted population can then be screened to identify a GBP1 loss of function mutant.

In another embodiment of the various aspects of the invention, the method comprises applying to the plant a mutagenic composition, thus mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1 ‘EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz (a) anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify a GBP1 gene with a mutation resulting from the mutagenesis.

In another embodiment, the method used to create and analyse mutations is targeting induced local lesions in genomes (TILLING), reviewed in Henikoff et al, Plant Physiol. 2004 June; 135(2): 630-636. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the GBP1 target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the GBP1 nucleic acid sequence may be utilized to amplify the GBP1 nucleic acid sequence within the pooled DNA sample. Preferably, the primer is designed to amplify the regions of the GBP1 gene where useful mutations are most likely to arise, specifically in the areas of the GBP1 gene that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is a molecular technique that is similar to TILLING, except that its objective is to uncover natural variation in a given population as opposed to induced mutations.

Rapid high-throughput screening procedures thus allow the analysis of amplification products for identifying a dominant loss of function mutant as compared to a corresponding non-mutagenised wild type plant. Once a mutation is identified in a gene of interest, the seeds of the M2 plant carrying that mutation are grown into adult M3 plants and screened for the phenotypic characteristics associated with the target gene GBP1. Loss of function mutants with improved yield and/or improved nitrogen fixing symbiosis, i.e. increased biomass and/or increased acetylene reduction in an acetylene reduction assay, compared to a control can thus be identified.

Plants obtained or obtainable by any of the methods described above method, such as plants, including legume plants, which carry a loss of function mutation in the endogenous GBP1 gene, are also within the scope of the invention.

### RNA Interference

RNA interference (RNAi) is a biological process in which RNA molecules are involved in sequence-specific suppression of gene expression by double-stranded RNA, through translational or transcriptional repression. Two types of small RNA, microRNA (miRNA) and small interfering RNA (SiRNA), may be used in RNA interference. These small RNAs can direct enzyme complexes to degrade messenger RNA (mRNA) molecules and thus decrease their activity by preventing translation, via post-transcriptional gene silencing. Moreover, transcription can be inhibited via the pre-transcriptional silencing mechanism of RNAi, through which an enzyme complex catalyses DNA methylation at genomic positions complementary to complexed siRNA or miRNA.

RNAi is a technology based on the principle that small, specifically designed, chemically synthesized double-stranded RNA fragments can mediate specific messenger RNA (mRNA) degradation in the cytoplasm and hence selectively inhibit the synthesis of specific proteins. This technology has emerged as a very powerful tool to develop new compounds aimed at blocking and/or reducing anomalous activities in defined proteins. Compounds based on RNA interference can be rationally designed to block expression of any target gene, including genes for which traditional small molecule inhibitors cannot be found.

RNAi has been shown to occur in mammalian cells, not only through long double-stranded RNA (dsRNA) but by means of double-stranded siRNAs. siRNAs are molecules of double-stranded RNA of 21-25 nucleotides that originate from a longer precursor dsRNA.

The mechanism of RNAi is initiated when dsRNAs are processed by an RNase Ill-like protein known as Dicer. Precursor dsRNAs may be of endogenous origin, in which case they are referred to as miRNAs (encoded in the genome of the organism) or of exogenous origin (such as viruses or transgenes). The protein Dicer typically contains an N-terminal RNA helicase domain, an RNA-binding so-called Piwi/Argonaute/Zwille (PAZ) domain, two RNase III domains and a double-stranded RNA binding domain (dsRBD) and its activity leads to the processing of the long double stranded RNAs into 21-24 nucleotide double stranded siRNAs with 2 base 3′ overhangs and a 5′ phosphate and 3′ hydroxyl group. Of the two strands of siRNA, only one, referred to as the guide strand, is incorporated into the enzymatic complex RISC (RNA-induced silencing complex), while the other strand is degraded. The thermodynamic characteristics of the 5′ end of the siRNA determine which of the two strands is incorporated into the RISC complex. The strand that is less stable at the 5′ end is normally incorporated as the guide strand, either because it has a higher content of AU bases or because of imperfect pairings. The guide strand must be complementary to the mRNA to be silenced in order for post-transcriptional silencing to occur.

The resulting siRNA duplexes are then incorporated into the effector complex RISC, where the antisense or guide strand of the siRNA guides RISC to recognize and cleave target mRNA sequences upon adenosine-triphosphate (ATP)-dependent unwinding of the double-stranded siRNA molecule through an RNA helicase activity. The catalytic activity of RISC, which leads to mRNA degradation, is mediated by the endonuclease Argonaute 2 (AG02). AG02 belongs to the highly conserved Argonaute family of proteins. Argonaute proteins are −100 KDa highly basic proteins that contain two common domains, namely PIWI and PAZ domains. The PIWI domain is crucial for the interaction with Dicer and contains the nuclease activity responsible for the cleavage of mRNAs. AG02 uses one strand of the siRNA duplex as a guide to find messenger RNAs containing complementary sequences and cleaves the phosphodiester backbone between bases 10 and 1 1 relative to the guide strand's 5′ end. An important step during the activation of RISC is the cleavage of the sense or passenger strand by AG02, removing this strand from the complex. Crystallography studies analyzing the interaction between the siRNA guide strand and the PIWI domain reveal that it is only nucleotides 2 to 8 that constitute a “seed sequence” that directs target mRNA recognition by RISC, and that a mismatch of a single nucleotide in this sequence may drastically affect silencing capability of the molecule. Once the mRNA has been cleaved, and due to the presence of unprotected RNA ends in the fragments, the mRNA is further cleaved and degraded by intracellular nucleases and will no longer be translated into proteins while RISC will be recycled for subsequent rounds. This constitutes a catalytic process leading to the selective reduction of specific mRNA molecules and the corresponding proteins. It is possible to exploit this native mechanism for gene silencing with the purpose of regulating any gene(s) of choice by directly delivering siRNA effectors into the cells or tissues, where they will activate RISC and produce a potent and specific silencing of the targeted mRNA.

The siRNA can also be referred to as RNAi. The siRNA is a double-stranded RNA of between 21 and 25 nucleotides, but is not limited to this number of nucleotides.

As has been described, the Dicer enzyme cleaves the dsRNA into double-stranded fragments of approximately 21-25 nucleotides (siRNA), with the 5′ end phosphorylated and two unpaired nucleotides protruding at the 3′ end. Of the two strands of siRNA, only one, referred to as the guide strand, is incorporated into the enzymatic complex RISC, while the other is degraded. The thermodynamic characteristics of the 5′ end of the siRNA determine which of the two strands is incorporated into the RISC complex. The strand that is less stable at the 5′ end is normally incorporated as the guide strand. The guide strand must be complementary to the mRNA that is to be silenced in order for post-transcriptional silencing to occur. Subsequently, the RISC complex binds to the complementary mRNA of the guide strand of the siRNA present in the complex, and cleavage of the mRNA occurs.

A skilled person is able to design siRNA based on the GBP1 nucleic acid sequence, for example a sequence described herein. Such RNA molecules may be used according to the various aspects of the invention.

In an embodiment of the invention there is provided a genetically altered legume plant wherein the expression of the GBP1 nucleic acid sequence is reduced or abolished in said plant using RNAi silencing. Also envisaged are methods set out above, e.g. for increasing biomass or generating a plant with a mutant GBP1 nucleic acid sequence using RNA silencing.

### Constructs for Making Plants by Genome Editing

As explained above, in some embodiments, the methods of the invention use gene editing using sequence specific endonucleases that target a GBP1 gene in a plant of interest. As also explained, Cas9 and gRNA may be comprised in a single or two expression vectors. The sgRNA targets the GBP1 nucleic acid sequence.

Thus, in another aspect of the invention, there is provided a nucleic acid construct comprising a nucleic acid sequence encoding at least one DNA-binding domain that can bind to a GBP1 gene. The GBP1 gene comprises and of SEQ ID NOs. 1 to 48 or a functional variant, homolog or orthologue thereof as explained herein.

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one GBP1 nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

In one embodiment, the nucleic acid sequence encodes at least one protospacer element.

In one embodiment, the construct further comprises a nucleic acid sequence encoding a CRISPR RNA (crRNA) sequence, wherein said crRNA sequence comprises the protospacer element sequence and additional nucleotides. In one embodiment, the construct further comprises a nucleic acid sequence encoding a transactivating RNA (tracrRNA).

In a further embodiment, the construct encodes at least one single-guide RNA (sgRNA), wherein said sgRNA comprises the tracrRNA sequence and the crRNA sequence, wherein the sgRNA comprises or consists of a sequence selected from any of SEQ IDs 45 to 60 listed herein, depending on the species targeted. PAM sequences are also shown in the in the section entitled sequences listing. The sgRNA can be used for manipulation of Legume crops. In another aspect of the invention, there is provided a nucleic acid construct comprising a DNA donor nucleic acid wherein said DNA donor nucleic acid is operably linked to a regulatory sequence. The regulatory sequence may be one or more of the following: intron, promoter and/or terminator.

Cas9 and sgRNA may be combined or in separate expression vectors (or nucleic acid constructs, such terms are used interchangeably). Similarly, Cas9, sgRNA and the donor DNA sequence may be combined or in separate expression vectors. In other words, in one embodiment, an isolated plant cell is transfected with a single nucleic acid construct comprising both sgRNA and Cas9 or sgRNA, Cas9 and the donor DNA sequence as described in detail above. In an alternative embodiment, an isolated plant cell is transfected with two or three nucleic acid constructs, a first nucleic acid construct comprising at least one sgRNA as defined above, a second nucleic acid construct comprising Cas9 or a functional variant or homolog thereof and optionally a third nucleic acid construct comprising the donor DNA sequence as defined above. The second and/or third nucleic acid construct may be transfected before, after or concurrently with the first and/or second nucleic acid construct. The advantage of a separate, second construct comprising a Cas protein is that the nucleic acid construct encoding at least one sgRNA can be paired with any type of Cas protein, as described herein, and therefore is not limited to a single Cas function (as would be the case when both Cas and sgRNA are encoded on the same nucleic acid construct).

In one embodiment, a construct as described above is operably linked to a promoter, for example a constitutive promoter.

In another embodiment, the nucleic acid construct further comprises a nucleic acid sequence encoding a CRISPR enzyme. Preferably, the CRISPR enzyme is a Cas protein. More preferably, the Cas protein is Cas9 or a functional variant thereof.

In an alternative embodiment, the nucleic acid construct encodes a TAL effector. Preferably, the nucleic acid construct further comprises a sequence encoding an endonuclease or DNA-cleavage domain thereof. More preferably, the endonuclease is FokI.

In another aspect of the invention there is provided a single guide (sg) RNA molecule wherein said sgRNA comprises a crRNA sequence and a tracrRNA sequence.

In one embodiment, the sgRNA molecule may comprise at least one chemical modification, for example that enhances its stability and/or binding affinity to the target sequence or the crRNA sequence to the tracrRNA sequence. For example, the crRNA may comprise a phosphorothioate backbone modification, such as 2′-fluoro (2′-F), 2′-O-methyl (2′-O-Me) and S-constrained ethyl (CET) substitutions.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by an endoribonuclease cleavage site. The term ‘variant’ refers to a nucleotide sequence where the nucleotides are substantially identical to one of the above sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above described sequences, i.e. SEQ ID NOs. 1-48. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissue-specific promoter. In one embodiment, suitable plant promoters are selected from, but not limited to, cestrum yellow leaf curling virus (CmYLCV) promoter or switchgrass ubiquitin 1 promoter (PvUbil) wheat U6 RNA polymerase III (TaU6) CaMV35S, wheat U6, Arabidopsis or maize ubiquitin (e.g. Ubi 1, 3 or 10) promoters. Alternatively, expression can be specifically directed to particular tissues of seeds through gene expression-regulating sequences.

The nucleic acid construct of the present invention may also further comprise a nucleic acid sequence that encodes a CRISPR enzyme. In a specific embodiment Cas9 is codon-optimised Cas9. In another embodiment, the CRISPR enzyme is a protein from the family of Class 2 candidate proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides or Streptococcus thermophiles.

The term “functional variant” as used herein with reference to Cas9 refers to a variant Cas9 gene sequence or part of the gene sequence which retains the biological function of the full non-variant sequence, for example, acts as a DNA endonuclease, or recognition or/and binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active.

In a further embodiment, the Cas9 protein has been modified to improve activity. Suitable homologs or orthologs can be identified by sequence comparisons and identifications of conserved domains. The function of the homolog or ortholog can be identified as described herein and a skilled person would thus be able to confirm the function when expressed in a plant. In a further embodiment, the Cas9 protein has been modified to improve activity. For example, in one embodiment, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR variant. Alternatively, the Cas protein may comprise a mutation in both nuclease domains, HNH and RuvC-like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains.

In a further embodiment, a Cas protein, such as Cas9 may be further fused with a repression effector, such as a histone-modifying/DNA methylation enzyme or a Cytidine deaminase to effect site-directed mutagenesis. In the latter, the cytidine deaminase enzyme does not induce dsDNA breaks, but mediates the conversion of cytidine to uridine, thereby effecting a C to T (or G to A) substitution. These approaches may be particularly valuable to target glutamine and proline residues in gliadins, to break the toxic epitopes while conserving gliadin functionality.

In a further embodiment, the nucleic acid construct comprises an endoribonuclease. Preferably the endoribonuclease is Csy4 (also known as Cas6f) and more preferably a codon optimised csy4. In one embodiment, where the nucleic acid construct comprises a Cas protein, the nucleic acid construct may comprise sequences for the expression of an endoribonuclease, such as Csy4 expressed as a 5′ terminal P2A fusion (used as a self-cleaving peptide) to a Cas protein, such as Cas9.

In one embodiment, the Cas protein, the endoribonuclease and/or the endoribonuclease-Cas fusion sequence may be operably linked to a suitable plant promoter. Suitable plant promoters are already described above, but in one embodiment, may be the Zea mays Ubiquitin 1, Arabidopsis Ubiquitin1 and Ubiquitin 3 promoters.

Suitable methods for producing the CRISPR nucleic acids and vectors system are known, and for example are published in Molecular Plant (Ma et al., 2015, Molecular Plant, 2015 August; 8(8): 1274-8), which is incorporated herein by reference.

In a further aspect of the invention, there is provided an isolated plant cell transfected with at least one nucleic acid construct as described herein. In one embodiment, the isolated plant cell is transfected with at least one nucleic acid construct as described herein and a second nucleic acid construct, wherein said second nucleic acid construct comprises a nucleic acid sequence encoding a Cas protein, preferably a Cas9 protein or a functional variant thereof. Preferably, the second nucleic acid construct is transfected before, after or concurrently with the first nucleic acid construct described herein.

In an alternative aspect of the invention, the nucleic acid construct comprises at least one nucleic acid sequence that encodes a TAL effector.

In a further aspect of the invention there is provided a genetically modified plant, wherein said plant comprises the transfected cell as described herein. Preferably, the nucleic acid encoding the sgRNA and/or the nucleic acid encoding a Cas protein is integrated in a stable form.

Also included in the scope of the invention, is the use of the nucleic acid constructs (CRISPR constructs) described above or the sgRNA molecules in any of the above described methods. For example, there is provided the use of the above CRISPR constructs or sgRNA molecules to modulate GBP1 activity as described herein. In particular, as described herein, the CRISPR constructs may be used to create dominant loss of function alleles.

In a yet further aspect of the invention there is provided a method of altering root growth in a plant, the method comprising introducing and expressing in a plant a nucleic acid construct as described herein. In another aspect of the invention there is provided a method for obtaining the genetically modified plant as described herein, the method comprising:
a. selecting a part of the plant;
b. transfecting at least one cell of the part of the plant of paragraph (a) with the nucleic acid construct as described above;
c. regenerating at least one plant derived from the transfected cell or cells; selecting one or more plants obtained according to paragraph (c) that show altered root growth.

### Isolated Mutant Nucleic Acids/Protein

The invention also relates to an isolated mutant GBP1 nucleic acid sequence encoding a mutant GBP1 protein wherein expression of the GBP1 nucleic acid sequence or function of the encoded GBP1 protein is reduced or abolished in a plant.

In one embodiment, the isolated mutant GBP1 nucleic acid sequence is mutated compared to a wild type sequence, e.g. SEQ ID NOs. 1 to 48 or a homologue, orthologue or functional variant thereof as defined elsewhere herein. Thus, the GBP1 nucleic acid may be that of a legume plant. Examples of wild type GBP1 nucleic acid sequences are listed elsewhere herein and include SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48. Examples of wild type GBP1 amino acid sequences are listed elsewhere herein and include SEQ ID NOs: 49, 50, 51, 52, 53, 54, 55, 56, 57, 58, 59, 60, 61, 62, 63, 64, 65, 67, 68, 69, 70, 71, 72, 73, 74, 75, 76, 77, 78, 79, 80, 81, 82, 83, 84, 85, 86, 88, 89, 90, 91, 92, 93, 94, 95, 96.

Examples of dominant loss of function mutations are described herein. However, any mutation that results in a dominant loss of function as described herein is encompassed within the scope of the invention. As used herein, “dominant” also encompasses “semi-dominant” or “partially dominant”. Therefore, the mutant allele may be fully dominant, partially dominant or semi-dominant. Preferably, the mutant allele is fully dominant.

The invention also relates to a vector comprising an isolated nucleic acid described above.

The invention also relates to a host cell comprising an isolated nucleic acid or vector as described above. The host cell may be a plant cell or a microbial cell. The host cell may be a bacterial cell, such as Agrobacterium tumefaciens, Agrobacterium rhizogenes or an isolated plant cell. The invention also relates to a culture medium or kit comprising a culture medium and an isolated host cell as described below.

In a related aspect of the invention a functional variant, homolog or orthologue of the nucleic acid sequence encoding GBP1 can be identified by determining the upregulation of expression of the nucleic acid sequence during nitrogen fixing symbiosis.

In a further related aspect of the invention a functional variant, homolog or orthologue of the nucleic acid sequence encoding GBP1 can be identified by measuring the acetylene reduction activity of a plant comprising a loss of function mutation in the functional variant, homolog or orthologue of the GBP1 gene and comparing this activity to the activity of a wild type plant.

Methods and Kits for Identifying a Plant with Altered Root Growth

The invention also relates to a method for identifying a plant, for example a legume plant, with altered nitrogen fixing symbiosis compared to a control plant comprising detecting in a population of plants or plant germplasm one or more polymorphisms in a GBP1 nucleic acid sequence (SEQ ID NOs. 1 to 48) wherein the control plant is homozygous for a GBP1 nucleic acid that encodes a protein having a wild type GBP1 protein (SEQ ID NOs: 49 to 98). In one embodiment, the polymorphism is an insertion, deletion and/or substitution.

In one embodiment, the method further comprises introgressing the chromosomal region comprising at least one polymorphism in the GBP1 gene into a second plant or plant germplasm to produce an introgressed plant or plant germplasm.

A further aspect of the invention provides a detection kit for determining the presence or absence of a polymorphism in a GBP1 nucleic acid sequence in a legume plant, for example a GBP1 nucleic acid as described herein.

The various aspects of the invention described herein clearly extend to any plant cell or any plant produced, obtained or obtainable by any of the methods described herein, and to all plant parts and propagules thereof unless otherwise specified. The present invention extends further to encompass the progeny of a mutant plant cell, tissue, organ or whole plant that has been produced by any of the aforementioned methods, the only requirement being that progeny exhibit the same genotypic and/or phenotypic characteristic(s) as those produced by the parent in the methods according to the invention. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention is further described by the following numbered aspects:
1. A genetically altered legume plant wherein expression of a GBP1 nucleic acid sequence or function of the encoded GBP1 protein is reduced or abolished in said plant.
2. The genetically altered legume plant of aspect 1 wherein said plant comprises a mutation in the GBP1 nucleic acid sequence encoding the GBP1 protein or in a promoter nucleic acid sequence that regulates expression of GBP1.
3. The genetically altered legume plant of aspect 1 or 2 wherein said GBP1 nucleic acid sequence is selected from SEQ ID NOs: 1 to 48 or a homologue, paralogue, orthologue, or functional variant thereof with 70%, 80%, 90% or 95% sequence identity to any one of SEQ ID NOs: 1 to 48.
4. The genetically altered legume plant according to a preceding aspect wherein said mutation comprises the deletion, insertion, replacement or addition of one or more nucleic acids into the nucleic acid sequence.
5. The genetically altered legume plant according to a preceding aspect wherein said mutation comprises the insertion of a Tnt-transposon into the nucleic acid sequence.
6. The genetically altered legume plant of any preceding aspect wherein said plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum).
7. The genetically altered legume plant of any preceding aspect wherein the mutation is introduced using targeted genome modification.
8. The genetically altered legume plant of aspect 7 wherein said mutation is introduced using a rare-cutting endonuclease, for example a TALEN, ZFN or CRISPR/Cas9.
9. The genetically altered legume plant of any preceding aspect wherein the mutation modifies symbiosis with a rhizobacterium in root nodules of the plant.
10. The genetically altered legume plant of any preceding aspect wherein the mutation modifies symbiosis with a rhizobacterium which increases the nitrogen fixing in root nodules of the plant.
11. The genetically altered legume plant of any preceding aspect wherein the plant is heterozygous or homozygous for the mutation.
12. The genetically altered legume plant of any preceding aspect wherein the expression of the GBP1 nucleic acid sequence is reduced or abolished in said plant using RNAi silencing.
13. A method for modulating nitrogen fixing symbiosis in a legume plant and/or increasing plant biomass, the method comprising reducing or abolishing the expression of a GBP1 nucleic acid sequence encoding a GBP1 protein and/or reducing or abolishing the function of the GBP1 protein or a homologue, paralogue, orthologue, or functional variant thereof.
14. The method of aspect 13 wherein the method comprises introducing a mutation in the GBP1 nucleic acid sequence encoding the GBP1 protein or in a promoter nucleic acid sequence that regulates expression of GBP1.
15. The method of aspect 13 or 14 wherein said GBP1 nucleic acid sequence selected from SEQ ID NOS: 1 to 48 or a homologue, paralogue, orthologue, or functional variant with at least 70%, 80%, 90% or 95% sequence identity to any one of SEQ ID NOs: 1 to 48.
16. The method of any of aspects 13 to 15 wherein said mutation comprises the deletion, insertion, replacement and/or addition of one or more nucleic acids into the nucleic acid sequence.
17. The method of any of aspects 13 to 15 wherein said mutation comprises the insertion of a Tnt-transposon into the nucleic acid sequence selected from SEQ ID NOs: 1 to 48.
18. The method of any of aspects 13 to 17 wherein the method comprises introducing said mutation using targeted genome modification.
19. The method of aspect 18 wherein the method comprises introducing said mutation using a rare-cutting endonuclease, for example a TALEN, ZFN or CRISPR/Cas9.
20. The method of any of aspects 13 to 19 wherein the method introduces a heterozygous or homozygous mutation into the plant.
21. The method of aspect 13 wherein the method comprises applying a mutagenic composition to the plant.
22. The method of aspect 13 wherein the method comprises introducing into said plant a dsRNA molecule suitable for RNAi silencing.
23. The method of any of aspects 13 to 22 wherein said plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum).
24. An isolated mutant GBP1 nucleic acid sequence encoding a mutant GBP1 protein wherein expression of the GBP1 nucleic acid sequence or function of the encoded GBP1 protein is reduced or abolished in a plant.
25. The isolated mutant GBP1 nucleic acid sequence of aspect 24 wherein the mutant GBP1 nucleic acid comprises a mutation in the GBP1 nucleic acid sequence selected from SEQ ID NOs: 1 to 48 or a homologue, paralogue, orthologue, or functional variant with at least 70%, 80%, 90% or 95% sequence identity thereto.
26. The isolated mutant of GBP1 nucleic acid sequence of aspect 23 wherein the mutant GBP1 nucleic acid sequence comprises a deletion, insertion, addition and/or replacement of one or more nucleic acids and/or a Tnt-transposon inserted into the nucleic acid sequence selected from SEQ ID NOs: 1 to 48.
27. The isolated mutant of GBP1 nucleic acid sequence of any aspects 22 to 26 wherein the mutant GBP1 nucleic acid sequence is from a plant selected from Medicago, Pea (Pisum sativum, 2), Broad bean (Vicia faba, 1), Clover (Trifolium pratense, 1), Birds treefoil (Lotus japonicus, 1), Lupinus angustifolius, Cowpea (Vigna unguiculata, 3), Common Bean (Phaseolus vulgaris, 3), Soybean (Glycine max, 6), Cajanus cajan, and Chickpea (Cicer arinetum, 1).
28. A vector comprising an isolated nucleic acid of any of aspects 23 to 27.
29. A host cell comprising a vector of aspect 28.
30. A method for producing a plant with modulated nitrogen fixing symbiosis, comprising introducing a mutation into a GBP1 nucleic acid or in a promoter nucleic acid sequence that regulates expression of GBP1.
31. The method of aspect 28, comprising introducing a mutation in the GBP1 nucleic acid sequence selected from SEQ ID NOS: 1 to 48 or a homologue, paralogue, orthologue, or functional variant with at least 70%, 80%, 90% or 95% sequence identity thereto.
32. The method of aspect 29, comprising the wherein said mutation comprises the deletion, insertion, replacement and/or addition of one or more nucleic acids into the nucleic acid sequence and/or insertion of a Tnt-transposon into the nucleic acid sequence selected from SEQ ID NOs: 1 to 48.
33. The method of any of aspects 30 to 32, comprising introducing the mutation using targeted genome modification.
34. The method of aspect 33, comprising introducing the mutation using a rare-cutting endonuclease, for example a TALEN, ZFN or CRISPR/Cas9.
35. The method of any of aspects 28 to 34, wherein the method is carried out in a plant selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum).
36. A method for identifying a plant with altered nitrogen fixing symbiosis compared to a control plant, the method comprising detecting in a population of plants one or more polymorphisms in a GBP1 nucleic acid sequence.
37. The method of aspect 36 wherein the GBP1 nucleic acid sequence is selected from SEQ ID NOs: 1 to 48 or a homologue, paralogue, orthologue, or functional variant with about at least 70%, 80%, 90% or 95% sequence identity thereto wherein the control plant comprises a GBP1 nucleic acid that encodes a protein having a wild type GBP1 protein.
38. A detection kit for determining the presence or absence of a polymorphism in aGBP1 nucleic acid sequence in a legume plant.

The invention is further described in the following non-limiting examples.

### EXAMPLES

### Example 1: GBP1 Expression is not Upregulated Following Oomycete or Fungal Infection, or after Laminarin Application

GBP is related to glycosyl hydrolase family 81 genes encoding endo-beta (1,3) glucanases dual domain proteins with glucan-binding and hydrolytic activities towards β-1,3/1,6-glucans (Umemoto et al., 1997; Fliegmann et al., 2004) This family is represented by 12 genes in the model legume Medicago truncatula.

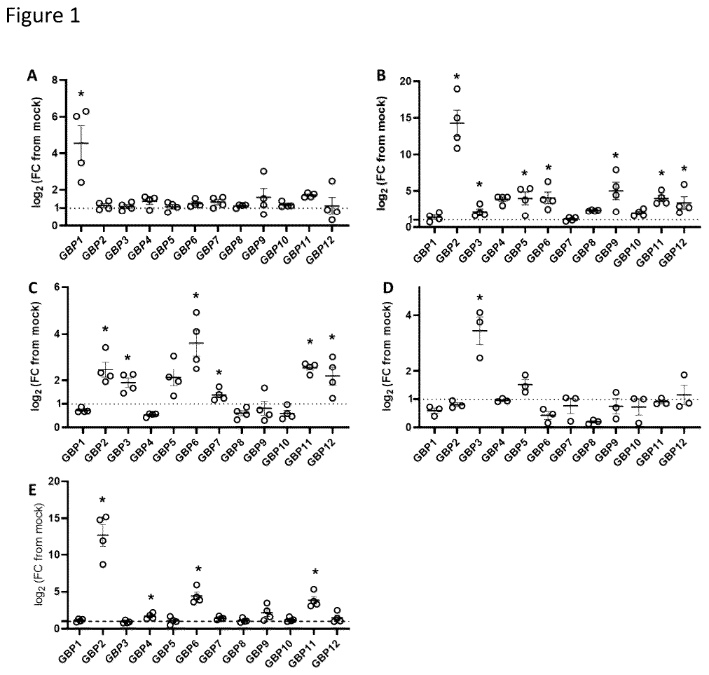

Medicago seedlings were exposed and infected with Sinorhizobium meliloti (FIG. 1, panel A), Rhizoctonia solani (FIG. 1, panel B), the oomycete Phytophthora palmivora (FIG. 1, panel D) or the fungus Botrytis cinerea (FIG. 1, panel C). Panel E of FIG. 1 shows the results of laminarin treatment of GBP gene expression.

### Methods

Bleach sterilised seeds of Medicago truncatula were germinated and transferred on sterile plates with 0.8% agarose. For P. palmivora infection assay two days old seedlings were inoculated with, 10 μl of P. palmivora zoospore suspension (5×10⁴ zoospores/ml). 24 hours after inoculation, infected roots were pooled into four biological samples for RNA extraction. For B. cinerea infection assay five days old seedlings were inoculated with, 100 μl of B. cinerea spore suspension (5×10⁴ spores/ml). 48 hours after inoculation, infected roots were pooled into four biological samples for RNA extraction using the RNeasy Mini Kit including on-column DNAse digest according to manufacturer recommendations (Qiagen). Reverse transcription and cDNA synthesis were performed on 1 μg of total RNA using the iScript cDNA Kit according to manufacturer recommendations (Bio-Rad). Quantitative PCR (qPCR) was performed in technical triplicates using SYBR Green I Master kit in a LightCycler® 480 (Roche). Ten microliter reaction volumes were used with 7.5 μl of master mix containing 1 μM gene specific primers and 2.5 μl of 10-fold pre-diluted cDNA.

### Results

As shown in FIG. 1 several of the GBP genes are upregulated in response to plant or root exposure to fungal (B. cinerea) and oomycete (P. palmivora) pathogens. Expression of GBP3 is induced in response to exposure to the oomycete P. palmivora. Expression of GBP2, GBP3, GBP5, GBP6, GBP7, GBP11 and GBP12 is induced in response to exposure to the fungus B. cinerea. Noticeably, expression of GBP1 was not found to be induced in response to fungal or oomycete exposure.

Medicago seedlings were also exposed to laminarin in order to determine whether expression of any members of the GBP family was induced in response (FIG. 1, panel E).

### Method

Bleach sterilised seeds of Medicago truncatula were germinated and transferred on sterile plates with 0.8% agarose. Four days after germination each seedling was treated with 100 μl of 4 μM solution of laminarin (MERCK, L9634). After two hours of treatment, roots were pooled into four biological samples. RNA extraction, cDNA synthesis and qPCR were performed as described before.

### Results

As shown in panel E of FIG. 1 the expression of GBP2, GBP6, GBP11 and to a lesser extent GBP9 was induced in response to exposure to laminarin. Expression of GBP1 was not induced in response to laminarin exposure.

The results of these experiments show that although expression of the majority of the GBP family of genes is induced as a result of pathogenic or laminarin exposure, expression of GBP1 is not induced. This indicates that GBP1 is not involved in an immune response to pathogenic infection.

### Example 2: GBP1 is Strongly Upregulated in Nodules During Nitrogen Fixing Symbiosis

Medicago seedings were grown in the presence of the nitrogen fixing symbiotic rhizobacteria S. meliloti and the expression of the GBP family of genes was measured (FIG. 1, panel A).

### Method

Germinated seeds of Medicago truncatula were sown on 1:1:1 mix of vermiculite, Terragreen and perlite saturated with Farhaeus medium (1 mM MgSO4·7H2O, 0.75 mM KH₂PO₄, 1 mM Na₂HPO₄, UM Fe-citrate, 0.75 mM Ca(NO₃)₂, 0.7 mM CaCl₂), 0.35 μM CuSO₄·5H₂O, 4.69 μM MnSO₄·7H₂O, 8.46 μM ZnSO₄·7H₂O, 51.3 μM H₃BO₃, 4.11 μM Na₂MoO₄·2H₂O, pH 6.7) and grown in a growth chamber at 21° C. and 16/8-h light/darkness. Three days after germination plants were inoculated with Sinorhizobium meliloti 2011 (OD600 0.1, 2 mL per plant). Nodulated roots were collected for analysis 21 days after inoculation. RNA extraction, cDNA synthesis and qPCR were performed as described before.

### Results

As shown in panel A of FIG. 1, GBP1 is strongly upregulated in root nodules during the nitrogen fixing symbiosis indicating that the role of GBP1 is distinct from other members of the GBP family. In order to confirm that the expression of GBP1 was induced during nitrogen fixing symbiosis Medicago roots expressing GBP1 GFP fluorescent promoter-reporter construct were generated. These seedlings were cultivated in the presence of S. meliloti as described above. The only difference being that the S. meliloti was tagged with a different fluorescent marker.

### Method

The promoter region of GBP1 gene (2 kb upstream of the translation start) was fused to Green Fluorescent Protein (GFP) with nuclear localization sequence (NLS) and introduced into Medicago roots by Agrobacterium rhizogenes-mediated transformation. Transgenic roots were nodulated by S. meliloti rhizobia expressing Red Fluorescent Protein (RFP). For imaging, colonized roots and root nodule sections were mounted in water and covered by coverslips. Imaging was done by using a Leica TCS SP8 confocal microscope with emission/excitation settings 510/488 nm for GFP and 585/608 nm for RFP.

### Results

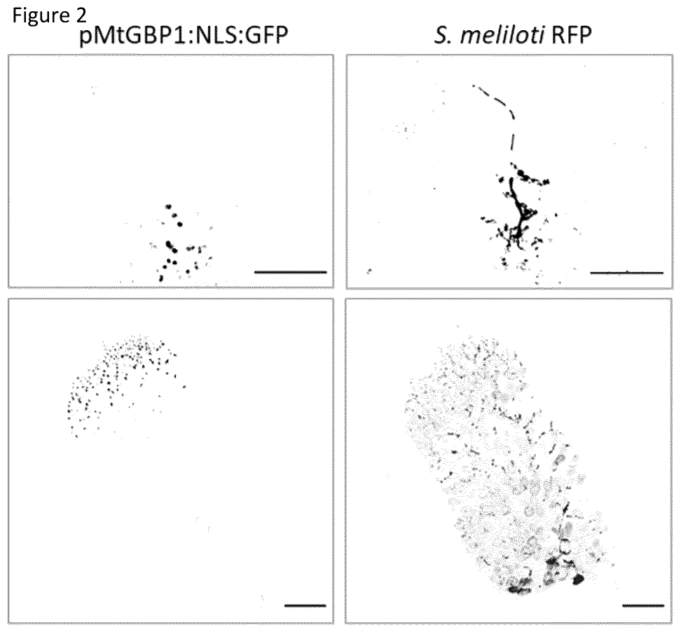

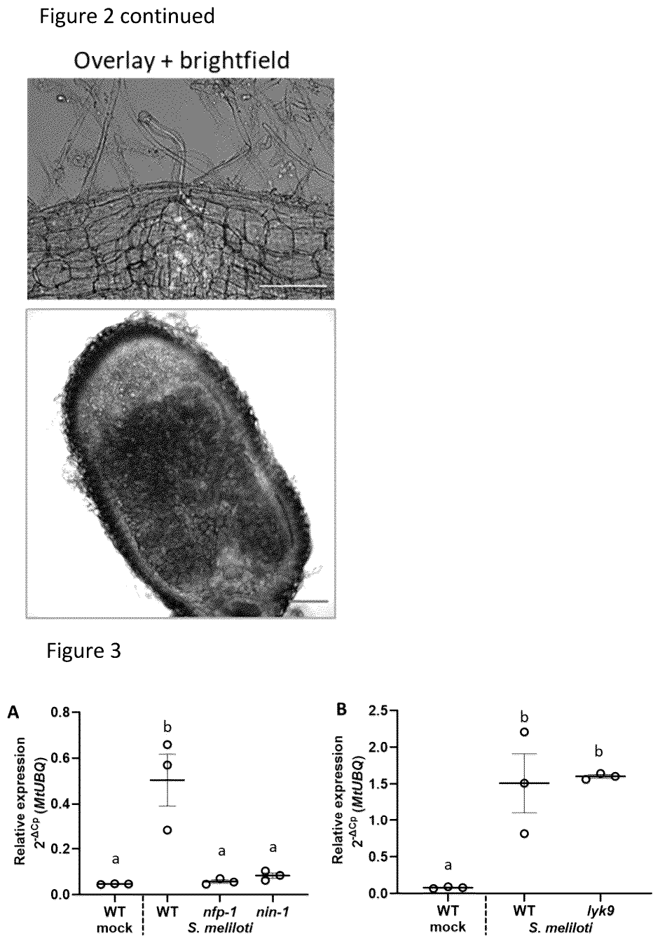

The promoter-reporter constructs show that the GBP1 gene is active during the early stages of rhizobacterial entry into the root (FIG. 2, left image). Expression of GBP1 occurs in the root with entry of rhizobacteria into the root via the infection thread passing through the root hair and into the nodule primordium. In fully developed nodules (FIG. 2, right image) GBP1 expression is limited to the zones where bacteria release into plant cells and develop into bacteroides. Bacteroides are the nitrogen fixing organelle-like intracellular structure that contain the majority of the symbiotic nitrogen fixing bacteria present in the legume root system.

### Example 3: GBP1 Induction Relies on the Common Symbiosis Signalling Pathway

Symbiosis and defence-associated receptor Medicago mutants (NIN and NFP loss of function mutants) were investigated against wild type Medicago to determine whether GBP1 was related to the Common Symbiosis Signalling Pathway. Medicago mutant and wildtype seedlings were cultivated in the presence of the nitrogen fixing symbiotic rhizobacteria S. meliloti and the expression of GBP1 was measured (FIG. 3).

### Method

Germinated seeds of Medicago mutants nfp-1, nin-1, lyk9 were sawn on 1:1:1 mix of vermiculite, Terragreen and perlite saturated with Farhaeus medium and grown in a growth chamber at 21° C. and 16/8-h light/darkness. Three days after germination plants were inoculated with Sinorhizobium meliloti 2011 (OD600 0.1, 2 mL per plant). Nodulated roots were collected for analysis 4 days after inoculation. RNA extraction, cDNA synthesis and qPCR were performed as described before.

### Results

As shown in FIG. 3 the Medicago mutants with non-functional NIN or Nod-Factor signalling did not show any induction of GBP1 when cultivated with S. meliloti. This indicates that the induction of GBP1 expression in response to root infection by S. meliloti is requires NIN and Nod-Factor signalling. NIN is a central transcriptional regulator of nitrogen fixing symbiosis (Jiang et al, 2021) and NFP is a key surface receptor which perceived the bacterial Nod-factor to initiate symbiosis in Medicago. Therefore, the finding that expression of GBP1 is not induced in Medicago with non-functional NIN or Nod-Factor signalling in response to cultivation with a symbiotic bacterium indicates that such induction of GBP1 expression is reliant on the activation of Medicago symbiosis signalling.

When Medicago mutants containing an Lyk9 loss of function mutation were cultivated with S. meliloti GBP1 expression was still induced (FIG. 3) indicating that induction of GBP1 expression is Lyk9/CERK1 independent. Lyk9 is a surface chitin receptor associated with the plant defence mechanism (Bozsoki et al, 2017). A loss of function in this receptor did not affect the induction of GBP1 expression in response to cultivation with S. meliloti. This indicates that GBP1 does not appear to have a role in the Lyk9/CERK1 defence against bacterium.

### Example 4: Gene Activation does not Affect Nodule Development

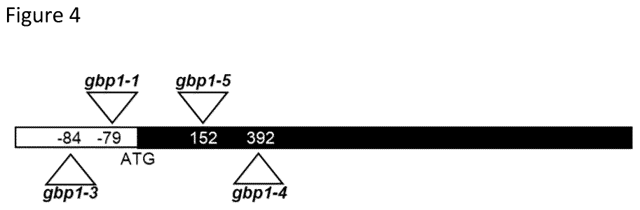

Several mutant Medicago lines were obtained with each line having either an up-regulation of GBP1, a knockout of the GBP1 gene or a transcript that produces non-functional GBP1 protein. A schematic representation of the GBP1 gene in the different Medicago lines is shown in FIG. 4. The lines gbp1-1 and gbp1-3 display an upregulated level of GBP1 transcript. The gbp1-4 line is a GBP1 knockout line and gbp1-5 has a disrupted open reading frame resulting in a truncated, non-functional GBP1 protein.

The ability of these mutant Medicago lines to form nodules was assessed to determine whether GBP1 has any effect on nodule formation.

### Method

Germinated seeds of Medicago mutants gbp1-1, gbp1-3, gbp1-4, gbp1-5 and corresponding wild type lines were sawn on 1:1:1 mix of vermiculite, Terragreen and perlite saturated with Farhaeus medium and grown in a growth chamber at 21° C. and 16/8-h light/darkness. Three days after germination plants were inoculated with Sinorhizobium meliloti 2011 (OD600 0.1, 2 mL per plant).

Nodulated roots were collected for analysis 21 days after inoculation. Nodulation phenotyping and quantification were performed using a Fluorescent Stereo Microscope Leica M165 FC equipped with a DFC310FX camera.

### Results

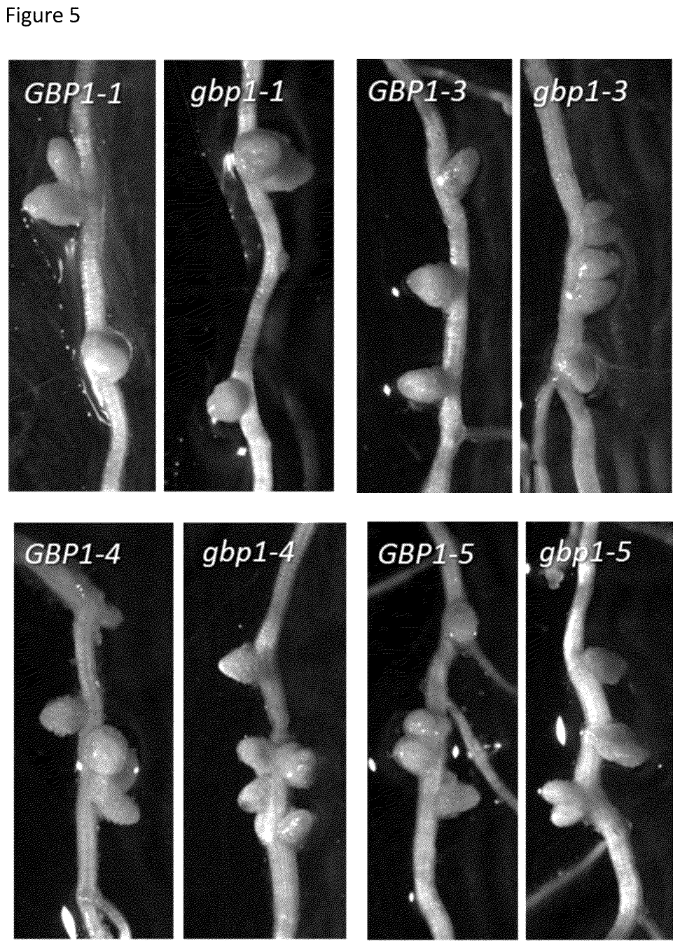

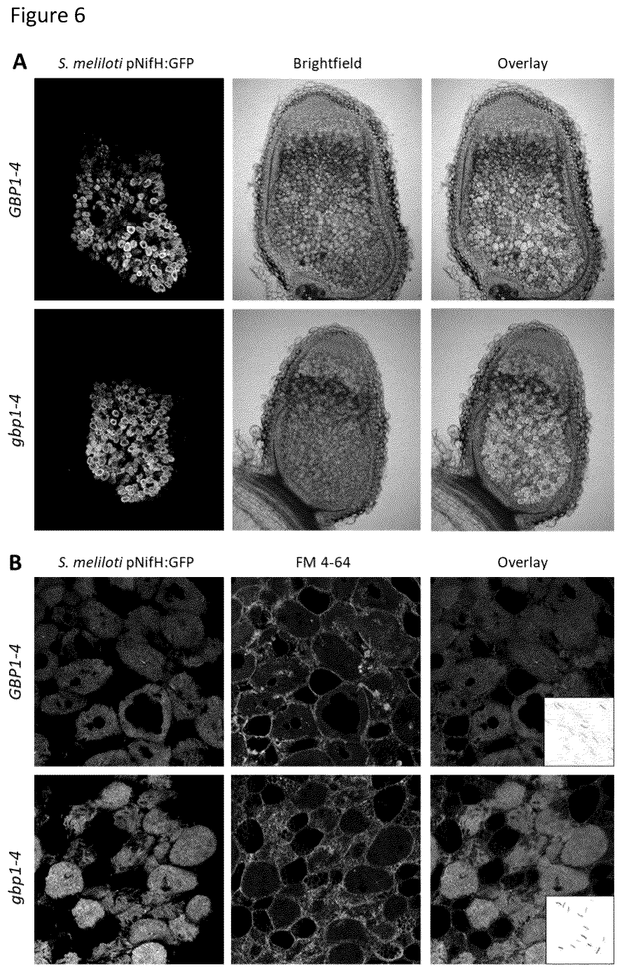

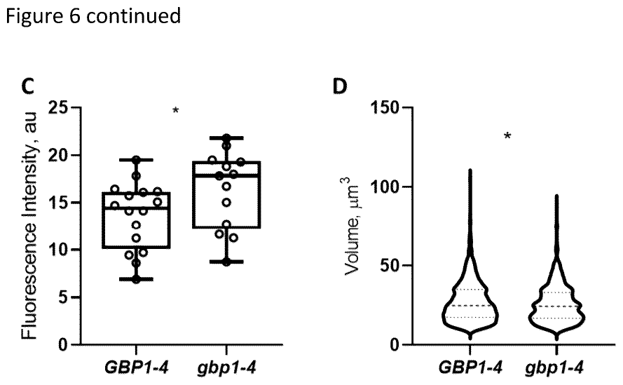

As shown in FIGS. 5 and 6, up-regulation of GBP1 in mutant lines gbp1-1 and gbp1-3 does not affect nodule formation. Also shown in FIGS. 5 and 6 is that knockout or non-functional GBP1 mutant, gbp1-4 and gbp1-5 do not affect nodule formation.

The transposon insertion Medicago line gbp1-4 interrupts the open reading frame of GBP1 inactivating the gene. Medicago plants of the gbp1-4 line do not induce GBP1 upon colonisation with Rhizobacteria. Panels C and D of FIG. 6 show that there is an increase in NifH expression in the gbp1-4 Medicago line compared to wildtype (FIG. 6, panel C) but no increase in the overall volume of each root nodule (FIG. 6, panel D).

### Example 5: Modulation of GBP1 Gene Expression Modulates Nitrogen Fixation and the Amount of Symbiotic Shoot Biomass Increases

A selection of the mutant Medicago lines previously generated were further investigated to determine the effect of either knockout of GBP1 (gbp1-4) or upregulation of GBP1 expression (gbp1-1) has on induction of GBP1 gene expression, nitrogen fixation and root nodule development.

### Induction of GBP1 and Root Nodule Formation in GBP1 Mutant Medicago Lines

### Method

The knockout mutant gbp1-4 was identified in a Tnt1-insertion mutant population of Medicago truncatula ecotype R108. Plants of the Tnt1 insertion line NF1807 were screened for Insertion-17 in the GBP1 gene using PCR with gene specific (GPB1gF3 TAAGGAGAATAAGTAAGTAGCCCTTATCA (SEQ ID NO: 137); GBP1gR2 AGAAGGAGCCCACCAAAGTT (SEQ ID NO: 138)) and Tnt1 retrotransposon specific (tnt1-R CAGTGAACGAGCAGAACCTG (SEQ ID NO: 139); tnt1-F ACAGTGCTACCTCCTCTGGA (SEQ ID NO: 140)) primers.

Homozygous gbp1-4 plants were isolated from a self-pollinated heterozygous gbp1-4/GBP1-4 individual. After, gbp1-4 was backcrossed to R108 wild type and resegregated. Homozygous GBP1-4 progeny of the same parent were isolated and used in subsequent experiments as a wild type control. The effect of the Tnt1 insertion on GBP1 expression was determined by RT-qPCR using gene specific primers (GBP1qF AAATCAATATGTTTGGGTCATGC (SEQ ID NO: 141); GBP1qR TTGTCGGCCACATATCCTTG (SEQ ID NO: 142)).

GBP1-4 and gbp1-4 plants were grown in 1:1:1 mix of vermiculite, Terragreen and perlite saturated with Farhaeus medium (CaCl₂), 0.1 g/l.; MgSO₄×7H₂O, 0.12 g/l.; KH₂PO₄, 0.1 g/l.; Na₂HPO₄×12H₂O, 0.358 g/l; Fe-EDTA 5 ml/l; Mn, Cu, Zn, B, Mo traces; pH 6.7) in a growth chamber at 21° C. and 16/8-h light/darkness. Three days after germination plants were inoculated with Sinorhizobium meliloti 2011 (OD600 0.1, 2 mL per plant). Nodulated roots were collected for analysis 21 days after inoculation.

Nodulation, phenotyping, RNA extraction, cDNA synthesis and qPCR were performed as described above.

GBP1 open reading frame was amplified via PCR from nodule cDNA using Phusion high-fidelity polymerase (Finnzymes) and specific primers GBP1cIF ATGTCTTCATCATCTTCTCTTCCTTT (SEQ ID NO: 143), GBP1cIR TCATCTGCTATGGATCCACC (SEQ ID NO: 144). Amplicons were introduced into pENTR (D-TOPO Cloning Kit, Thermo Fisher Scientific) and used as an entry vector. To generate pUbq: GBP1 construct entry vector was recombined with pENTR:prAtUBQ3 into pKGW-MGW destination vector using LR Clonase Plus (Thermo Fisher Scientific). pUbq: GBP1 was introduced into Medicago roots by Agrobacterium rhizogenes-mediated transformation. Transgenic roots were nodulated by S. meliloti rhizobia expressing GFP. Nodulation phenotyping and quantification were perform using a Fluorescent Stereo Microscope Leica M165 FC equipped with a DFC310FX camera.

### Results

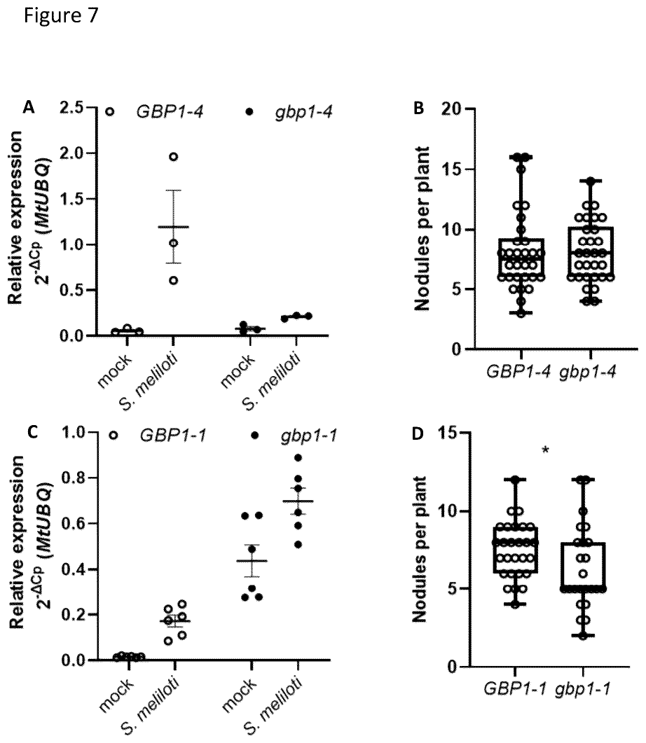

As shown in FIG. 7, knocking out the GBP1 gene (gbp1-4 line) in Medicago so that GBP1 gene expression is not induced when Medicago is cultivated with S. meliloti causes a small increase in the number of root nodules observed per plant compared to the negative control. The gbp1-1 line is a mutant Medicago line in which GBP1 Gene expression is constitutively upregulated but can also be induced in response to cultivation with S. meliloti as show in FIG. 7. The gbp1-1 Medicago line forms fewer root nodules per plant when cultivated with S. meliloti compared to the negative control GBP1-1. The gbp1-1 Medicago line also forms fewer nodules per plant compared to the gbp1-4 Medicago line as shown in FIG. 7.

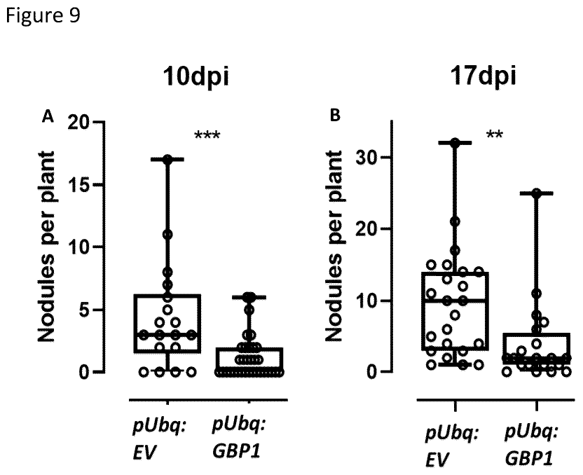

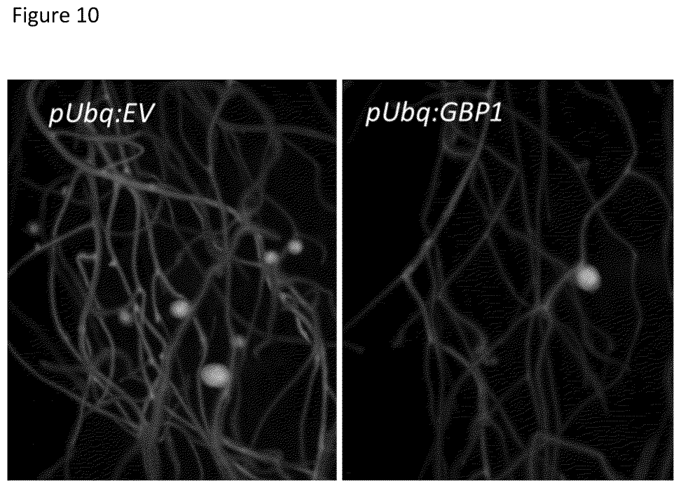

Root systems with constitutive ectopic expression of GBP1 under control of the Ubiquitin promoter (pUbq: GBP1) were independently generated. The number of nodules per Medicago plant in the pUbq: GBP1 plants was compared to a negative control (pUbq: EV). FIG. 9 shows the reduction in number of root nodules per Medicago plant when GBP1 is ectopically constitutively expressed. FIG. 10 is a photograph that shows the reduction in root nodule number observed when GBP1 is ectopically constitutively expressed in Medicago plants. FIGS. 9 and 10 show that the pUbq: GBP1 Medicago plant root systems display strongly reduced root nodule numbers further indicating a role for GBP1 as a negative regulator of nitrogen fixing symbiosis.

### Nitrogen Fixation and Shoot Biomass in GBP1 Mutant Medicago Lines

The acetylene reduction assay is used as a measure of the nitrogen fixing enzymatic activity of the bacteroid nitrogenase per mg root nodule over time. The acetylene reduction assay is a simple and robust assay that relies on the ability of bacterial nitrogenase to reduce acetylene to ethylene which is then directly quantified. Three moles of ethylene produced during the acetylene reduction assay is understood to correspond to one mole of ammonia.

### Method

Nitrogenase activity was measured by the acetylene reduction assay. Nodulated roots were collected into 13 ml tubes. Tubes were stoppered with rubber septa (Suba-Seal n°29) and injected with 1 ml of acetylene into each. After 1 hour of incubation formed ethylene was quantified using a Perkin Elmer Clarus 480 gas chromatograph equipped with a HayeSep N (80-100 MESH) column. The injector and oven temperatures were kept at 100° C., while the FID detector was set at 150° C. The carrier gas (nitrogen) flow was set at 8-10 mL/min. Nitrogenase activity is reported as nmol of ethylene/mg nodules/hour.

### Results

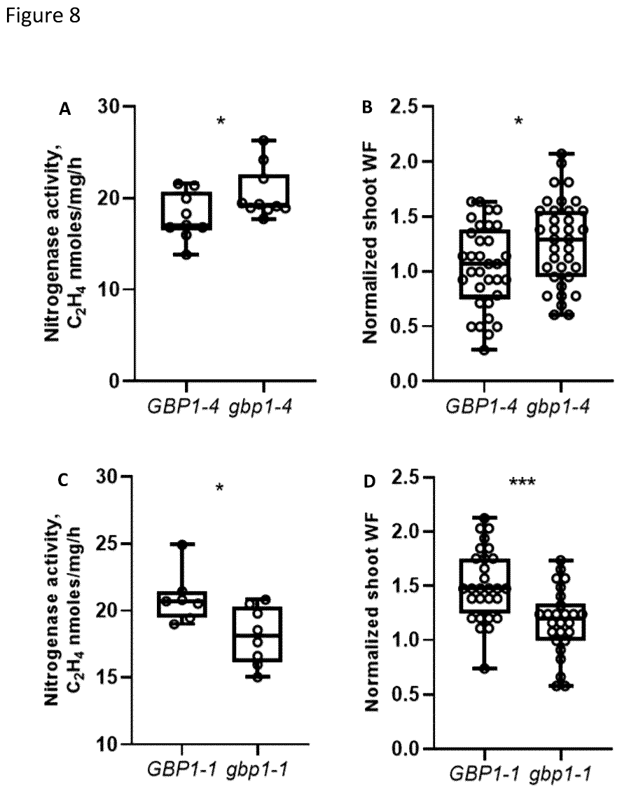

As shown in FIG. 8 the gbp1-4 mutant Medicago line demonstrates an increase in acetylene reduction compared to a negative control (GBP1-4) indicating an increase in nitrogen fixing in the gbp1-4 Medicago mutant line. In contrast to the results obtained for the gbp1-4 line, the gbp1-1 line demonstrated reduced acetylene production compared to the negative control (GBP1-1).

FIG. 8 also shows the biomass of each mutant Medicago line. Plants from the gbp1-4 mutant Medicago line demonstrate an increase in biomass compared to a negative control (GBP1-4). The opposite is seen when the gbp1-1 mutant Medicago line is compared to a negative control (GBP1-1).

The results of the acetylene reduction assay suggest that gbp1-1 root nodules fix less nitrogen per nodule and time than the wildtype Medicago plants. Importantly, nodule number remains unaffected, or increased, in gbp1-4 plants as shown in FIG. 7. This is remarkable because many genes impacting on symbiosis also alter or increase the number of nitrogen fixing nodules in the root system.

The inverse relationship between GBP1 expression and nodule formation (FIG. 9) is mirrored in both the relationship between GBP1 and acetylene reduction and is also mirrored in the relationship between GBP1 and plant biomass. Taking these results together indicates that reducing GBP1 expression in a legume leads to an increase in root nodule formation, an increase in bacterial symbiosis, bacterial nitrogen fixation and in turn plant biomass.

### Example 6: Closely Related GBP1 Orthologs in Other Legumes

Although Medicago is not a high value crop in and of itself, it is an accurate model organism of other high value species. The GBP1 gene of Medicago is highly conserved and orthologs are present in several other legume species which are of high value for human consumption or other industrial uses. Species that have orthologs of GBP1 include but are not limited to Pea (Pisum sativum, 2), Broad bean (Vicia faba, 1), Clover (Trifolium pratense, 1) and Chickpea (Cicer arinetum, 1). Several legumes also display close homologs of GBP1. Species that have a close homolog of GBP1 include but are not limited to Common Bean (Phaseolus vulgaris, 3), Cowpea (Vigna unguiculata, 3), Cajanus cajan, Soybean (Glycine max, 6) and Birds treefoil (Lotus japonicus, 1).

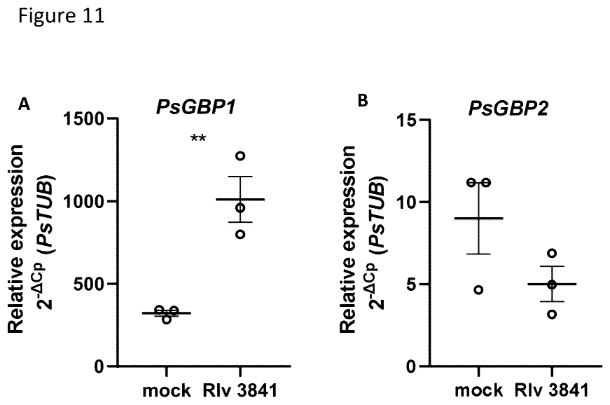

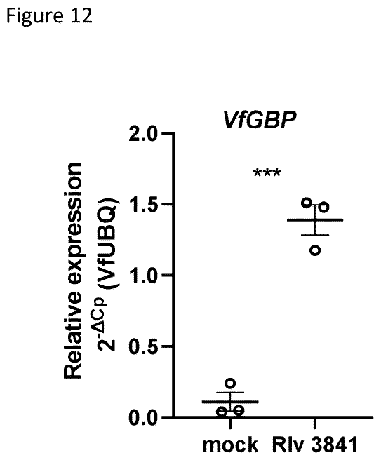

The induction of GBP1 gene expression in Pea (Pisum sativum) was investigated alongside another member of the GBP gene family (GBP2) during cultivation with Rhizobium leguminosarum (RIv3841) as shown in FIG. 11. Induction of GBP in Broad Bean (Vicia faba) was also investigated during cultivation with Rlv3841 as shown in FIG. 12.

### Method

Bleach sterilised and germinated seeds of Pea and broad bean were sown on autoclaved 1:1:1 mix of vermiculite, Terragreen and perlite saturated with Farhaeus medium and grown in a growth chamber at 21° C. and 16/8-h light/darkness. Three days after germination plants were inoculated with Rhizobium leguminosarum bv. viciae 3841 (OD600 0.1, 4 mL per plant). Nodulated roots were collected for analysis 21 days after inoculation. RNA extraction, cDNA synthesis and qPCR were performed as described before

### Results

FIG. 11 shows that the GBP1 expression in Pea was induced in root nodules during symbiosis with the symbiotic bacterium Rlv3841. Induction of GBP2 gene expression was not seen during symbiosis when Pea was cultivated with Rlv3841. The results for Broad Bean indicate that GBP gene expression was also induced during cultivation with Rlv3841.

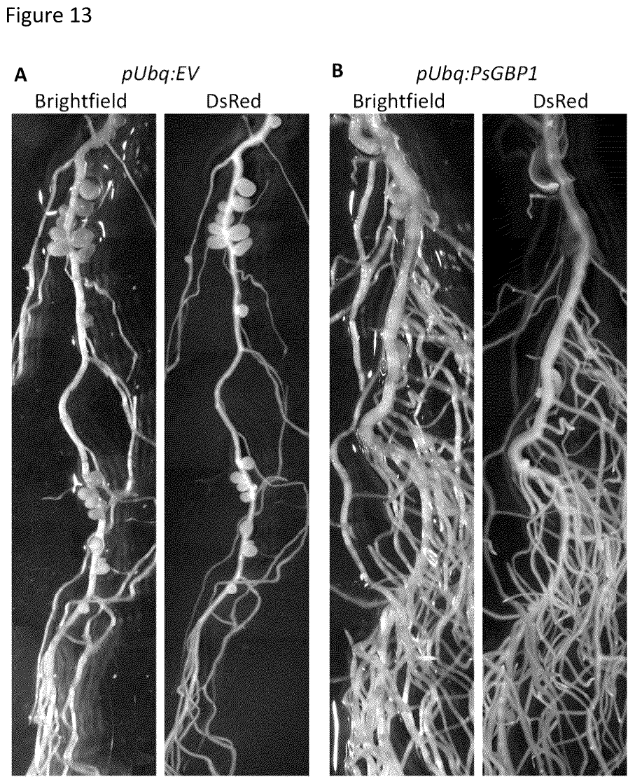

Pea (Pisum sativum) root systems with constitutive ectopic expression of the pea PsGBP1 (Psat3g201680.1) gene under control of the Ubiquitin promoter (pUbq: PsGBP1) were generated, using Agrobacterium rhizogenes-mediated transformation. FIG. 13 shows that the constitutive expression of pea PsGBP1 dramatically reduces root nodulation, further confirming the role of the GBP1 gene as a negative regulator of nitrogen-fixing symbiosis in pea.

Taking the results of the examples above into consideration it is possible to improve legume nitrogen fixation and shoot biomass through the inactivation of GBP1 genes or by attenuating GBP1 expression in legume plants. The correct gene to be inactivated has been identified by the fact that the gene expression of such a gene is upregulated in the root specifically during nitrogen fixing symbiosis. A transcriptional upregulation in GBP1 gene expression has been demonstrated in the root nodules of the model species Medicago alongside both Pea and Broad Bean during nitrogen fixing symbiosis.

### Discussion

Biological nitrogen fixation is the primary source of plant-available nitrogen in most ecosystems [1]. The Rhizobium-legume symbiosis is one of the most productive nitrogen-fixing systems. In this so-called root nodule symbiosis, bacteria live in the root cells of the host plants, where they bind elementary nitrogen from the air in special organs, the nodules. As a result of this symbiosis, legume crops are able to provide themselves and subsequent crops with nitrogen, reducing requirements for mineral nitrogen fertilization, one of the main agricultural practices with very high economic and environmental costs [2].

Despite its agricultural importance, our understanding of symbiosis is largely limited to the signalling necessary for its development and relatively little is known about the mechanisms controlling symbiotic efficiency. On the other hand, the available data clearly point to the important role of plant immunity in the Rhizobium-legume symbiosis. Using plant pathogen-host research as an example, one would expect that knowledge would emerge that could enhance the use of root nodule symbiosis in agriculture. Hypothetically, specific molecular mechanisms negatively regulating nitrogen-fixing symbiosis could evolutionarily derive from plant immunity. Genomic and transcriptomic resources are widely available nowadays, enabling us to address this hypothesis.

Medicago truncatula is a model legume, well-established for Rhizobium-legume symbiosis related studies. Combining a phylogenetic approach with extensive transcriptomic data on Medicago-rhizobia symbiotic interactions we identified a gene encoding β-Glucan-Binding Protein 1 (MtGBP1). GBPs are endo-β-1,3-glucanases, dual-domain proteins with glucan-binding and hydrolytic activities towards microbial β-1,3/1,6-glucans. Previous studies suggest an involvement of GBP proteins in plant immunity and probably recognition of microbial glucans [3,4]. In line with it, our transcriptomic studies have shown activation of GBP genes of Medicago upon root exposure to laminarin (a branched glucan, structurally similar to glucans from cell walls of filamentous pathogens) or upon infection with detrimental fungi like Botrytis cinereal, or the pathogenic oomycete Phytophthora palmivora (MtGBP2, MtGBP3, MtGBP6, MtGBP11, MtGBP12). Surprisingly, MtGBP1 is specifically induced during rhizobia infection and nodule organogenesis suggesting that its transcriptional regulation differs from that of other GBP gene family members. MtGBP1 gene knockout via transposon insertion does not disturb nodule development and morphology. However, the knockout mutant line gbp1-4 with a transposon insertion in the GBP1 open reading frame shows an elevated level of nitrogenase activity measured via acetylene reduction assay and a greater plant biomass under nitrogen-limiting conditions compare to wild type. Vice versa, the Medicago overexpression line gbp1-1 with an expression-activating transposon insertion in the MtGBP1-upstream regulatory region produces less biomass thereby demonstrating the negative role of MtGBP1 in symbiosis.

Our transcriptomic studies show that upregulation of MtGBP1 transcript levels during symbiosis development is dependent on Nod factor signalling and NIN transcriptional regulation, crucial regulatory mechanisms of symbiosis development. On the other hand, the extent of transcriptional upregulation of MtGBP1 during symbiosis also depends on the rhizobia strain and particularly its symbiotic efficiency. A comparative study with three different strains of rhizobia with high, low and almost no nitrogen-fixing ability have shown that the most efficient strain causes the highest increase in MtGBP1 transcripts (5 fold); whereas non-efficient rhizobia cause only slight transcriptional activation (1.5 fold).

Phylogenetic analysis shows that GBP genes are widespread among land plants. However, this gene family is particularly abundant in legumes. Most of the analysed diploid dicot and monocot plants have one, two or three GBP genes, whereas in diploid legumes their amount ranges from six (Lotus japonicus, Cajanus cajan, Lupinus angustifolius) to twelve in Medicago. Gene synteny (the physical localization of genetic loci on the chromosome) of Medicago GBPs suggests that this gene family evolved by mechanisms of tandem duplication. One of the most recent duplications is MtGBP1/MtGBP2. Strikingly, these two proteins share 91.4% of protein similarity but have very different expression patterns suggesting a divergent functionality. It is tempting to speculate that these two genes evolved from an ancestral defence gene through gene duplication and subsequent neo-functionalisation, whereby the MtGBP2 version maintained the ancestral function, and MtGBP1 specialized into a symbiosis regulator.

Since GBP genes occur widely across legumes we looked for evidence of similar mechanisms in economically relevant legumes. Close homologs of MtGBP1 are found in common bean (Phaseolus vulgaris), cowpea (Vigna unguiculata), pigeon pea (Cajanus cajan), soybean (Glycine max) and blue lupin (Lupinus angustifolius). Pea (Pisum sativum) and faba (Vicia faba) bean are the closest relatives of Medicago. Both have similar GBP genes in the same phylogenetic subclade and hence might have the same functionality. Like Medicago MtGBP1, these genes show transcriptional upregulation during colonization by Rhizobium leguminosarum biovar viciae, the specific symbiont of the Fabeae tribe. These findings provide initial evidence that similarly transcriptionally regulated GBP genes can quantitatively modulate nitrogen fixation in grain legumes.

Overall, our work clearly showed that MtGBP1 is a negative regulator of nitrogen fixation, which potentially evolved from a defence related gene to limit the extent of nitrogen fixation of excessively productive microsymbionts. To date, this is a unique example when knockout of the symbiotically induced gene increases nitrogenase activity, resulting in higher biomass production. This finding potentially enables the improvement of nitrogen fixation in legume crops and non-legume crops by gene editing.

### Example 7: Related GBP1 Orthologs in Non-Legumes

Orthologs of GBP1 exist in non-legume plants. Thus, a GBP1 nucleic acid, protein or promoter sequence in a non-legume plant can be manipulated using the techniques herein. This may be beneficial, for example if the nitrogen-fixing/symbiosis pathway is genetically engineered in a non-legume plant to enable nitrogen fixation.

### REFERENCES

1. Basosi, R., Spinelli, D., Fierro, A., & Jez, S. (2014). Mineral nitrogen fertilizers: Environmental impact of production and use. In Fertilizers: Components, Uses in Agriculture and Environmental Impacts, pp. 1-42.
2. Fowler, D., Coyle, M., Skiba, U., Sutton, M. A., Cape, J. N., Reis, S., Sheppard, L. J., Jenkins, A., Grizzetti, B., Galloway, J. N., et al. (2013). The global nitrogen cycle in the twenty-first century. Philos. Trans. R. Soc. Lond. B. Biol. Sci. 368, 20130164. Available at: https://pubmed.ncbi.nlm.nih.gov/23713126.
3. Fliegmann, J., Mithofer, A., Wanner, G., and Ebel, J. (2004). An Ancient Enzyme Domain Hidden in the Putative β-Glucan Elicitor Receptor of Soybean May Play an Active Part in the Perception of Pathogen-associated Molecular Patterns during Broad Host Resistance. J. Biol. Chem. 279, 1132-1140. Available at: http://www.jbc.org/content/279/2/1132.abstract.
4. Roy, S., Liu, W., Nandety, R. S., Crook, A., Mysore, K. S., Pislariu, C. I., Frugoli, J., Dickstein, R., and Udvardi, M. K. (2020). Celebrating 20 Years of Genetic Discoveries in Legume Nodulation and Symbiotic Nitrogen Fixation. Plant Cell 32, 15-41. Available at: https://pubmed.ncbi.nlm.nih.gov/31649123.
5. Umemoto, N., Kakitani, M., Iwamatsu, A., Yoshikawa, M., Yamaoka, N., and Ishida, I. (1997). The structure and function of a soybean β-glucan-elicitor-binding protein. Proc. Natl. Acad. Sci. 94, 1029 LP-1034. Available at: http://www.pnas.org/content/94/3/1029.abstract.

The following sequences are used in the invention (non-exhaustive list).

Sequences
MtGBP1 CDS
>MtGBP1 Medtr7g013170.1 CDS
SEQ ID NO: 1
ATGTCTTCATCATCTTCTCTTCCTTTCCTATTTCCTCAAACTCATTCAACAGTCCTCCCA
AACCCTTCAAACTTCTTCTCACAAAACCTACTATCCACACCCCTCCCTACAAACTCTTTC
TTCCAAAACTTTGTTCTCCACAATGGTGACACACCTGAATACATTCACCCTTACCTCATC
AAATCCTCAAACTTTTCCCTCTCTATTTCCTACCCTCTTCTCCTCTTTTCAGCAACAATG
TTGTACCAAGTTTTTTCACCAGATCTCACAATTTCATCCTCACAAAAATCTCACACAAAC
ACAACAAAAAACCATGTTATCTCATCATATAGTGATCTTGGTGTGACTCTTGACATTCCC
TCTTCAAATCTAAGATTCTTTTTAGTTAGAGGAAGCCCTTTTATAACTGCTTCAGTTACA
AAACCAACCTCACTTTCAATCACAACACTTCATAACATAGTTTCTTTGTCTTCTTTCGAC
GACAAAAACACCAAACATACCCTTCAACTTAATAACACTCAGAAATGGATCATATACACT
TCTTCACCAATAAAATTCAACCATGATGGTTCTGAGATTGTATCCAATCCATTTTCCGGT
ATAATCCGTATCATAGTCATTCCTAATACCAAATTTGAGAAAATTCTTGATAAATTCAGC
TCTTGTTACCCTGTCTCTGGTGATGCAAACATCAAGAATAAATTTCATTTGGAGTATAAA
TGGCAAAAGAAATGTTCTGGTGATTTACTCATGCTAGCTCACCCTCTTCATGTTAAGCTT
CTATCACAAAGTAATAATGTTAATGTTACTGTTTTGCATGATTTGAAGTATACAAGTGTC
GATGGTGATCTCGTTGGTGTTATCGGAGATTCATGGATATTGGAAACTGATCCTGTTAAT
GTAACATGGTATTCTAGTAAAGGTGTTACAAAAGAATCACATGATGAGATTGTTTCGGCT
CTTGTTAAAGATGTGAAGGAGCTGAATTCTTCAGCAATAACAACAAATGGATCTTATTTT
TATGGTAAGATTGTTTCAAGAGCTGCAAGGTTTGCATTGATAGCTGAAGAAGTATCTTAC
CCTAAAGTGATTCCAATTATCAAGAATTTTTTGAAGGAAACTATTGAGCCATGGTTGAAT
GGAACTTTCAAAGGGAATGGTTTCTTGTATGAAAAAAAATGGGGTGGATTAGTTACTAAA
CAAGGGGTTAATAATTCAGTTGTTGATTTTGGTTTTGGAATTTATAATGATCATCATTAT
CATTTAGGTTATTTTCTTTATGGAATTGCTGTTCTTGCAAAGATTGATCCATTTTGGGGA
CAAAAGTATAAACCACAAGCTTATTCACTTTTGCAAGATTTTATGAACTTGGGCCAAAGG
GATAACAAAAACTATCCAACTTTAAGGTGTTTTGATTTTTTCAAGTTGCATTCTTGGGCT
GCAGGAGTGACTGAATATGAAAATGGAAGGAATCAAGAAAGTTCAAGTGAAGCAGTGAAT
GCATATTATTCAGCAGCATTGATAGGTCTAGCATATGGCGACAAAGATCTTGTCGCCATT
GGATCAACGCTTTTAGCGTTGGAAATCAATGCTACACAAACTTGGTGGCATGTGAAAGTT
GAAAATAATTTGTATGGAGAAGAGTTTGCAAAAGAAAATAGGATTGTTGGTATTTTGTGG
GCTAATAAGAGAGATAGTAAACTTTGGTGGGCTCCTTCTGAATGTAGAGGGTGTAGGGTT
AGTATCCAAGTTATGCCTTTGTTGCCTATTACTGAGTCTTTGTTTAATGATGGTGTTTAT
GCTAAGGAGCTAGTGGAATGGACACTCCCTTCTTTGAAGAATGACACAAATGATGATAGA
TGGAAAGGGTTTATCTATTCTTTGCAAGGAATTTATGATAAAGAAAATGCATTGAAGAAG
ATTAGAATGTTGGAAGGTTTTGCTAATGGAAACTCATTCAGTAATCTCTTATGGTGGATC
CATAGCAGATGA
MsGBP1 CDS1
>M. sativa_MS.gene057477.t1_chr7.2: 84, 110,964 . . . 84, 112,955
SEQ ID NO: 2
ATGTCTTCATCATCTTCTCTTCCTTTCCTATTTCCTCAAACTCATTCAACTGTCCTCCCA
AACCCTTCAAACTTCTTCTCACAAAACCTACTATCCACACCCCTCCCTACAAACTCTTTC
TTCCAAAACTTTGTTCTCTACAATGGTGAAACACCTGAATACATTCACCCTTACCTCATC
AAATCCTCAAACTTTTCCCTATCTGTTTCATACCCTCTTCTCCTCTTTTCAACAGCAATG
TTGTACCAAGTTTTTTCACCGGATCTCACAATTTCATCCTCACAAAAAACTCACACAAAT
ATACCAAAAAACCATGTTATCTCATCATATAGTGATCTTGGTGTGACTCTTGACATTCCC
TCTTCAAACCTAAGATTCTTTTTGGTTAGAGGAAGCCCTTTTATAACTGCTTCAGTTACA
AAACCAACACCTCTTTCAATCACAACAATTCATAGTATAATTTCTTTGTCTCCTTTTGAT
AAGAAAAAAACCAAATACACCCTTCAACTCAATAACAATCAGAAATGGATCATATACACT
TCTTCACCAATCAAGTTCAACCATGATGGTTCAGAGGTTATGTCCAATCCATTTTCCGGT
ATAATTCGTATTGTCATTGTTCCTAATTCCAAATATGAGCAAGTTCTTGATAAATTCAGC
ACTTGTTACCCTGTCTCTGGTGATGCAAACATCAAGAATAAATTTCATTTGGAGTATAAA
TGGCAAAAGAAATGTTCTGGTGATTTACTCATGCTAGCTCACCCTCTTCATGTTAAGCTT
CTATCACAAAGTAATGATGCTAGTGTTACTGTTTTGCATGATTTGAAGTATACAAGTATT
GATGGTGATCTCGTTGGTGTTATCGGAGATTCATGGATATTGGAAACTAATCCTGTTAAT
GTAACATGGTATTCAAGTAAAGGTGTTACAAAAGAATCACATGATGAGATTGTTTCAGCT
CTTGTTAAAGATGTGAAGGAGCTGAATTCTTCAGCAATAACAACAAATGGATCTTATTTT
TATGGTAAGATTGTTTCAAGAGCTGCAAGGTTTGCTTTGATAGCTGAAGAAGTGTCTTAC
CCTAAAGTGATTCCAATTATCAAGAATTTTTTGAAGGAAACTATTGAGCCATGGTTGAAT
GGAACTTTCAAAGGGAATGGTTTTCTCTATGAAAAAAAGTGGGGTGGATTAGTTACTCAA
CAAGGTGTTAATGATTCAGGTGTTGATTTTGGTTTTGGAATTTATAATGATCATCATTAT
CATTTAGGGTATTTTCTTTATGGAATTGCAGTTCTTGCAAAAATTGATCCTTTTTGGGGA
CAAAAGTATAAACCTCAAACTTATGCACTTGTGAAAGATTTTATGAACTTGGGCCAAAGG
GATAACAAAAACTATCCAACTTTAAGGTGTTTTGATTTCTTCAAGTTGCATTCTTGGGCT
GCAGGAGTGACTGAATATGAAAATGGAAGGAATCAAGAAAGTTCAAGTGAAGCTGTGAAT
GCATATTATTCAGCAGCATTGATAGGTCTAGCATATGGTGACAAAGATCTTGTTGATATT
GGATCAACACTTTTAGCATTTGAAATCAATGCTACACAAACTTGGTGGCATGTGAAAGTT
GAAAAAAATTTGTATGGAGAAGAGTTTGCAAAAGAAAATAGGATTGTTGGTATTTTGTGG
GCTAATAAGAGAGATAGTAAACTTTGGTGGGCTCCTTCTGAATGTAGAGGGTGTAGGGTT
AGTATCCAAGTTATGCCTTTGTTGCCTATAACTGAGTCTTTGTTTAATGATGGTGTTTAT
GCTAAGGAGCTTGTGGAATGGACACTACCTTCTTTGAAGAATGAAACAAATGATGATAGA
TGGAAAGGGTTTATCTATGCTTTGCAAGGAATTTATGATAAAGAAAATGCATTGAAGAAG
ATTAGAATGTTGGAAAGCTTTGCTAATGGAAACTCATTCAGTAATCTCTTATGGTGGATC
CATAGCAGATAA
MsGBP1 CDS2
>M. sativa_MS.gene97210.t1_chr7.3: 84588752: 84590743
SEQ ID NO: 3
ATGTCTTCATCATCTTCTCTTCCTTTCCTATTTCCTCAAACTCATTCAACTGTCCTCCCA
AACCCTTCAAACTTCTTCTCACAAAACCTACTATCCACACCCCTCCCTACAAACTCTTTC
TTCCAAAACTTTGTTCTCCACAATGGTGAAACACCTGAATACATTCACCCTTACCTCATC
AAATCCTCAAACTTTTCCCTATCTGTTTCATACCCTCTTCTCCTCTTTTCAACAGCAATG
TTGTACCAAGTTTTTTCACCGGATCTCACAATTTCATCCTCACAAAAAACTCACACAAAT
ATACCAAAAAACCATGTTATCTCATCATATAGTGATCTTGGTGTGACTCTTGACATTCCC
TCTTCAAACCTAAGATTCTTTTTGGTTAGAGGAAGCCCTTTTATAACTGCTTCAGTTACA
AAACCAACACCTCTTTCAATCACAACAATTCATAGTATAATTTCTTTGTCTCCTTTTGAT
AAGAAAAAAACCAAATACACCCTTCAACTCAATAACAATCAGAAATGGATCATATACACT
TCTTCACCAATCAAGTTCAACCATGATGGTTCAGAGGTTATGTCCAATCCATTTTCCGGT
ATAATTCGTATTGTCATTGTTCCTAATTCCAAATATGAGCAAGTTCTTGATAAATTCAGC
ACTTGTTACCCTGTCTCTGGTGATGCAAACATCAAGAATAAATTTCATTTGGAGTATAAA
TGGCAAAAGAAATGTTCTGGTGATTTACTCATGCTAGCTCACCCTCTTCATGTTAAGCTT
CTATCACAAAGTAATGATGCTAGTGTTACTGTTTTGCATGATTTGAAGTATACAAGTATT
GATGGTGATCTCGTTGGTGTTATTGGAGATTCATGGATATTGGAAACTAATCCTGTTAAT
GTAACATGGTATTCAAGTAAAGGTGTTACAAAAGAATCACATGATGAGATTGTTTCAGCT
CTTGTTAAAGATGTGAAGGAGCTGAATTCTTCAGCAATAACAACAAATGGATCTTATTTT
TATGGTAAGATTGTTTCAAGAGCTGCAAGGTTTGCTTTGATAGCTGAAGAAGTGTCTTAC
CCTAAAGTGATTCCAATTATCAAGAATTTTTTGAAGGAAACTATTGAGCCATGGTTGAAT
GGAACTTTCAAAGGGAATGGTTTTCTCTATGAAAAAAAGTGGGGTGGATTAGTTACTCAA
CAAGGTGTTAATGATTCAGGTGTTGATTTTGGTTTTGGAATTTATAATGATCATCATTAT
CATTTAGGGTATTTTCTTTATGGAATTGCAGTTCTTGCAAAAATTGATCCTTTTTGGGGA
CAAAAGTATAAACCTCAAACTTATGCACTTGTGAAAGATTTTATGAACTTGGGCCAAAGG
GATAACAAAAACTATCCAACTTTAAGGTGTTTTGATTTCTTCAAGTTGCATTCTTGGGCT
GCAGGAGTGACTGAATATGAAAATGGAAGGAATCAAGAAAGTTCAAGTGAAGCTGTGAAT
GCATATTATTCAGCAGCATTGATAGGTCTAGCATATGGTGACAAAGATCTTGTTGATATT
GGATCAACACTTTTAGCATTTGAAATCAATGCTACACAAACTTGGTGGCATGTGAAAGTT
GAAAAAAGTTTGTATGGAGAAGATTTTGCAAAAGAAAATAGGATTGTTGGTATTTTGTGG
GCTAATAAGAGAGATAGTAGACTTTGGTGGGCTCCTTCTGAATGTAGAGGGTGTAGGCTT
AGTATACAAGTTATGCCTTTGTTGCCTATTACTGAGTCTTTGTTTAATGATGGTGTTTAT
GCTAAGGAGTTAGTGGAATGGACACTACCTTCTTTGAAGAATGAAACAAATGATGATAGA
TGGAAAGGGTTTATCTATGCTTTGCAAGGAATTTATGATAAAGAAAATGCATTGAAGAAG
ATTAGAATGTTGGAAGGTTTTGCTAATGGAAACTCATTGAGTAATCTCTTATGGTGGATC
CATAGCAGATAA
MsGBP1 CDS3
>M. sativa_MS.gene91658.t1_chr7.4: 86079832: 86081823
SEQ ID NO: 4
ATGTCTTCATCATCTTCTCTTCCTTTCCTATTTCCTCAAACTCATTCAACTGTCCTCCCA
AACCCTTCAAACTTCTTCTCACAAAACCTACTATCCACACCCCTCCCTACAAACTCTTTC
TTCCAAAATTTTGTTCTCCACAATGGTGAAACACCTGAATACATTCACCCTTACCTCATC
AAATCCTCAAACTTTTCCCTATCTGTTTCATACCCTCTTCTCCTCTTTTCAACAGCAATG
TTGTACCAAGTTTTTTCACCGGATCTCACAATTTCATCCTCACAAAAAACTCACACAAAT
ATACCAAAAAACCATGTTATCTCATCATATAGTGATCTTGGTGTGACTCTTGACATTCCC
TCTTCAAACCTAAGATTCTTTTTGGTTAGAGGAAGCCCTTTTATAACTGCTTCAGTTACA
AAACCAACACCTCTTTCAATCACAACAATTCATAGTATAATTTCTTTGTCTCCTTTTGAT
AAGAAAAAAACCAAATACACCCTTCAACTCAATAACAATCAGAAATGGATCATATACACT
TCTTCACCAATCAAGTTCAACCATGATGGTTCAGAGGTTATGTCCAATCCATTTTCCGGT
ATAATTCGTATTGTCATTGTTCCTAATTCCAAATATGAGCAAGTTCTTGATAAATTCAGC
ACTTGTTACCCTGTCTCTGGTGATGCAAACATCAAGAATAAATTTCATTTGGAGTATAAA
TGGCAAAAGAAATGTTCTGGTGATTTACTCATGCTAGCTCACCCTCTTCATGTTAAGCTT
CTATCACAAAGTAATGATGCTAGTGTTACTGTTTTGCATGATTTGAAGTATACAAGTATT
GATGGTGATCTCGTTGGTGTTATCGGAGATTCATGGATATTGGAAACTAATCCTGTTAAT
GTAACATGGTATTCAAGTAAAGGTGTTACAAAAGAATCACATGATGAGATTGTTTCAGCT
CTTGTTAAAGATGTGAAGGAGCTGAATTCTTCAGCAATAACAACAAATGGATCTTATTTT
TATGGTAAGATTGTTTCAAGAGCTGCAAGGTTTGCTTTGATAGCTGAAGAAGTGTCTTAC
CCTAAAGTGATTCCAATTATCAAGAATTTTTTGAAGGAAACTATTGAGCCATGGTTGAAT
GGAACTTTCAAAGGGAATGGTTTTCTCTATGAAAAAAAGTGGGGTGGTTTAGTTACTCAA
CAAGGTGTTAATGATTCAGGTGTTGATTTTGGTTTTGGAATTTATAATGATCATCATTAT
CATTTAGGGTATTTTCTTTATGGAATTGCAGTTCTTGCAAAAATTGATCCTTTTTGGGGA
CAAAAGTATAAACCTCAAACTTATGCACTTGTGAAAGATTTTATGAACTTGGGCCAAAGG
GATAACAAAAACTATCCAACTTTAAGGTGTTTTGATTTCTTCAAGTTGCATTCTTGGGCT
GCAGGAGTGACTGAATATGAAAATGGAAGGAATCAAGAAAGTTCAAGTGAAGCTGTGAAT
GCATATTATTCAGCAGCATTGATAGGTCTAGCATATGGCGACAAAGATCTTGTCGCCATT
GGATCAACACTTTTAGCATTTGAAATCAATGCTACACAAACTTGGTGGCATGTGAAAGTT
GAAAAATATTTGTATGGAGAAGAGTTTGCAAAAGAAAATAGGATTGTTGGTATTTTGTGG
GCTAATAAGAGAGATAATAATCTTTGGTGGGCTCCTTCTGAATGTAGAGGGTGTAGGCTT
AGTATACAAGTTATGCCTTTGTTGCCTATTACTGAGTCTTTGTTTAATGATGGTGTTTAT
GCTAAGGAGCTAGTGGAATGGACATTTCCTTCTTTGAAGAATGAAACAAATGATGATAGA
TGGAAAGGGTTTATCTATGCTTTGCAAGGAATTTATGATAAAGAAAATGCATTGAAGAAG
ATTAGAATGTTGGAAGGTTTTGCTAATGGAAACTCATTCAGTAATCTCTTATGGTGGATC
CATAGCAGATAA
MsGBP1 CDS4
>M. sativa_MS.gene021861.t1_chr7.1: 82777978: 82779969
SEQ ID NO: 5
ATGTCTTCATCATCTTCTCTTCCTTTCCTATTTCCTCAAACTCATTCAACTGTCCTCCCA
AACCCTTCAAACTTCTTCTCACAAAACCTACTATCCACACCCCTCCCTACAAACTCTTTC
TTCCAAAACTTTGTTCTCCACAATGGTGAAACACCTGAATACATTCACCCTTACCTCATC
AAATCCTCAAACTTTTCACTCTCTGTTTCCTACCCTCTTCTCCTCTTTTCAGCAACAATG
TTGTACCAAGTTTTTTCACCGGATCTCACAATTTCATCCTCACAAAAAACTCACACAAAT
ATACCAAAAAATCATGTTATCTCATCACATAGTGATCTTGGTGTGACTCTTGACATTCCC
TCTTCAAATCTAAGATTCTTTTTGGTTAGAGGAAGCCCTTTTATAACTGCTTCAGTTAGA
AAACCAACCTCACTTTCAATCACAACACTTCATAACATAGTTTCTTTGTCTTCTTTTGAT
GACAAAAATACCAAATACACCCTTCACCTCAACAACACTCAGCAATGGATCATATACACT
TCTTCACCTATAAAATTCAACCATGATGGTTCTGAGATTGTATCCAATCCATTTTCCGGT
ATAATTCATATCGTAGTTGTTCCTAGTTCCAAATATGAGAAAATTCTTGATAAATTGAGC
TCTTGTTACCCTGTCTCCGGTGATGCAAACATCAAGAATAGATTTCATTTGGAGTATAAA
TGGAAAAAGAAATGTTCTGGAGATTTACTCATGCTAGCACACCCTCTTCATGTTAAGCTT
CTATCACAAAGTAACAATGTTAATGTTACTGTTTTGCATGATTTGAAGTATACAAGTGTT
GATGGTGATCTCGTTGGTGTTATCGGAGATTCATGGATATTGAAAACTGATCCTGTTAAT
GTAACATGGTATTCTAGTAAAGGTGTTACAAAAGAATCACATGATGAGATTGTTTCAGCT
CTTGTTAACGATGTGAAAGAGCTGAATTCTTCAGCAATAACAACAAATGGATCTTATTTT
TATGGTAAGATAGTTTCAAGAGCTGCAAGGTTTGCTTTGATAGCTGAAGAAGTTTCTTAC
CCTAAAGTGATTCCAATTATCAAGAATTTTTTGAAGGAAACTATTGAGCTATGGTTGAAT
GGAACTTTCAAAGGGAATGGTTTTTTGTATGAAAAAAAATGGGGTGGATTAGTTACTAAA
CAAGGGGTTAATAATTCAGGTGTTGATTTTGGTTTTGGAATTTATAATGATCATCATTAT
CATTTAGGGTATTTTCTTTATGGAATTGCAGTTCTTGCAAAGATTGATCCATTTTGGGGA
CAAAAGTATAAACCACAAATTTATGCACTTGTGAAAGATTTTATGAACTTGGGCCAAAGG
GATAACAAAAACTATCCAACTTTAAGGTGTTTTGATTTTTTCAAGTTGCATTCTTGGGCT
GCAGGAGTGACTGAATATGAAAATGGAAGGAATCAAGAAAGTTCAAGTGAAGCTGTGAAT
GCATATTATTCAGCAGCATTGATAGGTCTAGCATATGGTGACAAAGATCTTGTTGCTATT
GGATCAACACTTTTAGCATTTGAAATCAATGCTACACAAACTTGGTGGCATGTGAAAGTT
GAAAATAATTTATATGGAGAAGAGTTTGCAAAAGAAAATAGGATTGTTGGTATTTTGTGG
GCTAATAAGAGAGATAGTAAACTTTGGTGGGCTCCTTCTGAATGTAGAGGGTGTAGGGTT
AGTATCCAAGTTATGCCTTTGTTGCCTATTACTGAGACATTGTTCAATGATGGTGTTTAT
GCTAAGGAATTAGTGGAATGGACACTACCTTCTTTGAAGAATGAAACAAATGATGATAGA
TGGAAAGGGTTTATCTATGCTTTGCAAGGAATTTATGATAAAGGAAATGCATTGAAGAAT
ATTAGAATGTTGGAAGGTTTTGCTAATGGAAACTCATTCAGTAATCTCTTATGGTGGATT
CATAGCAGATAA
MsGBP1 CDS5
>M. sativa_MS.gene069419.t1_chr7.2: 83500785: 83502767
SEQ ID NO: 6
ATGTCTTCTGTTCCATTCCTATTTCCTCAGACTCATTCAACTGTCCTCCCAAACCCTTCA
AACTTCTTCTCACAAAACTTACTATCCACACCCCTCCCTACAAATTCATTCTTCCAAAAC
TTTGTTCTCCAAAATGGTGATCAACATGAATACATTCACCCTTACCTTGTCAAATCCTCA
AACTTTTCCGTATCTGTTTCATACCCTCTTCTCCTCTTTTCAACAGCAATGTTGTACCAA
GTTTTTTCACCAGATCTTACAATCTCATCCTCACAAAAAACTCACACAAACATACCTAAA
AACCATGTTATTTCATCATATAGTGATCTTGGTGTGACTCTTGACATTCCCTCTTCAAAC
CTAAGATTCTTTTTGGTTAGAGGAAGCCCTTTTATAACTGCTTCAGTTACAAAACCAACA
CCTCTTTCAATCACAACAATTCATAGTATAATTTCTTTGTCTCCTTTTGATAAGAAAAAA
ACCAAATATACTCTTCAACTCAACAACAATCAGACATGGATCATATACACTTCTTCACCA
ATCAACTTGAACCATGATGGTTCCGAGGTTAAGTCCGGTCCATTTTCCGGTATTATTCGT
ATCGCGGTTGTTCCTGATTCCAATGGTGAGAAAATTCTTGATAAATTCAGCTCTTGTTAC
CCTGTCTCTGGTGATGCAAACATCAAGAAGAAATTTGGTTTGGTTTATAAATGGCAAAGG
AAAAATTCTGGTGATTTACTCATGCTAGCACACCCTCTTCATGTTAAGCTTTTATCAAAA
AGTAACAATCATGGTGTTACTGTTCTTGATGATTTTAAGTATAAGAGTGTTGATGGTGAT
CTTGTTGGTGTTGTTGGAAATTCATGGAATTTGAAAACTGATTCTGTTAATGTAACATGG
CATTCAAACAAAGGTGTTACAAAAGAATCACATGCTGAAATTGTTTCTGCTCTTGTTAAT
GATGTGAAGAAGCTAAACTTTTCGTCGATAACAACAAATTCATCTTATTTTTATGGTAAG
ATTGTTGGAAGAGCTGCAAGGTTTGCTTTCATAGCTGAAGAAGTTTCTTACCCTAAAGTG
ATTCCAATTATCAAGAATTTTTTGAAGGAAACTATTGAGCCATGGTTGGATGGAAATTTC
AAAGGGAATGGTTTTTTCTATGAAAAAAGTTGGGGTGGATTTGTTACTCAACAAGGGATT
AATGATTCAAGTGCTGATTTTGGTTTTGGAATTTATAATGATCATCATTATCATTTAGGT
TATTTTCTTTATGGAATTGGAGTTCTTGCAAAAATTGATCCATCTTGGGGACAAAAGTAT
AAACCTCAAGTTTATTCACTTGTGAAAGATTTTATGAACTTGGGCCAAAGGGATAACAAA
AATTATCCAACTTTAAGGTGTTTTGATCCATACAAGTTGCATTCTTGGGCTTCGGGGTTG
ACCGAATTTGAACATGGAAGGAATCAAGAAAGTTCGAGCGAAGCTGTGAATGCGTACTAT
TCAGTAGCATTGGTTGGTTTGGCATATGGCGACAAAGATCTTGTCGCCACTGGATCAACG
CTTTTAGCGTTGGAAATTAATGCCGTGCAAACTTGGTGGCATGTGAAATTCGAAAATAAT
TTGTATGGTGGAGATTTCGCAAAAGGGAATCGGATAGTGGGAATTTTATGGTCAAACAAA
AGAGATAGTGCATTATGGTGGGCTGCATCTGAATGTAGAGAGTGTAGGCTTAGTATACAA
GTTTTGCCTTTGTTGCCTATAACTGAGTCTTTGTTCAATGATGGTGTTTATGCTAAGGAG
CTTGTGGAATGGACAGTGCCTTCTTTCAAGAACAAGACTAATATTGAAGGGTGGAAAGGG
TTTACCTATGCTTTGCAAGGTGTTTATGATAAGAAGAATGCATTGAAGAATATTAGAATG
TTGAAAGGTTTTGATGATGGTAACTCTTTTAGTAATATGTTATGGTGGATTCATAGTAGG
TAA
MsGBP1 CDS6
>M. sativa_MS.gene021900.t1_chr7.1: 82129837: 82131819
SEQ ID NO: 7
ATGTCTTCTGTTCCATTCCTATTTCCTCAAACTCATTCAACTGTCCTCCCAAACCCTTCA
AACTTCTTCTCACAAAACTTACTATCCACACCCCTCCCTACAAATTCATTCTTCCAAAAC
TTTGTTCTCCAAAATGGTGATCAACATGAATACATTCACCCTTACCTTGTCAAATCCTCA
AACTCTTCCCTATCTGTTTCATACCCTCTTCTCCTCTTTTCAACAGCAATGTTGTACCAA
GTTTTTTCACCAGATCTTACAATCTCATCCTCACAAAAAACTCACACAAACATACCTAAA
AACCATGTTATTTCATCATATAGTGATCTTGGTGTGACTCTTGACATTCCCTCTTCAAAC
CTAAGATTCTTTTTGGTTAGAGGAAGCCCTTTTATAACTGCTTCAGTTACAAAACCAACA
CCTCTTTCAATCACAACAATTCATAGTATAATTTCTTTGTCTCCTTTTGATAAGAAAAAA
ACCAAATATACTCTTCAACTCAACAACAATCAGACATGGATCATATACACTTCTTCACCA
ATCAACTTGAACCATGATGGTTCCGAGGTTAAGTCCGGTCCATTTTCCGGTATTATTCGT
ATCGCGGTTGTTCCTGATTCCAATGGTGAGAAAATTCTTGATAAATTCAGCTCTTGTTAC
CCTATTTCTGGTGATGCAAACATCAAGAAGAAATTTGGTTTGGTTTATAAATGGCAAAGA
AAAAACTCTGGTGATTTACTCATGCTTGCACACCCTCTTCATGTTAAGCTTTTATCAAAA
AGTAACAATCATGGTGTTACTGTTCTTGATGATTTCAAGTATAAAAGTGTTGATGGTGAT
CTTGTTGGTGTTGTTGGAAATTCATGGAATTTGAAAACTGATTCTGTTAATGTAACATGG
CATTCAAATAAAGGTGTTACAAAAGAATCACATGCTGAAATTGTTTCTGCTCTTCTTAAT
GATGTTAAGAAGTTAAACATTTCGTCGATAACAACAAATTCATCTTATTTTTATGGCAAG
ATTGTTGGAAGAGCTGCAAGGTTTGCTTTAATAGCTGAAGAAGTTTCTTACCCTAAAGTG
ATTCCAATAATCAAGAATTTTTTGAAGGAGACTATTGAGCCATGGTTGGATGGAAATTTC
AAAGGGAATGGTTTTTTCTATGAAAAAAGTTGGGGTGGATTAGTTACTCAACAAGGGATT
AATGATTCAAGTGCTGATTTTGGTTTTGGAATTTATAATGATCATCATTATCATTTAGGT
TATTTTCTTTATGGAATTGGAGTTCTTGCAAAAATTGATCCTTCTTGGGGACAAAAGTAT
AAACCACAAGTTTATTCACTTGTGAAAGATTTTATGAACTTGGGCCAAAGGGATAACAAA
AATTATCCAACTTTAAGGTGTTTTGATCCATACAAGTTGCATTCTTGGGCTTCGGGGTTG
ACCGAATTTGAACATGGAAGGAATCAAGAAAGTTCGAGTGAAGCTGTGAATGCGTACTAT
TCAGTAGCATTGGTTGGTTTAGCATATGGCGACAAAGATCTTGTCGCCACTGGATCAACG
CTTTTAGCGTTGGAAATTAATGCCGTGCAAACTTGGTGGCATGTGAAAGTCGAAAATAAT
TTATATGGTCAAGATTTCGCGAAAGAGAATCGGATAGTGGGAATTTTGTGGGCTAACAAA
AGAGATAGTGCACTATGGTGGGCTGCATCTGAATGTAGAGAGTGTAGGCTTAGTATACAA
GTTTTGCCTTTGTTGCCTATAACTGAGTCTTTGTTCAATGATGGTGTTTATGCTAAGGAG
CTTGTGGAATGGACAGTGCCTTCTTTCAAGAACAAAACTAATATTGAAGGTTGGAAAGGG
TTTACCTATGCTTTGCAAGGTGTTTATGATAAGAAGAATGCATTGAAGAATATTAGAATG
TTGAAAGGTTTTGATGATGGAAACTCTTTTAGTAATATGTTATGGTGGATTCATAGTAGG
TGA
MsGBP1 CDS7
>M. sativa_MS.gene91618.t1_chr7.4: 85331421: 85333175
SEQ ID NO: 8
ATGTTATACCAAGTTTTTTCACCAGATGTTACAATTTCTTCCTCACAAAAAACTCACACA
AACATACCAAAAAACCATGTTATCTCATCATATAGTGATCTTGGTGTGACTCTTGACATT
CCCTCTTCAAACCTAAGATTCTTTTTGGTTAGAGGAAGCCCTTTTATAACTGCTTCAGTT
ACAAAACCAACACCTCTTTCAATCACAACAATTCATAGTATAATATCTTTGTCTCCTTTT
GATAAGAAAAAAACCAAATATACCCTTCAACTCAATAACAATCAGACATGGATCATATAC
ACTTCTTCACCAATCAACTTCAACCATGATGGTTCCGAGGTTAAATCCGGTCCATTTTCC
GGTATAATTCGTATCGCGGTTGTTCCTGATTCCAATGGTGAGAAAATTCTTGATAAATTC
AGCTCTTGTTATCCTGTTTCTGGTGATGCAAACATCAAGAAGAAATTTGGTTTGGTTTAT
AAATGGCAAAGAAAAAATTCTGGTGATTTACTCATGCTAGCACACCCTCTTCATGTTAAG
CTTTTATCAAAAAGTAACAATCATGGTGTTACTGTTCTTGATGATTTCAAGTATAAAAGT
GTTGATGGTGATCTTGTTGGTGTTGTTGGAAATTCATGGAATTTGAAAACTGATTCTGTT
AATGTAACATGGCATTCAAACAAAGGTGTTACAAAAGAATCACATGCTGAAATTGTTTCT
GCTCTTGTTAATGATGTGAAGAAGTTAAACTTTTCGTCGATAACAACAAATTCATCTTAT
TTTTATGGTAAGATTGTTGGAAGAGCTGCAAGGTTTGCTTTAATAGCTGAAGAAGTTTCT
TACCCTAAAGTGATTCCAATTATCAAGAATTTTTTGAAGGAGACTATTGAGCCATGGTTG
GATGGAAATTTCAAAGGGAATGGTTTTTTCTATGAAAAAAGTTGGGGTGGATTAGTTACT
CAACAAGGGATTAATGATTCAAGTGCTGATTTTGGTTTTGGAATTTATAATGATCATCAT
TATCATTTAGGTTATTTTCTTTATGGAATTGGAGTTCTTGCAAAAATTGATCCTTCTTGG
GGACAAAAGTATAAGCCACAAGTTTATTCACTTGTGAAAGATTTTATGAACTTGGGCCAA
AGGGATAACAAAAATTATCCAACTTTAAGGTGTTTTGATCCATACAAGTTGCATTCTTGG
GCTTCGGGGTTGACCGAATTTGAACATGGAAGGAATCAAGAAAGTTCGAGCGAAGCTGTG
AATGCGTACTATTCAGTAGCATTGGTTGGTTTAGCATATGGCGACAAAGATCTTGTCGCC
ACTGGATCAACGCTTTTAGCGTTGGAAATTAATGCCGTGCAAACTTGGTGGCATGTGAAA
GTCGAAAATAATTTATATGGTCAAGATTTTGCGAAAGAGAATCGGATAGTGGGAATTTTG
TGGGCAAACAAAAGAGATAGTGCACTATGGTGGGCTTCATCTGAATGTAGAGAGTGTAGG
CTTAGTATACAAGTTTTGCCTTTGTTGCCTATAACTGAGTCTTTGTTCAATGATGGTGTT
TATGCTAAGGAGCTTGTGGAATGGACAGTGCCTTCTTTCAAGAACAAAACTAATATTGAA
GGTTGGAAAGGGTTTACCTATGCTTTGCAAGGTGTTTATGATAAGAAGAATGCATTGAAG
AATATTAGAATGTTGAAAGGTTTTGATGATGGAAACTCTTTTAGTAATATGTTATGGTGG
ATTCATAGTAGGTGA
MsGBP1 CDS8
>M. sativa_MS.gene44625.t1_chr7.3: 83746893: 83748647
SEQ ID NO: 9
ATGTTATACCAAGTTTTTTCACCAGATCTTACAATCTCATCCTCACAAAAAACTCACACA
AACATACCTAAAAACCATGTTATTTCATCATATAGTGATCTTGGTGTGACTCTTGACATT
CCCTCTTCAAACCTAAGATTCTTTTTGGTTAGAGGAAGCCCTTTTATAACTGCTTCAGTT
ACAAAACCAACACCTCTTTTGATCACAACAATTCATAGTATAATTTCTCTGTCTCCTTTT
GATAAGAAAAAAACCAAATACACCCTTCAACTCAATAACAATCAGACATGGATCATATAC
ACTTCTTCACCAATCAACTTCAACCATGATGGTTCTGAGGTTAAATCCGGTCCATTTTCC
GGTATTATTCGTATCGCGGTTGTTCCTGATTCCAATGGTGAGAAAATTCTTGATAAATTC
AGCTCTTGTTACCCTATTTCTGGTGATGCAAACATCAAGAAGAAATTTGGTTTGGTTTAT
AAATGGCAAAGAAAAAATTCTGGTGATTTACTCATGCTAGCACACCCTCTTCATGTTAAG
CTTTTATCAAAAAGTAACAATCATGGTGTTATTGTTCTTGATGATTTTAAGTATAAAAGT
GTTGATGGTGATCTTGTTGGTGTTGTTGGAAATTCATGGAATTTGAAAACTGATTCTGTT
AATGTAACATGGCATTCAAACAAAGGTGTTACAAAAGAATCACATGCTGAAATTGTTTCT
GCTCTTGTTAATGATGTGAAGAAGCTAAACTTTTCGTCGATAACAACAAATTCATCTTAT
TTTTATGGTAAGATTGTTGGAAGAGCTGCAAGGTTTGCTTTAATAGCTGAAGAAGTTTCT
TACCCTAAAGTGATTCCAATTATCAAGAATTTTTTGAAGGAAACTATTGAGCCATGGTTG
GATGGAAATTTCAAAGGGAATGGTTTTTTCTATGAAAAAAGTTGGGGTGGATTAGTTACT
CAACAAGGGATTAATGATTCAAGTGCTGATTTTGGTTTTGGAATTTATAATGATCATCAT
TATCATTTAGGGTATTTTCTTTATGGAATTGGAGTTCTTGCAAAAATTGATCCTTCTTGG
GGACAAAAGTATAAACCACAAGTTTATTCACTTGTGAAAGATTTTATGAACTTGGGCCAA
AGGGATAACATAAATTATCCAACTTTAAGGAGTTTTGATCCATACAAGTTGCATTCTTGG
GCTTCGGGGTTGACCGAATTTGAACATGGAAGGAATCAAGAAAGTTCGAGTGAAGCTGTG
AATGCGTACTATTCAGTAGCATTGGTTGGTTTAGCATATGGCGACAAAGATCTTGTCGCC
ACTGGATCAACGCTTTTAGCGTTGGAAATTAATGCCGTGCAAACTTGGTGGCATGTGAAA
GTCGAAAATAATTTATATGGTCAAGATTTCGCGAAAGAGAATCGGATAGTGGGAATTTTG
TGGGCAAACAAAAGAGATAGTGCACTATGGTGGGCTTCATCTGAATGTAGAGAGTGTAGG
CTTAGTATACAAGTTTTGCCTTTGTTGCCTGTAACTGAGTCTTTGTTCAATGATGGTGTT
TATGCTAAGGAGCTTGTGGAGTGGACAGTGCCTTCTTTCAAGAACAAAACTAATATTGAA
GGTTGGAAAGGGTTTACCTATGCTTTGCAAGGTGTTTATGATAAGAAGAATGCATTGAAG
AATATTAGAATGTTGAAAGGTTTTGATGATGGTAACTCTTTTAGTAATTTGTTATGGTGG
ATTCATAGTAGGTAA
PsGBP1 CDS1
>Psat3g201680.1
SEQ ID NO: 10
ATGTCTTCTCCTCCTTACCTATTTCCTCAAACTCAATCAACCATTCTCCCAAACCCTTCC
AACTTCTTCTCCCCAAACTTGCTATCCACACCCCTCCCTACCAGCTCTTTCTTCCAAAAC
TTTGCTCTAAAAAATGGCGACCAACCTGAATACATTCACCCTTACCTCATCCAATCCTCA
AACTCTTCACTCTCGGTTTCATACCCTTTACTCCTCTTCTCCACAGCATTACTCTACCAG
GTTTTCTCACCAGATCTCACTATCTCATCCACACAAAAACCTCAAACAAACATACCACAA
AACAACCATGTTATATCCTCGTACAGTGATCTTGGTGTGACGCTTGATATTCCCACTGCA
AATCTAAGATTTTTTCTAGTTAGAGGAAGCCCTTTTGTAACTGCTTTAGTTACAAAACCA
ACACCTCTTTCAATCAAAACAAATCACACCATTGTTTCTTTCTCATCATTTGATTACAAG
AAAACCAAATATAGACTATCACTCAACAATGGTCAGAAATGGATCATATACACTTCTTCA
CCAATTAACTTCAACCATGACGGTTCAGAGGTTAAGTCCGATCCGTTTTCTGGTATAATC
CGTTTCGCCGTTGTTTCTAATTCGAATAATGAGAAAATTCTCCATGAATTCAGCTCGAGT
TACCCCGTTTCCGGCTATGCAAAGATCGAGGATAAATTCGGTTTGGTTTATAAATGGAAA
ACTAAAAATTCCGGTGATTTACTCATGCTAGCACATCCTCTTCATGTTAAGCTTTTGTCG
AAGAATAGTAACGATCATAAAGTTACTATTTTGAATGATTTTAAGTATAGAAGCGTTGAT
GGTGATCTTGTTGGTGTTGTTGGAAAATCATGGTTGTTGAAAACTGATTCTGTTAATGTA
ACATGGCATTCTAGTAAAGGTGTTTCAAAAGATTCATACGAGGAAGTTGTTTCTGCTCTT
GAGAAAGATGTGAATGAGTTGAACGTTGCGACGATAAATACAACTTCGTCTTATTTTTAC
GGCAAGATTGTTGCAAGAGCTGCAAGGCTTGCTTTGATAGCTGAAGAAGTTTCTTATGAG
AAAGTGATTCCAATTGTTAAGGATTTTTTGAAGAAAACTATTGAGCCATGGTTAGATGGA
AACTTCAAAGGGAATGGTTTTTTGTATGAGAAAACATGGGGTGGATTGGTTACTCAACAA
GGGGTTAATGATAGTGGTGCTGATTTTGGTTTTGGTGTTTATAACGATCATCATTATCAT
TTAGGTTATTTTCTTTATGGAATTGGAGTTCTTGCAAAACTTGATCAAGATTGGGGACAA
AAGTATAAACCAATAGTTTATTCACTTTTGAAAGATTTTATGAACTTGGGTCAAAGGGAT
AACAAAAACTATCCAACTTTAAGGAGTTTTGATCCTTACAAGTTACATTCTTGGGCTTCG
GGGTTGACCGAATTCAGAGACGGAAGGAATCAAGAAAGTACAAGCGAAGCTGTGAACGCG
TACTACTCGGTTACCTTAGTAGGTTTAGCTTATGACGATGAAGATTTGGTCGCGATCGGA
TCGACGCTTTTAGCGTTCGAAATTAACGCGGCGCAAACTTGGTGGCACGTGAAAGCCGAG
AACAATGTGTATGGTACTGATTTTGCTAAGCAAAATCCGGTAGTTGGTGTTTTGTGGGCG
AACAAGAGAGATAGTAGTCTTTGGTGGGCTTCGTCGGAGTGTCGCCAGTGTCGGCTTAGT
ATACAAGTTTTGCCTTTGTTGCCTATAACTGAGAATTTGTTCAATGATGGTGTTTATGCT
AAGGAGCTTGTGGAATGGACATGGCCAACTTTGAGTAAAGAAGGGTGGAAAGGGTTTACT
TATGCTTTGCAAGGTGTTTATGATAAGGAAAATGCTTTGAAGAATATTAGAACTTTGAAA
GGTTTTGATGATGGAAACTCTTTGAGTAATTTGTTATGGTGGATTCATAGTAGATGA
PsGBP1 CDS2
>Psat3g201640
SEQ ID NO: 11
ATGTGTTCTCCTCCTTACCTATTTCCTCAAACTCAATCAACCATTCTCCCAAACCCTTCC
AACTTCTTCTCCCAAGACTTACTATCCACACCCCTACCTACAAACTCTTTCTTCCAAAAC
TTTGCTCTCAAAAATGGTGACCAACCTGAATACATTCACCCTTACCTCATCCAATCCTCA
AACTCTTCACTCTCGGTTTCATTCCCTCTGCTCTTCTTCTCCACAGCATTGCTCTACCAG
GTTTTCACACCAGATCTCACTATCTCATCCACACAAAAACCTCAAACAAACATACCACAA
AACAACCATGTTATATCCTCGTACAGTGATCTTGGTGTGACGCTTGATATTCCCACTACA
AATCTAAGATTTTTTTTGGTTAGAGGAAGCCCTTTTGTAACAGCTCAAGTTACAAAACCA
ACACCTCTTTCAATCAAAACAATTCACGCCATTCTTTCTTTCTCATCATTTGATAACAAA
AAAAACAAATATGCACTTTCACTCAACAATGGTCAGAAATGGATCATATACACTTCTTCA
CCAATTAACTTCAACCATGATATTTCCGAGGTTAAATCCGATCCGTTTACCGGTGTAATC
CGTATCGCAGCTGTTTCTGATTCGAATAACGAGAAAATTCTCGACGAATTCAGCTCGAGT
TATCCCGTTTCCGGCCATGCAATTGTGGACGTAAAGAATAAATTTGGTTTGGTTTATAAA
TGGGAGACTGAAAATTCCGGTGATTTACTCATGTTAGCACACCCTCTTCATGTCAAGCTT
TTGTCGAAGAATAGTAACGATCATAAAGTTACTATTTTGAATGATTTTAAGTATAGAAGC
GTTGATGGTGATCTTGTTGGTGTTGTTGGAAATTCATGGTTGTTGAAAACTGATACTATT
AATGTAACATGGCATTCTAGTAAAGGTGTTGCAAAAGAATCATATGAGGAAGTTGTTTCT
GCTCTTGAGAAAGATGTGAATGAGTTGAACGTTGCGTCGATAAGTACGACTTCGTCTTAT
TTTTACGGTAAGATTGTTGCAAGAGCTGCAAGGTTTGCTTTGATAGCTGAAGAAGTTTCT
TATGAGAAAGTGATTCCAATTGTTAAGGATTTTTTGAAGAAAACTATTGAGCCATGGTTA
GATGGAAACTTCAAAGGGAATGGTTTTTTGTATGAGAAAACATGGGGTGGATTGGTTACT
CAACAAGGGGTTAATGATGCTGGTGCTGATTTTGGTTTTGGTGTTTATAATGATCATCAT
TATCATTTAGGTTATTTTCTTTATGGAGTTGGAGTTCTTGCAAAACTTGATCAAGATTGG
GGACAAAAGTATAAGCCAATAGTTTATTCCCTTTTGAAAGATTTTATGAACTTGGGTCAA
GGGGATAACAAAAATTATCCAACTTTAAGGAGTTTTGATCCTTACAAGTTGCATTCTTGG
GCTTCCGGGTTGACCGAATTCAGTGATGGAAGGAATCAAGAAAGTACGAGTGAAGCTGTG
AATGCGTATTATTCAGCTGCATTGGTAGGTTTAGCTTATGGTGACGAAGATCTGGTTGCG
ATTGGATCGACGCTTTTAGCATTGGAAATTAACGCGGCACAAACTTGGTGGCATGTGAAA
ACCGAGAATAATGTGTATGGTGCCGATTTTGCTAAACAAAATTCGGTAGTTGGTGTTTTG
TGGGCGAACAAGAGAGATAGTAGTCTTTGGTGGGCTTCTTCGGAATGTCGCGAATGTCGA
CTTAGTATACAAGTTTTGCCTTTGTTGCCTATAACTGAGAATTTGTTCAATGATGGTGTT
TATGTTAAGGAGCTTGTGGAGTGGACATGGCCAACTTTGAGTAATGAAGGGTGGAAAGGG
TTTACTTATGCTTTGCAAGGTGTTTATGATAAGGAAAATGCTTTAAATAATATTAGAGCT
TTGAAAGGTTTTGATGATGGAAATTCTTTGAGTAATATTTTATGGTGGATTCATAGTAGA
TGA
VfGBP1 CDS
>V. faba_jg123098.t1
SEQ ID NO: 12
ATGTCTTCTCCTCCTTACCTATTTCCTCAAACTCAATCAACCATTCTCCCAAACCCTTCC
AACTTCTTCTCCCAAAACTTGCTATCCACACCCCTCCCTACAAACTCTTTCTTCCAAAAC
TTTGCTCTCAAAAATGGTGACCAACCTGAATACATTCACCCTTACCTCATCCAATCCTCA
AACTCCTCCCTCTCAGTTTCATACCCTTTACTCCTCTTCTCAACAGCATTGCTCTACCAG
GTTTTCTCACCAGATCTCACTATCTCATCCACACAACAACCTCAAACAAACATAAACCAT
GTTATATCCTCGTACAGTGATCTTGGTGTGACTCTTGATATTCCCACTTCGAATCTACGA
TTTTTTCTCGTTAGAGGAAGTCCTTTTGTAACTGCTCTAGTCACAAAACCAACACCTCTT
TCAATCAAAACTATTCACACCATTGTTTCTTTCTCTACATTCGATAACAAGAAAACCAAA
TATACACTTTCACTCAACAATACTCAGAAATGGATCATATACACTTCTTCACCAATTAAC
TTCAACCATCTCGGTTCCGAGGTTATATCCGATCCATTTTCCGGTATAATTCGTATTGCA
AGTGTTTCTAATTCGAATAATGAGAAAATTCTCGATGAATTCAGCTCGAGTTATCCGGTT
TCGGGCTATGCGAAGATCGAGAATAAATTCGGTTTAGTTTATAAATGGGAGACTCAAAAT
TCCGGTGATTTACTCATGCTAGCACACCCTCTTCATGCTAAGCTTTTGTCTAATAGTAAA
GATCATAAGGTTACTATTTTGAACGATTTTAAGTATAGAAGCATTGATGGTGATCTTGTT
GGTGTTGTTGGAAATTCATGGTTGTTGAAAACCGATTCTTTTAATGTAACATGGCATTCT
AGTAAAGGTGTTACAAAAGAATCATACGAGGAAGTTGTTTCTGCTCTTGAGAAAGATGTT
AATGAGTTGAATGTTGCGTCGATTACGACGACTTCGTCGTATTTTTATGGTAAGATTGTT
GCAAGAGCTGCAAGGTTTGCTTTGATAGCTGAAGAAGTTTCTTATGAGAAAGTGATTCCG
GTTGTTAAGGGTTTTTTGAAGCAAACTATTGAGCCATGGTTAGATGGAAAGTTCAAAGGG
AATGGTTTTTTGTATGAGAAAACTTGGGGTGGATTGGTTACTCAACAAGGGGTGAATGAT
GTTGGTGCTGATTTTGGTTTTGGTGTTTATAATGATCATCATTATCATTTAGGTTATTTT
CTTTATGGAATTGGAGTTCTTGCAAAGATTGATCAAGATTGGGGACAAAAGTATAAGCCA
ATAGTTTATTCACTTTTGAAAGATTTTATGAACTTGGGTCTAGGGGATAATCCAAACTAT
CCAACTTTAAGGAATTTTGATCCTTACAAGTTACATTCTTGGGCTTCGGGGTTGACCGAA
TTCAGAGACGGAAGGAATCAAGAAAGTACGAGTGAAGCTGTGAATGCGTATTATTCAGTT
ACGTTAATAGGTTTAGCTTATGGTGACGAAGATCTGGTTGTGGTTGGATCGACACTTTTA
GCGTTGGAAATTAACGCGGCGCAATCTTGGTGGCATGTGAAAGCCGAGAACAATGTGTAT
GGTACTGATTTTGCTAAACAAAATCCGATTGTCGGAGTTTTGTGGGCGAACAAGAGAGAC
AGTAGTCTTTGGTGGGCTTCGTCGGCGTGCCGTGAATGTCGGCTTAGTATACAAGTTTTG
CCATTGTTGCCTATCACTGAGAATTTGTTCAATGATGGTGTTTATGCTAAGGAGCTTGTG
GAATGGACATTGCCAACTTTGAGTAATGAAGGGTGGAAAGGGTTTACTTATGCTTTGCAA
GGTGTTTATGATAAGGAAAATGCATTGAAGAATATTAGAACTTTGAAAGGTTTTGATGAT
GGAAACTCTTTGAGTAATTTGTTATGGTGGATTCATAGTAGATGA
TpGBP1 CDS
>T. pratense_Tp57577_TGAC_v2_mRNA26446
SEQ ID NO: 13
ATGTCTTCTGTTCCTTTCCTATTTCCTCAAACTCATTCAACTGTTCTTCCAAACCCTTCA
AATTTCTTCTCACAAAACTTACTATCCACACCCCTCCCTACTAACTCTTTCTTCCAAAAC
TTTGTTCTCAAAAATGGTGACCAACCTGAATACATTCACCCTTATCTCATCAAATCCTCA
AACTTTTCACTTTCTGTTTCATACCCTTTTCTCCTATTTTCAACAGCAATGTTGTACCAA
GTTTTCTCACCAGATCTCACTATTTCATCCTCACAAAAATCTCACACAAACTCACAAAAA
AATAAGCATTTTATCTCATCCTATAGTGATCTTGGCGTGACTCTTGATATTCCATCTTCA
AATCTAAGATTCTTTCTTGTTAGAGGAAGTCCTTTTGTAACTGCTTCTGTTACAAAACCA
ACACCTCTTTCAATCACAACATTGCATAACATAGTTTCTTTGTCTTGTTTTGATAACAAA
AAAACCAAATATACACTTTTGCTCAACAATACTCAGAAATGGATTATATACACTTCTTCA
CCAATCAATTTAAACCATGATGGTTCCGAGGTGAAATCCGGTCCATTTTCGGGGATAATT
CGTATCGCGGTTGTTCCTGATTCGAATTACGAGAAGATTCTCGATAAATTCAGCTCTTGT
TACCCTGTTTCTGGCTATGCAAACATTCAGAAGAAATTTGGTTTGGTTTATAAATGGCAA
AGGAAAAATTCAGGTGATTTACTTATGCTAGCACACCCTCTTCATGTTAAGCTTTTATCA
AAAAGTAACAATCATGGTGTTACTGTTTTGAATGATTTTAAGTATAGAAGTGTTGATGGT
GATCTTGTTGGTGTGGTTGGAAATTCATGGAATTTGAAAACTGATCCTATTGATGTAACA
TGGCATTCTAGTAAAGGTGTTACAAAAGAATCACATGATGAAATTGTTTCAGCACTTGTT
AAAGATGTGAAGAAATTGAATATTTCAGCAATAGAAACAAATTCATCTTATTTTTATGGT
AAGATTGTTGGAAGAGCTGCAAGATTTGCTTTGATAGCTGAAGAAATTTCTTATTTTAAA
GTGATTCCAATTATTAAGAATTTTTTGAAGAAAACTATTGAGCCATGGTTAGATGGTAAT
TTCAAAGGGAATGGTTTTTTTTATGAAAAAAGTTGGGGTGGATTAGTTACTCAACAAGGG
ATTAATGATTCAAGTGCTGATTTTGGTTTTGGAGTTTATAATGATCATCATTATCATTTA
GGATATTTTCTTTATGGAATTGGGGTTCTTGCAAAAATTGATCCTTTATGGGGACAAAAG
TATAAACCAATAGTTTATTCACTTTTGAAAGATTTTATGAACTTGGGCAAAAGAGATAAC
AAAAATTATCCAACTTTAAGGTGTTTTGATCCATACAAGTTACATTCTTGGGCTTCCGGG
GTGACTGAATTTGAAAATGGAAGGAATCAAGAAAGTTCGAGCGAAGCTGTGAATGCTTAT
TATTCGGCCGCATTAGTAGGTCTAGCTTACAATGACAAAAATCTTGTTGCTACCGGATCT
ACGCTTTTAGCATTGGAAATTAATGCCGTGCAAACTTGGTGGCATGTGAAAGCCGAAAGT
AATTTGTATGGTGAAGATTTTGCGAAAGAAAATAGGATTGTTGGTATTTTGTGGGCGAAT
AAAAGGGATAGTAAACTATGGTGGGCACCATCCGAGTGTCGAGAGTGTAGGCTTAGTATA
CAAGTTTTACCTTTGTTGCCTATTACCGAGACTTTGTTCAATGACGGTGTTTATGCTAAG
GAGCTTGTGGAGTGGACATTGCCATCTTTGAAGAATAAGACTAATGTTGAAGGATGGAAA
GGGTTTACCTATGCTTTGCAAGGTGTTTATGATAACAAAAATGCATTGAAGAAAATTAGG
TTGTTGAAAGGTTTTGATGATGGAAACTCTTTTAGTAATCTATTATGGTGGATTCATAGT
AGGTGA
TrGBP1 CDS1
>T. repens_CM019102.1
SEQ ID NO: 14
ATGTCTTCTGTTCCTTTCCTATTTCCTCAAACTCATTCAACTGTTCTTCCAAACCCTTCA
AATTTCTTCTCACAAAACTTACTATCCACACCCCTCCCTACTAACTCTTTCTTCCAAAAC
TTTGTTCTTAACAATGGTGACCAACCTGAATACATTCACCCATATCTCATCAAATCCTCA
AACTCTTCACTTTCTGTTTCATACCCTTTTCTCCTATTTTCAACAGCAATGTTGTACCAA
GTTTTCTCACCAGATCTCACCATTTCATCCTCACAAAAATCTCACTCAAACTCACCAAAA
AATAAGCATGTTATCTCATCCTATAGTGATCATGGTGTGACTCTTGATATTCCATCTTCT
AATCTAAGATTCTTTCTTGTTAGAGGAAGTCCTTTTGTAACAGCTTATGTTACAAAACCA
ACACCTCTTTCAATCACAACATTGCATAACATAGTTTCTTTGTCTTCTTTTGATAACAAA
AAAACCAAATTTACTCTTTTGCTCAACAATACTCAGAAATGGATCATATACACTTCTTCA
CCAATCAATTTAAACCATGATGGTTCCGAGGTTAAATCCGATCCATTTTCGGGGATTATT
CGTATCGCAGTTGTTCCTGATTCGAATTACGAGAAAATTCTCGATAAATTCAGCTCTTGT
TACCCTGTTTCTGGCTATGCAAACATTCAGAAGAAATTTGGTTTGGTTTATAAATGGCAA
ACAAAAAATTCAGGTGATTTACTTATGCTAGCACACCCTCTTCATGTTAAGCTTTTATCA
AAAAGTAACAATCATGGTGTTATTGTTTTGAATGATTTTAAGTATAGAAGTGTTGATGGT
GATCTTGTTGGTGTTGTTGGAAATTCATGGAATTTGAAAACTGATTCTATTGATGTAACA
TGGCATTCTAGTAAAGGTGTTACAAAAGAATCACATGATGAAATTGTTTCAGCACTTGTT
AAAGATGTGAAGGAATTGAATATTTCATCAATAGCAACAAATTCATCTTATTTTTATGGT
AAGATTGTTGGAAGAGCTGCAAGATTTGCATTGATAGCTGAAGAAGTTTCTTATTTTAAA
GTGATTCCAATTATTAAGAATTTTTTGAAGGAAACTATTGAGCCATGGTTAGATGGAAAT
TTCAAAGGGAATGGTTTTTTTTATGAAAAAAGTTGGGGTGGATTAGTTACTCAACAAGGG
ATTAATGATTCAAGTGCTGATTTTGGTTTTGGAGTTTATAATGATCATCATTATCATTTA
GGGTATTTTCTTTATGGAATTGGGGTTCTTGCAAAAATTGATCCTTTATGGGGACAAAAG
TATAAACCAAGAGTTTATTCAATTTTGAAAGATTTTATGAACTTGGGCCAAAGGGATAAC
AAAAATTATCCAACTTTAAGGTGTTTTGATCCATACAAATTGCATTCTTGGGCTTCCGGT
GCGACTGAATTTGAAAACGGAAGGAATCAAGAAAGTTCGAGTGAAGCTGTGAATGCATAC
TATTCGGCCGCATTAGTAGGTCTAGCATACAACGACAAAAATCTTGTTGCTACCGGATCT
ACGCTTTTAGCATTGGAAATTAATGCCGCGCAAACTTGGTGGCATGTGAAAGTTGAAAAT
AATTTGTATGGTGAAGATTTTGCGAAAGAAAATAGGATTGTTGGTATTTTGTGGGCGAAT
AAAAGGGACAGTAAATTATGGTGGGCACCATCCGAGTGTCGAGAGTGTAGGCTTAGTATA
CAAGTTTTACCTTTGTTGCCTATTACCGAGACTTTGTTCAATGATGGTGTTTATGCGAAG
GAGCTTGTGGAGTGGACATTGCCATCTTTGAAGAATAAGACTAATGTTGAAGGATGGAAA
GGGTTTACCTATGCTTTGCAAGGTGTTTATGATAAGAAAAATGCATTGAAGAAGATTAGG
ATGTTGAAAGGTTTTGATGATGGAAACTCTTTTAGTAATCTATTGTGGTGGATTCATAGT
AGGTGA
TrGBP1 CDS2
>T. repens_CM019114.1
SEQ ID NO: 15
ATGTCTTCTGTTCCTTTCCTATTTCCTCAAACTCATTCAACTGTTCTTCCAAACCCTTCA
AATTTCTTCTCACAAAACTTACTATCTACACCCCTCCCTACTAACTCTTTCTTCCAAAAC
TTTGTTCTTAACAATGGTGACCAACCTGAATACATTCACCCATATCTCATCAAATCCTCA
AACTCTTCACTTTCTGTTTCATACCCTTTTCTCCTATTTTCAACAGCAATGTTGTACCAA
GTTTTCTCACCAGATCTCACCATTTCATCCTCACAAAAATCTCACTCAAACTCATCAAAA
AATAAGCATGTTATCTCATCCTATAGTGATCTTGGTGTGACTCTTGATATTCCATCTTCA
AATCTAAGATTCTTTCTTATTAGAGGAAGTCCTTTTGTAACAGCTTTAGTTACAAAACCA
ACACCTCTTTCAATCACAACATTGCATACCATTGTTTCTTTGTCTTCTTTTGATAACAAA
AAAACCAAATTTACTCTTTTGCTCAACAATACTCAGAAATGGATCATATACACTTCTTCA
CCAATCAATTTAAACCATGATGGTTCCGAGGTTAAATCCGATCCATTTTCGGGGATTATT
CGTATCGCAGTTGTTCCTGATTCGAATTACGAGAAAATTCTCGATAAATTCAGCTCTTGT
TACCCTGTTTCTGGCTATGCAAACATTCAGAAGAAATTTGGTTTGGTTTATAAATGGCAG
ACAAAAAATTCAGGTGATTTACTTATGCTAGCACACCCTCTTCATGTTAAGCTTTTATCA
AAAAGTAACAATCATGGTGTTATTGTTTTGAATGATTTTAAGTATAGAAGTGTTGATGGT
GATCTTGTTGGTGTTGTTGGAAATTCATGGAATTTGAAAACTGATTCTATTGATGTAACA
TGGCATTCTAGTAAAGGTGTTACAAAAGAATCACATGATGAAATTGTTTCAGCACTTGTT
AAAGATGTGAAGGAATTGAATATTTCATCAATAGCAACAAATTCATCTTATTTTTATGGT
AAGATTGTTGGAAGAGCTGCAAGATTTGCATTGATAGCTGAAGAAGTTTCTTATTTTAAA
GTGATTCCAATTATTAAGAATTTTTTGAAGGAAACTATTGAGCCATGGTTAGATGGAAAT
TTCAAAGGGAATGGTTTTTTTTATGAAAAAAGTTGGGGTGGATTAGTTACTCAACAAGGG
ATTAATGATTCAAGTGCTGATTTTGGTTTTGGAGTTTATAATGATCATCATTATCATTTA
GGGTATTTTCTTTATGGAATTGGGGTTCTTGCAAAAATTGATCCTTTATGGGGACAAAAG
TATAAACCAAGAGTTTATTCAATTTTGAAAGATTTTATGAACTTGGGCCAAAGGGATAAC
AAAAATTATCCAACTTTAAGGTGTTTTGATCCATACAAGTTACATTCTTGGGCATCC
TsGBP1 CDS
>T. subterraneum_Tsud_chr4.g17370.1.am.mk
SEQ ID NO: 16
ATGTCTTCTGTTCCTTTCCTATTTCCTCAAACTCATTCAACTGTCCTTCCAAACCCTTCA
AATTTCTTCTCACAAAACTTACTATCCACACCCCTCCCTACTAACTCTTTCTTCCAAAAC
TTTGTTCTCAACAATGGTGACCAACCTGAATACATTCACCCTTATCTCATCAAATCCTCA
AACTCTTCACTTTCTGTTTCATACCCTTTTCTCCTATTTTCAACAGCAATGTTATACCAA
GTTTTTTCACCAGATCTCACCATTTCATCTTCACAAAAATCTCACTCAAACTCAACAAAA
AATAAGCATTTTATCTCATCCTATAGTGATCTTGGTGTAACTCTTGATATTCCATCTTCA
AATCTAAGATTCTTTCTTGTTAGAGGAAGTCCTTTTGTAACAGCTTCTGTTACAAAACCA
ACACCTCTTTCAATCACAACATTGCATAACATAGTTTCTTTGTCTTCTTTTGATAACAAA
AAAACCAAATATACTCTTTTGCTCAACAATACCCAGAAATGGATTATATACACTTCTTCA
CCAATCAATTTAAATCATGATGGTTCCGAGGTTAAGTCCGATCCATTTTCGGGGATAATT
CGTTTCGCGGTTGTTCCTAATTCGAATTACGAGAAGATTCTCGATAAATTCAGCTCTTGT
TACGCTGTTTCGGGATATGCAAATATTCAGAAGAAATTTGGTTTGGTTTATAAATGGCAA
AGGAAAAACTCAGGTGAATTACTTATGCTAGCACATCCTCTTCATGTTAAACTTTTATCA
AAAAGTAACAATCATGGTGTTACTGTTTTGAATGATTTTAAGTATAGAAGTGTTGATGGT
GATCTTGTTGGTATTGTTGGAAATTCATGGAATTTGAAAACTGATTCTATTGATGTAACA
TGGCATTCTAGTAAAGGTGTTACAAAAGAATCACATGATGAAATTGTTGCAGCACTTGTT
AAAGATGTGAAGGAATTGAATATTTCAGCAATAGAAACAAATTCATCTTATTTTTATGGT
AAGATTGTTGGAAGAGCTGCAAGATTTGCTTTGATAGCTGAAGAAGTTTCTTATTTTAAA
GTGATTCCAATTATTAAGAATTTTTTGAAGAAAACTATTGAGCCATGGTTAGATGGAAAT
TTTAAAGGAAATGGTTTTTTTTATGAAAAAAGTTGGGGTGGATTAGTTACTCAACAAGGG
ATTAATGATTCAAGTGCTGATTTTGGTTTTGGAGTTTATAATGATCATCATTATCATTTA
GGGTATTTTCTTTATGGAATTGGGGTTCTTGCAAAAATTGATCCTTTATGGGGACAAAAG
TATAAACCAATAGTTTATTCACTTTTGAAAGATTTTATGAACTTGGGCCAAAGGGATAAT
AAATTTTATCCAACTTTAAGGTGTTTTGATCCATATAAGTTGCATTCTTGGGCATCCGGG
GTGACTGAATTTGAAAACGGAAGGAATCAAGAAAGTTCGAGCGAAGCTGTGAATGCGTAC
TATTCGGCCGCATTAGTAGGTCTAGCGTACAATGACAAAAATCTTATTGCTACCGGATCT
ACGCTTTTAGCATTGGAAATTAATGCCGCGCAAACATGGTGGCATGTGAAAGTCGAAAAT
AATTTATATGGTGAAGATTTTGCGAAAGAAAATAGGATTGTTGGTATTTTGTGGGCGAAT
AAAAGGGACAGTAAACTATGGTGGGCAGCATCCGAGTGTCGAGAGTGTAGGCTTAGTATA
CAAGTTTTACCGTTGTTGCCTATTACGGAGACTTTGTTCAATGATGGTGTTTATGCTAAG
GAGCTTGTGGATTGGACATTGCCATCTTTGAAGAATAAGACTAATGTTGAAGGGTGGAAA
GGGTTTACCTATGCTTTGCAAGGTGTTTATGATAAGAAAAATGCATTGAAGAAGATTAGG
ATGTTGAAAGGTTTTGATGATGGAAACTCTTTTAGTAATCTATTGTGGTGGATTCATAGT
AGGTGA
LjGBP1 CDS
>L. japonicus_Lj1g3v3023590.1
SEQ ID NO: 17
ATGATTTTTATTACAAATAACGGTTCCAAAGGCAACACATATGCAAGAAGCTTCATTCTA
ACTTCTAAGGTCAACTTTCACCAATCCTCTCTAGTCTCTAATCTCACCAAAAACTCATAT
AAGAAGACAACACCACCACACAAACAATTGCACCAACATCTCATCTTCTCTCCACCAACA
ACAATGCCTCCTTCTTCTCCTTTCCTCTTCCCTCAAACCCAATCCACAGTCCTCCCAAAC
CCTTCAACCTTCTTCTCCCAAAACCTCCTCTCATCTCCACTCCCCACAAACTCCTTCTTC
CAAAACCTTGTCATCCAAAATGGTTCCCAACCTGAATACATTCACCCTTATCTCATCCAA
TCCTCAAACTCCTCCCTTTCTGCCTCATACCCACTTCTCTTCTTCTCTGCAGCACTCTTA
TACCAAACCTTTGTTCCAGATCTCACAATCTCTTCCACTATCAAAACCTCAAATCCTCAA
AACCATGTAATCTCATCTTACAGTGACCTTGGTGTCACATTAGACATCCCCAGTTCCAAT
TTGAGATTCTATCTAGCCAGAGGAAGCCCTTACATAACAGCCTCAGTGACCAAACCAACA
CCACTTTCAATCACAACAGTTCACTCCATAGTGTCTCTGTTATCCGCTGCTGACAAAACC
AAGCACACCCTTCAGCTCAATAACAATCAGACATGGCTAATATACTCTTCAGCCCCAATC
AATTTAAATAAACATGGAAGCTCTGAGCTTCAATCTGACCCATTTTCTGGGGTGATTCGT
ATAGCTGTTGTTCCTGATTCAACTTCAAACCCTAAGTATGAAGAAGTTCTTGACAAGTTC
AGTTCTTGCTACCCTGTTTCTGGGGATGCAAAACTCAAGGGGAATTTCACCGTGGTGTAT
AAATGGCAGAGGAAAAATTCAGGGGATTTGCTCATGCTAGCTCACCCTCTTCATCTCAAG
CTTCTCTCAAAAAACAAGCTTGCTGCCACTGTTCTCTATGATTTCAAGTATAGAAGCGTT
GATGGTGACCTTGTTGGTGTTGTTGGAGATTCATGGGTGTTGGAAGCTGAGCCTGTTCCT
GTAACATGGCATTCTAACAGAGGAATCAAAAAAGAATCTTATGGGGAAATTGTTTCAGCA
CTTTTGAAAGATGTGAAGGAGCTGAATTATTCTGCAGTGGCAACAAATTCTTCTTATTTT
TATGGGAAGCTTGTTGGAAGAGCTGCAAGGTTTGCATTGATAGCAGAAGAAGTTTCTTTC
CCAAAAGTGATTCCAAAGATTGTCAAGTTTCTGAAGGAGAGTATTGAGCCATGGTTGGAT
GGAACATTCAAAGGAAATGGCTTTCTCTATGAGACAAAATGGGGTGGGCTTGTTACTCAA
CAAGGGTCCAAAGATGCAGGTGCTGATTTTGGGTTTGGGATTTACAATGATCACCATTTC
CACTTGGGGTACTTTCTCTATGGAATTGCAGTTCTTGCAAAGATTGACCCTGCTTGGGGA
CAGAAATACAAGCCCCAAGCCTATGCACTTGTGAATGATTTCATGAACTTGGGACAAAGA
TACTACACTTTCTCTCCGCGGTTACGGTGTTTCGATCCTTACAAGATGCACTCTTGGGCC
TCGGGGTTAACCGAGTTCGAAAATGGGAGGAATCAGGAAAGTACAAGTGAAGCTGTGAAT
GCTTACTACTCAGCAGCATTGATGGGTCTAGCGTACGGCGACACACGTCTAGCTACCACT
GGATCAACACTCACGGCACTGGAAATTGGTGCTACACAAATGTGGTGGCATGTGAAAAAG
GAACAAATTTTGTACCCAGAAGAATTTGCAGAAGATAACAGAATTGTGGGGATTCTTTGG
GCTAACAAGAGAGACAGTAATCTATGGTGGGCTCCTGCTGAGTGCAGAGAATGCAGGTTA
AGTATCCAAGTTCTACCATTGTTGCCTGTTACTGAATCTTTGTTCTCTGATGCTGGTTAT
GCTAAGGAGCTTGTGGAGTGGACATTGCCTTCTTTGAAAAGCAAATCAAATGTAGAAGGG
TGGAAGGGGTTTACCTATTCCTTGCAAGGGATTTATGATAAGGAAATAGCATTGAAGAGT
ATAAGAATGTTGAAAGGTTTTGATGATGGGAACTCATACAGTAATCTGTTGTGGTGGATT
CATAGCAGATAA
LaGBP1 CDS1
>L. angustifolius_OIW16739
SEQ ID NO: 18
ATGTCTTCTCCTCCATTCCTCTTCTCCCAAACTCAATCCACAGTCCTTCCAAATCCATCA
ACTTTCTTCTCCCAAAACCTCCTCTCTTCTCCACTCCCTACAAACTCTTTCTTCCAAAAC
TTTGTTCTCAAAAATGGTGACCAACCTGAATACATTCACCCTTACCTCATCAAATCCTCA
AACTCTTCACTTTCTGTCTCTTACCCATTTCTCCTTTTCACCACAGCAATGCTTTACCAA
GTTTTTGTGCCAGATCTTACAATATCCACATCATCATCATCACATAAAAGTGAAACCAAA
ACTAGCCATGTAATTTCATCTTATAGTGATCTTGGTGTCACTTTGGATATTCCTTCTTCA
TATTTAAGATTCTTTTTAGTTAGAGGTAGTCCTTTTATAACAACTTCTGTTACAAAACCA
ACCACTCTTTCTATAACAACAACCAATAAAATTGTCTCATTGCATTCTTTTAATGACAAA
ACCAAACACACCCTTCAACTTCAAAACAACCAAACATGGCTTATATACACTTCTTACCCA
ATTGTCTTCTATCACAAAGACTATGCTATTGAATCAAACAAGTTTTCGGGTATTATTCGA
TTCGCGGCCTGGCCTGATTCCACCCCGAAATATGAGGAAATTCTTGACAAGTTTAGTTCT
TGTTACCCTGTATCAGGTGATGCAACAATTAAGAATCCGTTTCGGGTTGTTTATAAGTGG
CAAAGGAAAAGGAGTGGTGAATTGCTTATGTTAGCTCACCCTCTTCATGTTAAGCTTTTA
TCATCATCATTAGCATTTAACAATGTTACTGTTTTGAATGATTTTAAGTATAGAAGTGTA
GATGGTGATCTTGTTGGTGTTGTTGGTGATTCTTGGGTTTTGGAAACAGAACATGTTCCT
ATAACATGGCATTCTAAGAATGGTGTTAAGAAAGAATCATATAATGAGATTGTTTCAGCA
CTTTTTAAGGATGTTAAGGAGCTTAATGCTTCTAATGTAACAACAAATTCTTCTTACTTT
TATGGTAAGCTTGTTGGTCGAGCTGCGAGGCTCGCATTGATCGCGGAAGAGGTGTCTTAT
CTCGAAGTGATTCCGAAAATAAGTGATTTTTTGAAGGAGATGATTCAGCCTTGGTTGGAT
GGGAATTTCAAAGGGAATGGTTTTCTATATGAGAGAAAATGGGGTGGACTTGTTACTAAA
CAAGGGTCTATAGATGCAGGTGCTGATTTCGGGTTCGGAATTTACAATGATCATCATTTT
CATTTGGGGTATTTCCTTTATGGAATTGCAGTGCTTGCAAAGATTGATCCAGCATGGGGT
CAAAAATACAAGCCTCAAGCTTATGCACTTGTCACAGATTTTCTGAACTTAGGACAAAGA
TTCAACTCATATTCGCCGCGATTAAGGTGTTTCGATTTGTACAAGTTACACTCTTGGGCT
TCAGGGATAACCGAATTCGAAGACGGAAGGAATCAGGAGAGTACAAGTGAAGCTGTAAAT
GCATACTATGCAGCAGCATTACTCGGTCTAGCATATCGCGACACGCGACTCGTTGCGACT
GCATCGACTCTTACAGCATTGGAAATTCTAGCAGCACAAACTTGGTGGCATGTGAAATCC
GAAGACAAGTTGTATGATGAAGAGTTTACAAAAGATAACAGAATTGTGGGTATTTTGTGG
GCTAATAAGAGAGATAGTAAGCTATGGTGGGCTTCTTCGGAATGTAGAGAGTGTAGATTA
AGTATTCAAGTGTTGCCTTTGGTTCCTGTTACTGAATCATTGTTCTCTGATGCTGGTTAT
GTGAAGGAGCTTGTGGAATGGACTTTACCTTCTTTGAAGAATAAATCAAATGTTGATGGG
TGGAAAGGGTTTACCTATGCATTGCAAGGAATTTATGATAAAGAGAATTCATTGAAGAAG
ATTAGAATGTTGAAAGGTTTTGATGATGGAAACTCATTCAGTAATCTCTTATGGTGGATT
CATAGCAGATAA
LaGBP1 CDS2
>L. angustifolius_OIW17321
SEQ ID NO: 19
ATGGCTGCTCCTACTCCTTTCCTTTTCCCTGCAACTCAACCCACAATACTCCCTGACCCA
TCAACCTTTTTCTCTTCAAACCTTTCATCTCCACTTCCTACTAACTCTTTCTTCCAAAAC
TTTGTTCTTAATAGTGGGGAACAACCTGAATATATTCACCCTTATCTTGTCAAATCCACA
AAAAACTCACTTTCTATTGCATACCCTTTGCTCCTTTTCACTGCATCAGTGTTTTACCAA
ACTTTTGCGCCTGATCTCACTATATCTTCTGCTACACCCCAAGAATCTGCCGCAAAAAAC
CATGTTATCTCATCCTACAGTGACCTTGGTGTCACTTTGGACATTCCATCTTCAAATTTA
AGATTCTTTCTAGTTAGAGGAAGCCCTTATATAACTGCTTCTGTTACTAAACCAACCACT
CTTTCTATCAAAACAACTTCTCCTATAGAATCCTTAAATCCATCTAAGGACAACACCAAA
TACATTCTTAAACTGAAATCCGGTCAGACATGGATAATATATTCATCCTCCGCTATCAGT
TTAACCAAGGGGGAAACTGAAATCAGCTCAAACTCATTTTCTGGTATCATTCGATTCGCG
TCGTTGCGTAATCCTCAACAGGAGAGTACTCTTGACAAATACAGCTCCAGTTACCCGGTC
TCGGGTTATGCAGTGTTCAACAAATCGTTTAATGTGGTATATAATTTGGAAAAGGAAGGG
AATGGTGATTTACTCTTGCTAGCTCATCCTCTTCATGTTAAGCTTCTATCATCAAAATCT
AATAAAGTTACTGTTCTAAGTGACTTCAAGTATCCAAGTGTAGATGGTGAACTTGTTGGT
GTTGTTGGTGATTCATGGGAGTTAGAAACAAAACATGTTCCTTTAACATGGAATTCCGTA
AAAGGTGTGAAGAAAGAAGCATATGAAGAGATTGTTAAAGCGCTTGTTAATGATGTGAAT
GAGTTAAACTCATCAAATGTAACAACATCTTCATCTTACTTCTATGGAAAGCTTGTTGCT
AGGGCTGCAAGGCTTGCATTGATAGCAGAAGAGGTATCTAACAGTGAAGTGATTCCCAAA
ATCACTAAGTTTCTGAAGGATACGATTCAACCTTGGTTGGATGGTAGTTTCAAAGGGAAT
AGTTTTCTATATGAAAAAAAGTGGGGTGGACTTGTAACTAAACAAGGGTCTACAGATAAA
GGTGCTGATTTTGGTTTTGGGGTTTACAATGACCATCATTATCATTTGGGGTACTTCATT
TATGGAATTGCAGTGCTTGCAAAGATTGATACAGCATGGGGACAGAAGTACAAACCTCAA
GCTTATGCACTTGTGTCAGATTTTCTGAACACAGACCTAAAATCAAACTCACATTATCCA
CTTTTAAGGAACTTTGACGTGTACAAGTTACACTCTTGGGCTTCAGGGTTAACTGAATTT
GCAGATGGAAGGAATCAAGAAAGTACAAGTGAAGCTGTTAATGCTTACTATGCAGCAGCA
TTGATGGGTGTAGCATATCATGACATGGATCTAGTTCGCATTGCATCAACTGTGACAGCA
TTGGAAATTCATGCTGCACAAACATGGTGGCATGTGAAATCTGGAGACAAATTGTATGCA
GAAGAATTTGCTAAAGGGAACAAAATTGTGGGTATTGTATGGTCTAACAAGAGAGATAGT
AGTCTATGGTGGGCTTCAGCTGAAGCTAAAGAGTGTAGGTTAAGTATTCAAGTTTTGCCT
TTGTCTCCTATTACTGAAGCATTGTTCTCTGATGCTGCATATGTGAAGGAGCTTGTTGAA
TGGACTTTACCTTCTTTGAATAAACCAAATATAGAAGGGTGGAAAGGGTTTACCTATGCA
TTGCAAGGGATTTATGATAAAAGTAGTTCATTGGAGAAGATTAGAGCATTGAAAGGTGTT
GATGATGGGAATTCATTCACTAATCTCTTATGGTGGATTCATAGCAGATAA
LalbGBP1 CDS1
>L. albus_Lalb_Chr10g0092981
SEQ ID NO: 20
ATGCAGCAAAGCCTATATAAATCCAAAAAGTCCCCATTGCCATTCCATATGCATATCCTC
TCCTCAATTTCAATGGCTCACAACCTCCAACATGAACCTTTCCTCTTCCCACTAACCCAT
TCCACAGTCCTCCCTGACCCTTCTAACTTCTTCTCACCAAACCTTCTCTCAACTCCACTC
CCTACAAACTCTTTCTTCCAAAACTTTGCTCTCAAAAATGGTGACCAACCTGAATATATT
CACCCTTATCTCATCAAATCCTCAAACTCTTCACTTTCTGTCTCATACCCTTCTCACTTT
TTCACCACAGCTTTCATATACCAAGTTTTCATTGCTGATCTTACCATATCTGCTTCTGTT
AAAACCAACTCTGATTCTATACATAAGCATGTTATCTCTTCCTACAATGATCTTAGTGTT
ACATTGGATTTTCCTTCTTCAAATTTGAGGTTCTTTCTTGTTAGGGGAAGTCCTTTTCTT
ACAGCAAATGTTACTAGTAGTACACCACTTTCCATTACTACTATTCATGCTATACTTTCA
TTTTCTTCAAGTGATTCTCTTACCAAGTTTACTCTTAAGCTTAATAATAGCCAAACATGG
CTTATATATTCCTCTTCACCAATGAAATTCAGTCACACCCTCTCTGGTATTAGTTCTGAT
GCATTTTGTGGTGTGATTCGTATAGCAGTGTTGCCTGAGTCAAAAAATTCAAAATTTGAG
GAAATACTTGATAGGTTCAGTTCTTGTTACCCTATTTCTGGTGATGCTATACTCAAAAAA
CCATTTTCTGTTGTATATAAATGGGAAAAGAAAGGGTTAGGTGATTTGTTACTGTTAGCA
CATCCTCTGCATCTTCAAATGTTGTCTAAGAAAAATTCTGATGTTACTATTCTTGATGAG
TTTAAGTATAAAAGCATTGATGGGGACCTTGTTGGTGTTGTTGGTGATTCATGGTTATTG
AAAACAAAACCTGTTTATGTAACATGGCATTCAATACAAGGTGTAAAAAAAGAATCCTAT
AGTGAAATTGTTTCAGCACTTTCCAAAGATGTTGAAGGTCTAAATTCTGCTGCAATAACA
ACAGCTTCATCTTACTATTATGGGAAATTGGTTGCAAGGGCAGCAAGGTTGGCATTGATA
GCTGAAGAGATTGGATTTCGTGATGCGATTTCGGCGATCACCAAGTTCTTAAAGGAATCA
ATTGAGCCATGGCTTGATGGAACTTTAGAAGAAAATGGTTTTCTATATGATGAAAAATGG
GGTGGCCTTGTTACTAAACAAGGGTCTATTGATTCAGGTGCTGATTTTGGGTTTGGAATT
TACAATGATCATCATTACCATCTTGGGTATTTTCTATATGGAATTGCAGTGCTTGTGAAA
ATTGACCCATCATGGGGAATTAAGTATAAACCTCAAGCATATTCACTAATGCAAGATTTT
ATGAACCTAGGAGAAAAATCAAACTCAAATTACCCAACTTTAAGGTGTTTTGATCTATAT
AAATTGCATTCTTGGGCTGGTGGGTTAACTGAATTTGCAGATGGAAGAAATCAAGAGAGT
ACTAGTGAAGCTATAAATGCATATTATTCAGCAGCTTTATTAGGCCTAGCATATAATGAC
ACTAATATTTTTGAAACTGCATCAACTTTTGCATCATTAGAAATTCATGCAGCTAAGACA
TGGTGGCATGTGAAATTTGGTGATAATCTTTATGAGGAAGATTTTACAAAAGAGAATAGA
ATAATGGGTGTTTTATGGTCTAATAAAAGAGATAGTGGGTTATGGTTTGCACCTCCTGCA
ATGAAAGAGTGTAGGGTTGGAATTCAACTATTACCATTAGTACCTATTTCTGAAATGTTG
TTTTCTAATGTTAGTTTTGTGAAGGAACTTGTGAAGTGGACATTGCTAGCTTTGGATAGA
AATGATGTTGAAGATGAATGGAAAGGGTTTGTTTATGCATTGCAAGGAATTTATGATAAT
GAAAGTGCTTTGCAAAAGATTAGAAGATTGAAAGGTTTTGATGATGGGAACTCATTCACT
AATCTCTTATGGTGGATTCATAGCAGATGA
LalbGBP1 CDS2
>L. albus_Lalb_Chr04g0258421
SEQ ID NO: 21
ATGTCTGTTCCTACTCCTTTCCTCTTCCCTTCAATTCAATCGACAGTACTTCCTGACCCA
TCAAGCTTTTTCTCCCCAAACATTTCATCTCCACTTCCTACAAACTCTTTCTTCCAAAAT
CTTGTTCTAAATGGTGGAGGACAACCTGAGTATTTTCACCCTTATCTCATAAACTCCACA
AAAACCTCTCTTTCTGTTGCATACCCTTTGCTCCTTTTCACTGCATCAGTAGTGTACCAA
ACTTATGTGCCTGAACTCACCATATCTGCTACATCCCAAGAATCTGCCACAAAAAACCAT
GTTATCTCATCCTTCAGTGACCTTGGTGTCACTTTGGACCTACCCTCTTCAAATTTAAGA
TTCTTTTTAGTTAGAGGAAGCCCTTACATAACTGCTTCTGTTACTAAACCAACCACTCTT
TCTATCAACACATCTTCTGCTATTGAATCCTTAAGTGCATCATCTCATCGCAACACCAAA
TACATCCTTAAACTGAAATCCAAGCAGACATGGATAATATATTCATCCTCTCCTATCAGT
TTAACTAATGAGGGAACTGAAATCAGATCAAACTCATATTCTGGTATCATTCGATTCGCG
TCGTTGCGTAATCCTCACTATGAGAGCACTCTTGATAAATTCAGCTCCTCTTACCCGGTC
TCGGGTGATGCAGAGATCAAGAAACCGTTTCATTTGAGATATAAATGGCAGAAGAAAGGG
AATGGTGGTTTACTCATGCTAGCTCACCCTCTTCATGTTAAGCTTCTACCACGATTATTT
AGTCATGTCATTGTTCTACGCGATTTCAAGTATCCAAGTGTAGATGGTGATCTTGTTGGT
GTTGTTGGTGATTCATGGGAGTTAGAAACAAAACCTGTTCCTGTAACATGGCGTTCAGTA
AAAAGTGTGAAGAAAGAATCATATCAAGAGATTGTTAAAGCGCTTGTTAAAGATGTGAAC
GAGTTAAACTCATCAAATGTAACATCAACTTCATCTTACTTTTATGGAAAGCTTGTTGCT
AGGGCTGCAAGGCTTGCATTGATAGCAGACGAGGTGAATAATCATGAAGTGATTCCCAAA
ATCAGTATCTTTCTGAAGGAGACGATTCAGCCTTGGTTGGATGGAAGTTTCAAAGGGAAT
GCTTTTCTATATGAAAAAAGGTGGGGTGGACTTGTTACTAGACAAGGGTCTGTAGATAAA
GGTGCTGATTTTGGTTTTGGGGTTTATAATGATCATCATTATCATTTGGGGTACTTCCTT
TATGGAATTGCAGTGCTTGCAAAGATTGATACAGCATGGGCAAAGAAGTATAAATCTCAA
ACTTATGCACTTGTGACAGATTTTTTGAACACCGACCAAAGATTAAAACAATCTCCACGT
TTAAGGAATTTTGACTTATACATGTTACACTCTTGGGCTTCAGGGTTAACTGAATTTGGC
GATGGAAGGAATCAAGAAAGTACAAGTGAAGCTGTAAATGCTTACTATGCAGCAGCATTG
GTGGGTCTAGCATATGGCGACAAGCGTCTCATTAGCACTGCATCAACCCTAACAGCATTG
GAAATTCGTGCTGCACAAACATGGTGGCATGTGAAATCTAAAAACAAAGTGTATGCAGAA
GAATTTGCTAAAGGGAACAAAATTGTGGGTGTTCTTTGGTCTATCAAGAGAGACAGTGGT
CTATGGTGGGCTGCAGCTGAACGTAAAGAGTGTAGGCTAAGTATTCAAGTTTTGCCTTTG
TCACCTATTACTGAGTCATTGTTCTCTGATCCTTCATATGTGAAGGAGCTTGTGGAATGG
ACTTTACCTTCTGTGGAGAGTAAACAAAATGTTGAAGGGTGGAAAGGGTTTATCTATGCC
TTGCAAGGGATTTATGATAAAGGAAAATCATTGGAGAAGATAAGAACTTTGAAAGGTGTT
GATGATGGGAATTCATTCACTAATCTCTTATGGTGGATTCATAGCAGATGA
VuGBP1 CDS1
>V. unguiculata v1.1|Vigun05g034200.1 CDS
SEQ ID NO: 22
ATGTCTTCTTCATCTTCTTTTATGTTCCCTCAAACTCAATCCACAGTTCTCCCAGACCCT
TCAACCTACTTCTCCTCAAACCTTCTTTCATCTCCACTCCCCACAAACTCTTTCTTCCAA
AACTTTGTTCTTTCAAAAGGATCACAACCTGAGTATATTCACCCATACCTCATTCAAACC
TCAAAGTCCTCACTTTCTGCCTCATACCCTCTTCTCTTCTTCACTGCAGCAGTGTTGTAC
CAAACTTTTGTGCCGGATCTCACAATCTCTTCCAGTCAAACACTTCCAACTCAACAAAAC
CATGTAGTCTCATCATTCAGTGACCTTGGTGTCACTGTTGACATTCCCTCCTCCAACCTC
AGGTTCTTTCTCTCAAGAGGAAGCCCTTTTATAACTGCTTCTGTCACATCTTCAACATCT
CTTTCCATCACAACACTGCACACCATACTCTCTTTGTCTCCCAGTAATGACAAAAACACC
AAGTACACCCTTAAGCTCAACAACACTCAGACATGGCTCATATACGCCTCCTCCCCAATC
TATTTGAATCGTGATGGTGCTTCCCAGGTTACATCGAAACCATTCTCCGGCATCATTCGT
GTAGCAGCGTTGCCTGATGACAACCCCAACAATGTCGCAATTCTCGACAAGTTCAGCTCT
AGCTACCCTTCATCGGGTAATGCAACGCTACACGATCCTTTCCGTTTGGTGTATCAATGG
CAGAAGGAAGGTTCTGGGGATTTGCTCATGCTGGCTCACCCTCTTCATGCTAAACTTTTA
TCACATAATAACACCGGTAATGTCAATATTTTGCGTGATTTTAAGTATAGAAGCATTGAT
GGTGACCTTGTTGGTGTTGTTGGAGATTCATGGAAGTTGGAAATGAATCCTATTCCCGTG
ACATGGCATTCTAACAAAGGCGTGGGAAAAGAGTCATACAACGAAATTGTCTCAGCACTT
TCCAAGGATGTTCAAACCCTAAACTCTCCAATATCAACGCCATCCTCCTACGCAATTGGG
AAACTTATTGGAAGGGCTGCAAGGTTGGCGTTGATAGCGGAAGAAGTGTCTTTTCCAAAC
GTGGTTCCCACCATCAAGGAGTTTCTGAAGCGGAATATTCAGCCATGGTTGGATGGAACA
GTCCAAGGGAATGGCTTTCTATATGAAAAAAAATGGGGTGGACTTGTAACGAAAATGGGG
TCAACTGATTCAAGCGCTGATTTTGGGTTCGGAGTGTACAACGATCACCATTACCATTTG
GGGTACTTTCTTTACGGAATTGCGGTTCTAGCAAAGATAGACAACGAGTGGGGACAAAAA
TACAAGCCACAAGTTTATGCACTTTTGTCAGATTTCATGAACTTGGAGCAACAAAACGCT
CATTATCCACGTCTAAGGTGTTTTGACCTCTACAGGTTACACTCTTGGGCTTCAGGGGTG
ACAGAATTTGCAGATGGAAGGAACCAAGAAAGTACAAGTGAAGCTGTGAATGCATACTAT
TCAGCAGCTTTGGTGGGTGTAGCATACGGAGACAAAAGTCTTGTTAGCGCCGGATCAACG
CTATTGGCGATGGAAATTCTTGGTACACAAACATGGTGGCATGTGAAAGCAGAAGACAAG
TTGTACAATGAAGAGTTTGCAAAAAACAATAAGATAGTTGGTGTTCTGTGGTCTAACAAG
AGGGACAGTGGATTATGGTGGGCCCCTGCTACATGCAGAGAGTGCAGGCTTGGAATCCAA
GTGCTACCCTTGTCGCCGATCACTGAGACATTGTTCTCTGATGCTGGTTATGTGAAGGGG
CTTGTGGAATGGACATTGCCCTCTTTAAGTAGTGAGGCTTGGAAGGGAATGACCTATGCA
TTGCAGGGAGTTTATGATAAGCAAACAGCATTGCAGAACATAAGAAGGTTGAAAGGTTTT
GATGATGGGAACTCATTCACTAATCTCTTGTGGTGGATTCACAGCAGATAA
VuGBP1 CDS2
>V. unguiculata v1.1|Vigun05g034300.1 CDS
SEQ ID NO: 23
ATGTCTTCTTCATCTTCTTTTCTGTTCCCTCAAACTCAATCCACAGTTCTCCCAGACCCT
TCAACCTACTTCTCCTCCAACCTTCTTTCATCTCCACTCCCCACAAACTCTTTCTTCCAA
AACTATGTTATCCCAAACGGGTCACAACCTGAGTACATTCACCCTTACCTCATCACAACT
TCAAACTCCTCTCTATCAGCCTCATACCCTTTTCTCCTCTTCACCACAGCACTCTTGTAC
CAAGCTTTTGTGCCGGATCTCACCATCTCTTCCACTCAAACACAGTCACACGATCAACGA
AACCGTGTAATCTCATCATTCAGTGACCTTGGCATCACTTTGGACATTCCCTCCTCCAAC
CTCAGCTTCTTTCTCTCAAGAGGAAGCCCTTTTATAACTGCTTCCGTCACATCTTCAACA
TCTCTTTCTATCACAACACTGCACACCATACTCTCTTTGTCTCCCAGTAATGACAACAAC
ACCAAGTACACCCTTAAGCTTAACAACACCCAGACATGGCTCATATACGCCTCCGACCCA
ATCTATCTGAACCGTGACGGTGCTTCCGAGGTTACATCGAAGCCATTTTCTGGCATCATT
CGTGTAGCAGTGTTGCCTGATCCTAACTATGCGACAACTCTTGACAAGTTCAGCTCTTGT
TACCCTTTGTCGGGTGATGCAACACTGAAGGAGTCTTTCCGTTTGGTGTATCAATGGGAA
AAGGAAGGTTCTGGGGATTTGCTAATGCTGGCTCACCCTCTTCATGTTAAACTTTTATCA
AATAAAAGTAACGGGCAGGTTACTGTGCTGAGTGATTTTAAGTATAGAAGCATTGATGGC
GATCTTGTTGGTGTTGTTGGAGATTCATGGGTTCTGGAAACGGATCGTATTCCTGTGACA
TGGTATTCTAGCAAAGGAGTGGAAAAAGATTCGTACGATGAGATTGTGTCAGCGCTTGTT
AAGGATGTGGAGAAGCTTAACTCTTCCGCAATAGGAACAAGTTCATCTTATTTTTATGGA
AAGCGAGTTGGGAGAGCTGCAAGATTGGCACTGATAGCGGAAGAAGTGTCTTTTTCAAAG
GTTGTTCCCACCATTATGGATTTTCTTAAAGAAGCCATCGAGCCTTGGTTAGATGGAACT
TTCGTAGGGAATGGTTTTCTATATGAAAACAAATGGAGTGGACTTGTAACCAAACTAGGG
TCAACGGATTCAACCGCTGATTTCGGGTTTGGAGTTTACAATGATCACCATTATCATTTG
GGGTACTTTCTATATGGAATTGCGGTTCTTGCAAAGATTGATCCCGAGTGGGGACAAAAA
TACAAGCCACAAGTATATTCACTAGTGACAGATTTTATGAACTTGGGTCAAAGGTATAGC
AGAATTTATCCACGTCTAAGGTGTTTTGACCTTTACATGTTACATTCTTGGGCCGCAGGA
GTGACTGAATTTGAAGATGGTAGGAACCAAGAAAGTACAAGTGAAGCTGTGAATGCGTAC
TATTCAGCAGCGTTGGTGGGTCTGGCATATGGTGATTCAAATCTTGTTGAAACTGGGTCA
ACGTTAGTGGCGTTGGAAATTCTAGCTGCACAAACTTGGTGGCATGTTAAAGTGGAAGAC
AACTTGTACAATGAAGAATTTGCAAAAGACAATAGGATAGTGGGAATTTTGTGGGCTAAT
AAGAGGGATAGTAAGTTATGGTGGGCGAGTGCTGAATGTAGAGAATGCAGACTCGGAATC
CAAGTGCTACCCTTGTTGCCTATCACTGAGACATTGTTCTCTGATGCTGATTATGTGAAG
GAGCTTGTGGAATGGACATTGCCATCTTTAAGTAGTGAGGGGTGGAAGGGAATGACCTAC
GCATTGCAGGGAATTTATGATAAGGAAACAGCGTTGCAGAACATAAGAACGTTGACAGGT
TTTGATGATGGAAACTCTTACAGTAATCTCTTGTGGTGGATTCACAGCAGATGA
VuGBP1 CDS3
>V. unguiculata v1.1|Vigun05g034000.1 CDS
SEQ ID NO: 24
ATGTCTCCTTCTTTTCTATTCCCTCAAACTCAATCCACAGTTCTCCCAGACCCTTCAACC
TACTTCTCACCAAACCTTCTTTCTTCTCCATTCCCCACAAACTCTTTCTTCCAGAACTTC
GTTATTCCAAATGGTACACAGCCTGAGTATTTTCACCCCTATCACATTCAGGCCTCAAAC
TCCTCACTCTCTGCCTCCTACCCTTTTCTCTTCTTCACAGCAGCAGTGTTGTACCAAGTT
TTTGTCCCAGATCTCACCATTTCAGCCTCTCAAACAACCTCCTATGGACAAAACCGTGTT
ATCTCATCCTACAGTGACCTCGGTGTCACTTTGGACATCCCAAGTTCCAACCTCAGGTTC
TTTCTTGTCAGAGGAAGCCCTTTCATAACTGCTTCTGTCACAAAACCAACCTCTCTTTCC
ATCAAAACAGTGCACACCATACTCTCTTTGTCTTCCTATGATGGCAATACCAAGTTTATC
ATTCAGCTTAACAACACTCAGACATGGCTCATATACACCTCGTCCCCTATCTATTTGAAC
CATGTTCCTTCCGAGGTTACATCCAAGCCGTTTTCTGGCATCATTCGTATAGCAGCGTTG
CCTGATTCCAACCCCAGTAATGTCGCAACTCTTGACAAGTTCAGTTCTTGTTACCCCGTG
TCGGGTGATGCAACACTCGGCAAGCCTTTCCGTTTGGAGTATAAATGGCAAAAGAAAAGG
TCAGGGGACTTGCTCATGCTAGCTCACCCTCTTCATGCTAAGCTTCTATCACGTGACTGT
AACGTTACCGTTCTGCACGATTTTAAGTATCGAAGTGTTGACGGTGATCTTGTTGGTGTT
GTTGGAGATTCGTGGGTGTTGGAGACGGATCCTATTCCTGTCACATGGCATTCTAAGAAA
GGGATCAGTAAAGAGTCGTTTGGTGAGATTGTTTCTGCACTTTATAAGGATGTCAAGGGG
CTGAATTCTTCTGCAATAACAACAAATTCATCTTATTTCTATGGGAAGCTTGTTGGAAGG
GCTGCAAGGTTAGCCTTGATCGCAGAAGAAGTGTCTTATTACAAGGTGATTCCCAAGATT
AGAAAGTTTCTGAAGGAAACCATTGAGCCCTGGTTGGATGGAACTTTCAAAGGGAATGGT
TTTCTATACGAAAGAAAATGGCGTGGACTTGTTACTGAACAAGGCTCCACAGATTCAACT
GCTGATTTTGGTTTTGGAATATATAACGATCACCATTTTCATTTGGGGTACTTCCTTTAT
GGAATTGCAGTTCTTGCAAAGATTGACCCTGCCTGGGGCAAAAAATTCAAACCGCAAGCT
TATTCACTTGCGACAGATTTTATGAACTTGGGCCAAAGATATAACTCAGATTATCCACGC
CTAAGGTGTTTTGACCTTTACAAGTTACACTCTTGGGCTTCAGGGCTGACTGAATTTGAA
GATGGAAGGAATCAGGAGAGTACAAGCGAAGCTGTAAATGCATACTATGCAGCAGCTTTG
ATGGGTCTGGCTTATGGTGATAGCCGTCTTGTTGATACTGGATCGACACTGTTAGCATTG
GAAATTCGTGCTACACAAACATGGTGGCATGTAAAAGCAGAAGACAACTTGTATGAAGAA
GAATTTGCAAAAGATAACAGGATCGTGGGTATTCTGTGGGCTAACAAGAGGGACAGTAAG
CTATGGTGGGCTACTGCGGAATGTAGAGAGTGCAGGCTTAGTATCCAAGTTCTACCCTTG
TTACCTGTCACAGAGACCTTGTTCTCTGATACTGTTTATACGAAGGAGCTTGTGGAATGG
ACACTACCTTCTTTGAAGAATAAAACGAATGTAGAAGGCTGGAAGGGATTCACCTATGCC
TTGCAAGGAATTTATGATAAAAGTACAGCATTAAAGCAAATAAGAAGGTTGACAGGTTTT
GATGATGGAAACTCATTCAGTAACCTCCTCTGGTGGATTCACAGCAGATGA
PvGBP1 CDS1
>P. vulgaris v2.1|Phvul.008G033200.1 CDS
SEQ ID NO: 25
ATGTCTTCTTCATCTTCTTTTCTCTTCCCTCAAACTCAATCCACAGTTCTTCCAGACCCT
TCAACCTACTTCTCCTCTAACCTTCTTTCATCTCCACTTCCCACAAACTCTTTCTTCCAA
AACTATGTTATCCCAAACGGGTCACAACCTGAGTACATTCACCCTTACCTCATCAAAAGT
ACAAACTCCTCACTATCAGCCTCATACCCTCTTCTCCTCTTCACCACAGCACTCTTGTAC
CAAGCTTTTGTGCCAGATCTCACCATCTCTTCCACTCAAACACACTCACAGCAACAAAAC
CGTGTAATCTCATCATTCAGTGACCTTGGTGTCACTTTGGATATTCCCTCCTCCAACCTC
AGGTTCTTTCTCTCAAGAGGAAGCCCTTTTATAACTGCTTCCGTCACTTCTTCAACATCT
CTTTCTATCACAACACTGTACACCATACTCTCTTTGTCTTCCAACAATGAGAACAACACC
AAGTACACCCTTAAGCTTAACAACACTCAGACATGGCTCATATACACCTCCTCCCCCATC
CATTTCAACCATAATGCTTCAGAGGCTACGTCCAAGCCATTTTCTGGCATCATTCGTGTA
GCAGTGCTGCCAAATCCTAACTATGAGACGATTCTTGACAAGTACAGCTCTTGTTACCCT
TTGTTGGGTGATGCAACACTAGAGGAGCCTTCCCGTGTGGTGTATCAATGGCAAAAGGAA
GGGTCTGGGGATTTGCTCATGCTGGCTCACCCTCTTCATGTTAAGCTTTTATCAAATAAT
AATAACGGGAATGTTACTTTGCTGAGTGATTTTAAGTACAGAAGCATTGATGGTGATCTT
GTTGGTGTTGTTGGAGATTCATGGATATTGCAAACGGATCGTATTCCTGTGACATGGTAT
TCTAACAACGGAGTGGAAACAAATTCATATGATGAGATTGTCTCAGCGCTTGTTAAGGAC
GTGCAAGCGCTTAATTCTTCAGCAATAGGAACAACTTCATCTTATTTTTATGGAAAGCGC
GTTGGAAGGGCCGCAAGGTTGGCATTGATAGCGGAAGAAGTGTCGTTTTCAAAGGTTGTT
CCCACGGTTACGGATTTTCTTAAAGAGGCCATTGAGCCTTGGTTAGATGGAACTTTCGAA
GGGAACGGTTTTCTATATGAAAATAAATGGGGTGGACTTGTAACCAAACTGGGGTCAACG
GATTCAAGCGCTGATTTTGGGTTTGGAGTTTACAATGATCACCATTACCATTTGGGGTAC
TTTCTATATGGAATTGCGGTTCTTGCAAAGATTGATCCCGAGTGGGGACAAAAATACAAG
CCACAAGTTTATTCACTTGTGACAGATTTTATGAACTTGGGTCAAAGGTATAACAGAAAT
TATCCACGTCTAAGGTGTTTTGACCTTTATACGTTACATTCTTGGGCTGCGGGAGTGACT
GAATTTGAAGATGGTAGGAATCAAGAAAGCACGAGTGAAGCTGTGAATGCATACTATTCA
GCAGCGTTGGTGGGTCTGGCATATGGTGACTCGAGTCTTGTTGCCACTGGGTCAACGTTG
GTGGCGTTGGAAATTCTAGCTGCACAAACTTGGTGGCATGTGAAAGTGGAAGACAACTTG
TACGAAGAAGAATTTGCAAAAGACAATAGAATAGTGGGGATTGTGTGGGCTAATAAGAGG
GATAGTAAGTTATGGTGGGCCGGTGCAGACTGTAGAGAATGCAGACTTGGAATCCAAGTG
CTACCCTTGTTGCCTATCACTGAGACACTGTTCTCTGATGCTGATTATGTGAAGGAGCTT
GTGGAATGGACATTTCCCTCTTTAAGTAGTGAGGGGTGGAAGGGAATGACCTATGCCTTG
CAAGGAGTTTATGATAAGCAAACAGCACTGCAGAATATAAGAACATTGAAAGGTTTTGAT
GATGGAAACTCTTACAGTAATCTCTTGTGGTGGATTCACAGCAGATAA
PvGBP1 CDS2
>P. vulgaris v2.1|Phvul.008G033100.1 CDS
SEQ ID NO: 26
ATGTCTTTCTCATCTTCTTTTCTCTTCCCTAAAACTCAATCCACAGTTCTCCCAGACCCT
TCAACCTACTTCTCTTCAAACCTTGTTTCTTCTCCTCTCCCCACAAACTCTTTTTTCCAA
AACTTTGTCCTTTTAAACGGGTCACAACCTGAGTACATTCACCCCTACCTCATCCAAACC
TCAAAGTCCTCACTCTCTGCCTCATACCCTCTTCTCTTCTTCACTGCAGCAGTGTTGTAC
CAAACTTTTGTGCCGGATCTCACAATCTCTTCCACTCAAACACTTCCAAATGAACAGAAC
CATGTAATCTCATCCCACAGTGACCTTGGTGTCACTTTGGACATTCCCTCCTCCAACCTC
AGGTTCTTTCTCTCAAGAGGATGCCCTTTTATAACTGCTTCCGTCACATCTTCAACATCT
CTTTCTATCAGAACACTGCACACCATACTCTCTTTGTCTTCCAACAATGAGAACAACACC
AAGTACACCCTTAAGCTTAACAACACTCAGACATGGCTCATATACACCTCCTCCCCCATC
CATTTCAACCATAATGCTTTAGAGGTTACGTCCAAGCCATTTTCTGGCATCATTCGTGTA
GCAGTGCTGCCAAATCCTAACTATGAGACAATTCTTGACAAGTACAGCTCTTGTTACCCT
TTGTTGGGTGATGCAACACTAGAGGAGCCTTCCCGTGTGGTGTATCAGTGGCAAAAGGAA
GGGTCTGGGGATTTGCTCATGCTGGCTCACCCTCTTCACGTTAAGCTTTTATCAAATAAT
AATACTGGTACTGACACTATTTTGCATAATTTTAAGTATAGTAGCATTGATGGTGATCTT
GTTGGCGTTGTTGGAGATTCATGGAAGTTGGAAATGAATCATATTCCTGTAACATGGCAT
TCTAACAAAGGAGTGGAAAAAGAGTCATATGATGAAATTGTCTCAGCACTTTCCAAGGAC
GTTCAAGCACTAAACTCTTCACCAATAGCAACAGCATCCTCCTATTTATATGGGAAACTT
ATTGGAAGGGCTGCAAGGTTGGCGTTGATAGCGGAAGAAGTGTCTTTTCCAAACGTGGTT
CCAACGATTAAGGAGTTTCTGAAGAAGAATATTGAGCCTTGGTTGGATGGAACATTCCAA
GGGAACGGTTTTCTATATGAAAATAAATGGGGTGGACTTGTAACAAAACTGGGGTCAACA
GATTCAAGCGCTGATTTTGGGTTTGGAGTGTACAATGATCACCATTACCATTTGGGTTAC
TTTCTTTATGGAATTGCGGTTCTAGCAAAGATTGACCCTGAGTGGGGACAAAAATACAAG
CCACAAGTTTATTCACTTTTGTCAGATTTTATGAATTTGGACCACCAACACAACGCTTAT
TATCCACGTCTAAGGTGTTTTGACCTCTACATGTTACATTCTTGGGCTTCAGGGTTGAAA
GAATTTGCAGATGGACGGAACCAAGAAAGTACAAGTGAAGCTGTGAATGCGTACTATTCA
GCAGCTTTGGTGGGTCTAGCATATGGTGACTCAAGTCTTGTTGCCACTGGGTCAACGTTA
GTGGCGTTGGAAATTCTTGCTGCACAAACTTGGTGGCATGTGAAAGTGGGAGAGAAGTTG
TACAAAGAAGAGTTTGCAAAAGACAATAGGATAGTTGGTGTTCTGTGGGCTAATAAGAGA
GATAGTGGACTATGGTGGGCGAGTGCAGAGTGTAGAGAATGCAGACTTGGAATCCAAGTG
CTACCCTTGTTGCCTATCACTGAGACATTGTTCTCTGATGCTGATTATGTGAAGGAGCTT
GTGGAATGGACACTTCCCTCTTTAAGTAGTGAGGGGTGGAAGGGAATGACCTATGCCTTG
CAAGGAGTTTATGATAAGCAAACAGCATTGCAGAATATAAGAACGTTGAAAGGTTTTGAT
GATGGAAACTCTTACACTAATCTCTTGTGGTGGATTCACAGCAGATAA
PvGBP1 CDS3
>P. vulgaris v2.1|Phvul.008G033000.1 CDS
SEQ ID NO: 27
ATGTCACAATTCCCCTTCTCTTCCTCACAAACAATGTCTTCTTCTTTTCTCTTCCCTCAA
ACTCAATCCACAGTCCTCCCAGACCCTTCAACCTACTTCTCACCAAACCTCCTTTCATCT
CCACTCCCCACAAACTCTTTCTTCCAAAACTTTGTTATTCCAAATGGCACAGTGCCTGAG
TACTTTCATCCCTACCACATTCAGTCCTCAAACTCCTCACTCTCTGCCTCCTACCCTTTT
CTCTTCTTTACAGCAGCTGTGTTGTACCAAGTTTTTGTCCCAGATCTCACCATCTCTGCC
TCTCAGACATACTCAAATGCACAAAACCGTGTAATCTCTTCCTACAGTGACCTTGGTGTC
ACTTTGGACATTCCCACTTCCAACCTCAGGTTCTTTCTTGTCAGAGGAAGCCCTTTCATA
ACTGCTTCTGTCACAAAGCCAACCTCTCTTTCCATCACAACCGTGCACACCATACTTTCT
TTGTCTTCCTATGATGACAACACCAAGTTTATCCTTCAGCTTAACAACACTCAGACATGG
CTCATATACACCTCCTCCCCAATCTATTTGAACCATGCTGCTTCTCAGGTTTCATCCAAG
CCATTTTCTGGCATCATCCGTATAGCAGCTTTGCCTGATTCCAACCCCAACAATGTCGCA
ACTCTTGACAAGTTCAGTTCTTGTTACCCTGTGTCGGGTGATGCAGCACTCAAGAAACCT
TTCCGTGTGGAGTATAAATGGCAAAGGAAAAGGTCAGGGGACTTGCTCATGCTAGCTCAC
CCTCTTCATGCTAAGCTTCTATCACACGATTGTAACGTTACCGTTCTGCACGATTTTAAG
TATAGAAGTGTTGACGGTGATCTTGTTGGTGTTGTTGGAGATTCATGGGTGTTGGAAACG
GATCCTATTCCTGTCACATGGCATTCTAAAAAAGGCATCGATAAAGAGTCATTTGGTGAG
ATTATCTCAGCGCTTAATAAGGATGTGAAGGAGCTAAATTCTTCTGCAATAACAACACAG
TCATCTTATTTCTATGGGAAGCTTGTTGGAAGGGCTGCAAGGTTGGCCTTGATCGCAGAA
GAAGTGTCTTATCCTAAAGTGATTCCCAAGATTAGAAATTTTTTGAAGGAAACCATTGAG
CCCTGGTTGGATGGAACTTTCAAAGGGAATGCTTTTCTATATGAAAGAAAATGGCGTGGA
CTTGTTACTAAACAAGGCTCCACGGATTCAACTGCTGATTTTGGGTTTGGAGTGTATAAC
GATCACCATTTTCATTTGGGGTACTTTATTTATGGAATCGCAGTTCTTGCAAAGATTGAC
CCTGCCTGGGGCAAACAATACAAACCGCAAGCCTATTCACTTGTGACAGATTTTATGAAC
TTGGGCCAAAGATATAACACAGATTATCCGCGCCTAAGGTGTTTTGACCTTTACAAGTTA
CACTCTTGGGCTTCAGGGCTGACTGAATTTGAAGATGGAAGGAATCAGGAGAGTACAAGT
GAAGCTGTAAATGCCTACTATGCAGCAGCATTGATGGGTCTAGCTTATGGTGATAGCCGT
CTTGTTGATACTGGATCAACACTGTTAGCATTGGAAATTCGTGCTACACAAACATGGTGG
CATGTAAAAGTGGAAGACAACTTGTATGAAGAAGAATTTGCAAAAGATAACAGGATAGTG
GGTATTCTGTGGGCTAACAAGAGGGACAGTAAGCTATGGTGGGCTCCTGCAGAGTGCAGA
GAGTGTAGGCTTAGTATCCAAGTTCTACCCTTGTTGCCTGTCACTGAGACCTTGTTCTTT
GATACTGTTTATGCCAAGGAGCTTGTGGAATGGACACTGCCTTCTTTGAAGAACAAAACA
AATGTAGAAGGCTGGAAGGGATTCACCTATGCCTTGCAAGGAATTTATGATAAAACTACA
GCATTAAAGAAAATAAGAATGTTGACAGGTTTTGATGATGGAAACTCATTCAGTAATCTC
CTGTGGTGGATTCACAGCAGATAA
GmGBP1 CDS1
>G. max Wm82.a2.v1|Glyma.08G245600.1 CDS
SEQ ID NO: 28
ATGTCTTCTTTTCTTTTCCCTCAAACACAATCCACAGTCCTCTCAGACCCTTCAACCTAT
TTCTCCTCAAACCTCCTTTCATCTCCACTCCCCACAAACTCTTTCTTCCAAAACTTCGTT
ATTCCAAACGGGTCCCAAGCTGAGTACATTCACCCTTACCTCATCAAAACCTCAAACTCT
TCACTATCAGCTTCATACCCTCTTCTGATCCTCTTCACCACTGCAGTGTTGTACCAGACT
TTTGTGGCAGATATCACCATCTCTTCAACTCAAACAACCTCACAAAACCATGTAATCTCA
TCATACAGTGACCTTGGTGTCACTTTGGACATTCCCTCCTCCAACCTAAGGTTCTTTCTC
TCAAGGGGAAGCCCTTTTCTAACCGTTTCTGTGACATCTCCAACATCTCTTTCCATCACA
ACAGTGCATACCATAGTCTCTTTGTCTTCCAATGATGACAACAACACCAAATACACCCTT
AAGCTTAACAACACTCAAACATGGCTCATATACACTTCCTCACCAATCTATTTCACCCAT
AATAATGCTTCAGAGGTTACATCCAAGCCATTTTCTGGCATCATTCGTGTGGCAGTGTTG
CCTAACCACAACTACGTAACAATTCTTGACAAGTTCAGCACTTGTTACCCTTTGTCGGGT
AATGCAACACTCGTAGAGCCTTTCCGTGTGGTGTATGAATGGCAAAAGGAAGGTTCTGGG
GACTTGCTCATGCTAGCTCACCCTCTTCATGTTAAGCTTCTATCAAATAATTATAATGGT
CTAGTTACTGTGCTGAACGATTTTAAGTATAGAAGCATTGATGGTGATCTTGTTGGTGTT
GTTGGAGACTCATGGGTGTTGGAAACCAATCCTATTCCTGTGACATGGTATTCCAACAAA
GGTATGGAAAAAGATTCTTATGATGAGATTGTCTCGGCACTTGTTAAGGATGTGCAAGAG
CTGAATTCTTCATCAATAGGAACAAGTTCATCTTATTTTTATGGAAAGCGTGTTGGAAGG
GCTGCAAGGTTGGCGTTGATAGCGGAAGAAGTTTCTTTTTCTAACGTGGTTCCCACGATT
AAGAAGTTTCTTAAGGAGTCTATTGAGCCTTGGTTGGATGGAACTTTACAAGGGAATGGA
TTTCTATACGAAAATAAATGGGGTGGACTTGTCACCAAACTGGGGTCAACGGATTCAACA
GCTGATTTTGGGTTTGGAGTGTACAATGATCACCATTATCATTTGGGATACTTCCTTTAT
GGAATTGCGGTCCTTGCAAAGATTGATCCTGAGTGGGGACAAAAATACAATCCACAAGTT
TATTCACTTGTCACAGATTTTATGAACTTGGGCCAAAAATATAACTCTCGTTATCCACGT
CTAAGGTGTTTTGACCTTTACAACTTACACTCTTGGGCTTCAGGAGTGACTGAATTCGCA
GATGGAAGGAATCAAGAAAGTACAAGTGAGGCTGTGAATGCGTACTATTCAGCGGCATTG
GTAGGTTTAGCATATGGTGACTCAAATCTTGTTGCCATTGGATCAACACTACTGGCTTTG
GAAATTCTTGCTGCACAAACTTGGTGGCACGTGAAAGCAGAAGGCAACTTGTACGAAGAA
GAATTTGCAAAAGAGAACAAAATAGTGGGTGTTCTGTGGGCTAACAAGAGAGATAGTGCC
CTATGGTGGGGCCCTGCTACGTGTAGAGAGTGTAGGCTTGGAATTCAAGTGCTACCATTG
TCTCCTGTTACTGAGACTTTGTTCTCTGATGCTGATTATGTGAAGGAGCTTGTGGAATGG
ACAATGCCCTCTTTGACTAGTGAAGGGTGGAAGGGAATGACCTATGCCTTGCAAGGAATT
TATGATAAGGAAACAGCATTGGAAAATATTAGAAAGTTGAAAGGTTTTGATGATGGGAAC
TCGTTGAGTAATCTCTTGTGGTGGATTCACAGCAGATGA
GmGBP1 CDS2
>G. max Wm82.a2.v1|Glyma.08G246000.1 CDS
SEQ ID NO: 29
CTCCCAAACCCTTCAACCTACTTCTCCTCAAACCTTGTTTCATCTCCACTCCCCACAAAC
TCTTTCTTCCAAAACTTTGCTCTTCAAAATGGGACACAAGCTGAGTACATTCACCCTTAC
CTCATCAAAACCTCAAACTTTTCACTCTCAGCCTCATGCCCTCTTCTCCTCTTCACCACA
GCAGTGTTGTACCAGACTTTTGTGGCAGATATCACTATCTCTTCAACTCAAACAACCTCA
CAAAACCATGTAATCTCATCATACAGTGACCTTGGCGTCACTTTGGACATTCCCTCTTCC
AACCTAAGAGGAAGCCCTTATATAACCGCTTCCGTAACAAAGCCAACATCTCTCTCCATC
ACAACAGTGCGCTCCATAGTTTCTTTGTGTTCCAATAATAAGGAAAACACCAAGTACACC
CTTAAGCTTAACAACACTCAGACATGGCTCATATACACCTCCTCCCCAATATATTTGAAC
CATGATGCTGCTTCCAACATTACTTCCAAGCATTTTCTGCAGTGTTGCCTGTTTCCAACT
TCAAGAGTGTGGCAATTCTCGACAAGTTCAGCTCTTGTTACCCGGTCTTTATCGGGTAAT
GCAACACTCGTGAAGCCTTTTCGTGTGACGTATGAGTGGCAAAAGAAAGGGCCTGGGTTC
TTGCTCACGCTAGCTCACCCTCTTCATGTCAAGCTTCTACAATATAAAAAGAATCATCGC
ATGATTGTTCTGCGTGATTTTAAGTATAGAAGCATTGATGGTGATCTTGTTGGTGTTGTT
GGAGATTCATGGCTGTTAAAAACTGATACCATTCCTGTGACATGGCATTCTAACAAAGGT
GTGGAAAAAGAGTCACATGATGAGATTGTCTCAGCGCTTTCTAAGGATGTTGAAGCGCTA
AGTTCTTCACCAATAGCAACAGAATCGTCTTATTATTATGGGAAACTTATTGGAAGGGCT
GCAAGGTTGGCGTTGATAGCAGAAGAAGTGTCTTCTCCTAATGTGATTCCAACGATTCAG
AAGTTTCTGAAGGATAGTATTGAGCCTTGGTTGGATGGAACTTTCCAAGGGAATGGTTTT
CTATATGAAAACAAATGGGGTGGACTTGTCACCAAACAAGGGTCAACAGATTCAGGAGCT
GATTTTGGGTTTGGAGTGTACAATGATCACCATTATCATTTGGGGTACTTTCTTTATGGA
ATTGCGGTTCTTGCAAAGGTTGACCTTCAATGGGGACAAAAGTACAAGCCACAAGTTTAT
TCACTTGTGTCAGATTTTATGAACTCGGGCCAAAAATATAACTCACATTATCCACGTCTA
AGGTGTTTTGACCTTTACAAGTTACACTCTTGGACTTCAGGGGTGACTGAATTTACAGAT
GGACGGAATCAAGAAAGTACAAGTGAGGCTGTGAATGCGTACTATTCAGCAGCATTGGTA
GGTTTAGCATATGATGACTCAAATCTTGTTGCCACTGGGTCAACACTACTAGCTTTGGAA
ATTCTTGCTGCACAAACTTGGTGGCATGTGAAAGCAGAAGGCAACTTGTACGAAGAAGAA
TTTGCAAAAGAGAACAAAATAGTGGATGCTCTGTGGGCTAACAAGAGAGATAGTGCACTA
TGGTGGGCCCCTGCTACGTGTAGAGAGTGTAGGCTTGGAATCGAAGTGCTACCATTGTCT
CCTGTTACTGAGACATTGTTCTATGATGCTGATTATGTGAAGGAGCTTGTGGAATGGACA
ATGCCCTCTTTGACTAGTGAAGGATGGAAGGGAATGACATATGCCTTGCAAGGAATTTAT
GATAAGGAAACAGTTTTGCAGAATATTAGAATGTTGACAGGTTTTGATGATGGGAATTCA
TTCACTAATCTCTTGTGGTGGATTCACAGCAGATGA
GmGBP1 CDS3
>G. max Wm82.a2.v1|Glyma.18G266900.1 CDS
SEQ ID NO: 30
ATGTCTTCTAATTTTCTCTTCCCTCAAACTCAATCCACAGTCCTCCCAAACCCTTCAACC
TACTTCTCCTCAAACCTTCTTTCTTCTCCACTCCCCACTAACTCTTTCTTTCAAAACTTT
GTTATTCCAAACGGGTCCCAAGCTGAGTACATTCACCCTTACCTCGTCAAAACCTCAAAC
TCTTCACTCTCAGCCTCATACCCTCTTCTCCTCTTCACCACAGCACTTTTGTACCAATCT
TTTGTGCCAGATATCACAATCTCTTCCACTCAAACACACTCAAATCAACAAAACCGTGAA
ATCTCATCATACAGTGACCTCAGTGTCACTTTGGACATTCCCTCCTCCAACCTAAGGTTC
TTTCTCTCAAGAGGAAGCCCTTTTATAACCGCTTCTGTGACATCTCCAACATCTCTTTCG
ATCACAACAGTCCACACCATAGTCTCTTTGTCTTCCAATGACGACAACAACACCAAGTAC
ACCCTTAAGCTTAACAACACTCAAATATGGCTCATATACACCTCCTCCCCAATCTATTTG
AATCATGATGGCGCTTCCAATATTACATCCAAGCCATTTTCTGGCATAATTCGTGTAGCA
GCGCTGCCTGATTCCAACTCCAAGAGTGTAGCAATTCTCGACAAGTTCAGCTCTTGTTAC
CCTTTGTCGGGAAATGCAACACTCGTGGAACCTTTCCGTGTGGTGTATCAATGGCAAAAG
GAAAGTTCTGGGGACTTGCTCATGCTAGCTCACCCTCTTCATGTTAAGCTTTTATCAAAT
AGTCAAGTTACTGTGCTGAAAGATTTTAAGTATAGAAGCATTGATGGCGATCTTGTTGGT
GTTGTTGGAGATTCATGGGTGTTGGAAACGGATCCTATTCCTGTGACATGGTATTCTAAC
AAAGGTGTGGATAAAGATTCGTATGATGAGGTTGTCTCGGCACTTGTTAAGGATGTGCAA
GAGCTAAATTCTTCAGCAATAGGAACCAGTTCATCATATTTTTATGGGAAGCGTGTTGGA
AGGGCTGCAAGGTTGGCGTTGATAGCGGAAGAAGTGTCTTTTTCCAACGTGGTTCCCACG
ATTAAGAAGTTTCTGAAGGAGTCTATTGAGCCTTGGTTGGATGGAACTTTTCAAGGGAAT
AGTTTTCTATATGAAAATAAATGGGGTGGACTTGTCACCAAACAAGGCTCTACAGATTCA
ACTGCTGACTTTGGGTTTGGAGTGTACAATGATCACCATTATCATTTAGGGTACTTTCTT
TATGGAATTGCGGTTCTTGCAAAGATTGATCCTCAGTGGGGACAAAAATACAAGCCACAA
GTTTATTCACTTGTCACAGATTTCATGAACTTGGGCCAGAGATATAACAGATTTTATCCA
CGTCTAAGGTGTTTTGATCTTTACAAATTGCACTCTTGGGCTGCAGGGTTGACCGAGTTT
GAGGATGGAAGGAATCAAGAAAGTACAAGTGAGGCTGTGAATGCATACTATTCAGCAGCA
TTGGTGGGTCTTGCATATGGTGACTCAAGTCTTGTTGACACTGGGTCAACGCTAGTGGCA
TTGGAAATTCTAGCCGCACAAACTTGGTGGCATGTGAAAGTGGAAGACAACTTGTATGAA
GAGGAATTTGCTAAAGATAACAAGATAGTGGGGGTTTTGTGGGCTAACAAGAGGGATAGT
AAGCTATGGTGGGCCAGTGCTGAGTGTAGGGAGTGTAGGCTTGGCATCCAAGTGCTACCC
TTATTGCCTATTACTGAGACATTGTTCTCTGATGCTGATTATGTGAAGGAGCTAGTGGAA
TGGACAGTGCCCTTTTTAAGTAGTCAAGGGTGGAAGGGGATGACCTATGCCCTGCAAGGA
ATTTATGATAAAGAAACAGCATTGGAAAATATAAGAAAGTTGAAAGGTTTTGATGATGGG
AACTCTTTGAGTAATCTCTTGTGGTGGATTCACAGCAGATGA
GmGBP1 CDS4
>G. max Wm82.a4.v1|Glyma.08G245700.1 CDS
SEQ ID NO: 31
ATGTCTTCTTCTTTTCTCTTCCCTCAAACTCAATCCACAGTCCTCCCAGACCCTTCAACC
TACTTCTCACCAAACCTTCTTTCTTCTCCACTCCCCACAAACTCTTTCTTCCAAAACTTT
GTTATTCCAAATGGGACACAGCCTGAGTACATTCACCCCTACCTTATCAAAACCTCAAAC
TCCTCACTCTCAGCCTCATACCCTCTTCTCTTTTTCACCACAGCAGTGTTATACCAAGCT
TTTGTGCCAGATATCACTATCTCTTCCCCTCAAACACACTCACGTCAACAAAACCGTGTA
ATCTCATCATACAGTGACCTTGGTGTCACTTTGGACATTCCCTCTTCAAACCTAAGGTTC
TTTCTCTCAAGAGGAAGCCCTTTTATAACTGCTTCTGTGACAAAGCCAACATCTCTCTCC
ATCACAACAGTGCACACCATTGTCTCTTTGTCTGCCAATGATGACAAAAACACTAAGTAC
ACCCTTAAGCTTAACAACACTCAGGCATGGCTCATATACACCTCCTCCCCAATCTATTTG
AACCATGATGCTGCTTCCAACGTTACATCCAAGCCATTTTCTGGCATAATTCGTGTAGCA
GTGTTGCCTGATTCCAACTCCAAGTGTGTAAAAATTCTCGACAAGTTCAGCTCTTGTTAT
CCTTTGTCGGGTAATGCAACACTCGAGAAGCCTTTCCGTGTGGTGTATGAATGGCTAAAG
GAAGGTTCTGGGAACTTGCTAATGCTAGCTCACCCTCTTCATGTCAAGATTTTATCATCT
ACTAATAATGGTCAAGTTAATGTGCTTCGTCATTTTAAGTATAGAAGCATTGATGGTGAT
CTTGTTGGTGTTGTTGGAGACTCATGGGTGATGGAAACCAATCCTATTCCTGTGACATGG
TATTCTAACAAAGGTGTGGAGAAAGAGTCATATGATGAAATTGTCTCAGCGCTTGTTACG
GATGTGCAAGGGCTGAATTCTTCAGCAATAGAAACAATAATTTCATCTTATTTTTATGGG
AAGCGTGTTGGAAGGGCTGCAAGGTTTGCGTTGATAGCAGAAGAAGTGTCTTTTCCCAAG
GTGATTCCTTCAGTTAAGAAGTTTCTGAAAGAGACTATTGAGCCTTGGTTGGATGGAACT
TTCCCAGGGAATGGTTTTCAATATGAAAATAAATGGGGTGGACTTGTAACCAAACTAGGG
TCAACGGATTCAACCGCTGATTTTGGTTTTGGAATTTACAATGATCACCATTACCATTTG
GGGAACTTCCTTTATGGAATTGCGGTTCTTGCAAAGATTGACCCTCAATGGGGACAAAAG
TACAAGCCACAAGTTTATTCACTTGTGACAGATTTCATGAACTTGGGGCCAAGTTATAAC
AGATTTTATCCACGTCTAAGGAATTTTGACCTTTACAAATTGCACTCTTGGGCTGCAGGG
TTGACTGAATTTGAACATGGAAGGAATCAGGAAAGCACAAGTGAGGCTGTGACTGCTTAC
TATTCTGCAGCGTTGGTGGGTCTTGCATATGGTGACTCAAGTCTTGTTGCCACTGGGTCA
ACGTTAATGGCGTTGGAAATTCTTGCTGCACAAACTTGGTGGCACGTGAAAGAGAAAGAC
AACTTGTACGAAGAAGAATTTGCAAAAGAGAATAGGGTAGTGGGGATTTTGTGGGCTAAC
AAGAGGGATAGTAAGTTATGGTGGGCCAGGGCTGAGTGTAGAGAGTGTAGGCTTGGAATC
CAAGTGCTACCATTGTTGCCTATTACTGAGACATTGTTCTCTGATGCTGATTATGCCAAG
GAGCTTGTGGAATGGACACTGCCTTCTGCACGTAGAGAAGGGTGGAAGGGAATGACATAT
GCCTTGCAAGGAATTTATGATAGGAAAACAGCATTGCAGAATATAAGAATGTTAAAAGGT
TTTGATGATGGGAATTCATTCACTAATCTCTTGTGGTGGATTCATAGCAGATGA
GmGBP1 CDS5
>G. max Wm82.a2.v1|Glyma.18G267100.1 CDS
SEQ ID NO: 32
ATGTCTTCTCCTTCTTCTTTTCTATTCCCTCAAACTCAATCCACAGTCATCCCAGACCCT
TCAACCTACTTCTCACCTAACCTTCTTTCTTCTCCTCTCCCCACAAACTCTTTCTTCCAA
AACTTTGTTATTCCAAATGGGACACAACCTGAGTACATTCACCCTTACCTCATCCAATCC
TCAAACTCTTCACTCTCAGCTTCATACCCTCTTCTCCTCTTCACCACAGCACTGTTGTAC
CAAGCGTTTGTGCCAGATCTCACCATCTCTGCCACTAAAAGATACTCATCATACCAACAA
AACCGTGTAATCTCATCCTACAGTGACCTTGGTGTCACTTTGGACATTCCAAGCTCCAAC
CTTAGGTTCTTTCTTGTCAGAGGAAGCCCTTATATAACTGCTTCTGTCACAAAGCCAACA
CCTCTTTCCATCAAAACAGTGCACACCATAGTTTCTCTGTCTTCCGATGATTCCAACACC
AAGCACACCCTTAAGCTTAACAACACTCAGACATGGATCATATACACGTCCTCCCCAATC
TACTTGAACCATGTTCCTTCTGAGGTTACATCCAAGCCATTTTCCGGCATCATTCGTATA
GCAGCGTTGCCTGATTCTGGTTCCAAGTATGTTGCAACTCTTGACAAGTTCAGTTCTTCT
TACCCTGTGTCTGGTGATGCAGCACTCAAGAAACCATTCCGTCTGGAGTATAAATGGCAA
AAGAAACGTTCTGGGGACTTGCTAATGCTGGCTCACCCTCTTCATGTCAAGCTTCTATCA
TATGATCGTGATGTTACTGTGCTGAATGATTTTAAGTACAGAAGCATTGATGGTGATCTT
GTTGGTGTTGTTGGAGACTCATGGGTGTTGGAAACCAATGCTATTCCTGTGACATGGTAT
TCTAACAAAGGTGTGGACAAAGAGTCTTATGGCGAGATTGTCTCGGCGCTTGTTAAGGAT
GTTCGAGCGCTGAATTCTTCAGCAATAGGAACAAATTCATCTTATTTCTATGGGAAGCAG
GTTGGAAGGGCGGCGAGGTTGGTGTTGATAGCGGAAGAAGTGTCGTATCCTAAAGTGATT
CCAAAGGTTAAGAAGTTTCTGAAGGAGACTATTGAGCCTTGGTTGGATGGAACTTTCAAA
GGGAATGGTTTTCTCTATGAAAGAAAATGGCGTGGACTTGTTACTAAACAAGGCTCTACA
GATTCGACTGCTGATTTTGGGTTTGGAATTTACAATGATCACCATTTCCATTTGGGGTAC
TTCATTTATGGAATTGCAGTTCTTGCAAAGATTGATCCTCAATGGGGACAAAAGTACAAG
CCACAGGTTTATTCACTTGTGACAGATTTCATGAACTTGGGTCAAAGATATAACTCGGAT
TACACACGCCTAAGGTGTTTTGATCTTTATAAGTTACACTCTTGGGCTGCAGGGTTGACT
GAATTTGAAGATGGAAGGAATCAGGAAAGTACAAGTGAAGCAGTGAATGCATACTATGCA
GCAGCATTGATGGGTCTAGCATATGGTGACTCAAGCCTTGTTGCCACTGGATCAACGCTA
GTGGCGTTGGAGATTCTTGCTGCACAAACTTGGTGGCATGTGAAAGCAGAAGACAACTTG
TATGAAGAAGAATTTGCAAAAGATAACAGGATAGTGGGGATTTTGTGGGCTAACAAGAGG
GATAGTAAGCTATGGTGGGCCAGTGCTGAGTGTAGAGAGTGTAGGCTTGGAATCCAAGTG
CTACCCTTGTTGCCTATTACTGAGACATTGTTCTCTGATGCTGATTATGTGAAGGAGCTA
GTGGAATGGACAGTGCCCTTTTTAAGTAGTCAAGGTTGGAAGGGAATGACCTATGCCTTG
CAAGGAATTTATGATAGGGAAACAGCACTGCAGAATATTAGAAAGTTGACAGGTTTTGAT
GATGGGAATTCGTTCACTAATCTCTTGTGGTGGATTCACAGCAGATGA
GmGBP1 CDS6
>G. max Wm82.a2.v1|Glyma.08G246300.1 CDS
SEQ ID NO: 33
ATGTCTTCTTCTTTTCTCTTCCCTCAAACTCAATCCACAGTCATCCCAGACCCTTCAACC
TACTTCTCACCAAACCTCCTTTCATCACCACTCCCCACAAACTCTTTCTTCCAAAACTTT
GTTATTCCTAATGGGACACAGCCTGAGTACATTCATCCTTACCTCATCCAATCCTCAAAC
TCTTCACTCTCAGCCTCATTCCCTCTTCTCCTCTTCACCACAGCACTCCTGTACCAAGCT
TTTGTGCCAGATCTCACTATCTCTGCCTCTAAAACATACTCATCATATCAACAAAACCGT
GTAGTCTCATCCTACAGTGACCTTGGTGTCACTTTGGACATTCCAAGCTCCAACCTTCGA
TTCTTTCTTGTCAGAGGAAGCCCTTATATAACTGCTTCTGTCACAAAGCCAACACCTCTT
TCCATCAAAACAGTGCACACCGTAGTTTCCTTGTCTTCAGATGATTACAACACCAAACAC
ACCCTTAAGCTTAACAACAGTCAGGCATGGATCATATACACCTCCTCTCCAATCTACTTG
AACCATGTTCCTTCTGAGGTTACATCCAAGCCATTTTCTGGCATCATTCGTATAGCGGCT
TTGCCTGATTCTGATTCCAAGTATGTGGAAACTCTTGACAAGTTCAGTTCTTGTTACCCT
GTGTCTGGTGATGCAGCACTCAAGAAACCATTCAGTGTTGAGTATAAATGGCAAAAGAAA
CGTTCTGGGGACTTGCTCATGCTGGCTCACCCTCTTCATGCTAAGCTTCTGTCATATGAT
CGTGATGTTACTGTGCTGAATGATTTTAAGTATAGAAGCATTGATGGTGACCTTGTTGGT
GTTGTTGGAGATTCATGGGTGTTGGAAACCAATCCTATTCCTGTGACATGGAATTCTAAC
AAAGGTGTGGAGAAAGAGTCTTATGGCGAGATTGTCACGGCGCTTGTTAAGGATGTTCAA
GCGCTGAATTCTTCAGCAATAGGAACAAATTCATCTTATTTCTATGGGAAGCAGGTTGGA
AGGGCTGCGAGGTTGGCGTTGATAGCGGAAGAAGTGTCTTACCCTAAAGTGATTCCAAAG
GTTAAGAAATTTCTGAAGGAGACTATTGAGCCCTGGTTGGATGGAACTTTCAAAGGGAAT
GCTTTTCTCTATGAAAGAAAATGGCGTGGACTTGTTACTAAACATGGCTCTACAGATTCA
ACTGCTGATTTTGGGTTTGGAATTTACAATGATCACCATTTCCATTTGGGGTACTTCATT
TATGGAATTGCAGTTCTTGCAAAGATTGATCCTCAATGGGGACAAAAGTACAAGCCACAA
GTTTATTCACTTGTGACAGATTTTATGAACTTGGGCCAAAGATATAACTCAGATTATACA
CGCCTAAGGTGTTTTGATCTTTATAAGTTACACTCTTGGGCTGCAGGGTTGACTGAATTT
GAAGATGGAAGGAATCAAGAAAGTACAAGTGAAGCAGTGAATGCATACTATGCAGCAGCA
TTGCTGGGTCTAGCATATGGTGACTCAAGTCTTGTTGACACTGGATCAACGCTGGTGGCG
TTGGAGATTCTTGCTGCACAAACCTGGTGGCATGTGAAAGCAGAAGACAACTTGTATGAA
GAAGAATTTGCAAAAGATAACAGAATAGTGGGTGTTCTGTGGGCTAACAAGAGGGATAGT
AAACTATGGTGGGCCCCTGCTACGTGTAGAGAGTGTAGGCTTGGAATCCAAGTGCTACCC
TTGTTGCCTATTACTGAGACATTGTTCTCTGATGCTGATTATGTGAAGGAGCTTGTGGAA
TGGACAGTGCCCTTTTTAAGTAGTCAAGGGTGGAAGGGGATGACCTATGCCTTGCAAGGA
ATTTATGATAAGAAAACAGCATTGCAGAATATTAGAAAGTTGACAGGTTTTGATGATGGG
AATTCGTTCACTAATCTCTTGTGGTGGATTCACAGCAGATGA
CcGBP1 CDS1
>C. cajan_rna-KK1_019357_Cc_Asha_v1.0
SEQ ID NO: 34
ATGTCTCCTTCTTTTCTCTTCCCTCAAACACAATCCACAGTCCTCCCTGACCCTTCAACC
TACTTCTCCCCAAACCTTCTTTCTTCTCCACTCCCCACAAACTCTTTCTTCCAAAACTTT
GTTATTCCAAATGGGTCACAGCCTGAGTACATTCACCCTTACCTCATCAAATCCTCAAAC
ACCTCCCTCTCTGCCTCCTACCCATTTCTATTCTTCACTGCAGCAATATTGTACCAGGTT
TTTGTGCCTGATCTCACAATCTCTGCCTCTCGGACATATTCAAATAAACAAAACCGTGTA
GTTTCATCCTATAGTGACCTTGGTGTCACTTTGGACATTCCCTCTTCCAACTTAAGGTTC
TTTCTTGTCAGAGGAAGCCCTTTCATAACTGCTTCTGTGACAAAGCCAACATCTCTATCC
ATCACAACAAATCAAACCATTGTTTCTTTGACTTCCACTAATGACAACACTAAGCACACC
CTTCAGCTCAACAACACTCAGACATGGCTCATATACACCTCCTCACCAATTTATTTGAAT
CATGTTCCTTCTGAGGTCACATCCAAGCCATTTTCTGGCATAATTCGTATAGCAGCGTTG
CCTGATTCCAACCCCAAGAATGTGGAAATTCTTGACAAGTTCAGCTCTTGTTACCCCGTG
TCGGGTGATGCAACACTCAAGAAACCATTCCGTGTGGTGTACAAATGGCAAAAGAAGCAG
TCTGGGGACTTGCTCATGCTAGCTCACCCTCTTCATGCTAAGCTTCTATCATATGATCGT
GAGGTTACTGTTCTGCACGATTTTAAGTATAGAAGTGTCGATGGTGATCTTATTGGTGTT
GTTGGAGATTCATGGGTGTTGGAAACAGATCCTATTCCTGTAACATGGCATTCTAACAAA
GGTATCAAAAAGGAGTCATATGGTGAGATTGTCTCAGCGCTTGTTAAAGATGTAAAGGAG
CTAAATTCTTCTGCAATAACAACAAATTCATCTTATTTCTATGGGAAGCTTGTTGGAAGG
GCTGCAAGGTTGGCATTGATAGCAGAAGAAGTGTCTTTTCCAAAAGTGATTCCCAAGGTT
AGGAAGTTTCTGAAGGAGACTATTGAGCCCTGGTTGGATGGAACTTTCAAAGGGAATGGT
TTTCTATATGAAAGTAAATGGCGTGGACTTGTTACTGAACAAGGCTCTACGGATTCAACT
GCTGATTTTGGGTTTGGAATTTATAACGATCACCATTTTCATTTGGGGTACTTCCTTTAT
GGAATTGCAGTTCTTGCAAAGATTGACCCTGTCTGGGGCCAAAAATACAAATCACAAGCT
TATTCACTTGTGACAGATTTTATGAACTTGGACCAAAGATATAACTCAGATTATCCACGC
CTAAGGAATTTTGACCTTTACAAGTTACACTCTTGGGCATCAGGGGTGACTGAGTTTGAA
GACGGAAGGAATCAGGAAAGTACAAGTGAAGCTGTGAATGCATACTATGCAGCAGGGTTG
ATGGGTCTAGCTTATCGTGATACCGATCTTGTTGCCACTGGATCAACCCTCTTAGCATTG
GAAATTCGTGCTGCACAAACATGGTGGCATGTAAAAGTTGGAGACAACTTGTACGAAGAA
GATTTTGCAAAAGATAACAGGATAGTCGGTGTTCTGTGGGCTAACAAGAGGGACAGTAAG
CTATGGTGGGCTCCTGCTGAGTGTAGAGAATGTAGGCTTAGTATCCAAGTTCTACCCTTG
TTGCCTGTTACTGAGACCTTGTTCTCTGATGCTGTCTATGCGAAGGAGCTTGTGGAGTGG
ACACTGCCTTCTTTGAAGAATAAAACAAATGTAGAAGGCTGGAAGGGATTTACCTATGCC
TTGCAAGGGATTTATGATAAAAATACAGCATTGAAGAAGATAAGAATGTTGAAAGGTTTT
GATGATGGAAACTCGTTCAGTAATCTCCTATGGTGGATTCACAGTAGATGA
CcGBP1 CDS2
>C. cajan_rna-KK1_019354_Cc_Asha_v1.0
SEQ ID NO: 35
ATGTCTTCTCCTTTTGTCTTCCCTGAAACACAATCCACAGTTCTCCCTGACCCTTCAACC
TACTTCTCCCCAAACCTACTTTCTTCTCCGCTCCCCACAAGCTCTTTTTTCCAAAATTTC
GTTATTCCAAACGGGTCACAACCTGAGTACATTCACCCTTATCTCATCAAAACCTCAAAC
ACATCACTTTCTGCCTCATACCCTTTACTCATCTTCACTGCAGCAGTGTTGTACCAAGCT
TTTGTGCCAGATCTCACTATCTCTTCCACTCAAACACAAACAAAAGAACAAAACCGTGTA
GTTTCATCCCACAGTGACCTTGGTGTCACTTTGGACATTCCCTCTTCCAACTTAAGGTTC
TTTCTTTCAAGAGGAAGTCCTTTCATAACTGCTTCTGTGACATCTCCAACGTCTCTCTCC
ATCACAACCAATCACACCATAGCCTCTTTATCTTCCAATGATAACAAAACCAAGCACACC
CTTAGGCTCAACAACACTCAAACATGGCTCATATACACCTCTTCCCCAATCAATTTGAAC
CATGATGATGGTGCTTCCGAGGTTACATCCAAACCATTTTATGGTACAATTCGTCTAGCA
GTGTTGCCTGATTCCAAATATGAGGCAACTCTCGACAAGTTCAGCTCTAGCTACCCTTTA
TCCGGTGATGCAACATTTGAGAATTCGAAGCCTTTTCGTTTGGTGTATCAATGGCAAAAG
AAAGGGTCTGAGAATCTTCTCATGTTAGCTCACCCTCTTCATGTTAAGCTTTTATCAAAG
TACAACAATGCTGGTGTCACTGTGCTTCATGATTTTAAGTATAGAAGCATCGATGGTGAT
CTTGTTGGTGTTGTTGGGGACTCATGGGTATTGGAAATGGATCCTATTCCTGTGACATGG
TATTCTAACAAGGGTGTGAATGATGGTTCACGTGATGAGATTGTGTCAGCGCTTGTTAAG
GATGTGGAAGCGTTGAACTCTTCAGCAATAACAACAAAATCGTCTTATTTCTATGGGAAG
CAAGTTGGTAGGGCTGCGAGGTTGGCATTGATAGCGGAAGAAGTGTCTTTTTCCAAAGTG
GTTCCCACAATTAAGAAGTTTTTGAAAGAGACCATTGACCCTTGGTTGGATGGAACTTTC
AAAGGGAATGGTTTTCTATATGAAAAAAAATGGGGTGGACTAGTAACCAAACTAGGGTCA
ACTGATTCAACAGCTGATTTTGGGTTTGGTGTTTACAATGATCACCACTTTCATTTGGGT
TACTTTCTTTATGGAATTGCGGTTCTAGCAAAGATTGACCCTGAGTGGGGGCAAAAGTAC
AAGCCACAAGCTTATTCACTTGTGACAGATTTTATGAACTTAGACCAAAAGTATAGCACA
ATTTATCCACGTCTAAGGTGTTTTGACCTTTACAAGCTACACTCCTGGGCTTCAGGGGTG
ACCGAATTTGAAGATGGAAGGAATCAAGAAAGTACAAGTGAGGCCGTGAATGCGTACTAT
TCGGCAGCATTGGTGGGTCTAGCATATGATGACTCAAGTCTTGTGGCCACAGGGTCAACG
CTAGTGGCATTGGAGATTCTTGCTGCACAAACTTGGTGGCATGTGAAAGTGGGAGAAAAC
TTGTACCAAGAGGAATTTGCACAAGATAATAGGATAGTGGGCATTTTGTGGGCCAACAAG
AGGGATAGTAAGCTTTGGTGGGCCACTGCTGAGTGTAGAGAGTGTAGGCTTGGGATCCAA
GTGTTACCCTTGCTGCCTATCACTGAGACCTTGTTCTCTGATGCTGTTTATGTTAAGGAG
CTTGTGGAATGGACCATGCCCTATTTGAGTAATGAAGGGTGGAAGGGCATGACCTATGCC
TTGCAAGGGATTTATGATAAGGAAACAGCATTGGATGAGATAAGAAAGTTGAAAGGTTTT
GATGATGGCAACTCTTACACTAATCTCTTGTGGTGGATTCACAGCAGATGA
PIGBP1 CDS1
>P. lunatus_PI08G0000035500.v1
SEQ ID NO: 36
ATGTCTTCTTCTTTTCTATTCCCTCAAACTCAATCCACAGTCATCCCAGACCCTTCAACC
TACTTCTCACCAAACCTCCTTTCATCTCCACTCCCCACAAACTCTTTCTTCCAAAACTTT
GTTATTCCAAATGGTACACTGCCTGAGTACTTTCACCCCTACCACATTCAGTCCTCAAAC
TCTTCACTCTCTGCCTCCTACCCTTTTCTCTTCTTTACAGCAGCTGTGTTGTACCAAGTT
TTTGTCCCAGATCTCACCATCTCTGCCTCTCAAACGTACTCACATGGACAAAACCGTGTA
ATTTCATCCTACAGTGACCTTGGTGTCACTTTGGACATTCCCACTTCCAACCTCAGGTTC
TTTCTTGTCAGAGGAAGCCCTTTCATAACTGCTTCTGTGACAAAGCCAACCTCTCTTTCC
ATCACAACCGTGCACACCATTCTTTCTTTGTCTTCCTATAATGACAATACCAAGTTTATC
CTTCAGCTTAACAACACTCAGACATGGCTCATATACACCTCCTCCCCAATCTATTTGAAC
CATGCTGCTTCTGAGGTTACATCCAAGCCATTTTCTGGCATCATTCGTATAGCAGCGTTG
CCTGATTCCGACCCCAACAATGTCGCAACTCTTGACAAGTTCAGTTCTTGTTACCCTGTG
TCGGGTGATGCAGCACTCAAAAAACCTTTCCGTGTGGAGTATAAATGGCAAAGGAAAAGG
TCAGGGGACTTGCTCATGCTAGCTCACCCTCTTCATGCCAAGCTTCTATCCCATGATTGT
AACGTTACCGTTCTGCACGATTTTAAGTATAGAAGTGTTGACGGTGATCTTGTTGGTGTT
GTTGGAGATTCTTGGGTGTTGGAAACGGATCCTATTCCTGTGACATGGCATTCTAAAAAA
GGCATCAATAAAGAGTCATTTGGTGAGATTGTCTCAGCACTTAATAAGGATGTCAAGGAG
CTAAATTCTTCTGCAATAACAACACAGTCATCTTATTTCTATGGGAAGCTTGTTGGAAGG
GCTGCAAGGTTAGCCTTGATCGCAGAAGAAGTGTCTTATCCTAAAGTGATTCCCAAGATT
ATAAAGTTTTTGAAGGAAACCATTGAGCCCTGGTTGGATGGAACTTTCAAAGGGAATGCT
TTTCTGTATGAAAGAAAATGGCGTGGACTTGTTACTAAACAAGGCTCCACGGATTCAACT
GCTGATTTTGGGTTTGGAGTGTATAACGATCACCATTTTCATTTGGGGTACTTCGTTTAT
GGAATTGCAGTTCTTGCAAAGATTGACCCTGCCTGGGGCAAAAAATACAAACCGCAAGCC
TATTCACTTGTGACAGATTTTATGAACTTGGGCCAAAGATATAACTCAGATTATCCGCGC
CTAAGGTGTTTTGACCTTTATAAGTTACACTCTTGGGCTTCAGGACTGACTGAATTTGAA
GATGGAAGGAATCAGGAGAGTACAAGTGAAGCTGTAAATGCCTACTATGCAGCAGCCTTG
ATGGGTCTAGCTTATGGTGATAGCCGTCTTATTGATACTGGATCGACACTGTTAGCATTG
GAAATTCGTGCTACACAAACATGGTGGCATGTAAAAGCGGAAGACAACTTGTATGAAGAA
GAATTTGCAAAGGATAACAGGATAGTGGGTATTCTGTGGGCTAACAAGAGGGACAGTAAG
CTATGGTGGGCTCCTGCCGAGTGTAGAGAATGTAGGCTTAGTATCCAAGTTCTACCCTTG
TTGCCTGTCACTGAGACCTTGTTCTTTGATACTGTTTATGCGAAGGAGCTTGTGGAATGG
ACACTGCCTTCTTTGAAGAACAAAACAAATGTAGAAGGCTGGAAGGGATTCACCTATGCC
TTGCAAGGAATTTATGATAAAACTACAGCATTAAAGAAAATAAGAATGTTGACAGGTTTT
GATGATGGAAACTCATTCAGTAATCTCCTATGGTGGATTCACAGCAGATAA
PIGBP1 CDS2
>P. lunatus_PI08G0000035600.v1
SEQ ID NO: 37
ATGTCTTCTTCATCTTCTTTTCTCTTCCCTCAAACTCAATCCACAGTTCTTCCAGACCCT
TCAACCTACTTCTCCTCTAACCTTCTTTCATCTCCACTTCCCACAAATTCTTTCTTCCAA
AACTATGTTATCCCAAACGGGTCCCAACCTGAGTACATTCACCCCTACCTCATCAAAACT
ACAAACTCCTCACTATCAGCCTCATACCCTTTTCTCCTCTTCACCACAGCAGTCTTGTAC
CAAGCTTTTGTGCCAGATCTCACCATCTCTTCCACTCAAACACACTCACATCAACAAAAC
CGTGTAATCTCATCATTCAGTGACCTTGGTGTCATTTTGGATATTCCCTCCTCCAACCTG
AGGTTCTTTCTCTCAAGAGGAAGCCCTTTTATAACTGCTTCCGTCACATCTTCAACATCT
CTTTCTATCACAACACTGCACACCATACTCTCTTTATCTTCCAATGATGACGACAACACC
AGGTACACCCTTAAGCTTAACAACTCTCAGACATGGCTCATATACACCTCCTCCCCCATC
CATTTGAACCATAATGCTTCAGAGGTTACGTCCAAGCCATTTTCTGGCATCATTCGTGTA
GCAGTGCTGCCTAATCCTAACTACGAGACAATTCTTGACAAGTACAGCTCTTCTTACCCT
TTGTTGGGTGATGCAACACTAGAGGAGCCTTCCCGTGTGGTGTATCAATGGCAAAAGGAA
GGGTCTGGGGATTTGCTCATGCTGGCTCACCCTCTTCATGTTAAGCTTTTATCAAATAAT
AATGACGGGAATGTTACTTTGCTGAGTGATTTTAAGTATAGAAGCATCGATGGTGATCTT
GTTGGTGTTGTTGGAGATTCATGGATATTGCAAACGGATCGTATTCCTGTGACATGGTAT
TCTAACAACGGAGTGGAAACAAATTCATATGAGGAGATTGTCTCAGCGCTTGTTAAGGAC
GTGCAAGCGCTTAATTCCTCAGCAATAGGAACAAATTCATCTTATTTTTATGGAAAGCGC
GTTGGAAGGGCCGCAAGGTTGGCATTGATAGCGGAAGAAGTGTCTTTTTCAAAGGTTGTT
CCCACGGTTACGGATTTTCTTAAAGGGGCCATTGAGCCTTGGTTAGATGGAACTTTCGAA
GGGAATGGTTTTCTATATGAAAATAAATGGGGTGGACTTGTAACCAAATTAGGATCAACG
GATTCAAGCGCTGATTTTGGGTTTGGAGTTTACAATGATCACCATTACCATTTGGGGTAC
TTTCTATATGGAATTGCGGTTCTTGCAAAGATTGATACCGAGTGGGGACAAAAATATAAG
CCACAAGTTTATTCACTTGTGACAGATTTTATGAACTTGGGTCAAAGGTATAACAGAATT
TATCCACGTCTAAGGTGTTTTGACCTTTATATGTTACATTCTTGGGCTGCAGGAGTAACT
GAATTTGAAGATGGTAGGAATCAAGAAAGTACGAGTGAAGCTGTGAATGCATACTATTCA
GCTGCATTGGTGGGTCTGGCATATGGTGACTCAAGTCTTGTTGCCACTGGGTCAACGTTA
GTGGCGCTGGAAATTCTAGCAGCACAAACTTGGTGGCATGTTAAAGTGGAAGACAACTTG
TACGAAGAAGAATTTGCAAAAGACAATAGGATAGTGGGGATTGTGTGGGCTAATAAGAGG
GATAGCAACTTATGGTGGGCCGGTGCAGACTGTAGAGAATGCAGACTTGGAATCCAAGTG
CTACCCTTGTTGCCTATCACTGAGACATTGTTCTCTGATTCTGATTATGTGAAGGAGCTT
GTGGAATGGACACTTCCCTCTTTAAGTAGTGAGGGGTGGAAGGGAATGACCTATGCCTTG
CAAGGAATTTATGATAAGCAAACAGCATTGCAGAATATAAGAACGTTGAAAGGTTTTGAC
GATGGAAACTCTTACAGTAATCTCTTGTGGTGGATTCACAGCAGATAA
PIGBP1 CDS3
>P. lunatus_PI04G0000054600.v1
SEQ ID NO: 38
ATGTTGAAAAAACTTAGACGCAAGGTTAGCACAGCCCTCAGAAGTGGCCTTAAGAATGGG
TCCAAACCCTATAAAAACCCATCACCACCACCTTTATCACCATTATCACTTCCATTACCA
CTTCCATTAGAACCAGTAAGAACAATGTCTCATACTACAAAACATTCTCCTTTTCTGTTT
CCACATGCTAATTCCTCTGTTGTTCCTGATCCCTCCAATTTCTTCTCCCCAAACCTTCTC
TCAAATCCACTCCCCACTAACTCTTTCTTCCAAAACTTTACCTTGAAAAATGGTGATCAA
CCTGAGTATATTCACCCTTACCTCATCAAATCCTCAAACTTTTCTCTCTCCCTTTCATAC
CCATCTCGCTTTTTTAACTCCTCCTTCACTTACCAGGTCTTCAACCCTGATCTCACCATT
TCTTCCTCTCAAAAGCCCCACCTTTCCCATTTCAACCATACCATCTCTTCCCACAATGAT
CTCAGTGTCACTTTGGACATCCCTTCTTCCAATTTGAGGTTTTTCCTTGTTAGGGGTAGT
CCCTTTTTGACCCTCTCTGTGACTCAACCGACCCCTCTTTCCATCACCACTATTCACGCC
ATTCTATCCTTTTCCTCCAGTGATTCCCTCACAAAGCACACTTTTAACCTCAACAATGGC
CAGACTTGGATTTTGTATGCGTCTTCGCCGATTAGGTTGAGTCATGGACTTTCTGAGATC
AATTCTGATGCGTTTTCTGGCATAATTAGGATTGCCCTGTTGCCTGATTCTGATTCGAAG
CACGAGGCTGTGCTTGACAGGTTCAGTTCCTGTTACCCTGTGAGCGGTGAGGCTGTGTTT
GCCAGGCCATTTTGTGTGGATTATAAGTGGGAGAAGAAAGGATGGGGTGATTTGTTGATG
TTGGCACACCCTCTCCATCTTCAGCTTTTGGCTGATGGTGGTTGTGGAGATGTTAATGTT
CTGAGTGATTTTAAGTACGGGAGCATTGATGGGGACCTTGTTGGGGTTGTTGGTGATTCG
TGGAGTTTGAAAACTGATCCTGTTTCTGTGACTTGGCACTCTATTAGGGGTGTGAGAGAA
GAATCCCGGGATGAGGTTGTTTCGGCGCTTGTGAATGATGTTGAGGGGCTGAATTCATCT
TCAATAACGACGAACTCGTCGTATTTTTATGGGAAACTGATTGCAAGGGCTGCAAGGTTG
GCTTTGATAGCTGAAGAGATGTGCTTTCTTGATGTGATTCCTAAGGTTAGGAAGTATTTG
AAGGAAACCATTGAGCCGTGGCTGGAGGGGACTTTTAATGGGAATGGATTTCTGTATGAT
AGGAAATGGGGGGCATTGTTACCAAACAAGGGTCCAATGATGCTGGTGCTGATTTTGGGT
TTGGAATTTACAATGATCACCATTATCATTTGGGATACTTCGTTTATGGAATTGCAGTGC
TTGCTAAGATTGATCCTGTGTGGGGTAGGAAGTATAAGCCTCAAGCCTATTCTCTCATGG
CAGATTTTATGACATTGAGCAGAAGATCAAATTCGAACTACACAAGACTAAGGTGTTTTG
ACCTTTATAAATTACACTCATGGGCTGGAGGTTTAACTGAGTTTGCAGATGGAAGAAATC
AGGAGAGTACCAGTGAAGCTGTTAATGCATACTATTCTGCTGCCTTGATGGGTCTGGCAT
ATGGTGACACACACCTTGTTGCCACTGGATCAACGCTCACAGCATTGGAGATTCATGCAG
CTCAAATGTGGTGGCATGTGAAACAGGGAGATAATCACTATGGTGAAGGGTTTGAGAAGG
AGAACAAGGTAGTTGGTGTTCTTTGGGCTAACAAGAGGGACAGTGGACTATGGTTTGCGC
CTCCTGAGTGGAAAGAATGTCGGCTTGGGATTCAACTCTTACCGTTACTGCCGATTTCTG
AAGTGTTGTTCTCCGATGTTGATTTTGTGAAGGATCTTGTGGAGTGGACATTGCCTGCCT
TGAACAGGGAAGGTGTTGGAGAAGGATGGAAAGGGTTTGTTTATGCACTGCAGGGAATAT
ATGATAATGAAGGTGCATTGCAGAGGGTAAGAAGCTTGAATGGTTTTGATGATGGAAACA
CATTGACTAATCTATTGTGGTGGATTCACAGCAGAAGTGATGAAGAGGAATTTGGTCATG
GAAAACACTGCTGGTTTGGTCATTACTGCCACTAG
PIGBP1 CDS4
>P. lunatus_PI04G0000054700.v1
SEQ ID NO: 39
ATGTTTAAGAAACTTGGAAGAAAAATTGAAAGAGAAATCACAAAACCCTTCAAAAATAAA
CCACGACCAAGAACATCATCTCCACCTCCACCACCACCTCCTCCTCCACCACCACCTCCT
TCATCTACACCTCCACCGCCACCTCCGCCGCCATCTCCTCCTCCTCTTCCTAAGCAACCA
AATGCTCCATTTCTCTTCCCTCAAGCTCACTCCACAATTCTCCCTGACCCTTCAACTTTC
TTTGCTCCAAACCTTCTCCCTTCTCCACTCCCTACAAACTCTTTCTTCCAAAACTATGTT
CTTCAAAATGGAGATACACCTGAATACATTCACCCCTACCTCATCAAATCCTCAAACTCC
TCCCTCTCCCTCTCCTACCCTTCTCTCAACTTCAACTCTTCTTTCATAGCACAGGTTTTC
AACCCTGACATCACCATCTCTTCCACTGATAGCAAAACCACCCCAGGCTTACACGCGAGC
CACGTGATCTCTTCCTTCAGTGATCTAAGTGTCACTTTGGACATTCCCTCTTCAAACCTC
AGGTTCTTTCTTGTCAGGGGAAGCCCTTTTGTGACAGCATCAGTTACATGTCCCACACCA
CTTTCCATCACCACCATGCATGCCATTCTTTCACTCTCATCCAATAACTCTCTCACCAAA
CACACCTTGCAGCTCAACAATGGCCAATCATGGCTCATTAACACTTCCTCGCCCATCAGT
TTAAACCACAGCCTTTCTGAGATTACTTCTGGTGAATTTTCTGGCATAATTAGGATAGCA
GTGTTGCCTGATTCTGACCCTAAGTATGAGGTAATCCTCAATAGGTTCAGCTCTTGTTAT
CCTGTCTCTGGGGATGCAACATTCACAAATCCGTTCTGTGTAAAGTATAAATGGGAAAAG
AAAGGGTGGGGGGAATTGTTAATGCTGGCTCACCCTCTTCACCTTCAGCTTTTGAATGAT
GGTGATAGTGGTGTGACAGTTCTGCATAATTTAAAATTTAGAAGTATTGATGGAGAGCTT
GTTGGTGTTGTTGGAGACTCCTGGCTGCTGAAAACCGACCCGGTTTCAGTTACTTGGCAT
TCCACAAGAGGTATAAAAGAAGAATTCCATGAAGAGATTTATTCAGTGCTTTCTGAAGAT
GTGGAAGCTTTGAATCCCAAGGGAATAACAACAACATCATGCTATTTTTATGGGAAGATT
ATAGCAAGAGCAGCAAGGTTAGCATTGATAGCTGAAGAGGTGGCTTTTCTCGATGCCATG
CCTGTGATTAGGAAGTTCTTGAAGGAGATCATTGAGCCATGGTTAGACGGTACTTTCAGT
GGAAATGGTTTTCTCTATGAGGGGAAATGGGGAGGGATTGTTACTAAACAAGGGTCTAAA
GATTCAGAAGCAGATTTTGGGTTTGGTGTTTATAATGACCACCATTACAATTTGGGGTAC
TTCCTTTACGGAATTGCGGTGCTTGCAAAGATTGATCCAGCTTGGGGAAGGAAGTACAAG
CCTCAAGCGTATTCACTTGTGGCAGATTTCATGAGCTTGGCAAGAAGATCAGACTCTAAC
TACACGCGTTTGAGGTGTTTTGATCTGTATAAATTGCACTCTTGGGCCGGAGGGTTAACT
GAATTTGCAGATGGAAGAAATCAGGAGAGCACTAGTGAAGCTGTGAATGCATATTATTCT
GCAGCATTGACGGGTCTAGCATATGGTGACACTCAACTTATTGCCACTGGATCAACACTT
GCAGCATTGGAAATTCATGCAGCTCAAATGTGGTGGCATTTGGGAGAGGGAAATAAACTG
TATGAGGAAGATTTTACAAAAGACAACAAGGTGGTAAGTGTTCTGTGGGCTAACAAGAGA
GATAGTGGACTATGGTTTGCTCCTTCTCAGTGGAGAGAATGCAGGCTTGGTATTCATGTT
TTACCACTGTCTCCTATTACGGAGGCCTTGTTCTCAGATGTTGATTATGTGAAGGAACTT
GTGGAGTGGACAGTGCCCAATTTGAACAGGAAGTGTGTTGGAGAAGGGTGGAAGGGGTTT
ATCTATGCCTTGGAAGGAACTTATGATAAAGAAAGTGCACTACAAAAGGTAAGAAGCTTG
AAAGTCTTTGATGATGGGAACTCAATGTCTAATCTGTTGTGGTGGATTCATAGCAGGGGT
GATGTGGAGGAGGAATTTGGTCAAGGAAAACAATGCTGGTTTGGCCATTACTGCCACTAA
PIGBP1 CDS5
>P. lunatus_PI04G0000054500.v1
SEQ ID NO: 40
ATGGTTAAGCAAAAAAAACTCATTTCATCTTCCCAGAGACACAATCCACTGTGCTTCCTG
ATCCCTCCAACTTCTTCTCCTCAACCCTTCTCTCAAAACCACTCCCCACCAACTCTTTCT
TCCAAAACTTTGTCCTAAAAAATGGTGATCAACCTGAATACATTCATCCTTACCTCATCA
AATCCTCTAACTCTTCCCTCTCTCTCTCATACCCTTCTCGCCAAGTCAGTTCTGCTGTCA
TATACCAAGTCTTCAACGCTGATCTCACTATCTCATCCAAGCAAAGTTCCAGTGGGAAAC
ACCTTATCTCCTCCTATAGTGATCTCAGTGTCACTCTGGATATCCCTTCTTCCAATCTTA
GCTTCCTCCTTGTTAGGGGAAGCCCCTTTTTGACTGTTTCTGTCACCCAACCAACCCCTC
TTTCCATCACCACCATCCACACCATTCTCTCATTCTCTTCAAATGAGACTAACACCAAGT
ACACCTTTCAGTTCAACAATGGTCAAACATGGATCCTTTATGCTTCCTCCTCCATCAAGT
TGAGCCACACTCTTTCTGAGATCACTTCTGATACATTTTCTGGCATAGTCCGGATAGCCT
TGTTGCCTGATTCTGATTCAAAACACGAGGCGGTTCTTGACAAGTTTAGTTCTTGTTACC
CCGTGTCTGGTGAAGCTATATTTAGAGAACCCTTTTGTGTGGAGTATAAGTGGGAGAAGA
AAGGGTCAGGAGATTTGCTACTCTTGGCTCACCCTCTCCATGTTCAGCTTTTGTCTAATG
GAGACAATGATGTCACTGTTCTGGAAGATTTTAAGTATGGAAGCATTGATGGGGATGTTG
TTGGTGTTGTTGGGGATTCATGGGTTTTGCAAACAGATCCCGTGTATGTAACATGGCACT
CAACCAAGGGAGTCAAAGAAGAATCCCATGATGAAATTGTTTCAGCCCTTTCGAATGATG
TTGACGGCCTAAACTCATCATCGATTTCAACAACTTCGTCATATTTTTATGGGAAGTTGA
TTGCAAGGGCTGCAAGGTTGGCATTGATTGCTGAGGAGTTGAGCTACCCTGATGTGATTC
CAAAGGTTAAGAAGTTTTTGAAGGAAACCATTGAGCCATGGTTGGTGGGAACTTTCAATG
GGAATGGATTTCTACATGATAAGAAATGGGGTGGCATTATTACCCAACAAGGGTCCAATG
ATGGTGGTGGTGATTTTGGATTTGGTATTTACAATGATCATCACTACCATTTGGGGTACT
TCCTTTATGCAATTGCAGTGCTCGTTAAGCTTGATCCAGCCTGGGGTAGGAAGTACAAGG
CTCAAGCCTATTCCATTGTGCAAGACTTCATGAACTTGGACACTAAACTAAACTCCAATT
ACACACGTTTGAGGTGTTTTGACCTTTATGTGCTTCACTCTTGGGCTGGAGGATTAACTG
AGTTCAGTGATGGAAGGAACCAAGAGAGCACAAGTGAGGCTGTGTGTGCATATTACTCTG
CTGCTTTGATGGGGCTGGCCTATGGTGATGCTCATCTTGTTTCCCTTGGATCAACACTAA
CAGCATTGGAAATTCTTGGGACTAAAATGTGGTGGCATGTGGAAGAGGAAGGGAAATTGT
ATGAGGAAGAGTTCACAAGAGAGAACAGGATCATGGGGGTTCTGTGGTCTAACAAGAGAG
ACACTGGACTATGGTTTGCTCCTGCAGAGTGGAAAGAGTGTAGGCTTGGCATTCAGCTCT
TACCATTGGTACCTATTTCTGAAGCCATTTTCTCCAATGCTGAGTATGTGAAGCAGCTTG
TGGAGTGGACTTTGCCTGCTTTGAATAGGGATGGTGTTGGTGAAGGATGGAAGGGATTTG
TATATGCCCTTGAAGGCATTTATGACAATGAAAGTGCATTGCAGAAGATAAGAAACCTTA
CAGGTTTTGATGGTGGAAACTCTCTCAGTAATCTCTTATGGTGGATTCACAGCATAGGAA
ATGAATAA
PaGBP1 CDS1
>P. acutifolius_Phacu.WLD.008G033800
SEQ ID NO: 41
ATGTCTTCTTCTTTTCTCTTCCCTCAAACTCAATCCACAGTCCTCCCAGACCCTTCAACC
TACTTCTCACCAAACCTCCTTTCATCTCCACTCCCCACAAACTCTTTCTTCCAAAACTTT
GTTATTCCAAATGGTACAGTGCCTGAGTACTTTCACCCCTACCACATTCAGTCCTCAAAC
TCCTCACTCTCTGCCTCCTACCCTTTTCTCTTCTTTACAGCAGCTGTGTTGTACCAAGTT
TTTGTCCCAGATCTCACCATCTCTGCCTCTCAGACATACTCAAATGCACAAAACCGTGTA
ATCTCATCCTACAGTGACCTTGGTGTCACTTTGGACATTCCCACTTCCAACCTCAGGTTC
TTTCTTGTCAGAGGAAGCCCTTTCATAACTGCTTCTGTCACAAAGCCAACCTCTCTTTCC
ATCACAACCGTGCACACTATACTTTCTTTGTCTTCCTATGATGACAACACCAAGTTTATC
CTTCAGCTTAACAACACTCAGACATGGCTCATATACACCTCCTCCCCAATCTATTTGAAC
CATGCTGCTTCTCAGGTTACATCCAAGCCATTTTCTGGCATCATCCGTATAGCAGCTTTG
CCTGATTCCAACCCCAACAATGTCGCAACTCTTGACAAGTTCAGTTCTTGTTACCCTGTG
TCGGGTGATGCAGCACTCAAGAAACCTTTCCGTGTGGAGTATAAATGGCAAAGGAAAAGG
TCAGGGGACTTGCTCATGCTAGCTCACCCTCTTCATGCTAAGCTTCTAGCACATGATTGT
AACGTTACCGTTCTGCACGATTTTAAGTATAGAAGTGTTGACGGTGATCTTGTTGGTGTT
GTTGGAGATTCTTGGGTGTTGGAAACGGATCCTATTCCTGTCACATGGCATTCTAAAAAA
GGCATCGATAAAGAGTCATTTGGAGAGATTGTCTCAGCACTTAATAAGGATGTCAAGGAG
CTAAATTCTTCTGCAATAACAACACAGTCATCTTATTTCTATGGGAAGCTTGTTGGAAGG
GCTGCAAGGTTGGCCTTGATCGCAGAAGAAGTGTCTTATCCTAAAGTGATTCCCAAGATT
ACAAAGTTTTTGAAGGAAACCATTGAGCCCTGGTTGGATGGAACTTTCAAAGGGAATGCT
TTTCTATATGAAAGAAAATGGCGTGGACTTGTTACTAAACAAGGCTCCACGGATTCAACT
GCTGATTTTGGATTTGGAGTGTATAACGATCACCATTTTCATTTGGGGTACTTTATTTAT
GGAATTGCAGTTCTTGCAAAGATTGACCCTGCCTGGGGAAAACAATACAAACCGCAAGCC
TATTCACTTGTGACAGATTTTATGAACTTGGGCCAAAGATATAACTCAGATTATCCGCGC
CTAAGGTGTTTTGACCTTTATAAGTTACACTCTTGGGCTTCAGGGCTGACTGAATTTGAA
GATGGAAGGAATCAGGAGAGTACAAGTGAAGCTGTAAATGCCTACTATGCAGCAGCATTG
ATGGGTCTAGCTTATGGTGATAGCCGTCTTGTTGATACTGGATCGACACTGTTAGCATTG
GAAATTCGTGCTACACAAACATGGTGGCATGTAAAAGTGGAAGACAACTTGTATGAAGAA
GAATTTGCAAAAGATAACAGGATAGTGGGTATTCTGTGGGCTAACAAGAGGGACAGTAAG
CTATGGTGGGCTCCTGCAGAGTGCAGAGAGTGTAGGCTTAGTATCCAAGTTCTACCCTTG
TTGCCTGTCACTGAGACCTTGTTTTTTGATTCTGTTTATGCCAAGGAGCTTGTGGAATGG
ACACTGCCTTCTTTGAAGAACAAAACAAATGTAGAAGGCTGGAAGGGATTCACCTATGCC
TTGCAAGGAATTTATGATAAAACTACAGCATTAAAGAAAATAAGAATGTTGACAGGTTTT
GATGATGGAAACTCATTCAGTAATCTCCTATGGTGGATTCACAGCAGATAA
PaGBP1 CDS2
>P. acutifolius_Phacu.WLD.008G033900_1
SEQ ID NO: 42
ATGTCTTTCTCATCTTCTTTTCTCTTCCCTAAAACTCAATCCATAGTTCTTCCAGACCCT
TCAACCTACTTCTCTTCAAACCTTGTTTCTTCTCCACTCCCCACAAACTCTTTTTTCCAA
AACTTTGTCCTTTTAAACGGGTCACAACCTGAGTACATTCACCCCTACCTCATCCAAACC
TCAAAGTCCTCACTCTCTGCCTCATACCCTCTTCTCTTCTTCACTGCAGCAGTGTTGTAC
CAAACTTTTGTGCCGGATCTCACAATCTCTTCCACTCAAACACTTTCAAATGAACAGAAC
CATGTAATCTCATCCCACAGTGACCTTGGTGTCACTTTGGACATTCCCTCCTCCAACCTC
AGGTTCTTTCTCTCAAGAGGAAGCCCTTTTATAACTGCTTCCGTCACATCTTCAACATCT
CTTTCTATCACAACACTGCACACCATACTCTCTTTCTCTTCCAACAATGAGAACAACACC
AAGTACACCCTTAAGCTTAACAACACTCAGACATGGCTCATATACACCTCCTCCCCCATC
CATTTCAACCATAATGCTTCAGAGGTTACGTCCAAGCCATTTTCTGGCATCATTCGTGTA
GCAGTGCTGCCAAATCCTAACTATGAGACAATTCTTGACAAGTACAGCTCTTGTTACCCT
TTGTTGGGTGATGCAACACTAGAGGAGCCTTCCCGTGTGGTGTATCAGTGGCAAACGGAA
GGTTCTGGGGATTTGCTCATGCTGGCTCACCCTCTTCATGTTAAGCTTTTATCAAATAAT
AATACTGGTACTGTCACTATTTTGCATGATTTTAAGTATAGTAGCATTGATGGTGATCTT
GTTGGCGTTGTTGGAGATTCATGGAAGTTGGAAATGAATCATATTCCTGTAACATGGCAT
TCTAACAAAGGCGTGGAAAAAGAGTCATATGATGAAATTGTCTCAGCACTTTCCAAGGAC
GTTCAAGCACTAAACTCTACACCAATAGCAACAGCATCCTCCTATTTATATGGGAAACTT
ATTGGAAGGGCTGCAAGGTTGGCGTTGATTGCGGAAGAAGTGTCTTTTCCAAACGTGGTT
CCAACGATTAAGGAGTTTCTGAAGGAGAATATTGAGCCTTGGTTGGATGGAACATTCCAA
GGGAACGGTTTTCTATATGAAAATAAATGGGGTGGACTTGTAACAAAACTGGGGTCAACA
GATTCAAGCGCTGATTTTGGGTTTGGAGTGTACAATGATCACCATTACCATTTGGGTTAC
TTTCTTTATGGAATTGCGGTTCTAGCAAAGATTGACCTTGAGTGGGGACAAAAATACAAG
CCACAAGTTTATTCACTTTTGTCAGATTTTATGAATTTGGACCACCAACATAACGCTTAT
TATCCACGTCTAAGGTGTTTTGACCTCTACATGTTACATTCTTGGGCTTCAGGGTTGAAA
GAATTTGCAGATGGACGGAACCAAGAAAGTACAAGTGAAGCTGTGAATGCGTACTATTCA
GCAGCTTTGGTGGGTCTAGCATATGGTGACTCAAGTCTTGTTGCCACTGGGTCAACGTTA
GTGGCGTTGGAAATTCTTGCTGCACAAACTTGGTGGCATGTGAAAGTGGGAGAGAAGTTG
TACAAAGAAGATTTTGCAAAAGACAATAGGATAGTTGGTGTTCTGTGGGCTAATAAGAGA
GATAGTGGACTATGGTGGGCGAGTGCAGAGTGTAGAGAATGCAGACTTGGAATCCAAGTG
CTACCCTTGTTGCCTATCACTGAGACATTGTTCTCTGATGCTGATTATGTGAAAGAGCTT
GTGGAATGGACACTTCCCTCTTTAAGTAGTGAGGGGTGGAAGGGAATGACCTATGCCTTG
CAAGGAGTTTATGATAAGCAAACAGCATTGCAGAATATAAGAACATTGAAAGGTTTTGAT
GATGGAAACTCTTACAGTAATCTCTTGTGGTGGATTCACAGCAGATAA
PaGBP1 CDS3
>P. acutifolius_Phacu.WLD.008G033900_2
SEQ ID NO: 43
ATGTCTTCTTCATCTTCTTTTCTCTTCCCTCAAACTCAATCCACAGTTCTTCCAGACCCT
TCAACCTACTTCTCCTCTAACCTTCTTTCATCTCCACTTCCCACAAACTCTTTCTTCCAA
AACTATGTTATCCCAAACGGGTCACAACCTGAGTACATTCACCCTTACCTCATCAAAACT
ACAAACTCCTCACTATCAGCCTCATACCCTCTTCTCCTCTTCACCACAGCACTCTTGTAC
CAAGCTTTTGTGCCAGATCTCACCATCTCTTCAACTCAAACACACTCACACCAACAAAAC
CGTGTAATCTCATCATTTAGTGACCTTGGTGTCACTTTGGATATTCCCTCCTCCAACCTC
AGGTTCTTTCTCTCAAGAGGAAGCCCTTTTATAACTGCTTCCGTCTCTTCTTCAACATCT
CTTTCTATCACAACACTGCACACCATACTCTCTTTGTCTTCCAACAATGACAACAACACC
AAGTACACCCTTAAGCTTAACAACACTCAGACATGGCTCATATACACCTCCTCCCCCATC
CATTTCAACCATAATGCTTCAGAGGTTACGTCCAAGCCATTTTCTGGCATCATTCGTGTA
GCAGTGCTGCCAAATCCTAACTACGAGACAATTCTTGACAAGCACAGCTCTTGTTACCCT
TTGTTGGGTGATGCAACACTAGAGGAGCCTTCCCGTGTGGTGTATCAATGGCAAAAGGAA
GGGTCTGGGGATTTGCTCATGCTGGCTCACCCTCTTCATGTTAAGCTTTTATCAAATAAT
AATAACGGGAATGTTACTTTGCTGAGTGATTTTAAGTACAGAAGCATTGATGGTGATCTT
GTTGGTGTTGTTGGAGATTCATGGATATTGCAAACGGATCGTATTCCTGTGACATGGTAT
TCTAACAACGGAGTGGAAAAAAATTCATATGATGAGATTGTCTCAGCGCTTGTTAAGGAC
GTGCAAGCGCTTAATTCTTCAGCAATAGGAACAAGTTCATCTTATTTTTATGGAAAGCGC
GTTGGAAGGGCCGCAAGGTTGGCATTGATAGCGGAAGAAGTGTCGTTTTCACAGGTTGTT
CCCACGGTTACGGATTTTCTTAAAAAGGCCATTGAGCCTTGGTTAGATGGAACTTTCGAA
GGGAACGGTTTTCTATATGAAAATAAATGGGGTGGACTTGTAACCAAACTGGGGTCAACG
GATTCAAGCGCTGATTTTGGGTTTGGAGTTTACAATGATCACCATTACCATTTGGGGTAC
TTTCTATATGGAATTGCGGTTCTTGCAAAGATTGATCCCGAGTGGGGACAAAAATACAAG
CCACAAGTTTATTCACTTGTGACAGATTTTATGAACTTGGGTCAAAGGTATAACAGAAAT
TATCCACGTCTAAGGTGTTTTGACCTTTATATGTTACATTCTTGGGCTGCGGGAGTGACT
GAATTTGAAGATGGTAGGAATCAAGAAAGTACGAGTGAAGCTGTGAATGCATACTATTCA
GCAGCGTTGGTGGGTCTGGCATATGGTGACTCGAGTCTTGTTGCCACTGGGTCAACGTTG
GTGGCGTTGGAAATTCTAGCTGCACAAACTTGGTGGCATGTGAAAGTGGAAGACAACTTG
TACGAAGAAGAATTTGCAAAAGACAATAGGATAGTGGGGATTGTGTGGGCTAATAAGAGG
GATAGTAAGTTATGGTGGGCCGGTGCAGACTGTAGAGAATGCAGACTTGGAATCCAAGTG
CTACCCTTGTTGCCTATCACTGAGACACTGTTCTCTGATTCTGATTATGTGAAGGAGCTT
GTGGAATGGACATTTCCCTCTTTAAGTAATGAGGGGTGGAAGGGAATGACCTATGCCTTG
CAAGGAGTTTATGATAAGCAAACAGCATTGCAGAATATAAGAACGTTGAAAGGTTTTGAT
GATGGAAACTCTTACAGTAATCTCTTGTGGTGGATTCACAGCAGATGA
PaGBP1 CDS4
>P. acutifolius_Phacu.WLD.004G045300
SEQ ID NO: 44
ATGTTTAAGAAACTTGGAAGAAAGATTGAAAGAGAAATCACAAAACCCTTCAAAAATAAA
CCACGACCAAGACCATCATCTCCACCTCCACCACCTCCTCCTCCTCCACCACCACTTCCT
TCATCTACACCTCCACCGCCACCTCCGCCGCCATCTCCTCCTCCTCCTCTTCCTAAGCAA
CCAAATGCTCCATTTCTCTTCCCTCAAGCTCACTCCACAATTCTCCCTGACCCTTCAACC
TTCTTTGCTCCAAACCTTCTCTCTTCTCCACTCCCTACAAACTCTTTCTTCCAAAACTAT
GTTCTTCAAAATGGAGACACACCTGAATACATTCACCCCTACCTCATCAAATCCTCAAAC
TCCTCCCTCTCCCTCTCCTACCCTTCTCTCAACTTCAACTCTTCTTTCATAGCACAGGTT
TTCAACCCTGACATCACCATCTCTTCCACTGAGAGCAAAACCACCCCAGGCTTACACGCC
AGGCACGTCATCTCTTCCTTCAGTGATCTAAGTCTCACTTTGGACATTCCCTCTTCAAAC
CTCAGGTTCTTTCTTGTCAGGGGAAGCCCTTTTGTGACAGCATCAGTTACATGTCCCACA
CCACTTTCCATCACCACCATGCATGCCATTCTTTCACTCTCATCCAATAACTCCCTCACC
AAACACACCTTGCAGCTCAACAATGGCCAATCATGGCTCATTAACACCTCCTCGCCCATC
AGTTTAAACTACAGCCTTTCTGAGATTACTTCTGGTGAATTTTCTGGCATAATAAGGATA
GCAGTGTTGCCTGATTCTGACCCTAAGTATGAGGTAATCCTCAATAGGTTCAGCTCTTGT
TATCCTGTCTCTGGGGATGCAACATTCACAAATCCGTTCTGTGTAAAGTATAAATGGGAA
AAGAAAGGGTGGGGGGAGTTGTTAATGCTAGCTCACCCTCTTCACCTTCAGCTTTTGAAT
GATGGTGGTGATAGTGGTGTGACAGTTCTGCATAATTTAAAATTTAGAAGTATTGATGGA
GAGCTTGTTGGTGTTGTTGGAGACTCCTGGCTGCTGAAAACCGACCCGGTTTCAGTTACT
TGGCATTCCACAAGAGGAATAAAAGAAGAATTCCATGAAGAGATTTTTTCAGTGCTTTCT
GAAGATGTGGAAGCTTTGAATCCCTTGGGAATAACAACAACAGCATGCTATTTTTATGGG
AAGATTATAGCAAGGGCAGCAAGGTTAGCATTGATAGCTGAAGAGGTGGCTTTTCTCGAT
GCCATGCCTGTGGTTAGGAAGTTCTTGAAGGAGATCATTGAGCCATGGTTAGACGGAACT
TTCAGTGGAAATGGTTTTCTCTATGAGGGAAAATGGGGAGGGATTGTTACTAAACAAGGG
TCTAAAGATTCAGGAGCAGATTTTGGGTTTGGTGTTTATAATGATCATCATTACAATTTG
GGGTACTTCCTTTATGGAATTGCGGTGCTTGCAAAGATTGATCCAGCTTGGGGAAGGAAG
TACAAGCCTCAAGCCTATTCACTTGTGGCAGATTTCATGAGCTTGGGAAGAAGATCAGAC
TCTAAGTACACGCGTTTGAGGTGTTTTGATCTGTATAAATTGCACTCTTGGGCCGGAGGG
TTAACTGAATTTGCAGATGGAAGAAATCAGGAGAGTACTAGTGAAGCTGTGAATGCATAT
TATTCTGCAGCATTGATGGGTCTAGCATATGGTGACACTCAACTTATTGCCTCTGGATCA
ACACTTGCAGCATTGGAAATTCATGCAGCTCAAATGTGGTGGCATTTGGGAGAGGGACAT
AAACTGTACGAGGAAGATTTTACAAAAGAGAACAAGGTGGTAAGTGTTGTGTGGGCTAAC
AAGAGAGATAGTGGACTATGGTTTGCTCCTTCTCAGTGGAGAGAATGCAGGCTTGGTATT
CATGTTTTACCACTGTCTCCTATTACCGAGGCCTTGTTCTCTGATGTTGGTTATGTGAAG
GAACTTGTGGAGTGGACAGTGCCCAATTTGAACAGGAAATGTGTTGGAGAAGGGTGGAAG
GGGTTTATCTATGCCTTGGAAGGAACTTATGATAAAGAAAGTGCAGTGCAAAAGGTAAGA
AGCTTGAAAGTTTTTGATGATGGGAACTCAATGTCTAATCTGTTGTGGTGGATTCATAGC
AGGGGTGATGTGGAGGAGGAATTTGGTCAAGGAAAACAATGCTGGTTTGGCCATTACTGC
CACTAA
PaGBP1 CDS5
>P. acutifolius_Phacu.WLD.004G045200
SEQ ID NO: 45
ATGTTGAAAAAACTTAGACGCAAGGTTAGCACAGCCCTGAGAAGTGGCCTTAAGAATGGG
TCCAAACCCTATAAAAACCCATCACCACCACCTTCATCACCATTACCACTTCCATTAGTA
CCAGTAAGAACAATGTCTCATACTAGAAAACATTCTCCTTTTTTGTTTCCACATGTTGAT
TCCTCTGTTGTTCCTGATCCCTCCAATTTCTTCTCCCCAAACCTTCTCTCAAATCCCCTC
CCCACTAACTCTTTCTTCCAAAACTTTACCTTGAAAAATGGTGATCAACCTGAGTATTTT
CACCCTTACCTCGTCAAATCCTCAAACTTTTCTCTCTCCCTTTCATACCCATCTCGCTCT
TTTAACTCCTCCTTCACTTACCAGGTCTTCAACCCTGATCTCACCATTTCTTCCTCTCAA
AAGCCCCACCTTTCCCATTTCAACCATACCATCTCTTCCCACAATGATCTCAGTGTCACT
TTGGACATCCCTTCTTCCAATTTGAGGTTTTTCCTTGTTAGGGGTAGCCCCTTTTTGACC
CTCTCTGTGACTCAACCGACCCCTCTTTCCATCACCACTATTCACGCCATTCTATCCTTT
TCCTCCAGTGATTCCCTCACAAAGCACACTTTTAACCTCAACAATGGCCAGACTTGGATT
TTGTATGCTTCTTCGCCGATTAGGTTGAGTCATGGACTTTCTGAGATAAATTGTGATGCG
TTTTCTGGCATAATTAGGATTGCCCTGTTGCCTGATTCTGATTCGAAGCACGAGGCTGTG
CTTGACAGGTTCAGTTCCTGTTACCCTGTGAGCGGTGAGGCTGTGTTTGCCAGGCCATTT
TGTGTGGAGTATAAGTGGGAGAAGAAAGGGTGGGGTGATTTGTTGATGTTGGCACACCCT
CTCCATCTTCAGCTTTTGGCTGATGGTGGTTGTGATGTTAATGTTCTGAGTGATTTTAAG
TATGGGAGCATTGATGGGGACCTTGTTGGGGTTGTTGGTGATTCATGGAGTTTGAAAACT
GATCCTGTTTCTGTGACTTGGCACTCTATAAGGGGTGTGAGAGAAGAATCCCGGGATGAG
GTTGTTTCGGCGCTTGTGAATGATGTTGAGCGGCTGAATTCATCTTCAATAACGACGAAC
TCGTCGTATTTTTATGGGAAACTGATTGCAAGGGCTGCAAGGTTGGCTTTGATAGCTGAA
GAGATGTGTTTTCTTGATGTGATTCCTAAGGTTAGGAAGTATTTGAAGGAAACCATTGAA
CCGTGGCTGGAGGGGACTTTTAATGGGAATGGATTTCTGTATGATAGGAAATGGGGTGGC
ATTGTTACCAAACAAGGGTCCAATGATGCTGGTGCTGATTTTGGGTTTGGAATTTACAAT
GATCACCATTATCATTTGGGATACTTCGTTTATGGAATTGCAGTGCTTGCTAAGATTGAT
CCAGTGTGGGGTAGGAAGTATAAGCCTCAAGCCTATTCTCTCATGGCAGATTTTATGACA
CTGAGCAGAAGATCAAATTCGAACTACACAAGACTAAGGTGTTTTGACCTTTATAAATTA
CACTCATGGGCTGGAGGGTTAACTGAGTTTGCAGATGGAAGAAATCAGGAGAGTACCAGT
GAAGCTGTCAATGCATACTATTCTGCTGCCTTGATGGGTCTGGCATATGGTGACACACAC
CTTGTTGCCACTGGATCAACGCTCACAGCATTGGAGATTCATGCAGCTCAAATGTGGTGG
CATGTGAAACAGGGAGATAATCACTATGGTGAAGAGTTTGAGAGGGAGAACAAGGTAGTT
GGTGTTCTTTGGGCTAACAAGAGGGATAGTGGACTATGGTTTGCGCCTCCTGAGTGGAAA
GAATGTCGGCTTGGGATTCAACTCTTACCGTTACTGCCGATTTCTGAAGTGTTGTTCTCC
GATGTTGACTTTGTGAAGGATCTTGTGGAGTGGACATTGCCTGCCTTGAATAGGGAAGGT
GTTGGAGAAGGATGGAAAGGGTTTGTTTATGCACTGCAGGGAATATATGATAATGAAGCA
GAAGTGATGAAGAGGAATTTGGTCATGGAAAACACTGCTGGTTTGGTCATTACTGCCACT
AGGCAGCTGTTACCTGATGAATGCCCTCTATAG
PaGBP1 CDS6
>P. acutifolius_Phacu.WLD.004G045100
SEQ ID NO: 46
ATGGTTAAGCAAAACAAAACTCATTTCATCTTCCCAGAGACACAATCCACTGTGCTTCCT
GATCCCTCCAACTTCTTCTCCTCAACCCTTCTCTCAAAACCACTCCCCACCAACTCTTTC
TTCCAAAACTTTGTCCTAAAAAATGGTGATCAACCTGAATACATTCATCCTTACCTCATC
AAATCCTCTAACTCTTCCCTCTCTCTCTCATACCCTTCTCGCCAAGTCAGTTCTGCTGTC
ATATTCCAAGTCTTCAACGCTGATCTCACTATCTCATCCAAGCAAGGTTCCAGTGGGAAA
CACGTTATCTCCTCCTATAGTGATCTCAGTGTCACTTTGGATATCCCTTCTTCCAATCTT
AGCTTCCTCCTTGTTAGGGGAAGCCCCTTTTTGACTGTTTCTGTCACCCAACCAACCCCT
CTTTCCATCACCACCATCCACGCCATTCTCTCATTCTCTTCAAACAAGACTAACACCAAG
TACACCTTTCACTTCAACAATGGCCAAACATGGATCCTTTATTCTTCCTCCACCATCAAG
TTGAGCCACACTCTTTCTGAGATCACTTCTGATGCATTTTCTGGCATAGTCCGGATAGCC
CTGTTGCCTGATTCTGATTCAAAACACGAGGCGGTTCTTGACAAGTTTAGTTCTTGTTAC
CCCGTGTCAGGTGAAGCTATATTTAGAGAACCCTTTTGTGTGGAGTACAAGTGGGAGAAG
AAAGGATCAGGAGATTTGCTACTCTTGGCCCACCCTCTCCATGTTCAGCTTCTGTCCAAT
GGAGACAATGATGTTACTGTTCTGGAAGATTTTAAATATGGAAGCATTGATGGGGATGTT
GTTGGTGTTGTTGGGGATTCGTGGGTTTTGCAAACAGATCCCGTGTATGTAACATGGCAC
TCAACCAAGGGAGTCAAAGAAGAATCCCATGATGAAATTGTTTCAGCCCTTTCTAATGAT
GTTGAAGGCCTAAACTCATCATCGATTTCAACAACTTCGTCATATTTTTATGGGAAGTTG
ATTGCAAGGGCTGCAAGGTTGGCATTGATTGCTGAGGAGTTGAGCTACCCTGATGTGATT
CCAAAGGTTAAGAAGTTTTTGAAGGAAAGCATTGAGCCATGGTTGGAGGGAACTTTCAAT
GGGAATGGATTTCTACATGATAAGAAATGGGGTGGCATTATTACCCAACAAGGGTCCAAT
GATGGTGGTGGTGATTTTGGATTTGGTATTTACAATGATCACCACTACCATTTGGGGTAC
TTCCTTTATGCAATTGCAGTGCTCGTTAAGCTTGATCCAGCCTGGGGTAGGAAGTACAAG
GCTCAAGCCTATTCCATTGTGCAAGACTTCATGAACTTGGACACAAAACTAAACAACAAT
TACACACGTTTGAGGTGTTTTGACCTTTATGTGCTTCACTCTTGGGCTGGAGGGTTAACT
GAGTTCAGTGATGGAAGGAACCAAGAGAGCACAAGTGAGGCTGTGTGTGCATATTACTCT
GCTGCTTTGGTGGGGCTGGCATATGGTGATGCTCATCTTGTTTCCCTTGGATCAACACTA
ACAGCATTGGAAATTCTTGGGACTAAAATGTGGTGGCATGTGGAAGAGGAAGGGAGTTTG
TATGAGGAAGAGTTCACAAGAGAGAACAGGATCATGGGGGTTCTGTGGTCTAACAAGAGG
GACACTGGACTATGGTTTGCTCCTGCAGAGTGGAAAGAGTGTAGGCTTGGCATTCAGCTC
TTACCATTGGTACCTATTTCTGAAGCCATTTTCTCCAATGCTGAGTATGTGAAGCAGCTT
GTGGAGTGGACTTTGCCTGCTTTGAATAGGGATGGTGTTGGTGAAGGATGGAAGGGATTT
GTATATGCCCTTGAAGGGATTTATGACAATGAAAGTGCATTGCAGAAGATAAGAAACCTT
GCAGGTTTTGATGGTGGAAACTCTCTCAGTAATCTCTTGTGGTGGATTCACAGCATAGGA
AATGAATGA
CaGBP1 CDS1
>C. arietinum_NC_021161.1
SEQ ID NO: 47
ATGTCATCTTCTTCTGTTCCTTTCCTCTTTCCCCAAACTCATTCCACAATCCTCCCAAAC
CCATCAAACTTCTTCTCACCAAATCTGCTATCCACACCCCTCCCTACAAACTCTTTCTTC
CAAAACTTTGTTCTTCAAAATGGTGACCAACCTGAATACATTCACCCTTACCTCATCAAA
TCCTCAAACTCTTCTCTCTCATTTTCATACCCTCTTCTCTTATTCACAACATCATTTTTA
TACCAAGTTTTTGTTCCAGATCTCACTATTTCTTCCTCACAAAAAACAACATCAAAAAAC
AAACATGTTATTTCATCATATAGTCATCTTAGTGTGACTCTTGAAATCCCTTCTTCAAAT
TTAAGATTTTTTCTTGTTAGAGGAAGCCCTTTTATAACTGCAAATGTTACAAAACCAACT
TCACTTTCAATCACAACACTAAATAAAATAGTTTCTTTCTCTTCTTTTGATTACAAAAAA
ACCAAACACACCCTTCAACTCAATAACACTCAAAAATGGATTATATACACTTCTTCACCA
ATCAATTTCAACCATGATGGTTTTGAGGTTATATCGAATCCATTTTCGGGTATTATTCGT
ATCGCGATTGTTCCTAATTCAAATCCTTTTTATGAGAAAACTCTTGATAAGTTCAGTTCT
TCTTATCCTGTTTCTGGTGATGCAAACATTAAGAAAAATTTTAGTTTGGTTTATAATTTT
CAAAAGAAAAGGTTGGGTGATTTACTTATGCTAGCTCATCCTCTTCATGTTAAGCTTCTA
TCAAATGATGTTAAAGTTTTGCATGATTTTAAGTATAAAAGTGTTGATGGTGATCTTGTT
GGTGTTGTTGGAGATTCATGGTTATTGAAAAATGATCCTGTTTCTGTGAATTGGTATTCT
AATAAAGGTGTTGCAAAAGAATCACATAATGAGATTGTTTCAGCTCTTATTAAAGATGTG
AATGAGTTGAATTTGTCGTCGATTTCAACAACTTCATCTTATTTTTATGGAAAGATTGTT
GGTAGAGCTGCAAGATTTGCTTTGATAGCTGAAGAAGTTTCTTATCTTAAAGTGATTCCA
AAGATTAAATTTTTTTTGAAGGAAACTATTGAGCCATGGTTGAATGGAAATTTCAAAGGA
AATGGTTTTTTATATGAGAAAAAATGGGGTGGACTTGTTACTCAACAAGGGTTAAATGAT
TCAAGTGCTGATTTTGGTTTTGGAGTTTACAATGATCATCATTACCATTTGGGTTATTTT
CTTTATGGAATTTCAGTTCTTGTAAAAATTGATCCTTTATGGGGACAAAAGTATAAACCT
CAAGTTTATTCACTTTTGAAAGATTTTATGAATTTGGGTGAGAGAGATAATAAAAATTAT
CCAAGTTTAAGGTGTTTTGATCATTACAAGTTACATTCTTGGGCTTCAGGGTTGACTGAA
TTTGAAAATGGAAGGAATCAAGAAAGTTCAAGTGAAGCTGTGAATGCATATTATTCAGCT
GCATTAATAGGTTTAGCATATGGTGATTCAAAAATTGTTGAAATTGGGTCAACAATTTTA
GCATTTGAAATTAAGGCTGCACAAACTTGGTGGCATGTGAAATTGGAAAATAATTTGTAT
GGTGAAGATTTTGCAAAAGAGAATAGAATAGTTGGTATTTTATGGGCTAATAAAAGAGAT
AGTAAACTTTGGTGGGCCCCATCTGAATGTAGAGAGTGTAGACTTAGTATACAAGTTTTA
CCTTTGTTGCCAATTAGTGAAACTTTGTTTTTTGATGGTGTTTATGCTAAGGAGTTAGTT
GAATGGACATTACCTTCTTTGAAGAATAAAACTAATGTTGAAGGGTGGAAAGGGTTCACT
TATGCTTTGGAAGGGATTTATGATAAGGAAATAGCATTGAAGAATATTAGAGGATTGAAA
GGTTTTGATGATGGAAACTCATTTACTAATCTTTTGTGGTGGATTCATAGTAGATGA
CaGBP1 CDS2
>C. arietinum_NW_004515975.1_1
SEQ ID NO: 48
ATGTCTACTATCAAAAAGAACACTCCTTTTATCTTCCCACAGACAAATTCAACTGTCCTC
CCTGACCCCTCCAACTTCTTCTCTCCAAATTTGCTATCCACACCCCTCCCTACAAACTCT
TTCTTCCAAAACTTTTCTCTCAAAAATGGTGACCAACCTGAATACATTCATCCTTACCTC
ATCAAATCATCAAACTCATCACTTTCTGTCTCATACCCTTCTCATTTTTCCAATTCATCT
TTCATATACCAAGTTTTCAATGCTGATCTCACCATAACTTCCTTAGAACAAAAAACCAAT
CAAACTTCCAATGAAAAACACATAATATCTTCTTATAGTGATCTTAGTGTCACCTTAGAT
ATCCCTTCATCAAATCTAAGTTTCTTTCTTGTTAGAGGAAGTCCTTATTTAACTTTTTCT
GTAACAAAACCAACACCTCTTTCCATTTCCACCATTCATGCCATTGAATTCTTAGTCCCT
ACAGATCCATCCATTACCAGGTACACCTTTCAGCTTAACAATGGTCAAACATGGCTTTTA
TATGCTTCCTCGCCGATCAAGTTGAGCCATGATCTTTCTGAGATCACCTGTGAGCCTTTT
TCTGGTGTAATTCGGATCGCTTTGTTGCCGAATAACGATCGTAAAATTGAAGATGTTCTT
GAGAAGTATAGTTCTTGTTACCCTTTATCAGGTGATGCTTTTCTTAGAGAACCATTTTGC
GTTGAGTATAAATGGCAGAAGAATGGTTCAAGTGATTTGCTACTATTAGCACACCCTCTT
CATGTTAAGCTTTTGTCTAATAGTGAAAGTGATGTTACTTTTTTGAATGATTTGAAGTAT
ACAAGCATTGATGGTGATCTTGTTGGTGTTGTTGGTGATTCATGGATTTTGAAAACAGAA
CCTGTTTCAATAACTTGGCACTCAAGCAAAGGTGTAAAAGAAGAATCGCATGACGAAATT
GTTTCATCGCTTTCGAAAGATGTTGAAGGTTTAAACTCATCAGCAATAACAACAACATCA
TCATATTTTTATGGAAAATTGATTGCAAGAGCAGCTAGGCTTGCATTGATTGCTGAAGAA
GTTTTTTTCTTTGATGCCATTCAAAAAGTTAGGAACTTTTTGAAGGAAACAATTGAACCA
TGGCTTGAAGGAACTTTCAATGGAAATGGATTTCTATATGATAGAAAATGGGGTGGCATT
ATAACTCAACAAGGGTCTAATGATAGTAATGGCGATTTCGGTTTCGGAATTTACAATGAT
CATCATTATCATTTAGGATATTTTCTTTATGCAATTGCTGTTCTTGTTAAGATTGATCCA
ACATGGGGTAGGAAGTATAAAACTCAAGCTTATTCACTTATGGAAGATTTTATGAACTTG
AACATAAGATTAAACTCGAATTATACGCGGTTAAGGTGTTTCGATCTTTACAAGTTACAT
TCTTGGGCTGGAGGGTTAACTGAGTTTTCTGATGGAAGGAATCAAGAGAGTACTAGTGAA
GCTGTGAATGCATACTATGCTGCAGCATTGATGGGAATGGCATATGGTGATGCTTCACTT
GTGAGCATTGGATCAACCTTAACATCATTGGAAATTCTTGGAACAAAAATGTGGTGGCAT
GTGAAAAAGGAAGGGAAATTGTATGAAGAAGAGTTTACAAAAGAGAATAGGATAATGGGA
GTTTTGTGGTCTAATAAGAGAGATAGTGGACTTTGGTTTGCAGCGGCTGAGTCTAGAGAA
GCTAGGCTTGGAATTCAGCTTATACCATTGAGTCCAATTTCTGAAGTTTTGTTTTCTGAT
GTTAGTTATGTGAAGGATCTTGTGGAGTGGACTTTGCCTGCTTTGAATAGGGAAGGTGTT
GGTGAAGGATGGAAGGGTTTTTTGTACTCATTGCAAGGTGTTTATGATAATCAAGGTGCA
TTGGAGAAGATAAGAAATTTGAATGGTTTTGATGGTGGTAACTCTTTGACTAATCTTTTG
TGGTGGATTCATAGCAGAGGTGAAGATGGTGATGATGAGTAA
MtGBP1 protein
>MtGBP1 (MtrunA17_Chr7g0218781/Medtr7g013170)
SEQ ID NO: 49
MSSSSSLPFLFPQTHSTVLPNPSNFFSQNLLSTPLPTNSFFQNFVLHNGDTPEYIHPYLI
KSSNFSLSISYPLLLFSATMLYQVFSPDLTISSSQKSHTNTTKNHVISSYSDLGVTLDIP
SSNLRFFLVRGSPFITASVTKPTSLSITTLHNIVSLSSFDDKNTKHTLQLNNTQKWIIYT
SSPIKFNHDGSEIVSNPFSGIIRIIVIPNTKFEKILDKFSSCYPVSGDANIKNKFHLEYK
WQKKCSGDLLMLAHPLHVKLLSQSNNVNVTVLHDLKYTSVDGDLVGVIGDSWILETDPVN
VTWYSSKGVTKESHDEIVSALVKDVKELNSSAITTNGSYFYGKIVSRAARFALIAEEVSY
PKVIPIIKNFLKETIEPWLNGTFKGNGFLYEKKWGGLVTKQGVNNSVVDFGFGIYNDHHY
HLGYFLYGIAVLAKIDPFWGQKYKPQAYSLLQDFMNLGQRDNKNYPTLRCFDFFKLHSWA
AGVTEYENGRNQESSSEAVNAYYSAALIGLAYGDKDLVAIGSTLLALEINATQTWWHVKV
ENNLYGEEFAKENRIVGILWANKRDSKLWWAPSECRGCRVSIQVMPLLPITESLFNDGVY
AKELVEWTLPSLKNDTNDDRWKGFIYSLQGIYDKENALKKIRMLEGFANGNSFSNLLWWW
VIHSR*
MsGBP1 protein1
>M. sativa_MS.gene057477.t1
SEQ ID NO: 50
MSSSSSLPFLFPQTHSTVLPNPSNFFSQNLLSTPLPTNSFFQNFVLYNGETPEYIHPYLI
KSSNFSLSVSYPLLLFSTAMLYQVFSPDLTISSSQKTHTNIPKNHVISSYSDLGVTLDIP
SSNLRFFLVRGSPFITASVTKPTPLSITTIHSIISLSPFDKKKTKYTLQLNNNQKWIIYT
SSPIKFNHDGSEVMSNPFSGIIRIVIVPNSKYEQVLDKFSTCYPVSGDANIKNKFHLEYK
WQKKCSGDLLMLAHPLHVKLLSQSNDASVTVLHDLKYTSIDGDLVGVIGDSWILETNPVN
VTWYSSKGVTKESHDEIVSALVKDVKELNSSAITTNGSYFYGKIVSRAARFALIAEEVSY
PKVIPIIKNFLKETIEPWLNGTFKGNGFLYEKKWGGLVTQQGVNDSGVDFGFGIYNDHHY
HLGYFLYGIAVLAKIDPFWGQKYKPQTYALVKDFMNLGQRDNKNYPTLRCFDFFKLHSWA
AGVTEYENGRNQESSSEAVNAYYSAALIGLAYGDKDLVDIGSTLLAFEINATQTWWHVKV
EKNLYGEEFAKENRIVGILWANKRDSKLWWAPSECRGCRVSIQVMPLLPITESLFNDGVY
AKELVEWTLPSLKNETNDDRWKGFIYALQGIYDKENALKKIRMLESFANGNSFSNLLWWI
HSR*
MsGBP1 protein2
>M. sativa_MS.gene97210.t1
SEQ ID NO: 51
MSSSSSLPFLFPQTHSTVLPNPSNFFSQNLLSTPLPTNSFFQNFVLHNGETPEYIHPYLI
KSSNFSLSVSYPLLLFSTAMLYQVFSPDLTISSSQKTHTNIPKNHVISSYSDLGVTLDIP
SSNLRFFLVRGSPFITASVTKPTPLSITTIHSIISLSPFDKKKTKYTLQLNNNQKWIIYT
SSPIKFNHDGSEVMSNPFSGIIRIVIVPNSKYEQVLDKFSTCYPVSGDANIKNKFHLEYK
WQKKCSGDLLMLAHPLHVKLLSQSNDASVTVLHDLKYTSIDGDLVGVIGDSWILETNPVN
VTWYSSKGVTKESHDEIVSALVKDVKELNSSAITTNGSYFYGKIVSRAARFALIAEEVSY
PKVIPIIKNFLKETIEPWLNGTFKGNGFLYEKKWGGLVTQQGVNDSGVDFGFGIYNDHHY
HLGYFLYGIAVLAKIDPFWGQKYKPQTYALVKDFMNLGQRDNKNYPTLRCFDFFKLHSWA
AGVTEYENGRNQESSSEAVNAYYSAALIGLAYGDKDLVDIGSTLLAFEINATQTWWHVKV
EKSLYGEDFAKENRIVGILWANKRDSRLWWAPSECRGCRLSIQVMPLLPITESLFNDGVY
AKELVEWTLPSLKNETNDDRWKGFIYALQGIYDKENALKKIRMLEGFANGNSLSNLLWWI
HSR*
MsGBP1 protein3
>M. sativa_MS.gene91658.t1
SEQ ID NO: 52
MSSSSSLPFLFPQTHSTVLPNPSNFFSQNLLSTPLPTNSFFQNFVLHNGETPEYIHPYLI
KSSNFSLSVSYPLLLFSTAMLYQVFSPDLTISSSQKTHTNIPKNHVISSYSDLGVTLDIP
SSNLRFFLVRGSPFITASVTKPTPLSITTIHSIISLSPFDKKKTKYTLQLNNNQKWIIYT
SSPIKFNHDGSEVMSNPFSGIIRIVIVPNSKYEQVLDKFSTCYPVSGDANIKNKFHLEYK
WQKKCSGDLLMLAHPLHVKLLSQSNDASVTVLHDLKYTSIDGDLVGVIGDSWILETNPVN
VTWYSSKGVTKESHDEIVSALVKDVKELNSSAITTNGSYFYGKIVSRAARFALIAEEVSY
PKVIPIIKNFLKETIEPWLNGTFKGNGFLYEKKWGGLVTQQGVNDSGVDFGFGIYNDHHY
HLGYFLYGIAVLAKIDPFWGQKYKPQTYALVKDFMNLGQRDNKNYPTLRCFDFFKLHSWA
AGVTEYENGRNQESSSEAVNAYYSAALIGLAYGDKDLVAIGSTLLAFEINATQTWWHVKV
EKYLYGEEFAKENRIVGILWANKRDNNLWWAPSECRGCRLSIQVMPLLPITESLFNDGVY
AKELVEWTFPSLKNETNDDRWKGFIYALQGIYDKENALKKIRMLEGFANGNSFSNLLWWI
HSR*
MsGBP1 protein4
>M. sativa_MS.gene021861.t1
SEQ ID NO: 53
MSSSSSLPFLFPQTHSTVLPNPSNFFSQNLLSTPLPTNSFFQNFVLHNGETPEYIHPYLI
KSSNFSLSVSYPLLLFSATMLYQVFSPDLTISSSQKTHTNIPKNHVISSHSDLGVTLDIP
SSNLRFFLVRGSPFITASVRKPTSLSITTLHNIVSLSSFDDKNTKYTLHLNNTQQWIIYT
SSPIKFNHDGSEIVSNPFSGIIHIVVVPSSKYEKILDKLSSCYPVSGDANIKNRFHLEYK
WKKKCSGDLLMLAHPLHVKLLSQSNNVNVTVLHDLKYTSVDGDLVGVIGDSWILKTDPVN
VTWYSSKGVTKESHDEIVSALVNDVKELNSSAITTNGSYFYGKIVSRAARFALIAEEVSY
PKVIPIIKNFLKETIELWLNGTFKGNGFLYEKKWGGLVTKQGVNNSGVDFGFGIYNDHHY
HLGYFLYGIAVLAKIDPFWGQKYKPQIYALVKDFMNLGQRDNKNYPTLRCFDFFKLHSWA
AGVTEYENGRNQESSSEAVNAYYSAALIGLAYGDKDLVAIGSTLLAFEINATQTWWHVKV
ENNLYGEEFAKENRIVGILWANKRDSKLWWAPSECRGCRVSIQVMPLLPITETLFNDGVY
AKELVEWTLPSLKNETNDDRWKGFIYALQGIYDKGNALKNIRMLEGFANGNSFSNLLWWI
HSR*
MsGBP1 protein5
>M. sativa_MS.gene069419.t1
SEQ ID NO: 54
MSSVPFLFPQTHSTVLPNPSNFFSQNLLSTPLPTNSFFQNFVLQNGDQHEYIHPYLVKSS
NFSVSVSYPLLLFSTAMLYQVFSPDLTISSSQKTHTNIPKNHVISSYSDLGVTLDIPSSN
LRFFLVRGSPFITASVTKPTPLSITTIHSIISLSPFDKKKTKYTLQLNNNQTWIIYTSSP
INLNHDGSEVKSGPFSGIIRIAVVPDSNGEKILDKFSSCYPVSGDANIKKKFGLVYKWQR
KNSGDLLMLAHPLHVKLLSKSNNHGVTVLDDFKYKSVDGDLVGVVGNSWNLKTDSVNVTW
HSNKGVTKESHAEIVSALVNDVKKLNFSSITTNSSYFYGKIVGRAARFAFIAEEVSYPKV
IPIIKNFLKETIEPWLDGNFKGNGFFYEKSWGGFVTQQGINDSSADFGFGIYNDHHYHLG
YFLYGIGVLAKIDPSWGQKYKPQVYSLVKDFMNLGQRDNKNYPTLRCFDPYKLHSWASGL
TEFEHGRNQESSSEAVNAYYSVALVGLAYGDKDLVATGSTLLALEINAVQTWWHVKFENN
LYGGDFAKGNRIVGILWSNKRDSALWWAASECRECRLSIQVLPLLPITESLFNDGVYAKE
LVEWTVPSFKNKTNIEGWKGFTYALQGVYDKKNALKNIRMLKGFDDGNSFSNMLWWIHSR*
MsGBP1 protein6
>M. sativa_MS.gene021900.t1
SEQ ID NO: 55
MSSVPFLFPQTHSTVLPNPSNFFSQNLLSTPLPTNSFFQNFVLQNGDQHEYIHPYLVKSS
NSSLSVSYPLLLFSTAMLYQVFSPDLTISSSQKTHTNIPKNHVISSYSDLGVTLDIPSSN
LRFFLVRGSPFITASVTKPTPLSITTIHSIISLSPFDKKKTKYTLQLNNNQTWIIYTSSP
INLNHDGSEVKSGPFSGIIRIAVVPDSNGEKILDKFSSCYPISGDANIKKKFGLVYKWOR
KNSGDLLMLAHPLHVKLLSKSNNHGVTVLDDFKYKSVDGDLVGVVGNSWNLKTDSVNVTW
HSNKGVTKESHAEIVSALLNDVKKLNISSITTNSSYFYGKIVGRAARFALIAEEVSYPKV
IPIIKNFLKETIEPWLDGNFKGNGFFYEKSWGGLVTQQGINDSSADFGFGIYNDHHYHLG
YFLYGIGVLAKIDPSWGQKYKPQVYSLVKDFMNLGQRDNKNYPTLRCFDPYKLHSWASGL
TEFEHGRNQESSSEAVNAYYSVALVGLAYGDKDLVATGSTLLALEINAVQTWWHVKVENN
LYGQDFAKENRIVGILWANKRDSALWWAASECRECRLSIQVLPLLPITESLFNDGVYAKE
LVEWTVPSFKNKTNIEGWKGFTYALQGVYDKKNALKNIRMLKGFDDGNSFSNMLWWIHSR*
MsGBP1 protein7
>M. sativa_MS.gene91618.t1
SEQ ID NO: 56
MLYQVFSPDVTISSSQKTHTNIPKNHVISSYSDLGVTLDIPSSNLRFFLVRGSPFITASV
TKPTPLSITTIHSIISLSPFDKKKTKYTLQLNNNQTWIIYTSSPINFNHDGSEVKSGPFS
GIIRIAVVPDSNGEKILDKFSSCYPVSGDANIKKKFGLVYKWQRKNSGDLLMLAHPLHVK
LLSKSNNHGVTVLDDFKYKSVDGDLVGVVGNSWNLKTDSVNVTWHSNKGVTKESHAEIVS
ALVNDVKKLNFSSITTNSSYFYGKIVGRAARFALIAEEVSYPKVIPIIKNFLKETIEPWL
DGNFKGNGFFYEKSWGGLVTQQGINDSSADFGFGIYNDHHYHLGYFLYGIGVLAKIDPSW
GQKYKPQVYSLVKDFMNLGQRDNKNYPTLRCFDPYKLHSWASGLTEFEHGRNQESSSEAV
NAYYSVALVGLAYGDKDLVATGSTLLALEINAVQTWWHVKVENNLYGQDFAKENRIVGIL
WANKRDSALWWASSECRECRLSIQVLPLLPITESLFNDGVYAKELVEWTVPSFKNKTNIE
GWKGFTYALQGVYDKKNALKNIRMLKGFDDGNSFSNMLWWIHSR*
MsGBP1 protein8
>M. sativa_MS.gene44625.t1
SEQ ID NO: 57
MLYQVFSPDLTISSSQKTHTNIPKNHVISSYSDLGVTLDIPSSNLRFFLVRGSPFITASV
TKPTPLLITTIHSIISLSPFDKKKTKYTLQLNNNQTWIIYTSSPINFNHDGSEVKSGPFS
GIIRIAVVPDSNGEKILDKFSSCYPISGDANIKKKFGLVYKWORKNSGDLLMLAHPLHVK
LLSKSNNHGVIVLDDFKYKSVDGDLVGVVGNSWNLKTDSVNVTWHSNKGVTKESHAEIVS
ALVNDVKKLNFSSITTNSSYFYGKIVGRAARFALIAEEVSYPKVIPIIKNFLKETIEPWL
DGNFKGNGFFYEKSWGGLVTQQGINDSSADFGFGIYNDHHYHLGYFLYGIGVLAKIDPSW
GQKYKPQVYSLVKDFMNLGQRDNINYPTLRSFDPYKLHSWASGLTEFEHGRNQESSSEAV
NAYYSVALVGLAYGDKDLVATGSTLLALEINAVQTWWHVKVENNLYGQDFAKENRIVGIL
WANKRDSALWWASSECRECRLSIQVLPLLPVTESLFNDGVYAKELVEWTVPSFKNKTNIE
GWKGFTYALQGVYDKKNALKNIRMLKGFDDGNSFSNLLWWIHSR*
PsGBP1 protein1
>Psat3g201680
SEQ ID NO: 58
MSSPPYLFPQTQSTILPNPSNFFSPNLLSTPLPTSSFFQNFALKNGDQPEYIHPYLIQSSN
SSLSVSYPLLLFSTALLYQVFSPDLTISSTQKPQTNIPQNNHVISSYSDLGVTLDIPTAN
LRFFLVRGSPFVTALVTKPTPLSIKTNHTIVSFSSFDYKKTKYRLSLNNGQKWIIYTSSP
INFNHDGSEVKSDPFSGIIRFAVVSNSNNEKILHEFSSSYPVSGYAKIEDKFGLVYKWKT
KNSGDLLMLAHPLHVKLLSKNSNDHKVTILNDFKYRSVDGDLVGVVGKSWLLKTDSVNVT
WHSSKGVSKDSYEEVVSALEKDVNELNVATINTTSSYFYGKIVARAARLALIAEEVSYEK
VIPIVKDFLKKTIEPWLDGNFKGNGFLYEKTWGGLVTQQGVNDSGADFGFGVYNDHHYHL
GYFLYGIGVLAKLDQDWGQKYKPIVYSLLKDFMNLGQRDNKNYPTLRSFDPYKLHSWASG
LTEFRDGRNQESTSEAVNAYYSVTLVGLAYDDEDLVAIGSTLLAFEINAAQTWWHVKAEN
NVYGTDFAKQNPVVGVLWANKRDSSLWWASSECRQCRLSIQVLPLLPITENLFNDGVYAK
ELVEWTWPTLSKEGWKGFTYALQGVYDKENALKNIRTLKGFDDGNSLSNLLWWIHSR*
PsGBP1 protein2
>Psat3g201640
SEQ ID NO: 59
MCSPPYLFPQTQSTILPNPSNFFSQDLLSTPLPTNSFFQNFALKNGDQPEYIHPYLIQSS
NSSLSVSFPLLFFSTALLYQVFTPDLTISSTQKPQTNIPQNNHVISSYSDLGVTLDIPTT
NLRFFLVRGSPFVTAQVTKPTPLSIKTIHAILSFSSFDNKKNKYALSLNNGQKWIIYTSS
PINFNHDISEVKSDPFTGVIRIAAVSDSNNEKILDEFSSSYPVSGHAIVDVKNKFGLVYK
WETENSGDLLMLAHPLHVKLLSKNSNDHKVTILNDFKYRSVDGDLVGVVGNSWLLKTDTI
NVTWHSSKGVAKESYEEVVSALEKDVNELNVASISTTSSYFYGKIVARAARFALIAEEVS
YEKVIPIVKDFLKKTIEPWLDGNFKGNGFLYEKTWGGLVTQQGVNDAGADFGFGVYNDHH
YHLGYFLYGVGVLAKLDQDWGQKYKPIVYSLLKDFMNLGQGDNKNYPTLRSFDPYKLHSW
ASGLTEFSDGRNQESTSEAVNAYYSAALVGLAYGDEDLVAIGSTLLALEINAAQTWWHVK
TENNVYGADFAKQNSVVGVLWANKRDSSLWWASSECRECRLSIQVLPLLPITENLFNDGV
YVKELVEWTWPTLSNEGWKGFTYALQGVYDKENALNNIRALKGFDDGNSLSNILWWIHSR*
VfGBP1 protein
>V. faba_jg123098.t1
SEQ ID NO: 60
MSSPPYLFPQTQSTILPNPSNFFSQNLLSTPLPTNSFFQNFALKNGDQPEYIHPYLIQSS
NSSLSVSYPLLLFSTALLYQVFSPDLTISSTQQPQTNINHVISSYSDLGVTLDIPTSNLR
FFLVRGSPFVTALVTKPTPLSIKTIHTIVSFSTFDNKKTKYTLSLNNTQKWIIYTSSPIN
FNHLGSEVISDPFSGIIRIASVSNSNNEKILDEFSSSYPVSGYAKIENKFGLVYKWETQN
SGDLLMLAHPLHAKLLSNSKDHKVTILNDFKYRSIDGDLVGVVGNSWLLKTDSFNVTWHS
SKGVTKESYEEVVSALEKDVNELNVASITTTSSYFYGKIVARAARFALIAEEVSYEKVIP
VVKGFLKQTIEPWLDGKFKGNGFLYEKTWGGLVTQQGVNDVGADFGFGVYNDHHYHLGYF
LYGIGVLAKIDQDWGQKYKPIVYSLLKDFMNLGLGDNPNYPTLRNFDPYKLHSWASGLTE
FRDGRNQESTSEAVNAYYSVTLIGLAYGDEDLVVVGSTLLALEINAAQSWWHVKAENNVY
GTDFAKQNPIVGVLWANKRDSSLWWASSACRECRLSIQVLPLLPITENLFNDGVYAKELV
EWTLPTLSNEGWKGFTYALQGVYDKENALKNIRTLKGFDDGNSLSNLLWWIHSR*
TpGBP1 protein
>T. pratense_Tp57577_TGAC_v2_mRNA26446
SEQ ID NO: 61
MSSVPFLFPQTHSTVLPNPSNFFSQNLLSTPLPTNSFFQNFVLKNGDQPEYIHPYLIKSS
NFSLSVSYPFLLFSTAMLYQVFSPDLTISSSQKSHTNSQKNKHFISSYSDLGVTLDIPSS
NLRFFLVRGSPFVTASVTKPTPLSITTLHNIVSLSCFDNKKTKYTLLLNNTQKWIIYTSS
PINLNHDGSEVKSGPFSGIIRIAVVPDSNYEKILDKFSSCYPVSGYANIQKKFGLVYKWQ
RKNSGDLLMLAHPLHVKLLSKSNNHGVTVLNDFKYRSVDGDLVGVVGNSWNLKTDPIDVT
WHSSKGVTKESHDEIVSALVKDVKKLNISAIETNSSYFYGKIVGRAARFALIAEEISYFK
VIPIIKNFLKKTIEPWLDGNFKGNGFFYEKSWGGLVTQQGINDSSADFGFGVYNDHHYHL
GYFLYGIGVLAKIDPLWGQKYKPIVYSLLKDFMNLGKRDNKNYPTLRCFDPYKLHSWASG
VTEFENGRNQESSSEAVNAYYSAALVGLAYNDKNLVATGSTLLALEINAVQTWWHVKAES
NLYGEDFAKENRIVGILWANKRDSKLWWAPSECRECRLSIQVLPLLPITETLFNDGVYAK
ELVEWTLPSLKNKTNVEGWKGFTYALQGVYDNKNALKKIRLLKGFDDGNSFSNLLWWIHS
R*
TrGBP1 protein1
>T. repens_CM019102.1
SEQ ID NO: 62
MSSVPFLFPQTHSTVLPNPSNFFSQNLLSTPLPTNSFFQNFVLNNGDQPEYIHPYLIKSS
NSSLSVSYPFLLFSTAMLYQVFSPDLTISSSQKSHSNSPKNKHVISSYSDHGVTLDIPSS
NLRFFLVRGSPFVTAYVTKPTPLSITTLHNIVSLSSFDNKKTKFTLLLNNTQKWIIYTSS
PINLNHDGSEVKSDPFSGIIRIAVVPDSNYEKILDKFSSCYPVSGYANIQKKFGLVYKWQ
TKNSGDLLMLAHPLHVKLLSKSNNHGVIVLNDFKYRSVDGDLVGVVGNSWNLKTDSIDVT
WHSSKGVTKESHDEIVSALVKDVKELNISSIATNSSYFYGKIVGRAARFALIAEEVSYFK
VIPIIKNFLKETIEPWLDGNFKGNGFFYEKSWGGLVTQQGINDSSADFGFGVYNDHHYHL
GYFLYGIGVLAKIDPLWGQKYKPRVYSILKDFMNLGQRDNKNYPTLRCFDPYKLHSWASG
ATEFENGRNQESSSEAVNAYYSAALVGLAYNDKNLVATGSTLLALEINAAQTWWHVKVEN
NLYGEDFAKENRIVGILWANKRDSKLWWAPSECRECRLSIQVLPLLPITETLFNDGVYAK
ELVEWTLPSLKNKTNVEGWKGFTYALQGVYDKKNALKKIRMLKGFDDGNSFSNLLWWIHS
R*
TrGBP1 protein2
>T. repens_CM019114.1
SEQ ID NO: 63
MSSVPFLFPQTHSTVLPNPSNFFSQNLLSTPLPTNSFFQNFVLNNGDQPEYIHPYLIKSS
NSSLSVSYPFLLFSTAMLYQVFSPDLTISSSQKSHSNSSKNKHVISSYSDLGVTLDIPSS
NLRFFLIRGSPFVTALVTKPTPLSITTLHTIVSLSSFDNKKTKFTLLLNNTQKWIIYTSS
PINLNHDGSEVKSDPFSGIIRIAVVPDSNYEKILDKFSSCYPVSGYANIQKKFGLVYKWQ
TKNSGDLLMLAHPLHVKLLSKSNNHGVIVLNDFKYRSVDGDLVGVVGNSWNLKTDSIDVT
WHSSKGVTKESHDEIVSALVKDVKELNISSIATNSSYFYGKIVGRAARFALIAEEVSYFK
VIPIIKNFLKETIEPWLDGNFKGNGFFYEKSWGGLVTQQGINDSSADFGFGVYNDHHYHL
GYFLYGIGVLAKIDPLWGQKYKPRVYSILKDFMNLGQRDNKNYPTLRCFDPYKLHSWAS*
TsGBP1 protein
>T. subterraneum_Tsud_chr4.g17370.1.am.mk
SEQ ID NO: 64
MSSVPFLFPQTHSTVLPNPSNFFSQNLLSTPLPTNSFFQNFVLNNGDQPEYIHPYLIKSS
NSSLSVSYPFLLFSTAMLYQVFSPDLTISSSQKSHSNSTKNKHFISSYSDLGVTLDIPSS
NLRFFLVRGSPFVTASVTKPTPLSITTLHNIVSLSSFDNKKTKYTLLLNNTQKWIIYTSS
PINLNHDGSEVKSDPFSGIIRFAVVPNSNYEKILDKFSSCYAVSGYANIQKKFGLVYKWQ
RKNSGELLMLAHPLHVKLLSKSNNHGVTVLNDFKYRSVDGDLVGIVGNSWNLKTDSIDVT
WHSSKGVTKESHDEIVAALVKDVKELNISAIETNSSYFYGKIVGRAARFALIAEEVSYFK
VIPIIKNFLKKTIEPWLDGNFKGNGFFYEKSWGGLVTQQGINDSSADFGFGVYNDHHYHL
GYFLYGIGVLAKIDPLWGQKYKPIVYSLLKDFMNLGQRDNKFYPTLRCFDPYKLHSWASG
VTEFENGRNQESSSEAVNAYYSAALVGLAYNDKNLIATGSTLLALEINAAQTWWHVKVEN
NLYGEDFAKENRIVGILWANKRDSKLWWAASECRECRLSIQVLPLLPITETLFNDGVYAK
ELVDWTLPSLKNKTNVEGWKGFTYALQGVYDKKNALKKIRMLKGFDDGNSFSNLLWWIHS
R*
LjGBP1 protein
>L. japonicus_Lj1g3v3023590.1
SEQ ID NO: 65
MIFITNNGSKGNTYARSFILTSKVNFHQSSLVSNLTKNSYKKTTPPHKQLHQHLIFSPPT
TMPPSSPFLFPQTQSTVLPNPSTFFSQNLLSSPLPTNSFFQNLVIQNGSQPEYIHPYLIQ
SSNSSLSASYPLLFFSAALLYQTFVPDLTISSTIKTSNPQNHVISSYSDLGVTLDIPSSN
LRFYLARGSPYITASVTKPTPLSITTVHSIVSLLSAADKTKHTLQLNNNQTWLIYSSAPI
NLNKHGSSELQSDPFSGVIRIAVVPDSTSNPKYEEVLDKFSSCYPVSGDAKLKGNFTVVY
KWQRKNSGDLLMLAHPLHLKLLSKNKLAATVLYDFKYRSVDGDLVGVVGDSWVLEAEPVP
VTWHSNRGIKKESYGEIVSALLKDVKELNYSAVATNSSYFYGKLVGRAARFALIAEEVSF
PKVIPKIVKFLKESIEPWLDGTFKGNGFLYETKWGGLVTQQGSKDAGADFGFGIYNDHHF
HLGYFLYGIAVLAKIDPAWGQKYKPQAYALVNDFMNLGQRYYTFSPRLRCFDPYKMHSWA
SGLTEFENGRNQESTSEAVNAYYSAALMGLAYGDTRLATTGSTLTALEIGATQMWWHVKK
EQILYPEEFAEDNRIVGILWANKRDSNLWWAPAECRECRLSIQVLPLLPVTESLFSDAGY
AKELVEWTLPSLKSKSNVEGWKGFTYSLQGIYDKEIALKSIRMLKGFDDGNSYSNLLWWW
VIHSR*
LAGBP1 protein1
>L. angustifolius_OIW16739
SEQ ID NO: 66
MSSPPFLFSQTQSTVLPNPSTFFSQNLLSSPLPTNSFFQNFVLKNGDQPEYIHPYLIKSS
NSSLSVSYPFLLFTTAMLYQVFVPDLTISTSSSSHKSETKTSHVISSYSDLGVTLDIPSS
YLRFFLVRGSPFITTSVTKPTTLSITTTNKIVSLHSFNDKTKHTLQLQNNQTWLIYTSYP
IVFYHKDYAIESNKFSGIIRFAAWPDSTPKYEEILDKFSSCYPVSGDATIKNPFRVVYKW
QRKRSGELLMLAHPLHVKLLSSSLAFNNVTVLNDFKYRSVDGDLVGVVGDSWVLETEHVP
ITWHSKNGVKKESYNEIVSALFKDVKELNASNVTTNSSYFYGKLVGRAARLALIAEEVSY
LEVIPKISDFLKEMIQPWLDGNFKGNGFLYERKWGGLVTKQGSIDAGADFGFGIYNDHHF
HLGYFLYGIAVLAKIDPAWGQKYKPQAYALVTDFLNLGQRFNSYSPRLRCFDLYKLHSWA
SGITEFEDGRNQESTSEAVNAYYAAALLGLAYRDTRLVATASTLTALEILAAQTWWHVKS
EDKLYDEEFTKDNRIVGILWANKRDSKLWWASSECRECRLSIQVLPLVPVTESLFSDAGY
VKELVEWTLPSLKNKSNVDGWKGFTYALQGIYDKENSLKKIRMLKGFDDGNSFSNLLWWI
HSR*
LaGBP1 protein2
>L. angustifolius_OIW17321
SEQ ID NO: 67
MAAPTPFLFPATQPTILPDPSTFFSSNLSSPLPTNSFFQNFVLNSGEQPEYIHPYLVKST
KNSLSIAYPLLLFTASVFYQTFAPDLTISSATPQESAAKNHVISSYSDLGVTLDIPSSNL
RFFLVRGSPYITASVTKPTTLSIKTTSPIESLNPSKDNTKYILKLKSGQTWIIYSSSAIS
LTKGETEISSNSFSGIIRFASLRNPQQESTLDKYSSSYPVSGYAVFNKSFNVVYNLEKEG
NGDLLLLAHPLHVKLLSSKSNKVTVLSDFKYPSVDGELVGVVGDSWELETKHVPLTWNSV
KGVKKEAYEEIVKALVNDVNELNSSNVTTSSSYFYGKLVARAARLALIAEEVSNSEVIPK
ITKFLKDTIQPWLDGSFKGNSFLYEKKWGGLVTKQGSTDKGADFGFGVYNDHHYHLGYFI
YGIAVLAKIDTAWGQKYKPQAYALVSDFLNTDLKSNSHYPLLRNFDVYKLHSWASGLTEF
ADGRNQESTSEAVNAYYAAALMGVAYHDMDLVRIASTVTALEIHAAQTWWHVKSGDKLYA
EEFAKGNKIVGIVWSNKRDSSLWWASAEAKECRLSIQVLPLSPITEALFSDAAYVKELVE
WTLPSLNKPNIEGWKGFTYALQGIYDKSSSLEKIRALKGVDDGNSFTNLLWWIHSR*
LalbGBP1 protein1
>L. albus_Lalb_Chr10g0092981
SEQ ID NO: 68
MQQSLYKSKKSPLPFHMHILSSISMAHNLQHEPFLFPLTHSTVLPDPSNFFSPNLLSTPL
PTNSFFQNFALKNGDQPEYIHPYLIKSSNSSLSVSYPSHFFTTAFIYQVFIADLTISASV
KTNSDSIHKHVISSYNDLSVTLDFPSSNLRFFLVRGSPFLTANVTSSTPLSITTIHAILS
FSSSDSLTKFTLKLNNSQTWLIYSSSPMKFSHTLSGISSDAFCGVIRIAVLPESKNSKFE
EILDRFSSCYPISGDAILKKPFSVVYKWEKKGLGDLLLLAHPLHLQMLSKKNSDVTILDE
FKYKSIDGDLVGVVGDSWLLKTKPVYVTWHSIQGVKKESYSEIVSALSKDVEGLNSAAIT
TASSYYYGKLVARAARLALIAEEIGFRDAISAITKFLKESIEPWLDGTLEENGFLYDEKW
GGLVTKQGSIDSGADFGFGIYNDHHYHLGYFLYGIAVLVKIDPSWGIKYKPQAYSLMQDF
MNLGEKSNSNYPTLRCFDLYKLHSWAGGLTEFADGRNQESTSEAINAYYSAALLGLAYND
TNIFETASTFASLEIHAAKTWWHVKFGDNLYEEDFTKENRIMGVLWSNKRDSGLWFAPPA
MKECRVGIQLLPLVPISEMLFSNVSFVKELVKWTLLALDRNDVEDEWKGFVYALQGIYDN
ESALQKIRRLKGFDDGNSFTNLLWWIHSR*
LalbGBP1 protein2
>L. albus_Lalb_Chr04g0258421
SEQ ID NO: 69
MSVPTPFLFPSIQSTVLPDPSSFFSPNISSPLPTNSFFQNLVLNGGGQPEYFHPYLINST
KTSLSVAYPLLLFTASVVYQTYVPELTISATSQESATKNHVISSFSDLGVTLDLPSSNLR
FFLVRGSPYITASVTKPTTLSINTSSAIESLSASSHRNTKYILKLKSKQTWIIYSSSPIS
LTNEGTEIRSNSYSGIIRFASLRNPHYESTLDKFSSSYPVSGDAEIKKPFHLRYKWQKKG
NGGLLMLAHPLHVKLLPRLFSHVIVLRDFKYPSVDGDLVGVVGDSWELETKPVPVTWRSV
KSVKKESYQEIVKALVKDVNELNSSNVTSTSSYFYGKLVARAARLALIADEVNNHEVIPK
ISIFLKETIQPWLDGSFKGNAFLYEKRWGGLVTRQGSVDKGADFGFGVYNDHHYHLGYFL
YGIAVLAKIDTAWAKKYKSQTYALVTDFLNTDQRLKQSPRLRNFDLYMLHSWASGLTEFG
DGRNQESTSEAVNAYYAAALVGLAYGDKRLISTASTLTALEIRAAQTWWHVKSKNKVYAE
EFAKGNKIVGVLWSIKRDSGLWWAAAERKECRLSIQVLPLSPITESLFSDPSYVKELVEW
TLPSVESKQNVEGWKGFIYALQGIYDKGKSLEKIRTLKGVDDGNSFTNLLWWIHSR*
VuGBP1 protein1
>V. unguiculata_Vigun05g034200.1
SEQ ID NO: 70
MSSSSSFMFPQTQSTVLPDPSTYFSSNLLSSPLPTNSFFQNFVLSKGSQPEYIHPYLIQT
SKSSLSASYPLLFFTAAVLYQTFVPDLTISSSQTLPTQQNHVVSSFSDLGVTVDIPSSNL
RFFLSRGSPFITASVTSSTSLSITTLHTILSLSPSNDKNTKYTLKLNNTQTWLIYASSPI
YLNRDGASQVTSKPFSGIIRVAALPDDNPNNVAILDKFSSSYPSSGNATLHDPFRLVYQW
QKEGSGDLLMLAHPLHAKLLSHNNTGNVNILRDFKYRSIDGDLVGVVGDSWKLEMNPIPV
TWHSNKGVGKESYNEIVSALSKDVQTLNSPISTPSSYAIGKLIGRAARLALIAEEVSFPN
VVPTIKEFLKRNIQPWLDGTVQGNGFLYEKKWGGLVTKMGSTDSSADFGFGVYNDHHYHL
GYFLYGIAVLAKIDNEWGQKYKPQVYALLSDFMNLEQQNAHYPRLRCFDLYRLHSWASGV
TEFADGRNQESTSEAVNAYYSAALVGVAYGDKSLVSAGSTLLAMEILGTQTWWHVKAEDK
LYNEEFAKNNKIVGVLWSNKRDSGLWWAPATCRECRLGIQVLPLSPITETLFSDAGYVKG
LVEWTLPSLSSEAWKGMTYALQGVYDKQTALQNIRRLKGFDDGNSFTNLLWWIHSR*
VuGBP1 protein2
>V. unguiculata_Vigun05g034300.1
SEQ ID NO: 71
MSSSSSFLFPQTQSTVLPDPSTYFSSNLLSSPLPTNSFFQNYVIPNGSQPEYIHPYLITT
SNSSLSASYPFLLFTTALLYQAFVPDLTISSTQTQSHDQRNRVISSFSDLGITLDIPSSN
LSFFLSRGSPFITASVTSSTSLSITTLHTILSLSPSNDNNTKYTLKLNNTQTWLIYASDP
IYLNRDGASEVTSKPFSGIIRVAVLPDPNYATTLDKFSSCYPLSGDATLKESFRLVYQWE
KEGSGDLLMLAHPLHVKLLSNKSNGQVTVLSDFKYRSIDGDLVGVVGDSWVLETDRIPVT
WYSSKGVEKDSYDEIVSALVKDVEKLNSSAIGTSSSYFYGKRVGRAARLALIAEEVSFSK
VVPTIMDFLKEAIEPWLDGTFVGNGFLYENKWSGLVTKLGSTDSTADFGFGVYNDHHYHL
GYFLYGIAVLAKIDPEWGQKYKPQVYSLVTDFMNLGQRYSRIYPRLRCFDLYMLHSWAAG
VTEFEDGRNQESTSEAVNAYYSAALVGLAYGDSNLVETGSTLVALEILAAQTWWHVKVED
NLYNEEFAKDNRIVGILWANKRDSKLWWASAECRECRLGIQVLPLLPITETLFSDADYVK
ELVEWTLPSLSSEGWKGMTYALQGIYDKETALQNIRTLTGFDDGNSYSNLLWWIHSR*
VuGBP1 protein3
>V. unguiculata_Vigun05g034000.1
SEQ ID NO: 72
MSPSFLFPQTQSTVLPDPSTYFSPNLLSSPFPTNSFFQNFVIPNGTQPEYFHPYHIQASN
SSLSASYPFLFFTAAVLYQVFVPDLTISASQTTSYGQNRVISSYSDLGVTLDIPSSNLRF
FLVRGSPFITASVTKPTSLSIKTVHTILSLSSYDGNTKFIIQLNNTQTWLIYTSSPIYLN
HVPSEVTSKPFSGIIRIAALPDSNPSNVATLDKFSSCYPVSGDATLGKPFRLEYKWQKKR
SGDLLMLAHPLHAKLLSRDCNVTVLHDFKYRSVDGDLVGVVGDSWVLETDPIPVTWHSKK
GISKESFGEIVSALYKDVKGLNSSAITTNSSYFYGKLVGRAARLALIAEEVSYYKVIPKI
RKFLKETIEPWLDGTFKGNGFLYERKWRGLVTEQGSTDSTADFGFGIYNDHHFHLGYFLY
GIAVLAKIDPAWGKKFKPQAYSLATDFMNLGQRYNSDYPRLRCFDLYKLHSWASGLTEFE
DGRNQESTSEAVNAYYAAALMGLAYGDSRLVDTGSTLLALEIRATQTWWHVKAEDNLYEE
EFAKDNRIVGILWANKRDSKLWWATAECRECRLSIQVLPLLPVTETLFSDTVYTKELVEW
TLPSLKNKTNVEGWKGFTYALQGIYDKSTALKQIRRLTGFDDGNSFSNLLWWWIHSR*
PvGBP1 protein1
>P. vulgaris_Phvul.008G033200.1_Pv_G19833_v2.1
SEQ ID NO: 73
MSSSSSFLFPQTQSTVLPDPSTYFSSNLLSSPLPTNSFFQNYVIPNGSQPEYIHPYLIKS
TNSSLSASYPLLLFTTALLYQAFVPDLTISSTQTHSQQQNRVISSFSDLGVTLDIPSSNL
RFFLSRGSPFITASVTSSTSLSITTLYTILSLSSNNENNTKYTLKLNNTQTWLIYTSSPI
HENHNASEATSKPFSGIIRVAVLPNPNYETILDKYSSCYPLLGDATLEEPSRVVYQWQKE
GSGDLLMLAHPLHVKLLSNNNNGNVTLLSDFKYRSIDGDLVGVVGDSWILQTDRIPVTWY
SNNGVETNSYDEIVSALVKDVQALNSSAIGTTSSYFYGKRVGRAARLALIAEEVSFSKVV
PTVTDFLKEAIEPWLDGTFEGNGFLYENKWGGLVTKLGSTDSSADFGFGVYNDHHYHLGY
FLYGIAVLAKIDPEWGQKYKPQVYSLVTDFMNLGQRYNRNYPRLRCFDLYTLHSWAAGVT
EFEDGRNQESTSEAVNAYYSAALVGLAYGDSSLVATGSTLVALEILAAQTWWHVKVEDNL
YEEEFAKDNRIVGIVWANKRDSKLWWAGADCRECRLGIQVLPLLPITETLFSDADYVKEL
VEWTFPSLSSEGWKGMTYALQGVYDKQTALQNIRTLKGFDDGNSYSNLLWWIHSR*
PvGBP1 protein2
>P. vulgaris_Phvul.008G033100.1_Pv_G19833_v2.1
SEQ ID NO: 74
MSFSSSFLFPKTQSTVLPDPSTYFSSNLVSSPLPTNSFFQNFVLLNGSQPEYIHPYLIQT
SKSSLSASYPLLFFTAAVLYQTFVPDLTISSTQTLPNEQNHVISSHSDLGVTLDIPSSNL
RFFLSRGCPFITASVTSSTSLSIRTLHTILSLSSNNENNTKYTLKLNNTQTWLIYTSSPI
HENHNALEVTSKPFSGIIRVAVLPNPNYETILDKYSSCYPLLGDATLEEPSRVVYQWQKE
GSGDLLMLAHPLHVKLLSNNNTGTDTILHNFKYSSIDGDLVGVVGDSWKLEMNHIPVTWH
SNKGVEKESYDEIVSALSKDVQALNSSPIATASSYLYGKLIGRAARLALIAEEVSFPNWV
PTIKEFLKKNIEPWLDGTFQGNGFLYENKWGGLVTKLGSTDSSADFGFGVYNDHHYHLGY
FLYGIAVLAKIDPEWGQKYKPQVYSLLSDFMNLDHQHNAYYPRLRCFDLYMLHSWASGLK
EFADGRNQESTSEAVNAYYSAALVGLAYGDSSLVATGSTLVALEILAAQTWWHVKVGEKL
YKEEFAKDNRIVGVLWANKRDSGLWWASAECRECRLGIQVLPLLPITETLFSDADYVKEL
VEWTLPSLSSEGWKGMTYALQGVYDKQTALQNIRTLKGFDDGNSYTNLLWWIHSR*
PvGBP1 protein3
>P. vulgaris_Phvul.008G033000.1_Pv_G19833_v2.1
SEQ ID NO: 75
MSQFPFSSSQTMSSSFLFPQTQSTVLPDPSTYFSPNLLSSPLPTNSFFQNFVIPNGTVPE
YFHPYHIQSSNSSLSASYPFLFFTAAVLYQVFVPDLTISASQTYSNAQNRVISSYSDLGV
TLDIPTSNLRFFLVRGSPFITASVTKPTSLSITTVHTILSLSSYDDNTKFILQLNNTQTW
LIYTSSPIYLNHAASQVSSKPFSGIIRIAALPDSNPNNVATLDKFSSCYPVSGDAALKKP
FRVEYKWQRKRSGDLLMLAHPLHAKLLSHDCNVTVLHDFKYRSVDGDLVGVVGDSWVLET
DPIPVTWHSKKGIDKESFGEIISALNKDVKELNSSAITTQSSYFYGKLVGRAARLALIAE
EVSYPKVIPKIRNFLKETIEPWLDGTFKGNAFLYERKWRGLVTKQGSTDSTADFGFGVYN
DHHFHLGYFIYGIAVLAKIDPAWGKQYKPQAYSLVTDFMNLGQRYNTDYPRLRCFDLYKL
HSWASGLTEFEDGRNQESTSEAVNAYYAAALMGLAYGDSRLVDTGSTLLALEIRATQTWW
HVKVEDNLYEEEFAKDNRIVGILWANKRDSKLWWAPAECRECRLSIQVLPLLPVTETLFF
DTVYAKELVEWTLPSLKNKTNVEGWKGFTYALQGIYDKTTALKKIRMLTGFDDGNSFSNL
LWWIHSR*
GmGBP1 protein1
>G. max Wm82.a2.v1|Glyma.08G245600.1.p
SEQ ID NO: 76
MSSFLFPQTQSTVLSDPSTYFSSNLLSSPLPTNSFFQNFVIPNGSQAEYIHPYLIKTSNS
SLSASYPLLILFTTAVLYQTFVADITISSTQTTSQNHVISSYSDLGVTLDIPSSNLRFFL
SRGSPFLTVSVTSPTSLSITTVHTIVSLSSNDDNNTKYTLKLNNTQTWLIYTSSPIYFTH
NNASEVTSKPFSGIIRVAVLPNHNYVTILDKFSTCYPLSGNATLVEPFRVVYEWQKEGSG
DLLMLAHPLHVKLLSNNYNGLVTVLNDFKYRSIDGDLVGVVGDSWVLETNPIPVTWYSNK
GMEKDSYDEIVSALVKDVQELNSSSIGTSSSYFYGKRVGRAARLALIAEEVSFSNVVPTI
KKFLKESIEPWLDGTLQGNGFLYENKWGGLVTKLGSTDSTADFGFGVYNDHHYHLGYFLY
GIAVLAKIDPEWGQKYNPQVYSLVTDFMNLGQKYNSRYPRLRCFDLYNLHSWASGVTEFA
DGRNQESTSEAVNAYYSAALVGLAYGDSNLVAIGSTLLALEILAAQTWWHVKAEGNLYEE
EFAKENKIVGVLWANKRDSALWWGPATCRECRLGIQVLPLSPVTETLFSDADYVKELVEW
TMPSLTSEGWKGMTYALQGIYDKETALENIRKLKGFDDGNSLSNLLWWIHSR*
GmGBP1 protein2
>G. max Wm82.a2.v1|Glyma.08G246000.1.p
SEQ ID NO: 77
LPNPSTYFSSNLVSSPLPTNSFFQNFALQNGTQAEYIHPYLIKTSNFSLSASCPLLLFTT
AVLYQTFVADITISSTQTTSQNHVISSYSDLGVTLDIPSSNLRGSPYITASVTKPTSLSI
TTVRSIVSLCSNNKENTKYTLKLNNTQTWLIYTSSPIYLNHDAASNITSKHFLQCCLFPT
SRVWQFSTSSALVTRSLSGNATLVKPFRVTYEWQKKGPGFLLTLAHPLHVKLLQYKKNHR
MIVLRDFKYRSIDGDLVGVVGDSWLLKTDTIPVTWHSNKGVEKESHDEIVSALSKDVEAL
SSSPIATESSYYYGKLIGRAARLALIAEEVSSPNVIPTIQKFLKDSIEPWLDGTFQGNGF
LYENKWGGLVTKQGSTDSGADFGFGVYNDHHYHLGYFLYGIAVLAKVDLQWGQKYKPQVY
SLVSDFMNSGQKYNSHYPRLRCFDLYKLHSWTSGVTEFTDGRNQESTSEAVNAYYSAALV
GLAYDDSNLVATGSTLLALEILAAQTWWHVKAEGNLYEEEFAKENKIVDALWANKRDSAL
WWAPATCRECRLGIEVLPLSPVTETLFYDADYVKELVEWTMPSLTSEGWKGMTYALQGIY
DKETVLQNIRMLTGFDDGNSFTNLLWWIHSR*
GmGBP1 protein3
>G. max Wm82.a2.v1|Glyma.18G266900.1.p
SEQ ID NO: 78
MSSNFLFPQTQSTVLPNPSTYFSSNLLSSPLPTNSFFQNFVIPNGSQAEYIHPYLVKTSN
SSLSASYPLLLFTTALLYQSFVPDITISSTQTHSNQQNREISSYSDLSVTLDIPSSNLRF
FLSRGSPFITASVTSPTSLSITTVHTIVSLSSNDDNNTKYTLKLNNTQIWLIYTSSPIYL
NHDGASNITSKPFSGIIRVAALPDSNSKSVAILDKFSSCYPLSGNATLVEPFRVVYQWQK
ESSGDLLMLAHPLHVKLLSNSQVTVLKDFKYRSIDGDLVGVVGDSWVLETDPIPVTWYSN
KGVDKDSYDEVVSALVKDVQELNSSAIGTSSSYFYGKRVGRAARLALIAEEVSFSNVVPT
IKKFLKESIEPWLDGTFQGNSFLYENKWGGLVTKQGSTDSTADFGFGVYNDHHYHLGYFL
YGIAVLAKIDPQWGQKYKPQVYSLVTDFMNLGQRYNRFYPRLRCFDLYKLHSWAAGLTEF
EDGRNQESTSEAVNAYYSAALVGLAYGDSSLVDTGSTLVALEILAAQTWWHVKVEDNLYE
EEFAKDNKIVGVLWANKRDSKLWWASAECRECRLGIQVLPLLPITETLFSDADYVKELVE
WTVPFLSSQGWKGMTYALQGIYDKETALENIRKLKGFDDGNSLSNLLWWIHSR*
GmGBP1 protein4
>G. max Wm82.a2.v1|Glyma.08G245700.1.p
SEQ ID NO: 79
MSSSFLFPQTQSTVLPDPSTYFSPNLLSSPLPTNSFFQNFVIPNGTQPEYIHPYLIKTSN
SSLSASYPLLFFTTAVLYQAFVPDITISSPQTHSRQQNRVISSYSDLGVTLDIPSSNLRF
FLSRGSPFITASVTKPTSLSITTVHTIVSLSANDDKNTKYTLKLNNTQAWLIYTSSPIYL
NHDAASNVTSKPFSGIIRVAVLPDSNSKCVKILDKFSSCYPLSGNATLEKPFRWVYEWLK
EGSGNLLMLAHPLHVKILSSTNNGQVNVLRHFKYRSIDGDLVGVVGDSWVMETNPIPVTW
YSNKGVEKESYDEIVSALVTDVQGLNSSAIETIISSYFYGKRVGRAARFALIAEEVSFPK
VIPSVKKFLKETIEPWLDGTFPGNGFQYENKWGGLVTKLGSTDSTADFGFGIYNDHHYHL
GNFLYGIAVLAKIDPQWGQKYKPQVYSLVTDFMNLGPSYNRFYPRLRNFDLYKLHSWAAG
LTEFEHGRNQESTSEAVTAYYSAALVGLAYGDSSLVATGSTLMALEILAAQTWWHVKEKD
NLYEEEFAKENRVVGILWANKRDSKLWWARAECRECRLGIQVLPLLPITETLFSDADYAK
ELVEWTLPSARREGWKGMTYALQGIYDRKTALQNIRMLKGFDDGNSFTNLLWWIHSR*
GmGBP1 protein5
>G. max Wm82.a2.v1|Glyma.18G267100.1.p
SEQ ID NO: 80
MSSPSSFLFPQTQSTVIPDPSTYFSPNLLSSPLPTNSFFQNFVIPNGTQPEYIHPYLIQS
SNSSLSASYPLLLFTTALLYQAFVPDLTISATKRYSSYQQNRVISSYSDLGVTLDIPSSN
LRFFLVRGSPYITASVTKPTPLSIKTVHTIVSLSSDDSNTKHTLKLNNTQTWIIYTSSPI
YLNHVPSEVTSKPFSGIIRIAALPDSGSKYVATLDKFSSSYPVSGDAALKKPFRLEYKWQ
KKRSGDLLMLAHPLHVKLLSYDRDVTVLNDFKYRSIDGDLVGVVGDSWVLETNAIPVTWY
SNKGVDKESYGEIVSALVKDVRALNSSAIGTNSSYFYGKQVGRAARLVLIAEEVSYPKVI
PKVKKFLKETIEPWLDGTFKGNGFLYERKWRGLVTKQGSTDSTADFGFGIYNDHHFHLGY
FIYGIAVLAKIDPQWGQKYKPQVYSLVTDFMNLGQRYNSDYTRLRCFDLYKLHSWAAGLT
EFEDGRNQESTSEAVNAYYAAALMGLAYGDSSLVATGSTLVALEILAAQTWWHVKAEDNL
YEEEFAKDNRIVGILWANKRDSKLWWASAECRECRLGIQVLPLLPITETLFSDADYVKEL
VEWTVPFLSSQGWKGMTYALQGIYDRETALQNIRKLTGFDDGNSFTNLLWWIHSR*
GmGBP1 protein6
>G. max Wm82.a2.v1|Glyma.08G246300.1.p
SEQ ID NO: 81
MSSSFLFPQTQSTVIPDPSTYFSPNLLSSPLPTNSFFQNFVIPNGTQPEYIHPYLIQSSN
SSLSASFPLLLFTTALLYQAFVPDLTISASKTYSSYQQNRWVSSYSDLGVTLDIPSSNLR
FFLVRGSPYITASVTKPTPLSIKTVHTVVSLSSDDYNTKHTLKLNNSQAWIIYTSSPIYL
NHVPSEVTSKPFSGIIRIAALPDSDSKYVETLDKFSSCYPVSGDAALKKPFSVEYKWQKK
RSGDLLMLAHPLHAKLLSYDRDVTVLNDFKYRSIDGDLVGVVGDSWVLETNPIPVTWNSN
KGVEKESYGEIVTALVKDVQALNSSAIGTNSSYFYGKQVGRAARLALIAEEVSYPKVIPK
VKKFLKETIEPWLDGTFKGNAFLYERKWRGLVTKHGSTDSTADFGFGIYNDHHFHLGYFI
YGIAVLAKIDPQWGQKYKPQVYSLVTDFMNLGQRYNSDYTRLRCFDLYKLHSWAAGLTEF
EDGRNQESTSEAVNAYYAAALLGLAYGDSSLVDTGSTLVALEILAAQTWWHVKAEDNLYE
EEFAKDNRIVGVLWANKRDSKLWWAPATCRECRLGIQVLPLLPITETLFSDADYVKELVE
WTVPFLSSQGWKGMTYALQGIYDKKTALQNIRKLTGFDDGNSFTNLLWWWIHSR*
CaGBP1 protein1
>C. cajan_rna-KK1_019357_Cc_Asha_v1.0
SEQ ID NO: 82
MSPSFLFPQTQSTVLPDPSTYFSPNLLSSPLPTNSFFQNFVIPNGSQPEYIHPYLIKSSN
TSLSASYPFLFFTAAILYQVFVPDLTISASRTYSNKQNRVVSSYSDLGVTLDIPSSNLRF
FLVRGSPFITASVTKPTSLSITTNQTIVSLTSTNDNTKHTLQLNNTQTWLIYTSSPIYLN
HVPSEVTSKPFSGIIRIAALPDSNPKNVEILDKFSSCYPVSGDATLKKPFRVVYKWQKKQ
SGDLLMLAHPLHAKLLSYDREVTVLHDFKYRSVDGDLIGVVGDSWVLETDPIPVTWHSNK
GIKKESYGEIVSALVKDVKELNSSAITTNSSYFYGKLVGRAARLALIAEEVSFPKVIPKV
RKFLKETIEPWLDGTFKGNGFLYESKWRGLVTEQGSTDSTADFGFGIYNDHHFHLGYFLY
GIAVLAKIDPVWGQKYKSQAYSLVTDFMNLDQRYNSDYPRLRNFDLYKLHSWASGVTEFE
DGRNQESTSEAVNAYYAAGLMGLAYRDTDLVATGSTLLALEIRAAQTWWHVKVGDNLYEE
DFAKDNRIVGVLWANKRDSKLWWAPAECRECRLSIQVLPLLPVTETLFSDAVYAKELVEW
TLPSLKNKTNVEGWKGFTYALQGIYDKNTALKKIRMLKGFDDGNSFSNLLWWIHSR*
CcGBP1 protein2
>C. cajan_rna-KK1_019354_Cc_Asha_v1.0
SEQ ID NO: 83
MSSPFVFPETQSTVLPDPSTYFSPNLLSSPLPTSSFFQNFVIPNGSQPEYIHPYLIKTSN
TSLSASYPLLIFTAAVLYQAFVPDLTISSTQTQTKEQNRVVSSHSDLGVTLDIPSSNLRF
FLSRGSPFITASVTSPTSLSITTNHTIASLSSNDNKTKHTLRLNNTQTWLIYTSSPINLN
HDDGASEVTSKPFYGTIRLAVLPDSKYEATLDKFSSSYPLSGDATFENSKPFRLVYQWQK
KGSENLLMLAHPLHVKLLSKYNNAGVTVLHDFKYRSIDGDLVGVVGDSWVLEMDPIPVTW
YSNKGVNDGSRDEIVSALVKDVEALNSSAITTKSSYFYGKQVGRAARLALIAEEVSFSKV
VPTIKKFLKETIDPWLDGTFKGNGFLYEKKWGGLVTKLGSTDSTADFGFGVYNDHHFHLG
YFLYGIAVLAKIDPEWGQKYKPQAYSLVTDFMNLDQKYSTIYPRLRCFDLYKLHSWASGV
TEFEDGRNQESTSEAVNAYYSAALVGLAYDDSSLVATGSTLVALEILAAQTWWHVKVGEN
LYQEEFAQDNRIVGILWANKRDSKLWWATAECRECRLGIQVLPLLPITETLFSDAVYVKE
LVEWTMPYLSNEGWKGMTYALQGIYDKETALDEIRKLKGFDDGNSYTNLLWWIHSR*
PIGBP1 protein1
>P. lunatus_PI08G0000035500.v1
SEQ ID NO: 84
MSSSFLFPQTQSTVIPDPSTYFSPNLLSSPLPTNSFFQNFVIPNGTLPEYFHPYHIQSSN
SSLSASYPFLFFTAAVLYQVFVPDLTISASQTYSHGQNRVISSYSDLGVTLDIPTSNLRF
FLVRGSPFITASVTKPTSLSITTVHTILSLSSYNDNTKFILQLNNTQTWLIYTSSPIYLN
HAASEVTSKPFSGIIRIAALPDSDPNNVATLDKFSSCYPVSGDAALKKPFRVEYKWQRKR
SGDLLMLAHPLHAKLLSHDCNVTVLHDFKYRSVDGDLVGVVGDSWVLETDPIPVTWHSKK
GINKESFGEIVSALNKDVKELNSSAITTQSSYFYGKLVGRAARLALIAEEVSYPKVIPKI
IKFLKETIEPWLDGTFKGNAFLYERKWRGLVTKQGSTDSTADFGFGVYNDHHFHLGYFVY
GIAVLAKIDPAWGKKYKPQAYSLVTDFMNLGQRYNSDYPRLRCFDLYKLHSWASGLTEFE
DGRNQESTSEAVNAYYAAALMGLAYGDSRLIDTGSTLLALEIRATQTWWHVKAEDNLYEE
EFAKDNRIVGILWANKRDSKLWWAPAECRECRLSIQVLPLLPVTETLFFDTVYAKELVEW
TLPSLKNKTNVEGWKGFTYALQGIYDKTTALKKIRMLTGFDDGNSFSNLLWWIHSR*
PIGBP1 protein2
>P. lunatus_PI08G0000035600.v1
SEQ ID NO: 85
MSSSSSFLFPQTQSTVLPDPSTYFSSNLLSSPLPTNSFFQNYVIPNGSQPEYIHPYLIKT
TNSSLSASYPFLLFTTAVLYQAFVPDLTISSTQTHSHQQNRVISSFSDLGVILDIPSSNL
RFFLSRGSPFITASVTSSTSLSITTLHTILSLSSNDDDNTRYTLKLNNSQTWLIYTSSPI
HLNHNASEVTSKPFSGIIRVAVLPNPNYETILDKYSSSYPLLGDATLEEPSRVVYQWQKE
GSGDLLMLAHPLHVKLLSNNNDGNVTLLSDFKYRSIDGDLVGVVGDSWILQTDRIPVTWY
SNNGVETNSYEEIVSALVKDVQALNSSAIGTNSSYFYGKRVGRAARLALIAEEVSFSKVV
PTVTDFLKGAIEPWLDGTFEGNGFLYENKWGGLVTKLGSTDSSADFGFGVYNDHHYHLGY
FLYGIAVLAKIDTEWGQKYKPQVYSLVTDFMNLGQRYNRIYPRLRCFDLYMLHSWAAGVT
EFEDGRNQESTSEAVNAYYSAALVGLAYGDSSLVATGSTLVALEILAAQTWWHVKVEDNL
YEEEFAKDNRIVGIVWANKRDSNLWWAGADCRECRLGIQVLPLLPITETLFSDSDYVKEL
VEWTLPSLSSEGWKGMTYALQGIYDKQTALQNIRTLKGFDDGNSYSNLLWWIHSR*
PIGBP1 protein3
>P. lunatus_PI04G0000054600.v1
SEQ ID NO: 86
MLKKLRRKVSTALRSGLKNGSKPYKNPSPPPLSPLSLPLPLPLEPVRTMSHTTKHSPFLF
PHANSSWPDPSNFFSPNLLSNPLPTNSFFQNFTLKNGDQPEYIHPYLIKSSNFSLSLSYP
SRFFNSSFTYQVFNPDLTISSSQKPHLSHFNHTISSHNDLSVTLDIPSSNLRFFLVRGSP
FLTLSVTQPTPLSITTIHAILSFSSSDSLTKHTFNLNNGQTWILYASSPIRLSHGLSEIN
SDAFSGIIRIALLPDSDSKHEAVLDRFSSCYPVSGEAVFARPFCVDYKWEKKGWGDLLML
AHPLHLQLLADGGCGDVNVLSDFKYGSIDGDLVGWVGDSWSLKTDPVSVTWHSIRGVREE
SRDEVVSALVNDVEGLNSSSITTNSSYFYGKLIARAARLALIAEEMCFLDVIPKVRKYLK
ETIEPWLEGTFNGNGFLYDRKWGGIVTKQGSNDAGADFGFGIYNDHHYHLGYFVYGIAVL
AKIDPVWGRKYKPQAYSLMADFMTLSRRSNSNYTRLRCFDLYKLHSWAGGLTEFADGRNQ
ESTSEAVNAYYSAALMGLAYGDTHLVATGSTLTALEIHAAQMWWHVKQGDNHYGEGFEKE
NKVVGVLWANKRDSGLWFAPPEWKECRLGIQLLPLLPISEVLFSDVDFVKDLVEWTLPAL
NREGVGEGWKGFVYALQGIYDNEGALQRVRSLNGFDDGNTLTNLLWWIHSRSDEEEFGHG
KHCWFGHYCH*
PIGBP1 protein4
>P. lunatus_PI04G0000054700.v1
SEQ ID NO: 87
MFKKLGRKIEREITKPFKNKPRPRTSSPPPPPPPPPPPPPPPPPPPPPSPPPLPKQPNAP
FLFPQAHSTILPDPSTFFAPNLLPSPLPTNSFFQNYVLQNGDTPEYIHPYLIKSSNSSLS
LSYPSLNFNSSFIAQVFNPDITISSTDSKTTPGLHASHVISSFSDLSVTLDIPSSNLRFF
LVRGSPFVTASVTCPTPLSITTMHAILSLSSNNSLTKHTLQLNNGQSWLINTSSPISLNH
SLSEITSGEFSGIIRIAVLPDSDPKYEVILNRFSSCYPVSGDATFTNPFCVKYKWEKKGW
GELLMLAHPLHLQLLNDGDSGVTVLHNLKFRSIDGELVGVVGDSWLLKTDPVSVTWHSTR
GIKEEFHEEIYSVLSEDVEALNPKGITTTSCYFYGKIIARAARLALIAEEVAFLDAMPVI
RKFLKEIIEPWLDGTFSGNGFLYEGKWGGIVTKQGSKDSEADFGFGVYNDHHYNLGYFLY
GIAVLAKIDPAWGRKYKPQAYSLVADFMSLARRSDSNYTRLRCFDLYKLHSWAGGLTEFA
DGRNQESTSEAVNAYYSAALTGLAYGDTQLIATGSTLAALEIHAAQMWWHLGEGNKLYEE
DFTKDNKVVSVLWANKRDSGLWFAPSQWRECRLGIHVLPLSPITEALFSDVDYVKELVEW
TVPNLNRKCVGEGWKGFIYALEGTYDKESALQKVRSLKVFDDGNSMSNLLWWIHSRGDVE
EEFGQGKQCWFGHYCH*
PIGBP1 protein5
>P. lunatus_PI04G0000054500.v1
SEQ ID NO: 88
MVKQNKTHFIFPETQSTVLPDPSNFFSSTLLSKPLPTNSFFQNFVLKNGDQPEYIHPYLI
KSSNSSLSLSYPSRQVSSAVIYQVFNADLTISSKQSSSGKHLISSYSDLSVTLDIPSSNL
SFLLVRGSPFLTVSVTQPTPLSITTIHTILSFSSNETNTKYTFQFNNGQTWILYASSSIK
LSHTLSEITSDTFSGIVRIALLPDSDSKHEAVLDKFSSCYPVSGEAIFREPFCVEYKWEK
KGSGDLLLLAHPLHVQLLSNGDNDVTVLEDFKYGSIDGDVVGVVGDSWVLQTDPVYVTWH
STKGVKEESHDEIVSALSNDVDGLNSSSISTTSSYFYGKLIARAARLALIAEELSYPDVI
PKVKKFLKETIEPWLVGTFNGNGFLHDKKWGGIITQQGSNDGGGDFGFGIYNDHHYHLGY
FLYAIAVLVKLDPAWGRKYKAQAYSIVQDFMNLDTKLNSNYTRLRCFDLYVLHSWAGGLT
EFSDGRNQESTSEAVCAYYSAALMGLAYGDAHLVSLGSTLTALEILGTKMWWHVEEEGKL
YEEEFTRENRIMGVLWSNKRDTGLWFAPAEWKECRLGIQLLPLVPISEAIFSNAEYVKQL
VEWTLPALNRDGVGEGWKGFVYALEGIYDNESALQKIRNLTGFDGGNSLSNLLWWIHSIG
NE*
PaGBP1 protein1
>P. acutifolius_Phacu.WLD.008G033800
SEQ ID NO: 89
MSSSFLFPQTQSTVLPDPSTYFSPNLLSSPLPTNSFFQNFVIPNGTVPEYFHPYHIQSSN
SSLSASYPFLFFTAAVLYQVFVPDLTISASQTYSNAQNRVISSYSDLGVTLDIPTSNLRF
FLVRGSPFITASVTKPTSLSITTVHTILSLSSYDDNTKFILQLNNTQTWLIYTSSPIYLN
HAASQVTSKPFSGIIRIAALPDSNPNNVATLDKFSSCYPVSGDAALKKPFRVEYKWQRKR
SGDLLMLAHPLHAKLLAHDCNVTVLHDFKYRSVDGDLVGVVGDSWVLETDPIPVTWHSKK
GIDKESFGEIVSALNKDVKELNSSAITTQSSYFYGKLVGRAARLALIAEEVSYPKVIPKI
TKFLKETIEPWLDGTFKGNAFLYERKWRGLVTKQGSTDSTADFGFGVYNDHHFHLGYFIY
GIAVLAKIDPAWGKQYKPQAYSLVTDFMNLGQRYNSDYPRLRCFDLYKLHSWASGLTEFE
DGRNQESTSEAVNAYYAAALMGLAYGDSRLVDTGSTLLALEIRATQTWWHVKVEDNLYEE
EFAKDNRIVGILWANKRDSKLWWAPAECRECRLSIQVLPLLPVTETLFFDSVYAKELVEW
TLPSLKNKTNVEGWKGFTYALQGIYDKTTALKKIRMLTGFDDGNSFSNLLWWIHSR*
PaGBP1 protein2
>P. acutifolius_Phacu.WLD.008G033900_1
SEQ ID NO: 90
MSFSSSFLFPKTQSIVLPDPSTYFSSNLVSSPLPTNSFFQNFVLLNGSQPEYIHPYLIQT
SKSSLSASYPLLFFTAAVLYQTFVPDLTISSTQTLSNEQNHVISSHSDLGVTLDIPSSNL
RFFLSRGSPFITASVTSSTSLSITTLHTILSFSSNNENNTKYTLKLNNTQTWLIYTSSPI
HFNHNASEVTSKPFSGIIRVAVLPNPNYETILDKYSSCYPLLGDATLEEPSRVVYQWQTE
GSGDLLMLAHPLHVKLLSNNNTGTVTILHDFKYSSIDGDLVGVVGDSWKLEMNHIPVTWH
SNKGVEKESYDEIVSALSKDVQALNSTPIATASSYLYGKLIGRAARLALIAEEVSFPNVV
PTIKEFLKENIEPWLDGTFQGNGFLYENKWGGLVTKLGSTDSSADFGFGVYNDHHYHLGY
FLYGIAVLAKIDLEWGQKYKPQVYSLLSDFMNLDHQHNAYYPRLRCFDLYMLHSWASGLK
EFADGRNQESTSEAVNAYYSAALVGLAYGDSSLVATGSTLVALEILAAQTWWHVKVGEKL
YKEDFAKDNRIVGVLWANKRDSGLWWASAECRECRLGIQVLPLLPITETLFSDADYVKEL
VEWTLPSLSSEGWKGMTYALQGVYDKQTALQNIRTLKGFDDGNSYSNLLWWIHSR*
PaGBP1 protein3
>P. acutifolius_Phacu.WLD.008G033900_2
SEQ ID NO: 91
MSSSSSFLFPQTQSTVLPDPSTYFSSNLLSSPLPTNSFFQNYVIPNGSQPEYIHPYLIKT
TNSSLSASYPLLLFTTALLYQAFVPDLTISSTQTHSHQQNRVISSFSDLGVTLDIPSSNL
RFFLSRGSPFITASVSSSTSLSITTLHTILSLSSNNDNNTKYTLKLNNTQTWLIYTSSPI
HFNHNASEVTSKPFSGIIRVAVLPNPNYETILDKHSSCYPLLGDATLEEPSRVVYQWQKE
GSGDLLMLAHPLHVKLLSNNNNGNVTLLSDFKYRSIDGDLVGVVGDSWILQTDRIPVTWY
SNNGVEKNSYDEIVSALVKDVQALNSSAIGTSSSYFYGKRVGRAARLALIAEEVSFSQVV
PTVTDFLKKAIEPWLDGTFEGNGFLYENKWGGLVTKLGSTDSSADFGFGVYNDHHYHLGY
FLYGIAVLAKIDPEWGQKYKPQVYSLVTDFMNLGQRYNRNYPRLRCFDLYMLHSWAAGVT
EFEDGRNQESTSEAVNAYYSAALVGLAYGDSSLVATGSTLVALEILAAQTWWHVKVEDNL
YEEEFAKDNRIVGIVWANKRDSKLWWAGADCRECRLGIQVLPLLPITETLFSDSDYVKEL
VEWTFPSLSNEGWKGMTYALQGVYDKQTALQNIRTLKGFDDGNSYSNLLWWIHSR*
PaGBP1 protein4
>P. acutifolius_Phacu.WLD.004G045300
SEQ ID NO: 92
MFKKLGRKIEREITKPFKNKPRPRPSSPPPPPPPPPPPLPSSTPPPPPPPPSPPPPLPKQ
PNAPFLFPQAHSTILPDPSTFFAPNLLSSPLPTNSFFQNYVLQNGDTPEYIHPYLIKSSN
SSLSLSYPSLNFNSSFIAQVFNPDITISSTESKTTPGLHARHVISSFSDLSLTLDIPSSN
LRFFLVRGSPFVTASVTCPTPLSITTMHAILSLSSNNSLTKHTLQLNNGQSWLINTSSPI
SLNYSLSEITSGEFSGIIRIAVLPDSDPKYEVILNRFSSCYPVSGDATFTNPFCVKYKWE
KKGWGELLMLAHPLHLQLLNDGGDSGVTVLHNLKFRSIDGELVGVVGDSWLLKTDPVSVT
WHSTRGIKEEFHEEIFSVLSEDVEALNPLGITTTACYFYGKIIARAARLALIAEEVAFLD
AMPVVRKFLKEIIEPWLDGTFSGNGFLYEGKWGGIVTKQGSKDSGADFGFGVYNDHHYNL
GYFLYGIAVLAKIDPAWGRKYKPQAYSLVADFMSLGRRSDSKYTRLRCFDLYKLHSWAGG
LTEFADGRNQESTSEAVNAYYSAALMGLAYGDTQLIASGSTLAALEIHAAQMWWHLGEGH
KLYEEDFTKENKVVSVVWANKRDSGLWFAPSQWRECRLGIHVLPLSPITEALFSDVGYVK
ELVEWTVPNLNRKCVGEGWKGFIYALEGTYDKESAVQKVRSLKVFDDGNSMSNLLWWIHS
RGDVEEEFGQGKQCWFGHYCH*
PaGBP1 protein5
>P. acutifolius_Phacu.WLD.004G045200
SEQ ID NO: 93
MLKKLRRKVSTALRSGLKNGSKPYKNPSPPPSSPLPLPLVPVRTMSHTRKHSPFLFPHVD
SSVVPDPSNFFSPNLLSNPLPTNSFFQNFTLKNGDQPEYFHPYLVKSSNFSLSLSYPSRS
FNSSFTYQVFNPDLTISSSQKPHLSHFNHTISSHNDLSVTLDIPSSNLRFFLVRGSPFLT
LSVTQPTPLSITTIHAILSFSSSDSLTKHTFNLNNGQTWILYASSPIRLSHGLSEINCDA
FSGIIRIALLPDSDSKHEAVLDRESSCYPVSGEAVFARPFCVEYKWEKKGWGDLLMLAHP
LHLQLLADGGCDVNVLSDFKYGSIDGDLVGVVGDSWSLKTDPVSVTWHSIRGVREESRDE
VVSALVNDVERLNSSSITTNSSYFYGKLIARAARLALIAEEMCFLDVIPKVRKYLKETIE
PWLEGTFNGNGFLYDRKWGGIVTKQGSNDAGADFGFGIYNDHHYHLGYFVYGIAVLAKID
PVWGRKYKPQAYSLMADFMTLSRRSNSNYTRLRCFDLYKLHSWAGGLTEFADGRNQESTS
EAVNAYYSAALMGLAYGDTHLVATGSTLTALEIHAAQMWWHVKQGDNHYGEEFERENKVV
GVLWANKRDSGLWFAPPEWKECRLGIQLLPLLPISEVLFSDVDFVKDLVEWTLPALNREG
VGEGWKGFVYALQGIYDNEAEVMKRNLVMENTAGLVITATRQLLPDECPL*
PaGBP1 protein6
>P. acutifolius_Phacu.WLD.004G045100
SEQ ID NO: 94
MVKQNKTHFIFPETQSTVLPDPSNFFSSTLLSKPLPTNSFFQNFVLKNGDQPEYIHPYLI
KSSNSSLSLSYPSRQVSSAVIFQVFNADLTISSKQGSSGKHVISSYSDLSVTLDIPSSNL
SFLLVRGSPFLTVSVTQPTPLSITTIHAILSFSSNKTNTKYTFHFNNGQTWILYSSSTIK
LSHTLSEITSDAFSGIVRIALLPDSDSKHEAVLDKFSSCYPVSGEAIFREPFCVEYKWEK
KGSGDLLLLAHPLHVQLLSNGDNDVTVLEDFKYGSIDGDVVGVVGDSWVLQTDPVYVTWH
STKGVKEESHDEIVSALSNDVEGLNSSSISTTSSYFYGKLIARAARLALIAEELSYPDVI
PKVKKFLKESIEPWLEGTFNGNGFLHDKKWGGIITQQGSNDGGGDFGFGIYNDHHYHLGY
FLYAIAVLVKLDPAWGRKYKAQAYSIVQDFMNLDTKLNNNYTRLRCFDLYVLHSWAGGLT
EFSDGRNQESTSEAVCAYYSAALVGLAYGDAHLVSLGSTLTALEILGTKMWWHVEEEGSL
YEEEFTRENRIMGVLWSNKRDTGLWFAPAEWKECRLGIQLLPLVPISEAIFSNAEYVKQL
VEWTLPALNRDGVGEGWKGFVYALEGIYDNESALQKIRNLAGFDGGNSLSNLLWWIHSIG
NE*
CaGBP1 protein1
>C. arietinum_NC_021161.1
SEQ ID NO: 95
MSSSSVPFLFPQTHSTILPNPSNFFSPNLLSTPLPTNSFFQNFVLQNGDQPEYIHPYLIK
SSNSSLSFSYPLLLFTTSFLYQVFVPDLTISSSQKTTSKNKHVISSYSHLSVTLEIPSSN
LRFFLVRGSPFITANVTKPTSLSITTLNKIVSFSSFDYKKTKHTLQLNNTQKWIIYTSSP
INFNHDGFEVISNPFSGIIRIAIVPNSNPFYEKTLDKFSSSYPVSGDANIKKNFSLVYNF
QKKRLGDLLMLAHPLHVKLLSNDVKVLHDFKYKSVDGDLVGVVGDSWLLKNDPVSVNWYS
NKGVAKESHNEIVSALIKDVNELNLSSISTTSSYFYGKIVGRAARFALIAEEVSYLKVIP
KIKFFLKETIEPWLNGNFKGNGFLYEKKWGGLVTQQGLNDSSADFGFGVYNDHHYHLGYF
LYGISVLVKIDPLWGQKYKPQVYSLLKDFMNLGERDNKNYPSLRCFDHYKLHSWASGLTE
FENGRNQESSSEAVNAYYSAALIGLAYGDSKIVEIGSTILAFEIKAAQTWWHVKLENNLY
GEDFAKENRIVGILWANKRDSKLWWAPSECRECRLSIQVLPLLPISETLFFDGVYAKELV
EWTLPSLKNKTNVEGWKGFTYALEGIYDKEIALKNIRGLKGFDDGNSFTNLLWWIHSR*
CaGBP1 protein2
>C. arietinum_NW_004515975.1_1
SEQ ID NO: 96
MSTIKKNTPFIFPQTNSTVLPDPSNFFSPNLLSTPLPTNSFFQNFSLKNGDQPEYIHPYL
IKSSNSSLSVSYPSHFSNSSFIYQVFNADLTITSLEQKTNQTSNEKHIISSYSDLSVTLD
IPSSNLSFFLVRGSPYLTFSVTKPTPLSISTIHAIEFLVPTDPSITRYTFQLNNGQTWLL
YASSPIKLSHDLSEITCEPFSGVIRIALLPNNDRKIEDVLEKYSSCYPLSGDAFLREPFC
VEYKWQKNGSSDLLLLAHPLHVKLLSNSESDVTFLNDLKYTSIDGDLVGVVGDSWILKTE
PVSITWHSSKGVKEESHDEIVSSLSKDVEGLNSSAITTTSSYFYGKLIARAARLALIAEE
VFFFDAIQKVRNFLKETIEPWLEGTFNGNGFLYDRKWGGIITQQGSNDSNGDFGFGIYND
HHYHLGYFLYAIAVLVKIDPTWGRKYKTQAYSLMEDFMNLNIRLNSNYTRLRCFDLYKLH
SWAGGLTEFSDGRNQESTSEAVNAYYAAALMGMAYGDASLVSIGSTLTSLEILGTKMWWH
VKKEGKLYEEEFTKENRIMGVLWSNKRDSGLWFAAAESREARLGIQLIPLSPISEVLFSD
VSYVKDLVEWTLPALNREGVGEGWKGFLYSLQGVYDNQGALEKIRNLNGFDGGNSLTNLL
WWIHSRGEDGDDE*
MtGBP1 promoter
>prMtGBP1
SEQ ID NO: 97
GAAGGAGATTTATAGGGGGAGAGAGTGAGGAGGAAAAAATGTGAGATGGTGTGAGGAAAA
AATGTGAGATGTGGTTTTGGTTTCAAAATGCTCGTGAAGCTGAAATTCCTAGCTTCTATG
ATAGGGAAAACATGATTCAACCTTTAATTTCTCAAAGTGGTAGGTACTTTACCTAATTAC
CTATAAATTTAAGCCTTCAATTTCTCTTTCTCACAATTTTTCTTTGTTTCGTTACTTCAC
ATCACTTCAACCTTAAATTTTCTTGTGAAGATTTTGCATATCCAAGCTTTCGATTTCCTT
CAGCTTTTCCCTTATTTTTGCAAATTTCGTGGTAATCCCATTACTCATAGTAGATGTGTT
AGTTGGGTGATATTATTGCATATTACTCTATATTTATATATGATGCTTATGAGCTACTTG
TGAGAAACAATCCATTACTCTGCCAAAGTCTGAAGGTGAATACATATTCATATATGTTTT
TCTTTGCTTTGATTACTTGTTTCAATATTGTTAATTTCTATTTCTATTTGCTCTAATTGT
TACAAGTTTTACCTTCATTTTTTCATCCTACATCAATGCATTGTACGTGATTGGAACCAA
TAAAATCCATGTATGATAAGTTGAAATAATTATCTAATGAGAATGAAATTTATTTTTAAT
ACAATTAAAAGGATAGTTTGGTCATTACACATTAAAATTAATTTTGATTTAAACTATCCA
AACAACATCAATTCATAAGATTCAATTATATTAAAGTGTATCCAAACATAAATCAATTCA
CATCCAACTCACTTTTAACCAAAATCAATTCTCTCAAACTCAATTTTATTAAAATCTATT
CTCTCAAACTTAATTTTAACCAAATACACACTTAATAGAACAAAAAAATATAAAAGATGA
ATTGGCATGAATTTTAAGAGCATGCATGAATTCGCGTGACAAAAACATGAGGAAACATTT
CTTAAAATAAGGAAACATTTTTTTGACCTATAAACTACAACTACTCCAACTCAAACTCAC
GAGAATGGTTAAAACACCACCCAAATTTCCACCCAAAATATATTTTACCCAATTAATAAT
TTACTGGCGATACAATGAACAAAGTAAGATTAATTACAATCTTATTTTTTTGACAAAGAT
TAATTACTATCTATTTATGTGATTTGAAATTATGAAGCCCGGATACGGATACGATACGGA
TACATGATTTTTTTTTTAAATTTAGGATACGATACAACTAGGATACATTAACAAATATTT
TATATATGTATGTAAAATACTTGAAGAAAATAATCATTGATTATTCTTCATTATTATATT
ATTATAAAAGTCACAATTTTAAGAAAAAATACATGTAATCAAAATAAAAAAATGAAAAGT
TTTTTTATCAACCATATTATATTTTCATATATGTTAGGAAATTGAGAGAAGCATCATTAA
AAACACTCAACATAAATTTTATTTAATGTTGATTAACTGAATCACCTCTCTCGTTACCTT
CATTCACAAGAAGTATAATTTTAAGTGATTCATCGAGAAACAATTTTATTGATGCATATC
TAAAATTGAATCATATACATCTCTACTAAGTTTTCACAACATTGTCTCACTTTTTTTACC
CTGCATCTTTTTTAAGATATGTATATGTGAAGTATCTTAAAAGTATTTTTGTGTATCTCT
GCAGTAACATATAAGTATCTGATAAAATAATTTTTAACAATTGAAATAATTAACTCCGAT
ACGTCGTGGGTACGTATCCACGAATATCCGTGGAGTATCGATATCCGATACGGGTACGTC
TCCGTTTTGGAGTATGCGAGGCTTGATAGAAAATTTATATCTTACCATTAAAATTTGTAA
CCAAGCAAGCAAATTAGTTGATTGGACCCCATTGTTTCTTCGTGCATGATTGATATTTAT
TTGTTTATTTCAAAATAGTAAAAATCATAATTTTGACTAACATACAGTGGCATCAAATAA
TAGACAGTTATCAATTGTTTTTTATTGCTGACAGAAAACCTTTTGCTACAATAATAAACA
ACATTTACACAATCATTCTCTAAGCATTTGAAAGCACAGTTACATATAGATTTGACAAAC
ACAATCATTCTCTAGCTATAGTTTCAACAATCTTTTGGTCATCTGTGATAGATAAGAATT
TAGATTATGGAAGGGACACCAATGTAAGTATTTTATCTTTCGGTCCTCTAGTAATTGTTG
AACGGAATAGTTAACCTTAAAACATCTAAGTGCCAAAACATCCACACATATATTAACCCC
TAAGATCATTTCCAATGGAGGAAGCTTTTCTACATATGTTATGACATCTAATAGGTACTT
CTATAATAGAAAAATAAAAATTGTGTTAATGAGAAGAGTGACTTTGTTAAGCTCTCTTTA
TTTTAATGTTAAAATATTGAAATATAATGATATTAAACTATTTTTATATGATATAAAATT
GTCTATAAGTCTGTACAAGAAATTTGTGTGACGTGCAAAGTTAGAGATATTGAATGTTTA
TAAAGTTATATATGAGTGATACGTAGTTCTGAATGCCGATTTAATGTCAAAATAAGTAGA
CTTTTTTTGAAGGAGAATAAGTAGCCCTTATCATTTCACCTAAAAAAATATTCTTGTGAA
TATCTTTTTTAAGAACCTTTATTTTAAAGTAAGGTTTATATGCTCAACAATGTCACCTTA
GCTCTTATATAAACAAGGAAAGGAAAACAAGCACTAACCAACACAAACATTGTAACAACA
TCACTCTCATCCACCAAAACACAATAACA
PsGBP1 promoter1
>prPsat3g201680
SEQ ID NO: 98
TATTATGGGACATCTTGGTGCGAAGATTGCTAGTATTGCATTTAGTTAGAAAATCTTTAT
TATTGTGGAACGATAAGAAGATGCACTTTGTTAATTCAAACTAGTTTGGAAAAAAGATTT
GGATTTAGAATATTTGGAAGATTGGATTTACAATGGAGAAAAGATTTAATTGGATATGCT
TTGAATGAGGACTTGGTTCTCTATTCTAAGTTGTTTGGTAGTATTCCTCTGGTTTTCTTT
CTTTTCACGTCTTTGCCAAATTATGGCAAAAGGGGGGAGAAATGGTGTGAGCTCATGATT
GATGCTAGACTAACTCATGTATGATGTTGATGCAGTTCTGATGCGGTTTATGGAGTGTTA
TTTTTTGGAGTTTTGATGCAGTTCTGATGAGGAGCATTATTTAACATATGTGTGCGCACA
TGCTGAGACATCTGGCCCTAAAATCCGTGTGACTGTGTGTGCTGATTTCTTTATGTGCTA
GTAGTAGCTTCTGAGTCATGTCTACTGACCATTAAATGTGTGTGCATGCATGACTAACTG
ATCTTGAATGATTGAACGACTGACTATTATAGAGCGTTAATGTGTAGCAACTGCTTTGCT
ACTAATATGTGTTACTACTGCTCTGACTTCTGCTACTGATGTATGCTCTACATTGATGTT
CTTCTGCTGCTGGTGATGTTTCTTATGAGTCTGGGATTCCCTTGATACTCAGTTGTGTTT
CTGCTTGAATAGTTATTTTGCCAAAAAAAATTAAAGGGGGAGATTGTTGTTCCTTTAGGA
TTGACTGCATTATGCAAAATAACATGGAGTATGTTCATTGTGTTTCTGGTGAAGAAGGGA
CACACGTGGAACATGATGTTCCAACATGTGTGTGTTAGCCTAGAATTTTCACTTAGTAGA
AAAGTCAATTGAATGTTGCATTGGGAAAATGTTAAGCATTAGCATTGATTGAGATACGTC
GACAGTGATGGTATGTCGACTAGAGTGTTTTGACTGGCATCTTGAGATGTGGTTTGGAAG
AATCTTAAGAGAAGATCATGAGATATGGTTTGAAGGAGTTTCATGAATAAATATGGGTAA
TCACTCGACGCCATGTCGAGGTCGATACCTTCGACGGGTTCCAAAGACTTAGAATATTGA
GAAAACCATAGAAAGAGGAAGGTACTTAGCGCTATCATCAACTACATTCGACGAGCAGTA
AGATACTAGTCGCTAAGAGTAGTTAGCGAACATTGGGTAATGGCTTTCAAATTTTTTCAA
GTATTGACGACGTGGCAATGCATCAGAAGAATATGTTGTATGTCTTAGAAGACATGTCGA
TTATTTAGGAGTGAACTGTAGGGAACTGTTATTTTTAGTTTAGTATATATAGTTGGCTTA
TATTTTGTAATAAAGGTCACAAATTTTAGATACTTTATATATAGAACTGAAGTACCCAAG
CCAAAGTGTGAAACATTTTGTGAGTGATATGTGAGTCTGCATCCCTTTCATTGTATTTTC
TTTATGTTTTCTTAATTGAAACATCTTTTCTTTTCTTCATTTACTGCATTTATTTAATGT
TTTTATTGTAAATTTCTTTTAGTTTAACTGAAGCTTTATTGTCTTTAAAGTTTATTTTTA
TCTTGTTTGCATACAGACACTCATAATCACTTCATATACATATTTTCTTGTAAACGTGTT
TGCCAATAATTACTTCAGAGATTTTTCAATTTAATCTCACAAACTTTCAAACTCGTGATT
GTTCTTTAAAAACACTTGCGAATAGTGTGATACAACATCTGGTCTCTGACAATAGACATG
TGTTATGGAAGTTCTAGTTCATATTTAATCCAAGATTTACTCTACAAAGAAGATTTATAT
AAAGAGATTTAATTTGGATGTTCAGGTGAAACATATGGAACACGACGTTCCAGCATGCGT
GTAGTGCAACATTTGGCCTTGGTCAGCAAGCCATGATGAATGTTGTTAGGTGCAAAATTC
TGATTGAGATTCAATTAGATCTTTTGCAGATTGATTTGTAATGTTTGTTTGATTTGTTTG
AAATCTATTGAAGATCAAATTAGATTTAAAGGAGATTTGAATTTGAATTTGAAATATAAC
AAATCATATATTTTGTTGGAGCGATTTATGGAGAAAATCAAATCCAATTGATTCAACTAT
TATTCTGGATGAAATTAAATTAGAAATCCAAAAGAGAAAAGACCATAATGGATTGCTATT
TAGGGAGACTCAAATCTCATGTTTGAGAACAAGTTTTGTGCCCTAGGAGTAAGTTCTTTT
TGCTAGTGTTTTTGATTATTTTGTTATTTGTATTCCACAATAATGTTATTAGTCATATCA
TAAGGATTAGTGTTAGATCTTGGTTGTGTGCACCACACTATTGGTTTAGTCACTTGACAT
AGTTGTTGATTTGTCTTAGTTGAATTTTAAATTCATCATTCAATAGAGTTATTATTGAAG
TTGATCACGTGGGATTAATGATTAAGAGAAAGTGAGAGGGATTCTCATATTTAAGGGGAG
TCATAAATAGAAAGTCACTATGATTAGGAAGAGGCTTTGAACAACACACAGTTGATGTTC
CTATAAGACTAATTGTACTAATTCAAGATAGGGAATTTCCTTTCTTTGGTATAGTGCCCT
TTTGATGTAGGTGTTGTTTGCACCGAAATGAGTTACCAATTCTCTTTTGTTATTTTATTG
CTTTATGCATTGTGATATTGTTATATTATTGTCATACTGGTTTAACAAGTTGGACCAGTT
GTCCCAACATCGTGTCCAACATCTATCATCTCAGGAACAAAATTTCAGCTTCCAATTTTC
TTGTTTAGAGGGAGAAAATAACTACTTCTATGTCATCTCTTTCATTTCAGAATCATATTG
TTTTCCTTATTTGGATGAGTTGAGCTTCATCTAAGATATTGGGCTTTTCTTGGCAGTTAT
TGCAGGATCGATACCTTTCGAGAGTAAACTTGTTTAGAAGAGAGGTGTTATCAGATTCTA
GTAGGCTTTCTTGTCCTTTTTGTGGGTTTTCGTCTGAGACTGCTTCTCACCTTTTTATTA
CTTGTGAGACAGTCTTGTCAGTGTCGTATAGAGTTTTTCAGGTAGTTGGGGTGCCAAGTA
GCTATTCACCACGATCTTAGGTTGCGCTTTGAGTACTTTCATTTTTAGGAAGTAGGGTTA
AGTATATGAGCGATTATCTTATGGTTTGGCATCATGATGTAATCTGGTGTATTTGGAGAG
TCGGAAATGATGTTATATTTAATGGAGTCGCAAAAAGAGAGAAAGAAGTGGAGGAAAATA
GTTTTTTTTTGGCATGGAATTATTTTTTGGGTCGGACGGGGGATAACTCTTGCACCTTCA
CATATTGGCTTTAAAATCCTATCCTCTCCCTACACCTATAGAGGATCGAGTGTTTACACT
AATTCTGAGTGTGGTGGTGGATCTTTTTTTTCCCATCTTTTAGTGTGATTTGATTTGTTG
ATGGTGAGTCTAGTTGATTTGGAGATCTAACCTAAGAGTAGCGACGTGATTCTGAAGAAA
TTGTGGTTTTGATTCTGGATGTCCCTCGGTTCAAATCTTCTAGATTTTTTCTATTTTTTG
TTGTTTTTTATTTTGTTTTCAAACACTTCTTATGGTTTGTATTTTCTTTTTAGATTTTTG
TATCTTGAGATTTTGTCATAACTAATATATTTATTTTGCCATTAAAAAATAGTGATAAAA
ATACTCTAAATACTTGTATTTTTGAAACCAATAAAAAAATATTCAAAAGAGTAATTACCA
CGAAGAATTTTATCAAAATTCAAATATGCTTCTTCAAACAAACATAACATATATCTAAAA
AAATTTAATTACCATTACTAAGACTTTATAAACAATTTATTTTAAAGTCAACTTTTACAT
TCTCAACAATCTCACCTAATTTATAATTTCATTAACTATAAAAACCATACCAAACCAAAC
AAAATTAAAAACAAATCCCATCAAAAACATAAACCACCAA
PsGBP1 promoter2
>prPsat3g201640
SEQ ID NO: 99
GATGTATAAAATTTTTTGTACATCAGAGAACACACCAAAAATCTGAACCTCTTTCAAGTC
ATATATTTCAATCCATTCATCTATTGGTTTTTAATGTTTTCATAACATTTTGACATAAAC
CTTTTTGTTCATAAATTTCAATCATTAAGAAAAATATTCTAACCTCTTTAGCATTTATTG
TTTGTATATTTCTTGAATTGAATCGTTGAAATAGAATATCAAAAGTATCAAGATATTCAA
AGATCATTCTAACTTGTGTGTAAATTATCAATTGTCTAAATCTGTACTTTTTCATTGAGT
GTGGTAATATTCTTTGTTTGTAAGAACGTGGTTTGTACTTTCTATAGGTGTGCTCAAGTA
GGAAGTTTTGTTCTTTATTTTTGTTGGAAAGTATGATATATTTTTCTAACAACCTTTCAT
GAACGTTGACCGATTAAGGGAAATCACGCTACCAATCTTTTTACTTAATCGGTTTTCTAA
CACTTTCGTCGTGTGAAAAAGATGTTTGTCATCATCTTATAACATGCTTTGATGAAGACA
AAATGTATTATCTTATAAGAAATATTAAAAGAGAAAAAGTTTGAAATAAAGTTTTCAAGA
TTGTTTAAGCTTTAATAATCAATGGATTAAAGATGAAGGTTTAAATACAATGGTAAATCA
TATGTATATAAGTATTAGGTGCTCAGAACTATCTTCATCTAAAATCTCTAGCATGTGCAT
ATACTTTCACACAAGTTTAAAAGTTTTCAAACTTTCATTTTTGTAAATTGTTGAGGTTGA
ATTCAACTATAACAAAGTGTAACCGAATTCAGATGGCAAAAAGAGATGAAATTAAGCAAT
TTGTATTCAACTATAAAGATATTTATTTGAATACAAGTTCTGTTGTATTTAGATACTAAG
ATGTTTGTTTGAATTCAAGACTTAAAAGTTCGAATTTTCAAAATGTAGAAAGGACTATAT
TAGACTACACACATCCTGTAGCCGAATAAACATTCAAATCAGTGCATTTTTTCACTACTG
AACCTATTTCAGAGTAGTTCAAAGTTTCATGATTGTTTGTTAATGATTGTATCTTTATAA
ATAGGTGTTTTAGGATGTATAAAAGTTTTTGTACATCAGAGAACATACCAAGAATCTGAA
CCACTTTCAAATCATATTTTTCAATCAATTTATCTACTATTTTTCAATGTGTTGGCCCAA
ACACAAAATTAATTAGGATTTAGGGTTTGTTATATTTGTCACTTAAGGGTTAATTCACTT
AATTGTATATAAAGAAATACTTTGTAATTCAGTTTTGTAACACAGTCCTAATATTTAATA
ATAATAATCGTTATTTCTTCGTTCTCTCTTTTCCTCTCCTCACTAAAAGTACCTCACGCA
CAAGCAACTCATCCCCATAAAATTGACTTATAGGGTAAGGGGTGTCACTATATATAAATC
ATTTGTGGCCACTATCTTTAACCAATGTAGGACTTGGGTTTTTTCGAATACACCCCCTTA
CATCCAACACTATCGGTCTTGGTGCGTGGATATAAATGGTGGGTGACCCGAATTATCGAT
ATGCAAGACTTTGTTTTTCCAATACACCCCCTTACGCCTAATATTTTTTGGGTTGGGTGT
GCAATATATAAAGTGTGGATGACCCGAATCGATAAACTCGTGATACCATGATAAAATTAG
AGTTTGAACTAACTCATCCTTACAAAATCGACTTAGAGTGTGAGGGGTGTCACTATATAT
AAATCATTTGTGGATAATATTTTTAATCAATATAGAATTTGGGTTTTTTCAAATACTAAA
TAACTATATTTAAATTTTTATAATATTTCTTATAAAATTGTTTTATAAAATATTATAATA
ATATCAAGATATCTAATACGATATCTAAAAAACCATGTTTAAAAAGAATGACATAAAAAC
TAAATAAAAAATGTTTTAAAGAGACTCTAAATGCTTGTATTTTTTAAACAAATAAAAATA
TTCAAGAGCGTAGTTTTAGTGAAACTTATTTTTAGAAACAAGGGTAATGCTAACTTGGCT
CTAGGGGCACAAGATAAGACACTCACTTATAGAAAGTTTGTATTGAAAAAATCAATTATA
AAATTAATTTTATATCTTTATTAAAATCAATACACAATTTTCAACACAAAACTAATTTCT
TTAGTTCTTATCTTGTGCTCCTAGGGCACAAGTTAGCATTATCCTAGAAACAATAAAAAG
AAAAATATCTCTAAATAATTACTACCTCCTTCTTTTATTTTTCCTATTATACCTTTAAAT
ATTTATTATTCTCACTCCATTCAATTATCTCAATTTGTTTTTTCAATACCATTAATGAAG
CATAATTTTGTAAAATTCTTCATAATTTATTTTTGTCATACCATAATTATTACATTTCTT
AATACGTGTGAAAAGTCAAAAACGACTTATAATAAAAAACGGAGGGAGTATATTTTAATT
TCTATAATAAATCTTTTTATATTTTGTTGCAAAATATTATATTAACAGAAATATATCTGA
TACCATCCGGAAAAAATTCTATTTAAATAGAATGACATAAAAACTAAACAAAAAAAATAT
GTTTTAAACATAATCTAAATGGCTGTAGTTCCAAAACCAATAAAGATATTCAGAAGATTA
ATTTTTGTTATAAAGTGTTTAATTAAAGTTCAAATCAACTTTTGTATTTTTAATTTTATA
ATTCTAAATGGAAAAACATTACACTCCTAATACCTAATTAAAATTGATTTTACATTTTTT
ATGAAACCTAATTTTAAAAAGAGGAAAAGAAAAATACAACTGTAAATAATTATTCTTATT
CAAACATAACATATACCCAAAAAAAAAAGTTAATTACCATAAATAAAACTCTAAAAATAC
TTATTTTAAAGTTAACTTTCACATTCTCAACAATCTCACCTAAAATCATAATTCCATTCA
TTTATAAAATCCAAACCAAACCAAGTCAAAACAAAAACAAATCACGTCAAAAACATCAAA
VfGBP1 promoter
>prV. faba_jg123098.t1
SEQ ID NO: 100
TATATATGATACATTATCAAAAAATTAATTTTAAATAGAAATAACCAAAAAAACTAAACA
AAAAGTTTTTTAAATAACTCTAAATGCTTGTATTTCCTAAACCGATAAAAATATTCAAAA
GAGTAATTACCATGAAAAAGAAATTAATTAAAAACTTTAAAAACATATTCTTTAAAAACT
TTAATTACCATAAACAAAACTCTAAAAAGTTTATTAGAAAGTCAACTTTAATATTCTCAA
CAATCTCACCTAAATTCATAATTTCATTACCTATAAAAACCAATCCTAACCAAAAAATAC
ATAAACCACATA
TpGBP1 promoter
>prT. pratense_Tp57577_TGAC_v2_mRNA26446
SEQ ID NO: 101
TTGTGTGTTTGCCATGGAAGTTTTCTTACCCCTGCTGAATTTGTCAAGCATGCTGGTGGA
GGTGATGTGGCCAATCCATTGAAGCATATTGTTGTTAGTTCAAGCTTAAATTAATTTGAG
GCTAAAAGAGAAGGAAACTATGATATGAGTAATCATATTTTTATTTATTATTATTGGTTC
ATTTGTTCAGTCTTAGACTTTGAAACCATGGATGTTTGAGGATCTGAATTTGGTGTTTCT
TATTGAGGTATATTTTTACCTTGGTCTTATGTATGTTGTGACTTTGGGACCTCTATGAGG
TGAAATGGTACTTGCTAGAGTTTAAATTTTATGTTAATTAATGTTAATTTAATTGTATTT
TTAGACTATTTAGTATCTCTCTGTTTTTGTGGTTAATTTTAGTTTTTTTTAAATGTTTTA
ATATATAATTAAATTTTATTATCACTTCTATTTTGAATTCATTTTTATTGAAAAAATTAT
TCTTTTATTATTTAACAATTTATCATGTTAAAAGCTGATTTTACTGAAAATAAATTATTA
CAATTTTCCTCTTTATTTCTTTTGCATTTTCGTTTTCTTTTTTTGATAAATATTATCTTG
TATTTTCTAAAATTTAAAATTGGCTAAATAGGGTTTTACCCCCCGCAAAATAGGTCAGTT
TTGTTTTTCCACATGAATTTTTTTTTTGGATTCTCCCCTGCAATATGAAGATTCCATCGT
TTACCCCCGGATGGCCAAATTGGATTGACCGTGTCAATCTGCTTATGTAGCATGCTGAGT
CAATTTTTTTAATTCTTTTTCATTTTTGTTTTTTATTTCCAACATGGATTTCCTTTTATG
CTCCCATGTTATGTCATAAAAAAAAAAGGATTTCATGTCCAGTTGGCCATCCAGGAGGGT
AAACAATGAAATCTTCATATTGCAAGGGAGAAATCCTAAAAAAATAAATTTGTAGGAGGT
AAAACAAAACTGACCTATTTTGCAGGTCTATTTACCCTTTAAAATTTTATTTTTTGTGAT
ATTCTTTAGACTTTATTCAACTATGTCCTCTTAGATTTTTCATTCTAAATTCGTTTTTAT
TAAATAAATCAAGTATTATCTCATAAAAACTATTCACAATTATTAAATTCTCATTTCCTA
TTATCACTTGAGAGTTATATATTTTGTTTATATTCTCCTTAATTTGACATCAAAGTTTCT
TTGAATAGAAAATTCGAGATTTCATTTGAATAGATATATGGCTGAGTTGTGTATCATCAC
ACTCATATTTAGAGTAAACCACTCTACTTATTGCTCCTTAAACTCCTACCCGAACCTACT
TTTATGTTTAGACTCGACAAGTAGAGAAATTGAAAACACATATGGGCTTCAGGAATAGGC
CTCCCACTCAGGAAAAAAAAATCACAAATTTAAATATTACCAATTTGGTTTTTATTTGAT
TCTTATTGAATTTTTTATAGAGAATATCCAATGGGTGACTTAATATAATGTTATTCTTAA
GTAGATTCACAAATTGTGGCCAAAGGTGTTATGTTGTCCTTTCTTCATATCTTAGTTTTT
GGTTTTCATTCTTAAGTAGATTCACATTTTTTACTTACCCTTCATCTTATAAAAAGAAAT
ATTTTAGAATTGATGGACCTTTATTGTTCCTTTGCAATTTAGTAGTTCAGCTCCCACCAA
TTTTATTTTAAATTTACACTACTGTAGCATGTTTATGTTTATTAACATCCACTAAATTGG
AATAGCTAGCAGCGTGACTTTTTTATAAATTAAAAAATTAAAAATAGAGAAATTATTGAC
ATAGACTTAAAAAGAAAACATTAACAGAATTGAGTTTTGTGCTAAGATTTGAAGTTTTCT
CCACAATAGACTTAAAAAGAAAATAGAGAAATTCTGTTTTAAAATGCAGGTACCATTTCC
CCCACAATGACCGTAATCGAGTTAGATTTCTACAAAATCAAACCCCACTAAATTTGGAGT
TACAGAGAGAGATTAATTACCGTTTTAGTGAAGGTATGTCTTCCAAAATTCTGCACAAAA
TCTGCCTTCGAGTAACAACTTCAAAGCTTCGCACACAACTAAATTTGGATCAATAATTCA
CCAAACGGATACCGAAATGACCGTAATCGAGTTAGCTTTACACAGAATCAAACCACACTA
AATTTGGAGTTACAAAGAGAGATTAATTACCGTTTTAGTGAAGGTCTATCCAATTACTAA
TTTTGCAGAAATCTACTTCTGACCTTCAAATTCAACATCGTATACCAAACACATCGTAAC
TCCAAATTCGACGAAATTGAAATGATAACAAGGGTAATTTTGTTAGCTTTCCAGACATGT
AAATACTATTAAAAAATGAGCTACAGGGTGAGACATGACAAAAATATCAGTAGTAATTCC
ATACAATAATTAATTTGGACAGACCTTCACTAAAATGATAATTAATCTCTCTGTGTAACT
CCAAATTTAGTGGAGTTTGATTCTGTGGAAAATTAACTCGATTGCGGTCATTTCGGTACC
ATTTTGGTGAATTATAGATCCAAATTGAAATCTGTGTGAAACTTTGAAGTTGAGACTCGA
AAGTAGAGGGTAAAAATCCACATTTTAAAATATGGGGGTAAAATTCTGATTTAATCAAAA
TAGGGGGGTCAAAATTGTATTTAAGCCAATTATCTATTTTTACATAAGGCATCTAGAAAT
TTTGACGTGCTTTCATAAATCCTCATTGTAGTGACTCGAGTCACTGCTAAATATTTATTT
AACTTTATTATTATTATTATCATAGTATTTTAGTCAAAAAAAATATTAATATACTAAAAC
AAACTCATTAAACAATTGTTCAAAAAATTAAAACCGATTGACAATGCAGTTATTCTTATA
TTTCTCAAAAGAGTAATGATTATTCATTTTATTTTTAAGGAAAAGAGTAATGATTATTAG
GAAGACTCTAATTGAAATTGTAATATTATCTTATACTTATTCAAACATAAGTTACACAAC
GGATATATACTTTACATTAATATCACCATAAAAACTTAAAAAATTATTCCAAAAAATAAA
GTGTTTTATAAAATATTTTGTTTAAAGTCAACTTTTTAATTCTCAACTATCTCACCTAAT
CTCATACTAATCCATCTATAAAAACATAATAGGAAAAACAAACACCACACCACACAACAC
AAACATTGTAACAATCTCTTATCAACCAAAACCACA
TrGBP1 promoter1
>prT. repens_CM019102.1
SEQ ID NO: 102
ACATCTAAAAACATCATTCCATTATAACGTCCTCTATTTAAGTAGCGGACGACATATTCC
AAAAACTTCTCATTTTTATAACATACGCTACTTAAGTAGCGGAAGGGTAAAATAAAAAAC
GCGTAGGGTTCGTGAGTAATCGATATGGTGTCAAAAGTAAATTTCATTGCTTAGTGTAAT
GAGCTTATGCATTATCCAACAACAAAGCCTCTTTAAAAATGAGTTAAATAGCCCAATAGC
AAGGTCCAAGTCCAAGGTCCAAGTCAAAAGACATTAAGTCATTAACTATATTTTCCCCCA
TCTTGCAGACCAGGGACAGGAAAATGGACCAATTAGCGCGAGTGCGCGCACACACACACA
AAGAGAATCAAGAAAGGTGGAAAAAGCTTTAACCTAATACCTATCTAGGACCTAGAGGAA
TTTCTTAAAAAATAAAACTTTTACAAGAAGAGACTAGTCGAAAAGGATAACACCATTAAG
AGACTACAAATGCCCCATAACATGATACTAAGGCCAAGCCCTTTTTAAAACAAGGAATAA
CTCTAAAGGCGGTGAACAATGAGAATAACCCAACAATTAAAATGCACAGTGAGTTGCGAC
GTACCTGAAGCACCTAAAAGTTTTATACTAAACTTGACTTGTAAATTGACAAGTAACAAA
TTAGGGCTTTGTTATTTGGTTATAAATTCTCATTTTGCCACCCTACTCATTCTCTCGTGC
GCCCTATGATGTTATCAAAATACATCTTTCCGCTACTTTGGTAGCAGATACACTCAAAAC
ACCCTACCATTATAACATTCTCTACTTAAATAGCGGACGATATTTTCCAAAAACTTCATT
TTTATAACGTCCACTACTTAAATAGCAGAACGGTAAAATAGGAAACGCGTAAGGCGCGCG
AGTAACCGATAGGGTGACAAAAGTAAATTTCATTGATTAGTGTAATGAGCCTATGCATTA
TCCAACAACAAAGCCTATTTGAAAATGAGACAATTTCTCTTCCCACCCTCCACATTTCTT
TTTCATCCCTATAGCATGCAAAAAGAAACTTGGGGAGTGGGAAGAGAAAATTTCTGAAAA
TGAGTTAAATAGCCCAATAGCAAGATCCAAGTCAAAAGACATTAACTATTATTCTAAAAG
GTGATTGCTCATCCCACCCCCCTAACTCTTTTTCACCCCCCTAAAACCCAAAATTACCCC
TGAAAATTCAAAAAAATTGAACGAAAAAGTACGGATTTTGCGATCCGTGCTTTTGAAAAG
TATGTATTACAAAAGTAGTGCGGAAAAAATACGGATGACGTAGTCCGTACTTTTCCTTAA
TAAACCCAGAACATCAGTCTTTCTTCTTCACTTTCTCTTCTTCACAAACACACAATCAAA
ATCTCCATACGTGATCCTTGCGCGATTCCGTCCATTGGAGGCTTAAAATCAACATTGGAG
TGTTGCCTAACTGCATCTACACGCATTGGAGCTCAAAATCAAGGTAAATTTCGTAACCCA
CCGTTTAGTTTGCGCTTACCTACTTTCTAGCTTACTAGGTTTCTGACACACTTTAGCACG
GATTAGGAGATCCGTACTTTAGCACGGATTATGCGTGCCGTACTTTTCTGGGTTTGGAAT
TTGCTGCGTAGCTCACTGACTTGCTTATATAATCTGCAATATTGTTACATTAACTTAGAA
AACAAGTAACCTAAGGACTATTTGGTGCATTGTAGTAGTATTTAAACGCCAATTTCATGC
TTAGATTATTTTTATGCCAATGTATAAACCATAATTAGTAACTAATGTAATTCAAAAATA
TGCATAAACTTATGTTATATCTCTAACCAAATCAAAATTCAAAATTTCACCAAAACCTAA
TTAAATATAACATTCAACATAATAGACATTCATCCTAACAACCAATAATCAACAATGTAA
ACAAAATCATTTACAGCATACACATTCAACCCTAACAACCATCCATTCATCTCTAAACAA
TTGAAAAATCAAAAAATAAACCTATACCTAAACCTAATCAAAATAATAAATCAAACTAAC
ATGCATTCATCATAGTAAACAAGAATCAACATCTTCAACAACATAATTTCAAATTAATCA
TAAATCAGAGATTTTTCATACATGCAATCCCAAAAACGCAGAAAATTGAGATGAAGAAAG
AGGAACACAATAAAGTGATCAAAATTAGAAATTAACTTACTTGATGGAGTAGGAATTGAT
AATGTATATGGATTCCGGCCTCCAAAACGAATGGTTGATGCTAATTGATAAACTTGGATG
CTTAGGGTTCTTATTTGTTCAAAACCCAGAAACGTTGAAATGAGAAGAGGAGTTAACGCT
CTGTTTATATTAAGGAAAAGTACGGATCTCGCGTCCCGTACTTTTTCCGCAACGGATCTT
GTAGTCCGTACTTTTAAAATAGTACGTATCGCGTTTCCCGTACCAATTTTCAACGTAGGG
GGTGGAAAATTCATGTAAAAATACGGCTCGCAGGGAAACTCGGGGGTGGAAAAGAAAACC
GGGGGGGGGAAGAGCAAACTTCATTCTAAAACTCATTAAGCAGTTGTTCAAAAAAATAAA
ACCGATTGAGCATACAGTTAAATGCTTATCCTATTTTTCAAATTCTTAAAAACATAAACA
TTCAAGAGTAATGATTATTATTTTTTTTCAAGGAAAAGAGTAATGATTATTAGGAAGACT
CTAATTGAAATTCTAATATTTTACTGTACCTCTTCAAACATAAGTTACATAACGGGCATA
TACTTAGAACCAAAAAAAATAAATAACGGATATATACTTTACATTAATATCACATTAAGT
TTTATCAAAAAAATTAGTCAAGTAAAAGTTTTCAAAATAAAAAAATTGATTAAAGTCAAC
TTTTATATTCTCAACTCTCTCACCTAATCTCATACTAATCCATCTATAAAATCATAGGAA
AAACAAACACCACACAACATAAACATTGTAACAATCTCTTATCAACCAAAACCACA
TrGBP1 promoter2
>prT. repens_CM019114.1
SEQ ID NO: 103
AAGGCCCAACTTCTCAAAGTTGCTGCTGCTGAAACCATTTATATGCTTTGGAAGTATAGG
AATGATATTTGTTTTGACAACCAAGTACATAACACAAAGATAGAGGAAAATATTATCAAT
ACAATAGTTTATAGAGGGTGATGTTATCCTAAGCTTAGAAAACATGTTGCTCTTATGCTA
ATCTAGCTTAGTTTCTCTTTTTGGTTTGTTTGTCCTCTTTGATGGTTGGATCTTTAGATC
ACCTTGTACTTACTTACTTTTTTGAATGGAATAAATCTCTTAATTCAAAAAAAAAATAAA
AAAATAGATGGCTGATTTGTGTATCATCATCAACTCATATTATAGTAAACCATTCCACTT
ATTGCTCCTTGAACCCCTATCCAAATCTAATCTTTGTATCACAAAATGGCCCATAACTCA
ACTTGTACTACTAGAACAAACACAATTGTTAACATGCATGTTTGCATAGCTCACAATGAG
ATTCTCCACTTCATATCTAATCAGATTTGTTGCAGTGTGTCAATATGATTTATAAAGGAA
AGAGTGATAAATAATTTGATTGTAGTAATTATAAAATTTAGTTCTAAATTAAGTCAAAAT
ATTATCAAGAGATACAAATACACATTATGAAAAAATTTGATTGTAATGGAAGAGTCTAAT
GTGCGACTTAATAGTATAATGCTATCCTTAAGTAGATTCACAAAAATATTTTGTGCTCCC
TTCTTCATATCTTAGTTTTTGGTTTTCATTCATAAGTAGATTCACAATTTTTTACTTGTC
TAGAATCATTGTGCGATTTTTACCCTTCATCTTTTAGCAATGTGCTTATTAAAAAAGAAA
TATTATAGAATTGATGGACCTTTATTTTTCCTTTGCAATATAGTAGTTCAGCTCATACCA
ATTTTCTTTTAAATTTACACTAATGTAGCATTATTTATTAACATGCACTAAACTGGAATA
GCTAGCAGAGTAACTTTTTTTTATAAATTTAAAAGTTTAAAAAAATAAAAGAAATTATAA
TTGACATAGACTTAAAAAGAAAACATATGCTACAACCTTGTTATTATATTTTTATTTTAA
AGAATAATCACGCGACTAAAATAAATTTGGTTGCACGTTAATAACTATGCTACCAATTTG
AAAGAAAAAAAAAATAGAAGACTATCAATGTTTGGCTCCTTTGTCTTTTTCTTGGAAAAT
GACTCACAAGGGATATCTTTAGGGTTTTGATTTTCAAGTGCATCTTTTTTTTTTTTGTTA
ATTTCAAAACGAACACTTAATGAATTAGACTTTAGCATATCAAATGATTGAGAGCTTTAA
TTCCTTGTCTTTCTACCATATTTTTCCTGTTGTTTTCATGCCTTGTCCTCGTTATTATTT
CCAAAATTCCACCCTAAAGTATATTCACGTTAACTATGCATGGACGTACTAGTAGTACCC
CACACGTTTATAACTTATGTCGCTTTCATAATATATTCTCTTCAGCGACGTAAGATTCTT
GCATCCTATATATTCTTTTTTAGGCGCATCCTTTTCTGACTTCAACAAGCGCTTATTGAG
TTAGAATTTATCGGATCCAATGATTGAGAGCTTCAATTCCTTGCTTTTCTATCACGTTTT
TCCTTTTGTTTCCATGTTTGTTCTCGTTATTATTTCCAAAATTCTTTCTTAGCGCATCTT
CACGTTGACCATACATGGATGTACTATACCTCACGCGTTTATATCCTTTGTCGCATTCAC
AATACATTCTCTTGAGCAATTAATGGTTTAAAATTCAGCTAAGAAAAATTCTTGCCTCTT
GTATATTCTTTTCCTAAATGCCCCACTTCTAAATAAGGTATATTCTCCCCCAAAATAGAA
AAATTGGATTCATTGCCATTATGACCAAAACTAATGTTACTTTGAACTACCTATCTTCAT
TCCTCATCGATGAATAAATTTTGAACCATTATCCACAACTGTGACGACATTCCATTCTAG
AGTTTCACCATACTTTCCCCCCTCTTGCAGATCACGCACACACGCACAAAAGAGTCAAGA
AAACTTAAAAAAGCTCTAACCTAATACCTATGAATGACCCAGAGGACTTTCATAAAAAAA
AGCTTTTACAAGAGGAGATGAGTCGAAAAGGATAACAACATTTTTATAACGTCCGCTATT
TAAGTAGTAGAAGGGTAAAATGAGAAACGCATAGGGCGCGCGAATAATCGATAGGGTGGC
AAAAGTAAATTTCATTGGTTAGTGTAATTAGCCTGTGCATTATCGAACACTAAAGCCTCT
TTCAAAATGAGTTAAATAGCCCAATAGCAAGGTCCAAGTCCAAGGTCCTAGACAAAAGAC
ATTAACTATTATACTAAAACTCATTAAGCAGTTGTTCAAAAAAATAAAACCGATTGAGCA
TACAGTTAAATGCTTATCCTATTTTTCTTTTTTTTGGTACAAAATCCTATTTTTCAAATT
ATTAAAAACATTCAAGAGTAATGATTATTATTATTATTATTTTTTTAGGAAAAGAGTAAT
GATTATTAGGAAGACTCTAATTGAAATTCTATTATTATTATTATTATTATTATTATACGG
GTCATGCTAACTAGTGCCCGAGGCACTAGTTAAGGATACTAAAAAGAGCAAGTTTTGCAT
TGATAATAGTATTCTTTATACTTTAAAAAAGTAAAATACACAAGTTCCAAAACATTTTTT
ACTATTTGTAGTTCCTTAACTAGTGCCCCGGAGTAATAGTTAGCATTTTCCTTATTATAC
TGTACTTCTTCAAACATAAGTTACATAACGGATATATACTTTACATTAATATCACAATAA
GTTTTTTCAAAAAAATTAGTCAAAAAAACTTTTCCAAGAAAATATATTGATTAAAGTCAA
CTTTTATATTCTCAACTATCTCACCTAATCTCATACTAATCCATCTATAAAAACATAGGA
AAAACAAACACCACACAACATAAACATTGTAACAATCTCTTATCAACCAAAACCACA
TsGBP1 promoter
>prT. subterraneum_Tsud_chr4.g17370.1.am.mk
SEQ ID NO: 104
ATACTTTTCCCTATTGTAGATCAAAGATGGCAAAATGGACCAACTTATCGCATCGTTAAC
TCATATTTCTTTTAAACTTACATTAATATGGCATGATTTAGTAATCTGCACTAATTTTTT
GACATATCTATCAATATGGCTTTATTTTCATTAAAAGAAAAAAAAACACACAAAATAAAG
AAATTACTGACATGGACTTAAAAAACTATGATACAAGCTTATTTTTAGGTTTTATTTTTT
AATTTTAAGGAATAGTCATGCTAAATAAAACAATTAAAAGTTTGGTTGTACGTTAATAAT
GATTCTACCTAAGCGTTAATTTGAAAGAAAACATTTAGTGGGAGACTGTCAATAGTTTGC
TCCTCTGTCTTTCCTTGTGAAATGACTCGCAAGGGATACCTTTATGGGCTGATTTTTAGG
CGCATCCTTTTTTGACTTCAACGAACGCTTATTGAGTCAGACTTTATCAGATCCAATGAT
TGAGAGTTTTAATTCCTTGCTTTTCTACCATGTTTTTCCTTTTGTTTCCAAGTTTGTCCT
CGTTATTATTTCCAAAATTCTTTTTTAGCACATCTTCAAATTGACTATACATGGATGTAC
CGGTACCACATGCATTTATATCCTTTGTCGCATTCATAATATATTCTCTTGAGCAACTAT
TGGTTCAAAATTTAGCTAAGTCAGATTCTTGTCTCCTATATATTCTTTTTCTAAACGCTC
CACTTCCAAATAAAGGTATGTTCTTCCCCAAAATAGAAAAATTGGATTCATTTCCAATAT
TTCCACCACTAATGTTACTTTGACCTACCTGACCTTCATTCCTCATTGACGAATAATTTT
GGAACCATTGTCCACAACTCTGACGACATTCCATTCTTGTGGGAGAATGGACCAATTCGC
GCGTGCACGTAGACATACAAACACACACACACACACACAAAAGAGAGTCAAGAAAGACAA
AAAAAGCTCTAACATAACACTTATCCAGGACATGGGGGAATTTCTTAAAAATCCATTTAC
AAGAAGAGAATAGTTGGAAAGGATAACACCGTTATGAGACTTCAAATACCCCATAACACG
ACATGTCAAACTATGGCCAAACCATGTTTAAAAACGAGGAATAACTCTAAAGGATGTGAA
CAATGAGCATGACCCAAAAAAGTGCAATGCCTAGTGAGTTGTGACGTACCTGAAGCACCT
AAAATTTTATACTAAACTTGAATAGTAAATGGACAAGTAACAAACAAATGGCGCACTGAT
TCCTGCGCAGGAGCACAAAAAACACATGTTGAACCCAAATTGTCCTGAATAACTGATAAG
TGCTAAAAAAACAGAAAATTTATGTATTTATTTCAAGAACCTATTAACTTGTTTTACACA
ATATTGGTTTAAATTAGTGAATCAGAAATTTTATAGCATTTATACCTATATCTAACTATT
TTTGTAGGAACATGGAAGCTTAATGGAATTGGACTCAAAGAACTTAATATTTTGATCAAG
TAACAGTCGTTGAGCTAGCAGTAGCAAGCTGAGTCAGAGAGTCCAGGGAAATCCAACATA
AGGTTGGTCGCTAGGCGAGCAAGCAAGGAGCGACGAAAGTCAGAGAGTTACATTTTTGGA
TACTAGGGTTAGTCGCTAAGTGACTAGTAGCGACTAGCGACCATGGAGCGATGAAAGTCA
GAAAGTTCCAATTTTGGATATTAAGGTTCGTTGCTAGGCCAACGACCATTGGCAGTTCAG
AGTAAATATTTGTTGATGCGGCTTTTCCTTCCATTGAAGAAAGCTTGGTCGCCTAGAAAA
ATATGTATTTTGAGTTTTTGTGCACAAAAGGTTAAAATAAAAATATGAGAAACTTTTCGC
TAAATACGTGTACGCGAGACTATTAGTCGCAGATGCAATTTAGCGTGTTTTCAAAATGGA
AATTTCGCTAATCGCGCGTATGTGATGCTACACTTAGTGCACGCAAAATCCACATAAATC
ATGTATTTGATAGAAATTTAATGAAAAATTAAGGGTTTTTAGACTCTATCTTTGATGGCT
ACAGGATTGTTGAAGAGTTAGGGCTTTGTGAGCTTTCAAACACCAACCATCTTAGATTCA
TTCTATCTATTACTCTCATTGGTCCTTAATATAAGAGAAATTTTATATTTTAGATTCATT
GAGAATCTAATGTATTTAGTCTTTATTATTCACTAGATACATTAGATTCTTAATGAATCT
AAAAAATAAAACTTCTCTTATATTAAGAACCGGATGGAGTAATTCATTACTTCAAATCTT
TTAAATTAGTATTCATTTTTAACCTTTGTTAATTGGTTAGTGTAATGGGCATAAATGACT
TATTCCAGTTATCCCATGCATTATCCAACAACGAAGCCTACTTGAAAATGAGTTAAATAG
CTCGAAAAATTCCCTTCCCCCCAGTCCATATATTTTTTCTCGCTCTGAATTTTCATTTTT
GCCCTTGAATAAAACTTCGGAACGCGTTTTTTGAATTTTTTTCCTTAAATACAAACTTCA
GAACGTATTTTCCGAACTTTTTCCAAAACTTTGAAGTAGGAAAACTTCGGAAAACACGTT
ATGAAGTTTTTATTCAAGAGCAAAATTGGAATTTTGAGGAGGAGGAAAGACATGTAAGAG
GGGAGAAGAGAATTTTTCTCAAATAGCTCAATAGCAAAGTCCAAGGTCGACATAATTATA
TCCTCAACATCATGTTGATCAGATATCGACCCGAGTCACCACTAAAAAATTATTTAAATT
TAAATTTTATTATTTTTATTATTATTTTAGTCAAAACTAAACACATTAACTGTTATACTA
AAACTCATTAAGCAGTTGTTCAAACAAATAAAACCGATTTTTTTTTTGGTAAAAACAAGG
CTAAAGAAAACATTCAAATAAAACCGATTGAGCATGCAGTTAAATGCTTCATATTTTTCA
AATTAAAAACATTCAAAAGTGAAATGATTACTATTTTTTTTTAAGGAAAAGAGTAATGAT
TATTAGGAAGACTCTAATTGAAATTCTAATATCACACTTCTACAAACATAAGTTACATAA
CGGATATATCCTTTACATTAACATCACAATAAGTTTTTTCAAAACAATTAGTCCAAAAAA
TATTTTGATTAAAGTCAACTTTTATAATCTCAACTATCTCACCTAATATAATACTAATCC
CATCTATAAAAACATAGGAAAAATAAACACCACACAACACAAACATTGTAACAATCTCTT
ATCAACCAAAACCACA
SEQ ID NO: 105
>prL. japonicus_Lj1g3v3023590.1
LjGBP1 promoter
TTTTTGAACATACATAGCGGTAATTTTGAAGCCATAAAATTTATGTTTTGAAACATACAT
GTATAACAACGATTTTCAGTCGAAAACTTTGTTAAATTTGTTTTTTTTTTACACTTCGTA
TTTAGTGATACCTGAAATTGTCAATAAACAATATATTAATTGAATGTCGTGGATTTCCAT
AGTATACATATAAGCACCGTAAATTAATAAATTCCAACTTACCCATGGACTCAATGTCTA
GTCATATATATCTTTAGTTCAATATTTGTTCAGTTTTGCTGACCCCTTTATACCTTTTTT
TAGGGTTGTCCATATCGGACAGTTTCGGGCCAAGATCGTCAGTTTTGCCGGTTGAAAAAC
ATCAAACCATCAGACCCGCCATCACTTGCTCACTAACCACATGTTGTTTAGGTTCATATT
TTTTGGGTTGTGATTTTGATGGTGAGTTCACGCAGGTCGTCATATCAAGACACTAATCAA
GAAAAGAAAAGAAAGTAGATGAAAGAAACAAACCCATACAAGTTGGATCAACATTCAACA
GGTTCATCAACATTCAACATATGAAACAACACAACACAGAAAAGGTGAAGAGAAATCAAC
ACTCAACAAATAAGAACCTAAACAATAAATACACACAAATCAACATTTTTCATCTTCACC
ATCATCTTCTTACAACGTTCCATTGTAAGTGCTTGAGTGGGGATAGAAAAGAGAACGAGG
TGAAGGAACGTAGAACCTAGAACCGCCTCTACAAGGACGAGATATCATCCGCCGTGATGA
AATCACTCTTCCAATCTGCAACAGTGGGACGAGGAGATGAAGCTTCCACAAATTTGCGAC
TGGGAGAGAGAAAGGGTGAGGATGAGGTTGATGGTGAAGATGGCGGGCGACAATGATAGT
GAAGAAAGTGAAGGAATTAAAGAGAAGGAAAAATAATGGATCTTTGTGAGGAGGTTCTTG
TGGCAATGGGAGAAGAGACATGGTGGTGGTGAGTAGGGTTTTCCGATTTTCTCAAGCGGG
AAGGTGAGGGGCTGAGAGTGATAGAGAAATTGAGAGAGTGTGGTGGCTAGGGTTGGAATT
AGAGGAGTCTCGCATTTTTTTATTATAAATAATACCTAATTTGGTCGGGCTGGAGCCTAG
CCTGAAATTGATCACTGTTAATTACAAGAAATCAGCTATTTGAACCATGCGACCTGTGAA
CTAAACTGACCCGCTCGGTTTCACAATTTTGTTGTTGTTGTTGTGTTTTTACAGGTTTCG
ACCATACCAGATGTTTATGGACAACCCAATCTTTTAAAAAAAATAAGAAGCATTATAAGA
AGCATTGTTGATGCTTCTGTACACATTATTTCAATCATAGAGTCATCATTGCATGTTTTA
GTCAATCGCATGAGCAATTAGCAAATGACCTCACTAGATGTAAAACTTTTTACATGATTA
ATGCTTAATTGCCCATTTTTTTCATTCTTTAGTAGCCCCTTCAACCATCATAAGAAAAAG
ACCCTGTAGACAGCAAAATTACATTTGGAACACCAACAATGATATATACACACCTCATTT
TTTAAACACTTAATTTCCACCTATTTTTGTTTCTACCTATCTCCTCTTATCATCTATCAC
ATCTCATACTTTTTCTTTTCTTCCTATCTCTCACCTCATTCCACCTCTTCCACTTCTTTT
TGAGGTGTGTAAGTAACATTTTCCTTTGGAACACACAATTGGATGAATCAATTTTCTCCA
TATATTATATAATATCACTCATGTGATGTATCACTTAAAAGGAATCCTACATTCTTGCAA
GAATCACTGGGTAAACATGCATAAACATGAAAACAGAATAAAATCGAAAGCTAGCATATA
ATAACATGTCAACTTATGTACATGTACAACGGTAATATTCCCAACAAATTATGACATGTG
TTAATTTTAATAATTTTCTCGAATTAGAGGAGGATTAGTTGTGCAACCCGTTAGAGGAAA
ATAAACACAATGATTCTAAACATATTTGCCATTAGAAAAAGGTATTGAAATGTGATGCAA
TCAAAAGAGTATTTTGTCCCTACTTATAACTTTTCAGCGCCTTGGAAATGGAAACATGTT
CATAAATATCTACTCCAAATCTAATGAAAGAAATAGTACTACATACTCGCTGTCCTCACG
CAATATAAATTGGTTGTGATGGATCAACTGTATGGCTATCAAAAAATCTACATGTATTAA
GAATCATAACATTATAAAAAATAGCAGCATGAACAGAACGGCATGCAACTAATTAAAATT
CGAACATTATATGTTTTTCTCTTTTGTGTCCGGTTGTGATTCGTTTATAAAACTTGTCTC
AAGAGCCAATTCCATTAAGATATGAGAAAAAGTTTGATGCACCGACGGTGTAAAAAATTT
TTTTTACACCGTCAACCAATCAGATTTCAAGGATGTGAGAAAATCTCTCTTTTCATTTAA
TTTCTTTAATTGACATGTCACATCCTTGAAATCTGATTGGTTGACGGTGTAAAAAAACTT
TACACCGTCGGTGCATCAAAATTAAACTCTTAAGATATAGTTATTTTCAACTCGAACATA
TTATTTATATCTGTAATCTTTAATATTTGGAAATACATGTTTGTCAGACATAAAAAAAAG
TACATTTAGTGGAGGTGTTGAAATCGCCAAATGAAACACGAAATTATGTGACGGACTTTC
TCACGTATTCTAGAAAAATGTTTAAAAATATAGAAGAGTTTAGTTCAAAATAAACATGCA
ATTTTGTGAGAGTGCATAAACCGTAAAAAAAATGTTCTTTTTTTCTTTAGATACTTTGAC
GAAATGTAGCCATAAAAAACTGTCACAATCACCAACAAGATCCAACACAATTTATAAATT
TCTGAACTCTTTAATCATTTAGTTCTCATAGTTTAATCACCTAATATAATTTTTGAGTCT
CGAGTAAATTAATCTTGTATACAAATCATTATTGTTTGACAAATAGAATGGCTGTGTACT
AATATTTGAACTATATA
LaGBP1 promoter1
>prL. angustifolius_OIW16739
SEQ ID NO: 106
CCACAAAAGTCTATCTATACACATGAATAGTAAGGGGAAGGAATAAAACTCATAATATTA
CAACTTGAATATTCTATTGCCCACCTATTTGCATAGTAGAAAATATGTTTTAGGTTAAAA
ATCTCACCTTTAAAATTCACATTATTTTTCATTTTCCAAAATGATAAAAAAAGTGAATCT
CATTTTATTAGTACTTATGAGAAAATACAAATGATGAGTAACTAATACATTAAATATAAT
TTCACACATTAATTATAAGTGATATTTGATTTATCAAAACTTTATGTTTTACTTATAGTC
CAAAATGTGTATCATTATATACATCATTTTATTCTCATTGATTAAAATATATAAAGATAA
TATATATTTAATTATATCTTGTTTTTCTAACTAATAAAAATGAGTTTGATGTGAAGTGAT
ATAGCAAATGAGTCTCTTTTATTTGTGAGCATGTCTTTTATCCACTATATGTATTATGTA
TCTTTCATCTTTGTTAGTTTTTATATAACTAACGTATCATATCTTTTTATAGATAAGGAA
CAAACCATTTTTAAGATGGGAATGGAGTATCTCCTTACATAAGGAGATAATTTATCTCCT
GCCAGTGAAATCATGACATGTGTCATGTTTCACCATTTTCTATAAAAATATTTTTAAAAA
TAAACAAAATAGTACACATGTCACATTTTTATTGGTTAGAGGAGATAAGTTATCTCCTTA
TATAAGGAGATACATTATTCTCTTTTAAGATATAGTCATTCCTCTTAACAAAACGTTTTG
TAAACAACTTTTTTTGTTTGGTTAGAAAATATATAAAGTTACTATATATTTTATTTATAT
TTTATTTTTTCTCAAATAAAAATGAGTATTATCACACTCCTACTCATTTTACTCGAATAT
ATTACCTTAATTGTCACCATGTGTTAGGCCACATGCATTGGAAAATTCGTTGTTTTAAAA
TTTGTAAACGGAAATGACCAAAAAGGACGTGTCACTCAAAGCATTGTGATGCATTATGCA
AAGTTGCACGTACTTGTTTGTTTCTTTTCGAACGGTCATGTCCAACGTTTCCAACGCGCG
CAAGTAGCGAATATCAATGTAATTATTGTTTTTTGGTGCACGGAAAAAGGAATAAAGAAC
ACTACAATATCAACGGCAACATTTCTATATAATATATTATAATAATATGTAACATACAAA
ATTCAATTAATTTCATATTAATTATTTATCCAACGGTCATCAAGATAATACATTTTGTAA
CACAAAATGAGTTATTCTAATATAATTTTTGTAAAGGACCATGTCAAGTTGTTTGTACCA
ATAATTCTAAAATCTCAACCTCTTAAAATTGATCCTTCTATTCAAGATCTTTTATAATAT
AATTTTTATCTCTCGATTACTATCTATCTTTTATTTACACAAGCGTCTTCACCAAGATCT
ATAAAAATATTATTTTTGGTCCAAATTTCATGTATTTCATAATGTATCTCTTGGTCAAAG
AAAAACATTTATAGCTTGGAAGACTTTTTTTGTGCGTTTCGTGTTGCTTTATTATCCATA
TATTTCCTTACTTATGGTCAACCAACTCATGCCAACCCAAAAAATATTTAGGATATATTC
TCAACTATACTTTTTTGGTACATTATTTATTTCCTTACATTGATGCCTATTTTAGACCAT
CTTTTTTATTGTATTCTATTTGAGTTTATTATTATTATAATTTATTTTATTCATCCGACG
AGGATATAAACTAAAAAAATAGAGAATATTTGTTAATATTCCACAATTACTATTGTGCAA
TTTATATCTCATAACCCAATTTTTTATTAAAATAATAATTATTGAATTTAATTATTTGCA
TCATTTCAACCCAACCTTATTTGAAATGTCACAATGCTGTACACTTTTTTTTCTCTCACC
CTCCGATTTATAATTCTTCTAGTTTATTATTTTAACTTTAGGATGAGATAATATTTTGAA
CATGTTTTTCTAATCTTCGGATGGAGTAAATTGTATCTCTAAACTCCAAAACTATTTGAT
CCCTACTGGTAGAAATATGGAATAATGAGTTTTGCCTTTTACAAACAATTAATTTTTTCA
TTTAGAAAATAGATTTCAATTCACGTACAGTAACAAATATCATTTCAATGAGTTATTGAC
TCTCCTTTTACTACAAAAATATTATTTATTATAAATAAAAAGATAAAAGTGAATATACTA
TTATTTTTTTTTTTTTACCTTACTTGTCTTTTCTATTAATGGATGACCATACTTATTTAT
GGACAATATTACATATTTTGTTGCATAATTTGTACCATTTCATATGGTATAAGTTATGAT
TGGTAAAAAAAATACTTAATAATTAAATTTTAACTTAAATATATTTTTTAACCAATCACA
ATCTCATGAATAAAATGATATATTTAAATTAGAATTTAATTTTTAAGAGTGTCTTACCTC
TAACAAATCACATAAATAAATAGAATGATAAATAATGGGTGCACATATGTTGTCCCATAT
TTAGTGTTACTTCATATGTCATAACCTTGTGATTGGTTAAAAAAATATATTTAAGTTAGA
ATTGAACTTTTAACTAATTTTCTAACAAATCATAAAATCATGTTACGATACTAAGTGTGA
GACAAACCTATACAAAATGGATGGGGATCTGTTGTCCCCAACCATGTTACCAACCACGGC
CAATCACAATGAAACAAAACTGATAAAGTTATGATTATTTAAGTTTTTTAGTTGACGTGA
TATAATATGATTGATCTGAGACAGAGTTTGTGATAGAGTTTGAGACAACAGATCCTTATC
CATACAAATGATACTTCTTACCTCCTTTATAAGTATGTCCACTTACTCAAATTATACCTT
CAAAAGATTTAAATATCCACCACTTGAATTTTATCTTTGTTGTCATGAATCCTAAGCCCC
ATTAAAAGGAGGACAACCCCATTGAACATTACAATCAACAACTCTCACCCACAACCAACA
LaGBP1 promoter2
>prL. angustifolius_OIW17321
SEQ ID NO: 107
AAGATTCTTCATTAATTAAATTAATTATGAATATTTTATGATGATTATATAAAGTAAAAA
TACTTAAATAAATTTTCTTATTTATATTTGAAATTAATTTTTAAAATATTATATTTTAAA
GTTGTTGTTTCTTAATGCTTCTTATTATAAGAAATCATTTTAAAATATTATGATAATAAT
TTTTAGAGTAAATTATACAAACACTCATTGAGTTTTAGTAAAATTAAACAAATATAAATC
ACCCTTTATTTATACTAACGTGTGGACATGCACTACCGTGCCTGTCAACCCGCTTTCATA
TCGTTTGTAATGTATAGTTTGTATTTTATAAATTCAAATATAATTTTTTGCCCTAAATTT
ACGGTAAAATTTATTCAATTTGTCTAATATTCAATTTTTTTAAATACACTTTTAAAATAA
GAAGATAATAAAGATAAAATAAGAAAATAATATTATATGGTTTTTTGGTAGAAATAAAAA
GATATTAAGGGCAAAATAGGAAAACGCAAATTACACCATGATGGGTGGTTATTTTGTGTT
TTCCCTTTATTAATAGTATAGATATATACATTACATTGACGAGCCCCATAGGTGAGATAA
ATGTAATATTGAAACAAAAAGAGTGATAAATATAATCAAGTTAAATAAAATATAATATTT
TGTGTAAACAAATCCAAATAGTACAAGAACCAATATTTTATTATTATTTTTGATTCTTTA
TAATATAGTATAAGAGTTATTTAAACATCATCTTCAAATTTTGGTGCAGTTTGTAACAAA
CTAAAAATAATAAAGATATTGATTACTACTACAGGAGAAATAAAGAGTGACGAGTGTAAT
AAAGTTAAACAAATTTTGCTATATTGTAGAGATTAATCCAAGTATTATACGAATTATATA
TTTCCATTATTTTTTGTTTGGTACAGTAAAATGTAAGTGTGATTTAAACAATATCTCCTT
TAAATTTTGTATACTATTTGGATTTCTCTCCACAATTTTTTTTGTTTGTCACAATATAAT
CGATTCGTAAACTATTTGGATTTCTATCCACAATATATCATATTTTATTTTTAACTTAAT
TATGCTCAACACTTTTTTGTTTAAAAAATCACACTTATTTCTCATTGAGAGGTAGATAGT
GTAATGTATAAACATAAAGAGGGGCAATTTGTATATGTATAAAAAATATGAGTGATTTAT
GTAAGTACACTAAAACCCAGGGGATGCCAATATACTTTATTGTAATTTACATTTTGATGC
AATATTTAATATTTTCTTTTATTTTTAATTTGTTTATTTATAATGAATAAATTTGTAAAA
GTTTCCTCAATATAAAAAAGGAGAGAAAATATTTATGAACAATGTTATTTCCCACCCTTT
CCTTTAGTATAGTTTATACTTATATTTAGTGTTGTGATAAACGAATCGGACCGGAAGTCT
GATCGGTTTGACCAGTAACTAGACTTGAAAATGATTCTGATTTATAGTCTTAATCGAGTG
AGGGATTTGACCACGTTGGAATGGAGAAATTTTGGTCCAACCATATGAAACCCGTTTGAA
TCACAATAACCCTGGAAGTAAATTAGATAGTTCACTCAAAAAATTTAAAAAATGAACACT
AATCTAATGTTTTGTTTTACTCTCGTCCAAAAAGACGGAAAAAATATTATTTCAATATAA
CTATATTCATTTATTGATTATATAAAATTTTGTACTTATATATAATATATATTTATAGAT
AAAAATGATAAATTATATATAAAATCATCTGATTCGACTAGATGGTTTAACTGCTTAAAT
CGATTAAAAATAATATAAAACAGTTTCGAGTTTGATATTCGATTCAGGTTTCACAATATT
GATTCTATTACAAGTCCAAAATGGAATGTCATAAAGTTAAATTATGCTGGTGTAATTGAT
TGATCGTACAAGATTGCATTCCCTCTATAGTATATCTCTTAGTCAATATTGAAAAAGGGT
TTTATTCCTTTTTAGGATTTTATCATGAGTTTGAAATTTGTGTGAGGTAACGTTTTTTAT
ACTGTTAGCACATCATCTATTATTGATTTATATTACTTTTGTTTTAAAGTAATAAATGAT
TAATAAATCAATAATTAATGATTTACTGACAATGTAAAAAACACTTACATTATGAGTGTA
TACATATTAAATCATTTTATTATTACAATATTGTCAACCAAACACAATTTCTCACTAATA
ATATTGGAAATGGTTATTATTCTTTCAAGGTAAGAGTGTCTAACATGTTATTTTTGTAAC
ATTTTCCTTACTATATTCAAAATATTAAGAGACATTAATCTAGTGGTATAAGATTCTACT
AGTTCTCCAGGATTACTAAAATGTTAAGTGTTCGATTCTTGAGAGAGCAACTAAACACCC
CGAAAGAGATTAGTTTTTTGTCAAAAAAGTAAAGGAATATCATTGACAATGCTAAAAAAC
ATACTACAAATCATGTCCCAAACCAATAACAATAAAAGTAAAACATAAAAATAATAATAA
ATCTTTATTTTTTTAATTTCACATAACATATTTTTTATTTGCTTGAAATATATATACTAT
GGGAGAACAAATCCTTACTTATTAAAGATGATAAACCAATATCATCTCTAATATTGTATA
TGAACATGGTAATATGTAATATAAAATTATTTTTAACTATTCCCATTTTAAATAAATTTT
AAAATAAAAAATTATGAAATTTCCATTTATATCATATTTCTGTATTCAGCCAGGAGATAT
TCCTTAATCCTTATTCATGGTCCCTAGACCTTCAATAAAACAATAATATGTATTAAAAAT
TAACAAAATTAAATAATTTCAATTCAAATCAAATATGGATTTAAAACTTTCTTTGAAATA
TCATGCATGAAGATATGATTTAGATTTATGAGACCTAAACCAGTTAAAAAATTTCTCTTA
TATATAGAACACTCCCATTACACATTGCAATAAACAACACCTCACTATAAACA
LalbGBP1 promoter1
>prL. albus_Lalb_Chr10g0092981
SEQ ID NO: 108
TTTTTAGAAACACTTATTTATCACCGTATGAACATATATGAATATATATATATATATATA
TATATATATATATATATATAGGGTTGGGATACTCTCCAGTACTGGAATGTGAGATCCAAA
TTATTTTGAAGATTAAAAAATATTTTAATTGAAAATAAATTGAAATCTGCATCGTTAAAA
AAATTTAAACGTCACATGTTATCATCTCTGTCTGTACTACACCTCCAATATAGGATAGGA
TCCGCATTATATATATATACATATATATGCTGATGATCATTATTTTAATTTTTAATATAT
AAAATAAGTTTATATTAATAAAAATTTAATTAATTTTTTTCCCGAAACATGTGCTTATAC
ATAAACTTCCTAAAATAATGTTGGGAATATTTAAAATACACTATTCTATGAAGTCTGATA
TTACCTAAAATGAGGTGGAGGTGAGAATGCGATCTTATATATTAAGCTCAAGTTCCTTAG
TATATCTTACCCCAATTAGAAATACTCCATGACCATAGGGTTTATGTGTTGAGCTAGCGT
CGTAGGCTATATATGTGTATCCTTATTGTGTGCTTGAGCTAGTCAATCATAATCTCTAAT
AATAAATTTGTAGCAACTCAACTTTCATTAACAAATTGGTTGAACATTGTTAATAAATTC
ATTTGTCTTTTATTCTTCTATTTATTCTCTTACCCTTTTTTTGGTTGTTTGTGCTCAATA
TTGTATAACGATCGCTTCCATTGCATCAACAATTGATAACAAATAAAATTGGTAGATAGC
TTTTAACCCATACATCAATGAAAATGTTAACTTTGGATTATGGCAAAGTTGGACGAAGGA
CATGTTAGTTATATAATGCTTGTCAATGTATCCACTGAATCTGTCACCCTATTAAGGTTA
TATACTCCCTATAATATTTTATTCTCCCCTAAAGGGAGCCTACGCGACTCGAAGTCTCGA
ACCTATGACCTCACGAACTAATAGTCATTGACTTCATGGACTTAGTGGTAGCGACTTAAT
GGAATCAATGACTATAAGTCCATGAGGTCATGGGTTCAAGTCACAGATGCTCCTCTTGGA
GGAATAAAATATTATAGGGGTACTTAAGCTTGATAGGCGACATGCTCACCGAAGGGTCGC
ATGTCAAGGCTGAGGTGCTACACAAGGGTGTGTAACATAGGTGTTGGTTGACTTGGTTGA
AGGGTGCGTTGGTAGAGATGTAGCCATGACACAAGTATTGGTTGGCATTGAGGCATGGCG
TGTATGGTGGTAAGGTTGACTAAGATAGTTTCGTTAAACTATAATTGTTATTGTCTACAA
TGATTACCAGTATCGACAATGATGGCATGTATTGATATTTTCAAGTAAGTTATAAGGCTC
ATCTTTGTTGAGTATTAGAATAAGGTGGTACAAGTTCGTCAAAATACTCAGAGAGGCTTA
CACTCAAGGAGGGGTGGTCTCTCGTGTAGAGGAAGTGTCATGGAAGATCTGGTAAAGAAT
GTTATTCATTGAGCAAGGTAGAGTGAAATATTCACTAAGATGGAAATTATTGTTGTGCAA
ATTTCCTACGAAGCATAATATTTTGGATGTGGAGATTGTTATATAGATTTTCAAAAGAGT
GTGATAAGAAGCCCTAGATTGGGATGCTTGGAATAAAGTATTTAAGAAGTCCTATATTGA
CTAGAATTGGAGTGAAGGATAGATGAAGAGTATATATTAGGTTCAAGTTCCTTATTTTAT
CTTCTCGAGTTATAAACATTTTGTGCCTCACTTATGTATCGGATCAGAGTAAGACTCCTT
TCCATTAATGGAGAGGGAAGTACCCAAAAAAAGTCAAAGAAAGTAGAAGATGTAATGTCA
TTTTTGTCCTTTTTATTTTTTATGAAATTATAGTTCTACCCTCAAAAGTAACATAAAAAA
AAGGAACTTAGGATCTTTTTCCATTGGATTATGGTTGTAAGCTATAGATATGTATTATGT
AAATCATAAATGAGTGTGCTTGAAAATATTAATTCTAATCTCTAATAATATATCAGTAGC
GGCTACATATTTCTGAAGATATCAATGTAATGATTGAACATTGTTAACAAATTCTTTTAT
ATTTTTCACTTCTATTTATTCTATTTCCCTTCATTTAATTGCATGTGGTTAACATTTAAC
TCCTTGTACCAATAATTATTAATATTAGGCTTCAATATATTTTTTGTTCTTGAAAATTTA
GCAAATTTTCATTTGGTCCTTATAATTATTAGTTTTATTTGTTTTTGCACCAATGATATA
ACAACATCATAATTAGTCATACATGTCATAATTACATGTTAGCCATATCGTAGTGAATAA
AAGTAAATTTTTGAAACATATAAGATTCAAAACCAAATGAAAAAATATATATATTAAAAC
ACAAATTCCCAAAAAAATTAAGGAAAAATATATCATGGTAAACAATATCATTATAATAAT
ATAAGAGAATGGTGGAAAAAAAAGTCTGCTTAAAACAAAAGCAATTATGTACTAATAAAT
ACATATTGTCTCAAACCATGTCCTATATTTTCATTAATACGAGACATAATTTAAGAAATG
TCATTTTATTCACTGTTGTGATAACCGGATCGGACGGACTCTAGTTCATGGTGTTAACCG
AATGAATGGTTAGAACGCTTTGAACCGAATAAATTTGGGTTCGACCTTCCTAAACCAAGT
TAAACCAGAATAGTTCTGAAAGTAAACCGGACTGTTGACTTAAAAAAATTCAAAGTCTAC
TTTTTTATTTTAGTTCGATACAAAAAATACAAAGTGATATTACTTCAACATAATTACATA
AAATTATTAATATTATAAAATTTTATATTTATATAATTATATATTTATAAATAAAATGTA
TTAATCATATTTAAAACTACTTTTTGATAGGACGGTTTATCTGTTTAAACCGATTAATAT
AAAACTTATTCAGTTTGATTTCCTGTTTGTGTTCACAACCATTGCTTTTATTATCAAGAT
CCATAGG
LalbGBP1 promoter2
>prL. albus_Lalb_Chr04g0258421
SEQ ID NO: 109
TGCTTTTTGTGATTATATTATATTATATTATAAGTGATACATAATTACTTATTTCAAAAT
GAAAATTAATATCTTTCTATAAAATTAACTAACAAATCCTTTTACACCTATGATATAGTT
TCACAATATTATATTTTGTATTGACTTACCGGTATCATATATTACTAACATAATCAAATT
AATTTTGTAAGAAAACATATCAGTTCCTTTAAAATTTCTACACACAAATTTAAGTAGAAC
CTATGTAATTAACATAGGAGCATAAATCTTAAACACAAGGCTAATATTTAACTAGTTAAA
AAGCTTTGTAAAACATAATATAATAATATATTTTTATCTAATTACTTTGATATATTTTTA
TCTGACGCCAATACAACATGAATTCAATTACTGTTATGGGAGTAATCCTGACGAGCTCTA
TTGGATCATAAGATACATGTGATTAACAACATAGAATTGAATTAATCAAAAGGAATATTT
AATATCAATTGGTATTTAAACTAATAAGGGAAAACATGTTTTAACAACCAATATGAAATA
ATTTGCATTTGCATTTGTCTATATTGTTTCTCATAATAACTATATTTTTATAATTCAAAA
CCACAATTCAATTGGAACCACCAACAACGAATAATTTGCATTTTCTTATATGACCATTCA
TAGTAACTACACCTCTATAACCCAAAACAACATTTCAATTATAAATTGCAGTTGAATATA
TTCAATTGAGATAATTGTTCAATCTTAATAAAATGATACATATACAATTATGGTCATCAA
TCAATAATAGAGTGTTGCATTCTAATTTTTAAAGACTGCACAACACAATAGGAAAACAAG
TTATGAATAGTTGGTTTACTCAGCCCCACTCCTCCCTATTTAACAATCTAGAATTGATTT
TTCTTTTCCTCTATTTCCATGAAAATACAATACATGAATAGTCCTTCCATGAACAATCAA
ATAAATATTTTATCATGTTTAGTTTATTTATTACAAAAAGTTACCACATTTGAGGGTAGT
GTATCTATAGACCAAATTATATTGTTTCTTCTAATCATTATTCAATTGTTTTCCCTATAT
GTTTTACTTTTATTCCTATTCGATTATTTTATTTTCATTCCAAATATTTGATAAAAAATA
TTTTAGTGGCAATAAAAATAGCATTCCTTGTCAATAAAATAATAAGAAATAAAAAGTAGG
TATTGATAGAAGAAGAGAACTGATACCCCTAAGTTTTATAAAGAGTAGGTATTAATAAAA
AAGTAGGTATAAAAGAGAAAATAATTTTATAAATAGTAAATTACACTGACACAATCTGAT
TTTCAGTGAAATTATAAAAAACATAACTCAATTTTTAACACATACAAAAAACTCCTAATT
TATAAATATACATTACACTCACCATCTTTCATGAGAAACAAGTGTAATATTTAAAAAAAA
GTGATAAATGTAGTTAAGTTAAACAAAATATGATATATTGTGAAAACAAATCCAAATATT
ATACGAGCCAATATTTTTCATTGTTTTTGTTCTTTATAATATAATATAAGAGTTATTTAA
ATAATATTTTCTTATAATTATTGATGCATTTGGTAATGAACCAAAAATAATGAAAATGTA
TTGACTTGTATATTATTTTGACATGTCTATACAATATATCATATTTTGTTTAATTTCATT
ACACCTATCATCATTTTTATGTAAAAATCATACTTATTTCACATGTAGGGGTATTCATTA
TAATATATGTATATAATTTGAAGTTTTTTTTTATTGGTGTAAAAAATGGATGAGTGGTAG
TGTAATTTCATTATAATTAACTCATTTATAAATTTATATATAATATATTTCACTAATAGA
ATTTTGTTATTTTTATCATTAACAAGACATTTATATCCTTAAAAATAACTTAAAAAACAA
ATTATCATAATCTTTACTGTGATAATTGTATCTTATTAATAATAAATATAATTACATATA
ATAGTGAGTATAAAATCATGGGAGTGAATTCAATTACTCAACCATTAAGTTTAAAAAACT
TATTCCATATCTTAGGTATAAATTTTAAGGAAATTCAATGGTTTGAATTTGTAATCAGAT
TTAAATAATTAGATTTATATAGAATTGATGTTTAAGATGTGAAATACTCTTGAAATGAAA
TAAGTAAATCAATTCTCTAAAAATTTATGTCCCTTGTCTCAAACTAATCTCATTCTAAAG
AATATAAAAAAATTATTTATATTTTTATGAATTTTTTTTAATTAAGTATAATTTTTAATG
GAATTGATACACAATAATCTAATCTTCGTTCTGTAAAAGGAATCTCATAAAGTTAAGTTA
TTGTCGTGTAAATTGATTGGTTTGTACAAGATTGTATTCCTTTCGTAGGATAGCTTTTTG
AATTGGTCAATGAAAAAGAGTTTTATTCCTTTTTAGGATTTTATTTTGTGCGTTTCACAT
TTTCATTACAATATGGGCAGCCAAACACAGAATTCTCTCTAATATTAGAGAGAGTAGTTA
TTATTTTTGTAAGATAAAAATATCATGTTATTATAAATAAAATAATAAAAGTGTTATAAG
ATATTAATTTTATAATACCTCCTTCTCTAGTATTTGAGAATATAAACCAATATCCTTTCT
AATACAGATAATAATATACAATATAAAATTATTTTAAACTATCCATATTTTAAATTATTT
TTAAGTAAAAAATTTGAAATTTCCATTTACCTCATGATTTATGATTTAGTACTATACATA
ATTTTGAAGATGAGATGATACTTATTCATGTTTCTCAATCTTCAAAAACAATAATATATA
TTAATAATTTAAAATTAGATAATATCAATTCAAATCAAGTATGGATTTAGAACTCTATTT
TAAATATCATGCAAGAAGATATGGTTTAGATTATGACACCCAAGTCGTTGATAATTTCTC
TTATAATATATAGGAGAGTCCTATTCCACATTGCAATGAAAAACCCTTCACTACAAAAAA
VuGBP1 promoter1
>prVigun05g034200.1
SEQ ID NO: 110
AATTAATAACTTTTCTGTTTTTATCAATCATTAAAGAAATACTATTTGTGATTTAAAAAT
TATAAAAATACCTTTAATTAAAAAAAGTCAATGATATGCCTATACATTCAAAAAATGAAA
TAAACTAAAATTTGTGATTGCAAAATTGTAAAGTAAAAGCAAATGAGGAAGAATATAAAT
AAAGTCTATGAACAAATTGAATATGCTAATAAATAAGTTTAATATACGGTAATACGTTGG
CTTTTTTCTTTTTTTAAATCCGTGTACTATGGTATTAATTAAATTAAATGATAAGTGAAT
AAAAGAAGAAAATGTAGAATACGCGTATTTATTATCAAAATTTATATGTGTGACAAGTAT
GTTATGATTTTTCAATTTTCTAAATCTGATTATACCATCTATAGAGTTAAAGTTTATTTT
TACTTCTTATAATTTTCCTGTTATTTTAATGATAATTTTTAAATTTTCTTGTTAGGAGTC
TCAATTAAGGCATTGTGCTCATGGATGGTTCAAGAGTTTATAAGTTTTAAAGCAAACTTA
AAAAAAAAAATGAGATCTTTCATTTAAAATTTAATTTTTAAAGTTTTATTATGCAAAATT
ATTTATTAAATTGTCATATTTAATTTCATTTAACATCTCTTCTTCAATAAGTAATTTGAC
GACATTATTGATCTTAAATAAGCTCTTATATTTTTAGCTTTGAGAAACTTTTAACATTGT
ACTTGTGAACATTATTTTATAAGCTATATATACATTTGAAAAAGAATCTATTATTTTTAT
ATAGTTAAGAATGTCAATTAGTTTATCATTTTCTAAACCTATAATTTCTCTTAAGATATT
TAATTCTGAAAATAAATCAAATTAATCAATATCCAATAAATTATCATGTTTTAAAGATTT
TTTTAAGGTTTAATCATTTTTATTTAAAAAATGTACAATCTAGTGACTTTAATTTTTTAG
CACTAAATAAAAAACATAAATATTTTGATATGTTTTAAATTGTTCAAATCTACTTTGAAG
TGCGGTAATTGTTTTATCTTCAATATTCAAAAGGTAATTGTTTTATCTTTTCAACTCAAC
AGTAAGTTTTTTTCCCTAAACTTAAAATATCTTTCTAAAAAAAACCCAACTTTGAATTAT
GATTTAATTAATATACTATCCACCAAAATACTAACCCGTAATCTAAATAATAGTTTTGTT
GAAAAATTTTAATTTCTCGTCACGATACATACAAGAAAAAAAAAACTCTCAAACTCATAA
AACAAAAAAGTATTGAAAAATAAAACTGGACAAAAGGAACACATTTATAATTTTAATATA
AATCCAAAACTGACTAAAAACAACACTAACTAACAAATAGAAAGAAAAAATATAATTTTA
TAATTAATTTAATAGTAGTATATTATTACAATAAAAATTGTGAGGCGTTTTTTCTTAAAT
ATAATTTTATAATTAATTTAATAAAAAGTATTAATAGAAAAAAAAAATTGAAACCTTATT
TATTTGGAGGTCTAAAGTGAATATATGTGAGATACAATTTCTAAACAAGTTTTTTTTTAA
TAACACAAACTTTTGATTGAACAGGTATAATGTTTTAGTTAAAATAGTAGATTTTTTTTG
TTAAATTCATTTGTAAACCAATGTATCAATTATAACACATAAAAAGGTTGAAATAAATAT
TATCCTATCGGTAATATTAAATTTTTATGATATACATAATCGTAAATTGATATGTAAAAC
AATAATTCTAAATTAAATTTGTTTTCAAAACAAATTATTGTGTGGATTTGGATCTCTCTG
ATCCAAAATAATTCACATTAGAAAAATAATCTATGTAAAGGAATCTCATTTATATTTAAG
AGTAAAAGTTTAATAAATCAATCAAGTTTAATCTTATCATACAGTTCATTTCTGTTCATC
TCAATAAATCACATTTATTAATTTGTTCCTGTCCATTCCTTCACTCTCTTTTTTTGTTGA
CCTTTATACCAATAAATGATTGTATAAACATTGCACATTACATTTTCTATTAATCCCAAT
TTGTTTTCTTTTTTAAAGATTTGATTTAATAATAAACATTGCACATTACATTTTTTATTA
ATTAACCACAATTTATTTTCTTTTTGAAAGGTTTGATTTAATAATAAACATTGCACATTA
CATTTTTTATTAACCACAATTTATTTTCTTTTTGAAAGATTTGATTTAATAATAAACATT
TCACAATACATTTTCTATTAACCACAATTTGTTTTCTTTTCAAAAGGTTTCATTTAATAA
TAAACATTGCACATTACATTTTTTTATTAACCACATTTTCTTTTCTTTTTGAAAGGTATG
ATTTAATAATAAACATTGCACATTACATTTTTTTTATTAACCACAATTTGTTTTCTTTTC
TAAAGGTTTGATTTAATAATAAACATTGCACATTAAATTTTACTGACCACAATTTGTTTT
CCTTTTCCTTTTTGAAAGGTTTCATTTAATAATAAACATTGCACATTACATTTTCTATTA
ACCACAGTTTGTTCTCTTTTTGAAAATTTTTATTTAATAATAAAATGTTAAATATGTTTT
TTATCTCTTAACTTTTAATAAAATTTGAAATTAGTAAATTTTGGACTAATTTAGTCTTCT
AACTGTAGAAGTGTATAAATTTAGTTATTTTAACCACATTTTATTAAGTTTATTTAAGGT
TTCAAATATGTTTCATGATATTATTTCAACTAACATTGAGGTATGAGGATATGTCAAACG
GTATAAACAATTTAAATACTATTACAAATGTATTTAAAACATGAAATAAAATTAACAAAA
TTTGGTTAAAATGACTAAATTCATGTATTTTTAAAGATAAATGACTAAATTAAGTCAAAA
TTTTTAAAATGAACTGATTCAAATTTTCATTAAAAATTTAGAGATATGAGAAACATTTTA
AGCCAATAATAAACATTGTACGTTACAATTTTTTATTGAGATTCTAAAGTCAATCTTCAT
GCTCTATATATATGTAGGAGGCAACACTCAATATTGCATAAGGAACGATCAATCCCTTGC
TCTTCCATACACA
VuGBP1 promoter2
>prVigun05g034300.1
SEQ ID NO: 111
TGAATTATTTTCGAGTGTTTTTATCATATCTAAGGCTTTTAATATATGGTCTATAACAAC
GATTCCAGTCACAATTTTATCTTAGATTATTGAGCTATCACACAAGAGAAAAGAGATTAT
GTTGAAGACTAAAAAAATAAAAATATTTACTTTAATTTTACGATTTATATGAAGGCATCT
TTAGAGAAATTTAAAAGATTATTTGAATTTTTTTATTAACAATGTAAAGAATTTTTTTGT
ATCAAAAAGTTTGAACCAGTTTCGAATTTTAAGATTAAGGTAATATGTTACGGGGATAAG
ATGAGAAAGGATTGTGTATATTGTAGGAAAATTTTTGAATTTTTTTTCTATATTTGATGA
CATTGTTAAGAAGTTATTTCTCGAGTTAGGAAAGATGGTATAATATATCCATTTTTATAA
TTAAAATTCTGGAACTATTACTATTATAAGTAAAGTTAAGTCGCAATTCGTTGTCGTCAC
AAATATATCATCAATATTTTCTTTTTTTTAATACATAACTTTTAGTTATAAAATAGGTAG
GTCCTCTTCATAAAATATTTTCAATAATATGTTTTACTTTTTTAAATCACTTGATTAATT
AATTCATTTAATATATTTTTTAATGGCCAAATATGTTTGAATATTTCTTCAGTATCAACA
TAAATAAAATAATTTCTAATTACTTCGTGAAAAAAATATTTTATGTGAAAAAAGAATCTT
AATGTCTATGTTTTTCTGAAAAAATAATTTATATTTAAATTCATGAAATTAAACAATTAA
AAAAATTTAAAGTTGTTTTAGTTTATACAGTTTGAAAAATACTTATATAAAAGTATTGTC
AAGAGATTAAATATAACTTAAATTCGATTATGTAATAGTTTAAAACTAACACGATAACAT
ATGTGTTCTCATGTGTTATGTTTTATGAAAGTAGAGTTGGATGGTAATTTATGTGGCTCA
ATCAATTAACAGAAATTCTTAGAACATGGCACGTTTAAACATTAATCAAAGGCATTCAAA
AAAGGGAAGAAAAACAAAAGACATTAATGAAGAATTTTGGTTCCACCGTAAGAATTGGAC
ACATGCTCTGCCTCGTAAGAGTAGGAAGCTCATGCTATAAAATATAATCAATCATTCACA
CTCTCCAACTTACTCAACACCAAAGAAATCCAAAATTAATAAAGCTAGGTAAACCTCAAT
TATTATTCAAAAGAAATAAACAATATTTTTTATTAAATATACTTTTTAATTATCTTACAT
CGTTTAATATTTAAAAAAAATTGAATTCAATTGATCTTTGATCTTTGGTTACATTATCAT
ATAATGTTTAATTTTACGAGCAGGCTATTTCTTTTAAATATTAAAAGATTACATTATCAC
ATTAGTTTTTTATGCGTATAAATGAGAATGGAAGTACTACTTCCGAAGTAGTAATATTTT
TCGTTTTATATCAATGGATCGTCTATTTAATTTTCTTGTGTTCCAAAAATATAAATTGGA
TCGTTTGTGTAGTACTTTGGCTTCACCACATATGTCAATTTCCAGTCATGTGTTGCCTAT
TGTACTTTATTTTTTCCTCTGTTTTTCATTCTTGTAAAGTTATTTTTAAAATTTGAGATT
GAGTGTAGTTTATCTCCACACTAATTATAGCTTGTAAATGTTTAACACTTTACATGTCCA
CACGAAAATTTGTATTTAGGATCGAATAAGATTAATAAACAATCTTCATCTTACGAATTT
TGTAAAATTAGATTACATTGATAATGTTAGAATATCTTTTATAAATTATTTGTTGTTGGG
TCTATTAAATTACTTTTTACTAGTTCCAAATTGAATATTAAAATGTTATTTATCCATTTG
CAAATGTTTAATTTTGCAATCTCTTAACTCTCGTGGGTGTTTGAGATCAACTTTGACTAG
TATACGAGAAACATAATGCTTTTAAGAGAGAGATAATAATTATTTAACAAACCAATGGAT
TAAATTAGGTTCAAAAGTGAATTCTAAGATGATGCTATATTTTATCTTATTAATTTAATA
GACCTCTTTATTGAATTATCAACAATCTGCCTACAAAATTTTAGTCTTTGCAAACTTTGT
AAATATTTAATCGTGTAAACAAATGTGATAATGCTTATCCTATATTTAAAAAAACAAATT
TAATGCTTACAATCTATTTATTTAACAAAACAAAGTTTTTATGTCGTTTGTATATTTTTT
AATGGAAGATAATTACCAAGAGAATATGACAACAAGTATATATAAATTAGAAGAGTGTTC
CTTCACTATTACACAGGAAGAATTATGCATTACTTAATTAATTCATAATTAATTAGTTAG
GGTGTACTTATCAAGGAATGGAAATAAAAAAGATGTAACTTGTGATTTAATAAATAAAAA
GAGAGTCTAAAAAGAAATATAAGTTCGTTAACCAAACGTGTGGGGGGAACATATAATATG
CAAGAAGCAATATTATTCAAAGAAAAACAAAATACGCTTCGTACGTACGTACCTTGAAAG
AAAAATTATGCAAATAACAAATGATACGAGTTATGGAAAATATAAACTATGTCAGACTAA
AAAGTAAACCAAAATCATAATCTGTTCTATTAAGCCCAATAATGCCTTTATTATATGGAA
ACTGATATTTTGACATTTTTGGATTTCGTTACAACTACTTAGTGATTTTTTTATAATAGT
ATAATAATTATATTATTATATATTCTTACAACAAGTTATGAAGTAGTTGTACTTAAACTT
AAACATACACGTTCTTGTACACGTTGTATCATTGTCTCAAATTAAAAGGAAAAAAAACAC
AAAAATTTAGCTATAAATATCAACATTTTTTTCATAATGATTAAAAGAATAAGAAAAAAT
AGTAACACAAGTGTTTTTTTTTTTAATGAAACTAATTTAATAATAAACAGTGTACAGTGG
CAGCCGGGCAAAATTATAAATTTATAGATTAGAATCTCTTGGTTCTAAAAGTCAACCATT
AAGCCAAGAAAACTCTTATCTTTGCTTATAAAGTGGCTGAGGCCGCGGCACCACACAACG
CTGCAAGAAGCAATCACAAACAACA
VuGBP1 promoter3
>prVigun05g034000.1
SEQ ID NO: 112
AATTGAAAAAATAGTGGTAGAAATAATTCAAATTTATAGCTTCTTTCATGATATATTTTT
TCATCAAATGATAGTTTTACTGCTACAAGTGAATATAAATTTCTTACTCTAATACCTCTA
ATAATATTAAACTTTAATCATTATATGTATTTCAGGTATTATTTATTTTGAGTTTAAAAA
TGGAATTAAAATATATCTAAACTATTTTTATCTTTATTTTTAACTAATATGACACTATTT
AGAATATCAGAACAATTTTTTTCCAATCAAATGTTAAAGGAATTTTTATACATATAGCCT
TGATATTTTAACTTTGAGTTTATTGTTGCATATGAGTATTATCCACTTTAAAATTTAGAA
CTCCTGATACAATTTTCTTTTTAAGGAAGGTTTTAGAAAAGTAAAGAAAAATATATATTT
AAATTTTCTTTAACATTATTACTAAAATGAGAGATATCTATAAAATTAATTGGTAAACAA
AATTTATTGGAGGTAGGAGTTTAAAAATAAGTCTTGTATTCTTAAAATTTCATAAAAATT
ATATAAAATATGTAGAAATATTAAATATTGTTTCCCTAATTTGCAGGGAGTTTTGCAAGA
TCATGCTCTGATTCACTCTTTTTAAAATATTGCTCTTCTATGGAGACCAATTCCTTCAAT
AAAACCTATTCATATATCAGCCTTTCAATTTTTATTATTTTTTTTGCACACGTACTCTTG
TTTAAAATAACAAAATTCAATCTTGAGTGACACTAACTACAAATAAAGAACACTTCCTCA
AAAAACACTTAATCAAACAACAAAATCCATTCACACTTTACATTAAATAATGGTATTTTA
AGAAAATTATAATTTTACACTTATTACATGCTCCTCAATTTATTTTTTATTTTTGTCTAG
ATCACAAGTATAATTTATTCAATATGGTTAAGGTCCCAACAAGTAGTAAATATCTGCTGA
ATAAAATGCAATCATATATTCAAACTTAATTTCACTTAGTAAGTTGCATAGTAACTAAGT
ACTTTTTAATGTATAATAACCTTAAATATATTTATTAGAAATCTAAGATAAATTAGAAGT
ATCGCATTAAGTACAATATAAGTACTGTGAATAAAACATAAGTAAAACACCTACAAACTT
ATTATCCATGTAATGTATTTTTAGAAACTCAAACATTATGAGAATTTGAAAAGAAAGATA
TAAATTATAATTATAAATATAAAACACTACACACAAGAAGAATCAAATTGTAAAGTACAT
AGGTTTTGGCTTTCACTTTGAAAAAGACAAAACAATGAATGGTATTTTGCTTTCAGCATA
TGGCCACTGTGCTACACATGGATTCGACATTCAAACACTACAAAGCCAAACATAGATGTC
CAACGGCAATATTCTCAATAATTGCTAAACTTCTAATACTATTTTTAACTTAACACGATA
ATCAATTATGTGATGTGTTATACTTAGAATAGTTATGTAAGAATGAGTCATTCTGAATCC
GCTAAAATTTTCTTCCTGCTACTTTTCTTAATTAGTATAGTTCACAAATAAATATATTAA
ACATTTTATGTTTTCAAGATATTAAATATCTTTTATAGATATCAAGACAATTTATACAAG
AATATAACATTATTCCACTATAAGAGTTTGTCGTGCGATCCATAAACAAAAACTCTTAAA
ATATGCCATATTTGAATATTAAAGAATAATGCATTGAAAAGTGGAGAAAGAGAGAGAAGT
TTAACAAAGCAAAGAAAAAGTTTCCGTTCGGCTTGGAACTTGTACAATCCTTGGAATCGA
TCTTCCATGTCGTCCAACTAAACTGCCAGCAAACACAAACCAACAAATGAAAACTATTAT
AAGTTTCAGAATTAAATAACTTTTTTACACTTCATAACTTTACTAACTTTGCTTCTTGTT
TTTTCGCACAACCATTTAACAACAACCCTTATTTGACGATTTTTATATTAAGTGACACAA
GAATCTCATTAACATGTTAACTTTTTTCCTGTGGTTCAATGAACGTCTTAATTAATTAAT
AACTAATTAATTAATTAATTAATGTAATATGTCCGTTAGTTATTTTTTAGATTAAATATA
TTTATGGTCCTTTAATTTTTAGTTAAAATGAAATTAGTTATTTTTAAAAATTTTGTCAAT
TTAAAAAAATGTGTAAAGTTGATTATTTTAATCAAATGTTAAAGAAATATTTAAGTTTCA
AACATGTTTCAACTGATATTTAAATTATTTCAAATACATTTAAATTTATATGACATTTCT
AAATTAACATTAATTCAGAAATGGTAGCACTAACCGCGTTTGAAATATTTTCATAAAAGT
TGAAGTTGATTAAATAAGATTAAATACACTCATTTTTAGAAATTGAAAAATTAAATTAGA
CAAAAATTTCGAGAAAGACTAATTTTAATTTAAAAAAAAAACATATTTAATTCAATAAAA
TAACAAAAAATTCATCACTTAATTGTTTTAAGAAATACTAGAAATCAAAACAAAAGTAAA
AGTTATATATGAAAACAAAAACAGAACAAAAATCATAAAAAAAAAAGATAGAAAAAAGTT
ATTGGAAAAATATGTCTTATTCAATCATTAACACATCACTTAATTATTAGAAAGCGATAT
AAATGAAAATAAAAATAAGTTTTTTTGTTATAGCACAAAATGATAATTTATTTTATCGAA
GTCATATACAAATTCTGCATTTTTGTAAGAGAGAAAGAGTAATTTAAGCTTGAATTATAA
CGTATAATTTACCCAGTCAACAGCTTCCAATTACGTCACATCTGGGCTGGGAGTATACGA
AGAGTGTGAGAAGGTAAATGTTGTTTTCCCATTACAAAACCAAACACAAGGATTGTGAGG
CAATAAGTGTAATAATTATTCTAAAGTCAACATGGCAGGTCAAAGAAACTCTTAGCTTGG
TTCATATAAGAGAGCACAAACATTGCAGCAAGCAAACACATCTCTTCCTCATCACAAACA
PvGBP1 promoter1
>prP. vulgaris v2.1|Phvul.008G033200.1
SEQ ID NO: 113
TCTCTGTTTGTGCTCATATCTGTTCTTCGTTTTCTCTGGTTGCGAAGTTCTGCATTTTCA
AAGGTACGTAACTGCATTGCCCCTCATTCTCTTGTTTCCATTGCCCCTAACAACTGCATT
GAACTCCAATGGCTTTCGCTTTCCATATTGCTTTCGTTTTTAATGGGTTTCGCTCTACAT
TTTGAAGGTGTATTGTTCCATTGCTTCCGTCGCTGTCGCATTTCGCTCTCGCCGCTCCGC
CGCCGTTGCCGTGTTTCCTCTTCTCCCCTTCCACTCTTCACCAACAACGTGCCCCTAAAA
CCCTAGCTCATTCAGAAGAATATACAAATGCGGCTATTTCAAATAACCGCATTCGTAAAT
CGCATTTACAAATGTGGCTCTATAGCCGCATTTGTAAATGTAAAATAGCCGCATTTGATT
AGCATTTTTGCGCTAGTGCACCCAATTATCTTTAGTATCATACCATTGTGACTTAGTTTC
CACACTTATCTTGCTTGCCATAGATGAATCTTCAAACTCCATTGGTTCATACCCTATGAT
CTATCATTTGATACAATTTGGTCATCATCCTCCATGGTGATAACTCCACTAAAGCTTTTT
TTCTATGAGAAACTTGATATAGGATTGATGTCCTAATGAATACCCCTCAACTCTAATTTT
ATGGGGAATTTATTGTGTCCTTCTATATAAAAGAATGTACATCGTATTCTTACCACTTTT
TTTATGCATTCAACACTCTTGCTAAAAAAAAGGAATATCATAAGGCTAATCACATACTAA
TTTATTACCTAATAACAATTATGAGTTTTACTTGTAAATATTATTGAACATGCCCCCAAT
TATACCCCTTTACTAAAAAACATGTCATTTATCTTATTCTCTTGTCCAAAAATCACAAGT
TGGATTGTCCTTAAAATAAGATAATATAATTCCTCTCATTTTCCACAAATCCACTCCAAT
ACTTGGTAGTTTAATTTATCAAACATTGTTAGGTCTTTGATTCTTCTCGTCCAACTTTCT
GTAAGTCATTTTTCTTCTTCCATCTCTGAATTTATTATCGTATCATTACATTTCTCAACA
TGGTCTTGAAGTCTCATCCTTGACTTCCATTTTTGTGTAGTAGCTTTTGACTTCACCACT
TTCACATATGCAAGTTCGTCAATCATGTGTTGTCTTCCATGTTCCATTATACTTTGCTTT
TTCCCCTTATTTTGGCGCATATGAAGTTGTTGTCTTTCAATTTGTATTATTTGTACGCTT
TTATCCTTAAGGATGTTAAAAAAATTCACATTCTCAATCATGAGATAAAGTTCTTTTTCC
ATTTTCCTTTCATCTCTCACAACCTTAAACCTAAAAAATTCAAATCTTTGTCCTACTTTA
TTCTTTCACCTAGATATGAAGATCTCTCAAACCTCATCTCAAGAATCTTCCACAAATCTT
CTTCATTGTAAGCGCCTACGAACCTATAAAAGAAGAAGGTGGACATGTTTTCTCAAACTC
ACCATATCTCAAAAATATTTTTCATGAATCTTTTCACTCTAAGCACTTGAGAACTTATAA
AACAAGAAGGTAAAGATGTTGTTTCAGTAATTCTCTCAACTTTTCTTTTCGCTCGACCTC
GCTCATAATCGATTACTCCCCACATTTTTATTATCTTATTTATAATTAATGTGAAATATT
TAAAAAAATAGTATATCCCATCATATCCTTAATATTAGTGGATTTACCATGTGAATAACA
TAATAAGTGATTTAAAATATATTTAAATATTTTCTTAAAATTTGAGATTGAATTTAGTTT
AACTCCATAATAATAACATGTAAAATGTTTAATACTCTATATCTAGTTATCTTCATGAGA
ATTTGTATTTAAAATCTTCATCTTATAAATTTATGTGAAATTTAATTAAATTTAAATTCA
TGTGTTGAGATAATGTTAAAATCTTTTCTATACATTGTTTATTGTTGTTCAAATTACATA
CAAATGTTTAATTTTGCAATCACTATGTCGATAAATCTTATCCTGAATTTGAGAGAGGGT
ATTACATATCCAACTTAGACTGCTATATAACAAATATATTACTTACAAGATAATAATTAT
ATATCTAATAAACTAATGGATTGAATGAGATTTAAACTTGATCTCTAAGATTTCTTTATC
TTACTAATTATTTGAAACCTCTTTATTGATTCATCAATAATCTACCCACAAAATTGTAGT
CTTTGTAAACTTGACAAATTTTTAATCGTGTTAAAAAATGTGATAATGTGCTTATCATAT
GCTAATGTCAAACTTGACCTATACTTTATATATGATTATCATACATCTAGTTGTTCTTTC
ATCTTCTTTATTATATCTATTAATTGTTTTTCTCTCTTTATCTTGCTTGTTTCCACTTAT
AGACTCTAAAATGACATTCAGCATTAGTAACGATTAAAAGAATAAGAAATAATAAGATCA
ACACATTATTTTATTTTTAATGAAACTATTTTAATAATAAACACTGCACAGTGGCAGCCG
GGCAAGATTATAAATTTGGGAAGTGTTGTGTTAACTTAACACTTCAAGATTTATTATCCC
TTCTTGCAACTACTTTTATTAATATTTCAATTAAATAACCTAATTCAATATTTTATTAGT
ATATAGAATTATTAAATGGTTGTAAATGCATGTTTGAAATAGTTTTTTTCTCTAAGGGAA
AAAATATTTTAACACCAATTTTTATAAATAATTTCATATCAAAACTGCGATTAAAATAAT
AAATTAATAAATTAAGTTAAAAGTATTTCTTTCTTAACGTTCATCTTGTAATTGTATATA
TTACTATTTTATTGGAATACAGTATTTTATTAGAAAAATCAGTTAAAAGTAATGTATTTA
ATGAATAAGATATTTTTCCTTAACTAAGACATAAATTAAATTTTAATAAATAGTTATTAT
TAAGAGTTGTTAGGAAGTGGTAGAAGTTTTAAGTATCACTCTTCTTTTCTTCATTATACC
TTAGAATTTCTCGTTTATAAAAGTCAACCAATATGCCAAACAAACTCTTATCTCTTTGCT
TATAAAGTGGCTTAGGCTGCGGCACCACACAACACTGCAAGAAGCATTCACAAACAACA
PvGBP1 promoter2
>prP. vulgaris v2.1|Phvul.008G033100.1
SEQ ID NO: 114
GATTGTAGTTTGATTGAATTTGGAAGTGTTGTGATTGAGAATTGGTTGTTTGATTTAAAT
GGTTTTGGAATTAGAAATCATATAAATGTATAAATGGACATAATAACTCAATGATTCAAT
GAGAGTGATTGTATCATGTTGAACAAGAACTGTCAATGTAATGTGTAGATTTATAATTTA
TGAATTAAATGAGGTATTGATTTATAATCTAATAGGTATATTAAGTTATGCAGAATTCTG
TATATTTCACTCAAGCTAGCAAGTTCTAGCTCAAGCTAAAAAATTATGGGTGCTCTCTGG
TGGATTTTAGCTCAAGCTAGCGAATTCTAGCTCAAGCTAAAAACTTATGGGTGCTCTCTG
GAGGATTTTAGCTCAAGCTAGCGAATTTTAGCTTAAGCTAAAATTCTGGGTGTTCTCTGG
AGGATTTTAGCCCAAGCTAGCAACTCAAGCTAAAATTCTTGGTGCTCTCTAGAGGATTTT
AGCCCAAACTAGCGAATTTTAGCTCAAGCTAAAATTCTGGGTGCTCTCTGGTGGATTTTA
GCTCAAGCTAGCGAATTTTAGCTCAAGCTAGCGAATTTTAGCTCAAGCTAAAATTCAGGT
TGCTCTCTGGCGAATTTCTAGCTCAAGCTAGAGTTTAATAATAATAATAATAAATAAATA
AATGAAAATAAAAATATTAAACTAATTTTTATTTGCTTTTAAAAGATTGATTTATTTAAT
TCTATATAATTTTTCTTATAAAGTATGCAAACTTTTATTTCATGTATAGTTAATTGAAAA
TCTCTTTAATTAGACATTTATATGATTAGTTAATGAATTGTTGTGTATTTTGGATATTCT
AAACATTGGTATGATTTGATTGTTTGATGCTGGATATTATGAGGTTCAAAATATGAATTT
TAATCAATACATAGTATTTTCAGGAAAGAAAATACCGTGTTAAGATTATTTAGGATGTGC
ATTGACTAGGGATTCGTCTAGAAGGAGATATCCTGACTCTACTGAAATAATGGAATCATA
TAGATGAAGTTATTAAGGTGGTGAAAGTCGAAGGAGGTTCATATGAATGGGTAAGTTGTT
TGAAAGATAACTAACTTGACCTGTTATATGAGTTAACCCTGTCATACTAGGAGAGAGATG
ATATTGAGATTAATTATGCGCATAGTTGTGTGGATTTCACAGTGATGTAGTATGACAGGT
GCAGATCTTTGAGTCTAAGTCAACGCACGAGTCTTCAAGAAGTACGAGTTAAGTGTCTAT
GTGTTATGAGTCAGTAGAAGTCTGACATGAGAAAGATGAATATTGAAATTGTTGATAGAC
ACTTGATGGTTATGTGGAATGCATGAGAAATGATGGATTTATGTGGATTTTATAGTTATA
TTATGTTATAAGTTTTAAAAATACCTAGCTTACCCTTTGTTTTGTTTTGTGGTTGTTTTT
CTTTGATCTGTGATGATCGTGTATTTTACACGAGAGCAGATGATATTACAGGTGATCAAG
TTTTCTCAGTGAGAAGATGAATGATGAAAAATGTTTATTTTCTTTTGAAATTTTGTTTTT
AATTCTTTTATGTAAATATTTCCAGTCTTATAAAGAGAGATAATTTGAAACATAATATGA
AAGAATTGTAAATATATTTGTATTATTATATATTTTATTTAATATTTAATACAAATTATA
ATTATTAGATATGAGAAAATATAGGAGGTTACACTATTTATAATGTATTAAATATTAGTT
AATAATTTTTTTGTTTATGTTGTTTTAACTATGTATTACCCACTCCCACAATAGTTTCTT
GATTAAATTCACTCAACAATTACTTAGTGATGGTGTAACTCAACATATTTCAATATCTTG
AAATAATATTGTCAAGATTCTTAAAGATTTCCAATTTTCTCAATATATACTCTCCACTCA
CGAGCTTTATTCTATGTTCTGAGTGATTTTTAACTTTACAGTTTGAATCATTATCTAATT
TGTTAAGTTTGATTTTAATTTCTATAAAAATCAAAAGTTTATTTTTATTTCTTATAATGT
CCATGTTATTTTAATTATAGTCTCTTTAAATTATTTTTATTTCAATTTAATGTTATCCAT
TTTAACCTTAAACAATGTACACATTTTCATACTCTTTTTAATAATTTATTCCTTTCCTTT
TTATTAATTTATTTATTTGTTTATTAATTTTATTTTATAATTTATCAATTAAATTATTTA
ATAATTCATTGAATTGATGAATGCACAATATTAATTTATTTAGAAAAAATTAAGCTTCTT
TAAAAAAAAAGTACGTATAGGAATTTGGTGGAAGGACCGAATTTTAAATTGGTTTGTAAA
ACAAATTTTTGTATGGATTTAGATGGTAAAATGGACTTTTGGCCCGCTAACCTGCCAAAT
TTTGATATGCTGTTTTTAACCTTCTAACATAACTTGTCTCATCTAACTCGTCAAATTGAT
AGGACACCACTAGCTTATTTTTAAAATTAAAAATAAAATAAAATATTTTTTATACTATAT
TATTTAAAGAATGCAAATTTATAAAAACAATATAACTTAAATCATAAACACTTCTATAAA
TTAATTTTAATTATTAATTATAACAGATTACTTTAAATGATTATTTTAATTTATCCTCAA
TTAAAGTTTTTAAAATATTTATATAATTAAATATTTTTTAATATGTATATATAACAAAAT
TTAAAAATACAAAAACTATCTCAGTATTAATCTCATTTATTAATCTGTTCCTTTCCTTTG
TTAATTTTGTATGTCTATATATATATATATATATATATATATATAGACATTTGTTCTCCT
CTATACACCACCATTTGTTTTCTTTTTTATAAGTGCAATTTAGTAATAAATATGGAAAAT
TACATTTTCTATTTATATATAGCAATATTTATTGAGATTCTAAATTCAATCTTCATGTTC
TAAACAATCTCTTGTATATTAAGGAGGCAACACTGAACATTGCATAAGGATCAATCATTC
CCTTGCTCTTCCATACACA
PvGBP1 promoter3
>prP. vulgaris v2.1|Phvul.008G033000.1
SEQ ID NO: 115
TTTAGTTCACAAAAATGTCATACATTCAAAGAACACATGGGAGAAGATAGCATTGGAGCA
TAGATACCACAACAAAATTATTTAACTCACTCACCAAACACATGGCCCCTATATTTGTAG
CATAGTTGCTTCTTGTAGGTATTATACATGAACTATTATTTTTGGCTTTTGGGTCATACA
TGGCTGCTCAAATTATTCACAAGCTTGACTTATGCGTCAATTCCAGGATCTGGACAGCTT
ATACAAAATTTCATAATTTAATACTTTTTTTAAAGATTAATTTCTACATTTTTAAAAGAC
TAGACAACAATTACGTTAAATTAATGAAGTAAAACATATTCCTGTGATACAATCAAGCTT
AAAGTTACTAATATTTTTGGTGATACAATCAAGCTTAAAGTTACTAATATTTTTGGTGAA
AGAAATACGTGTGTTGATAAGTTGGTTAATTTATGATTTATTAATAGAGAATCATTTCAT
TGGTATAATAGACTTCCATCTAGTATGTTCTTAGAATTCTTTATGGATAAGTATAGTCTA
CCTATGTATCGTTTTTGTTAACGTATGAGTTTTGGTCTAGTCCCCCTCATATTTTTTTAT
TTTTTTATTTTCTTTTTTTTTTTAATAATATTTTTTTCATGTGATGACAGATGATTGTTG
TTACTTGAGGTGTCAGCCTTGGTAAGATGTCAAGTTGTATAATGATGCCTAACATGAAAA
TTTTTTATAAAAAAAAATAAAATATTTCAATAACTTGTGATTATTTTTCAATAACTTTTT
TCTGCATTAGCTTTATAGATACTCTTTTTATATACAAAAACCAACTTTTTCATATTGAAA
TAAATTAATTATCTTGTAATTTTTTTTACTGAAGATAAACGAGTATTAACGGATTATAAT
TAGTTATAATAATTCATTTTTACAGCGTAATAATGACATATACTATGTATATAGTGTTCT
AATTGTAAAAGCGTTAGTATGTGGAAATGCTTTAAAACTATTAAAATAAAAAATAATTGA
CACAATTGTTTATATGTAATTTTTTTTTACCGAAATGTTGTTGAAGAAAGGAAATGCTGT
AGAAAAAAATATACACAATGAATTACTGAAACAAATTAACTTTTACTAATATACCAAACA
AAACCTAAAGAAAGAAAAGAACACTCTTTGCAAGTGCAAAGCACGTTATTGAAAAAGCAA
GACTTTGTTGCTTTTATTGAAACGTCAGTATTGACTAAAACTGAAAATCAAACATATTTC
AAAGCAATTGCAGGGAATTTAAGAAAAACTATTTTTTAAAAAATTTAAAATAATAGTTTA
TTTATTAAAAAAACATTGTTTATTATTTTTTAAAGTGTTTCTTTTGAAAAAAAATATAAT
TGTTTTACTTTAAACTATTTATTAACAAAAAAATTAATTAGTTAATTAATTCAGGGATTT
TGTTTGATAAATAATAATAAAGAAAGTTCAACGGTGGTTTTTTATTTTGTAGTTTGCTTT
AGAATATGTAAAAGCACTTTTGTCTGTGAGTTGAACACATTTTTGTTTGTTGTTTAACGG
TCACCTGCTACGTCTTCTATAAGGGAGCGTTGTAACGTAAACCATCGATATTTGATTTCG
TACGGACTACGTTTTTTATTTTTCATTTTTAATTGAATATCTCTTTTAATTTAATATTTC
AAAAAATAAATTTTCAACATTCCCTCTGATTTTTTCTTAATTTCTTAACCAGTGTTTTAA
GACACTACTCAACCCGATTCATAATATTATATTATAATTTAACAATTAATGAAGAGTTGA
AGATTATTGGAATAATTTACATTATATTAGTTCAACGGTCATATACATCATGCTGCATCT
TACTTACAACAACTACTTATTTATTTCTCTCTACACAATTTTAATTTGATTTTTACTTAA
ATAATTGTTTATAGATTTTCAAAGAAGAATACATTTAAATATATTTTTGCTAACTTGAAT
TTTTTTATTTTCGTCTAGAATTTTTTTTCTAGTCCTCACATTCAATATAATTATTGAGTT
TTTTTTTTTATCCTTCAATATATCCTCAAGTCGATCTAAATATACATAATTTTTGCAATT
TCTAAACACTAGAAATAAAATAAACACTGAAATAGCAACATTAAATCTACAACTCCAGAA
AAAAAAAATACTTAACCCTAGCTAATGAACAAATTTTTTTATAAGAACAACTTAATAAGA
TGATCAGATAAAAGTTCACAATGATTAGAAGAAGAAAAAGTAATTCAATGACACTTTTAG
CTGATATCAATAGTTAATTATGAGTAACCAATTATATTATGGGTTATACTTTAAAATAAT
AGTTAGATAAGAATAAGTCTTTCTAAACCCTCTAACAAGTTCTTCCTGCTACTTTTCTTT
AATTGGTATAGTAGATAAATAAATATATTGAGCCATTTATGTTTTCTTAAGATGTTAAAT
ATTTTTTTTATCGAATATTGTTTATAAATATTAAGATGATTTATACAAGAATATATCATT
GAATATTAAACAATACATTGAAAACTACAGAGAGAAAAAGAGGTTTAACAAAGCGAAGAA
AAAAGTTCCTCAATTTGAATAAGCTTGGAATCCATCATCCATTATTTTCCAACCAAACCG
CTGCCAAACACAAACCAACAAATGAAAAAAATACACAACTATTTATAATTTATAGAATGA
ATGTTTACACTTTATAGAATTATTAACTCTGCTTTTCATTTTTAATATTTTTGACTGAAG
ATTTTTCTATTAAGTGGCATCAGAATATAATTAACATGTTCATTTTTTTTCTTTTGGTTC
AATAAATTTTATATGTGTGTGTATATTACACATCACTTAATTATTATTGTATTGACAGTC
ATATACAAATTCTGCAGTTTTATAAGAGAGAAAAAAACTATTTAAGCTTAAATTATAAAT
GTATAATTTACCTACTCAATACTTAATTACATAGCTTTGAAGTGTGAGAAGATAAATGTG
GTTTTACCATTGCTTAACCAAACACAAGGATTAAAAAGCATTATTCTAAAGTCAAGTTGG
GAGGTGAAAGAAGCTTAGTTTGGTTTATATAAGAGACAACAAGCATTGCAGGAAGCA
GmGBP1 promoter1
>prG. max Wm82.a2.v1|Glyma.08G245600.1
SEQ ID NO: 116
GATGACAATATTATTCTTTCTTTTATATTGATTTGGTTTTGATTTATTTTGAGAAATTAA
TGTTAATTATAATTAATTTTTGTCTATGAGACAAACTTGCACCATATGAAATTGACAACT
CTTCTAAAAAGAAAAAAAAAACTTTAATTTTGAGACATATTTTTGTATCAGCCTTTAATT
TAAGACATACTAATTAATAACTATTTTTCTTTTTTATTACTATTATTCATTTTTTCGCTT
ATCCTATTTTTATTCTTTTTGTCTTGTTAGCTCAAATCTAATTTGTAAAAGCAAACTTAA
TATTAAGTTGTACCAAGTATTTGTCAAAATTTACTTTAATAGGTTTTGAGTTTTTTTAAA
AATAAATATTTAATGTAAATATTTTATTATAAAATCATTTAAGTTATAGTTGGTCCTGCT
AACTTTTTTTTGCTAGGTCTGTCTCTGCTGCTTGATACCTACTCTGACACTCATGTGCTC
CAAGATTCAAATTACCTAAATTTTTTTGAAATAAAAAAGCCACATCCAGTTAACTAGCAT
ATTACTATCTAATGCTAATGTTCTCGTGTGTGTATATATATATATTGAGAACAAGTAGTT
CCCCAGACGAACAGTAAAACAAACCTAAAGCTAACATAAGGTCTACAATCAATACTTATG
TCCAAATATTCTCAACAAATTAGATATAACTTAAATTCGATTATATATGTAGAGTTTTTT
TCAAAACTTACATAATAACATATGTATTGTGTCGTGTGTTATATTTTAATAGAGTAGAAT
TGACTAAGAATAACTTCTCGTGCAATCCATTAACAAAAAAGTACTTCATCTGTTATATTC
AACATTAACAAGGTAGTTAAAAAGAAAAAAATAATAATTAATACACAATGCAACGAACAA
GTTTTGTTCCACCGTTGGAATTTCACGTGCCTTGGAAATGGACACATGCTCTGCATTCCT
AACGTAGGGAGCTTAATTATGCAATAAAATATATATAATCTGTGTAACAAACATGCAGCA
AAGAAATTCAAAATAATTATCTTTCATTTATTGATTTATCTAATATAAAAATAATTACCA
TAGAGTCAGGGGCGGACCCAGGATTGGTGGAAAGGAGAGCCAAATATGAAAAATAAAAGA
AAAAATTATACCTATCAATTGAAAGATTTTTTTCCCTCAATTTTAGCAATTTTTATTTTT
AAGTTGTTTATAAAATAATTATATATTCAAAAAAATTATTTACATCAATAATTTACTAAA
ACATTTTTTTTACATGTTTGGAAATCATCTATAATTTTTTTAGTAAAAAAAAATCATCTA
TGGCTTACACAGATTTTTTCAACAATTTTCTTTCAAAAAAATATTTTTCTAAACAAAAAA
AAATATATAACACTATTTTAACACATTATAAATAAATTTAATTAAATAAAAGTATACAAA
TTGCTATAACTAAATTTAAATTTATTTTTGAAGATTTACCGGTTAGCCTATCTAGACACG
TGCAATCGAAGGTCAAAAGAGCAGTGTGTCTCACAAACATAATGCCTAGAGGACAAAAAT
AAGAAGAAGAAGAAATAAAGAAAATATATAGAGTTATCATAAAAAAATAGAATATTTGTT
GTTATGTTTGAAAATCATGTATCTCTTATATTTGTGTTAATAAGCTTGCTCTACAATTTC
AATAACAAAGGTTTATATATAAGTAACTTTTATTTCTCAAATTAATTATAATCAAAAGGC
TAATAAAGCTATTTGATTGTAATTATAATCATTTATAGTATTTAAAACACCAATAAAAAA
TTATAAAAAGAAAATTACGAATAAAAATGAATGATACTCAATTTAAAAAGAGAAAAAAAA
TAACATTAGTTCAACAATGAAGACTATAAAAAGAAAACTATAATATTTATCTAAAAATTG
AAAATGGTCATTATCTTCATCATAAAAAGAAAATTACAATAATAATTATTACAATAATAA
TTATTTAAAAAGAAGATAGATTCACATACTCCAAGAATAATTCTTTTATAGTTATTTCTT
GTCTTTATTGTTTATTTTATTGAAATCTAATGGTTAAGATTAGTTATTTATTACAACTAT
TAAATTTAAAAAAAAATGAAAGATGAGGATAAAAAGTAACTTTAAAAAAGTTACTCTTTG
AATAAGTTGATTCCTCCTCATTTAAAAAATATGACATTATTTTTATAATGAAAACTATAA
AAAAATATATCCCAATATATATATATATATATATATATATATATATATATATATATATAT
ATATATATATATATATATATATATATATATATATATATATATATATATATATATAAAAAT
TTACCCCTATACAAAAAATTAAAATTTAGAAAAACTAAGGTGGTACAAAATAAAAATGGA
AAAATATAAATTAAATGTCTAATTTTAAGTTATAAGAAAATCATAGTTTACTCTCGTAGG
GTCAATAATGCATTCACTATCATACTCTTTTTTGGACATTTTTATCACCAACTAAAATTG
ATTAAAAATAATAAAAATCATAAATAATATTTATAAATATCATACATCTAGTTTCTCATT
AATGAGAGTTGGATCTAATAAAAGGGTTAACCTCCCGCAATTTAACTAATCAAGGTTTGG
ATCCTCATATTTTTTCAATTTCTTTCTTAAGTTCCCATTACATTAATTACAATATTATTT
ATCACATTTAATGTTTTCTTTTTTTTTTTTTCTGTCTCATTCTTTCTTCTACTCATACGC
CCCATAAGTGGAATGTAGATTAATTTTTTCCGTACTAATTCGTAGAGTAAGACATATGAC
CAACCCAATTTGTTTTATTTTTAATGAAATTAATTTAATAGTAATAAAGAATGTTTAGGG
GCTACAACCGGACATGGTTATAATAATTATTAATTAATAAATTTATAGCATTTCTCTTGG
TTCTAAAGTGCAACCTTTGGTACCAAACAATCTCTTATCTTTTCTTATATATATAAGAGG
CTTAGGCCGGCACCACACAATACTGCAAAGAGCAATCACAACCTTCACTTCCCCACAAAC
AAGAAGCA
GmGBP1 promoter2
>prG. max Wm82.a2.v1|Glyma.08G246000.1
SEQ ID NO: 117
TGTAATTTCATGGATCTTAGATGAAACTCATTTTTATTTCTTATATTTCTAAAAAAAAAT
TAATTTGGTCCAAGTAATACATCATTTGCATTAATTTACATTATTCTTATATCGATTCAA
TAAAATGATTAATTTAGATTATTTTTTATTGGTCTTTGAAGGTAAAAATATTATTTCATG
TTATCCGAATGTTAAAGAATATTTTTATATAACTACTTTTATTAATAAATTAATTATATT
TAATTTATCACTAATTTAGTATTGAACATATTGGATTACATACAAAAGAATGTTTTAATC
TTTACATAAGAAAATAATTTATTTGATAATTACTTTGAAGAACTTAATTAAATCAAATAA
ATATTGTAGTAGAATTAATATTGCTATATTATTTTTTTTCCGTAGGAATTGAAAATGATA
TTAAGACTTTACAACAAGCACACACACCATACTCAATCAATTAAGCTAGATCATTTGATA
TATTGTTACATTCTTTGTCAGCACCAAAAATATATAATTAATATTAACATAAAGAGTGTT
TTTTCTTAAATGCAGAAGTTATCCATAAAAAATAAGAATCATAATCTATTATTACATATT
AGGATTAAGTCATGTGATTAGCTACCATAGTTAATCATATTATTTTTTGGAAAGGCCTTA
AATTATGAAGGTTCTTAAAATAAAGAGAGATGAAATTATTTTAAGTCAAATATTTCTCCT
AAAAAAAGTCTAAATCCATATCTCTTTAATACCATAAATAGAAGTATTTGACTAAGGATT
AGTTTGATTTATAAAAAATATGGTAATAGACAAGATAAATATTTTTGGTCTAAGATTGAT
TTGGAAAAAAAGTTAGGGAACAATACAAATCAAGTAGATGTGCACGAGACAAAATCTCTA
ATTTTTGTTACTAAGAAAATCATCAATCAATTTTTTGTCTTAGGTACAAATTATTCAAAA
GATACATTTATGCTTATTTTTATCTATTTAATAACTAAAAAATAATTACTGACTTATATA
ATCATTTTTAAAAGAAAACACTTTTAAATCTCTCTCACTTTTATTTCAATAAACACATTA
AATTTGTTAAAATATAGTACTAATAAGGATAATAAGAACAATATAGAATAAAAATACAAA
TTACATGACAATTTTATCTAGTACATCGGACACAAGTTAGAAAAGTTTAATAAGAGTCTA
ATATCTAAACTTGAAAAGTAGAAAGATAATTTGTTATTCTTAGAATTTCACTTATTAAAG
AGATGATAACAAGATATTTATCTTAATGTGAATGCATGTAGAATCATTTTTTTTATTAAT
TCAAGAACACATATTCAAATGCATAAAATCTTTTATTTATTTATTATTGTTGTAATATAT
TATAATATAAAATAAATCTTATTATTTTGGTGCAAGAATTAGAAACACTCCATTATAACG
ATCACATTTATTCTAATTAATTGGGTCAATAATGTCTATATATCACATTCTCTCTTATAT
CTTATTTTATCATTGACCAAAAATCTTAGAAAATAAAAATTATAAATAAATAAAAAGTTT
CTATTATCATAGGTCCGGATTTTCTTTCATCTAATTAATTTTCACTAATTTTTTAAAAAT
ATTTTTCTATTTTTTTATTATATCATTCTCCTATGCATTTCCATAACAATTAATTAACCC
AATTAGATGTATTTTTCATGAATTTCTTTTGATAACAAAAAGACTGTATAGGAGCATGAT
TATTAATTTCAAATTATAGCATTTCTCTCGATCCTAAAGTCAATCTTCATGTTCTAAACA
ATCTCTTATTTTTTCTTATTTTTAATTTTAATATCTCTTTCTTTCACCCTTATAACCAAA
AATGGGGTCTAATAAACGTTTTCATAATAATTCATTGAATAAGAAATATGACCAACACGA
TTTATTTTATTTTAATGAATTTCTTCTAATAATAAAGACTCAATAGGACCATGATTATAC
ATTTCTAATGCTAACATTTCTCTTGGTCCTAAAGTTAACCTTCATTGTTTTTCCTGAGTT
TGTCATCCTTCATGTTCTAAACAATCTCTTATACACATGGAAGAGTATTTTATTATGAGA
GGTGAGAGGATTAAATAGTCATCCTGAGAACAAAATAGAAGGTCCTGATTTAATTATTAA
TCAAAATTACACGTGCATTTAATATTGGATCTATCATGTGCAATGTTTTTCTCAGTCTTG
GATTTACAATATAGTTTTAATTAATAATTAAATTTGGACCTTCTATTTTGATCAGGGTTG
TTATTTAATCCTCTCACCTCTCATAATAAAATACTCCTCACCAGATATGCCCTCTATATA
TATATATATATATATATATATATATATATATATATATATATATATATATATATATATATA
TCATTTTTTGTTTTTTAATCATTCTCTTACACCTATATCCCCCAAAATCAACATTTCCTT
GAATAAGAGATAAAACCAACATAATTTGTTTTATTTGTATTGAATTTCTTCCTTTAATAA
TAAAGATCATGATTATAAGTTTCTAATTCTAACATTTCTCTTGATCCTAAATTCAAAATT
CATATTTTAAACAATTTCTTATCTTTTAAATATTTTTTTTCATCATATCATACCTATTAA
ATTCTTTTTCTTCTTTAATTTTTTTACCTTTATTTCTCTTTCTTTCACGGTTTCGCCTAT
ATACCCAAAAAAATGGAGTGTATTAAACATTTTCATAATAATCCATTGCATAAAAAATAA
GACCAACACAATTTATTTTATTTTTAATCTTAACAATAAAGATAGTATAAGACTATGATT
CAACATTTCTAATTATAACATTTTCTCTAGTCCTAAAGTCAAAATTCTTATTCTAAACAA
TCTCTTTTTTTTTATTTTTATATATATATATATATATATATATATAATAAGAGGCCTAGG
TCAGCAGCACCACACAACAACACTGCAAGAAACTACTTCTCCCAAACTCAATCCACAGTC
GmGBP1 promoter3
>prG. max Wm82.a2.v1|Glyma.18G266900.1
SEQ ID NO: 118
AACTGGTTGATGCTGATTATAAAAATATTTTTAAAAAAAAATTATTTACTGTGGGCCATT
GCCAACGCTAAATGTTTTGCTTTACTGCTAATTTTTGAACTAGTGTTGACAATGAAAAAA
AAATATTTACCGTGGACAATAACTAAGCTAATTCTTTTTTCCGCTGAAATTAAGAACTAA
GATCGAGATAAAACTCAAATAAAGTTGCTCACACTTTACCTTCTTATACATTTTTATTTT
AATTTATTTTCATCATTGCTGGATTGAAGAGGTCCTTTTCAAATGGTCTCGGTGTGAATA
CTTTATTGCTTGAATAACAGGCATTCAGAAATACTACAAGGGTTAGAAACACTCCCTTAT
AACTATCACATTAATTCTAATTAATTGGGTTGTCAATAATGTATTTTCTTTTTATCACAT
TCTCTCTTATATGTTATTTTATCAACCGCTGATTTTGGTTTTGGAATTTACAATGATCAC
CATTACCATTGGGGAACTTCCTCTATGGAATTGCGGTTCTTGCAAAGATTGACCCTCCAT
GGGGACAAAAGTTTATTCACTTGCGACAGATTTCATGAACTTGGGCCAAAAGTATAACAG
ATTTTATCCACGTCTAAGGTGTGTTGACCGAGTTTGAGGATGGAAGGAATCAAGAAAGTA
CAAGTGAAGCTGTGAATGCATACTATTCAGCAGCATTGGTGGGTCTTGCATATGGTGACT
CAAGTCTTGTTGACTCTGGGTCAACGCTAGTGGCATTGGAAATTCTAGCCGCACAAACTT
GGTGGCATGTGAAATACAAGAGGGATAGTAAGCTATGGTGGGCCAGTGCTGAGTGTAGAG
AGTGTAGGCTTGGAATTCAAGTGCTGATTATGCCAAGGAACTTGTGGAATGGACATGGCC
TTCTGCACGTAGAGAAGGGTGGAAGGGAATGACCTATCCTTGCAAGGAATTTATTATAAG
GAAACAGCATTGGAAAATATAATTTTGTTGATGGGAATTCTTTCACTAATCTCTTGTGGT
GGATTCATAGTAGATGAGGACTGATGGATGCACATAGTTTTGTGACAAGTCCAGGAAATA
TCATGTTTCAATGTTATATTTAATTTGCAGTAATTTATGAAACTTTGTATCTTCTCATCA
GGTTATTGTTATTATGGTGGTTTAAACTTTGACCACTGCATAAAGATCTTTAGAGTTTAT
GCAAATGTAACGCAATCACACATGTAAAATAAAGATTACTCCACTTCATAAAAAATAAAC
TCACAAGAATAATGTTTGATTTCTGCTATCCACGTAAGAATTTTATCGGTAAATAATTAA
AATATCTTTATAAGCACTCTTTAATTTTTGAGATATACTGATAGATACTGATGTATTTCG
AAATCTAAATTATCTAAACTTTTTAAAATAAACAAAAAATTACATCCAACTAATGTATTA
CTATCAAATGCTATTAGTGTTCTCATGTATGTATGGGATAAGAAACTCGCATATAATACA
TATTGAGAAAAGGTGCTAAAATGATCGATCAAACTCGTTAGTTTAATTAAATAAATGAGT
CAAATTTTAATCATTCATTTTTTCACAAGTTAAACTTAATTTTCTAGTACTCAATTTGGT
TCATTTAGAACCCTAACTAACATAATGTCTACAATCATACTTATGTGCATGTATTCTCAA
AAATTAGATCTAACTTAAATTCGATTATACATGTAGTATTTTTTCAAAACTTACATAATA
ACATGTGTGCCATATTGTGTCGTGTGTTATATTTTAATAGAGTAGAATTCGATAAGAATA
ATTTCTCGCGCAATCCATTAACAAAAGTACTTCAAACCTGTTATATTAAACATTAACAAG
GTATTTAAAAAGAAAAATAAATAAATTTATACACAATGCAACGAAAAAGTTTTGTTCCAC
CGTTGGAATTTCACGTGCCTTGGAACTGGGCACATGGTCTGCCTCCTAAACTTAGGAAAC
TTATGCAATAAAATATAGTCAATCATCGACACCCTCCAAGTTAATAAAGATGCAACAAAT
AAATCCAAAAACAATAAATGAAAGATAATACTATATATTTTCATTTTTTTATCGATCATT
TAAAAAATATTTCTAATTATTCATTATTTGAAAGTTTCTAAAATAACATTAATTATATTT
TTTCAATTATATCCTTGTTAAAAAAGAAATAAATAAATGATATTAAAGTAGTAAATAGAG
AGAGACAAATGACATATTAAAAATTGTAATAATTTTGATATAAACAAAACTATCAATTAT
TTTTTTTAATCTATGTGTTGTAATCTTAAATGATAATATTAAAAAGGAAAATTTATTATA
TTAAAAATACATTAAGTACTTAATATTTTTTTCAGTAATGTAGTACATTTAATTAAGAAA
ATATACTTGATGCTCCGAATCTATTTAGTTGATGTATAACAGAGAAAACTTAGTTTTTAA
TATTATTATTATATCTTTTAAAGGAAGGAAGAATTACCATATATAGAGATGTAAGACTAG
TGTAAGGAAAAATATTAAAAGCAAAGTGTTCTTTTGTTATTATTTATCTAGGAAAAATAA
TTATTTAACATTATTTAGGTTGTATACACATATAAGAGAGAAATAGAAAGAAAAAATATA
TGCAACTTGTGATTTAATGAAGAAAAAAAATGAAGACAAAAATGAGCTATATAAGGTTGT
TGACCAACACATGTAATGCAATATACAATATGCAAGAAGCGTTATTCTAAGAAGAATGAA
AGTGAAACACCCTTCAGATCCGTGCAAGATACAATATGCAACAAGCATTATTTTAAGAAA
AATTAAATATTCCATTGTATTTTTATACCTTGGAAGAGAAATGAAATGAAATTATATGCA
TTCTCATTATAAGAATGGGTCATTCAACACTATTTAAATAGATAATACGTTTATATATAG
ATCCCTGCTGCGTTGTTTCATTCTATTTTAAGATTAAGAATAGACATAATCAAACATATT
TCTTAACATATCTCTTATTATATATTTAGAACATTTTGGAACAAGAGGATCATTTATAAT
GAAAAGGATGAGCTTCTAAGTTCATCCCAGAGTTTCAAGAAAATAATGCAGAGCTTGAAG
AAAAAAAGTCAATCTCTCTCGGTGTAACTAACAAGATTTCGATCTTTTCACGTATCTTTT
CTATTTTTTTCTTCGATCTCTATCACATTAATTGATTTCTTTTTTTATCTTTGTCTCCAT
CTCATTGTTTCTTCTACTCATAGGCTCTCCATAAGTGGAATGTAGATTAAACTTTTCCGT
ACTAATTCGTAGATCGAGTAAGACATATGACCAAACCAATTAGTTTTTATTTTTAATGAA
ATTAATTTAACAGTAATAAAGCATATTTAGGGGCTGCAGAAGGGTATAGTTATAATTAAT
TAATGCATGGCATTTCTCTTGTTTCTAAAAGTCAACCTTTGGAACGTACCAAGCAATCTA
TTATCTTCACTTATATAAGAGGCTTAGGCCGGCACCACACAACACTGCAACGAGCAATCA
CAAAGCAACG
GmGBP1 promoter4
>prG. max Wm82.a4.v1|Glyma.08G245700.1
SEQ ID NO: 119
ATAACAGGCCGACAAAATGGCTTCCCCCCACATTTGGGTAGACATACCAGAACTTAATAA
CATAGCGTTCATCATTTCTAGTTATTACTACTTTGTCAGCTTCAAAAACCAATTTTAATC
CAGCTCTATTTAGACAACTACCAGAAATTAGGTTCCTACGCAGAGAGGGAACATACAAAA
CATCACTCAAAGATAATGTTTTTCCAGAAGTAAGTTTAAGGAGAATCGTTCCTTTACCAA
GCACTCCAGCTGTAGCTGAATTTCCCATGAAAACACATTCTCCGTCATCAGCATCCTCAA
TTTGGTGAAAAAGCTCTTTGTTGGCGCATAGGTGTTTAGAAGCACCGGTATCTAGAATCC
AGTCCACCTTGTTGTCGACCATGTTTGCTTCCACTACCACAGCAGCTATCACTTCTTCAT
TTTCTGCTAGATGTGCTTGAGGAGCATGTGGTGCAGCAAGTTTCGAGTTGTTAGATTGTT
GTCCCTTCCTCAGTTTTCACTCATAGGCTTTATGACCTGTTTTGCCACAAACATAGCACT
CACCCTTTCTTTTCTGAATCTTGTTATCACCTTTGTTGATATTAGTGTGTGTTTTCTTTT
GTCCTTTTTTCTGAAATCATTTTTCTTTTCCTTTTGACCTGTATTGATCAGCAAACTCAA
CAACATTAGCATTAACTGAATTTACAGAATGTAAAACATGCTTATTTTAAGTCGGTTGGC
CTCCTCTGTCTTCATATGATTTATTAATTCTTGAAGTGACAAGTCCCTCTTCTTGTGTTT
CTGTTGGTTGTGGTAGTCACTCCACGAAGGTGGAAATTTTTCTAGAAGCACATTAGCCTG
CAATATCTCACACATCTTCATTCCTTCATTTAGTATATCACCAACTAGATTCTCATACTC
ATGAATCTGCTCCATGATTGGTTTGTCATCCATCATTTGGAAACGCAGCCAATTTCCTAC
CACATATTTCTTTCTTCCAGCATCGTCATCGCCGTAACGTTTCAGCAGAGTGTCCCATAT
TGTTTTCGCAGACTTTTGGTTAATGAAAAGATCAAACAGATTATCTGCCATATGAGTTAG
CAGATGACCTCTAGCAGTTTTGTTGTCCTTTTCATATTTCTTCTTTGCTTCTTCGTCAGC
TTTCTTGGTTTCTGCAGGAAGCGGTGTGACGACAGGGGTAGCAGGAGTTTCAGGAGTAGA
TGATTCAGAAGCGTTGACATTAGCGGGAGGATCTTCAAATAGCACATAATCAACCTCTAA
GGCTTCAAACAAAATTAACAGTTTTTTAGACCATCTCCTATAATTTGAGCCGTCTAGAGG
TTCTATTTTCGACATATCCGGAATGATTTTTGATAGATTTCCAGCCATTTCTGTTTAGTA
CTTGATGCAATGAAAAAATAATTTTGTTTTGCTTTCAAATTTGTTGGAAAAACTAGAAGT
CATAAGTACAAAACAGAAACATATAACACAGATTGGAGGTTTGTTGTGCCGTGAAAAATC
ACTGCCTTGAAAGCAAAATTCGGCTAGACCTTGATGCAATCTAGTTTGCCGACTGCTCCC
CAAGGTTAACACAACGAGCCTATGAACACGTGCACTCGTGGCCTTAGGCTATGGAAATCA
CAGAACAATTGCCCAGAATTTTAGAAGTAGATAATAAGAAAAGAATTGGAAATTATTTTC
TGCCTGTGCAAGCCTGTGCAAGACTATGACAGTCCTCTTTTTAATAGCAAAACCACAAAA
ACATCTTTTTCTTTTCTTGTCCACAAATCATAATTATTAGCTACACATTTAACAAATTGA
CCGTTAGAGAATTCCTAATAAAGTGGAGGTTTTGCTATTTGATTTATAGCTAATCAAGAG
CATTAAAACAAAGGAAATATTTGTTAAAAGAATAACAATCAAAACTGTTTAAAATCAGTT
AGGAAATTTGGGAATAAATTGTGGCTTCAGTTTGCCATTGTCCAACAGGGCTAAGAGTGA
AAACGAAGATATTGAATTAGTGTGGATTTGGATAAGTGTTATAAAATTAATTTTATTTAA
AATTGATTTTGAAGTGATGTAATTTATATTTAAATATTTTTATTATAATAAAAATAATTT
AACTATAAAATTAATATATATATATATATAATTATTTTAATTCAAAATTAATTTTAAAAT
CAATTAATTTTGATTTTTTAAAGTAAAACTAACTATGTAAAAGACTATTTAAAATATATA
TTTTTAGCATAAAATCAAATACACATTTTATAAATTCAAATTAACTCATTTAGCCTACAC
TATAAAACAAAAGTCTCTATATCAATATGAACAAGAAAGAGATTATTGGATAAGAGAAAT
ATTTGTAACATTTCATTCTTCTGACTATCTTTCTTATCCATTTTTAGTTTATTAAAATTT
ATTAAAAATTATAAATTTTAATGAGTCTCATATATCATTTAATGATATTTTCTCTTGATT
TAAATATAAAACTCATTAAAATTTATTATTTTCAATAAATTTTAATCAATCATAAAAATC
ATCTCTAACATTCCCCCGCTGAATATGCTTTCTATAGTAACACAAGCCCTAAGCTAATAT
GGTAAATCAGAGGGAGTATTCACATACTAGAAATAAATTTAGTCTCTTAAACATTAAGAG
AAGTAAAAAAAAGAGTGTGTGAAAAAAAAATCTCAATAAATTAGTTAAGATATATTTATA
TTTAAATCTAAAATAATGATTCATAAACATCCTTGGACTTCAAACACTACATTAATATCT
TTCATTTTTCTCTTCTCTTTTTATCTATTCATAAACATTATCATTTATATTTTTCTCTTC
TTTTTTTTCAAGTGTTAAATGGCATATTGAAGTGTCCATCCAACGTTTCCTTACATTAAA
AATCGGTGAACCCGGAAGTAGAATTTGTCTTGGTTCTAAAGTCAACTTTCATGCCGAACA
ATTTCATATCTTTGCTTATATAAGAGGCTTAGGCCGGCACCGCACATCAACATTGAAAGT
GCATTCAAAACTCCAATCACAAACAAACT
GmGBP1 promoter5
>prG. max Wm82.a2.v1|Glyma.18G267100.1
SEQ ID NO: 120
AAATTTTAAAAATGTTAATGTTGTCATCATAAATTTTTTTTATTAATTTAATTTTTGTAT
CAGTAAAACATATTTTTGTTAGTTCTATAATCTCGTTATAATAAATTTAATTACTTTCTC
TATTTATATACTCAAGCACAATTATTATACTAAAATCTCCTTGTTTCATTTTTCCCAACT
CTCATCACTACATAGTATGAGAAAATTAGTGAAAAAGTGATACTAAATTATAATTTTTTG
GGATGTTTACATGTTTGTTAAATTTATTTTGGAGGATTTATCTATCACAAATTGTTCATT
CATTTGTAATCAAATTACATGTGTAACATTAAATTACCATTTAATAGAGTTATCATTCAC
TATAAGTAAAAATATGTTTTTTTTCATCGGGACCAAATAAATATAAAAACTTTTATGAGT
ATAAAATTAATATTTTAAAATTTTAAAAACAACCCAAACATATTTTACACTAATATTTAA
TGCAAACTTTTATCATGTATATTTTTTTTACTGCAACTTTTATCATGTATAAAGAGTTTT
TAAAATATCATTTCATCACACATTGTCATGTACTTTTATTCATCGCGGTAGAAATTGGAT
TCAAGCTTTATTTCATTGCACAGATTTATATATATGAACAAGATTAATTGAGAACATAAA
AATTTATTGCAGTGATGATATATAAAAAAATAAACTCTTATTTATTTCATTCTACTTCTT
TTTTTCCCCATCAATTTGAACACAACCTTATTGCTTGTCTTAATTTTGAATCGAGATTCT
CCCAACAAGCTATTTTTCTTATCACAAAACGAGTTACATGGAAGTAAATGCCTTAAGTTA
TATTTTTAAAAAATAATATAAAGTATATATTTCAACTTTTTATTCATCAAAATTGTCCCT
AATTTCATAATTATCATACTTTTCATCAATTAATAGTTTTATTACTACAAAAATGCTCAT
AAGTTTCTTAATTTATAATATAAATTTGTTTCATCTGTTGGGGATGATTCCGAGTTTTCT
AATTATTAGTACTAAATTAAGTGCATTCATCAATACATCATTTTTTTATTAGAAGCAAAT
GGGAGATTTTATAAGATTAATTATTTTAATTAAATTTTGTAGGAGCTAAGAGCTCTAAAA
AAGTCTTATTTTTGAAATTGCATAAAGATTATGCATGAAAAAGGTATAATAAATAGTTTT
AGATGGATTTACATTGCTTCTTTCCTTTGGGGGAAATTTTGCAAGAACATGTACCAATTC
ATCCCCTTTCTAAGGATCACTCCCTCGGTTTTTTTATAAGTGTCGTTTAAGTTTTTTATC
CGATCACCAAAGAAATTTATTACTCTGCCTGCACCGTTTTGATTTAGTGTAATTGTGAGT
ACTTTTTCTATCATGCCCTTCTCTTTACTTCTCCCACTAATGCACGTTTTTGGAAGGTGG
TACTAGTGCTTTTCTAATCAATTGCTCTCTCTCCATTACTTGTTTAATACTTGTGGGTAT
CATTAATTGACAATTGGTTCACAACATATATTATTATAAAAAAATTGGTTGGAAATATTA
ATGTTTTTTAGAAGATGAGAGGATAAAAACTTTTGTCAAGTAACTATCCACTAAAGTTTT
GACCAATAGGTGATTAATTTACCTTTTAAAATTTCAAATTACTTATTATTTAATTTATAA
ATAATATGTGAAAACAATTAATTAAAAAAAAGATAAAATAGACAAAAAATTAATGTTCTC
TTGAATATTTTGTTAAAATATGACACTTACAAGAGAACGGAAGGAATACTTTTTAACGGG
TTCAAATTATAGATATATGCATGAATTAAACAAAAAATATAAATAAAAATTAAAAAAATG
AAAGAATTTGTAGTTAAATTGAATAGTACACCATTTTCCCAATGCATCTTATGGGTTTCA
ACGCACACCTTTTGGTCTTTGGTCAAGGATTTCAGCTTTTAATTTCACAATTATTACAGT
CGATACATATGTGAATTTATGTATTGTAGCTGAAGTGCTAATGGGGGTAATCACAATCAA
TGTGATTTTTATTGTGTTTTCAGCATTACCATTGTGTTACACGATCACCACTTGCAAGGC
AAGCACTACCGTAGCTTTCAAGTTTCAACATCCAAATAGACAAAGAAAATAAAACTGAAC
GGAAAAGCCCGCATAGTATAAAGTCCACAATCCAAAAAGAGACTTGTTTACAACGGCAAT
ATTCTCAACAAATATGTACTATAATAACAAAATTACCATATACGCCAACATTACGCTGTG
TTATATTTTGGTAGAACAGAGTTAGGCAAGAATGAGAAATTTTAAATCCTCTTCCAACTT
CTTCCTACAACTTTTCTTACTTAATTTGTATAAGGTATTGAGAAGATTTATAAAAGAATA
TAGTACTAACATTGTTCCATAAAAGCTCTTCAAACATTGAAAACTGAACACAAAGAGACA
GAGGGGTTTAACAAAGCAACGAAAATGTTTCCGTTCCGCTTGGAATCTTTACCTGCCTTG
GAAATGGTCACATGCTGTGCCTCCCTGGCATGAAAAAGATTTGGAAATAAAACACTAATA
ATCAATCATCGGTATCCTCCAAGTAAACTGCATGCCAGCTTATACAGCAATATTACAGTA
AATTATAAGTTAAGTGGAAAATATAAACATTTACCACTCAACACTAATTGCATAGCTTTC
AATGAACACTATCTTGTACTATATTCCTCTTCACTCTAATAATAAGTGGTGAGCATAATT
ATTTTAACTACAAACATTTACCTACTCCACTTAATTGCATACCTTTCCTTTAACACTATC
TTGTATTTTCCTCTTCACTCTAAATGTTTTTTTTTTACCATATCTTAACCAAACACAAGG
ATTCTGAGGCAAGATATAATAATACACAGAAGCATTATTCAAAAGTCAACTTGCAAGTCT
AGAAAAGCTCTCAGCTTAGCTCATATAAGAAGAGATCACAAACATTGCAATAAGCAAACC
ACAAAACTCCCAACCCTTTTCTCTTCCCCCACACAAACA
GmGBP1 promoter6
>prG. max Wm82.a2.v1|Glyma.08G246300.1
SEQ ID NO: 121
TATACATCAACTAATTACATTTTATTAGTTCAACGGTCATCTATGATGAAGGTGCATCTC
CACTGACTAAACAACTCAACAAAAGGTGCATCTATATGAAGATGAATCAAAATAGTTATT
CATAGATAAAAATAATAAATAATAAATAAATTATATTTTCATTAATTTAAAATTAACATA
TTTATCTTAATTTTTTTTAAAAAAATATCTCATCCAACTCTTTAAAAATTGAAATACGTG
ATTTGATTTTAATGAGAGAAGTTTAGTTTGTTTTCCCTCCTTATTTCTTGTTTTATAAGT
ATACTTGAAAAAAATTATCCAATCTGAACCGTAGTCAAAAGAAATCTTTTTTTCTAAATG
ATTAAGATTATGAAACCTCTTTAAGTACTTGATCAATATTTTATTCAATTTTATGTACGT
TTTCTATGTGGCATATATCTTGGGATAAGAGTTTAATTTTAATTTATCATATCCAGTTTT
GTCTTTTAATTAAAAACATATTTTATAAGACTTTAAATTTTTTCTTTCATAATAATTTTA
CTCATCTTAAATACGATTTTCTTCCTTGATACTATAAAAAAACTTTAAAAGCTTTAAAAG
CATATTTTGACATACCATTTAAGTAATCTAAATTTATATAAATCGAACTAAACTTTGAAT
AAGTCAGCAAATATATAGTTTCAAAATAGTCTCATAAAATTTCAATAAAATGGTAATAGT
AGCACTAATAAAGCTATTATTATGTTATTTATATTTATTAAAAATGGGCTGCTTGACCTT
CTCCTATCTTCTATTACAATTTAAATAATTAAAATAATGAGAAAACATGTATTACGATCA
TATAAAATTTTACATTGATCAGAAAAAAAGCTATCTTATACATAATAAAATTTAGCTCAC
ATTGAATAACTAGAATATAATAAAGAAAAAATTTTAGATGTAATGTTTTAACTGACATCT
GAATAAGTACAATATAATAAAGGATAAATTTCAAATGTAATTTTTTAACTCACATCAAAG
TTGTCCCAAATAATTTTAGAATTTAATGTTTATGCATTGATGTGTAAAATAGTTTTATAC
CATCATTCGATCTCAAATTATCGTTTAAATTATTTAAAAATAATTACTTTAAAAGTGAAT
AAACTTATCATATAAAATGAATTGTGATTGAATAACTATATAAAAAATTTTACATTATAC
GTGCATAATTCTTTTTCTCATGGTTTTATTACTACAAAAATATAAGCTTCTTACTTATAA
TATAAAGCTGTTTCATTAGCTGGGGGTGATTACTGGAATTTCTAATATCAATTACTAAAT
TAAGTGCATTTATTAATACATGCTTTTTTTATTAGATGCAAATGGGAGATAATTATAAAA
TTAATTATTTTAATTCAATTTTGTCGGAGCTAAGAGCTCTAAAAAAAGTCTAATTTTAAA
AATTGCATTAAAAGGTATAATAAATAGTTTTAGATGGATATTCATTTGTTCTCTCCTTTG
GGGGAAATTTATGCACATCTTGTGCTGAAATTAAAACCGCTTACAAAAAAAAATGTACCA
ATTCATCCGCTTTTTAACGGGTTCAAATTATATATCTCCCACGTATGTGCAGGAACTAAA
AAAATATATAAATAAAAACTAAAAATGGAAGAATTTGTAGTTGAACTGAATAGTACTACA
CCATTTTCCCGATGCATCTTACCTGTTTTCAAGTGGGAAAGACATACGCATTTTTTTTTT
AATGAAAGTATTATTCAATGTCAAAAATTAATATTGAAATTTATTAAAGAAGTTAACTTC
CTCCAATAAATATTTTGAATGTGTTGGTGAAAGAGATATTTAGTTGCAACTTGCAAAATT
GGAGAAATCATTCAGTGTTGTAAAATTCAACGCACATCTGGGTCACTGGTCCAGGATTTC
AGCTTTTAATTCCACAATTATTAGAATCAACATATGTGAATTTATGTGTTGGAGCTGAAG
TGCTAATGAGGTAATAATAATAAATGGTCAGGAACACAATCAATGAGATTTTATTGTGTT
TTCACCATTACCATTGTGCTACACGATCACCACTTGCAAGGCAAGCACTACCGCAGCTTT
CAACATCCAAATAGACAAAGAAAATAAAAAGTGAAAACTGCACGGAAAAGCTGGCATAGT
AACAAAATAACCATATAGGCCAATATTATGTCGTGTTATATTTTGGTAGAACAGAGTTAG
ATAAGAATGAGAACTTTTAAATCCTCTTACAACTTCTCCCTACAATTTTTCTTACTTAAT
TTGTATAGTATAAGTAACTAAATATTAAATATATTTATTTTTTTTATAAAGTATTGAGAA
GATTTGTAAAAGAATATACTAGTAACATTGTTCAATAAGAATATCCTGTGCAATCAATTA
ACAAAAGCTCTTCAAACATTGTAAATTGAACAGAGAGAGACAGAGGGGTTTAACAAATCA
AGGAAAAAGTTTCTGTTCCGCTTAGAATTTTTACCTGCCTTGGGTGTTCACATGATCTGC
CTCTCTGGCATAAAAAAATTATGGAAATAAAATACTACTATAATCCATCATCGGTACCAT
CCAAGTAAACTGCATGCCAGCTTATACAAAGCAACAAATGAAAAACAACACACAATTACA
CACTAAATTATACAGTAAGTAATAAACATTTACACACTCAACACTAATTGCATAGCTTTC
AATTAACATTTAACACTATCTTGTCTTTTCCTTTTTCCTCTTCATTCTAACAAGTGGTGA
CTATAATTATTTTAACCACAAACATTTTACCGACTCCACTTAATTGCATAGCTTTCAATG
AACACTATCTTGTAATTTTCCTCTTCACTCTAAATGTTCTTTTACCATAACTTAACCAAA
CACAAGGATCGTGAGGGAAGATATAATAATACACCGAACCATTATTCTAAAGTCAACTTG
CTAGGTCTAGAATATCTCTTAGCTTCATATTAAGAAGAGACCACAATCATTGCAATAAGC
AAACCACAACTCCCATCCCCTTCTCTCTTCTCCACAAACA
CcGBP1 promoter1
>prC. cajan_rna-KK1_019357_Cc_Asha_v1.0
SEQ ID NO: 122
ATCCAAACCAACAAACCCAGCCACATACAAACACAAAACACAATACATAGGCTCCATACG
ACTCAAAATAAACTCAAATCTCACATGCATCCAACCTATGCATCAAAATCATCAAAATCC
TCTTTTTTCTCAAAACCCCAAAAGATGAGATTTTCTTCAATCCATCTATGGATTCTCAAC
ATTCATGCACCTATTGAGTTTCTAAGCTCACATGCAACCTACACAACAAACATTCTAGCT
TCCCTTACCTGAAGGTTGGCTCAGAATAGCCAACAACTGTGACGAAATAGAATCCCCTTC
CAAAGCTCAACCCGGAACTTCAATCATCCCAAAACAAGCTTTCTAATCTGAATTTCAAGA
AAAGCCAGAAGGCATTAGTGGTCTAGAGGAGAAAAGGAGTCAAATAGAGAAATTTGGGAG
AGAAAGAAGCTCACTGACCCAGAGACCTTCTCTCTAAAACTTCTCTCTAAAAACTCACAT
TTTCCTGAAGGCCCCTAACCCCTATTTATAGATTTTCGGACTTCCCCAGAACGGCCCGTA
CCGAGTTCGGAACGGCCCGTTCCGAACTCCTAATTCACGCACAAAAACCACGCCCAGAAC
GGCCCGTTCCAGATTGGGAACGGCCCGTTCCGGGCTACTAAATTAGCTATTTTTTTTAAC
CACGTTACAGATATTGCACCTAAGCTGGTCGAGATAGAGAGCCTCAAATAAGAATGACGC
TCGACATGATATCAGAATTCTTCACTTGGAGAAACCAATTTTAAGTAACAACCTAAAACT
CAACAAAGATTCAACACATGCAACACTATAAACCAACTATATACATGACAACCAACAAAA
AAATGTACTTCAATTCAACATGCAATAACTAATTTATTTTGCCATATTCAAACCAACATG
CTTCGAAAACCCCAATCCCAACTTAGGTCATTGTACTTAAAATATGGAACTCATTAGTAA
CTCGAATTAACATCAACCATGACAATGCAACAACTTTACTATACTAAAAATTTTCTAGCT
TCCCTTACCTCAGAATCGGAGAGAATTGGGGAAGGAGAAGAAGGAAATCTTGAACGACCC
ACAACACAACTCTAACAATGAATCTACAACTCAATTCAAGGCTCCTAAGCACCTGAATTC
ACCAACAAGACTACACAATGAGAATCAAAGAAGGAGGAGAAAAAGATTGAGAGAAAAGAG
ATGCTCATGGACTAAAGTCTTTCTCTCTAGAATCCTTTCTCTAGACTTATGAGAAAAAGA
GTTTATAAGACTTTATTTAAAACAAAAATATAATATACTTTTTCTCTTAGGATCAACCAA
CTCTTTGCTGAAATAAACTCCCAACACTGAAACTCTCAAGGGATCCTCAAACTACAAAGC
CTCTCAACATTGATTAACTGTTCAACTTATCGACATGAAACAACTTAGAATAATTATAAC
ACTTCACGACTTATAATGTTACAACCTTTAATGAAGTTTCTCATTCCAATCACTTTTTTT
TCATATACAATATTTAAATCCAAGACTTTGCTTAATAAAACCTGTTATACAATGCATAAC
ATTAAAAAACATATAAAATTTTGATTTTTTTTTTATTATAGATCACAACTATTTTTGATG
AGACAATCTTATTATGGTTACGAATTTTTTTTAAAGTATTTAATACAATTACATATTTAT
TATTAAAACTAGAACTAGAGTCCTCAAATTAAACATAGATAAACACCATTTACTTAATGC
ATCCTACCTGTTTTCAAATTAGAAAGAGATATGCAGTTGCAAAATTAGACAATCATTTGT
TTTTATTCGTATTATCTAATTCAACATATAACTTTTAGTCTGAGATTTCAGCCCACCTGA
CTTTTTTATTTTCACAATACCAATCAATATATGTGAATTTACTTATTAGAGAGATAGAAA
AAGATGATGTTCTACAATTTATTGTACATTTATCATTCCTCAAGCTGTAGTGCTACACTA
CAGAGATCATGACTTGCAAGCACTAACGTACCTTTCCCCTTCCAAATTAAATTAGAACAT
AACGGAAGCTGGCATAATTTGTCTACATATAATCCAAAAAATACTTGTGTGCAACGGCAA
TATTCTCAACAAATTGGATTCATGTTAATTTTTAGATATAATAAATTTTAATTTACCATA
ATAAATTAATAATCATTCTATATGGCAACATGATATCACGTGTCAGATAAGAATTTATCG
TGCAATCCAACAAAAGCTCTTCAAAGGATACTATTTTTAAACGTTATACAAATCACTGGA
AAAAGAAGACATCGGCAAAACAACGAAAAAGTTCCTGACATGCCTCGGAAGTCTACAATT
CACATGCTCCGCCAGACTGTCTCCTTCGTATAAGAAGTTTTGGTAATAAAGTATAGATTT
AAATACTTTTTCTTTATAATTTTATAAACTATATTTTTCAATTCTTATTAAAAATATACT
ATTTCAATTTTTATATATACTATTTTTATTTTCTTTTAGTTCTAAAATAATAAACGACAT
AGACTAACAAAATGATATTTAGAGATTTATTACCTTTTGTTTTTAACTATTAAAAAACTA
AAAGGTAATACAGTTATTATAAAAAAAACATTTAAATCTAAAATATATAAGCAATCAATC
TTCGATATCCCCGAGTTTTTAACTGCCAGCAATAACAAAGTTAATAAATGAAAAACAATG
TATAATAAACATTTAATTAGTAAACACTTGATTCAATTGATTGCACCATTTAGTATTCTC
ATTTTCACTACCAAAGGAGTATAAGAAACGTGTGAGAAGATAAATATTCCTTCGCTACCT
AACCATCAAATGCAAGGATTGTGAGGGAAGATACAATATGAGGAAGCATTATTCTAAAGT
CAACTTGCAGGGCCAAAAAATCTCTTAGTTCATTTAAGAGGGCACAAACATTGCATTAAG
CAACCACACCACAACTGCCATCCCCCTTCTCTTCTTCCCCTTTGGCAGCCGTCATAAACA
CcGBP1 promoter2
>prC. cajan_rna-KK1_019354_Cc_Asha_v1.0
SEQ ID NO: 123
TATTTCAATTATCCATTCTGTTCATCTACGCATTTATATGTATGGATCCCATTGCTTGTT
AATTATTGAAGAATCTTTAGAAACAAGTAATTACTAAAGTCTCTTACTACTCTGTGTCTC
GTTCTATATACCTAAATTGAAACATTTTCGTGCTAGTCTAAATAAAAAGAATCTACAAAT
TCCACCGTAAACAGAAAAATATTTAAAAAATTGATTCCACTTTAAATTTAAGTACACCCA
AGTCAACACATAAGTAGAGTTGAGTCATGTAATTAGATTTAGTCCACTTAAGTTAACTTC
ATCTTCAAACAGAGTCTATTGATTTCTCTAGCATAGTAAATCATCCCCTCAAATTCAAAT
CTTAAAACTTACAAGTGACAAAATATTATTCCACAATTTAAATTGAGGGCAATTTTATTG
ATAGAAAAGTATTACTTAACAAATTTGGAAAAGGATACTATGTAAAGTTAATGATGGAGA
GAGTGGTATTGATTCTATTCTAACTATTGCAAAGAAGAAGTTGTTTATAACTTCTTTTCC
TATACAAGAACTAAAAGAGAAAAATAACTTCGAACAATGTCATATGGTCAACGATGTCCG
AAATCAACAATCCAAAGAGGTTGGATTTTTTTTTTTAATGAATTACAAAGGGCAACAATT
GAATGATTAATTACCTTTTTAAGCAAAAGAACAAGAGAGATTAGCTGCCATTTTCTAAGA
GAAAAAAATTATACATGTGCTTCAATTATTTTTTATTTTTTGTGAAAGGAGTTCTCTTAG
TTGTAAATATTGACTCAAGTATCTTCAAAGGTGGTAGACATCACTTGCTTCTTTATTAGA
TTTCTCTTGAGTATTGATCAGTGTGGCAAAACCTCCAATTTGAGGATCCATGCGATCAAT
CATTAAATGGATCATAGTTTCACTTTCGATCGTTAAACGGGAGAATATGAATTGATACCA
ATCATTATAATTTCACAATTGGAGAAATAAAGATTCAAAGCTCTTTGTTACTCTATTAGA
ATAAAGAAAGAAATGTTTATTTCTAACACATTTTGAATAATACAGACGTTGATTTATATA
AACAAAACTAGTCCTAAAGTAAAATATCATGATGAATTCCTATATATTAAATATGAATAT
CAGGGTTTGAATCCTTCCTTGTGTATCTAACCGTGTTAAATTTTGGGGTCAGCTATCCTA
CCCTTTGGGATATCTCCAACGTAGCGAGGAGATTAGTTATTGTTATCGGTCGGCGGTGAA
TACTCCAGGAACGAACAAAAAAAAAACGAATAATTTTTTAAAATAAAAATATTTTTAAGT
TATTTTTCTCTAATAAAATGATTTTTAAATTTTTTTTATCAAATTCATTTTTTTATTATA
ATTATATATAAGTTAAATTTTAATATTTTTAAAACAAATTATTAATAAATAAAAATAATC
TTATACCACGATATAAATTATTTATCATGACTATAAAATATAATTTATACATCCAAATAT
AATATTTATTCGTATATATAATTTATTATAAATTTTAATTATATATAATAAATATTTAAA
TTGTGCATTATGTATAATAAAATACTAAGTTATATAAAATAATGATTAACATGTGCGCGT
ACTAAAATGCTATTTAAGGGGAAAAAAAATCAGAAAGTAATAAATAACCCTGTCACTTAT
ATATGATTTGACCATAGTTGAGATTTTGTTTAAAATTAATTTTTGAGCACATTTAGAACA
AAATCAAACTTGAAATACTCTTGATCTGTATAATTAATGTATAAGATCGAAAAAAAGTAG
TATTTTATATTATTTCTATTTATATATCTTTTGAAGGAAGGGAGAATCCTCGAGAGCAAG
ACTAGTGTAAAGTTAGTGTATATGATATTAAAAATGGGTTTTCTTTTATCATTATCTGTT
TTAAAAAAATTATTATATAACTTTGAGTGCATACAATTAAGAAAGAGACAAACAAGAAAA
AAATGTGATTTGTGATTTAATTAATAAAAAAAGGAGAGAAAAAGTTACAAAATTATTAAC
TAAATATGCAAAATGTGATGCAAGATACAATATGCAAGGAGCATTATTCTAACAAATATG
AATCACCCTTCGAAAGTATGCAATTATGAAGGATGAATTATTTATTTACATAACTTGGGA
GAGAAATAAAATCGGAGTAAAAGAGATAGAATTATAGATACAATAAGTGATACAATTGAA
AAATAAAATGTTTGATTGAAGGTGAGACAAAAATATAATTTATTTCTAAATAATTGATAA
CGTGTTTTCTAGTTCCTTCAATTTTAAAAAGAATTGTTGATTTTTTTTTATGATACACTT
TATAATTTTTATAAAGTATTAGTTTTATTTTTTATTTTTTTATCTTTATCAAAGATTGTG
AAAATATGTATTTCTCTCTTATATATTTTAGATTGATAGATTGTCATTTATTATTTTTAA
TTAGTATTGATATGTAAGTTTTTATATGAAATGATATTTTTATCTCTCTAATAAAAAATT
TAATAATAATTAAAAGTTAAAATTGATAATTATCATTTTATTTTATAAACTCCATAAAAT
TAACATAATTATAATTATTTATATTGATTCATTGAATCGATTAAAAATATTTCATAATAT
GTAGATGAAAGAGTATAATTTTTTTAATATATTTTTTATTATTGACTAAAAATCATTGCA
TTTTTTTTCTCTTTCTTTCTCTTCCTTCCATCCATAAGTGGATATTAGAAGAAAAAAAAT
TTCCCATAAAAGATCATTGAATAAGAAAGAGTACCAACAATTTGCTTTATTTTTAATGAA
CTTCATTTAATACAAAAGTCTAAATAGGGGCATGATTATAAATTTACAGCATTTCTATAT
TGGTTGAAAAGTCAACCTTTCATGTGCAAACAATCTCTTATCTATTATTATATAAGAGTC
CTAAGGCAGCAATACACAATATTGCAATCACAACTCCAATCCTATTTTCTTCCTCAAAAA
PIGBP1 promoter1
>psP. lunatus_PI08G0000035500.v1
SEQ ID NO: 124
TATTGGACCCTAATGGCCCATTCTCTAAAAGTCCATTTGTTTTAACTTAAGCCCATTGTT
AATTACTAACTAAACCTTCTCTAATAATTAAAATACTTATTATTAAATCTAATCTTGGGA
TGTTACAGCGTGACTGACAAAAAAAAAAACATATAGGGAGAGGAGCATGTCAGGAAGTTT
CCCATATGCATCAATCATAATATCACAGATAAAATATCCTCAAACTTTTCTAACTTTCTT
CTCAATAATTGGTCTGCAACATTCAACTCTGTATTTCTAATGAAAGTTTTTCATCATGCA
CACAGTTTCCGTGAATTGAAAATGTACTACTTCACTCACCTAAATAAAATAGTTCTTTTT
CATGTGAACTCTAAGGTAACAACAGGTTCTTTTTTTTATCATTTTTTTTCTCCTTCACAG
TTCTGGATTCATAATAATGCTATATTATTTATTGTCAAATCTGATGAATCTGCACATGCT
AATCTTTTTAGTGCACAAAAATGTTATACATTCAAAGAACATATGGGTGAAGATATAGCA
TTGGAGCATAGATACCATAAGAAAATTATTGAACTCACTCACCAAACACATGGCCCCAAT
ATTTGTAGCATAGTAGCTTCTTGTAGGTATTATACATTAACTATTATTTTAGGCTTTTGG
GTCATACATGGCTGCCCAAATTATTCATAAACTTGACTTGTGCTCCACTTCCAGGATCTT
GACAGCGTATACAAAATTTCATAATATGTATACTAATTGAGTTATGATATCTTCAGAATA
TAATAGTGATAGCCTGTTATATTTTGAAATGTATTAGCATATTAAAAGGATTTAATACTG
TTTTTTTTAAAGATTAATTTCTACATTTTTTAAAAGACTAGACAACAATTACGTTAAATT
AATGAATTAAAACATATTTCATAACTTATGATTATTTTTCAATATTTTTTTTTCTGCATT
AGCTTTATAGATACTCTTTTTATATATAAAAACTAACTTTATCATATTGAAATAAATTAA
TTATCTTGTAAATTTTCTTACTTAAGATAAAACAAGTTCTGATTATAATTAGTTATAATA
ATTCATTTTTACAGCGTAAAAATAACATGTGTTCTAATTGTAAAAGCATTAGTATGTCGA
AATGCTTTAAAATTATTGAAATAAAAAATAATTAACACAATTGTTTATATCTAAAACAAA
ATTACCGAAATGTTGTGGAAGAAAGGTAATGCTGTAGAAAAAATATAGGCAATGAAAATT
ACTGAAACAAAATAACTTTTACTAATATACCAAACAAACCTAAAGAAAGAAAAAGAACAT
TCTTTGCAATTGCAAAGCAAAGACTTCGTTGCCTTTATTGAAACGTCAGTATTGACTAAA
ACTGAAAATCAAACATATATCAAAGGGAATTGCATGGCATTTAAGAAAAACTATTTTTAA
TAATAATTTTTTTATTAAAAAAACATTGCTAATTCTTTTTTGAAAAAAAATAATAATTGT
TTTGCTTTAAACTATTTAATAAACCCTAATTGTTTTGTACAATTAATTAGTTAATCAATT
CAGGGATTTTGTTTGATAAATAATAATTAAGAAAGTCCAACGGTGGTTTTTTGTTTTTGT
AGTTTGCTTTAGAATATGTGAAAGCACTTTTGTCTGTAAGTTGAACACATTTTTGTTTGT
TGTTTAACGGTCACCTGCTACGTCTTCTATAAGGGAGCGTTGTAACGTAAACCCTCGATA
TTTGATTTCGTACGGATTACGATTTTTATTTTTAATAATATTCGATATCTCTTTTAATTT
AATATTTCAAAAAAGAAATTTTCGACATTCCTTCTGATTTTTTCTTAATTTCTTAACCAT
TGTTTTAAGACTCTACTCAACCCGATTCATAATATTATATTATAATTTAACAATTAATGA
AGAGTTGAAGATTATTGAAATAATTTACACTATATTAGTTCAACGGTCATCTATATCATG
CTGCATCTTACTTACAACAACTACTTATTTATTTCTCTACACAATTTTAATTTAATTTTT
TCTTAAATAATTGTTTATAGATTTTCAAAAGAAGAATACATTTAAATTTATTGCGTTCTT
TCACCCCTCGATATATCCTCGATCTAAAATATACATAATTTTTGCAATTTCTAAACACTA
AAAATAAAATAAAAACTGAAATAGCGACATTAAATCTACAATTCCAGAAAAAAAAAAAAA
CTTAACCCTAGCTAATGAACATAATTTTGATAAGAACAATTTAAGATGATCAGATAAAAG
TTTACAATGATTAGAAGAAGAAAAAGTAATTCAATAACACTTTTAGCTGATATCTAAATA
GTTCATTATGAGTAACCAATTATGTAATGTATTATACTTTAGAATAATAGTTAGATAAAA
ACGAGTCATTCTAAATCCTCTAATTATTTATTCCTGCTACTTTTGGTATAGTAGATAAAT
AAATATATTGAACCATTTTATGTTTTCAAGATGTTAACCATATTTTATATTTATTATTGG
TTATAAACATTAAGATGATTTATACAAGAATATAACATTATTCATAATAAGAGCTTATAG
TGCGATCCATTAAGAAAAACTCTTAAAATATGCCATATTTTGAATATTAAACAATACATT
GAAAACTGGAGAGAGAGAAAGAGGTTTAACAAAGCGAAGAAAAAAAGTTTCCCAGAAGCC
TGAAAGCCATCCATCCATAATCCATATACTCCAACCAAACCGCAGCCAAACACAAACCAA
CAAATGAAAAAAATATACAACTATTATAATTTATGAATTAAATATCTTGTTTACACTTCA
TAGAATTATTAACTTTGCTTCTCATATATATATATATATATTTATTAACTTTGCTTCTCA
TATATATATATATATTTATTTTTGTTCAACATTAAAATGAGAATTTTTTGTATTGACAGT
CATATACAAATTCTGCAATTTTATAAGAGAGAAAAGGCTATTTAAGATTAAATTATAAAT
GTATAATTTACCTACTCGACACTCAATTACATAGGTTTCAAGTGTGAGAAGATAAATGTT
GTTTTAGCATTGCTTAACCAAACACAAGGATTGTGATGTAATAATATAAGAAAAGCATTA
TTCTAAAGTCAAGTTGGGAGGTGAAAGAAGTCTCTGAGTTTGGTTCATATAAGAGACAGC
AAGCATTGCAGGAAGCAATCACAATTCCCCTCCCCTTCTCTTCCTCACAAACA
PIGBP1 promoter2
>psP. lunatus_PI08G0000035600.v1
SEQ ID NO: 125
TACATAATTATTATATTTCTATTAAAGACACCCTTCTAATTTGATCCAATTTGACCTGGA
TTGATAACGTAATTACACAAATTTTATGATCATATAAGATGGTCCCATTATCGTAAATGT
TTTGTTCAGCCCAATTCATTAGCCCACTTAAGACCTCCACATTATCGTAAATTGCATGTT
ATTTATCTTTTCCTTTACCGTCTATTCCATCCAATTATCTTTAGTATCATACCATTGTGA
CTTAGCTTCCACACCTATCTTGCTTGTCATAAATGAATCTTCAAACTCCATTGTTTCATA
CCTATGGTCTATCTTTATATTTGCATACAATTTGGTCATCGTCCTCCATGATGATAACTC
CATTAAAGATTTTTTTCTATGAGAAACTTGATATGGGATTGATGTCCTAATGAATACTCC
CTCAACTCTAATTTTTTTAGGAATTTATTGTGTTTTTCTGTATAAAAGAATGTACATCGT
AATGTTTACCAATTTTTTTATGCATTCAACACTCTTGCACAAAGGAATATTGTAAGACTA
TACACATACTAATTTATTACCTAATAACAATTATGAGTTTCACTTCTAAATATTATTGAA
CATGTCCCCAACCCCTTTACTAAACAACATGTCATTTATACAATTTTCTTGTGTTCCAAA
AATCAAAGTTGGATTGTCCTTAAAATAAGACAATATAATTCCTCTCATTTTCCATAAATC
TCCTCCAATACTAGGTAGATTAGTTTATTAAACATTGTTAGGTCATTGAGCCTTCTCGTC
TAGCTTTTTGTAAGCCATTTTTCTTCTCCCATGTCTGAATTTATTATCATATCATTACAT
TTCTCAACATGGTCTTGAAGTCTCATCCTAGACTTCCATTTTTGTGTAGTAGCTTTTGAC
TTCACCACTTCCACATATGCAAGTTTGTCAGTCATGTATTGTCTTCCATGTTCTATTATA
CTTTGTTTTTTCCCTTATTTTGATGCATGTGAAGTTTTTGTCTTTCAATTTGTATTATTT
GTACCCTTTTATCCTTAAGGATGTTAAAAAACATTTCATATTCCCAATCATGACATGATT
AAGTTCTTTTTCCATTTGTCTTTCATCTCTCACATCCTTAAACCTTAAAAATTCAAATCT
TTGTCCAACTCTATTCCTTCACCTAGATATGAATATCTCTCAAACTCACCTCATCTCAAA
AATATCTTTCACAAATCTTCTTCATTGTAGGCGCCTAGGAACCTATAAAAGAAGAAGGTA
GAGATGTTTTTCCAAACTCACCTTATCTCAAGAATATTTTCTATAGATCGTTTTCATTCT
AAGCACTTGAGAACTTATATATAAAACAAGAAGGTAAAGATGTTGTTTCAATAATTCTTT
CAACTTTTCTTTTCACTGGACCTCGCTTACGATCGGTTGCTCCCCATATCTTTTTTTTAT
CTAATTTATAATCAATGTGAAATATTAAATTATTTTTAGTATATCCCCTCATATCTATAA
TATTAGTGGGTTTACCACATGAATAACATAATAAGTGGTTTAAAAATATATTTAAATATT
TTCTTAAAATTTGAGATTGAGTTAGTTTAACTCCATACTAATAATCTGTACTAATAATCT
GTAAAGTGTTTAATATTCTATATCTAGTTATCTTCATGAGAATTTGTATTTAAGATCTCT
TGCCAACTAAAAATAATATCAATATTTATAATTTTCATCTTATAAATTTATTTTGTGAAA
TTTAACTAAATTCAAATTTATGTTTTTAAGATAATGTTAAAATCTTTTCTATACATTGTT
TATTGTTGTGTCAAATTACATACAAATAGTTAATTTTGCAATCTTTATGCTCAATAAATC
TTATCCTGAATTTAAGAAGGTGTTACATGTCCAACTTAGACGGCTATATAACAAATATAT
TACTTACAAGATAATAATTATCTAATAAATTAATGGATTGAATGAGATTTAAAATTGATC
TCTAAGTTTTTTTTTTTTTGTCTTACTAATTATTTAAAACCTCTTTATTGATTCATCAAC
AATCTACTAAAAAATGCAGTCTTTGTAAACTTGACAAATTTTTAATCGTGTAAAAAAATG
TGATAATATCATATGCTGAATGTCAAACTTGACCTATACTTTAGATATGATTATCATACA
TCTAGTTGTTCTTTCATCTTCTTTATTATATCTATTAATTGTTTTTCTCTCTTTATCTTG
CTTGCTTCCACTTATAGACTCTAAAATGACATTCAGCATTCAGCATCTTTCCGTAACGAT
TAAAAGAATAAGAAATAATAAGATCAACACATTATTTTATTTTTAGTGAAACTATTTTAA
TAATAAACACTGCACAGTGGCAGCCGGGCAAGATTATAAATTTGGGAAGTCAAGATTTAT
TATCCCTTCTTACAACTACTTTTATTAATCTCTTTAATTAATATTTCAATTAAATAACTT
AATTCAATATTTTATTAGTATATAGAATTATTAAATGGTTGTAAATGCATGCTTGAAAAT
AGTTTTAGTTTTTTCTCTAAGGGAAAAATTATTTCAACACCATTTTTTATAAATATATCC
ATATCAAAACTGTGATTAAAATAATAAAATATTAAATTAATATATTTAACTCATGATACG
TTTACACCACTCTTTCTTTCTTAACGTTCATCTTATAATTGTATATATTATTATTATTTT
ATTGTAATATAGTATTTTATTAGAAAAATCAGTTAAAAGTAATGTATTTAATGAATAAGA
TATTTTTCCTTAACTAAGACATAAATTAAATTTTAATAAATTATTATTATTAAGAGTTGT
TAGGAAGTGGTAGAAGTTTATAAAGAAGTTTTAAGTATCACTCTTCTTTTCTTAATTATA
CCTTAGAATTTCTCGTTTATAAAGTCAACCAATAGGCCAAACAAACTCTCATCTCTTTGC
TTATAAAGTGGCTTAGGCCGCGGCACCACACAACACTGCAAGAAGCATTCACAACCTACA
PIGBP1 promoter3
>psP. lunatus_PI04G0000054600.v1
SEQ ID NO: 126
GGAATGATTGTCCCACCATTTCCTACCCTAATAAAGAATATAAGAGCAAGGGATACCCCT
CCAAATCAAGAAATGAAAGGTCACGAGAAAGGAATAGGAAAAAAGAAAAAGAAAAAGCAA
AAAGAAAAAAGAAAAGGAAAATTCAAAACTAGTGAACTATATAAAGAAGAGATATTTAGT
GCTTTAATTGTCTTGGTAGAAGGTACTATGATGTTAAATGTCTCAATAGAAGAACAGTGT
TTTTAAAAGACCAAAAGATTGAAAGTCAGGATGAAGCTCAATCTTCACCTAGTGAAGATG
AAACTCTAGATTCTGATGAATAGGAAGCTATACCTTGTGAATGAGACTTGTAAATGGTGA
GAAGACTTATTGAAAGCTTATCCATAGAACTTGAACCATTTTAAAGAGGAAACATATTCC
ACACCAAATGTAAAGTCTTTGTAAAAACTTGTTATTTGATTATGGATAATGGTTTTTTGC
TGTAATTATTGTAGTTCTAGATTGGTAGACGAGCTAGCCTTAACCGCTACATCCCATCCA
AAACCTTACAAACTTCAACGGATAAAGATGATGGTGGTGTAGTAGTTAATTAACAAGTGA
GTATCTTAATTATATAAGTTTAATGTGACATAGTCTCTATGGAAGCTTGGCATATTCTAA
TTGGTAAACCATGAAAATTTGATAAACAATCTATCCATAATGTTCTTGTCAATAAAATAA
GTTTCTCTCACAAGGGCAAAAGGATAGTTGTGTATCCTCCCACACCTTAACAAGTGAGAG
AGGACCAAATAAAAATGAAAAAAAAAATAACTTGAAGAGGAGAAAAGATAGAGTAAATAA
CTAAGTCAGAAAATCTCTCTCACACTTAAAATAGATGGTGAGAAGGAATTGAGTTTGGAG
GTTTGTGTCCCTCCAAAGAAGTTGTTAAACAAAATTTTTTGAAAAATAAAATAATAAGTC
TCTTCTAGTTGAACAACCTATTTTTCTTTTCTATTGCAAAGAGATACTTGTTACCACAAG
TCGTGAACTTGATTCTCTACCACATATATAGGGTTCAACAACCTTAAACAATTTGGTGAT
TTTTTCTCAAGTAGGTTCCTCATGGATTTCTACCTTTAAGGGAATATAATATCAAATAGA
CTTAGTTTTTGGAGTAGACTACCCAATAGGCCAACTTATAGGACTAATCCATAAGAAACT
AAAGAGATGGAAAATCAAGTAAGTGACTTGTTAAAAAAGGATTGAGTAAAAAGGGTCTGA
ATCCTTGTGTTGTATCAATATTTTTGGTCCTTAAGAAGGATGGGTAAAGGAGATTGTAGG
ATCATTAATCACATCACTATAAAATACATGTACCAATTGCTAGGTTAGATGACATGAAAT
TAACATATTTTCCAAAATTAATCTTAAAAGTAGTTATTATCAAATAAAAATAAAAGGAAA
TGAATTGAAAACTATTTTTAAGATCTAGTTTGAATTGTACAAATGGTTAGTCATGCATTT
TAATTTAACCAATGTTCCTATAATCTTTATGAGATTAATGAATTATCATGTAATTAGCGA
TTTCATAAAAATATTTGTGGTACTCTACTTTAATGACCTCTTAATCTATAGTAATTTAAA
TAAGAACTAAAATAAATCTTGTTGACTCCATGAATTTTGAATTCTTCTACTCAACTTAAG
CAATATTTTAGAACTGCAATAAACGTAAGTAGAACTAACTTACTACTAATTTCTCTTAAT
TACTAATCTATTTTAAAATAAATATATAAAATAAGTTATAAAAAAGAGTTTTAGAAGATT
GGTATAGTTTTACACCCATGTTCAAAATAGAAAAGAGAATTCCTTCCTACACCCCATTAA
TTCTAACTTGCACCCAATGAAAATTTGGGATAGATTTTGTGTTATGAAAATGAAATTTTG
AAATAGAATTAAAAAAACTAAAATATCATTATGAAAAATCAAATCCAAAATACAAGTAAT
GCACGAAAGAAATTTTTAAAAGAAAACAAAATCTAAAAAATATTTTAAAAAACAAAAATA
TGAAAACATATCTTGAAAAAACTATTCTGGAAAGTTAAATTCAACAAAGTATTCTAAAAA
CTCAAATCTAATAAAAGCCTATAATACATAAACTCAAATCTAGAAAGTAACAATTAAGGC
AATTTAATTACATAGACTTTGGGAACACAAAATCTGAAGTTGTTTGAAGCCTACAATTGA
AGTCTCTTTCCCTACATCATAAATCATAGTAATTCCTTAACCGCGTAAAGTTCCTTAGAT
TGTGTCTTTCCTTCACACTAGGACAAGACATGCCCCATAAATTCAAACTGCCATATCATG
ACGCCATCCCTTCCTTGCACTTTCCTAATCAGAAACCTTCTCACTTCCTTTCTTCACACA
ATCTAATTCTCTAACCTTCCCAACCCAACAACAACATACCCTAATTCCTTCACCATTCAA
CCTTCATAAACCCCCTCTTACCCATCTCATTCAATCATATCCATTCCAATCCAATTCCTC
TCTTCTCTATCCCATGCAAAATCATAACCCAATAAAACCATTCTCCTTGTCAAACCCCTT
CAACCACTCCACCCAATTTAGTGTCACAATAATCACACTTTCTTGCTTAACCAATTCTCC
TTACTCTCAATACCTGAATTTTCCCTAGGATCAAGGTTCATACTTATCATACCAAGGTTA
CAGTTTTTTTTTTTTTCTCTCTAGAGCTTGTCTTTCTTAAAAAATATATTTACTTTTTAA
TATAGGAATTTGTAGTTTATGATTACTTTTTTTAAAAAAGAATTCTTTTTTAGCCTCATT
TGTTCACGTGTAAAAGTTGTTTTTTTTTTCATTTAATGAAATTTTTTTATCTAAAAATCA
CATTTTTCAGAATTTTTTCTAAAAGAAATTCACATATAATGAAAAATAATCTAACTTTCA
CACAAACGCGTACTTATATCAACTTTAAAAATTAAAATAAGCTTATTAACATTGTCCTTA
TTAGAATTTTGCAAGTAGTCTTCTAATAATTAATTTTTCTTTAATACTAAAATATTAAGA
AAATAATATTGCAAACATAGATTTAGTCAACAACCTAGAAA
PIGBP1 promoter4
>psP. lunatus_PI04G0000054700.v1
SEQ ID NO: 127
TATATATATATATATATATATATATTATTTGTATTTTTTTTAATATATTTTATTTATCTT
CATAGTATTTCCTGAGTTAATTATTAAAGTAAGAGTTTAGTAAATAGGAACTTCAGTTGC
TATATTGATAAATTTTAAAAAATAATTTAAGAATATTTGTTTAGCGATAAAATTTATAAA
TTATAAATTGAATAATTCAAACTCAATTTGAATTACTTATGATTAAATAAATGAGTAAGT
TGGAGTTTTTAATTTTTAACAGTTTTTTTTATCAAATAATAACTGTTTTTTATTAATTAA
AACATAATTATTAAAAACATTTCTAAGTCTCCAAATTATGTTTAAATCCAATCAACACCA
AATAAAAAATATAAAAGTTTAAATCTAAATAAAAAGGAAGTGTATTGAATATTATATTAC
ATTTTGAAATTTTATTCTTTATTTACCATACAACAAACACCATAAGTACTAAATAAATAA
CATTTAAATATATTAAATGTTTTAACTTAAATTGACTTAAATAAATTTTAAGACTTTGAT
ATTTATTTTGAATACTTAATATTTGTTCAAATTAGGTATGTAATGAATTAAACATTTTAG
TTTTATTTTATTTTATTTCTTATATCAATTCTACATTCACATATTTTTTAATTAAATATT
ATGTATTAAGTTATTTTTGCTATATGTATAAATAATACACATTATCATAAGATAAAAATG
TTTAGATTGTTATGATAAAAATAAATAAAAAATCAAAAGAGAAATTTAAATATATTTTAG
AGATTAATTAAAACATATAACATATTAAGAACAAATTATAAATAAAATTTTATTTATAGA
GTTAAAATAATATTTTATAATTTATTAAGTACTTAATTTAGAAAAATAATTTGTTGGGTA
TTTTAATAAATATAATATATATAATATATTAGGGACTCAATTTATATAAGTAATTTAAAT
TTTAAAAAAATTATATGATTGACCTGATTTGCAGGCACGGTTTTAGCAGGAACTTAAAAG
CAAATTTTTAGAAACAACAGCGTTTTTTTCCCCCTTCTTTCTACTGATTTCTTTCGGCCC
TTGTCCACCTTCATCATGGCTTTCCATGAATCTACACAAATTAGGTTGTTTCTTTCTGCA
TCTTTATAATTTTGTGACACTCTATGCTTTTTAAAATATAAAAAATAATTTTTATTTTAT
TGATATTTTTCATTTCAAAATAAATGATTCAGATAATTCTGGATTATAAAATTTGATTCC
AGATTCTGTAATGTAGAATGAGTTTCTTTAATACAATACATTTCAAATTATGAAATGTAG
AAGATATTTTCGTATTATAAAATTTGAGAAGTATTTTTAAATTTGAGAATACTTTTTGGA
TTTTATAATCTGAAATGCATTTTTTTTTAAAAAAATATTATAAATTATAAAATCTGAAAG
ATATTTATAAATTTAAAATACATTTTAGATTTTACAATGCGAAAAGTGTAAATATGAAAA
AATACATTTTACAGATAATAGAATCTAAAATATATATTTTTTTTTTAAAAAATAGTCTTT
TCGCTAACCTATGCAGTGTTAGAAGAAGTTATGGAAGTGTAGAAAGAAACAACCCACAAA
GTAATTGAAAAATCCAAATGGCTTAAGCTTAAAATTTTGAGATATTTCAAAATTTGAAAA
CATATTTTAGAATGGAGATTCTGAAATATATTTTTGAATCCAAAAATATATTATGAATTT
TCATATTAAGTTATTCAAAATATATTTTTAAATCTAAAAAGAATTATGAATTTTTAAATT
ATATAATTTAAAATGCATTTTTATATTCAATAATATGAAAATATATCTAAAGTATAATTT
AGAATATATTTTTATATTATATAATTTTTAATAATATTTTTTTTATTGTACAAAGTAAGA
TGTATTTTTTATTAATGAAATCTCGAAATATATTGATATTGTATAATCTAAAATGTATTT
TTAGAGTCTACAATCCAAAATATATTTTATATTGTATAATTTGAAAAATATTTAAGATTA
TAAAATTTAAAATACATTTTCATATAAAAAAAATTATTATGATTTATTATATTCTAGATT
AAATAATTTAGAATATTTTTTTAATTATGTAATCAAGAATCATTTTAATTATACATTACT
CAATTTAGAATAAATTTTTACATTATGTAAAATATAAGAGTTTACCAATCTATACCATTT
GAAATGAAATTTTAGATTAAACAACTTAGAATAATGACTCATAAATAATTTGAACTTTTC
TCACAATAGCTAAAAATATATGGAATGAAGAAAGAAGTTCCCTTGCATCAATAGGTTTAT
AAGACAATAAAGTGTTGGTAGGTGTTAGAGATATTTTTTTTAAAGGGGTGTGATTTCATT
GTAGAAAGCAATACATAAATTGTATCAATCATCATAGTGTGAAGTGATGGAACTCTGGTT
TTTAATTTACCTCTAGGTGAAGTAATGCATGGATAATTTAGTTGCATAGGAAAATAAATC
TCTCAAACTCTATTTAGAATTACTGTTCTTAAAAATTGTGAGAAAGTAATATGATTCAGT
TTGATTTTTTAAAATTTTAGTATTTTGACTCTAAATCATAATTGTATTGTGAATGATATA
CAAGATAATCTACTACTAAAAGTAGCTTCTCCATAAATAAAAAAAAATTAAGGAGAAAAA
AAAATTCTTTGAACAAGTTAAAGAAAGAAAAATAATTCTAACACTCTAATAATAAAAAAC
CTTTGATCCTTACCCCATTTTCTGCACTCAAGCCAAAGAATGGAATCATTTCTTCCATTT
TTCTGTTTCTATGAACATTCTTCCATTCTCTTCAAACAATCCCTTTCTTCATAAATTTCC
AAATTCCTTGCTTTTCTTTTCATCTTCATTTTTCTCTTCTACATTAAGTACAGTAACCAA
CCCTTTCTATTTCCATATCCAACTAACTTAACGTCTTCCAAATCTAAATCCAAACTCACC
PIGBP1 promoter5
>psP. lunatus_PI04G0000054500.v1
SEQ ID NO: 128
GATCCTTTCTAGTATGGATGATCAGGTTATGCCCAAGTAGATGCAAGGGTCCCTAATTAT
GGTTTATAAATTGTTTATTGTTCAACACTTATAAATTATGTTGTTAGTCTCTTAATCCTG
GTAAAGAGACCTTTATTTTGTATGTTTTCGAGGTGTCAAAATACAAGGTAATATCTCGAC
ATCTCAGATACTCCAATAATACAACTATGTTACAAATAAAGAAATATCATAATTTTGTAT
TTATTATAATTACAAATAAAGCTAAAAAAAAGCATGAATAATAAACAAGTTAACTTTTAA
AATTGAGAATATTGCACATTAGAATTATATTTAGTAAATTCAAATATAAACTAAAATATC
TATAACAAAAAATTAAACCAATGAACCATAAACTAAATTTTCTATTATAAAGAATAGGAA
ATTCAACTAGTAAGAGTTGTTACAAAATTTTAATATTTTTAGTTTCACATTTTTTCTCGT
GACCTATTTTAAATAATTATAGTAATGATGATTTGATTGTTCTAATATTTTTAATCGTCT
TACATTGTTTAGAAGTTGAGAACAAATATAATTTAAATACATTTTCCTTCCTTAACTCTC
TAAGGTGCTTTTTAAGGATAAAATTGTGATAGAACTTCGAGTTTCAAAATAGACAATACC
TCAGACACAATTATTGACCCTAACAAAGTTACCTCAACAGACCTGAGGTTGGAACTCTTA
TTTTCCTTAGACGTTGATGAGAGACTTGTTGTGATATGTCTGATAGTCCTATATTGCTTA
GAAGTCAAGTTATAAATAATTTAAATATATCTTCATTCCTTAACATTTTAAAACGCCTTT
TAAGATAATAAAATTATGATGAAACTTCAAGTTCTAAAATGCACAATATCACAAACAAAT
ATGATGATTGATTCTAATAATTTTATTTCAATAAACTTTTATACAACTCTTACCAGTCAA
GCTCCTTTCTAATAACAACAATAATGTATTATTTTTTCTTTCATATGTAGTTAGTCCTCG
GAGAGGATTTAAAAAATTTTAAAATAATAATAATGGTAACAATAATAATAATTAATTGTT
TTGTAATAACTAATCAATTATCTTATAAAATAATCAATTGGGATAAGTAAAGTAATTCAT
TATTTGAAGTTAGAAAAAAAGTACAATATTTGTGGAGAATATATTACGATTAAAAGTAAT
AAACTATATCATTTTTATTAAATTTGTATTTTTTTTTATTAAATTAATAAATAAAACTAA
TCAATTTACGAATCATTCTTGCTTATTTTTAATTGTTCTTCAATAACTCTGGAAGATTAA
ACGTAATTTTTGCTAAAAAATAGTAAATGATATATTGCATAACTCTTTATTTTTATCTTT
TTGGATATTACATCAAATGATATATTTCATATACTTTGTGAATTTTACAATCATTTTTTA
GGTGGTTCATGTAATAGGCATTTACATGTTGATCTAAAAAATTAAAATACTTTAAATAAA
TATTGTACTTGTTGTGTTATTTTACATTAATTAATACTTTTAACTTTTTAATTTAAACAT
TTCTTTTTCAAATATATGTTACTTATCTTCTTTACTTATTCATTGTTAATAATTATACAT
ATGTTTATTTCATTATATTAAATATTTTTTTATTTCTATTCAAAATTCTAGATAAATTAA
AAAATTACTCTTAAATATTAAATTTATCACCCTAAATATAACTCGACAATGAAAATTTCC
TTAATTGAAAATTCAAATTGAAATACATTTACAATTTGAAGGGTCCATTATCAAAATAGT
TGTTTGTGTGGAAAAGTGTGTTGTGGAGAGCACATCCTTCCTCCATGCTTCTATTTTGGA
TGTTGACTTCTTCCTCTCTTTCATACCAAAAAAGGGTAAGACACACGAAATAGAAAAAAA
AAATAATTAATTTTAGAGACATAAATGGAACTTAAAAATATTAGAAAAATTTTAAGTTTT
CTCTAATTTTAGAAATTAATTTTACAACAAAATACAATTGTGTATATGTTTACAAAATGG
GAGGTATTCTTAATTTTTTTATCTAGTCCAATCCAACTTATTTATATTTTTATGGGTTGG
ATTGAATTGTTGTGTGTCCCAATAAAATTGGATTCCCTAACATGTTTATAAATTTTTAAT
ACGAAAAACTCAACTTCATAGAGGATACTAGGTATTATTTGTGTATTTTTTAAAATTACT
CAATTCATTATCAATCCGATTATAATACGTTAGATCGGTACTCAATAAATACATAAATAA
ATAAATTGGATGGGATGCAATTAAATTTAATTAAATAAAAATCAAATTAAACTCACATCT
CAACGTGTTGAGTAGTATATATCATAGACGAGAAGGAAAATTCATACATGCCAAGACTTT
TCCAAACAATAATATATTTTGGCTTGCTATAAAAACTTCCACATGCAATATGATCCATAT
GTGTATTGATCCATAACTTGTGTTTATCTTGCTCAAATTCTCCATTTGTGTGCACATAAA
TGTGCTTTTATGTGGGGTACTGGTTTTTTTATTTTTGCATCTGAATTTGGTATGTTCCCA
TTGAGAAACTTCTGTTTTGCTTCTTGACATGGTCACATTCACTCACCTTTCTGCCCAAGT
TTCACTTCCTTTCAATCAATCATTCAATCCTCTTTACTCACACCCTTCCACAATTCATTC
ACCAGAAATTTAGAATCATTCATACTCTCTAACATATCATCATCAACACACCAAAACAAA
AATTTAACCTCAACTTGTACTAAAAGCCAGTTCACCAAATCAATAAACTATGGTCAACTA
TCCAGCAAGTATATCCTCCAAAAATCCAAAATCCAAACACCTCTTTCCTACTCATAACAA
TTTAATAATTAACCAGTTGTTGATAGAGTAGCCGCCAATAATGAAAAAACACCAAGTCTA
AGTATCAAGTGCATTCACCTCATCCTCTCCAAAACCCATATATACATCACACCATTCAAG
CATTCATCATCACCACAACCAAACACCACTTCCACCACCTTCAGAAGCAGCA
PaGBP1 promoter1
>prP. acutifolius_Phacu.WLD.008G033800
SEQ ID NO: 129
TTGTTTTAAGTTAAGCCCATTGTTAATTACTATCTAAACCTTCTCTAATAATTAAAATAG
TTACTAAATCTAATATTGGGATGTTACAGCGTGACTATGACCAAAAAAGCATATGAGGAA
GTTTCCCATATGCATCAATCATAATATCACAGATACAATATTCTCAAACTTTTCTAACTT
TCTTCTCAATAATTGGTCTGCAACATTCAACTGTGTTCTAATGAAAGTTTTTCATCATCA
TGCACACAGTTTCCGTGAATTGAAAATGTACTACTCCACTCACCTAAATAAAATAGTTCT
TTTTCATGTGAACTCTAAGGTAACAACAAGTTCTTTTTTTATCATTTTTTTCTCCTTCAC
AGTTCTGGATTCATAATAATGCTATATTATTTATTGTCAAATCTGATGAATCTGCACATG
CTAATCTTTTTAGTTCACAAAAATGTCATACATGAACTATTATTTTTGGCTTTTGGGTCA
GACATGGCTGCTCAAATTATTCACAAGCTTGACTTATGCTCCAATTCCAGGATCTGGACA
GCTTATACACAATTTCATAATTTAATACTGTTTTTTTTAAAGATTAATTTCTTCATTTTT
AAAAGACTAGACAACAATTACATTAAATTAATGAATTAAAACATATTTCAATAACTTGTG
ATTATTTTTCAATAACTTTTTTCTGCATTAGCTTTATAGATACTCCTTTTATATATAAAA
ACTAACTTTTTCATATTGAAATAAATTAATTATCTTGTAAATTTTTTTACTGAAGATAAA
ACGAGTATTAACTGATTATAATTAGTTATAATAATTCATTTTTACAGCGTAATATTAACA
TATACTATATATATATAGTGTTCTAATTGTCAAGCGTTAGTATGTCGAAATGCTTTAAAA
TTATTAAAATAAAAAATAATTGACACAATTGTTTATATGTAAAAAAAAAAATACTGAAAT
GTTGTGGAAGAAGGGTAATGCTGTAGAAAAAAATATACACAATAAATTACTGAAACAAAA
TAACTTTTACTAATATACCAAACAAACCTAAAGAAAGAAAAGAACACTCTTTGCAATTGC
AAAACACGTCTACTGAAAAAGCAAGACTTTGTTGCTTTTATGAAACGTCAGTATTGACTA
AAACTGAAAATCAAACATATATAAAAGGAATTACACGGAATTTAAGAAAAACTATTTTTT
TTTTAAATTTAAAATAATAGTTTATTTATTAAAAAAACATTGTTTATTATTTTTTAAAGT
GTTTCTTTTGGAAAAAAAATATAATTGTTTTGCTTTAAACTATTTCTTAACCCTAATTGT
TTTGTACAATTAATTAGTTAATCAATTCAGGGATTTTGTTTGATAAATAATAATAAAGAA
AGTTCAACGGTGGTTTTTTGTTTTTGTAGTTTGCTTTAGAATATGTAAAAGCACTTTTGT
CTGTGAGTTGAACACATTTTTGTTTGTTGTTTAACGGTCACCTGCTACGTCTTCTATAAG
GGAGCGTTGTAACGTAAACCATCGATATTGATTTCGTACGGATTACGATTTTTATTTTTA
ATAATATTCGATATCTCTTTTAATTTAATATTTCAAAAAATAAATTTTCGACATTCCTTC
TGATTTTTTCTTAATTTCTTAACCAGTGTTTTAAGACACTACTCAACCCCATTCATAATA
ATTTAACAATTAATGAAGAGTTGAAGATTATTGGAATAATTTACATTATATTAGTTCAAC
GGTCATATACATCATGCTGCATCTTACTTACAACAACTACTTATTTATTTCTCTCTACAC
AATTTTAATTTGATTTTTACTTAAATAATTGTTTATAGATTTTCAAAGAAGAATACATTT
AAATATATTTTTGCTACCCTTGAATTTTTTTTATTTTCGTCTAGAATTTATTTTCTAGTC
CTCACATTCAATACAATTATTGAGTTTTTTTTATCCTTCAATTTATCCTCAAGTCGATCA
AAATATACATAATTTTTGCAATTTCTAAGCACTAGAAATAAAATAAAGACTGAAATAGCA
ACATTAAATCTACAACCACAGAAAAAAATACTTAAGAACAATTTATGATGATCAGATAAA
AGTTTACAATGATTAGAAAAAAATAATAATAATTCAATCACACTTTTAGCTGATATCTGA
ATGGTTAATGATTAGTAACAATGATGTCATGTTTTATACTTTAGAATAATAGTTAGATAA
GAATGGGTCTTTCTAAACCCTCTAACAAGTTCTTCCTGCTACTTTTTTTTAATTGGTATA
GTAGATAAATAAATATATTGAACCATTTTATGTTTTCAAGATGTTAAATATTTTTTTATT
TAATATTGTCTATAAATATTAAGATGATTTATACAAGAATATAACATTATTCACAATAAG
ATCAGCTTATCGTGCGATCCATTAAGAAAAACTAAAAAAATATACCATATTTTGAATATT
AAACAATGCATTGAAAACGAGAGAGAGAAAGAGGTTTAACAAAGCGAAGAAAAAAAGTTT
CCCAATTTAAATAAGCTTGGAATCCATCATCCATAATTTTCCAACCAAACCGCAGCCAAA
CACAAACCAACAAATGAAAAAAACACACAACTATTATAATTTATAGAATTAAATAACTTG
TTTACACTTCATAGAATTATTAACTTTGCTTCTCATTTTTAATATTTTTAACTGAAGATT
TTTCTATTAAGTAGCATCACAATATAATTAATTAACATGCTCATTTTTTTTTTCTTTTGG
TTCAATAAATTTTACATTTTATTTTTGGTCATTTTGCGTTAAGTAACACATTAATTAATT
TTTTTTCTTTTAAACAAGATAACCGAAAATCCATCACTTAAAAAATGTTAACAAATAATA
GAATCCAAAATACAATAAAAATGATTGAAATAAAAAACAAGATGTAAAAAATTACTGGGA
ATAAAAGCAATATATATATTAACACATCACTTAATTATTAATATACAAATTCTGCAATTT
TATAAGAGAGAAAAAGCTATTTAAGCTTAAATTATAAATGTATAATTTACCAACTCGATA
CTTAATTACATAGCTTTCAAGTGTGAGAAGATAAATGTGGTTTTACCATTGCTTAACCAA
ACACAAGGATTAAAAAGCATTATTCTAAAGTCAAGTTGGGAGGTCAAAGAAGCTCATAGT
TTGGTTCATATAAGAGACAAGAAGCATTGCAGGAAGCAATATCACAATTCCCCTTCTCTT
CCTCTCAAACA
PaGBP1 promoter2
>prP. acutifolius_Phacu.WLD.008G033900_1
SEQ ID NO: 130
TACCAAATTTAGAATATGTTTTTTACAAAAAGACACAAAATGTTTTATGAATATATCATT
TTCGCCAAGACATTAAAGAGCATATCGTGGCTACTACTTTAAGTAGCAGTCCCACCTTCA
GCACATTCTCACCGATGGGTTTAAAGTCTTGTAGTTAACTATCTTTAACTTGTGCTTAGC
TTGCTTAGTTAGTTACTTGCTTTGTTTGTTAATTGCTTTGCATCGTTTTTCTTGCATAAA
ATTTGACTTTTCATTCCTCATTATGGTATAATTTACTTAGAAAAAGAATTGTGTGAAGTT
TTTGATGGATAGTTTTGGCTAAGGAAAGACTTGGTACTTAAGTCCTAGTGACTCACCTCT
TTTCCTGGAAAGCTACCTTCACAACTTTCCTCTTCTTTAATAAAATACTTTTAATCACAA
ATTTTTAGTCATGTCAACCCATCCCTCTAAATCTAATGAAAGTGATATGAAGTTCATCAA
ATCTCTTTTAAAACAACTTGCCAAGGATTTATTGGTGGACGGCTTTAGAAAGAGAGAGGC
GTCTTCATAAGGAGCCTCCCATAGAGTATTGGAATGACCTTAGGGGAGCCTAAGACATCG
CCATATTCCCTCCTACTATAATAGGGAGTTGATGGATAAGCTCCAAAGACTCCATCAAAG
AACCATGAGTGTAGAGGAGTATAGGCAAAAGATGGAGCTTTACATGATGAGAGCCTCCAT
TAGGGAGAATGAGTCCAATCCTGAGATAAGGGATAGGGAGAATGATTTGGTTCAACTTTG
CATCAATTAAAGTTGAACAACAAAATTTAAGGAAAACTTCAAGTCTTGATATCTAACTCC
TATTCCAAGAGAGATTTTAAAAGGAGGAGAGTACGTATAAGTAAACACCGAAAGAGACTC
TAAACCCTTAGGAAGATATGCTCACTCCACCCATTTAAATGTTTTGGAAGAGGACATCTG
CAAGCTCAATGTCCCAACCAAAGAACCTTGTTTTTAAAAGGAATAGATGAATATACTAGT
GGTGATGACAAACCTAGTGAGAAAGAAAAGATGAGAGATGAAGAAAGAGTGTATCCTTTA
GAGGGAAATTATTAAAGTAATTGAATATAATTGCGTACAATATCTTGTTTTGTAACTTCC
ACTTTTTATTTATGTTGTTTTAGCTATGTCTATATCCAAATTTCTAATAATAAGATTCTA
TCATTATTAAATTAATTGCATATAATTGCGTACAATAACATACAATAATTGCATACAATA
AGTACATATAATTTAATAACAGAGTGTTTTAAAAATATATTATTGTCATCTAATATACCT
ATTACATACATCCTATTTGTTTCTGAAAAATAAAATAAACTTTTGTAACTAAAATGTAAT
AAAAAAACAAAACTGTGTCTAGTAATATTTGTTTTAACTACCAAATTTATAATGTTTTTT
TTTTTACAAAAACATAAGATGTTTTAAGAAGATGTCATATCAAGACATTAAAGAGCATAT
CGTGACTATTACGTTAAGTAATAGTTTCACCTTAAGCACATTCTCACTTATGAGTTTACG
CTTTTCTGCTCACTTATGAGTTTACGCTTTTCTGCGATTTCGATCTCCTTCAAATCTACA
AAGGATTTCACCTAGACATCATACAAATAAATCAACTTTCACTTAAATAAACTTTCGTAA
TTTCTCATTTTTGTTTATGTTGCTTTAACTATACTCACTCTCACAATAGTTTATTGATTA
AATTCACTTAACAATTACTTTAGTGATTATCTAACTCAATATATTTAATATCTTGAAATG
ATATCGTTTATGTAACTTTATGTAATCTCGAATATTTTGATTTTGAGCTGTACAATTTCT
TTTAAAAAAAGATTTTAAATCATTTTCTCTTCAATTATCTTGTGTTTTTGTTAAAAACAT
AAATAATTATAAATTTCATGTCACAACTATTTTATTCTTCTTATAAATTTTAGAAATTTT
GTTAAGTTTGATTTTAATTTCTATAAAAATCAAAGTTTGTTTTTATTTCTTATAATGTCC
ATGTTATTTTAATTATAGTCTCTTTAAATTATTTTTATTTCAATTTAATGTTTTCCGTTT
TAACATTAAACAGTGTACGCATTTTCATACTATTTAATAATCTATTCCTTTCCTTTTTAT
TAATCTATTAATTTATTTATTTGTTTATTAATTTTATTTTATAATTTATGAATTAATTTA
TTTAGTAATTCATTTAATTGATGAATGCACAATACTAATTTATGTTTAAAAACTTAAGCT
TCTTTATAAGAAAAAAAGTATGTATAGAAATTTGGTGGAAGGACCCGAATTTTAAATTTC
TTTGAAAAACAAATTTTTGTGTGGATTTAGATGGTAAAATGGACTTTGGCCCGCTAACCT
GCCAAATTTTGATATGCTGTTTTTAACCTTCTAACATAACTTGTCTCCTCTAACTTAGCT
CATTTTTTAAATTAAAAAAAAAAAAAAAAAATTTATACTATATTATTTAGAGAATGCAAA
TTTATAATAACAATATAACTTAAATCATAAACACTTCTATAAATTAATTTTAGTTATTAA
ATATAATAGATTACTTTAACATTAAAGTAATGATTATTTTAATTTATCCTCAATTAAAGT
TTTTCAAATATTTATATAATTAAATATTTTTTAATATGTATATATAACAAAATTTAAAAA
TACAAAAACTATCTCACTATTAATCTCATTTATTAATCTGTTCCTTTCCTTTTTGTTGAT
TTTGTATGTCTATATATATATATATATATAAAGAATTTTCTTTGATCTCCTCTATACACC
ACCATTTGTTTCTTTTTTATAAGTGCAATTTAGTAATAAACATGGCAAATTACATTTTCT
ATTTATATATATAGCAATATTTATTGAGATTCTAAATTCAATCTTCATGTTCTAAACAAT
CTCTTGTATATTAAGGAGGTAACACTGAACATTGCATAAGGATCAATCATTCCCTTGCTC
TTCCATACACA
PaGBP1 promoter3
>prP. acutifolius_Phacu.WLD.008G033900_2
SEQ ID NO: 131
GAACTCATGATACACATAAGTTTGAACAATAAAATGTTTCAATTCAATAATTGAGGTTGC
AGCAACATATGCATGGAACCTTGAATCTCATATCAGTTATGTTACTTTGTTATTATGGAT
ATTTAAATTTGTTCTGGAAAATAAAATATTTTATATATAGACTTTGCTCCTTATTACAGT
GAAGTTCAACTCTTGTTTAAGATTTTTAGTATGGTCTATAACATCGATTTCAGCCACAAT
AACATCTTTAGATTATTATTACTCTATCACAAAATAGAAAAGAGATCATGTTGAAGATAA
AAAAAATAAAAGCATTTACTTGATTTTATCATTTATATGAAGGCATGTTTAAGGTGGTCT
CAAATGCATCAATCTTTAGAGAACTTTAAAAGACTATTTGAAGTCTTTTTTAAGGTATTA
TAACAATGTTGTGTATAGAAAATCTTGAAAAGTTTGAATTCTTGTGTATGAAAAAGCTTG
AAGCAGTTTCGAATTTTAACACGAAGGTATAAGATTTTTTCGAACATGGCATGTGATGGG
GATAAGATGATAAAGGACTGTGTATATTGAAGGGCAGTTTTTGATTTATTTTTTTTTCTA
TATTTGATAAATCATCTTTATAGACATTATTAAGAAATTATTTCTCGAGTTAGGAAAGAT
GGTCAAATAAATCCATTTTCATAATTATAATTCTCGAACAATTAACGTTATTATAAGTAA
AGTTAAGTCACAATTCGTTTTCGTCACAAATATATCAATATTTTATTTTTTTAATACATA
TATAACTTTTACTTATAATAGCAGGTCCTCTTCATAAAATATTTCAATAATATATTTTAC
TTTTTAAATCACATGATTAATTCATTTATTAAATTTTTTTCTAATTACTTCTTGCAAAAA
TATTTTTTGTGAAAAAAATCTTAATGTATGTTTGTTTCTCTTTGTATAAATTTCCAATCA
TCAAATTGAATAAAGTTCAAATATGCACTTAATTGTTTTTGGGTCCGAAAAAGAGAAACA
AAAGGCTTTCATAATTGAATAAAGTTCAAATATGCACTTACTTGTTTTTTATTCCGAAAA
AGAGAAACAAAAGGCTTTCATTGTACTAATAAATATTTTTATATGATTTCCTAAGCCAAC
TTATATGTTATATAATAAAAGATGTTTTTCAAAATATATATGCACCTAAACCCTAAATAC
TATTTACACTTAAATATTTTATTATTAAAATAAAAATATATATAAAAAAATATTCAGAAT
TTATGTTAAAAGAATAATTGTTCTCTAGATTTTTTTCTTTTGTCTTTTTTTAACTTTTTG
AATTTCCGTCATCGACTATTAAAATTTGTTATTAGATTTTTTTTTCTTCTAACTTTTTTA
ATTTCTCTGTCATCAACGATTATTAATGATTATTGAACTTCTGACAATAAATACTACAAG
ATTGTCTAATTAAAATCTTATAACTAGAATAAGTTTAACTTTGCTCATTCTTCTTTAAAT
AATTTTTAGTTTTTAGATTTTAGTTTTTTTTAGATATTTTGCATGTAATGTTTCAAGACA
ATTACATAATCTATTATCTTTATAAAATGTTTAAAAAATATCTCCAATAAAAATATCTAA
AAGACCTTCTTCAACCATATTGTCCTTTTTATAAAATATATGAACCAATACTATTCCACA
TTGAATCCGATAACTAAAGCTGAATTGATTTGGTATGGTAACAATATATATCCATTGAGA
TCACAATTGTAGATCTAGATTTTACAAAATTTTAACATTAGAATATACTAACATAACATA
AAATATAAAATCTTGAATTATAAAAATAAAATATAATTTTTTTATCTTAAAAACATATTT
TTAATAATAGAATGCAAATTATTAAAATAAAATCCTAATATTGAAGTCAACATAACATAT
ATGTTCTCAAGAGATTAGGTATAACTTAAATTCGATTATACAATAATTTAAAACATAACA
TATGTGTTATAGAGTGTTATGTTTTAGTAAAGTAGAGTTAGATGATAATTTATGGCTCAA
TGAATTAACAGAAGTTATTAGAACATGGCACATGTAAACATTAATCGGAGGTATTTAAAA
AAGAAAAAAAAGAAAGACATTAATGAAGAATGCAATGAAAAAGTTTTGGTTCCATCGTTG
GAATTTCACATGCCTTGAAAATGAACACATGCTCTGCCTCGTAAGGGTAGGAAGCTTATG
CTATAAAATATAATCAATCATTTAGACCCTCCAACTTACTCTACAACTAAATCCAAAATT
AATAAACAAAGGTAAACCTCGATTATTATTAATAAGAAAGAATATTTACTTTTAAATATC
TATTTTATTTATCTTATTTCGTTTAATGTATGTTTAGAGTCTCGGTCAATATCATTAAAC
AAATATCCTTAATTTTCATAAACTCAATAAATGTTACTTAACACAAAATTATTAAAATAA
TTAGGTATAAAATGTTATCTAAAATCTGAAATTAACTTGATAATTTATCTTAATTTCAGT
CCAATGTTAGAGATTCATAACTTTCTATAAATGTACACATCAACCAAGATAAATAAACAC
GTTAAATAATATTTATTATTTTAATCTATCTCAAATCAAGTTTTAACTTCCTTATCAGAA
TACATTTTACAAATACCCGTCATATCCTGATCATTAGTCAGGACCAACTATAACACAAGA
AGATAAAAGAATAACATTTGACGTCCACCATTATTACTTTTGAATATATATTTTTTATTT
ATCTTACTTCATTTAATGATCTTAAAAAATTGACTTTAATTAATCTTTGAACTTTGGTTA
CACTATCAAATAATGCTTAATTTTAAGAGAATGATATTTAAGAAATATTAAAAGATTAAT
AAAATTAATGTAGTATGTAAAATTAGTTTTTTTTATGCGTATAAATGAGAATGGAAGTAC
TACTTCGAAAGTAGTATTATTTTTTGTTTTCTGTGAATCATTTATTTATTATTTAAAACT
TTCCAAAATAGCTTATTAATTTTAATTATATGCATGCTAAAAAATAAATTAATGTAATAA
ACGAGAGATATGAATGAACTATAACAAGTTATAACCATTTTAATATAAAGAAAGATATCA
ATTGTTTTTTTAATGTACGTGTTACAATTTCTTAGAATTTGAGTTTCCAATTACTCCAGT
CTTAAAAAAAACTGGTATATATATATATATATATATATATATATATATTATACTCAAAAC
ATAAAAATAACTAATAATCAAAACAATCATTTTCAAATGTCTACAACTTTCACCTATACT
TACGCTAACTTATAAAGATCTATTGTGTATAATCTACAACAATATAACATACTTCACAAA
CATTATCTTATGAAACATAATTATGAGGAAGAGTCACATTAAATAAAAGGTGAACAATGA
CATGGTTCCATTATATATGCTAATGAAGACCAACTAATTAACATATTGATTGACATTATC
TTTATAAAACTCTTCACCTTAAAAAACGCATATGTTGTATTATTTTCACAAGGTCCATGT
GACAACATATAATAGAAGATCTTTTATTTATTTTCTTAAACTTTTTTTTAGAGAATTGTT
GACACAAATGTATTTTTGGATCACACGGTGTAGAATACTATAAGCATTAATTCACCTATA
ATATGATTTCCTAATATCATAAGTTTAAACACATGAAAAAAAAAAAATACATGTTTTGTT
GATTTCTATAACTTTGGACAAAGGGCATACAAAATCCTCCTCTCCTTGGACAATGTCCAT
CCTTCTCATATTATATTTGTTCGCAGCAAGATCCATAATAGTCTCCAAATGAAAGGTTTA
ACTTTTGGTTGTGTCTTTTCCACACAAACCAACAAACCTAAACACATCCTCTTGTTGAAA
TGTTCCAATTTTTTTTATACATTTATATGTTATTTTTATATAATATATTATTCTAGGAAT
TAACATTAGCATTAAGATTATTTCTATTAGAGGAAATGTGTTTATTGAATGGTAGTAATT
AGTGCAGTCTTTAAGGTTAGATTGATGAGATCTACTTGTTATCTTCAAAATGACATTGTA
CCATTCTTGATTTCTTTGTCTATTTTCTTTGTAACTTTCTTAGGTCTATTATGGCTCTCT
CCACTTTTTCATCTTTTTCATCCTCATACACCTTTCTCCACACATGGAGTTTGTTCCTTA
TTTTCATGTGTGTATGCAAAGACCTAATGTTATCTTTCAACAAATTATCTTTTGTCCCAA
CACTATGCATAAGTGTTTTGTTTCTAAGTTTATCATATTGTTGGACTTTCGCATCTTCGT
ACTTATTTATTATATTTCTATTAAAGACACTCTTACTAATTTGGTCCAATTTGACCTAAA
TTGATAACATAATTACACAATTTTTATGTTTGGACCATACAGGATGGTTCCATTATCGTA
AATGTTTTGTTTTGTTCGGCTTAATTCATTAGTCCACTTAAGACCTCCACATTCTTGTTT
TGGCGTTCCCAATGCCATATTAACACACTTACACTAGCTTCCTCCTCACTCCTTGTAGGT
TGTTGTGTTATATCTTTTTCCTTTGTACTTTCCTTAGACGTAGCACTATGGTCTAAAGGT
TTTTGTGCTTATTGTACAATTGGTTCTTCATCGTTTCCTACCTTGTTCCTCTTCCCGCAT
GTTATTTATCTTTTCCTCCATTGTCCATTTCACCCAATTATCTTTAGTATCATACCATTG
TGGCTTAGTTTCCACACTTATCTTGTTTGCCATAGATGAACCTTCAAACTCCATTGGTTC
ATACCCTATGGTCTATCATTTGCATACAATTTGGTTATCATCCTCCATGGTGATAACTCC
ATTAAAGCTTTTTTTCTATGAGAAACTTGATATAGGATTGATGTCCTAATGAATACTCCC
TCAACTCTAATTTTTTTTGACAATTTATTGTGTTCTTCCATATAAAAGAATGTACATCGT
ATTCTTACCACTTTTTTTATGCATTTAACACTCTTGCTAAAAAAAGGAACACCGTAAAAC
TAATCACATACTAATTTATTACCTAATAACAATTATGAGTTTTACTTGTAAATATTATTG
AACATGTCCCCAACTATACCCTTTTACTAAAAAACATGTCATTTATTTTATTCTCTTGTG
ATCCAAAAATCACAAGTTTAATTGTCCTTAAAATAAGACAATATAATTCCTCTCATTTTC
CATAAATCCACTCCAATACTCGGTAGTTTAGTTTATCAAACATTGTTAGGTCTTTGAGTC
TTCTCGTCCAACTTTCTGTAAGCCATTTTTCTTCTTCCATGTCTGAATTTATTATCATAT
CATTACATTTCTCAACATGGTCTTGAAGTCTCATCCTTGACTTCCATTTTTGTGTAGTAG
CTTTTGACTTCACCACTTTCACATATGCAAGTTCATCAATCATGTGTTGTATTCCATGTT
CTATTATACTTTGCTTTACCCCTTATTTTGGTGCATGTGAAGTTATTGTCTTTCAATTTG
TATTATTTGTACCCTTTTATCCTTAAGGATGTTAAAACAAAATTCATAGATCTTTTCTCA
AACTCACCATGTCTCAAGAATATTTTCCATAGATCTTTTCACTCTAAGCATTTGAGAACT
TATAAAACAAGAAGGTAAAGATGTTGTTTCAGTAATTCTCTCAACTTTTCTTTTCGCTCG
ACCTCGCTCATAATCAATTGCTCCCCACGTCTTTTATTATCTTATTTATAATCAATGTGA
AATATTAAAAAAAAATTAATATATCCCATCATATCCTATAATATTAGTGTGAATAACATA
ATTGATTTAAAAATATATTTAAATATTTTCTTAAAATTTGAGATTAAATTTAGTTTAACT
CCATAATAATAACATTTAAAGTGTTTAATACTCTATATCTAGTTATCTTCGTGAGAATTT
GTATTTAAAATCTCATACCAATTAAAAATAATATCAATATTTATAATCTTCCTTTTATAA
ATTGATTTTGTGAGATTTAATTAAATTTATATTTATATTTTAAGATAGTGTTAAAATATT
TTCTACAGATTGTTTATTGTCGGGTCAAATTAGGTACAAAATGTTTAATTTTGCAATCTC
TATGCTCGATAAATCTTATCCTGAATTTGAGAGGGTGTTACATATCCAACTTAGACTGCT
ATATAACAAATATATTACTTACAAGATAATAATTATCTAATAAATTAATGGATTGAATGA
GCTTTAAACTTGATCTCTAAGATTTTTTTTGATCTTACTAATTATTTGAAACCTCTTTAT
TGATTCATCAACAATCTACTCACAAAATTGTAGTCTTGACAAATTTTTAATCGTGTAAAA
AAATGTGATAATGCTTATCATATGCTAATGTCAAACTTGACCTATACTTTAGATATGATT
ATCATACATCTAGTTGTTCTTTCATCTTCTTTATTATATCTATTAATTGTTTTTCTCTCT
TTATCTTGCTTGTTTCCACTTATAGACTCTAAAATGACATTCAGCATTCGTAACGATTAA
AAGAATAAGAAATAATAAGATCAACACATTATTTTATTTTTAATGAAACTATTTAATAAT
AAACACTGCACAGTGGCAGCCGGGCAAGATTATAAATTTGGGAAGTGGTGTGCTAACTTA
ACACTTCAAGATTTATTATCCCTTCTTACAACTACTTTTATTAATATTTTTAATTAATAT
TTCAATTAAATAACTTAATTCAATATTTTATTACTATATAGAATTATTAAATGGTTGTAA
ATGCATGTTTGAAAATAGTTTTTTCTCTAAGGGAAAAATTATTCTAACACCAATTTTTAT
AAATATATCCATATCAAAACTGTGATTAAAATAATAAAATATTAAATTAAGTTAAAAGTA
TTTCTTTCTTAACGTTCATCTTATAATTGTATATACTACTATTTTATTGTAATATAGTAT
TTTATTAGAAAAATCAGTTAAAAGTAATGTATTTAATGAATAAGATATTTTTCCTTAACT
AAGACATAAATTAAATTTTAATAAATAGTTATTATTGAGTTGTTAGGAAGTGGTAGAAGT
TTATAAAGAAGTTTTAAGTATCACTCTTCTTTTCTTAATTATAGCTTAGAATTTCTCGTT
TATAAAGTCAACCAATATGCCAAACAAACTCTTATCTCTTTGCTTATAAAGTGGCTTAGG
CTGCGGCACCACACAACACTGCAAGAAGCATTCACAAACAACA
PaGBP1 promoter4
>prP. acutifolius_Phacu.WLD.004G045300
SEQ ID NO: 132
ATTCCTTTCCAGCCTTTGTAGATTCCCTTGTTCTTTTGTTGTCTAATCTAATCATAGTAT
TTTTGTTTGGGTTGCTACGAATTTGAGTCTGAATAGGATTAATACAAGAAACATTACAGA
TTTAGGCTTAAGATTCATTGTCATACCATGAAGAAAATGCAGCTCATTCTATCGTGTACC
TTTGCAACAAAGATAACTTAATTATATAACTTTTTGTGATGACAATTTGTCATCAACATG
TTGCTTTTATATTTTCATTATCTGTGTAATCATGCATATGGAAATGCTAGATAATGTTCT
TATCTTTCGAAACAAAACAATTTGTTTGCTCATAATTTTTCATTACATCAACACACCATT
TTGACTTCTTAACTTAATTGTAGTCTTAAGGTTTTAATTTGAAATGGAGCTTAATATATG
GTGTGATCCATGAAAGCAATTTCAACCAGCTGATAAGACTTCATTGTTCTTACTGCAAAC
TTTTGATTGTTATAAGGGAAACTTCAGTCAAAATTATATATAGGGATCAAAATTCAGTTC
TAGAAATAGAAAACGAAATATGAGTATTGATTTAAAAACTTCTTTTGTACATGAATCACA
TTGAAAATAAGTTATAAATTGAATAAGTCAAACTCAATTTTAATTACACATTTTTAAATA
AATGACTAAGTAGGATTTTTTAATTTTTAACAGTTTTTTTTATCAATTAGTAACTGTTTT
TTACTAATTAAAAACATTTCTAAGTCTCCAAATTAAGTTTAAATCCAATCAACAACAAAT
AAAAAATATAAAAGTTTAAATCTAAACAAACAGGAAGTGTATCGAATATTATATTACATT
TTGAAAATTTATTCTTTATTTACAGTACAACATACATCATAAGTACTAAATAAATAACAC
TTAAAATATATTAAATGTTTTAATTTAAATTGAATTAAATAAATTTGAAGTCTTTTATAT
GTATTTTGAATACTTAATGTTTTTTTTTTAAAATTAGGTATATAATATAACCATTTTAGT
TTAATTTTCTTTTATTACTTATNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNAATA
AAATTTATTTAGAGACTTAAGTTAATATTTTATAATTTTTTTAAGTACTTAATTTATAAA
AAATAATTTTTTGGGTATTTTAATATATATATATATATATATATATATATATATATATAT
ATATATATATACACGTTTTTTCTTTGTCCACCTTCATGGTTTTTCATGAATTCAACACAT
AGTAGGATGTTTCTTCCTACACCTCTATAGCTTCTTGTGGCCCATGTTTTTTTAAAATAC
TAAAAATATCCTTTATTTTACTGATATTTTTTATTTCAAAATAAATTTGAAATATTTTTC
GGATTCTATAAGTTTATTTAAAAATACATTTTAAACTATGCAATTGGAAGATATTTTCGT
ATTATAGGAATTTGGAAAAATTACATAATCCAAAAAGATATTTATGAATTCAAAAATACA
TTTTGGATTCTAAAATTCAATGTAAAATCCAGAATGTATTTTTTAAGAATAAAATAGTTT
TTTTTCTGACCTATGGGATGCTAGAAGAAGTTATGGAGATGCAGGAAAAACAACTTACAA
AAGTAATGAAAAAATCCAAATGGTTTAAGCTTAAATTTTGAGATATTTCAAAAATTTGAA
AAGGTAATTTCGAAATATATTTTTGAATCCATAAATGAATTATGAATTTTTACATTAAGT
TATTCAAAATATATTTCTAAATTTTTAAAAAAAATTATGAATTTATAAGTTACATCATTT
AAAATGCATTTTCATATTCAATAATTTGGAAATGTGTCTAGCTTGTATAATTTAGAATAT
ATTTTCATGTTATACAATTTCTTTTATCATACCATCTAAGATGTATTATGTATTTTTGTA
TTATGCAATATGAAAATATATTTAGATTGTATAATCTAAAATAATTTTTGTGTATACAAT
TTAAAAATATATTTTATATTGTTGGAGATCCCATATCGATTAAAGATGAGAATTTTTTAT
TCTATATAAGTGAGTGGAAATCTCATCCCATGAGCCAATTTTATGAAATTGAGTTAGATT
TAAAGAACTTTGTAATATGGTATCAGAGTCATTTGAAAAATATTCAAGATTATAAAAAAT
TATTATACTTTAGATTATATAACTCAGAATTTTTTTTAGTTATGTAATCTAGAATGATTT
TAATTATACATTATACAATTTAAATAAATTTTTACATTATGTAAAATATAATAGTTTACA
ATCTATACCACATTTGAAATGAAATTTTAGATTAAACAACTTCAAATATTGACCCATAAA
TAATTTGAACTTTTCTCGCAAAAAAAATATGGAATGAAGAAAGAAGTTGCCTTGCATCAA
TAGATTTATAAGACAATAAAGTGTTGCTTGGTGTTAGATATATTTTTTTTAAAAGGATGT
GATTTCATTGTAGAAAGCAATACATAAACTGTATCAATCATCACAGTGTGAAGTGATGGA
ACTCTGTTTTTTAATTTACCTCTAGGTGAAGTAATGCATGGATAATTTAGTTGCATAGGA
AAATAAATCTCTCAAACTCTATTTAGAATTACTGTTCTTAAAAATTGTGAGAAAGTAATA
TGATTCAATTTAGTTTTTTAAAAATTTAGTATTTTGACTCTAAATCATAATTGTATTGTG
AATGATATACAACATAATCTACTACTAAAAAAGTAGCTTCTCCATATAAAAATAAAAAGA
AATTTTAAGGAGAAAACGAACAAGTTAAAGGAAGAAAAATTTTTTCTTAACACTCTAATA
ATAAAAAAAAATCACTCAACAACTATCTTTTAAAATAAAATATTGTTTTATAATAAAATA
ATAAATTTTTTAGATATGAAGTGATCCTTACCCCATTTTCTGCACTCAAGCCAAAGAATG
GAATCATTTCTTCCATTTTTCTGTTTCTATGAACATTCTTCCATTCTCTTCAAACAAACA
ATTCCTTTCTTCATAAATTTCAAAATTCCTTGCTTTTCTTTTCATCTTCATTTTTCTCTT
CTACATTAAGTACAGTAACCAACCCTTTCTATTTCCATATCCAACTAACTTAACGTCATC
CAAACTCACC
PaGBP1 promoter5
>prP. acutifolius_Phacu.WLD.004G045200
SEQ ID NO: 133
TCTATCACTCTCGGCGTGAGGGGGGTGTGATGGAGATCCCACATCGACTAGAGATAAGGA
CATTTCATTGTATATAAGTGGGTGCAAACCTCAACCCTATGAGCCGGTTTTATGGGGTTG
AGTTAGGCTTAAAGTCCACTTTGTAACACATACAATATTTGAATGAATTGGTTTAATAGT
ATATGAGGGTAGAACAACAATTGAAAAGAAAATCTACCTCTAAGAATGATTGTCCCACCA
TTTCCTACCCTAATAAAGAATATAAGAGGGAGAGACCCTTCCAAATCAAGGAATGAAGGG
CCACGAGAAAGGAATAGGAAAAAGGAAAAGAAAGAAAAAACAAAGAGGAAAAAAATTACA
AAACTAAATAATCGTTTAAAGAAACTAAAGCTAGAGATGTCCTATTTTTTAAATATCTTA
TTAGAAGGCACTATGATGTTAAGTGTCCCAATAGAATAACAATGTTTTTAAAATATCAAA
AGATTGAAAGTCAGGATGGAGCTCAATCTTCACCTAGTGAAGATGAAACTCTAGATTCTG
AATAGGAAGCTATACCTTCTGAATGAAAATTGTTAGTGGTAAGAAGACTTCTTAAAAGTA
TCCATAGAACTTGAACAGTTTCAAAGAGAAAACCTATTCCACACCAAATGTAAAGTCTTT
GTAAAAAATTATTATTTGATTATGGATATTGACTCTTGTTGTAATTGTTGTAGTTTTGGT
TGGTAGACAAGCTAGCCTTAATTGTTACATCATGCCCAAAACCTTACAAACTTCAATGGA
TCAAAGATGATGGTGGTGTAGTAGTTAATCAACAAATGAGTATCTCAATTTCTCTAGGAA
ATTATAAAGATAAGTTTAATGTGACATGGTCTCTATGGAAGCTTGACATATTTTACTTGG
TAGACCATGATAATTTGATACAATCTATCCATGATGGTCTTATAAATAAAATAAGTTTCT
CTCATAAGGACAAAAATATAATCGTGTGTCCTCCCACACCTTAACAAGTGAGAGAGGACC
AAATAAAAATGAAAAAAAAAAACTTGAAGAGGAGAAAAGATAGAGAAAAAAACTAAGTCG
GAAAATCTCTCTCACGCTTAAAATAGAGGGTGAGAAGGAGTTGAGTTTGGAGGTTTGTGT
CCCTCCAAAGTAGTTGTTCAACAAAAATCTTTGAAAAATAAAAATAATAAGTCTCTTCTA
GTTGAACAACCTATTTTCCTTTTCTATTGCAAAGAGATACTTGCTACCTCGAATCTTGAA
CTTTGATTCTCTATTACATAGGGTTCAACAACCTTAAAATAATTTCTTAAAATAATTTGG
TATTTTTTTCTCAAGTACGTTCCTCATGGACTTCTATCTTTAAGAGAATATAATATGAAA
TAGACTTAGCCTTGGAGCAGCCTACTGAATAGACCAACTTATACGACTAACCTCAAAGAA
ACTAAAGAGATGGAAAATCAAGTAAGTGACTTGCTAAAAAAGGATTGAGTACAAAAGAGT
CTCAATCCTTGTGTTGTACCAATATTTTTTACCCTTAAGAAGAATGGGTCATGGAAATTG
TAGGATCATTAACAACATCACTATAAAATACACGTACCCAATTCTTAGGTTAGATGACAT
GTTGGACGAGTTACATGAAACTAACATTTTTTAAAATTAATCTTTAAGAGTAATTATCAT
GAAATAAGAATCAAAGAAGGATATGAATAACAAATTGCTTTTAAGATCCAGTTTGGATTG
TACAAATGGTTAATTATGCATCTTGACTTAACCAATACTCCTATTACCTTTATGAGATTA
ATTAATCATGTACTTAGTGATTGCATAAGAATATTTGTGGTTGTCTACTTTAATGACATC
TTAATCTATATAATAATTTAAATAAGAACTTAAATGAATCAGGTTCACTCCATATAAATT
TTGAATTCTTTTACTCAAGTTAGCAATATTTTAGAACTATAATAAATGTGAGTAGAAGTA
ACTTACAACTAATTTCTCTTAATTACTAATCTATTTTATTTCTAACTTGCATCCAATGAA
AATTTAGGCTAGATTTATGTGTTATGAAAATGAAATTTTGAAATAAAAGTAAAATTAAAA
AACTAAAATTTCATTCTGAAACATCAAATCCAAAATACAAGTAATGCATCTCACGATTAT
TTCTTTTTAATAGAAAACAAAATCTAAAAACATATTTTAAAAAACAAAAATATGAAAACA
TATATTGAAAAACCTATTCTGGAAAGTCTAATTCAACAAAATATTCTAAAAACTCAAATC
TAACAAAAGCCAAGAGAAAGTAACATTTAAGGCAATTTAATACATGGACTTTGGGAACAC
AAAGATCAGAAGTTGTTTGTAGCCTATAATTGAAGTCTCTTCCCCCTACATCATAAATCA
TAGTAATTCCTTAACCGCGTAAAGTTCCTTAGATTGTGTCTTTCCTTCACACTAGGACAA
CACATGCCCCATAAATTCTAACTGCATATCATTACGCCATCCCTTCCTTCCACTTTCCTA
TTCACAAACCTTCTCACTTCCTTTCTTCACACAATCTAATTCTCTAACCTTCCAACCCCA
ACAACATACCCTAATCCCTTCACCATTCAACCTTCATAAACCCCCTCTTACCCATCTCAT
TCAATCATCTCCAATCCTATCCCATGCAAAATCATAACCCAATAAAACCATTCTCCTTGT
CAATTTAGTGTCACAATAATCACACTTTGTTGCTTAACCAATTCTCAATACCTCAATTCC
CTAGGATCAAGGTTCACACATATCATACTAAGGCTCTGTTTTTTTGTCTTCTCTAGAGCT
TGTCTTTCTTAAAAAAATATATTTATTTTTTTAATATAGGAATTTGTAGTTTATGATTAC
TTTTTTTTAAAAAAGAATTCTTTTTTAGCCTCATACTTTTGCAAGTAGTCTTCTAATAAT
TAATTTTTCTCTAACACTAAAATAATATTGCAAACATAGATTTAGTCAACAACTCAGAAA
PaGBP1 promoter6
>prP. acutifolius_Phacu.WLD.004G045100
SEQ ID NO: 134
AATGAAAGAAAATACAACTATGTTACAAATAAAGAAATATCAATTTTGTATATACTATAG
TTACAAATAAAACTAAAAAAACGAATGAATAATAAACAAGTTAACTTTTAAAATTGAGAA
TATTACATGTTAGAATTACATTTAGTAAATTCAAATATAACCTAAAATATCTATAAAAAA
AACACTAAACAAATGAACCATAAATCAAATTTTCTATTATAAAGAATAGGAAATCAAACT
CGTAAGAGTTCTTACAAAATTTTAATAATTTTAGTTTTAAGTTTCTTTTCATTACTTATT
TTAAATAATTATAATAATGATAATTGATATGATGTTTTCAATCATCTCTTGTCTAGAAGT
TGAGGATAAAAGAAATTTAAATATATCTTCTTCCCTTAATTTTCTAAGGTGCTTTTGAAG
GATAAAATCGTGATGAAACTCCGAGTTCCAAAATAGACAATACTTTAGGTATAGTTATTG
ACCCTAACAATGTTATCTTAACAAACTTGAGGTCAAAACTCTTCTTTTCCTTAGACTTCG
ATGTGAGGCTTGTTCTGAAATAATCGATAGTCTTATATTACTTAAAAGTCAAGATTATGA
ATAATTTAAATATATCTTCATCCCTTAACATTTTGAAATGTTTTTTAAGAGAAAAAAATC
ATGATGAAATTTCAAGTTCTAAAGTGCATTATATCACAAATAAATGTGATTATTGACTCT
AATAATGTTATTTCAAAAAAAAAAATTATACAACTATAACGAGTCAAACTCCTTTCTAAT
AACAACAATAATGTATTATTTGTGCTTTCATCTATGGTTAGTTCTACCTTATTAGTTTTA
TGTTAAGGCTTATAATGTGAAATAATTCATTCGTTTTTCTACGAAACTATTATTTTCTTA
GAAAGAGTTTCAAAAAAGTGTTTAAATAATAATAATAATANNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNGCAACTA
ATCAATTATCTTATAAAATAATCAATTGGTATAAGTAAAATAATTCATTATTTGAAGTTA
GAAAAAAATACAATATTTGTGGAGAATCTATTAAGATTAAAAGTAATAAATTCTATCATT
TTTATTAAATTTGTATTTTTTTATTAAAATTAATAAATAAAACTAATCAATTTACAAATC
ATTCTTGCTTATTTTTAATTGTTCTTCAATAACTCTACAAGATTAAACATAATTTTTGCT
CAAAAATAGTAAATGATATATCTCTTTATTTTTTTGGATAATCTATTACATCAAATGATA
TATTTCCATATTTGAATTTTTCAGTCATATTTTTGGTGTTTCGTGTAATAGGCATTTACA
TGTTGATCTAAAAATTAAAATACTTTAAATGTGGGATAGTTATAGCCAAATATTCTACTT
GGTGTGTTATTTTACATTAATTAATACTTATAACTTTTTAATTTAAACATTTCTTTTTTA
AATATATGTTACTTATCTTCTTTACTTATTCATTGAAAATAATTATACATATGTTTATCT
CATTATATTAAATATTTTTATTTCTATTTAAAATTCTAGATATATTAAAAAATTACTCTT
AAATATTAAATTTATACCCTAATTTGAAGGGTGATTAGAATTCTCCATTATCATAATAGT
TGTTTGTGTGGAAAGTATGTAGTGGAGAGCACATCCTTCCTCCATGCTTCTATTTTTGGG
TGTTCACTTCTTCCTCTCTTTCATTCCAAAAAAGAATAAGACACTTGAAATAGAAAAAAA
AAATAATTAATTTTACAGACATAAATTAAAAATTATTTACGTTTTCTCTAATTTTAGAAA
TTAATTTTACAACAAAATACAATGTTGTGTATATGTTTACAAACGTGAGGTATATATCTT
GGTAGTTAAAACTTTATAATTAATTAGATACTAATTTAAAAATTATTTAACAATAATATA
AACTAATTTAGAAATTAAAATTTTTTTAGTTTCTAAAATGATCTCTAATTTAGTTAATAT
AACAATTAATTTTTTTTCTCTAAAATTAATTTTTATTTTATAATTTTTTTTTAGTGTATA
CTTAATTTTGTATCTCGTACAATTCAACTTAATTATATTTTTATGGGTTGGATTGAATTG
TTGTGTAAAATTGGATCCCCTAACATGTTTATAAATTTTGAATATGAAAAACTCAACTTC
ATAATAAATATATAATAAAATATACTAGTATTATTTGTGTGTATATATAGAATAAGAATT
ACCGCTATTTTTAATTATTAAAAATATTTCTTTTGGAAATCGTTTTATTAAAAAAGAATT
ATTTTTTAAATTACTCAATTCATTAAAAATAGTTAGATCATACTAAATAAACAAGTAAAC
AAATTGGATGGGATGCAATTAAATTTAATTAAATAACAAATAAATTAAACTCCACCTTAA
CGTGTTGAGTAGTATATATCATAGACGAGAACGAAAATTCATACATGATAAGACTTTTCC
AAACAATAATATCTTTTGGCTTGCTATCAAAACTTCCACATTCAATATGATTCATATGTG
TATTGATCCATAACTTGTTGTTAAAGATGTTACGTTTTATAAGTTGAGTTAGGCTCTTCC
CTAACATGAGTTTATCTTGTTCCAATTCTTCATTGGTGTGCACATAAATCTGCTTTTATG
TGGGGTACTGGTTTTTTATTTCTGCATCTGAATTTGGTATGTTCCCATTGAGAAACTTCT
GTTTTGCTTCTTGACATGGTCACATTCACTCACCTTTCTTCCCAAGTTTCACTTCCTTTC
AATCAATCATTCAATCCTCTTTACTCACACCCTTCCACGGTTCATTCATCAGAAATTTAG
AATCATTCATACTCTCTAACATCATCATCATCAACACACCAAAACAAAACTTTAACCTCA
ACTTGTACTAAAAGCCAGTTCACCAAATCAATAAACTATGGTCAACTATCCAGCAAGTAT
CCTCCAAAAATCCAAAATCCAAACACCTCTTTCCTACTCATAACAATTTAATAATTAACC
AGTTGTTGATAGAGTAGCCGCCAATAATGAAAAAACACCAAGTCTAAGTAACAACTGCAT
TGACCTCATCCTCTCCAAAACCCTTATATACTCACACCATTCAAGCATTCATCATCATCA
TCACCACAACCAAACACCACTTCCACCACCTTCAGAAGCAGCA
CaGBP1 promoter1
>prC. arietinum_NC_021161.1
SEQ ID NO: 135
TATGTGTTTTACTTTATTTTTATCGAACCAATATCATTTAAGTCATTATCTCGATCATAA
AAATACATATATGATCATTAGTCTACTGCCAAAAGACGATATAGTAAGTCTTACATTTAC
AAAATCATTAGTCCACCTTTGGGAAAAGTGTGCAAAGAACAATTACTAAGTTTTTTTTTT
ATGCTAAATAATACGTACAGATCCTTAACTTAATTTCAGTTAACGTTTTAGTCTTTTATC
TTTTTTTTTTCTTCTCGGTTTGGTTCTTTATTTTAATTTTAAATGACAATTTGATCTTTT
ATGTTTTAAAAATGTAAACAATGTAATCATTTTTTACAAAAATTCATCAAAATTTTCAAA
CAAAACTCATAAAATTAATTATCATCTTCAATATAATGCAAATTTCATCAAACTCATAAC
TTAAATCTTTATATAAACTCATATTTTTATTCATTATTTGATGAATTTGATGTCGTTGGA
GATAAAAATATGAGTTGATTTGAATATTTGAGTTATGCATTTGATAAAATTTGCATTATA
TTGAAGATGATAATTAATTTTATGGGTTTTGTTTGAAAATTTTGATGACTTTTTTGAATT
TTTATAAAATAATAGATAACATTGTTGATATTTTAGAACATAAAAGATTAAATTATCACT
TAAATTTAAAATAAATAATTAAATCAAGAAAGAAAAAAAGATAAACGACTAAAAGATTAA
ATGAAATTAAATTAAGGGATTGTGTGTACTATTTAATTTTTTATAAAAAAAAATATTTTA
AAAAAATAAATTAACTCATGTATACAGATTGTAATCAATACTTTTTTATCCAAACTTGTT
AATTTTTATTTTATTTTTATGCCCTTTGATAAGGATCGATGCTCTGGTGGCTTCCTGGCA
TAGAGGTTCTTTTGCAATAAAATAAAATAATCAATCTCTCGAAATAAGAAATTAGCTAAT
AAGTTAAAAAATAAATAAAACTATGCAAACAATTGTTTATGATTAACACGCTCCGTTGCT
AACGGGTATGTTCAAAAACGTTGCCTAAACACCTTTAAACAATAATATAAAATGCTTTTA
GCATATTAAATTCATTAAAAATATTCAATTGAGTAATTTATGAGGGAGACTCTGATTAAT
ATTTTGATATACTTTTTCAAATATAACGGATGTAAAATGTATATTTTTCTAAAAAGGAAA
ACGGATATTGATTGTACATTTTATTCAAAGATTTTCAGAATATTTGTTTTCAAGTCAACC
TTTACATGTTCAACGATCTTAGCTAAACTCATATTCATTGATATATAAAACCAATCAATT
TCTTTAATAAATAAGAAAAAACAATCGTTTCACCTGAATGTGAAAAATTATAAACTAAAA
AAATGTCAAAATTTAAATGATTCAATATTTTTTCATTTTAAATATATAAATTAGTGAACT
TATATAGTGGAATACGTATAAAAATGATTGTATTTTTTTATCTTTATAATAAAATTAGAA
TTTTAAAAAATAAAATTATGTCTTTTTCAATTTTCCTATTCTTATTATTTTTCGATCCCT
CAAATTTATGAAAAGGAAAAAAACCATATTTCATAAAAAATTCATTTTTACTAACAATTA
TTAAATATTTTATTATTTTATTAAAATTACTAGTAATTTTAATTTAACTATTGTAATTGT
TACAAAATAAATTTTTATTGAAAATAATCTGATTTTTTCTATTACCAAATAAAAATATTT
AACAATTACTAAAATAATCCCTCAAGTTAAATATTTTACTAAATAAAAATTACTCAGATT
ATTTTTCAAAAAATCTTTTCTCGTTAGAATCTTTTCAAATAAAGATATTTTTTACTCAGA
TTATTTTCAAAAAATATTTTTTGACTCAAAAGTTATAAATCTTTTTCAAAAAAAAATTAC
TCAGATTATTTTCCAAATAAATAAATTATTACAATTTTTTTGTATTTACAAAATTTAGTA
AAGCATTTAATTGAGTAATTTGTGAGGAAGACTCTAATTAATATTCCGATGAAAAATATT
AGCAAAATACTATTAAACACATTTTCTAATCTATCATCTTTTATTAGTTAAAATTTACAT
GAGTCTCATAAAATGTAAATGAAATCCATTCAATTTGGTGGGATCTATGTAAATTTCAAT
TAATAAAAAAAATATATTGGAATATATGTATAAGAGAATGTATAGTCAACACTCTTCTAT
TCCCATATACTTTTTTAAACATAACTGATATAGATTGTATATTTTTATAAATATAGTAAT
GATGCGACATCTTTTAAATGACATGATATATAATATGATGATATTGATGGAAGGAATAAT
ATCTATGAATATACAATAAAAACATTCTAAAAATTTTAGTTTTAATTTCTAAAATTATTT
ATTTTTATGATTTAATTAATAATATAAATAGTTAATGTATCTTAAATGGTTTTATATCCT
TAAATTGTATGTTATATCACCTAAAATATATTAAATCTCCATTTTTAATTTAAAAAGTCA
AAAACTATATTAAAATTAATAAATTTTAAAATAGAAGAACAAATTCAACGAATTAATAAA
AGTTGACTATAATTCTTAAAAAAAAAAAAATCAATGTAGATTGCATATTGTCTCAAAAAA
TTTAAAAATATTTGTTTTAAAGTCAACGTTTAAATGTTAAACAATCTTACCTAATTAATT
CATATTAAAAAACTAACTTAAATTAGAATTTTAATTTCAAATTAGAATTAATTTTAAAAA
AAAAAGGTAAAAAAGAAAGATATCAAAAATCAATTTCAAATCTGTAAAATTGATTTTGGG
GTGCTTCAAACAAAAAGCAAACAAGCACATTGTAAAAAGTCTCATCAACCAAACACAAAC
CAAACACAAATTAATTGATATATAAAAAGAGAGGAAAGGAAAAACAAGCACCACACACAA
CACAACTATTGCAACAACTCTCATCAACCAAAAACAAAAAACACAAACATTGTAACA
CaGBP1 promoter2
>prC. arietinum_NW_004515975.1_1
SEQ ID NO: 136
TGGAAATTCAATGTTCATGGTTTGCTCATTATTATTTCCAATGTTAAATGGTTTCTATAT
ATTTAAAAAGAAAATAAGAAACAACTGATTCAATTGCTTAATGAATTTCTGTTAAGAATA
AATCTTTCTATAGAACACAAATTTAAATTTTAATTGAAATAATTATTGATTAAATTTTAT
TTATCTCTTAAATGAATTCTGAATTACTAAAGTTTCATTACAATGGAAACTAGAGGTTAA
AAAAAAAAAAAAAAAAGTAATAAATTTTTAATAACTCTTGCATTGTATTGTAGATTTTTA
ATAAAATCTATTGTGTAAACTTGTAAATTTATTTTATGTTGCTGTACCTATTGTTGATAG
AAAATCCAAAAAGTAAAATTCCATTTACAAACTTGGTTGTTCTAACATCAGCGACTTCAA
TCCAATTACTTATCATACAACAACAAGACAAAGCAAACAATGACAAAGGCCTCAAAAAAA
AAAATACTTAATTGAAAGAATTCAAAAAAAAAAAAAATTAATGAGTAATCAGAGTTTTGT
GTATTGATAAGTAATTAGTGATAGATTGAAGTTGAAATTACAAAATGTACGGAATAATTG
TACCTAATTGGATAAATAAGTGAAAAAGTTTGTGGTATATATTAGTAAAACAAAATAGAA
TAAAAGTAGAAGATAGAGAAAAATAAAAGATACAAAAAATAGTATGCTTTAGAAGATACC
ATATTTTATTTTAATATTATAATAATAGATAATTAATTTGAACAATAATTCATCTCAAGT
TTATAATGAGTTATTAAACATAGAGTAAAATTTGATATGTGTGTGTTATATGCTCTCTTC
AAATTGAATGCTTGCACTTGCACTATGGTGATGGTGAGCATGAAACATTTTTAATACTTT
TTATAGTTGGCACAATGGTGTGCTAATTATATTCAAACCTATTGATATATTCTTATATAT
ACTTTATCCAATTTAATATAATCTATTTTCAATTCAACAATTATGTGTTGTCATGTGTCA
CATTTTCTTTTTTCTTAATTCAGCATAATACTAAGTATATACAATAATTAATTTCTCCAT
GGAATACCAGAAAAGCAAGTACATTATTCTAAGGTTCCTTTCTAACAAAATCATGGTTTT
TACTTGTTTTGTAACTTTCATGTATAAGGACTTATGTTGATCGTGTAATTTGGTCACATT
CATGTACCTTACCTTAAAATTAAATCAATCATTCAATTCTCTTTACTTCCACTCTTTACA
CACCTTGTAATCTCATTTAAGCCGCCATAAAAAACAAACCTTCCCATCAACTTGTGCTAT
AATATTAGTTTACAATTTCAATATTATATAGTCAACTTGACAACCATATTTAATCATGCA
TACATCAACCAAATTACTCAAATCCCATTATTCTCCTATTCATGACAATTTTATAATATA
TGAAGCAACTTGTAGATATATAGAGTAGTTGCCAATATTGATAATAATATATCCGGTGTA
TATATTATCGATATTTCATATTAATGACATATTTAATGTAAGATATGTCTATTTGGTATC
TGACACCAACACTATAATACTGTTTCATTTTTTTAAATTATTACTAATGTCTATATTCAG
TACCGTGTCTGATGTCGAAGTATGTGTACTAAAGAAAAGAAAGAACTCCACTCAGCTCAA
CACATACCCTCAAAGAAACACTATAAAACTCATGATTCAATATCACTACAATATCCATTA
AGACCTTAATCATCATAATCAAACACA
AC
GPB1gF3 Primer
SEQ ID NO: 137
TAAGGAGAATAAGTAAGTAGCCCTTATCA
GBP1gR2 Primer
SEQ ID NO: 138
AGAAGGAGCCCACCAAAGTT
tnt1-R Primer
SEQ ID NO: 139
CAGTGAACGAGCAGAACCTG
tnt1-F Primer
SEQ ID NO: 140
ACAGTGCTACCTCCTCTGGA
GBP1qF Primer
SEQ ID NO: 141
AAATCAATATGTTTGGGTCATGC
GBP1qR Primer
SEQ ID NO: 142
TTGTCGGCCACATATCCTTG
GBP1cIF Primer
SEQ ID NO: 143
ATGTCTTCATCATCTTCTCTTCCTTT
GBP1cIR Primer
SEQ ID NO: 144
TCATCTGCTATGGATCCACC

1. A genetically altered plant wherein expression of a GBP1 nucleic acid sequence or function of the encoded GBP1 protein is reduced or abolished in said plant.

2. The genetically altered plant of claim 1 wherein said plant comprises a mutation in the GBP1 nucleic acid sequence encoding the GBP1 protein or in a promoter nucleic acid sequence that regulates expression of GBP1.

3. The genetically altered plant of claim 1 wherein said GBP1 nucleic acid sequence is selected from SEQ ID NOs: 1 to 48 or a homologue, paralogue, orthologue, or functional variant thereof with 70%, 80%, 90% or 95% sequence identity to any one of SEQ ID NOs: 1 to 48.

4. The genetically altered plant according to a claim 1 wherein said mutation:
a) comprises the deletion, insertion, replacement or addition of one or more nucleic acids in the nucleic acid sequence;
b) comprises the insertion of a Tnt-transposon into the nucleic acid sequence;
c) is introduced using targeted genome modification;
d) is introduced using a rare-cutting endonuclease, for example a TALEN, ZEN or CRISPR/Cas9;
e) modifies symbiosis with a rhizobacterium in root nodules of the plant; and/or
f) modifies symbiosis with a rhizobacterium which increases the nitrogen fixing in root nodules of the plant.

5-9. (canceled)

10. The genetically altered plant of claim 1 wherein the plant is heterozygous or homozygous for the mutation.

11. The genetically altered plant of claim 1 wherein the expression of the GBP1 nucleic acid sequence is reduced or abolished in said plant using RNAi silencing.

12. The genetically altered plant of claim 1, wherein the plant is a legume plant or a non-legume plant.

13. The genetically altered legume plant of claim 12 wherein:
a) said legume plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum); or
b) said non-legume plant is selected from Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

14-15. (canceled)

16. The genetically altered non-legume plant of claim 13 wherein the plant is selected from Cassava (Manihot esculenta), Rice (Oryza sativa) or Sorghum (Sorghum bicolor).

17. A method for modulating nitrogen fixing symbiosis in a plant and/or increasing plant biomass, the method comprising reducing or abolishing the expression of a GBP1 nucleic acid sequence encoding a GBP1 protein and/or reducing or abolishing the function of the GBP1 protein or a homologue, paralogue, orthologue, or functional variant thereof.

18. The method of claim 17 wherein the method comprises introducing a mutation in the GBP1 nucleic acid sequence encoding the GBP1 protein or in a promoter nucleic acid sequence that regulates expression of GBP1.

19. The method of claim 17 wherein said GBP1 nucleic acid sequence selected from SEQ ID NOs: 1 to 48 or a homologue, paralogue, orthologue, or functional variant with at least 70%, 80%, 90% or 95% sequence identity to any one of SEQ ID NOs: 1 to 48.

20. The method of any of claim 17 wherein said mutation comprises:
a) the deletion, insertion, replacement and/or addition of one or more nucleic acids into the nucleic acid sequence; and/or
b) the insertion of a Tnt-transposon into the nucleic acid sequence selected from SEQ ID NOs: 1 to 48.

21. (canceled)

22. The method of any of claim 17 wherein the method comprises:
a) introducing said mutation using targeted genome modification;
b) introducing said mutation using a rare-cutting endonuclease, for example a TALEN, ZEN or CRISPR/Cas9;
c) introducing a heterozygous or homozygous mutation into the plant;
d) applying a mutagenic composition to the plant; and/or
e) introducing into said plant a dsRNA molecule suitable for RNAi silencing.

23-26. (canceled)

27. The method of any of claim 17 wherein the plant is a legume plant or a non-legume plant.

28. The method of claim 27 wherein:
a) said legume plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum); or
b) said non-legume plant is selected from Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

29-30. (canceled)

31. The method of claim 28 wherein the plant is selected from Cassava (Manihot esculenta), Rice (Oryza sativa) or Sorghum (Sorghum bicolor).

32. An isolated mutant GBP1 nucleic acid sequence encoding a mutant GBP1 protein wherein expression of the GBP1 nucleic acid sequence or function of the encoded GBP1 protein is reduced or abolished in a plant.

33. The isolated mutant GBP1 nucleic acid sequence of claim 32 wherein the mutant GBP1 nucleic acid:
a) comprises a mutation in the GBP1 nucleic acid sequence selected from SEQ ID NOs: 1 to 48 or a homologue, paralogue, orthologue, or functional variant with at least 70%, 80%, 90% or 95% sequence identity thereto; and/or
b) comprises a deletion, insertion, addition and/or replacement of one or more nucleic acids and/or a Tnt-transposon inserted into the nucleic acid sequence selected from SEQ ID NOs: 1 to 48.

34. (canceled)

35. The isolated mutant GBP1 nucleic acid sequence of claim 32 wherein the plant is a legume plant or a non-legume plant.

36. The isolated mutant GBP1 nucleic acid sequence of claim 35 wherein:
a) said legume plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum); or
b) is selected from Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

37-38. (canceled)

39. A vector comprising an isolated nucleic acid of any of claim 32.

40. A host cell comprising a vector of claim 39.

41. A method for producing a plant with modulated nitrogen fixing symbiosis, comprising introducing a mutation into a GBP1 nucleic acid or in a promoter nucleic acid sequence that regulates expression of GBP1.

42. The method of claim 41, wherein:
a) said method comprises introducing a mutation in the GBP1 nucleic acid sequence selected from SEQ ID NOs: 1 to 48 or a homologue, paralogue, orthologue, or functional variant with at least 70%, 80%, 90% or 95% sequence identity thereto;
b) said mutation comprises the deletion, insertion, replacement and/or addition of one or more nucleic acids into the nucleic acid sequence and/or insertion of a Tnt-transposon into the nucleic acid sequence selected from SEQ ID NOs: 1 to 48; and/or
c) said method comprises introducing the mutation using targeted genome modification; and/or
d) said method comprises introducing the mutation using a rare-cutting endonuclease, for example a TALEN, ZFN or CRISPR/Cas9.

43-45. (canceled)

46. The method of any of claim 41 wherein the plant is a legume plant or a non-legume plant.

47. The method of claim 46 wherein:
a) said legume plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum); or
b) said non-legume plant is selected from Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

48-49. (canceled)

50. The method of claim 47 wherein the plant is selected from Cassava (Manihot esculenta), Rice (Oryza sativa) or Sorghum (Sorghum bicolor).

51. A method for identifying a plant with altered nitrogen fixing symbiosis compared to a control plant, the method comprising detecting in a population of plants one or more polymorphisms in a GBP1 nucleic acid sequence.

52. The method of claim 51 wherein the GBP1 nucleic acid sequence is selected from SEQ ID NOs: 1 to 48 or a homologue, paralogue, orthologue, or functional variant with about at least 70%, 80%, 90% or 95% sequence identity thereto wherein the control plant comprises a GBP1 nucleic acid that encodes a protein having a wild type GBP1 protein.

53. The method of any of claim 51 wherein the plant is a legume plant or a non-legume plant.

54. The method of claim 53 wherein:
a) said legume plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum); or
b) said non-legume plant is selected from Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

55-56. (canceled)

57. The method of claim 53 wherein the plant is selected from Cassava (Manihot esculenta), Rice (Oryza sativa) or Sorghum (Sorghum bicolor).

58. A detection kit for determining the presence or absence of a polymorphism in a GBP1 nucleic acid sequence in a plant.

59. The detection kit of claim 58 wherein the plant is a legume plant or a non-legume plant.

60. The detection kit of claim 59 wherein:
a) said legume plant is selected from barrel medic (Medicago truncatula), alfalfa (Medicago sativa), pea (Pisum sativum), broad bean (Vicia faba), red clover (Trifolium pratense), white clover (Trifolium repens), subterranean clover (Trifolium subterraneum), birds treefoil (Lotus japonicus), blue lupin (Lupinus angustifolius), white lupin (Lupinus albus) Cowpea (Vigna unguiculata), Common Bean (Phaseolus vulgaris), Soybean (Glycine max), pigeon pea (Cajanus cajan), lima bean (Phaseolus lunatus), tepary bean (Phaseolus acutifolius), and chickpea (Cicer arinetum); or
b) said non-legume plant is selected from Tomato (Solanum lycopersicum), Potato (Solanum tuberosum), Pepper (Capsicum annuum), Tobacco (Nicotiana tabacum), Grapevine (Vitis vinifera), Cucumber (Cucumis sativus), Citrus (Citrus spp.), Apple (Malus domestica), Strawberry (Fragaria x ananassa), Wheat (Triticum spp.), Cassava (Manihot esculenta), Thale cress (Arabidopsis thaliana), Rice (Oryza sativa), Sorghum (Sorghum bicolor), Pecan trees (Carya illinoinensis), Barley (Hordeum vulgare) or Oats (Avena sativa).

61-62. (canceled)

63. The detection kit of claim 60 wherein the plant is selected from Cassava (Manihot esculenta), Rice (Oryza sativa) or Sorghum (Sorghum bicolor).


SEQUENCE LISTING (144 sequences)

SEQ ID NO: 1 - DNA, 1992 bp, Medicago truncatula
  source 1..1992: mol_type = genomic DNA
atgtcttcatcatcttctcttcctttcctatttcctcaaactcattcaacagtcctcccaaacccttcaaacttcttctcacaaaacctactatccacacccctccctacaaactctttcttccaaaactttgttctccacaatggtgacacacctgaatacattcacccttacctcatcaaatcctcaaacttttccctctctatttcctaccctcttctcctcttttcagcaacaatgttgtaccaagttttttcaccagatctcacaatttcatcctcacaaaaatctcacacaaacacaacaaaaaaccatgttatctcatcatatagtgatcttggtgtgactcttgacattccctcttcaaatctaagattctttttagttagaggaagcccttttataactgcttcagttacaaaaccaacctcactttcaatcacaacacttcataacatagtttctttgtcttctttcgacgacaaaaacaccaaacatacccttcaacttaataacactcagaaatggatcatatacacttcttcaccaataaaattcaaccatgatggttctgagattgtatccaatccattttccggtataatccgtatcatagtcattcctaataccaaatttgagaaaattcttgataaattcagctcttgttaccctgtctctggtgatgcaaacatcaagaataaatttcatttggagtataaatggcaaaagaaatgttctggtgatttactcatgctagctcaccctcttcatgttaagcttctatcacaaagtaataatgttaatgttactgttttgcatgatttgaagtatacaagtgtcgatggtgatctcgttggtgttatcggagattcatggatattggaaactgatcctgttaatgtaacatggtattctagtaaaggtgttacaaaagaatcacatgatgagattgtttcggctcttgttaaagatgtgaaggagctgaattcttcagcaataacaacaaatggatcttatttttatggtaagattgtttcaagagctgcaaggtttgcattgatagctgaagaagtatcttaccctaaagtgattccaattatcaagaattttttgaaggaaactattgagccatggttgaatggaactttcaaagggaatggtttcttgtatgaaaaaaaatggggtggattagttactaaacaaggggttaataattcagttgttgattttggttttggaatttataatgatcatcattatcatttaggttattttctttatggaattgctgttcttgcaaagattgatccattttggggacaaaagtataaaccacaagcttattcacttttgcaagattttatgaacttgggccaaagggataacaaaaactatccaactttaaggtgttttgattttttcaagttgcattcttgggctgcaggagtgactgaatatgaaaatggaaggaatcaagaaagttcaagtgaagcagtgaatgcatattattcagcagcattgataggtctagcatatggcgacaaagatcttgtcgccattggatcaacgcttttagcgttggaaatcaatgctacacaaacttggtggcatgtgaaagttgaaaataatttgtatggagaagagtttgcaaaagaaaataggattgttggtattttgtgggctaataagagagatagtaaactttggtgggctccttctgaatgtagagggtgtagggttagtatccaagttatgcctttgttgcctattactgagtctttgtttaatgatggtgtttatgctaaggagctagtggaatggacactcccttctttgaagaatgacacaaatgatgatagatggaaagggtttatctattctttgcaaggaatttatgataaagaaaatgcattgaagaagattagaatgttggaaggttttgctaatggaaactcattcagtaatctcttatggtggatccatagcagatga

SEQ ID NO: 2 - DNA, 1992 bp, Medicago sativa
  source 1..1992: mol_type = genomic DNA
atgtcttcatcatcttctcttcctttcctatttcctcaaactcattcaactgtcctcccaaacccttcaaacttcttctcacaaaacctactatccacacccctccctacaaactctttcttccaaaactttgttctctacaatggtgaaacacctgaatacattcacccttacctcatcaaatcctcaaacttttccctatctgtttcataccctcttctcctcttttcaacagcaatgttgtaccaagttttttcaccggatctcacaatttcatcctcacaaaaaactcacacaaatataccaaaaaaccatgttatctcatcatatagtgatcttggtgtgactcttgacattccctcttcaaacctaagattctttttggttagaggaagcccttttataactgcttcagttacaaaaccaacacctctttcaatcacaacaattcatagtataatttctttgtctccttttgataagaaaaaaaccaaatacacccttcaactcaataacaatcagaaatggatcatatacacttcttcaccaatcaagttcaaccatgatggttcagaggttatgtccaatccattttccggtataattcgtattgtcattgttcctaattccaaatatgagcaagttcttgataaattcagcacttgttaccctgtctctggtgatgcaaacatcaagaataaatttcatttggagtataaatggcaaaagaaatgttctggtgatttactcatgctagctcaccctcttcatgttaagcttctatcacaaagtaatgatgctagtgttactgttttgcatgatttgaagtatacaagtattgatggtgatctcgttggtgttatcggagattcatggatattggaaactaatcctgttaatgtaacatggtattcaagtaaaggtgttacaaaagaatcacatgatgagattgtttcagctcttgttaaagatgtgaaggagctgaattcttcagcaataacaacaaatggatcttatttttatggtaagattgtttcaagagctgcaaggtttgctttgatagctgaagaagtgtcttaccctaaagtgattccaattatcaagaattttttgaaggaaactattgagccatggttgaatggaactttcaaagggaatggttttctctatgaaaaaaagtggggtggattagttactcaacaaggtgttaatgattcaggtgttgattttggttttggaatttataatgatcatcattatcatttagggtattttctttatggaattgcagttcttgcaaaaattgatcctttttggggacaaaagtataaacctcaaacttatgcacttgtgaaagattttatgaacttgggccaaagggataacaaaaactatccaactttaaggtgttttgatttcttcaagttgcattcttgggctgcaggagtgactgaatatgaaaatggaaggaatcaagaaagttcaagtgaagctgtgaatgcatattattcagcagcattgataggtctagcatatggtgacaaagatcttgttgatattggatcaacacttttagcatttgaaatcaatgctacacaaacttggtggcatgtgaaagttgaaaaaaatttgtatggagaagagtttgcaaaagaaaataggattgttggtattttgtgggctaataagagagatagtaaactttggtgggctccttctgaatgtagagggtgtagggttagtatccaagttatgcctttgttgcctataactgagtctttgtttaatgatggtgtttatgctaaggagcttgtggaatggacactaccttctttgaagaatgaaacaaatgatgatagatggaaagggtttatctatgctttgcaaggaatttatgataaagaaaatgcattgaagaagattagaatgttggaaagctttgctaatggaaactcattcagtaatctcttatggtggatccatagcagataa

SEQ ID NO: 3 - DNA, 1992 bp, Medicago sativa
  source 1..1992: mol_type = genomic DNA
atgtcttcatcatcttctcttcctttcctatttcctcaaactcattcaactgtcctcccaaacccttcaaacttcttctcacaaaacctactatccacacccctccctacaaactctttcttccaaaactttgttctccacaatggtgaaacacctgaatacattcacccttacctcatcaaatcctcaaacttttccctatctgtttcataccctcttctcctcttttcaacagcaatgttgtaccaagttttttcaccggatctcacaatttcatcctcacaaaaaactcacacaaatataccaaaaaaccatgttatctcatcatatagtgatcttggtgtgactcttgacattccctcttcaaacctaagattctttttggttagaggaagcccttttataactgcttcagttacaaaaccaacacctctttcaatcacaacaattcatagtataatttctttgtctccttttgataagaaaaaaaccaaatacacccttcaactcaataacaatcagaaatggatcatatacacttcttcaccaatcaagttcaaccatgatggttcagaggttatgtccaatccattttccggtataattcgtattgtcattgttcctaattccaaatatgagcaagttcttgataaattcagcacttgttaccctgtctctggtgatgcaaacatcaagaataaatttcatttggagtataaatggcaaaagaaatgttctggtgatttactcatgctagctcaccctcttcatgttaagcttctatcacaaagtaatgatgctagtgttactgttttgcatgatttgaagtatacaagtattgatggtgatctcgttggtgttattggagattcatggatattggaaactaatcctgttaatgtaacatggtattcaagtaaaggtgttacaaaagaatcacatgatgagattgtttcagctcttgttaaagatgtgaaggagctgaattcttcagcaataacaacaaatggatcttatttttatggtaagattgtttcaagagctgcaaggtttgctttgatagctgaagaagtgtcttaccctaaagtgattccaattatcaagaattttttgaaggaaactattgagccatggttgaatggaactttcaaagggaatggttttctctatgaaaaaaagtggggtggattagttactcaacaaggtgttaatgattcaggtgttgattttggttttggaatttataatgatcatcattatcatttagggtattttctttatggaattgcagttcttgcaaaaattgatcctttttggggacaaaagtataaacctcaaacttatgcacttgtgaaagattttatgaacttgggccaaagggataacaaaaactatccaactttaaggtgttttgatttcttcaagttgcattcttgggctgcaggagtgactgaatatgaaaatggaaggaatcaagaaagttcaagtgaagctgtgaatgcatattattcagcagcattgataggtctagcatatggtgacaaagatcttgttgatattggatcaacacttttagcatttgaaatcaatgctacacaaacttggtggcatgtgaaagttgaaaaaagtttgtatggagaagattttgcaaaagaaaataggattgttggtattttgtgggctaataagagagatagtagactttggtgggctccttctgaatgtagagggtgtaggcttagtatacaagttatgcctttgttgcctattactgagtctttgtttaatgatggtgtttatgctaaggagttagtggaatggacactaccttctttgaagaatgaaacaaatgatgatagatggaaagggtttatctatgctttgcaaggaatttatgataaagaaaatgcattgaagaagattagaatgttggaaggttttgctaatggaaactcattgagtaatctcttatggtggatccatagcagataa

SEQ ID NO: 4 - DNA, 1992 bp, Medicago sativa
  source 1..1992: mol_type = genomic DNA
atgtcttcatcatcttctcttcctttcctatttcctcaaactcattcaactgtcctcccaaacccttcaaacttcttctcacaaaacctactatccacacccctccctacaaactctttcttccaaaattttgttctccacaatggtgaaacacctgaatacattcacccttacctcatcaaatcctcaaacttttccctatctgtttcataccctcttctcctcttttcaacagcaatgttgtaccaagttttttcaccggatctcacaatttcatcctcacaaaaaactcacacaaatataccaaaaaaccatgttatctcatcatatagtgatcttggtgtgactcttgacattccctcttcaaacctaagattctttttggttagaggaagcccttttataactgcttcagttacaaaaccaacacctctttcaatcacaacaattcatagtataatttctttgtctccttttgataagaaaaaaaccaaatacacccttcaactcaataacaatcagaaatggatcatatacacttcttcaccaatcaagttcaaccatgatggttcagaggttatgtccaatccattttccggtataattcgtattgtcattgttcctaattccaaatatgagcaagttcttgataaattcagcacttgttaccctgtctctggtgatgcaaacatcaagaataaatttcatttggagtataaatggcaaaagaaatgttctggtgatttactcatgctagctcaccctcttcatgttaagcttctatcacaaagtaatgatgctagtgttactgttttgcatgatttgaagtatacaagtattgatggtgatctcgttggtgttatcggagattcatggatattggaaactaatcctgttaatgtaacatggtattcaagtaaaggtgttacaaaagaatcacatgatgagattgtttcagctcttgttaaagatgtgaaggagctgaattcttcagcaataacaacaaatggatcttatttttatggtaagattgtttcaagagctgcaaggtttgctttgatagctgaagaagtgtcttaccctaaagtgattccaattatcaagaattttttgaaggaaactattgagccatggttgaatggaactttcaaagggaatggttttctctatgaaaaaaagtggggtggtttagttactcaacaaggtgttaatgattcaggtgttgattttggttttggaatttataatgatcatcattatcatttagggtattttctttatggaattgcagttcttgcaaaaattgatcctttttggggacaaaagtataaacctcaaacttatgcacttgtgaaagattttatgaacttgggccaaagggataacaaaaactatccaactttaaggtgttttgatttcttcaagttgcattcttgggctgcaggagtgactgaatatgaaaatggaaggaatcaagaaagttcaagtgaagctgtgaatgcatattattcagcagcattgataggtctagcatatggcgacaaagatcttgtcgccattggatcaacacttttagcatttgaaatcaatgctacacaaacttggtggcatgtgaaagttgaaaaatatttgtatggagaagagtttgcaaaagaaaataggattgttggtattttgtgggctaataagagagataataatctttggtgggctccttctgaatgtagagggtgtaggcttagtatacaagttatgcctttgttgcctattactgagtctttgtttaatgatggtgtttatgctaaggagctagtggaatggacatttccttctttgaagaatgaaacaaatgatgatagatggaaagggtttatctatgctttgcaaggaatttatgataaagaaaatgcattgaagaagattagaatgttggaaggttttgctaatggaaactcattcagtaatctcttatggtggatccatagcagataa

SEQ ID NO: 5 - DNA, 1992 bp, Medicago sativa
  source 1..1992: mol_type = genomic DNA
atgtcttcatcatcttctcttcctttcctatttcctcaaactcattcaactgtcctcccaaacccttcaaacttcttctcacaaaacctactatccacacccctccctacaaactctttcttccaaaactttgttctccacaatggtgaaacacctgaatacattcacccttacctcatcaaatcctcaaacttttcactctctgtttcctaccctcttctcctcttttcagcaacaatgttgtaccaagttttttcaccggatctcacaatttcatcctcacaaaaaactcacacaaatataccaaaaaatcatgttatctcatcacatagtgatcttggtgtgactcttgacattccctcttcaaatctaagattctttttggttagaggaagcccttttataactgcttcagttagaaaaccaacctcactttcaatcacaacacttcataacatagtttctttgtcttcttttgatgacaaaaataccaaatacacccttcacctcaacaacactcagcaatggatcatatacacttcttcacctataaaattcaaccatgatggttctgagattgtatccaatccattttccggtataattcatatcgtagttgttcctagttccaaatatgagaaaattcttgataaattgagctcttgttaccctgtctccggtgatgcaaacatcaagaatagatttcatttggagtataaatggaaaaagaaatgttctggagatttactcatgctagcacaccctcttcatgttaagcttctatcacaaagtaacaatgttaatgttactgttttgcatgatttgaagtatacaagtgttgatggtgatctcgttggtgttatcggagattcatggatattgaaaactgatcctgttaatgtaacatggtattctagtaaaggtgttacaaaagaatcacatgatgagattgtttcagctcttgttaacgatgtgaaagagctgaattcttcagcaataacaacaaatggatcttatttttatggtaagatagtttcaagagctgcaaggtttgctttgatagctgaagaagtttcttaccctaaagtgattccaattatcaagaattttttgaaggaaactattgagctatggttgaatggaactttcaaagggaatggttttttgtatgaaaaaaaatggggtggattagttactaaacaaggggttaataattcaggtgttgattttggttttggaatttataatgatcatcattatcatttagggtattttctttatggaattgcagttcttgcaaagattgatccattttggggacaaaagtataaaccacaaatttatgcacttgtgaaagattttatgaacttgggccaaagggataacaaaaactatccaactttaaggtgttttgattttttcaagttgcattcttgggctgcaggagtgactgaatatgaaaatggaaggaatcaagaaagttcaagtgaagctgtgaatgcatattattcagcagcattgataggtctagcatatggtgacaaagatcttgttgctattggatcaacacttttagcatttgaaatcaatgctacacaaacttggtggcatgtgaaagttgaaaataatttatatggagaagagtttgcaaaagaaaataggattgttggtattttgtgggctaataagagagatagtaaactttggtgggctccttctgaatgtagagggtgtagggttagtatccaagttatgcctttgttgcctattactgagacattgttcaatgatggtgtttatgctaaggaattagtggaatggacactaccttctttgaagaatgaaacaaatgatgatagatggaaagggtttatctatgctttgcaaggaatttatgataaaggaaatgcattgaagaatattagaatgttggaaggttttgctaatggaaactcattcagtaatctcttatggtggattcatagcagataa

SEQ ID NO: 6 - DNA, 1983 bp, Medicago sativa
  source 1..1983: mol_type = genomic DNA
atgtcttctgttccattcctatttcctcagactcattcaactgtcctcccaaacccttcaaacttcttctcacaaaacttactatccacacccctccctacaaattcattcttccaaaactttgttctccaaaatggtgatcaacatgaatacattcacccttaccttgtcaaatcctcaaacttttccgtatctgtttcataccctcttctcctcttttcaacagcaatgttgtaccaagttttttcaccagatcttacaatctcatcctcacaaaaaactcacacaaacatacctaaaaaccatgttatttcatcatatagtgatcttggtgtgactcttgacattccctcttcaaacctaagattctttttggttagaggaagcccttttataactgcttcagttacaaaaccaacacctctttcaatcacaacaattcatagtataatttctttgtctccttttgataagaaaaaaaccaaatatactcttcaactcaacaacaatcagacatggatcatatacacttcttcaccaatcaacttgaaccatgatggttccgaggttaagtccggtccattttccggtattattcgtatcgcggttgttcctgattccaatggtgagaaaattcttgataaattcagctcttgttaccctgtctctggtgatgcaaacatcaagaagaaatttggtttggtttataaatggcaaaggaaaaattctggtgatttactcatgctagcacaccctcttcatgttaagcttttatcaaaaagtaacaatcatggtgttactgttcttgatgattttaagtataagagtgttgatggtgatcttgttggtgttgttggaaattcatggaatttgaaaactgattctgttaatgtaacatggcattcaaacaaaggtgttacaaaagaatcacatgctgaaattgtttctgctcttgttaatgatgtgaagaagctaaacttttcgtcgataacaacaaattcatcttatttttatggtaagattgttggaagagctgcaaggtttgctttcatagctgaagaagtttcttaccctaaagtgattccaattatcaagaattttttgaaggaaactattgagccatggttggatggaaatttcaaagggaatggttttttctatgaaaaaagttggggtggatttgttactcaacaagggattaatgattcaagtgctgattttggttttggaatttataatgatcatcattatcatttaggttattttctttatggaattggagttcttgcaaaaattgatccatcttggggacaaaagtataaacctcaagtttattcacttgtgaaagattttatgaacttgggccaaagggataacaaaaattatccaactttaaggtgttttgatccatacaagttgcattcttgggcttcggggttgaccgaatttgaacatggaaggaatcaagaaagttcgagcgaagctgtgaatgcgtactattcagtagcattggttggtttggcatatggcgacaaagatcttgtcgccactggatcaacgcttttagcgttggaaattaatgccgtgcaaacttggtggcatgtgaaattcgaaaataatttgtatggtggagatttcgcaaaagggaatcggatagtgggaattttatggtcaaacaaaagagatagtgcattatggtgggctgcatctgaatgtagagagtgtaggcttagtatacaagttttgcctttgttgcctataactgagtctttgttcaatgatggtgtttatgctaaggagcttgtggaatggacagtgccttctttcaagaacaagactaatattgaagggtggaaagggtttacctatgctttgcaaggtgtttatgataagaagaatgcattgaagaatattagaatgttgaaaggttttgatgatggtaactcttttagtaatatgttatggtggattcatagtaggtaa

SEQ ID NO: 7 - DNA, 1983 bp, Medicago sativa
  source 1..1983: mol_type = genomic DNA
atgtcttctgttccattcctatttcctcaaactcattcaactgtcctcccaaacccttcaaacttcttctcacaaaacttactatccacacccctccctacaaattcattcttccaaaactttgttctccaaaatggtgatcaacatgaatacattcacccttaccttgtcaaatcctcaaactcttccctatctgtttcataccctcttctcctcttttcaacagcaatgttgtaccaagttttttcaccagatcttacaatctcatcctcacaaaaaactcacacaaacatacctaaaaaccatgttatttcatcatatagtgatcttggtgtgactcttgacattccctcttcaaacctaagattctttttggttagaggaagcccttttataactgcttcagttacaaaaccaacacctctttcaatcacaacaattcatagtataatttctttgtctccttttgataagaaaaaaaccaaatatactcttcaactcaacaacaatcagacatggatcatatacacttcttcaccaatcaacttgaaccatgatggttccgaggttaagtccggtccattttccggtattattcgtatcgcggttgttcctgattccaatggtgagaaaattcttgataaattcagctcttgttaccctatttctggtgatgcaaacatcaagaagaaatttggtttggtttataaatggcaaagaaaaaactctggtgatttactcatgcttgcacaccctcttcatgttaagcttttatcaaaaagtaacaatcatggtgttactgttcttgatgatttcaagtataaaagtgttgatggtgatcttgttggtgttgttggaaattcatggaatttgaaaactgattctgttaatgtaacatggcattcaaataaaggtgttacaaaagaatcacatgctgaaattgtttctgctcttcttaatgatgttaagaagttaaacatttcgtcgataacaacaaattcatcttatttttatggcaagattgttggaagagctgcaaggtttgctttaatagctgaagaagtttcttaccctaaagtgattccaataatcaagaattttttgaaggagactattgagccatggttggatggaaatttcaaagggaatggttttttctatgaaaaaagttggggtggattagttactcaacaagggattaatgattcaagtgctgattttggttttggaatttataatgatcatcattatcatttaggttattttctttatggaattggagttcttgcaaaaattgatccttcttggggacaaaagtataaaccacaagtttattcacttgtgaaagattttatgaacttgggccaaagggataacaaaaattatccaactttaaggtgttttgatccatacaagttgcattcttgggcttcggggttgaccgaatttgaacatggaaggaatcaagaaagttcgagtgaagctgtgaatgcgtactattcagtagcattggttggtttagcatatggcgacaaagatcttgtcgccactggatcaacgcttttagcgttggaaattaatgccgtgcaaacttggtggcatgtgaaagtcgaaaataatttatatggtcaagatttcgcgaaagagaatcggatagtgggaattttgtgggctaacaaaagagatagtgcactatggtgggctgcatctgaatgtagagagtgtaggcttagtatacaagttttgcctttgttgcctataactgagtctttgttcaatgatggtgtttatgctaaggagcttgtggaatggacagtgccttctttcaagaacaaaactaatattgaaggttggaaagggtttacctatgctttgcaaggtgtttatgataagaagaatgcattgaagaatattagaatgttgaaaggttttgatgatggaaactcttttagtaatatgttatggtggattcatagtaggtga

SEQ ID NO: 8 - DNA, 1755 bp, Medicago sativa
  source 1..1755: mol_type = genomic DNA
atgttataccaagttttttcaccagatgttacaatttcttcctcacaaaaaactcacacaaacataccaaaaaaccatgttatctcatcatatagtgatcttggtgtgactcttgacattccctcttcaaacctaagattctttttggttagaggaagcccttttataactgcttcagttacaaaaccaacacctctttcaatcacaacaattcatagtataatatctttgtctccttttgataagaaaaaaaccaaatatacccttcaactcaataacaatcagacatggatcatatacacttcttcaccaatcaacttcaaccatgatggttccgaggttaaatccggtccattttccggtataattcgtatcgcggttgttcctgattccaatggtgagaaaattcttgataaattcagctcttgttatcctgtttctggtgatgcaaacatcaagaagaaatttggtttggtttataaatggcaaagaaaaaattctggtgatttactcatgctagcacaccctcttcatgttaagcttttatcaaaaagtaacaatcatggtgttactgttcttgatgatttcaagtataaaagtgttgatggtgatcttgttggtgttgttggaaattcatggaatttgaaaactgattctgttaatgtaacatggcattcaaacaaaggtgttacaaaagaatcacatgctgaaattgtttctgctcttgttaatgatgtgaagaagttaaacttttcgtcgataacaacaaattcatcttatttttatggtaagattgttggaagagctgcaaggtttgctttaatagctgaagaagtttcttaccctaaagtgattccaattatcaagaattttttgaaggagactattgagccatggttggatggaaatttcaaagggaatggttttttctatgaaaaaagttggggtggattagttactcaacaagggattaatgattcaagtgctgattttggttttggaatttataatgatcatcattatcatttaggttattttctttatggaattggagttcttgcaaaaattgatccttcttggggacaaaagtataagccacaagtttattcacttgtgaaagattttatgaacttgggccaaagggataacaaaaattatccaactttaaggtgttttgatccatacaagttgcattcttgggcttcggggttgaccgaatttgaacatggaaggaatcaagaaagttcgagcgaagctgtgaatgcgtactattcagtagcattggttggtttagcatatggcgacaaagatcttgtcgccactggatcaacgcttttagcgttggaaattaatgccgtgcaaacttggtggcatgtgaaagtcgaaaataatttatatggtcaagattttgcgaaagagaatcggatagtgggaattttgtgggcaaacaaaagagatagtgcactatggtgggcttcatctgaatgtagagagtgtaggcttagtatacaagttttgcctttgttgcctataactgagtctttgttcaatgatggtgtttatgctaaggagcttgtggaatggacagtgccttctttcaagaacaaaactaatattgaaggttggaaagggtttacctatgctttgcaaggtgtttatgataagaagaatgcattgaagaatattagaatgttgaaaggttttgatgatggaaactcttttagtaatatgttatggtggattcatagtaggtga

SEQ ID NO: 9 - DNA, 1755 bp, Medicago sativa
  source 1..1755: mol_type = genomic DNA
atgttataccaagttttttcaccagatcttacaatctcatcctcacaaaaaactcacacaaacatacctaaaaaccatgttatttcatcatatagtgatcttggtgtgactcttgacattccctcttcaaacctaagattctttttggttagaggaagcccttttataactgcttcagttacaaaaccaacacctcttttgatcacaacaattcatagtataatttctctgtctccttttgataagaaaaaaaccaaatacacccttcaactcaataacaatcagacatggatcatatacacttcttcaccaatcaacttcaaccatgatggttctgaggttaaatccggtccattttccggtattattcgtatcgcggttgttcctgattccaatggtgagaaaattcttgataaattcagctcttgttaccctatttctggtgatgcaaacatcaagaagaaatttggtttggtttataaatggcaaagaaaaaattctggtgatttactcatgctagcacaccctcttcatgttaagcttttatcaaaaagtaacaatcatggtgttattgttcttgatgattttaagtataaaagtgttgatggtgatcttgttggtgttgttggaaattcatggaatttgaaaactgattctgttaatgtaacatggcattcaaacaaaggtgttacaaaagaatcacatgctgaaattgtttctgctcttgttaatgatgtgaagaagctaaacttttcgtcgataacaacaaattcatcttatttttatggtaagattgttggaagagctgcaaggtttgctttaatagctgaagaagtttcttaccctaaagtgattccaattatcaagaattttttgaaggaaactattgagccatggttggatggaaatttcaaagggaatggttttttctatgaaaaaagttggggtggattagttactcaacaagggattaatgattcaagtgctgattttggttttggaatttataatgatcatcattatcatttagggtattttctttatggaattggagttcttgcaaaaattgatccttcttggggacaaaagtataaaccacaagtttattcacttgtgaaagattttatgaacttgggccaaagggataacataaattatccaactttaaggagttttgatccatacaagttgcattcttgggcttcggggttgaccgaatttgaacatggaaggaatcaagaaagttcgagtgaagctgtgaatgcgtactattcagtagcattggttggtttagcatatggcgacaaagatcttgtcgccactggatcaacgcttttagcgttggaaattaatgccgtgcaaacttggtggcatgtgaaagtcgaaaataatttatatggtcaagatttcgcgaaagagaatcggatagtgggaattttgtgggcaaacaaaagagatagtgcactatggtgggcttcatctgaatgtagagagtgtaggcttagtatacaagttttgcctttgttgcctgtaactgagtctttgttcaatgatggtgtttatgctaaggagcttgtggagtggacagtgccttctttcaagaacaaaactaatattgaaggttggaaagggtttacctatgctttgcaaggtgtttatgataagaagaatgcattgaagaatattagaatgttgaaaggttttgatgatggtaactcttttagtaatttgttatggtggattcatagtaggtaa

SEQ ID NO: 10 - DNA, 1977 bp, Pisum sativum
  source 1..1977: mol_type = genomic DNA
atgtcttctcctccttacctatttcctcaaactcaatcaaccattctcccaaacccttccaacttcttctccccaaacttgctatccacacccctccctaccagctctttcttccaaaactttgctctaaaaaatggcgaccaacctgaatacattcacccttacctcatccaatcctcaaactcttcactctcggtttcataccctttactcctcttctccacagcattactctaccaggttttctcaccagatctcactatctcatccacacaaaaacctcaaacaaacataccacaaaacaaccatgttatatcctcgtacagtgatcttggtgtgacgcttgatattcccactgcaaatctaagattttttctagttagaggaagcccttttgtaactgctttagttacaaaaccaacacctctttcaatcaaaacaaatcacaccattgtttctttctcatcatttgattacaagaaaaccaaatatagactatcactcaacaatggtcagaaatggatcatatacacttcttcaccaattaacttcaaccatgacggttcagaggttaagtccgatccgttttctggtataatccgtttcgccgttgtttctaattcgaataatgagaaaattctccatgaattcagctcgagttaccccgtttccggctatgcaaagatcgaggataaattcggtttggtttataaatggaaaactaaaaattccggtgatttactcatgctagcacatcctcttcatgttaagcttttgtcgaagaatagtaacgatcataaagttactattttgaatgattttaagtatagaagcgttgatggtgatcttgttggtgttgttggaaaatcatggttgttgaaaactgattctgttaatgtaacatggcattctagtaaaggtgtttcaaaagattcatacgaggaagttgtttctgctcttgagaaagatgtgaatgagttgaacgttgcgacgataaatacaacttcgtcttatttttacggcaagattgttgcaagagctgcaaggcttgctttgatagctgaagaagtttcttatgagaaagtgattccaattgttaaggattttttgaagaaaactattgagccatggttagatggaaacttcaaagggaatggttttttgtatgagaaaacatggggtggattggttactcaacaaggggttaatgatagtggtgctgattttggttttggtgtttataacgatcatcattatcatttaggttattttctttatggaattggagttcttgcaaaacttgatcaagattggggacaaaagtataaaccaatagtttattcacttttgaaagattttatgaacttgggtcaaagggataacaaaaactatccaactttaaggagttttgatccttacaagttacattcttgggcttcggggttgaccgaattcagagacggaaggaatcaagaaagtacaagcgaagctgtgaacgcgtactactcggttaccttagtaggtttagcttatgacgatgaagatttggtcgcgatcggatcgacgcttttagcgttcgaaattaacgcggcgcaaacttggtggcacgtgaaagccgagaacaatgtgtatggtactgattttgctaagcaaaatccggtagttggtgttttgtgggcgaacaagagagatagtagtctttggtgggcttcgtcggagtgtcgccagtgtcggcttagtatacaagttttgcctttgttgcctataactgagaatttgttcaatgatggtgtttatgctaaggagcttgtggaatggacatggccaactttgagtaaagaagggtggaaagggtttacttatgctttgcaaggtgtttatgataaggaaaatgctttgaagaatattagaactttgaaaggttttgatgatggaaactctttgagtaatttgttatggtggattcatagtagatga

SEQ ID NO: 11 - DNA, 1983 bp, Pisum sativum
  source 1..1983: mol_type = genomic DNA
atgtgttctcctccttacctatttcctcaaactcaatcaaccattctcccaaacccttccaacttcttctcccaagacttactatccacacccctacctacaaactctttcttccaaaactttgctctcaaaaatggtgaccaacctgaatacattcacccttacctcatccaatcctcaaactcttcactctcggtttcattccctctgctcttcttctccacagcattgctctaccaggttttcacaccagatctcactatctcatccacacaaaaacctcaaacaaacataccacaaaacaaccatgttatatcctcgtacagtgatcttggtgtgacgcttgatattcccactacaaatctaagattttttttggttagaggaagcccttttgtaacagctcaagttacaaaaccaacacctctttcaatcaaaacaattcacgccattctttctttctcatcatttgataacaaaaaaaacaaatatgcactttcactcaacaatggtcagaaatggatcatatacacttcttcaccaattaacttcaaccatgatatttccgaggttaaatccgatccgtttaccggtgtaatccgtatcgcagctgtttctgattcgaataacgagaaaattctcgacgaattcagctcgagttatcccgtttccggccatgcaattgtggacgtaaagaataaatttggtttggtttataaatgggagactgaaaattccggtgatttactcatgttagcacaccctcttcatgtcaagcttttgtcgaagaatagtaacgatcataaagttactattttgaatgattttaagtatagaagcgttgatggtgatcttgttggtgttgttggaaattcatggttgttgaaaactgatactattaatgtaacatggcattctagtaaaggtgttgcaaaagaatcatatgaggaagttgtttctgctcttgagaaagatgtgaatgagttgaacgttgcgtcgataagtacgacttcgtcttatttttacggtaagattgttgcaagagctgcaaggtttgctttgatagctgaagaagtttcttatgagaaagtgattccaattgttaaggattttttgaagaaaactattgagccatggttagatggaaacttcaaagggaatggttttttgtatgagaaaacatggggtggattggttactcaacaaggggttaatgatgctggtgctgattttggttttggtgtttataatgatcatcattatcatttaggttattttctttatggagttggagttcttgcaaaacttgatcaagattggggacaaaagtataagccaatagtttattcccttttgaaagattttatgaacttgggtcaaggggataacaaaaattatccaactttaaggagttttgatccttacaagttgcattcttgggcttccgggttgaccgaattcagtgatggaaggaatcaagaaagtacgagtgaagctgtgaatgcgtattattcagctgcattggtaggtttagcttatggtgacgaagatctggttgcgattggatcgacgcttttagcattggaaattaacgcggcacaaacttggtggcatgtgaaaaccgagaataatgtgtatggtgccgattttgctaaacaaaattcggtagttggtgttttgtgggcgaacaagagagatagtagtctttggtgggcttcttcggaatgtcgcgaatgtcgacttagtatacaagttttgcctttgttgcctataactgagaatttgttcaatgatggtgtttatgttaaggagcttgtggagtggacatggccaactttgagtaatgaagggtggaaagggtttacttatgctttgcaaggtgtttatgataaggaaaatgctttaaataatattagagctttgaaaggttttgatgatggaaattctttgagtaatattttatggtggattcatagtagatga

SEQ ID NO: 12 - DNA, 1965 bp, Vicia faba
  source 1..1965: mol_type = genomic DNA
atgtcttctcctccttacctatttcctcaaactcaatcaaccattctcccaaacccttccaacttcttctcccaaaacttgctatccacacccctccctacaaactctttcttccaaaactttgctctcaaaaatggtgaccaacctgaatacattcacccttacctcatccaatcctcaaactcctccctctcagtttcataccctttactcctcttctcaacagcattgctctaccaggttttctcaccagatctcactatctcatccacacaacaacctcaaacaaacataaaccatgttatatcctcgtacagtgatcttggtgtgactcttgatattcccacttcgaatctacgattttttctcgttagaggaagtccttttgtaactgctctagtcacaaaaccaacacctctttcaatcaaaactattcacaccattgtttctttctctacattcgataacaagaaaaccaaatatacactttcactcaacaatactcagaaatggatcatatacacttcttcaccaattaacttcaaccatctcggttccgaggttatatccgatccattttccggtataattcgtattgcaagtgtttctaattcgaataatgagaaaattctcgatgaattcagctcgagttatccggtttcgggctatgcgaagatcgagaataaattcggtttagtttataaatgggagactcaaaattccggtgatttactcatgctagcacaccctcttcatgctaagcttttgtctaatagtaaagatcataaggttactattttgaacgattttaagtatagaagcattgatggtgatcttgttggtgttgttggaaattcatggttgttgaaaaccgattcttttaatgtaacatggcattctagtaaaggtgttacaaaagaatcatacgaggaagttgtttctgctcttgagaaagatgttaatgagttgaatgttgcgtcgattacgacgacttcgtcgtatttttatggtaagattgttgcaagagctgcaaggtttgctttgatagctgaagaagtttcttatgagaaagtgattccggttgttaagggttttttgaagcaaactattgagccatggttagatggaaagttcaaagggaatggttttttgtatgagaaaacttggggtggattggttactcaacaaggggtgaatgatgttggtgctgattttggttttggtgtttataatgatcatcattatcatttaggttattttctttatggaattggagttcttgcaaagattgatcaagattggggacaaaagtataagccaatagtttattcacttttgaaagattttatgaacttgggtctaggggataatccaaactatccaactttaaggaattttgatccttacaagttacattcttgggcttcggggttgaccgaattcagagacggaaggaatcaagaaagtacgagtgaagctgtgaatgcgtattattcagttacgttaataggtttagcttatggtgacgaagatctggttgtggttggatcgacacttttagcgttggaaattaacgcggcgcaatcttggtggcatgtgaaagccgagaacaatgtgtatggtactgattttgctaaacaaaatccgattgtcggagttttgtgggcgaacaagagagacagtagtctttggtgggcttcgtcggcgtgccgtgaatgtcggcttagtatacaagttttgccattgttgcctatcactgagaatttgttcaatgatggtgtttatgctaaggagcttgtggaatggacattgccaactttgagtaatgaagggtggaaagggtttacttatgctttgcaaggtgtttatgataaggaaaatgcattgaagaatattagaactttgaaaggttttgatgatggaaactctttgagtaatttgttatggtggattcatagtagatga

SEQ ID NO: 13 - DNA, 1986 bp, Trifolium pratense
  source 1..1986: mol_type = genomic DNA
atgtcttctgttcctttcctatttcctcaaactcattcaactgttcttccaaacccttcaaatttcttctcacaaaacttactatccacacccctccctactaactctttcttccaaaactttgttctcaaaaatggtgaccaacctgaatacattcacccttatctcatcaaatcctcaaacttttcactttctgtttcatacccttttctcctattttcaacagcaatgttgtaccaagttttctcaccagatctcactatttcatcctcacaaaaatctcacacaaactcacaaaaaaataagcattttatctcatcctatagtgatcttggcgtgactcttgatattccatcttcaaatctaagattctttcttgttagaggaagtccttttgtaactgcttctgttacaaaaccaacacctctttcaatcacaacattgcataacatagtttctttgtcttgttttgataacaaaaaaaccaaatatacacttttgctcaacaatactcagaaatggattatatacacttcttcaccaatcaatttaaaccatgatggttccgaggtgaaatccggtccattttcggggataattcgtatcgcggttgttcctgattcgaattacgagaagattctcgataaattcagctcttgttaccctgtttctggctatgcaaacattcagaagaaatttggtttggtttataaatggcaaaggaaaaattcaggtgatttacttatgctagcacaccctcttcatgttaagcttttatcaaaaagtaacaatcatggtgttactgttttgaatgattttaagtatagaagtgttgatggtgatcttgttggtgtggttggaaattcatggaatttgaaaactgatcctattgatgtaacatggcattctagtaaaggtgttacaaaagaatcacatgatgaaattgtttcagcacttgttaaagatgtgaagaaattgaatatttcagcaatagaaacaaattcatcttatttttatggtaagattgttggaagagctgcaagatttgctttgatagctgaagaaatttcttattttaaagtgattccaattattaagaattttttgaagaaaactattgagccatggttagatggtaatttcaaagggaatggttttttttatgaaaaaagttggggtggattagttactcaacaagggattaatgattcaagtgctgattttggttttggagtttataatgatcatcattatcatttaggatattttctttatggaattggggttcttgcaaaaattgatcctttatggggacaaaagtataaaccaatagtttattcacttttgaaagattttatgaacttgggcaaaagagataacaaaaattatccaactttaaggtgttttgatccatacaagttacattcttgggcttccggggtgactgaatttgaaaatggaaggaatcaagaaagttcgagcgaagctgtgaatgcttattattcggccgcattagtaggtctagcttacaatgacaaaaatcttgttgctaccggatctacgcttttagcattggaaattaatgccgtgcaaacttggtggcatgtgaaagccgaaagtaatttgtatggtgaagattttgcgaaagaaaataggattgttggtattttgtgggcgaataaaagggatagtaaactatggtgggcaccatccgagtgtcgagagtgtaggcttagtatacaagttttacctttgttgcctattaccgagactttgttcaatgacggtgtttatgctaaggagcttgtggagtggacattgccatctttgaagaataagactaatgttgaaggatggaaagggtttacctatgctttgcaaggtgtttatgataacaaaaatgcattgaagaaaattaggttgttgaaaggttttgatgatggaaactcttttagtaatctattatggtggattcatagtaggtga

SEQ ID NO: 14 - DNA, 1986 bp, Trifolium repens
  source 1..1986: mol_type = genomic DNA
atgtcttctgttcctttcctatttcctcaaactcattcaactgttcttccaaacccttcaaatttcttctcacaaaacttactatccacacccctccctactaactctttcttccaaaactttgttcttaacaatggtgaccaacctgaatacattcacccatatctcatcaaatcctcaaactcttcactttctgtttcatacccttttctcctattttcaacagcaatgttgtaccaagttttctcaccagatctcaccatttcatcctcacaaaaatctcactcaaactcaccaaaaaataagcatgttatctcatcctatagtgatcatggtgtgactcttgatattccatcttctaatctaagattctttcttgttagaggaagtccttttgtaacagcttatgttacaaaaccaacacctctttcaatcacaacattgcataacatagtttctttgtcttcttttgataacaaaaaaaccaaatttactcttttgctcaacaatactcagaaatggatcatatacacttcttcaccaatcaatttaaaccatgatggttccgaggttaaatccgatccattttcggggattattcgtatcgcagttgttcctgattcgaattacgagaaaattctcgataaattcagctcttgttaccctgtttctggctatgcaaacattcagaagaaatttggtttggtttataaatggcaaacaaaaaattcaggtgatttacttatgctagcacaccctcttcatgttaagcttttatcaaaaagtaacaatcatggtgttattgttttgaatgattttaagtatagaagtgttgatggtgatcttgttggtgttgttggaaattcatggaatttgaaaactgattctattgatgtaacatggcattctagtaaaggtgttacaaaagaatcacatgatgaaattgtttcagcacttgttaaagatgtgaaggaattgaatatttcatcaatagcaacaaattcatcttatttttatggtaagattgttggaagagctgcaagatttgcattgatagctgaagaagtttcttattttaaagtgattccaattattaagaattttttgaaggaaactattgagccatggttagatggaaatttcaaagggaatggttttttttatgaaaaaagttggggtggattagttactcaacaagggattaatgattcaagtgctgattttggttttggagtttataatgatcatcattatcatttagggtattttctttatggaattggggttcttgcaaaaattgatcctttatggggacaaaagtataaaccaagagtttattcaattttgaaagattttatgaacttgggccaaagggataacaaaaattatccaactttaaggtgttttgatccatacaaattgcattcttgggcttccggtgcgactgaatttgaaaacggaaggaatcaagaaagttcgagtgaagctgtgaatgcatactattcggccgcattagtaggtctagcatacaacgacaaaaatcttgttgctaccggatctacgcttttagcattggaaattaatgccgcgcaaacttggtggcatgtgaaagttgaaaataatttgtatggtgaagattttgcgaaagaaaataggattgttggtattttgtgggcgaataaaagggacagtaaattatggtgggcaccatccgagtgtcgagagtgtaggcttagtatacaagttttacctttgttgcctattaccgagactttgttcaatgatggtgtttatgcgaaggagcttgtggagtggacattgccatctttgaagaataagactaatgttgaaggatggaaagggtttacctatgctttgcaaggtgtttatgataagaaaaatgcattgaagaagattaggatgttgaaaggttttgatgatggaaactcttttagtaatctattgtggtggattcatagtaggtga

SEQ ID NO: 15 - DNA, 1437 bp, Trifolium repens
  source 1..1437: mol_type = genomic DNA
atgtcttctgttcctttcctatttcctcaaactcattcaactgttcttccaaacccttcaaatttcttctcacaaaacttactatctacacccctccctactaactctttcttccaaaactttgttcttaacaatggtgaccaacctgaatacattcacccatatctcatcaaatcctcaaactcttcactttctgtttcatacccttttctcctattttcaacagcaatgttgtaccaagttttctcaccagatctcaccatttcatcctcacaaaaatctcactcaaactcatcaaaaaataagcatgttatctcatcctatagtgatcttggtgtgactcttgatattccatcttcaaatctaagattctttcttattagaggaagtccttttgtaacagctttagttacaaaaccaacacctctttcaatcacaacattgcataccattgtttctttgtcttcttttgataacaaaaaaaccaaatttactcttttgctcaacaatactcagaaatggatcatatacacttcttcaccaatcaatttaaaccatgatggttccgaggttaaatccgatccattttcggggattattcgtatcgcagttgttcctgattcgaattacgagaaaattctcgataaattcagctcttgttaccctgtttctggctatgcaaacattcagaagaaatttggtttggtttataaatggcagacaaaaaattcaggtgatttacttatgctagcacaccctcttcatgttaagcttttatcaaaaagtaacaatcatggtgttattgttttgaatgattttaagtatagaagtgttgatggtgatcttgttggtgttgttggaaattcatggaatttgaaaactgattctattgatgtaacatggcattctagtaaaggtgttacaaaagaatcacatgatgaaattgtttcagcacttgttaaagatgtgaaggaattgaatatttcatcaatagcaacaaattcatcttatttttatggtaagattgttggaagagctgcaagatttgcattgatagctgaagaagtttcttattttaaagtgattccaattattaagaattttttgaaggaaactattgagccatggttagatggaaatttcaaagggaatggttttttttatgaaaaaagttggggtggattagttactcaacaagggattaatgattcaagtgctgattttggttttggagtttataatgatcatcattatcatttagggtattttctttatggaattggggttcttgcaaaaattgatcctttatggggacaaaagtataaaccaagagtttattcaattttgaaagattttatgaacttgggccaaagggataacaaaaattatccaactttaaggtgttttgatccatacaagttacattcttgggcatcc

SEQ ID NO: 16 - DNA, 1986 bp, Trifolium subterraneum
  source 1..1986: mol_type = genomic DNA
atgtcttctgttcctttcctatttcctcaaactcattcaactgtccttccaaacccttcaaatttcttctcacaaaacttactatccacacccctccctactaactctttcttccaaaactttgttctcaacaatggtgaccaacctgaatacattcacccttatctcatcaaatcctcaaactcttcactttctgtttcatacccttttctcctattttcaacagcaatgttataccaagttttttcaccagatctcaccatttcatcttcacaaaaatctcactcaaactcaacaaaaaataagcattttatctcatcctatagtgatcttggtgtaactcttgatattccatcttcaaatctaagattctttcttgttagaggaagtccttttgtaacagcttctgttacaaaaccaacacctctttcaatcacaacattgcataacatagtttctttgtcttcttttgataacaaaaaaaccaaatatactcttttgctcaacaatacccagaaatggattatatacacttcttcaccaatcaatttaaatcatgatggttccgaggttaagtccgatccattttcggggataattcgtttcgcggttgttcctaattcgaattacgagaagattctcgataaattcagctcttgttacgctgtttcgggatatgcaaatattcagaagaaatttggtttggtttataaatggcaaaggaaaaactcaggtgaattacttatgctagcacatcctcttcatgttaaacttttatcaaaaagtaacaatcatggtgttactgttttgaatgattttaagtatagaagtgttgatggtgatcttgttggtattgttggaaattcatggaatttgaaaactgattctattgatgtaacatggcattctagtaaaggtgttacaaaagaatcacatgatgaaattgttgcagcacttgttaaagatgtgaaggaattgaatatttcagcaatagaaacaaattcatcttatttttatggtaagattgttggaagagctgcaagatttgctttgatagctgaagaagtttcttattttaaagtgattccaattattaagaattttttgaagaaaactattgagccatggttagatggaaattttaaaggaaatggttttttttatgaaaaaagttggggtggattagttactcaacaagggattaatgattcaagtgctgattttggttttggagtttataatgatcatcattatcatttagggtattttctttatggaattggggttcttgcaaaaattgatcctttatggggacaaaagtataaaccaatagtttattcacttttgaaagattttatgaacttgggccaaagggataataaattttatccaactttaaggtgttttgatccatataagttgcattcttgggcatccggggtgactgaatttgaaaacggaaggaatcaagaaagttcgagcgaagctgtgaatgcgtactattcggccgcattagtaggtctagcgtacaatgacaaaaatcttattgctaccggatctacgcttttagcattggaaattaatgccgcgcaaacatggtggcatgtgaaagtcgaaaataatttatatggtgaagattttgcgaaagaaaataggattgttggtattttgtgggcgaataaaagggacagtaaactatggtgggcagcatccgagtgtcgagagtgtaggcttagtatacaagttttaccgttgttgcctattacggagactttgttcaatgatggtgtttatgctaaggagcttgtggattggacattgccatctttgaagaataagactaatgttgaagggtggaaagggtttacctatgctttgcaaggtgtttatgataagaaaaatgcattgaagaagattaggatgttgaaaggttttgatgatggaaactcttttagtaatctattgtggtggattcatagtaggtga

SEQ ID NO: 17 - DNA, 2172 bp, Lotus japonicus
  source 1..2172: mol_type = genomic DNA
atgatttttattacaaataacggttccaaaggcaacacatatgcaagaagcttcattctaacttctaaggtcaactttcaccaatcctctctagtctctaatctcaccaaaaactcatataagaagacaacaccaccacacaaacaattgcaccaacatctcatcttctctccaccaacaacaatgcctccttcttctcctttcctcttccctcaaacccaatccacagtcctcccaaacccttcaaccttcttctcccaaaacctcctctcatctccactccccacaaactccttcttccaaaaccttgtcatccaaaatggttcccaacctgaatacattcacccttatctcatccaatcctcaaactcctccctttctgcctcatacccacttctcttcttctctgcagcactcttataccaaacctttgttccagatctcacaatctcttccactatcaaaacctcaaatcctcaaaaccatgtaatctcatcttacagtgaccttggtgtcacattagacatccccagttccaatttgagattctatctagccagaggaagcccttacataacagcctcagtgaccaaaccaacaccactttcaatcacaacagttcactccatagtgtctctgttatccgctgctgacaaaaccaagcacacccttcagctcaataacaatcagacatggctaatatactcttcagccccaatcaatttaaataaacatggaagctctgagcttcaatctgacccattttctggggtgattcgtatagctgttgttcctgattcaacttcaaaccctaagtatgaagaagttcttgacaagttcagttcttgctaccctgtttctggggatgcaaaactcaaggggaatttcaccgtggtgtataaatggcagaggaaaaattcaggggatttgctcatgctagctcaccctcttcatctcaagcttctctcaaaaaacaagcttgctgccactgttctctatgatttcaagtatagaagcgttgatggtgaccttgttggtgttgttggagattcatgggtgttggaagctgagcctgttcctgtaacatggcattctaacagaggaatcaaaaaagaatcttatggggaaattgtttcagcacttttgaaagatgtgaaggagctgaattattctgcagtggcaacaaattcttcttatttttatgggaagcttgttggaagagctgcaaggtttgcattgatagcagaagaagtttctttcccaaaagtgattccaaagattgtcaagtttctgaaggagagtattgagccatggttggatggaacattcaaaggaaatggctttctctatgagacaaaatggggtgggcttgttactcaacaagggtccaaagatgcaggtgctgattttgggtttgggatttacaatgatcaccatttccacttggggtactttctctatggaattgcagttcttgcaaagattgaccctgcttggggacagaaatacaagccccaagcctatgcacttgtgaatgatttcatgaacttgggacaaagatactacactttctctccgcggttacggtgtttcgatccttacaagatgcactcttgggcctcggggttaaccgagttcgaaaatgggaggaatcaggaaagtacaagtgaagctgtgaatgcttactactcagcagcattgatgggtctagcgtacggcgacacacgtctagctaccactggatcaacactcacggcactggaaattggtgctacacaaatgtggtggcatgtgaaaaaggaacaaattttgtacccagaagaatttgcagaagataacagaattgtggggattctttgggctaacaagagagacagtaatctatggtgggctcctgctgagtgcagagaatgcaggttaagtatccaagttctaccattgttgcctgttactgaatctttgttctctgatgctggttatgctaaggagcttgtggagtggacattgccttctttgaaaagcaaatcaaatgtagaagggtggaaggggtttacctattccttgcaagggatttatgataaggaaatagcattgaagagtataagaatgttgaaaggttttgatgatgggaactcatacagtaatctgttgtggtggattcatagcagataa

SEQ ID NO: 18 - DNA, 1992 bp, Lupinus angustifolius
  source 1..1992: mol_type = genomic DNA
atgtcttctcctccattcctcttctcccaaactcaatccacagtccttccaaatccatcaactttcttctcccaaaacctcctctcttctccactccctacaaactctttcttccaaaactttgttctcaaaaatggtgaccaacctgaatacattcacccttacctcatcaaatcctcaaactcttcactttctgtctcttacccatttctccttttcaccacagcaatgctttaccaagtttttgtgccagatcttacaatatccacatcatcatcatcacataaaagtgaaaccaaaactagccatgtaatttcatcttatagtgatcttggtgtcactttggatattccttcttcatatttaagattctttttagttagaggtagtccttttataacaacttctgttacaaaaccaaccactctttctataacaacaaccaataaaattgtctcattgcattcttttaatgacaaaaccaaacacacccttcaacttcaaaacaaccaaacatggcttatatacacttcttacccaattgtcttctatcacaaagactatgctattgaatcaaacaagttttcgggtattattcgattcgcggcctggcctgattccaccccgaaatatgaggaaattcttgacaagtttagttcttgttaccctgtatcaggtgatgcaacaattaagaatccgtttcgggttgtttataagtggcaaaggaaaaggagtggtgaattgcttatgttagctcaccctcttcatgttaagcttttatcatcatcattagcatttaacaatgttactgttttgaatgattttaagtatagaagtgtagatggtgatcttgttggtgttgttggtgattcttgggttttggaaacagaacatgttcctataacatggcattctaagaatggtgttaagaaagaatcatataatgagattgtttcagcactttttaaggatgttaaggagcttaatgcttctaatgtaacaacaaattcttcttacttttatggtaagcttgttggtcgagctgcgaggctcgcattgatcgcggaagaggtgtcttatctcgaagtgattccgaaaataagtgattttttgaaggagatgattcagccttggttggatgggaatttcaaagggaatggttttctatatgagagaaaatggggtggacttgttactaaacaagggtctatagatgcaggtgctgatttcgggttcggaatttacaatgatcatcattttcatttggggtatttcctttatggaattgcagtgcttgcaaagattgatccagcatggggtcaaaaatacaagcctcaagcttatgcacttgtcacagattttctgaacttaggacaaagattcaactcatattcgccgcgattaaggtgtttcgatttgtacaagttacactcttgggcttcagggataaccgaattcgaagacggaaggaatcaggagagtacaagtgaagctgtaaatgcatactatgcagcagcattactcggtctagcatatcgcgacacgcgactcgttgcgactgcatcgactcttacagcattggaaattctagcagcacaaacttggtggcatgtgaaatccgaagacaagttgtatgatgaagagtttacaaaagataacagaattgtgggtattttgtgggctaataagagagatagtaagctatggtgggcttcttcggaatgtagagagtgtagattaagtattcaagtgttgcctttggttcctgttactgaatcattgttctctgatgctggttatgtgaaggagcttgtggaatggactttaccttctttgaagaataaatcaaatgttgatgggtggaaagggtttacctatgcattgcaaggaatttatgataaagagaattcattgaagaagattagaatgttgaaaggttttgatgatggaaactcattcagtaatctcttatggtggattcatagcagataa

SEQ ID NO: 19 - DNA, 1971 bp, Lupinus angustifolius
  source 1..1971: mol_type = genomic DNA
atggctgctcctactcctttccttttccctgcaactcaacccacaatactccctgacccatcaacctttttctcttcaaacctttcatctccacttcctactaactctttcttccaaaactttgttcttaatagtggggaacaacctgaatatattcacccttatcttgtcaaatccacaaaaaactcactttctattgcataccctttgctccttttcactgcatcagtgttttaccaaacttttgcgcctgatctcactatatcttctgctacaccccaagaatctgccgcaaaaaaccatgttatctcatcctacagtgaccttggtgtcactttggacattccatcttcaaatttaagattctttctagttagaggaagcccttatataactgcttctgttactaaaccaaccactctttctatcaaaacaacttctcctatagaatccttaaatccatctaaggacaacaccaaatacattcttaaactgaaatccggtcagacatggataatatattcatcctccgctatcagtttaaccaagggggaaactgaaatcagctcaaactcattttctggtatcattcgattcgcgtcgttgcgtaatcctcaacaggagagtactcttgacaaatacagctccagttacccggtctcgggttatgcagtgttcaacaaatcgtttaatgtggtatataatttggaaaaggaagggaatggtgatttactcttgctagctcatcctcttcatgttaagcttctatcatcaaaatctaataaagttactgttctaagtgacttcaagtatccaagtgtagatggtgaacttgttggtgttgttggtgattcatgggagttagaaacaaaacatgttcctttaacatggaattccgtaaaaggtgtgaagaaagaagcatatgaagagattgttaaagcgcttgttaatgatgtgaatgagttaaactcatcaaatgtaacaacatcttcatcttacttctatggaaagcttgttgctagggctgcaaggcttgcattgatagcagaagaggtatctaacagtgaagtgattcccaaaatcactaagtttctgaaggatacgattcaaccttggttggatggtagtttcaaagggaatagttttctatatgaaaaaaagtggggtggacttgtaactaaacaagggtctacagataaaggtgctgattttggttttggggtttacaatgaccatcattatcatttggggtacttcatttatggaattgcagtgcttgcaaagattgatacagcatggggacagaagtacaaacctcaagcttatgcacttgtgtcagattttctgaacacagacctaaaatcaaactcacattatccacttttaaggaactttgacgtgtacaagttacactcttgggcttcagggttaactgaatttgcagatggaaggaatcaagaaagtacaagtgaagctgttaatgcttactatgcagcagcattgatgggtgtagcatatcatgacatggatctagttcgcattgcatcaactgtgacagcattggaaattcatgctgcacaaacatggtggcatgtgaaatctggagacaaattgtatgcagaagaatttgctaaagggaacaaaattgtgggtattgtatggtctaacaagagagatagtagtctatggtgggcttcagctgaagctaaagagtgtaggttaagtattcaagttttgcctttgtctcctattactgaagcattgttctctgatgctgcatatgtgaaggagcttgttgaatggactttaccttctttgaataaaccaaatatagaagggtggaaagggtttacctatgcattgcaagggatttatgataaaagtagttcattggagaagattagagcattgaaaggtgttgatgatgggaattcattcactaatctcttatggtggattcatagcagataa

SEQ ID NO: 20 - DNA, 2070 bp, Lupinus albus
  source 1..2070: mol_type = genomic DNA
atgcagcaaagcctatataaatccaaaaagtccccattgccattccatatgcatatcctctcctcaatttcaatggctcacaacctccaacatgaacctttcctcttcccactaacccattccacagtcctccctgacccttctaacttcttctcaccaaaccttctctcaactccactccctacaaactctttcttccaaaactttgctctcaaaaatggtgaccaacctgaatatattcacccttatctcatcaaatcctcaaactcttcactttctgtctcatacccttctcactttttcaccacagctttcatataccaagttttcattgctgatcttaccatatctgcttctgttaaaaccaactctgattctatacataagcatgttatctcttcctacaatgatcttagtgttacattggattttccttcttcaaatttgaggttctttcttgttaggggaagtccttttcttacagcaaatgttactagtagtacaccactttccattactactattcatgctatactttcattttcttcaagtgattctcttaccaagtttactcttaagcttaataatagccaaacatggcttatatattcctcttcaccaatgaaattcagtcacaccctctctggtattagttctgatgcattttgtggtgtgattcgtatagcagtgttgcctgagtcaaaaaattcaaaatttgaggaaatacttgataggttcagttcttgttaccctatttctggtgatgctatactcaaaaaaccattttctgttgtatataaatgggaaaagaaagggttaggtgatttgttactgttagcacatcctctgcatcttcaaatgttgtctaagaaaaattctgatgttactattcttgatgagtttaagtataaaagcattgatggggaccttgttggtgttgttggtgattcatggttattgaaaacaaaacctgtttatgtaacatggcattcaatacaaggtgtaaaaaaagaatcctatagtgaaattgtttcagcactttccaaagatgttgaaggtctaaattctgctgcaataacaacagcttcatcttactattatgggaaattggttgcaagggcagcaaggttggcattgatagctgaagagattggatttcgtgatgcgatttcggcgatcaccaagttcttaaaggaatcaattgagccatggcttgatggaactttagaagaaaatggttttctatatgatgaaaaatggggtggccttgttactaaacaagggtctattgattcaggtgctgattttgggtttggaatttacaatgatcatcattaccatcttgggtattttctatatggaattgcagtgcttgtgaaaattgacccatcatggggaattaagtataaacctcaagcatattcactaatgcaagattttatgaacctaggagaaaaatcaaactcaaattacccaactttaaggtgttttgatctatataaattgcattcttgggctggtgggttaactgaatttgcagatggaagaaatcaagagagtactagtgaagctataaatgcatattattcagcagctttattaggcctagcatataatgacactaatatttttgaaactgcatcaacttttgcatcattagaaattcatgcagctaagacatggtggcatgtgaaatttggtgataatctttatgaggaagattttacaaaagagaatagaataatgggtgttttatggtctaataaaagagatagtgggttatggtttgcacctcctgcaatgaaagagtgtagggttggaattcaactattaccattagtacctatttctgaaatgttgttttctaatgttagttttgtgaaggaacttgtgaagtggacattgctagctttggatagaaatgatgttgaagatgaatggaaagggtttgtttatgcattgcaaggaatttatgataatgaaagtgctttgcaaaagattagaagattgaaaggttttgatgatgggaactcattcactaatctcttatggtggattcatagcagatga

SEQ ID NO: 21 - DNA, 1971 bp, Lupinus albus
  source 1..1971: mol_type = genomic DNA
atgtctgttcctactcctttcctcttcccttcaattcaatcgacagtacttcctgacccatcaagctttttctccccaaacatttcatctccacttcctacaaactctttcttccaaaatcttgttctaaatggtggaggacaacctgagtattttcacccttatctcataaactccacaaaaacctctctttctgttgcataccctttgctccttttcactgcatcagtagtgtaccaaacttatgtgcctgaactcaccatatctgctacatcccaagaatctgccacaaaaaaccatgttatctcatccttcagtgaccttggtgtcactttggacctaccctcttcaaatttaagattctttttagttagaggaagcccttacataactgcttctgttactaaaccaaccactctttctatcaacacatcttctgctattgaatccttaagtgcatcatctcatcgcaacaccaaatacatccttaaactgaaatccaagcagacatggataatatattcatcctctcctatcagtttaactaatgagggaactgaaatcagatcaaactcatattctggtatcattcgattcgcgtcgttgcgtaatcctcactatgagagcactcttgataaattcagctcctcttacccggtctcgggtgatgcagagatcaagaaaccgtttcatttgagatataaatggcagaagaaagggaatggtggtttactcatgctagctcaccctcttcatgttaagcttctaccacgattatttagtcatgtcattgttctacgcgatttcaagtatccaagtgtagatggtgatcttgttggtgttgttggtgattcatgggagttagaaacaaaacctgttcctgtaacatggcgttcagtaaaaagtgtgaagaaagaatcatatcaagagattgttaaagcgcttgttaaagatgtgaacgagttaaactcatcaaatgtaacatcaacttcatcttacttttatggaaagcttgttgctagggctgcaaggcttgcattgatagcagacgaggtgaataatcatgaagtgattcccaaaatcagtatctttctgaaggagacgattcagccttggttggatggaagtttcaaagggaatgcttttctatatgaaaaaaggtggggtggacttgttactagacaagggtctgtagataaaggtgctgattttggttttggggtttataatgatcatcattatcatttggggtacttcctttatggaattgcagtgcttgcaaagattgatacagcatgggcaaagaagtataaatctcaaacttatgcacttgtgacagattttttgaacaccgaccaaagattaaaacaatctccacgtttaaggaattttgacttatacatgttacactcttgggcttcagggttaactgaatttggcgatggaaggaatcaagaaagtacaagtgaagctgtaaatgcttactatgcagcagcattggtgggtctagcatatggcgacaagcgtctcattagcactgcatcaaccctaacagcattggaaattcgtgctgcacaaacatggtggcatgtgaaatctaaaaacaaagtgtatgcagaagaatttgctaaagggaacaaaattgtgggtgttctttggtctatcaagagagacagtggtctatggtgggctgcagctgaacgtaaagagtgtaggctaagtattcaagttttgcctttgtcacctattactgagtcattgttctctgatccttcatatgtgaaggagcttgtggaatggactttaccttctgtggagagtaaacaaaatgttgaagggtggaaagggtttatctatgccttgcaagggatttatgataaaggaaaatcattggagaagataagaactttgaaaggtgttgatgatgggaattcattcactaatctcttatggtggattcatagcagatga

SEQ ID NO: 22 - DNA, 1971 bp, Vigna unguiculata
  source 1..1971: mol_type = genomic DNA
atgtcttcttcatcttcttttatgttccctcaaactcaatccacagttctcccagacccttcaacctacttctcctcaaaccttctttcatctccactccccacaaactctttcttccaaaactttgttctttcaaaaggatcacaacctgagtatattcacccatacctcattcaaacctcaaagtcctcactttctgcctcataccctcttctcttcttcactgcagcagtgttgtaccaaacttttgtgccggatctcacaatctcttccagtcaaacacttccaactcaacaaaaccatgtagtctcatcattcagtgaccttggtgtcactgttgacattccctcctccaacctcaggttctttctctcaagaggaagcccttttataactgcttctgtcacatcttcaacatctctttccatcacaacactgcacaccatactctctttgtctcccagtaatgacaaaaacaccaagtacacccttaagctcaacaacactcagacatggctcatatacgcctcctccccaatctatttgaatcgtgatggtgcttcccaggttacatcgaaaccattctccggcatcattcgtgtagcagcgttgcctgatgacaaccccaacaatgtcgcaattctcgacaagttcagctctagctacccttcatcgggtaatgcaacgctacacgatcctttccgtttggtgtatcaatggcagaaggaaggttctggggatttgctcatgctggctcaccctcttcatgctaaacttttatcacataataacaccggtaatgtcaatattttgcgtgattttaagtatagaagcattgatggtgaccttgttggtgttgttggagattcatggaagttggaaatgaatcctattcccgtgacatggcattctaacaaaggcgtgggaaaagagtcatacaacgaaattgtctcagcactttccaaggatgttcaaaccctaaactctccaatatcaacgccatcctcctacgcaattgggaaacttattggaagggctgcaaggttggcgttgatagcggaagaagtgtcttttccaaacgtggttcccaccatcaaggagtttctgaagcggaatattcagccatggttggatggaacagtccaagggaatggctttctatatgaaaaaaaatggggtggacttgtaacgaaaatggggtcaactgattcaagcgctgattttgggttcggagtgtacaacgatcaccattaccatttggggtactttctttacggaattgcggttctagcaaagatagacaacgagtggggacaaaaatacaagccacaagtttatgcacttttgtcagatttcatgaacttggagcaacaaaacgctcattatccacgtctaaggtgttttgacctctacaggttacactcttgggcttcaggggtgacagaatttgcagatggaaggaaccaagaaagtacaagtgaagctgtgaatgcatactattcagcagctttggtgggtgtagcatacggagacaaaagtcttgttagcgccggatcaacgctattggcgatggaaattcttggtacacaaacatggtggcatgtgaaagcagaagacaagttgtacaatgaagagtttgcaaaaaacaataagatagttggtgttctgtggtctaacaagagggacagtggattatggtgggcccctgctacatgcagagagtgcaggcttggaatccaagtgctacccttgtcgccgatcactgagacattgttctctgatgctggttatgtgaaggggcttgtggaatggacattgccctctttaagtagtgaggcttggaagggaatgacctatgcattgcagggagtttatgataagcaaacagcattgcagaacataagaaggttgaaaggttttgatgatgggaactcattcactaatctcttgtggtggattcacagcagataa

SEQ ID NO: 23 - DNA, 1974 bp, Vigna unguiculata
  source 1..1974: mol_type = genomic DNA
atgtcttcttcatcttcttttctgttccctcaaactcaatccacagttctcccagacccttcaacctacttctcctccaaccttctttcatctccactccccacaaactctttcttccaaaactatgttatcccaaacgggtcacaacctgagtacattcacccttacctcatcacaacttcaaactcctctctatcagcctcatacccttttctcctcttcaccacagcactcttgtaccaagcttttgtgccggatctcaccatctcttccactcaaacacagtcacacgatcaacgaaaccgtgtaatctcatcattcagtgaccttggcatcactttggacattccctcctccaacctcagcttctttctctcaagaggaagcccttttataactgcttccgtcacatcttcaacatctctttctatcacaacactgcacaccatactctctttgtctcccagtaatgacaacaacaccaagtacacccttaagcttaacaacacccagacatggctcatatacgcctccgacccaatctatctgaaccgtgacggtgcttccgaggttacatcgaagccattttctggcatcattcgtgtagcagtgttgcctgatcctaactatgcgacaactcttgacaagttcagctcttgttaccctttgtcgggtgatgcaacactgaaggagtctttccgtttggtgtatcaatgggaaaaggaaggttctggggatttgctaatgctggctcaccctcttcatgttaaacttttatcaaataaaagtaacgggcaggttactgtgctgagtgattttaagtatagaagcattgatggcgatcttgttggtgttgttggagattcatgggttctggaaacggatcgtattcctgtgacatggtattctagcaaaggagtggaaaaagattcgtacgatgagattgtgtcagcgcttgttaaggatgtggagaagcttaactcttccgcaataggaacaagttcatcttatttttatggaaagcgagttgggagagctgcaagattggcactgatagcggaagaagtgtctttttcaaaggttgttcccaccattatggattttcttaaagaagccatcgagccttggttagatggaactttcgtagggaatggttttctatatgaaaacaaatggagtggacttgtaaccaaactagggtcaacggattcaaccgctgatttcgggtttggagtttacaatgatcaccattatcatttggggtactttctatatggaattgcggttcttgcaaagattgatcccgagtggggacaaaaatacaagccacaagtatattcactagtgacagattttatgaacttgggtcaaaggtatagcagaatttatccacgtctaaggtgttttgacctttacatgttacattcttgggccgcaggagtgactgaatttgaagatggtaggaaccaagaaagtacaagtgaagctgtgaatgcgtactattcagcagcgttggtgggtctggcatatggtgattcaaatcttgttgaaactgggtcaacgttagtggcgttggaaattctagctgcacaaacttggtggcatgttaaagtggaagacaacttgtacaatgaagaatttgcaaaagacaataggatagtgggaattttgtgggctaataagagggatagtaagttatggtgggcgagtgctgaatgtagagaatgcagactcggaatccaagtgctacccttgttgcctatcactgagacattgttctctgatgctgattatgtgaaggagcttgtggaatggacattgccatctttaagtagtgaggggtggaagggaatgacctacgcattgcagggaatttatgataaggaaacagcgttgcagaacataagaacgttgacaggttttgatgatggaaactcttacagtaatctcttgtggtggattcacagcagatga

SEQ ID NO: 24 - DNA, 1971 bp, Vigna unguiculata
  source 1..1971: mol_type = genomic DNA
atgtctccttcttttctattccctcaaactcaatccacagttctcccagacccttcaacctacttctcaccaaaccttctttcttctccattccccacaaactctttcttccagaacttcgttattccaaatggtacacagcctgagtattttcacccctatcacattcaggcctcaaactcctcactctctgcctcctacccttttctcttcttcacagcagcagtgttgtaccaagtttttgtcccagatctcaccatttcagcctctcaaacaacctcctatggacaaaaccgtgttatctcatcctacagtgacctcggtgtcactttggacatcccaagttccaacctcaggttctttcttgtcagaggaagccctttcataactgcttctgtcacaaaaccaacctctctttccatcaaaacagtgcacaccatactctctttgtcttcctatgatggcaataccaagtttatcattcagcttaacaacactcagacatggctcatatacacctcgtcccctatctatttgaaccatgttccttccgaggttacatccaagccgttttctggcatcattcgtatagcagcgttgcctgattccaaccccagtaatgtcgcaactcttgacaagttcagttcttgttaccccgtgtcgggtgatgcaacactcggcaagcctttccgtttggagtataaatggcaaaagaaaaggtcaggggacttgctcatgctagctcaccctcttcatgctaagcttctatcacgtgactgtaacgttaccgttctgcacgattttaagtatcgaagtgttgacggtgatcttgttggtgttgttggagattcgtgggtgttggagacggatcctattcctgtcacatggcattctaagaaagggatcagtaaagagtcgtttggtgagattgtttctgcactttataaggatgtcaaggggctgaattcttctgcaataacaacaaattcatcttatttctatgggaagcttgttggaagggctgcaaggttagccttgatcgcagaagaagtgtcttattacaaggtgattcccaagattagaaagtttctgaaggaaaccattgagccctggttggatggaactttcaaagggaatggttttctatacgaaagaaaatggcgtggacttgttactgaacaaggctccacagattcaactgctgattttggttttggaatatataacgatcaccattttcatttggggtacttcctttatggaattgcagttcttgcaaagattgaccctgcctggggcaaaaaattcaaaccgcaagcttattcacttgcgacagattttatgaacttgggccaaagatataactcagattatccacgcctaaggtgttttgacctttacaagttacactcttgggcttcagggctgactgaatttgaagatggaaggaatcaggagagtacaagcgaagctgtaaatgcatactatgcagcagctttgatgggtctggcttatggtgatagccgtcttgttgatactggatcgacactgttagcattggaaattcgtgctacacaaacatggtggcatgtaaaagcagaagacaacttgtatgaagaagaatttgcaaaagataacaggatcgtgggtattctgtgggctaacaagagggacagtaagctatggtgggctactgcggaatgtagagagtgcaggcttagtatccaagttctacccttgttacctgtcacagagaccttgttctctgatactgtttatacgaaggagcttgtggaatggacactaccttctttgaagaataaaacgaatgtagaaggctggaagggattcacctatgccttgcaaggaatttatgataaaagtacagcattaaagcaaataagaaggttgacaggttttgatgatggaaactcattcagtaacctcctctggtggattcacagcagatga

SEQ ID NO: 25 - DNA, 1968 bp, Phaseolus vulgaris
  source 1..1968: mol_type = genomic DNA
atgtcttcttcatcttcttttctcttccctcaaactcaatccacagttcttccagacccttcaacctacttctcctctaaccttctttcatctccacttcccacaaactctttcttccaaaactatgttatcccaaacgggtcacaacctgagtacattcacccttacctcatcaaaagtacaaactcctcactatcagcctcataccctcttctcctcttcaccacagcactcttgtaccaagcttttgtgccagatctcaccatctcttccactcaaacacactcacagcaacaaaaccgtgtaatctcatcattcagtgaccttggtgtcactttggatattccctcctccaacctcaggttctttctctcaagaggaagcccttttataactgcttccgtcacttcttcaacatctctttctatcacaacactgtacaccatactctctttgtcttccaacaatgagaacaacaccaagtacacccttaagcttaacaacactcagacatggctcatatacacctcctcccccatccatttcaaccataatgcttcagaggctacgtccaagccattttctggcatcattcgtgtagcagtgctgccaaatcctaactatgagacgattcttgacaagtacagctcttgttaccctttgttgggtgatgcaacactagaggagccttcccgtgtggtgtatcaatggcaaaaggaagggtctggggatttgctcatgctggctcaccctcttcatgttaagcttttatcaaataataataacgggaatgttactttgctgagtgattttaagtacagaagcattgatggtgatcttgttggtgttgttggagattcatggatattgcaaacggatcgtattcctgtgacatggtattctaacaacggagtggaaacaaattcatatgatgagattgtctcagcgcttgttaaggacgtgcaagcgcttaattcttcagcaataggaacaacttcatcttatttttatggaaagcgcgttggaagggccgcaaggttggcattgatagcggaagaagtgtcgttttcaaaggttgttcccacggttacggattttcttaaagaggccattgagccttggttagatggaactttcgaagggaacggttttctatatgaaaataaatggggtggacttgtaaccaaactggggtcaacggattcaagcgctgattttgggtttggagtttacaatgatcaccattaccatttggggtactttctatatggaattgcggttcttgcaaagattgatcccgagtggggacaaaaatacaagccacaagtttattcacttgtgacagattttatgaacttgggtcaaaggtataacagaaattatccacgtctaaggtgttttgacctttatacgttacattcttgggctgcgggagtgactgaatttgaagatggtaggaatcaagaaagcacgagtgaagctgtgaatgcatactattcagcagcgttggtgggtctggcatatggtgactcgagtcttgttgccactgggtcaacgttggtggcgttggaaattctagctgcacaaacttggtggcatgtgaaagtggaagacaacttgtacgaagaagaatttgcaaaagacaatagaatagtggggattgtgtgggctaataagagggatagtaagttatggtgggccggtgcagactgtagagaatgcagacttggaatccaagtgctacccttgttgcctatcactgagacactgttctctgatgctgattatgtgaaggagcttgtggaatggacatttccctctttaagtagtgaggggtggaagggaatgacctatgccttgcaaggagtttatgataagcaaacagcactgcagaatataagaacattgaaaggttttgatgatggaaactcttacagtaatctcttgtggtggattcacagcagataa

SEQ ID NO: 26 - DNA, 1968 bp, Phaseolus vulgaris
  source 1..1968: mol_type = genomic DNA
atgtctttctcatcttcttttctcttccctaaaactcaatccacagttctcccagacccttcaacctacttctcttcaaaccttgtttcttctcctctccccacaaactcttttttccaaaactttgtccttttaaacgggtcacaacctgagtacattcacccctacctcatccaaacctcaaagtcctcactctctgcctcataccctcttctcttcttcactgcagcagtgttgtaccaaacttttgtgccggatctcacaatctcttccactcaaacacttccaaatgaacagaaccatgtaatctcatcccacagtgaccttggtgtcactttggacattccctcctccaacctcaggttctttctctcaagaggatgcccttttataactgcttccgtcacatcttcaacatctctttctatcagaacactgcacaccatactctctttgtcttccaacaatgagaacaacaccaagtacacccttaagcttaacaacactcagacatggctcatatacacctcctcccccatccatttcaaccataatgctttagaggttacgtccaagccattttctggcatcattcgtgtagcagtgctgccaaatcctaactatgagacaattcttgacaagtacagctcttgttaccctttgttgggtgatgcaacactagaggagccttcccgtgtggtgtatcagtggcaaaaggaagggtctggggatttgctcatgctggctcaccctcttcacgttaagcttttatcaaataataatactggtactgacactattttgcataattttaagtatagtagcattgatggtgatcttgttggcgttgttggagattcatggaagttggaaatgaatcatattcctgtaacatggcattctaacaaaggagtggaaaaagagtcatatgatgaaattgtctcagcactttccaaggacgttcaagcactaaactcttcaccaatagcaacagcatcctcctatttatatgggaaacttattggaagggctgcaaggttggcgttgatagcggaagaagtgtcttttccaaacgtggttccaacgattaaggagtttctgaagaagaatattgagccttggttggatggaacattccaagggaacggttttctatatgaaaataaatggggtggacttgtaacaaaactggggtcaacagattcaagcgctgattttgggtttggagtgtacaatgatcaccattaccatttgggttactttctttatggaattgcggttctagcaaagattgaccctgagtggggacaaaaatacaagccacaagtttattcacttttgtcagattttatgaatttggaccaccaacacaacgcttattatccacgtctaaggtgttttgacctctacatgttacattcttgggcttcagggttgaaagaatttgcagatggacggaaccaagaaagtacaagtgaagctgtgaatgcgtactattcagcagctttggtgggtctagcatatggtgactcaagtcttgttgccactgggtcaacgttagtggcgttggaaattcttgctgcacaaacttggtggcatgtgaaagtgggagagaagttgtacaaagaagagtttgcaaaagacaataggatagttggtgttctgtgggctaataagagagatagtggactatggtgggcgagtgcagagtgtagagaatgcagacttggaatccaagtgctacccttgttgcctatcactgagacattgttctctgatgctgattatgtgaaggagcttgtggaatggacacttccctctttaagtagtgaggggtggaagggaatgacctatgccttgcaaggagtttatgataagcaaacagcattgcagaatataagaacgttgaaaggttttgatgatggaaactcttacactaatctcttgtggtggattcacagcagataa

SEQ ID NO: 27 - DNA, 2004 bp, Phaseolus vulgaris
  source 1..2004: mol_type = genomic DNA
atgtcacaattccccttctcttcctcacaaacaatgtcttcttcttttctcttccctcaaactcaatccacagtcctcccagacccttcaacctacttctcaccaaacctcctttcatctccactccccacaaactctttcttccaaaactttgttattccaaatggcacagtgcctgagtactttcatccctaccacattcagtcctcaaactcctcactctctgcctcctacccttttctcttctttacagcagctgtgttgtaccaagtttttgtcccagatctcaccatctctgcctctcagacatactcaaatgcacaaaaccgtgtaatctcttcctacagtgaccttggtgtcactttggacattcccacttccaacctcaggttctttcttgtcagaggaagccctttcataactgcttctgtcacaaagccaacctctctttccatcacaaccgtgcacaccatactttctttgtcttcctatgatgacaacaccaagtttatccttcagcttaacaacactcagacatggctcatatacacctcctccccaatctatttgaaccatgctgcttctcaggtttcatccaagccattttctggcatcatccgtatagcagctttgcctgattccaaccccaacaatgtcgcaactcttgacaagttcagttcttgttaccctgtgtcgggtgatgcagcactcaagaaacctttccgtgtggagtataaatggcaaaggaaaaggtcaggggacttgctcatgctagctcaccctcttcatgctaagcttctatcacacgattgtaacgttaccgttctgcacgattttaagtatagaagtgttgacggtgatcttgttggtgttgttggagattcatgggtgttggaaacggatcctattcctgtcacatggcattctaaaaaaggcatcgataaagagtcatttggtgagattatctcagcgcttaataaggatgtgaaggagctaaattcttctgcaataacaacacagtcatcttatttctatgggaagcttgttggaagggctgcaaggttggccttgatcgcagaagaagtgtcttatcctaaagtgattcccaagattagaaattttttgaaggaaaccattgagccctggttggatggaactttcaaagggaatgcttttctatatgaaagaaaatggcgtggacttgttactaaacaaggctccacggattcaactgctgattttgggtttggagtgtataacgatcaccattttcatttggggtactttatttatggaatcgcagttcttgcaaagattgaccctgcctggggcaaacaatacaaaccgcaagcctattcacttgtgacagattttatgaacttgggccaaagatataacacagattatccgcgcctaaggtgttttgacctttacaagttacactcttgggcttcagggctgactgaatttgaagatggaaggaatcaggagagtacaagtgaagctgtaaatgcctactatgcagcagcattgatgggtctagcttatggtgatagccgtcttgttgatactggatcaacactgttagcattggaaattcgtgctacacaaacatggtggcatgtaaaagtggaagacaacttgtatgaagaagaatttgcaaaagataacaggatagtgggtattctgtgggctaacaagagggacagtaagctatggtgggctcctgcagagtgcagagagtgtaggcttagtatccaagttctacccttgttgcctgtcactgagaccttgttctttgatactgtttatgccaaggagcttgtggaatggacactgccttctttgaagaacaaaacaaatgtagaaggctggaagggattcacctatgccttgcaaggaatttatgataaaactacagcattaaagaaaataagaatgttgacaggttttgatgatggaaactcattcagtaatctcctgtggtggattcacagcagataa

SEQ ID NO: 28 - DNA, 1959 bp, Glycine max
  source 1..1959: mol_type = genomic DNA
atgtcttcttttcttttccctcaaacacaatccacagtcctctcagacccttcaacctatttctcctcaaacctcctttcatctccactccccacaaactctttcttccaaaacttcgttattccaaacgggtcccaagctgagtacattcacccttacctcatcaaaacctcaaactcttcactatcagcttcataccctcttctgatcctcttcaccactgcagtgttgtaccagacttttgtggcagatatcaccatctcttcaactcaaacaacctcacaaaaccatgtaatctcatcatacagtgaccttggtgtcactttggacattccctcctccaacctaaggttctttctctcaaggggaagcccttttctaaccgtttctgtgacatctccaacatctctttccatcacaacagtgcataccatagtctctttgtcttccaatgatgacaacaacaccaaatacacccttaagcttaacaacactcaaacatggctcatatacacttcctcaccaatctatttcacccataataatgcttcagaggttacatccaagccattttctggcatcattcgtgtggcagtgttgcctaaccacaactacgtaacaattcttgacaagttcagcacttgttaccctttgtcgggtaatgcaacactcgtagagcctttccgtgtggtgtatgaatggcaaaaggaaggttctggggacttgctcatgctagctcaccctcttcatgttaagcttctatcaaataattataatggtctagttactgtgctgaacgattttaagtatagaagcattgatggtgatcttgttggtgttgttggagactcatgggtgttggaaaccaatcctattcctgtgacatggtattccaacaaaggtatggaaaaagattcttatgatgagattgtctcggcacttgttaaggatgtgcaagagctgaattcttcatcaataggaacaagttcatcttatttttatggaaagcgtgttggaagggctgcaaggttggcgttgatagcggaagaagtttctttttctaacgtggttcccacgattaagaagtttcttaaggagtctattgagccttggttggatggaactttacaagggaatggatttctatacgaaaataaatggggtggacttgtcaccaaactggggtcaacggattcaacagctgattttgggtttggagtgtacaatgatcaccattatcatttgggatacttcctttatggaattgcggtccttgcaaagattgatcctgagtggggacaaaaatacaatccacaagtttattcacttgtcacagattttatgaacttgggccaaaaatataactctcgttatccacgtctaaggtgttttgacctttacaacttacactcttgggcttcaggagtgactgaattcgcagatggaaggaatcaagaaagtacaagtgaggctgtgaatgcgtactattcagcggcattggtaggtttagcatatggtgactcaaatcttgttgccattggatcaacactactggctttggaaattcttgctgcacaaacttggtggcacgtgaaagcagaaggcaacttgtacgaagaagaatttgcaaaagagaacaaaatagtgggtgttctgtgggctaacaagagagatagtgccctatggtggggccctgctacgtgtagagagtgtaggcttggaattcaagtgctaccattgtctcctgttactgagactttgttctctgatgctgattatgtgaaggagcttgtggaatggacaatgccctctttgactagtgaagggtggaagggaatgacctatgccttgcaaggaatttatgataaggaaacagcattggaaaatattagaaagttgaaaggttttgatgatgggaactcgttgagtaatctcttgtggtggattcacagcagatga

SEQ ID NO: 29 - DNA, 1896 bp, Glycine max
  source 1..1896: mol_type = genomic DNA
ctcccaaacccttcaacctacttctcctcaaaccttgtttcatctccactccccacaaactctttcttccaaaactttgctcttcaaaatgggacacaagctgagtacattcacccttacctcatcaaaacctcaaacttttcactctcagcctcatgccctcttctcctcttcaccacagcagtgttgtaccagacttttgtggcagatatcactatctcttcaactcaaacaacctcacaaaaccatgtaatctcatcatacagtgaccttggcgtcactttggacattccctcttccaacctaagaggaagcccttatataaccgcttccgtaacaaagccaacatctctctccatcacaacagtgcgctccatagtttctttgtgttccaataataaggaaaacaccaagtacacccttaagcttaacaacactcagacatggctcatatacacctcctccccaatatatttgaaccatgatgctgcttccaacattacttccaagcattttctgcagtgttgcctgtttccaacttcaagagtgtggcaattctcgacaagttcagctcttgttacccggtctttatcgggtaatgcaacactcgtgaagccttttcgtgtgacgtatgagtggcaaaagaaagggcctgggttcttgctcacgctagctcaccctcttcatgtcaagcttctacaatataaaaagaatcatcgcatgattgttctgcgtgattttaagtatagaagcattgatggtgatcttgttggtgttgttggagattcatggctgttaaaaactgataccattcctgtgacatggcattctaacaaaggtgtggaaaaagagtcacatgatgagattgtctcagcgctttctaaggatgttgaagcgctaagttcttcaccaatagcaacagaatcgtcttattattatgggaaacttattggaagggctgcaaggttggcgttgatagcagaagaagtgtcttctcctaatgtgattccaacgattcagaagtttctgaaggatagtattgagccttggttggatggaactttccaagggaatggttttctatatgaaaacaaatggggtggacttgtcaccaaacaagggtcaacagattcaggagctgattttgggtttggagtgtacaatgatcaccattatcatttggggtactttctttatggaattgcggttcttgcaaaggttgaccttcaatggggacaaaagtacaagccacaagtttattcacttgtgtcagattttatgaactcgggccaaaaatataactcacattatccacgtctaaggtgttttgacctttacaagttacactcttggacttcaggggtgactgaatttacagatggacggaatcaagaaagtacaagtgaggctgtgaatgcgtactattcagcagcattggtaggtttagcatatgatgactcaaatcttgttgccactgggtcaacactactagctttggaaattcttgctgcacaaacttggtggcatgtgaaagcagaaggcaacttgtacgaagaagaatttgcaaaagagaacaaaatagtggatgctctgtgggctaacaagagagatagtgcactatggtgggcccctgctacgtgtagagagtgtaggcttggaatcgaagtgctaccattgtctcctgttactgagacattgttctatgatgctgattatgtgaaggagcttgtggaatggacaatgccctctttgactagtgaaggatggaagggaatgacatatgccttgcaaggaatttatgataaggaaacagttttgcagaatattagaatgttgacaggttttgatgatgggaattcattcactaatctcttgtggtggattcacagcagatga

SEQ ID NO: 30 - DNA, 1962 bp, Glycine max
  source 1..1962: mol_type = genomic DNA
atgtcttctaattttctcttccctcaaactcaatccacagtcctcccaaacccttcaacctacttctcctcaaaccttctttcttctccactccccactaactctttctttcaaaactttgttattccaaacgggtcccaagctgagtacattcacccttacctcgtcaaaacctcaaactcttcactctcagcctcataccctcttctcctcttcaccacagcacttttgtaccaatcttttgtgccagatatcacaatctcttccactcaaacacactcaaatcaacaaaaccgtgaaatctcatcatacagtgacctcagtgtcactttggacattccctcctccaacctaaggttctttctctcaagaggaagcccttttataaccgcttctgtgacatctccaacatctctttcgatcacaacagtccacaccatagtctctttgtcttccaatgacgacaacaacaccaagtacacccttaagcttaacaacactcaaatatggctcatatacacctcctccccaatctatttgaatcatgatggcgcttccaatattacatccaagccattttctggcataattcgtgtagcagcgctgcctgattccaactccaagagtgtagcaattctcgacaagttcagctcttgttaccctttgtcgggaaatgcaacactcgtggaacctttccgtgtggtgtatcaatggcaaaaggaaagttctggggacttgctcatgctagctcaccctcttcatgttaagcttttatcaaatagtcaagttactgtgctgaaagattttaagtatagaagcattgatggcgatcttgttggtgttgttggagattcatgggtgttggaaacggatcctattcctgtgacatggtattctaacaaaggtgtggataaagattcgtatgatgaggttgtctcggcacttgttaaggatgtgcaagagctaaattcttcagcaataggaaccagttcatcatatttttatgggaagcgtgttggaagggctgcaaggttggcgttgatagcggaagaagtgtctttttccaacgtggttcccacgattaagaagtttctgaaggagtctattgagccttggttggatggaacttttcaagggaatagttttctatatgaaaataaatggggtggacttgtcaccaaacaaggctctacagattcaactgctgactttgggtttggagtgtacaatgatcaccattatcatttagggtactttctttatggaattgcggttcttgcaaagattgatcctcagtggggacaaaaatacaagccacaagtttattcacttgtcacagatttcatgaacttgggccagagatataacagattttatccacgtctaaggtgttttgatctttacaaattgcactcttgggctgcagggttgaccgagtttgaggatggaaggaatcaagaaagtacaagtgaggctgtgaatgcatactattcagcagcattggtgggtcttgcatatggtgactcaagtcttgttgacactgggtcaacgctagtggcattggaaattctagccgcacaaacttggtggcatgtgaaagtggaagacaacttgtatgaagaggaatttgctaaagataacaagatagtgggggttttgtgggctaacaagagggatagtaagctatggtgggccagtgctgagtgtagggagtgtaggcttggcatccaagtgctacccttattgcctattactgagacattgttctctgatgctgattatgtgaaggagctagtggaatggacagtgccctttttaagtagtcaagggtggaaggggatgacctatgccctgcaaggaatttatgataaagaaacagcattggaaaatataagaaagttgaaaggttttgatgatgggaactctttgagtaatctcttgtggtggattcacagcagatga

SEQ ID NO: 31 - DNA, 1974 bp, Glycine max
  source 1..1974: mol_type = genomic DNA
atgtcttcttcttttctcttccctcaaactcaatccacagtcctcccagacccttcaacctacttctcaccaaaccttctttcttctccactccccacaaactctttcttccaaaactttgttattccaaatgggacacagcctgagtacattcacccctaccttatcaaaacctcaaactcctcactctcagcctcataccctcttctctttttcaccacagcagtgttataccaagcttttgtgccagatatcactatctcttcccctcaaacacactcacgtcaacaaaaccgtgtaatctcatcatacagtgaccttggtgtcactttggacattccctcttcaaacctaaggttctttctctcaagaggaagcccttttataactgcttctgtgacaaagccaacatctctctccatcacaacagtgcacaccattgtctctttgtctgccaatgatgacaaaaacactaagtacacccttaagcttaacaacactcaggcatggctcatatacacctcctccccaatctatttgaaccatgatgctgcttccaacgttacatccaagccattttctggcataattcgtgtagcagtgttgcctgattccaactccaagtgtgtaaaaattctcgacaagttcagctcttgttatcctttgtcgggtaatgcaacactcgagaagcctttccgtgtggtgtatgaatggctaaaggaaggttctgggaacttgctaatgctagctcaccctcttcatgtcaagattttatcatctactaataatggtcaagttaatgtgcttcgtcattttaagtatagaagcattgatggtgatcttgttggtgttgttggagactcatgggtgatggaaaccaatcctattcctgtgacatggtattctaacaaaggtgtggagaaagagtcatatgatgaaattgtctcagcgcttgttacggatgtgcaagggctgaattcttcagcaatagaaacaataatttcatcttatttttatgggaagcgtgttggaagggctgcaaggtttgcgttgatagcagaagaagtgtcttttcccaaggtgattccttcagttaagaagtttctgaaagagactattgagccttggttggatggaactttcccagggaatggttttcaatatgaaaataaatggggtggacttgtaaccaaactagggtcaacggattcaaccgctgattttggttttggaatttacaatgatcaccattaccatttggggaacttcctttatggaattgcggttcttgcaaagattgaccctcaatggggacaaaagtacaagccacaagtttattcacttgtgacagatttcatgaacttggggccaagttataacagattttatccacgtctaaggaattttgacctttacaaattgcactcttgggctgcagggttgactgaatttgaacatggaaggaatcaggaaagcacaagtgaggctgtgactgcttactattctgcagcgttggtgggtcttgcatatggtgactcaagtcttgttgccactgggtcaacgttaatggcgttggaaattcttgctgcacaaacttggtggcacgtgaaagagaaagacaacttgtacgaagaagaatttgcaaaagagaatagggtagtggggattttgtgggctaacaagagggatagtaagttatggtgggccagggctgagtgtagagagtgtaggcttggaatccaagtgctaccattgttgcctattactgagacattgttctctgatgctgattatgccaaggagcttgtggaatggacactgccttctgcacgtagagaagggtggaagggaatgacatatgccttgcaaggaatttatgataggaaaacagcattgcagaatataagaatgttaaaaggttttgatgatgggaattcattcactaatctcttgtggtggattcatagcagatga

SEQ ID NO: 32 - DNA, 1968 bp, Glycine max
  source 1..1968: mol_type = genomic DNA
atgtcttctccttcttcttttctattccctcaaactcaatccacagtcatcccagacccttcaacctacttctcacctaaccttctttcttctcctctccccacaaactctttcttccaaaactttgttattccaaatgggacacaacctgagtacattcacccttacctcatccaatcctcaaactcttcactctcagcttcataccctcttctcctcttcaccacagcactgttgtaccaagcgtttgtgccagatctcaccatctctgccactaaaagatactcatcataccaacaaaaccgtgtaatctcatcctacagtgaccttggtgtcactttggacattccaagctccaaccttaggttctttcttgtcagaggaagcccttatataactgcttctgtcacaaagccaacacctctttccatcaaaacagtgcacaccatagtttctctgtcttccgatgattccaacaccaagcacacccttaagcttaacaacactcagacatggatcatatacacgtcctccccaatctacttgaaccatgttccttctgaggttacatccaagccattttccggcatcattcgtatagcagcgttgcctgattctggttccaagtatgttgcaactcttgacaagttcagttcttcttaccctgtgtctggtgatgcagcactcaagaaaccattccgtctggagtataaatggcaaaagaaacgttctggggacttgctaatgctggctcaccctcttcatgtcaagcttctatcatatgatcgtgatgttactgtgctgaatgattttaagtacagaagcattgatggtgatcttgttggtgttgttggagactcatgggtgttggaaaccaatgctattcctgtgacatggtattctaacaaaggtgtggacaaagagtcttatggcgagattgtctcggcgcttgttaaggatgttcgagcgctgaattcttcagcaataggaacaaattcatcttatttctatgggaagcaggttggaagggcggcgaggttggtgttgatagcggaagaagtgtcgtatcctaaagtgattccaaaggttaagaagtttctgaaggagactattgagccttggttggatggaactttcaaagggaatggttttctctatgaaagaaaatggcgtggacttgttactaaacaaggctctacagattcgactgctgattttgggtttggaatttacaatgatcaccatttccatttggggtacttcatttatggaattgcagttcttgcaaagattgatcctcaatggggacaaaagtacaagccacaggtttattcacttgtgacagatttcatgaacttgggtcaaagatataactcggattacacacgcctaaggtgttttgatctttataagttacactcttgggctgcagggttgactgaatttgaagatggaaggaatcaggaaagtacaagtgaagcagtgaatgcatactatgcagcagcattgatgggtctagcatatggtgactcaagccttgttgccactggatcaacgctagtggcgttggagattcttgctgcacaaacttggtggcatgtgaaagcagaagacaacttgtatgaagaagaatttgcaaaagataacaggatagtggggattttgtgggctaacaagagggatagtaagctatggtgggccagtgctgagtgtagagagtgtaggcttggaatccaagtgctacccttgttgcctattactgagacattgttctctgatgctgattatgtgaaggagctagtggaatggacagtgccctttttaagtagtcaaggttggaagggaatgacctatgccttgcaaggaatttatgatagggaaacagcactgcagaatattagaaagttgacaggttttgatgatgggaattcgttcactaatctcttgtggtggattcacagcagatga

SEQ ID NO: 33 - DNA, 1962 bp, Glycine max
  source 1..1962: mol_type = genomic DNA
atgtcttcttcttttctcttccctcaaactcaatccacagtcatcccagacccttcaacctacttctcaccaaacctcctttcatcaccactccccacaaactctttcttccaaaactttgttattcctaatgggacacagcctgagtacattcatccttacctcatccaatcctcaaactcttcactctcagcctcattccctcttctcctcttcaccacagcactcctgtaccaagcttttgtgccagatctcactatctctgcctctaaaacatactcatcatatcaacaaaaccgtgtagtctcatcctacagtgaccttggtgtcactttggacattccaagctccaaccttcgattctttcttgtcagaggaagcccttatataactgcttctgtcacaaagccaacacctctttccatcaaaacagtgcacaccgtagtttccttgtcttcagatgattacaacaccaaacacacccttaagcttaacaacagtcaggcatggatcatatacacctcctctccaatctacttgaaccatgttccttctgaggttacatccaagccattttctggcatcattcgtatagcggctttgcctgattctgattccaagtatgtggaaactcttgacaagttcagttcttgttaccctgtgtctggtgatgcagcactcaagaaaccattcagtgttgagtataaatggcaaaagaaacgttctggggacttgctcatgctggctcaccctcttcatgctaagcttctgtcatatgatcgtgatgttactgtgctgaatgattttaagtatagaagcattgatggtgaccttgttggtgttgttggagattcatgggtgttggaaaccaatcctattcctgtgacatggaattctaacaaaggtgtggagaaagagtcttatggcgagattgtcacggcgcttgttaaggatgttcaagcgctgaattcttcagcaataggaacaaattcatcttatttctatgggaagcaggttggaagggctgcgaggttggcgttgatagcggaagaagtgtcttaccctaaagtgattccaaaggttaagaaatttctgaaggagactattgagccctggttggatggaactttcaaagggaatgcttttctctatgaaagaaaatggcgtggacttgttactaaacatggctctacagattcaactgctgattttgggtttggaatttacaatgatcaccatttccatttggggtacttcatttatggaattgcagttcttgcaaagattgatcctcaatggggacaaaagtacaagccacaagtttattcacttgtgacagattttatgaacttgggccaaagatataactcagattatacacgcctaaggtgttttgatctttataagttacactcttgggctgcagggttgactgaatttgaagatggaaggaatcaagaaagtacaagtgaagcagtgaatgcatactatgcagcagcattgctgggtctagcatatggtgactcaagtcttgttgacactggatcaacgctggtggcgttggagattcttgctgcacaaacctggtggcatgtgaaagcagaagacaacttgtatgaagaagaatttgcaaaagataacagaatagtgggtgttctgtgggctaacaagagggatagtaaactatggtgggcccctgctacgtgtagagagtgtaggcttggaatccaagtgctacccttgttgcctattactgagacattgttctctgatgctgattatgtgaaggagcttgtggaatggacagtgccctttttaagtagtcaagggtggaaggggatgacctatgccttgcaaggaatttatgataagaaaacagcattgcagaatattagaaagttgacaggttttgatgatgggaattcgttcactaatctcttgtggtggattcacagcagatga

SEQ ID NO: 34 - DNA, 1971 bp, Cajanus cajan
  source 1..1971: mol_type = genomic DNA
atgtctccttcttttctcttccctcaaacacaatccacagtcctccctgacccttcaacctacttctccccaaaccttctttcttctccactccccacaaactctttcttccaaaactttgttattccaaatgggtcacagcctgagtacattcacccttacctcatcaaatcctcaaacacctccctctctgcctcctacccatttctattcttcactgcagcaatattgtaccaggtttttgtgcctgatctcacaatctctgcctctcggacatattcaaataaacaaaaccgtgtagtttcatcctatagtgaccttggtgtcactttggacattccctcttccaacttaaggttctttcttgtcagaggaagccctttcataactgcttctgtgacaaagccaacatctctatccatcacaacaaatcaaaccattgtttctttgacttccactaatgacaacactaagcacacccttcagctcaacaacactcagacatggctcatatacacctcctcaccaatttatttgaatcatgttccttctgaggtcacatccaagccattttctggcataattcgtatagcagcgttgcctgattccaaccccaagaatgtggaaattcttgacaagttcagctcttgttaccccgtgtcgggtgatgcaacactcaagaaaccattccgtgtggtgtacaaatggcaaaagaagcagtctggggacttgctcatgctagctcaccctcttcatgctaagcttctatcatatgatcgtgaggttactgttctgcacgattttaagtatagaagtgtcgatggtgatcttattggtgttgttggagattcatgggtgttggaaacagatcctattcctgtaacatggcattctaacaaaggtatcaaaaaggagtcatatggtgagattgtctcagcgcttgttaaagatgtaaaggagctaaattcttctgcaataacaacaaattcatcttatttctatgggaagcttgttggaagggctgcaaggttggcattgatagcagaagaagtgtcttttccaaaagtgattcccaaggttaggaagtttctgaaggagactattgagccctggttggatggaactttcaaagggaatggttttctatatgaaagtaaatggcgtggacttgttactgaacaaggctctacggattcaactgctgattttgggtttggaatttataacgatcaccattttcatttggggtacttcctttatggaattgcagttcttgcaaagattgaccctgtctggggccaaaaatacaaatcacaagcttattcacttgtgacagattttatgaacttggaccaaagatataactcagattatccacgcctaaggaattttgacctttacaagttacactcttgggcatcaggggtgactgagtttgaagacggaaggaatcaggaaagtacaagtgaagctgtgaatgcatactatgcagcagggttgatgggtctagcttatcgtgataccgatcttgttgccactggatcaaccctcttagcattggaaattcgtgctgcacaaacatggtggcatgtaaaagttggagacaacttgtacgaagaagattttgcaaaagataacaggatagtcggtgttctgtgggctaacaagagggacagtaagctatggtgggctcctgctgagtgtagagaatgtaggcttagtatccaagttctacccttgttgcctgttactgagaccttgttctctgatgctgtctatgcgaaggagcttgtggagtggacactgccttctttgaagaataaaacaaatgtagaaggctggaagggatttacctatgccttgcaagggatttatgataaaaatacagcattgaagaagataagaatgttgaaaggttttgatgatggaaactcgttcagtaatctcctatggtggattcacagtagatga

SEQ ID NO: 35 - DNA, 1971 bp, Cajanus cajan
  source 1..1971: mol_type = genomic DNA
atgtcttctccttttgtcttccctgaaacacaatccacagttctccctgacccttcaacctacttctccccaaacctactttcttctccgctccccacaagctcttttttccaaaatttcgttattccaaacgggtcacaacctgagtacattcacccttatctcatcaaaacctcaaacacatcactttctgcctcataccctttactcatcttcactgcagcagtgttgtaccaagcttttgtgccagatctcactatctcttccactcaaacacaaacaaaagaacaaaaccgtgtagtttcatcccacagtgaccttggtgtcactttggacattccctcttccaacttaaggttctttctttcaagaggaagtcctttcataactgcttctgtgacatctccaacgtctctctccatcacaaccaatcacaccatagcctctttatcttccaatgataacaaaaccaagcacacccttaggctcaacaacactcaaacatggctcatatacacctcttccccaatcaatttgaaccatgatgatggtgcttccgaggttacatccaaaccattttatggtacaattcgtctagcagtgttgcctgattccaaatatgaggcaactctcgacaagttcagctctagctaccctttatccggtgatgcaacatttgagaattcgaagccttttcgtttggtgtatcaatggcaaaagaaagggtctgagaatcttctcatgttagctcaccctcttcatgttaagcttttatcaaagtacaacaatgctggtgtcactgtgcttcatgattttaagtatagaagcatcgatggtgatcttgttggtgttgttggggactcatgggtattggaaatggatcctattcctgtgacatggtattctaacaagggtgtgaatgatggttcacgtgatgagattgtgtcagcgcttgttaaggatgtggaagcgttgaactcttcagcaataacaacaaaatcgtcttatttctatgggaagcaagttggtagggctgcgaggttggcattgatagcggaagaagtgtctttttccaaagtggttcccacaattaagaagtttttgaaagagaccattgacccttggttggatggaactttcaaagggaatggttttctatatgaaaaaaaatggggtggactagtaaccaaactagggtcaactgattcaacagctgattttgggtttggtgtttacaatgatcaccactttcatttgggttactttctttatggaattgcggttctagcaaagattgaccctgagtgggggcaaaagtacaagccacaagcttattcacttgtgacagattttatgaacttagaccaaaagtatagcacaatttatccacgtctaaggtgttttgacctttacaagctacactcctgggcttcaggggtgaccgaatttgaagatggaaggaatcaagaaagtacaagtgaggccgtgaatgcgtactattcggcagcattggtgggtctagcatatgatgactcaagtcttgtggccacagggtcaacgctagtggcattggagattcttgctgcacaaacttggtggcatgtgaaagtgggagaaaacttgtaccaagaggaatttgcacaagataataggatagtgggcattttgtgggccaacaagagggatagtaagctttggtgggccactgctgagtgtagagagtgtaggcttgggatccaagtgttacccttgctgcctatcactgagaccttgttctctgatgctgtttatgttaaggagcttgtggaatggaccatgccctatttgagtaatgaagggtggaagggcatgacctatgccttgcaagggatttatgataaggaaacagcattggatgagataagaaagttgaaaggttttgatgatggcaactcttacactaatctcttgtggtggattcacagcagatga

SEQ ID NO: 36 - DNA, 1971 bp, Phaseolus lunatus
  source 1..1971: mol_type = genomic DNA
atgtcttcttcttttctattccctcaaactcaatccacagtcatcccagacccttcaacctacttctcaccaaacctcctttcatctccactccccacaaactctttcttccaaaactttgttattccaaatggtacactgcctgagtactttcacccctaccacattcagtcctcaaactcttcactctctgcctcctacccttttctcttctttacagcagctgtgttgtaccaagtttttgtcccagatctcaccatctctgcctctcaaacgtactcacatggacaaaaccgtgtaatttcatcctacagtgaccttggtgtcactttggacattcccacttccaacctcaggttctttcttgtcagaggaagccctttcataactgcttctgtgacaaagccaacctctctttccatcacaaccgtgcacaccattctttctttgtcttcctataatgacaataccaagtttatccttcagcttaacaacactcagacatggctcatatacacctcctccccaatctatttgaaccatgctgcttctgaggttacatccaagccattttctggcatcattcgtatagcagcgttgcctgattccgaccccaacaatgtcgcaactcttgacaagttcagttcttgttaccctgtgtcgggtgatgcagcactcaaaaaacctttccgtgtggagtataaatggcaaaggaaaaggtcaggggacttgctcatgctagctcaccctcttcatgccaagcttctatcccatgattgtaacgttaccgttctgcacgattttaagtatagaagtgttgacggtgatcttgttggtgttgttggagattcttgggtgttggaaacggatcctattcctgtgacatggcattctaaaaaaggcatcaataaagagtcatttggtgagattgtctcagcacttaataaggatgtcaaggagctaaattcttctgcaataacaacacagtcatcttatttctatgggaagcttgttggaagggctgcaaggttagccttgatcgcagaagaagtgtcttatcctaaagtgattcccaagattataaagtttttgaaggaaaccattgagccctggttggatggaactttcaaagggaatgcttttctgtatgaaagaaaatggcgtggacttgttactaaacaaggctccacggattcaactgctgattttgggtttggagtgtataacgatcaccattttcatttggggtacttcgtttatggaattgcagttcttgcaaagattgaccctgcctggggcaaaaaatacaaaccgcaagcctattcacttgtgacagattttatgaacttgggccaaagatataactcagattatccgcgcctaaggtgttttgacctttataagttacactcttgggcttcaggactgactgaatttgaagatggaaggaatcaggagagtacaagtgaagctgtaaatgcctactatgcagcagccttgatgggtctagcttatggtgatagccgtcttattgatactggatcgacactgttagcattggaaattcgtgctacacaaacatggtggcatgtaaaagcggaagacaacttgtatgaagaagaatttgcaaaggataacaggatagtgggtattctgtgggctaacaagagggacagtaagctatggtgggctcctgccgagtgtagagaatgtaggcttagtatccaagttctacccttgttgcctgtcactgagaccttgttctttgatactgtttatgcgaaggagcttgtggaatggacactgccttctttgaagaacaaaacaaatgtagaaggctggaagggattcacctatgccttgcaaggaatttatgataaaactacagcattaaagaaaataagaatgttgacaggttttgatgatggaaactcattcagtaatctcctatggtggattcacagcagataa

SEQ ID NO: 37 - DNA, 1968 bp, Phaseolus lunatus
  source 1..1968: mol_type = genomic DNA
atgtcttcttcatcttcttttctcttccctcaaactcaatccacagttcttccagacccttcaacctacttctcctctaaccttctttcatctccacttcccacaaattctttcttccaaaactatgttatcccaaacgggtcccaacctgagtacattcacccctacctcatcaaaactacaaactcctcactatcagcctcatacccttttctcctcttcaccacagcagtcttgtaccaagcttttgtgccagatctcaccatctcttccactcaaacacactcacatcaacaaaaccgtgtaatctcatcattcagtgaccttggtgtcattttggatattccctcctccaacctgaggttctttctctcaagaggaagcccttttataactgcttccgtcacatcttcaacatctctttctatcacaacactgcacaccatactctctttatcttccaatgatgacgacaacaccaggtacacccttaagcttaacaactctcagacatggctcatatacacctcctcccccatccatttgaaccataatgcttcagaggttacgtccaagccattttctggcatcattcgtgtagcagtgctgcctaatcctaactacgagacaattcttgacaagtacagctcttcttaccctttgttgggtgatgcaacactagaggagccttcccgtgtggtgtatcaatggcaaaaggaagggtctggggatttgctcatgctggctcaccctcttcatgttaagcttttatcaaataataatgacgggaatgttactttgctgagtgattttaagtatagaagcatcgatggtgatcttgttggtgttgttggagattcatggatattgcaaacggatcgtattcctgtgacatggtattctaacaacggagtggaaacaaattcatatgaggagattgtctcagcgcttgttaaggacgtgcaagcgcttaattcctcagcaataggaacaaattcatcttatttttatggaaagcgcgttggaagggccgcaaggttggcattgatagcggaagaagtgtctttttcaaaggttgttcccacggttacggattttcttaaaggggccattgagccttggttagatggaactttcgaagggaatggttttctatatgaaaataaatggggtggacttgtaaccaaattaggatcaacggattcaagcgctgattttgggtttggagtttacaatgatcaccattaccatttggggtactttctatatggaattgcggttcttgcaaagattgataccgagtggggacaaaaatataagccacaagtttattcacttgtgacagattttatgaacttgggtcaaaggtataacagaatttatccacgtctaaggtgttttgacctttatatgttacattcttgggctgcaggagtaactgaatttgaagatggtaggaatcaagaaagtacgagtgaagctgtgaatgcatactattcagctgcattggtgggtctggcatatggtgactcaagtcttgttgccactgggtcaacgttagtggcgctggaaattctagcagcacaaacttggtggcatgttaaagtggaagacaacttgtacgaagaagaatttgcaaaagacaataggatagtggggattgtgtgggctaataagagggatagcaacttatggtgggccggtgcagactgtagagaatgcagacttggaatccaagtgctacccttgttgcctatcactgagacattgttctctgattctgattatgtgaaggagcttgtggaatggacacttccctctttaagtagtgaggggtggaagggaatgacctatgccttgcaaggaatttatgataagcaaacagcattgcagaatataagaacgttgaaaggttttgacgatggaaactcttacagtaatctcttgtggtggattcacagcagataa

SEQ ID NO: 38 - DNA, 2196 bp, Phaseolus lunatus
  source 1..2196: mol_type = genomic DNA
atgttgaaaaaacttagacgcaaggttagcacagccctcagaagtggccttaagaatgggtccaaaccctataaaaacccatcaccaccacctttatcaccattatcacttccattaccacttccattagaaccagtaagaacaatgtctcatactacaaaacattctccttttctgtttccacatgctaattcctctgttgttcctgatccctccaatttcttctccccaaaccttctctcaaatccactccccactaactctttcttccaaaactttaccttgaaaaatggtgatcaacctgagtatattcacccttacctcatcaaatcctcaaacttttctctctccctttcatacccatctcgcttttttaactcctccttcacttaccaggtcttcaaccctgatctcaccatttcttcctctcaaaagccccacctttcccatttcaaccataccatctcttcccacaatgatctcagtgtcactttggacatcccttcttccaatttgaggtttttccttgttaggggtagtccctttttgaccctctctgtgactcaaccgacccctctttccatcaccactattcacgccattctatccttttcctccagtgattccctcacaaagcacacttttaacctcaacaatggccagacttggattttgtatgcgtcttcgccgattaggttgagtcatggactttctgagatcaattctgatgcgttttctggcataattaggattgccctgttgcctgattctgattcgaagcacgaggctgtgcttgacaggttcagttcctgttaccctgtgagcggtgaggctgtgtttgccaggccattttgtgtggattataagtgggagaagaaaggatggggtgatttgttgatgttggcacaccctctccatcttcagcttttggctgatggtggttgtggagatgttaatgttctgagtgattttaagtacgggagcattgatggggaccttgttggggttgttggtgattcgtggagtttgaaaactgatcctgtttctgtgacttggcactctattaggggtgtgagagaagaatcccgggatgaggttgtttcggcgcttgtgaatgatgttgaggggctgaattcatcttcaataacgacgaactcgtcgtatttttatgggaaactgattgcaagggctgcaaggttggctttgatagctgaagagatgtgctttcttgatgtgattcctaaggttaggaagtatttgaaggaaaccattgagccgtggctggaggggacttttaatgggaatggatttctgtatgataggaaatggggtggcattgttaccaaacaagggtccaatgatgctggtgctgattttgggtttggaatttacaatgatcaccattatcatttgggatacttcgtttatggaattgcagtgcttgctaagattgatcctgtgtggggtaggaagtataagcctcaagcctattctctcatggcagattttatgacattgagcagaagatcaaattcgaactacacaagactaaggtgttttgacctttataaattacactcatgggctggaggtttaactgagtttgcagatggaagaaatcaggagagtaccagtgaagctgttaatgcatactattctgctgccttgatgggtctggcatatggtgacacacaccttgttgccactggatcaacgctcacagcattggagattcatgcagctcaaatgtggtggcatgtgaaacagggagataatcactatggtgaagggtttgagaaggagaacaaggtagttggtgttctttgggctaacaagagggacagtggactatggtttgcgcctcctgagtggaaagaatgtcggcttgggattcaactcttaccgttactgccgatttctgaagtgttgttctccgatgttgattttgtgaaggatcttgtggagtggacattgcctgccttgaacagggaaggtgttggagaaggatggaaagggtttgtttatgcactgcagggaatatatgataatgaaggtgcattgcagagggtaagaagcttgaatggttttgatgatggaaacacattgactaatctattgtggtggattcacagcagaagtgatgaagaggaatttggtcatggaaaacactgctggtttggtcattactgccactag

SEQ ID NO: 39 - DNA, 2220 bp, Phaseolus lunatus
  source 1..2220: mol_type = genomic DNA
atgtttaagaaacttggaagaaaaattgaaagagaaatcacaaaacccttcaaaaataaaccacgaccaagaacatcatctccacctccaccaccacctcctcctccaccaccacctccttcatctacacctccaccgccacctccgccgccatctcctcctcctcttcctaagcaaccaaatgctccatttctcttccctcaagctcactccacaattctccctgacccttcaactttctttgctccaaaccttctcccttctccactccctacaaactctttcttccaaaactatgttcttcaaaatggagatacacctgaatacattcacccctacctcatcaaatcctcaaactcctccctctccctctcctacccttctctcaacttcaactcttctttcatagcacaggttttcaaccctgacatcaccatctcttccactgatagcaaaaccaccccaggcttacacgcgagccacgtgatctcttccttcagtgatctaagtgtcactttggacattccctcttcaaacctcaggttctttcttgtcaggggaagcccttttgtgacagcatcagttacatgtcccacaccactttccatcaccaccatgcatgccattctttcactctcatccaataactctctcaccaaacacaccttgcagctcaacaatggccaatcatggctcattaacacttcctcgcccatcagtttaaaccacagcctttctgagattacttctggtgaattttctggcataattaggatagcagtgttgcctgattctgaccctaagtatgaggtaatcctcaataggttcagctcttgttatcctgtctctggggatgcaacattcacaaatccgttctgtgtaaagtataaatgggaaaagaaagggtggggggaattgttaatgctggctcaccctcttcaccttcagcttttgaatgatggtgatagtggtgtgacagttctgcataatttaaaatttagaagtattgatggagagcttgttggtgttgttggagactcctggctgctgaaaaccgacccggtttcagttacttggcattccacaagaggtataaaagaagaattccatgaagagatttattcagtgctttctgaagatgtggaagctttgaatcccaagggaataacaacaacatcatgctatttttatgggaagattatagcaagagcagcaaggttagcattgatagctgaagaggtggcttttctcgatgccatgcctgtgattaggaagttcttgaaggagatcattgagccatggttagacggtactttcagtggaaatggttttctctatgaggggaaatggggagggattgttactaaacaagggtctaaagattcagaagcagattttgggtttggtgtttataatgaccaccattacaatttggggtacttcctttacggaattgcggtgcttgcaaagattgatccagcttggggaaggaagtacaagcctcaagcgtattcacttgtggcagatttcatgagcttggcaagaagatcagactctaactacacgcgtttgaggtgttttgatctgtataaattgcactcttgggccggagggttaactgaatttgcagatggaagaaatcaggagagcactagtgaagctgtgaatgcatattattctgcagcattgacgggtctagcatatggtgacactcaacttattgccactggatcaacacttgcagcattggaaattcatgcagctcaaatgtggtggcatttgggagagggaaataaactgtatgaggaagattttacaaaagacaacaaggtggtaagtgttctgtgggctaacaagagagatagtggactatggtttgctccttctcagtggagagaatgcaggcttggtattcatgttttaccactgtctcctattacggaggccttgttctcagatgttgattatgtgaaggaacttgtggagtggacagtgcccaatttgaacaggaagtgtgttggagaagggtggaaggggtttatctatgccttggaaggaacttatgataaagaaagtgcactacaaaaggtaagaagcttgaaagtctttgatgatgggaactcaatgtctaatctgttgtggtggattcatagcaggggtgatgtggaggaggaatttggtcaaggaaaacaatgctggtttggccattactgccactaa

SEQ ID NO: 40 - DNA, 1989 bp, Phaseolus lunatus
  source 1..1989: mol_type = genomic DNA
atggttaagcaaaacaaaactcatttcatcttcccagagacacaatccactgtgcttcctgatccctccaacttcttctcctcaacccttctctcaaaaccactccccaccaactctttcttccaaaactttgtcctaaaaaatggtgatcaacctgaatacattcatccttacctcatcaaatcctctaactcttccctctctctctcatacccttctcgccaagtcagttctgctgtcatataccaagtcttcaacgctgatctcactatctcatccaagcaaagttccagtgggaaacaccttatctcctcctatagtgatctcagtgtcactctggatatcccttcttccaatcttagcttcctccttgttaggggaagcccctttttgactgtttctgtcacccaaccaacccctctttccatcaccaccatccacaccattctctcattctcttcaaatgagactaacaccaagtacacctttcagttcaacaatggtcaaacatggatcctttatgcttcctcctccatcaagttgagccacactctttctgagatcacttctgatacattttctggcatagtccggatagccttgttgcctgattctgattcaaaacacgaggcggttcttgacaagtttagttcttgttaccccgtgtctggtgaagctatatttagagaacccttttgtgtggagtataagtgggagaagaaagggtcaggagatttgctactcttggctcaccctctccatgttcagcttttgtctaatggagacaatgatgtcactgttctggaagattttaagtatggaagcattgatggggatgttgttggtgttgttggggattcatgggttttgcaaacagatcccgtgtatgtaacatggcactcaaccaagggagtcaaagaagaatcccatgatgaaattgtttcagccctttcgaatgatgttgacggcctaaactcatcatcgatttcaacaacttcgtcatatttttatgggaagttgattgcaagggctgcaaggttggcattgattgctgaggagttgagctaccctgatgtgattccaaaggttaagaagtttttgaaggaaaccattgagccatggttggtgggaactttcaatgggaatggatttctacatgataagaaatggggtggcattattacccaacaagggtccaatgatggtggtggtgattttggatttggtatttacaatgatcatcactaccatttggggtacttcctttatgcaattgcagtgctcgttaagcttgatccagcctggggtaggaagtacaaggctcaagcctattccattgtgcaagacttcatgaacttggacactaaactaaactccaattacacacgtttgaggtgttttgacctttatgtgcttcactcttgggctggaggattaactgagttcagtgatggaaggaaccaagagagcacaagtgaggctgtgtgtgcatattactctgctgctttgatggggctggcctatggtgatgctcatcttgtttcccttggatcaacactaacagcattggaaattcttgggactaaaatgtggtggcatgtggaagaggaagggaaattgtatgaggaagagttcacaagagagaacaggatcatgggggttctgtggtctaacaagagagacactggactatggtttgctcctgcagagtggaaagagtgtaggcttggcattcagctcttaccattggtacctatttctgaagccattttctccaatgctgagtatgtgaagcagcttgtggagtggactttgcctgctttgaatagggatggtgttggtgaaggatggaagggatttgtatatgcccttgaaggcatttatgacaatgaaagtgcattgcagaagataagaaaccttacaggttttgatggtggaaactctctcagtaatctcttatggtggattcacagcataggaaatgaataa

SEQ ID NO: 41 - DNA, 1971 bp, Phaseolus acutifolius
  source 1..1971: mol_type = genomic DNA
atgtcttcttcttttctcttccctcaaactcaatccacagtcctcccagacccttcaacctacttctcaccaaacctcctttcatctccactccccacaaactctttcttccaaaactttgttattccaaatggtacagtgcctgagtactttcacccctaccacattcagtcctcaaactcctcactctctgcctcctacccttttctcttctttacagcagctgtgttgtaccaagtttttgtcccagatctcaccatctctgcctctcagacatactcaaatgcacaaaaccgtgtaatctcatcctacagtgaccttggtgtcactttggacattcccacttccaacctcaggttctttcttgtcagaggaagccctttcataactgcttctgtcacaaagccaacctctctttccatcacaaccgtgcacactatactttctttgtcttcctatgatgacaacaccaagtttatccttcagcttaacaacactcagacatggctcatatacacctcctccccaatctatttgaaccatgctgcttctcaggttacatccaagccattttctggcatcatccgtatagcagctttgcctgattccaaccccaacaatgtcgcaactcttgacaagttcagttcttgttaccctgtgtcgggtgatgcagcactcaagaaacctttccgtgtggagtataaatggcaaaggaaaaggtcaggggacttgctcatgctagctcaccctcttcatgctaagcttctagcacatgattgtaacgttaccgttctgcacgattttaagtatagaagtgttgacggtgatcttgttggtgttgttggagattcttgggtgttggaaacggatcctattcctgtcacatggcattctaaaaaaggcatcgataaagagtcatttggagagattgtctcagcacttaataaggatgtcaaggagctaaattcttctgcaataacaacacagtcatcttatttctatgggaagcttgttggaagggctgcaaggttggccttgatcgcagaagaagtgtcttatcctaaagtgattcccaagattacaaagtttttgaaggaaaccattgagccctggttggatggaactttcaaagggaatgcttttctatatgaaagaaaatggcgtggacttgttactaaacaaggctccacggattcaactgctgattttggatttggagtgtataacgatcaccattttcatttggggtactttatttatggaattgcagttcttgcaaagattgaccctgcctggggaaaacaatacaaaccgcaagcctattcacttgtgacagattttatgaacttgggccaaagatataactcagattatccgcgcctaaggtgttttgacctttataagttacactcttgggcttcagggctgactgaatttgaagatggaaggaatcaggagagtacaagtgaagctgtaaatgcctactatgcagcagcattgatgggtctagcttatggtgatagccgtcttgttgatactggatcgacactgttagcattggaaattcgtgctacacaaacatggtggcatgtaaaagtggaagacaacttgtatgaagaagaatttgcaaaagataacaggatagtgggtattctgtgggctaacaagagggacagtaagctatggtgggctcctgcagagtgcagagagtgtaggcttagtatccaagttctacccttgttgcctgtcactgagaccttgttttttgattctgtttatgccaaggagcttgtggaatggacactgccttctttgaagaacaaaacaaatgtagaaggctggaagggattcacctatgccttgcaaggaatttatgataaaactacagcattaaagaaaataagaatgttgacaggttttgatgatggaaactcattcagtaatctcctatggtggattcacagcagataa

SEQ ID NO: 42 - DNA, 1968 bp, Phaseolus acutifolius
  source 1..1968: mol_type = genomic DNA
atgtctttctcatcttcttttctcttccctaaaactcaatccatagttcttccagacccttcaacctacttctcttcaaaccttgtttcttctccactccccacaaactcttttttccaaaactttgtccttttaaacgggtcacaacctgagtacattcacccctacctcatccaaacctcaaagtcctcactctctgcctcataccctcttctcttcttcactgcagcagtgttgtaccaaacttttgtgccggatctcacaatctcttccactcaaacactttcaaatgaacagaaccatgtaatctcatcccacagtgaccttggtgtcactttggacattccctcctccaacctcaggttctttctctcaagaggaagcccttttataactgcttccgtcacatcttcaacatctctttctatcacaacactgcacaccatactctctttctcttccaacaatgagaacaacaccaagtacacccttaagcttaacaacactcagacatggctcatatacacctcctcccccatccatttcaaccataatgcttcagaggttacgtccaagccattttctggcatcattcgtgtagcagtgctgccaaatcctaactatgagacaattcttgacaagtacagctcttgttaccctttgttgggtgatgcaacactagaggagccttcccgtgtggtgtatcagtggcaaacggaaggttctggggatttgctcatgctggctcaccctcttcatgttaagcttttatcaaataataatactggtactgtcactattttgcatgattttaagtatagtagcattgatggtgatcttgttggcgttgttggagattcatggaagttggaaatgaatcatattcctgtaacatggcattctaacaaaggcgtggaaaaagagtcatatgatgaaattgtctcagcactttccaaggacgttcaagcactaaactctacaccaatagcaacagcatcctcctatttatatgggaaacttattggaagggctgcaaggttggcgttgattgcggaagaagtgtcttttccaaacgtggttccaacgattaaggagtttctgaaggagaatattgagccttggttggatggaacattccaagggaacggttttctatatgaaaataaatggggtggacttgtaacaaaactggggtcaacagattcaagcgctgattttgggtttggagtgtacaatgatcaccattaccatttgggttactttctttatggaattgcggttctagcaaagattgaccttgagtggggacaaaaatacaagccacaagtttattcacttttgtcagattttatgaatttggaccaccaacataacgcttattatccacgtctaaggtgttttgacctctacatgttacattcttgggcttcagggttgaaagaatttgcagatggacggaaccaagaaagtacaagtgaagctgtgaatgcgtactattcagcagctttggtgggtctagcatatggtgactcaagtcttgttgccactgggtcaacgttagtggcgttggaaattcttgctgcacaaacttggtggcatgtgaaagtgggagagaagttgtacaaagaagattttgcaaaagacaataggatagttggtgttctgtgggctaataagagagatagtggactatggtgggcgagtgcagagtgtagagaatgcagacttggaatccaagtgctacccttgttgcctatcactgagacattgttctctgatgctgattatgtgaaagagcttgtggaatggacacttccctctttaagtagtgaggggtggaagggaatgacctatgccttgcaaggagtttatgataagcaaacagcattgcagaatataagaacattgaaaggttttgatgatggaaactcttacagtaatctcttgtggtggattcacagcagataa

SEQ ID NO: 43 - DNA, 1968 bp, Phaseolus acutifolius
  source 1..1968: mol_type = genomic DNA
atgtcttcttcatcttcttttctcttccctcaaactcaatccacagttcttccagacccttcaacctacttctcctctaaccttctttcatctccacttcccacaaactctttcttccaaaactatgttatcccaaacgggtcacaacctgagtacattcacccttacctcatcaaaactacaaactcctcactatcagcctcataccctcttctcctcttcaccacagcactcttgtaccaagcttttgtgccagatctcaccatctcttcaactcaaacacactcacaccaacaaaaccgtgtaatctcatcatttagtgaccttggtgtcactttggatattccctcctccaacctcaggttctttctctcaagaggaagcccttttataactgcttccgtctcttcttcaacatctctttctatcacaacactgcacaccatactctctttgtcttccaacaatgacaacaacaccaagtacacccttaagcttaacaacactcagacatggctcatatacacctcctcccccatccatttcaaccataatgcttcagaggttacgtccaagccattttctggcatcattcgtgtagcagtgctgccaaatcctaactacgagacaattcttgacaagcacagctcttgttaccctttgttgggtgatgcaacactagaggagccttcccgtgtggtgtatcaatggcaaaaggaagggtctggggatttgctcatgctggctcaccctcttcatgttaagcttttatcaaataataataacgggaatgttactttgctgagtgattttaagtacagaagcattgatggtgatcttgttggtgttgttggagattcatggatattgcaaacggatcgtattcctgtgacatggtattctaacaacggagtggaaaaaaattcatatgatgagattgtctcagcgcttgttaaggacgtgcaagcgcttaattcttcagcaataggaacaagttcatcttatttttatggaaagcgcgttggaagggccgcaaggttggcattgatagcggaagaagtgtcgttttcacaggttgttcccacggttacggattttcttaaaaaggccattgagccttggttagatggaactttcgaagggaacggttttctatatgaaaataaatggggtggacttgtaaccaaactggggtcaacggattcaagcgctgattttgggtttggagtttacaatgatcaccattaccatttggggtactttctatatggaattgcggttcttgcaaagattgatcccgagtggggacaaaaatacaagccacaagtttattcacttgtgacagattttatgaacttgggtcaaaggtataacagaaattatccacgtctaaggtgttttgacctttatatgttacattcttgggctgcgggagtgactgaatttgaagatggtaggaatcaagaaagtacgagtgaagctgtgaatgcatactattcagcagcgttggtgggtctggcatatggtgactcgagtcttgttgccactgggtcaacgttggtggcgttggaaattctagctgcacaaacttggtggcatgtgaaagtggaagacaacttgtacgaagaagaatttgcaaaagacaataggatagtggggattgtgtgggctaataagagggatagtaagttatggtgggccggtgcagactgtagagaatgcagacttggaatccaagtgctacccttgttgcctatcactgagacactgttctctgattctgattatgtgaaggagcttgtggaatggacatttccctctttaagtaatgaggggtggaagggaatgacctatgccttgcaaggagtttatgataagcaaacagcattgcagaatataagaacgttgaaaggttttgatgatggaaactcttacagtaatctcttgtggtggattcacagcagatga

SEQ ID NO: 44 - DNA, 2226 bp, Phaseolus acutifolius
  source 1..2226: mol_type = genomic DNA
atgtttaagaaacttggaagaaagattgaaagagaaatcacaaaacccttcaaaaataaaccacgaccaagaccatcatctccacctccaccacctcctcctcctccaccaccacttccttcatctacacctccaccgccacctccgccgccatctcctcctcctcctcttcctaagcaaccaaatgctccatttctcttccctcaagctcactccacaattctccctgacccttcaaccttctttgctccaaaccttctctcttctccactccctacaaactctttcttccaaaactatgttcttcaaaatggagacacacctgaatacattcacccctacctcatcaaatcctcaaactcctccctctccctctcctacccttctctcaacttcaactcttctttcatagcacaggttttcaaccctgacatcaccatctcttccactgagagcaaaaccaccccaggcttacacgccaggcacgtcatctcttccttcagtgatctaagtctcactttggacattccctcttcaaacctcaggttctttcttgtcaggggaagcccttttgtgacagcatcagttacatgtcccacaccactttccatcaccaccatgcatgccattctttcactctcatccaataactccctcaccaaacacaccttgcagctcaacaatggccaatcatggctcattaacacctcctcgcccatcagtttaaactacagcctttctgagattacttctggtgaattttctggcataataaggatagcagtgttgcctgattctgaccctaagtatgaggtaatcctcaataggttcagctcttgttatcctgtctctggggatgcaacattcacaaatccgttctgtgtaaagtataaatgggaaaagaaagggtggggggagttgttaatgctagctcaccctcttcaccttcagcttttgaatgatggtggtgatagtggtgtgacagttctgcataatttaaaatttagaagtattgatggagagcttgttggtgttgttggagactcctggctgctgaaaaccgacccggtttcagttacttggcattccacaagaggaataaaagaagaattccatgaagagattttttcagtgctttctgaagatgtggaagctttgaatcccttgggaataacaacaacagcatgctatttttatgggaagattatagcaagggcagcaaggttagcattgatagctgaagaggtggcttttctcgatgccatgcctgtggttaggaagttcttgaaggagatcattgagccatggttagacggaactttcagtggaaatggttttctctatgagggaaaatggggagggattgttactaaacaagggtctaaagattcaggagcagattttgggtttggtgtttataatgatcatcattacaatttggggtacttcctttatggaattgcggtgcttgcaaagattgatccagcttggggaaggaagtacaagcctcaagcctattcacttgtggcagatttcatgagcttgggaagaagatcagactctaagtacacgcgtttgaggtgttttgatctgtataaattgcactcttgggccggagggttaactgaatttgcagatggaagaaatcaggagagtactagtgaagctgtgaatgcatattattctgcagcattgatgggtctagcatatggtgacactcaacttattgcctctggatcaacacttgcagcattggaaattcatgcagctcaaatgtggtggcatttgggagagggacataaactgtacgaggaagattttacaaaagagaacaaggtggtaagtgttgtgtgggctaacaagagagatagtggactatggtttgctccttctcagtggagagaatgcaggcttggtattcatgttttaccactgtctcctattaccgaggccttgttctctgatgttggttatgtgaaggaacttgtggagtggacagtgcccaatttgaacaggaaatgtgttggagaagggtggaaggggtttatctatgccttggaaggaacttatgataaagaaagtgcagtgcaaaaggtaagaagcttgaaagtttttgatgatgggaactcaatgtctaatctgttgtggtggattcatagcaggggtgatgtggaggaggaatttggtcaaggaaaacaatgctggtttggccattactgccactaa

SEQ ID NO: 45 - DNA, 2133 bp, Phaseolus acutifolius
  source 1..2133: mol_type = genomic DNA
atgttgaaaaaacttagacgcaaggttagcacagccctgagaagtggccttaagaatgggtccaaaccctataaaaacccatcaccaccaccttcatcaccattaccacttccattagtaccagtaagaacaatgtctcatactagaaaacattctccttttttgtttccacatgttgattcctctgttgttcctgatccctccaatttcttctccccaaaccttctctcaaatcccctccccactaactctttcttccaaaactttaccttgaaaaatggtgatcaacctgagtattttcacccttacctcgtcaaatcctcaaacttttctctctccctttcatacccatctcgctcttttaactcctccttcacttaccaggtcttcaaccctgatctcaccatttcttcctctcaaaagccccacctttcccatttcaaccataccatctcttcccacaatgatctcagtgtcactttggacatcccttcttccaatttgaggtttttccttgttaggggtagcccctttttgaccctctctgtgactcaaccgacccctctttccatcaccactattcacgccattctatccttttcctccagtgattccctcacaaagcacacttttaacctcaacaatggccagacttggattttgtatgcttcttcgccgattaggttgagtcatggactttctgagataaattgtgatgcgttttctggcataattaggattgccctgttgcctgattctgattcgaagcacgaggctgtgcttgacaggttcagttcctgttaccctgtgagcggtgaggctgtgtttgccaggccattttgtgtggagtataagtgggagaagaaagggtggggtgatttgttgatgttggcacaccctctccatcttcagcttttggctgatggtggttgtgatgttaatgttctgagtgattttaagtatgggagcattgatggggaccttgttggggttgttggtgattcatggagtttgaaaactgatcctgtttctgtgacttggcactctataaggggtgtgagagaagaatcccgggatgaggttgtttcggcgcttgtgaatgatgttgagcggctgaattcatcttcaataacgacgaactcgtcgtatttttatgggaaactgattgcaagggctgcaaggttggctttgatagctgaagagatgtgttttcttgatgtgattcctaaggttaggaagtatttgaaggaaaccattgaaccgtggctggaggggacttttaatgggaatggatttctgtatgataggaaatggggtggcattgttaccaaacaagggtccaatgatgctggtgctgattttgggtttggaatttacaatgatcaccattatcatttgggatacttcgtttatggaattgcagtgcttgctaagattgatccagtgtggggtaggaagtataagcctcaagcctattctctcatggcagattttatgacactgagcagaagatcaaattcgaactacacaagactaaggtgttttgacctttataaattacactcatgggctggagggttaactgagtttgcagatggaagaaatcaggagagtaccagtgaagctgtcaatgcatactattctgctgccttgatgggtctggcatatggtgacacacaccttgttgccactggatcaacgctcacagcattggagattcatgcagctcaaatgtggtggcatgtgaaacagggagataatcactatggtgaagagtttgagagggagaacaaggtagttggtgttctttgggctaacaagagggatagtggactatggtttgcgcctcctgagtggaaagaatgtcggcttgggattcaactcttaccgttactgccgatttctgaagtgttgttctccgatgttgactttgtgaaggatcttgtggagtggacattgcctgccttgaatagggaaggtgttggagaaggatggaaagggtttgtttatgcactgcagggaatatatgataatgaagcagaagtgatgaagaggaatttggtcatggaaaacactgctggtttggtcattactgccactaggcagctgttacctgatgaatgccctctatag

SEQ ID NO: 46 - DNA, 1989 bp, Phaseolus acutifolius
  source 1..1989: mol_type = genomic DNA
atggttaagcaaaacaaaactcatttcatcttcccagagacacaatccactgtgcttcctgatccctccaacttcttctcctcaacccttctctcaaaaccactccccaccaactctttcttccaaaactttgtcctaaaaaatggtgatcaacctgaatacattcatccttacctcatcaaatcctctaactcttccctctctctctcatacccttctcgccaagtcagttctgctgtcatattccaagtcttcaacgctgatctcactatctcatccaagcaaggttccagtgggaaacacgttatctcctcctatagtgatctcagtgtcactttggatatcccttcttccaatcttagcttcctccttgttaggggaagcccctttttgactgtttctgtcacccaaccaacccctctttccatcaccaccatccacgccattctctcattctcttcaaacaagactaacaccaagtacacctttcacttcaacaatggccaaacatggatcctttattcttcctccaccatcaagttgagccacactctttctgagatcacttctgatgcattttctggcatagtccggatagccctgttgcctgattctgattcaaaacacgaggcggttcttgacaagtttagttcttgttaccccgtgtcaggtgaagctatatttagagaacccttttgtgtggagtacaagtgggagaagaaaggatcaggagatttgctactcttggcccaccctctccatgttcagcttctgtccaatggagacaatgatgttactgttctggaagattttaaatatggaagcattgatggggatgttgttggtgttgttggggattcgtgggttttgcaaacagatcccgtgtatgtaacatggcactcaaccaagggagtcaaagaagaatcccatgatgaaattgtttcagccctttctaatgatgttgaaggcctaaactcatcatcgatttcaacaacttcgtcatatttttatgggaagttgattgcaagggctgcaaggttggcattgattgctgaggagttgagctaccctgatgtgattccaaaggttaagaagtttttgaaggaaagcattgagccatggttggagggaactttcaatgggaatggatttctacatgataagaaatggggtggcattattacccaacaagggtccaatgatggtggtggtgattttggatttggtatttacaatgatcaccactaccatttggggtacttcctttatgcaattgcagtgctcgttaagcttgatccagcctggggtaggaagtacaaggctcaagcctattccattgtgcaagacttcatgaacttggacacaaaactaaacaacaattacacacgtttgaggtgttttgacctttatgtgcttcactcttgggctggagggttaactgagttcagtgatggaaggaaccaagagagcacaagtgaggctgtgtgtgcatattactctgctgctttggtggggctggcatatggtgatgctcatcttgtttcccttggatcaacactaacagcattggaaattcttgggactaaaatgtggtggcatgtggaagaggaagggagtttgtatgaggaagagttcacaagagagaacaggatcatgggggttctgtggtctaacaagagggacactggactatggtttgctcctgcagagtggaaagagtgtaggcttggcattcagctcttaccattggtacctatttctgaagccattttctccaatgctgagtatgtgaagcagcttgtggagtggactttgcctgctttgaatagggatggtgttggtgaaggatggaagggatttgtatatgcccttgaagggatttatgacaatgaaagtgcattgcagaagataagaaaccttgcaggttttgatggtggaaactctctcagtaatctcttgtggtggattcacagcataggaaatgaatga

SEQ ID NO: 47 - DNA, 1977 bp, Cicer arietinum
  source 1..1977: mol_type = genomic DNA
atgtcatcttcttctgttcctttcctctttccccaaactcattccacaatcctcccaaacccatcaaacttcttctcaccaaatctgctatccacacccctccctacaaactctttcttccaaaactttgttcttcaaaatggtgaccaacctgaatacattcacccttacctcatcaaatcctcaaactcttctctctcattttcataccctcttctcttattcacaacatcatttttataccaagtttttgttccagatctcactatttcttcctcacaaaaaacaacatcaaaaaacaaacatgttatttcatcatatagtcatcttagtgtgactcttgaaatcccttcttcaaatttaagattttttcttgttagaggaagcccttttataactgcaaatgttacaaaaccaacttcactttcaatcacaacactaaataaaatagtttctttctcttcttttgattacaaaaaaaccaaacacacccttcaactcaataacactcaaaaatggattatatacacttcttcaccaatcaatttcaaccatgatggttttgaggttatatcgaatccattttcgggtattattcgtatcgcgattgttcctaattcaaatcctttttatgagaaaactcttgataagttcagttcttcttatcctgtttctggtgatgcaaacattaagaaaaattttagtttggtttataattttcaaaagaaaaggttgggtgatttacttatgctagctcatcctcttcatgttaagcttctatcaaatgatgttaaagttttgcatgattttaagtataaaagtgttgatggtgatcttgttggtgttgttggagattcatggttattgaaaaatgatcctgtttctgtgaattggtattctaataaaggtgttgcaaaagaatcacataatgagattgtttcagctcttattaaagatgtgaatgagttgaatttgtcgtcgatttcaacaacttcatcttatttttatggaaagattgttggtagagctgcaagatttgctttgatagctgaagaagtttcttatcttaaagtgattccaaagattaaattttttttgaaggaaactattgagccatggttgaatggaaatttcaaaggaaatggttttttatatgagaaaaaatggggtggacttgttactcaacaagggttaaatgattcaagtgctgattttggttttggagtttacaatgatcatcattaccatttgggttattttctttatggaatttcagttcttgtaaaaattgatcctttatggggacaaaagtataaacctcaagtttattcacttttgaaagattttatgaatttgggtgagagagataataaaaattatccaagtttaaggtgttttgatcattacaagttacattcttgggcttcagggttgactgaatttgaaaatggaaggaatcaagaaagttcaagtgaagctgtgaatgcatattattcagctgcattaataggtttagcatatggtgattcaaaaattgttgaaattgggtcaacaattttagcatttgaaattaaggctgcacaaacttggtggcatgtgaaattggaaaataatttgtatggtgaagattttgcaaaagagaatagaatagttggtattttatgggctaataaaagagatagtaaactttggtgggccccatctgaatgtagagagtgtagacttagtatacaagttttacctttgttgccaattagtgaaactttgttttttgatggtgtttatgctaaggagttagttgaatggacattaccttctttgaagaataaaactaatgttgaagggtggaaagggttcacttatgctttggaagggatttatgataaggaaatagcattgaagaatattagaggattgaaaggttttgatgatggaaactcatttactaatcttttgtggtggattcatagtagatga

SEQ ID NO: 48 - DNA, 2022 bp, Cicer arietinum
  source 1..2022: mol_type = genomic DNA
atgtctactatcaaaaagaacactccttttatcttcccacagacaaattcaactgtcctccctgacccctccaacttcttctctccaaatttgctatccacacccctccctacaaactctttcttccaaaacttttctctcaaaaatggtgaccaacctgaatacattcatccttacctcatcaaatcatcaaactcatcactttctgtctcatacccttctcatttttccaattcatctttcatataccaagttttcaatgctgatctcaccataacttccttagaacaaaaaaccaatcaaacttccaatgaaaaacacataatatcttcttatagtgatcttagtgtcaccttagatatcccttcatcaaatctaagtttctttcttgttagaggaagtccttatttaactttttctgtaacaaaaccaacacctctttccatttccaccattcatgccattgaattcttagtccctacagatccatccattaccaggtacacctttcagcttaacaatggtcaaacatggcttttatatgcttcctcgccgatcaagttgagccatgatctttctgagatcacctgtgagcctttttctggtgtaattcggatcgctttgttgccgaataacgatcgtaaaattgaagatgttcttgagaagtatagttcttgttaccctttatcaggtgatgcttttcttagagaaccattttgcgttgagtataaatggcagaagaatggttcaagtgatttgctactattagcacaccctcttcatgttaagcttttgtctaatagtgaaagtgatgttacttttttgaatgatttgaagtatacaagcattgatggtgatcttgttggtgttgttggtgattcatggattttgaaaacagaacctgtttcaataacttggcactcaagcaaaggtgtaaaagaagaatcgcatgacgaaattgtttcatcgctttcgaaagatgttgaaggtttaaactcatcagcaataacaacaacatcatcatatttttatggaaaattgattgcaagagcagctaggcttgcattgattgctgaagaagtttttttctttgatgccattcaaaaagttaggaactttttgaaggaaacaattgaaccatggcttgaaggaactttcaatggaaatggatttctatatgatagaaaatggggtggcattataactcaacaagggtctaatgatagtaatggcgatttcggtttcggaatttacaatgatcatcattatcatttaggatattttctttatgcaattgctgttcttgttaagattgatccaacatggggtaggaagtataaaactcaagcttattcacttatggaagattttatgaacttgaacataagattaaactcgaattatacgcggttaaggtgtttcgatctttacaagttacattcttgggctggagggttaactgagttttctgatggaaggaatcaagagagtactagtgaagctgtgaatgcatactatgctgcagcattgatgggaatggcatatggtgatgcttcacttgtgagcattggatcaaccttaacatcattggaaattcttggaacaaaaatgtggtggcatgtgaaaaaggaagggaaattgtatgaagaagagtttacaaaagagaataggataatgggagttttgtggtctaataagagagatagtggactttggtttgcagcggctgagtctagagaagctaggcttggaattcagcttataccattgagtccaatttctgaagttttgttttctgatgttagttatgtgaaggatcttgtggagtggactttgcctgctttgaatagggaaggtgttggtgaaggatggaagggttttttgtactcattgcaaggtgtttatgataatcaaggtgcattggagaagataagaaatttgaatggttttgatggtggtaactctttgactaatcttttgtggtggattcatagcagaggtgaagatggtgatgatgagtaa

SEQ ID NO: 49 - protein, 663 aa, Medicago truncatula
MSSSSSLPFLFPQTHSTVLPNPSNFFSQNLLSTPLPTNSFFQNFVLHNGDTPEYIHPYLIKSSNFSLSISYPLLLFSATMLYQVFSPDLTISSSQKSHTNTTKNHVISSYSDLGVTLDIPSSNLRFFLVRGSPFITASVTKPTSLSITTLHNIVSLSSFDDKNTKHTLQLNNTQKWIIYTSSPIKFNHDGSEIVSNPFSGIIRIIVIPNTKFEKILDKFSSCYPVSGDANIKNKFHLEYKWQKKCSGDLLMLAHPLHVKLLSQSNNVNVTVLHDLKYTSVDGDLVGVIGDSWILETDPVNVTWYSSKGVTKESHDEIVSALVKDVKELNSSAITTNGSYFYGKIVSRAARFALIAEEVSYPKVIPIIKNFLKETIEPWLNGTFKGNGFLYEKKWGGLVTKQGVNNSVVDFGFGIYNDHHYHLGYFLYGIAVLAKIDPFWGQKYKPQAYSLLQDFMNLGQRDNKNYPTLRCFDFFKLHSWAAGVTEYENGRNQESSSEAVNAYYSAALIGLAYGDKDLVAIGSTLLALEINATQTWWHVKVENNLYGEEFAKENRIVGILWANKRDSKLWWAPSECRGCRVSIQVMPLLPITESLFNDGVYAKELVEWTLPSLKNDTNDDRWKGFIYSLQGIYDKENALKKIRMLEGFANGNSFSNLLWWIHSR

SEQ ID NO: 50 - protein, 663 aa, Medicago sativa
MSSSSSLPFLFPQTHSTVLPNPSNFFSQNLLSTPLPTNSFFQNFVLYNGETPEYIHPYLIKSSNFSLSVSYPLLLFSTAMLYQVFSPDLTISSSQKTHTNIPKNHVISSYSDLGVTLDIPSSNLRFFLVRGSPFITASVTKPTPLSITTIHSIISLSPFDKKKTKYTLQLNNNQKWIIYTSSPIKFNHDGSEVMSNPFSGIIRIVIVPNSKYEQVLDKFSTCYPVSGDANIKNKFHLEYKWQKKCSGDLLMLAHPLHVKLLSQSNDASVTVLHDLKYTSIDGDLVGVIGDSWILETNPVNVTWYSSKGVTKESHDEIVSALVKDVKELNSSAITTNGSYFYGKIVSRAARFALIAEEVSYPKVIPIIKNFLKETIEPWLNGTFKGNGFLYEKKWGGLVTQQGVNDSGVDFGFGIYNDHHYHLGYFLYGIAVLAKIDPFWGQKYKPQTYALVKDFMNLGQRDNKNYPTLRCFDFFKLHSWAAGVTEYENGRNQESSSEAVNAYYSAALIGLAYGDKDLVDIGSTLLAFEINATQTWWHVKVEKNLYGEEFAKENRIVGILWANKRDSKLWWAPSECRGCRVSIQVMPLLPITESLFNDGVYAKELVEWTLPSLKNETNDDRWKGFIYALQGIYDKENALKKIRMLESFANGNSFSNLLWWIHSR

SEQ ID NO: 51 - protein, 663 aa, Medicago sativa
MSSSSSLPFLFPQTHSTVLPNPSNFFSQNLLSTPLPTNSFFQNFVLHNGETPEYIHPYLIKSSNFSLSVSYPLLLFSTAMLYQVFSPDLTISSSQKTHTNIPKNHVISSYSDLGVTLDIPSSNLRFFLVRGSPFITASVTKPTPLSITTIHSIISLSPFDKKKTKYTLQLNNNQKWIIYTSSPIKFNHDGSEVMSNPFSGIIRIVIVPNSKYEQVLDKFSTCYPVSGDANIKNKFHLEYKWQKKCSGDLLMLAHPLHVKLLSQSNDASVTVLHDLKYTSIDGDLVGVIGDSWILETNPVNVTWYSSKGVTKESHDEIVSALVKDVKELNSSAITTNGSYFYGKIVSRAARFALIAEEVSYPKVIPIIKNFLKETIEPWLNGTFKGNGFLYEKKWGGLVTQQGVNDSGVDFGFGIYNDHHYHLGYFLYGIAVLAKIDPFWGQKYKPQTYALVKDFMNLGQRDNKNYPTLRCFDFFKLHSWAAGVTEYENGRNQESSSEAVNAYYSAALIGLAYGDKDLVDIGSTLLAFEINATQTWWHVKVEKSLYGEDFAKENRIVGILWANKRDSRLWWAPSECRGCRLSIQVMPLLPITESLFNDGVYAKELVEWTLPSLKNETNDDRWKGFIYALQGIYDKENALKKIRMLEGFANGNSLSNLLWWIHSR

SEQ ID NO: 52 - protein, 663 aa, Medicago sativa
MSSSSSLPFLFPQTHSTVLPNPSNFFSQNLLSTPLPTNSFFQNFVLHNGETPEYIHPYLIKSSNFSLSVSYPLLLFSTAMLYQVFSPDLTISSSQKTHTNIPKNHVISSYSDLGVTLDIPSSNLRFFLVRGSPFITASVTKPTPLSITTIHSIISLSPFDKKKTKYTLQLNNNQKWIIYTSSPIKFNHDGSEVMSNPFSGIIRIVIVPNSKYEQVLDKFSTCYPVSGDANIKNKFHLEYKWQKKCSGDLLMLAHPLHVKLLSQSNDASVTVLHDLKYTSIDGDLVGVIGDSWILETNPVNVTWYSSKGVTKESHDEIVSALVKDVKELNSSAITTNGSYFYGKIVSRAARFALIAEEVSYPKVIPIIKNFLKETIEPWLNGTFKGNGFLYEKKWGGLVTQQGVNDSGVDFGFGIYNDHHYHLGYFLYGIAVLAKIDPFWGQKYKPQTYALVKDFMNLGQRDNKNYPTLRCFDFFKLHSWAAGVTEYENGRNQESSSEAVNAYYSAALIGLAYGDKDLVAIGSTLLAFEINATQTWWHVKVEKYLYGEEFAKENRIVGILWANKRDNNLWWAPSECRGCRLSIQVMPLLPITESLFNDGVYAKELVEWTFPSLKNETNDDRWKGFIYALQGIYDKENALKKIRMLEGFANGNSFSNLLWWIHSR

SEQ ID NO: 53 - protein, 663 aa, Medicago sativa
MSSSSSLPFLFPQTHSTVLPNPSNFFSQNLLSTPLPTNSFFQNFVLHNGETPEYIHPYLIKSSNFSLSVSYPLLLFSATMLYQVFSPDLTISSSQKTHTNIPKNHVISSHSDLGVTLDIPSSNLRFFLVRGSPFITASVRKPTSLSITTLHNIVSLSSFDDKNTKYTLHLNNTQQWIIYTSSPIKFNHDGSEIVSNPFSGIIHIVVVPSSKYEKILDKLSSCYPVSGDANIKNRFHLEYKWKKKCSGDLLMLAHPLHVKLLSQSNNVNVTVLHDLKYTSVDGDLVGVIGDSWILKTDPVNVTWYSSKGVTKESHDEIVSALVNDVKELNSSAITTNGSYFYGKIVSRAARFALIAEEVSYPKVIPIIKNFLKETIELWLNGTFKGNGFLYEKKWGGLVTKQGVNNSGVDFGFGIYNDHHYHLGYFLYGIAVLAKIDPFWGQKYKPQIYALVKDFMNLGQRDNKNYPTLRCFDFFKLHSWAAGVTEYENGRNQESSSEAVNAYYSAALIGLAYGDKDLVAIGSTLLAFEINATQTWWHVKVENNLYGEEFAKENRIVGILWANKRDSKLWWAPSECRGCRVSIQVMPLLPITETLFNDGVYAKELVEWTLPSLKNETNDDRWKGFIYALQGIYDKGNALKNIRMLEGFANGNSFSNLLWWIHSR

SEQ ID NO: 54 - protein, 660 aa, Medicago sativa
MSSVPFLFPQTHSTVLPNPSNFFSQNLLSTPLPTNSFFQNFVLQNGDQHEYIHPYLVKSSNFSVSVSYPLLLFSTAMLYQVFSPDLTISSSQKTHTNIPKNHVISSYSDLGVTLDIPSSNLRFFLVRGSPFITASVTKPTPLSITTIHSIISLSPFDKKKTKYTLQLNNNQTWIIYTSSPINLNHDGSEVKSGPFSGIIRIAVVPDSNGEKILDKFSSCYPVSGDANIKKKFGLVYKWQRKNSGDLLMLAHPLHVKLLSKSNNHGVTVLDDFKYKSVDGDLVGVVGNSWNLKTDSVNVTWHSNKGVTKESHAEIVSALVNDVKKLNFSSITTNSSYFYGKIVGRAARFAFIAEEVSYPKVIPIIKNFLKETIEPWLDGNFKGNGFFYEKSWGGFVTQQGINDSSADFGFGIYNDHHYHLGYFLYGIGVLAKIDPSWGQKYKPQVYSLVKDFMNLGQRDNKNYPTLRCFDPYKLHSWASGLTEFEHGRNQESSSEAVNAYYSVALVGLAYGDKDLVATGSTLLALEINAVQTWWHVKFENNLYGGDFAKGNRIVGILWSNKRDSALWWAASECRECRLSIQVLPLLPITESLFNDGVYAKELVEWTVPSFKNKTNIEGWKGFTYALQGVYDKKNALKNIRMLKGFDDGNSFSNMLWWIHSR

SEQ ID NO: 55 - protein, 660 aa, Medicago sativa
MSSVPFLFPQTHSTVLPNPSNFFSQNLLSTPLPTNSFFQNFVLQNGDQHEYIHPYLVKSSNSSLSVSYPLLLFSTAMLYQVFSPDLTISSSQKTHTNIPKNHVISSYSDLGVTLDIPSSNLRFFLVRGSPFITASVTKPTPLSITTIHSIISLSPFDKKKTKYTLQLNNNQTWIIYTSSPINLNHDGSEVKSGPFSGIIRIAVVPDSNGEKILDKFSSCYPISGDANIKKKFGLVYKWQRKNSGDLLMLAHPLHVKLLSKSNNHGVTVLDDFKYKSVDGDLVGVVGNSWNLKTDSVNVTWHSNKGVTKESHAEIVSALLNDVKKLNISSITTNSSYFYGKIVGRAARFALIAEEVSYPKVIPIIKNFLKETIEPWLDGNFKGNGFFYEKSWGGLVTQQGINDSSADFGFGIYNDHHYHLGYFLYGIGVLAKIDPSWGQKYKPQVYSLVKDFMNLGQRDNKNYPTLRCFDPYKLHSWASGLTEFEHGRNQESSSEAVNAYYSVALVGLAYGDKDLVATGSTLLALEINAVQTWWHVKVENNLYGQDFAKENRIVGILWANKRDSALWWAASECRECRLSIQVLPLLPITESLFNDGVYAKELVEWTVPSFKNKTNIEGWKGFTYALQGVYDKKNALKNIRMLKGFDDGNSFSNMLWWIHSR

SEQ ID NO: 56 - protein, 584 aa, Medicago sativa
MLYQVFSPDVTISSSQKTHTNIPKNHVISSYSDLGVTLDIPSSNLRFFLVRGSPFITASVTKPTPLSITTIHSIISLSPFDKKKTKYTLQLNNNQTWIIYTSSPINFNHDGSEVKSGPFSGIIRIAVVPDSNGEKILDKFSSCYPVSGDANIKKKFGLVYKWQRKNSGDLLMLAHPLHVKLLSKSNNHGVTVLDDFKYKSVDGDLVGVVGNSWNLKTDSVNVTWHSNKGVTKESHAEIVSALVNDVKKLNFSSITTNSSYFYGKIVGRAARFALIAEEVSYPKVIPIIKNFLKETIEPWLDGNFKGNGFFYEKSWGGLVTQQGINDSSADFGFGIYNDHHYHLGYFLYGIGVLAKIDPSWGQKYKPQVYSLVKDFMNLGQRDNKNYPTLRCFDPYKLHSWASGLTEFEHGRNQESSSEAVNAYYSVALVGLAYGDKDLVATGSTLLALEINAVQTWWHVKVENNLYGQDFAKENRIVGILWANKRDSALWWASSECRECRLSIQVLPLLPITESLFNDGVYAKELVEWTVPSFKNKTNIEGWKGFTYALQGVYDKKNALKNIRMLKGFDDGNSFSNMLWWIHSR

SEQ ID NO: 57 - protein, 584 aa, Medicago sativa
MLYQVFSPDLTISSSQKTHTNIPKNHVISSYSDLGVTLDIPSSNLRFFLVRGSPFITASVTKPTPLLITTIHSIISLSPFDKKKTKYTLQLNNNQTWIIYTSSPINFNHDGSEVKSGPFSGIIRIAVVPDSNGEKILDKFSSCYPISGDANIKKKFGLVYKWQRKNSGDLLMLAHPLHVKLLSKSNNHGVIVLDDFKYKSVDGDLVGVVGNSWNLKTDSVNVTWHSNKGVTKESHAEIVSALVNDVKKLNFSSITTNSSYFYGKIVGRAARFALIAEEVSYPKVIPIIKNFLKETIEPWLDGNFKGNGFFYEKSWGGLVTQQGINDSSADFGFGIYNDHHYHLGYFLYGIGVLAKIDPSWGQKYKPQVYSLVKDFMNLGQRDNINYPTLRSFDPYKLHSWASGLTEFEHGRNQESSSEAVNAYYSVALVGLAYGDKDLVATGSTLLALEINAVQTWWHVKVENNLYGQDFAKENRIVGILWANKRDSALWWASSECRECRLSIQVLPLLPVTESLFNDGVYAKELVEWTVPSFKNKTNIEGWKGFTYALQGVYDKKNALKNIRMLKGFDDGNSFSNLLWWIHSR

SEQ ID NO: 58 - protein, 658 aa, Pisum sativum
MSSPPYLFPQTQSTILPNPSNFFSPNLLSTPLPTSSFFQNFALKNGDQPEYIHPYLIQSSNSSLSVSYPLLLFSTALLYQVFSPDLTISSTQKPQTNIPQNNHVISSYSDLGVTLDIPTANLRFFLVRGSPFVTALVTKPTPLSIKTNHTIVSFSSFDYKKTKYRLSLNNGQKWIIYTSSPINFNHDGSEVKSDPFSGIIRFAVVSNSNNEKILHEFSSSYPVSGYAKIEDKFGLVYKWKTKNSGDLLMLAHPLHVKLLSKNSNDHKVTILNDFKYRSVDGDLVGVVGKSWLLKTDSVNVTWHSSKGVSKDSYEEVVSALEKDVNELNVATINTTSSYFYGKIVARAARLALIAEEVSYEKVIPIVKDFLKKTIEPWLDGNFKGNGFLYEKTWGGLVTQQGVNDSGADFGFGVYNDHHYHLGYFLYGIGVLAKLDQDWGQKYKPIVYSLLKDFMNLGQRDNKNYPTLRSFDPYKLHSWASGLTEFRDGRNQESTSEAVNAYYSVTLVGLAYDDEDLVAIGSTLLAFEINAAQTWWHVKAENNVYGTDFAKQNPVVGVLWANKRDSSLWWASSECRQCRLSIQVLPLLPITENLFNDGVYAKELVEWTWPTLSKEGWKGFTYALQGVYDKENALKNIRTLKGFDDGNSLSNLLWWIHSR

SEQ ID NO: 59 - protein, 660 aa, Pisum sativum
MCSPPYLFPQTQSTILPNPSNFFSQDLLSTPLPTNSFFQNFALKNGDQPEYIHPYLIQSSNSSLSVSFPLLFFSTALLYQVFTPDLTISSTQKPQTNIPQNNHVISSYSDLGVTLDIPTTNLRFFLVRGSPFVTAQVTKPTPLSIKTIHAILSFSSFDNKKNKYALSLNNGQKWIIYTSSPINFNHDISEVKSDPFTGVIRIAAVSDSNNEKILDEFSSSYPVSGHAIVDVKNKFGLVYKWETENSGDLLMLAHPLHVKLLSKNSNDHKVTILNDFKYRSVDGDLVGVVGNSWLLKTDTINVTWHSSKGVAKESYEEVVSALEKDVNELNVASISTTSSYFYGKIVARAARFALIAEEVSYEKVIPIVKDFLKKTIEPWLDGNFKGNGFLYEKTWGGLVTQQGVNDAGADFGFGVYNDHHYHLGYFLYGVGVLAKLDQDWGQKYKPIVYSLLKDFMNLGQGDNKNYPTLRSFDPYKLHSWASGLTEFSDGRNQESTSEAVNAYYSAALVGLAYGDEDLVAIGSTLLALEINAAQTWWHVKTENNVYGADFAKQNSVVGVLWANKRDSSLWWASSECRECRLSIQVLPLLPITENLFNDGVYVKELVEWTWPTLSNEGWKGFTYALQGVYDKENALNNIRALKGFDDGNSLSNILWWIHSR

SEQ ID NO: 60 - protein, 654 aa, Vicia faba
MSSPPYLFPQTQSTILPNPSNFFSQNLLSTPLPTNSFFQNFALKNGDQPEYIHPYLIQSSNSSLSVSYPLLLFSTALLYQVFSPDLTISSTQQPQTNINHVISSYSDLGVTLDIPTSNLRFFLVRGSPFVTALVTKPTPLSIKTIHTIVSFSTFDNKKTKYTLSLNNTQKWIIYTSSPINFNHLGSEVISDPFSGIIRIASVSNSNNEKILDEFSSSYPVSGYAKIENKFGLVYKWETQNSGDLLMLAHPLHAKLLSNSKDHKVTILNDFKYRSIDGDLVGVVGNSWLLKTDSFNVTWHSSKGVTKESYEEVVSALEKDVNELNVASITTTSSYFYGKIVARAARFALIAEEVSYEKVIPVVKGFLKQTIEPWLDGKFKGNGFLYEKTWGGLVTQQGVNDVGADFGFGVYNDHHYHLGYFLYGIGVLAKIDQDWGQKYKPIVYSLLKDFMNLGLGDNPNYPTLRNFDPYKLHSWASGLTEFRDGRNQESTSEAVNAYYSVTLIGLAYGDEDLVVVGSTLLALEINAAQSWWHVKAENNVYGTDFAKQNPIVGVLWANKRDSSLWWASSACRECRLSIQVLPLLPITENLFNDGVYAKELVEWTLPTLSNEGWKGFTYALQGVYDKENALKNIRTLKGFDDGNSLSNLLWWIHSR

SEQ ID NO: 61 - protein, 661 aa, Trifolium pratense
MSSVPFLFPQTHSTVLPNPSNFFSQNLLSTPLPTNSFFQNFVLKNGDQPEYIHPYLIKSSNFSLSVSYPFLLFSTAMLYQVFSPDLTISSSQKSHTNSQKNKHFISSYSDLGVTLDIPSSNLRFFLVRGSPFVTASVTKPTPLSITTLHNIVSLSCFDNKKTKYTLLLNNTQKWIIYTSSPINLNHDGSEVKSGPFSGIIRIAVVPDSNYEKILDKFSSCYPVSGYANIQKKFGLVYKWQRKNSGDLLMLAHPLHVKLLSKSNNHGVTVLNDFKYRSVDGDLVGVVGNSWNLKTDPIDVTWHSSKGVTKESHDEIVSALVKDVKKLNISAIETNSSYFYGKIVGRAARFALIAEEISYFKVIPIIKNFLKKTIEPWLDGNFKGNGFFYEKSWGGLVTQQGINDSSADFGFGVYNDHHYHLGYFLYGIGVLAKIDPLWGQKYKPIVYSLLKDFMNLGKRDNKNYPTLRCFDPYKLHSWASGVTEFENGRNQESSSEAVNAYYSAALVGLAYNDKNLVATGSTLLALEINAVQTWWHVKAESNLYGEDFAKENRIVGILWANKRDSKLWWAPSECRECRLSIQVLPLLPITETLFNDGVYAKELVEWTLPSLKNKTNVEGWKGFTYALQGVYDNKNALKKIRLLKGFDDGNSFSNLLWWIHSR

SEQ ID NO: 62 - protein, 661 aa, Trifolium repens
MSSVPFLFPQTHSTVLPNPSNFFSQNLLSTPLPTNSFFQNFVLNNGDQPEYIHPYLIKSSNSSLSVSYPFLLFSTAMLYQVFSPDLTISSSQKSHSNSPKNKHVISSYSDHGVTLDIPSSNLRFFLVRGSPFVTAYVTKPTPLSITTLHNIVSLSSFDNKKTKFTLLLNNTQKWIIYTSSPINLNHDGSEVKSDPFSGIIRIAVVPDSNYEKILDKFSSCYPVSGYANIQKKFGLVYKWQTKNSGDLLMLAHPLHVKLLSKSNNHGVIVLNDFKYRSVDGDLVGVVGNSWNLKTDSIDVTWHSSKGVTKESHDEIVSALVKDVKELNISSIATNSSYFYGKIVGRAARFALIAEEVSYFKVIPIIKNFLKETIEPWLDGNFKGNGFFYEKSWGGLVTQQGINDSSADFGFGVYNDHHYHLGYFLYGIGVLAKIDPLWGQKYKPRVYSILKDFMNLGQRDNKNYPTLRCFDPYKLHSWASGATEFENGRNQESSSEAVNAYYSAALVGLAYNDKNLVATGSTLLALEINAAQTWWHVKVENNLYGEDFAKENRIVGILWANKRDSKLWWAPSECRECRLSIQVLPLLPITETLFNDGVYAKELVEWTLPSLKNKTNVEGWKGFTYALQGVYDKKNALKKIRMLKGFDDGNSFSNLLWWIHSR

SEQ ID NO: 63 - protein, 479 aa, Trifolium repens
MSSVPFLFPQTHSTVLPNPSNFFSQNLLSTPLPTNSFFQNFVLNNGDQPEYIHPYLIKSSNSSLSVSYPFLLFSTAMLYQVFSPDLTISSSQKSHSNSSKNKHVISSYSDLGVTLDIPSSNLRFFLIRGSPFVTALVTKPTPLSITTLHTIVSLSSFDNKKTKFTLLLNNTQKWIIYTSSPINLNHDGSEVKSDPFSGIIRIAVVPDSNYEKILDKFSSCYPVSGYANIQKKFGLVYKWQTKNSGDLLMLAHPLHVKLLSKSNNHGVIVLNDFKYRSVDGDLVGVVGNSWNLKTDSIDVTWHSSKGVTKESHDEIVSALVKDVKELNISSIATNSSYFYGKIVGRAARFALIAEEVSYFKVIPIIKNFLKETIEPWLDGNFKGNGFFYEKSWGGLVTQQGINDSSADFGFGVYNDHHYHLGYFLYGIGVLAKIDPLWGQKYKPRVYSILKDFMNLGQRDNKNYPTLRCFDPYKLHSWAS

SEQ ID NO: 64 - protein, 661 aa, Trifolium subterraneum
MSSVPFLFPQTHSTVLPNPSNFFSQNLLSTPLPTNSFFQNFVLNNGDQPEYIHPYLIKSSNSSLSVSYPFLLFSTAMLYQVFSPDLTISSSQKSHSNSTKNKHFISSYSDLGVTLDIPSSNLRFFLVRGSPFVTASVTKPTPLSITTLHNIVSLSSFDNKKTKYTLLLNNTQKWIIYTSSPINLNHDGSEVKSDPFSGIIRFAVVPNSNYEKILDKFSSCYAVSGYANIQKKFGLVYKWQRKNSGELLMLAHPLHVKLLSKSNNHGVTVLNDFKYRSVDGDLVGIVGNSWNLKTDSIDVTWHSSKGVTKESHDEIVAALVKDVKELNISAIETNSSYFYGKIVGRAARFALIAEEVSYFKVIPIIKNFLKKTIEPWLDGNFKGNGFFYEKSWGGLVTQQGINDSSADFGFGVYNDHHYHLGYFLYGIGVLAKIDPLWGQKYKPIVYSLLKDFMNLGQRDNKFYPTLRCFDPYKLHSWASGVTEFENGRNQESSSEAVNAYYSAALVGLAYNDKNLIATGSTLLALEINAAQTWWHVKVENNLYGEDFAKENRIVGILWANKRDSKLWWAASECRECRLSIQVLPLLPITETLFNDGVYAKELVDWTLPSLKNKTNVEGWKGFTYALQGVYDKKNALKKIRMLKGFDDGNSFSNLLWWIHSR

SEQ ID NO: 65 - protein, 723 aa, Lotus japonicus
MIFITNNGSKGNTYARSFILTSKVNFHQSSLVSNLTKNSYKKTTPPHKQLHQHLIFSPPTTMPPSSPFLFPQTQSTVLPNPSTFFSQNLLSSPLPTNSFFQNLVIQNGSQPEYIHPYLIQSSNSSLSASYPLLFFSAALLYQTFVPDLTISSTIKTSNPQNHVISSYSDLGVTLDIPSSNLRFYLARGSPYITASVTKPTPLSITTVHSIVSLLSAADKTKHTLQLNNNQTWLIYSSAPINLNKHGSSELQSDPFSGVIRIAVVPDSTSNPKYEEVLDKFSSCYPVSGDAKLKGNFTVVYKWQRKNSGDLLMLAHPLHLKLLSKNKLAATVLYDFKYRSVDGDLVGVVGDSWVLEAEPVPVTWHSNRGIKKESYGEIVSALLKDVKELNYSAVATNSSYFYGKLVGRAARFALIAEEVSFPKVIPKIVKFLKESIEPWLDGTFKGNGFLYETKWGGLVTQQGSKDAGADFGFGIYNDHHFHLGYFLYGIAVLAKIDPAWGQKYKPQAYALVNDFMNLGQRYYTFSPRLRCFDPYKMHSWASGLTEFENGRNQESTSEAVNAYYSAALMGLAYGDTRLATTGSTLTALEIGATQMWWHVKKEQILYPEEFAEDNRIVGILWANKRDSNLWWAPAECRECRLSIQVLPLLPVTESLFSDAGYAKELVEWTLPSLKSKSNVEGWKGFTYSLQGIYDKEIALKSIRMLKGFDDGNSYSNLLWWIHSR

SEQ ID NO: 66 - protein, 663 aa, Lupinus angustifolius
MSSPPFLFSQTQSTVLPNPSTFFSQNLLSSPLPTNSFFQNFVLKNGDQPEYIHPYLIKSSNSSLSVSYPFLLFTTAMLYQVFVPDLTISTSSSSHKSETKTSHVISSYSDLGVTLDIPSSYLRFFLVRGSPFITTSVTKPTTLSITTTNKIVSLHSFNDKTKHTLQLQNNQTWLIYTSYPIVFYHKDYAIESNKFSGIIRFAAWPDSTPKYEEILDKFSSCYPVSGDATIKNPFRVVYKWQRKRSGELLMLAHPLHVKLLSSSLAFNNVTVLNDFKYRSVDGDLVGVVGDSWVLETEHVPITWHSKNGVKKESYNEIVSALFKDVKELNASNVTTNSSYFYGKLVGRAARLALIAEEVSYLEVIPKISDFLKEMIQPWLDGNFKGNGFLYERKWGGLVTKQGSIDAGADFGFGIYNDHHFHLGYFLYGIAVLAKIDPAWGQKYKPQAYALVTDFLNLGQRFNSYSPRLRCFDLYKLHSWASGITEFEDGRNQESTSEAVNAYYAAALLGLAYRDTRLVATASTLTALEILAAQTWWHVKSEDKLYDEEFTKDNRIVGILWANKRDSKLWWASSECRECRLSIQVLPLVPVTESLFSDAGYVKELVEWTLPSLKNKSNVDGWKGFTYALQGIYDKENSLKKIRMLKGFDDGNSFSNLLWWIHSR

SEQ ID NO: 67 - protein, 656 aa, Lupinus angustifolius
MAAPTPFLFPATQPTILPDPSTFFSSNLSSPLPTNSFFQNFVLNSGEQPEYIHPYLVKSTKNSLSIAYPLLLFTASVFYQTFAPDLTISSATPQESAAKNHVISSYSDLGVTLDIPSSNLRFFLVRGSPYITASVTKPTTLSIKTTSPIESLNPSKDNTKYILKLKSGQTWIIYSSSAISLTKGETEISSNSFSGIIRFASLRNPQQESTLDKYSSSYPVSGYAVFNKSFNVVYNLEKEGNGDLLLLAHPLHVKLLSSKSNKVTVLSDFKYPSVDGELVGVVGDSWELETKHVPLTWNSVKGVKKEAYEEIVKALVNDVNELNSSNVTTSSSYFYGKLVARAARLALIAEEVSNSEVIPKITKFLKDTIQPWLDGSFKGNSFLYEKKWGGLVTKQGSTDKGADFGFGVYNDHHYHLGYFIYGIAVLAKIDTAWGQKYKPQAYALVSDFLNTDLKSNSHYPLLRNFDVYKLHSWASGLTEFADGRNQESTSEAVNAYYAAALMGVAYHDMDLVRIASTVTALEIHAAQTWWHVKSGDKLYAEEFAKGNKIVGIVWSNKRDSSLWWASAEAKECRLSIQVLPLSPITEALFSDAAYVKELVEWTLPSLNKPNIEGWKGFTYALQGIYDKSSSLEKIRALKGVDDGNSFTNLLWWIHSR

SEQ ID NO: 68 - protein, 689 aa, Lupinus albus
MQQSLYKSKKSPLPFHMHILSSISMAHNLQHEPFLFPLTHSTVLPDPSNFFSPNLLSTPLPTNSFFQNFALKNGDQPEYIHPYLIKSSNSSLSVSYPSHFFTTAFIYQVFIADLTISASVKTNSDSIHKHVISSYNDLSVTLDFPSSNLRFFLVRGSPFLTANVTSSTPLSITTIHAILSFSSSDSLTKFTLKLNNSQTWLIYSSSPMKFSHTLSGISSDAFCGVIRIAVLPESKNSKFEEILDRFSSCYPISGDAILKKPFSVVYKWEKKGLGDLLLLAHPLHLQMLSKKNSDVTILDEFKYKSIDGDLVGVVGDSWLLKTKPVYVTWHSIQGVKKESYSEIVSALSKDVEGLNSAAITTASSYYYGKLVARAARLALIAEEIGFRDAISAITKFLKESIEPWLDGTLEENGFLYDEKWGGLVTKQGSIDSGADFGFGIYNDHHYHLGYFLYGIAVLVKIDPSWGIKYKPQAYSLMQDFMNLGEKSNSNYPTLRCFDLYKLHSWAGGLTEFADGRNQESTSEAINAYYSAALLGLAYNDTNIFETASTFASLEIHAAKTWWHVKFGDNLYEEDFTKENRIMGVLWSNKRDSGLWFAPPAMKECRVGIQLLPLVPISEMLFSNVSFVKELVKWTLLALDRNDVEDEWKGFVYALQGIYDNESALQKIRRLKGFDDGNSFTNLLWWIHSR

SEQ ID NO: 69 - protein, 656 aa, Lupinus albus
MSVPTPFLFPSIQSTVLPDPSSFFSPNISSPLPTNSFFQNLVLNGGGQPEYFHPYLINSTKTSLSVAYPLLLFTASVVYQTYVPELTISATSQESATKNHVISSFSDLGVTLDLPSSNLRFFLVRGSPYITASVTKPTTLSINTSSAIESLSASSHRNTKYILKLKSKQTWIIYSSSPISLTNEGTEIRSNSYSGIIRFASLRNPHYESTLDKFSSSYPVSGDAEIKKPFHLRYKWQKKGNGGLLMLAHPLHVKLLPRLFSHVIVLRDFKYPSVDGDLVGVVGDSWELETKPVPVTWRSVKSVKKESYQEIVKALVKDVNELNSSNVTSTSSYFYGKLVARAARLALIADEVNNHEVIPKISIFLKETIQPWLDGSFKGNAFLYEKRWGGLVTRQGSVDKGADFGFGVYNDHHYHLGYFLYGIAVLAKIDTAWAKKYKSQTYALVTDFLNTDQRLKQSPRLRNFDLYMLHSWASGLTEFGDGRNQESTSEAVNAYYAAALVGLAYGDKRLISTASTLTALEIRAAQTWWHVKSKNKVYAEEFAKGNKIVGVLWSIKRDSGLWWAAAERKECRLSIQVLPLSPITESLFSDPSYVKELVEWTLPSVESKQNVEGWKGFIYALQGIYDKGKSLEKIRTLKGVDDGNSFTNLLWWIHSR

SEQ ID NO: 70 - protein, 656 aa, Vigna unguiculata
MSSSSSFMFPQTQSTVLPDPSTYFSSNLLSSPLPTNSFFQNFVLSKGSQPEYIHPYLIQTSKSSLSASYPLLFFTAAVLYQTFVPDLTISSSQTLPTQQNHVVSSFSDLGVTVDIPSSNLRFFLSRGSPFITASVTSSTSLSITTLHTILSLSPSNDKNTKYTLKLNNTQTWLIYASSPIYLNRDGASQVTSKPFSGIIRVAALPDDNPNNVAILDKFSSSYPSSGNATLHDPFRLVYQWQKEGSGDLLMLAHPLHAKLLSHNNTGNVNILRDFKYRSIDGDLVGVVGDSWKLEMNPIPVTWHSNKGVGKESYNEIVSALSKDVQTLNSPISTPSSYAIGKLIGRAARLALIAEEVSFPNVVPTIKEFLKRNIQPWLDGTVQGNGFLYEKKWGGLVTKMGSTDSSADFGFGVYNDHHYHLGYFLYGIAVLAKIDNEWGQKYKPQVYALLSDFMNLEQQNAHYPRLRCFDLYRLHSWASGVTEFADGRNQESTSEAVNAYYSAALVGVAYGDKSLVSAGSTLLAMEILGTQTWWHVKAEDKLYNEEFAKNNKIVGVLWSNKRDSGLWWAPATCRECRLGIQVLPLSPITETLFSDAGYVKGLVEWTLPSLSSEAWKGMTYALQGVYDKQTALQNIRRLKGFDDGNSFTNLLWWIHSR

SEQ ID NO: 71 - protein, 657 aa, Vigna unguiculata
MSSSSSFLFPQTQSTVLPDPSTYFSSNLLSSPLPTNSFFQNYVIPNGSQPEYIHPYLITTSNSSLSASYPFLLFTTALLYQAFVPDLTISSTQTQSHDQRNRVISSFSDLGITLDIPSSNLSFFLSRGSPFITASVTSSTSLSITTLHTILSLSPSNDNNTKYTLKLNNTQTWLIYASDPIYLNRDGASEVTSKPFSGIIRVAVLPDPNYATTLDKFSSCYPLSGDATLKESFRLVYQWEKEGSGDLLMLAHPLHVKLLSNKSNGQVTVLSDFKYRSIDGDLVGVVGDSWVLETDRIPVTWYSSKGVEKDSYDEIVSALVKDVEKLNSSAIGTSSSYFYGKRVGRAARLALIAEEVSFSKVVPTIMDFLKEAIEPWLDGTFVGNGFLYENKWSGLVTKLGSTDSTADFGFGVYNDHHYHLGYFLYGIAVLAKIDPEWGQKYKPQVYSLVTDFMNLGQRYSRIYPRLRCFDLYMLHSWAAGVTEFEDGRNQESTSEAVNAYYSAALVGLAYGDSNLVETGSTLVALEILAAQTWWHVKVEDNLYNEEFAKDNRIVGILWANKRDSKLWWASAECRECRLGIQVLPLLPITETLFSDADYVKELVEWTLPSLSSEGWKGMTYALQGIYDKETALQNIRTLTGFDDGNSYSNLLWWIHSR

SEQ ID NO: 72 - protein, 656 aa, Vigna unguiculata
MSPSFLFPQTQSTVLPDPSTYFSPNLLSSPFPTNSFFQNFVIPNGTQPEYFHPYHIQASNSSLSASYPFLFFTAAVLYQVFVPDLTISASQTTSYGQNRVISSYSDLGVTLDIPSSNLRFFLVRGSPFITASVTKPTSLSIKTVHTILSLSSYDGNTKFIIQLNNTQTWLIYTSSPIYLNHVPSEVTSKPFSGIIRIAALPDSNPSNVATLDKFSSCYPVSGDATLGKPFRLEYKWQKKRSGDLLMLAHPLHAKLLSRDCNVTVLHDFKYRSVDGDLVGVVGDSWVLETDPIPVTWHSKKGISKESFGEIVSALYKDVKGLNSSAITTNSSYFYGKLVGRAARLALIAEEVSYYKVIPKIRKFLKETIEPWLDGTFKGNGFLYERKWRGLVTEQGSTDSTADFGFGIYNDHHFHLGYFLYGIAVLAKIDPAWGKKFKPQAYSLATDFMNLGQRYNSDYPRLRCFDLYKLHSWASGLTEFEDGRNQESTSEAVNAYYAAALMGLAYGDSRLVDTGSTLLALEIRATQTWWHVKAEDNLYEEEFAKDNRIVGILWANKRDSKLWWATAECRECRLSIQVLPLLPVTETLFSDTVYTKELVEWTLPSLKNKTNVEGWKGFTYALQGIYDKSTALKQIRRLTGFDDGNSFSNLLWWIHSR

SEQ ID NO: 73 - protein, 655 aa, Phaseolus vulgaris
MSSSSSFLFPQTQSTVLPDPSTYFSSNLLSSPLPTNSFFQNYVIPNGSQPEYIHPYLIKSTNSSLSASYPLLLFTTALLYQAFVPDLTISSTQTHSQQQNRVISSFSDLGVTLDIPSSNLRFFLSRGSPFITASVTSSTSLSITTLYTILSLSSNNENNTKYTLKLNNTQTWLIYTSSPIHFNHNASEATSKPFSGIIRVAVLPNPNYETILDKYSSCYPLLGDATLEEPSRVVYQWQKEGSGDLLMLAHPLHVKLLSNNNNGNVTLLSDFKYRSIDGDLVGVVGDSWILQTDRIPVTWYSNNGVETNSYDEIVSALVKDVQALNSSAIGTTSSYFYGKRVGRAARLALIAEEVSFSKVVPTVTDFLKEAIEPWLDGTFEGNGFLYENKWGGLVTKLGSTDSSADFGFGVYNDHHYHLGYFLYGIAVLAKIDPEWGQKYKPQVYSLVTDFMNLGQRYNRNYPRLRCFDLYTLHSWAAGVTEFEDGRNQESTSEAVNAYYSAALVGLAYGDSSLVATGSTLVALEILAAQTWWHVKVEDNLYEEEFAKDNRIVGIVWANKRDSKLWWAGADCRECRLGIQVLPLLPITETLFSDADYVKELVEWTFPSLSSEGWKGMTYALQGVYDKQTALQNIRTLKGFDDGNSYSNLLWWIHSR

SEQ ID NO: 74 - protein, 655 aa, Phaseolus vulgaris
MSFSSSFLFPKTQSTVLPDPSTYFSSNLVSSPLPTNSFFQNFVLLNGSQPEYIHPYLIQTSKSSLSASYPLLFFTAAVLYQTFVPDLTISSTQTLPNEQNHVISSHSDLGVTLDIPSSNLRFFLSRGCPFITASVTSSTSLSIRTLHTILSLSSNNENNTKYTLKLNNTQTWLIYTSSPIHFNHNALEVTSKPFSGIIRVAVLPNPNYETILDKYSSCYPLLGDATLEEPSRVVYQWQKEGSGDLLMLAHPLHVKLLSNNNTGTDTILHNFKYSSIDGDLVGVVGDSWKLEMNHIPVTWHSNKGVEKESYDEIVSALSKDVQALNSSPIATASSYLYGKLIGRAARLALIAEEVSFPNVVPTIKEFLKKNIEPWLDGTFQGNGFLYENKWGGLVTKLGSTDSSADFGFGVYNDHHYHLGYFLYGIAVLAKIDPEWGQKYKPQVYSLLSDFMNLDHQHNAYYPRLRCFDLYMLHSWASGLKEFADGRNQESTSEAVNAYYSAALVGLAYGDSSLVATGSTLVALEILAAQTWWHVKVGEKLYKEEFAKDNRIVGVLWANKRDSGLWWASAECRECRLGIQVLPLLPITETLFSDADYVKELVEWTLPSLSSEGWKGMTYALQGVYDKQTALQNIRTLKGFDDGNSYTNLLWWIHSR

SEQ ID NO: 75 - protein, 667 aa, Phaseolus vulgaris
MSQFPFSSSQTMSSSFLFPQTQSTVLPDPSTYFSPNLLSSPLPTNSFFQNFVIPNGTVPEYFHPYHIQSSNSSLSASYPFLFFTAAVLYQVFVPDLTISASQTYSNAQNRVISSYSDLGVTLDIPTSNLRFFLVRGSPFITASVTKPTSLSITTVHTILSLSSYDDNTKFILQLNNTQTWLIYTSSPIYLNHAASQVSSKPFSGIIRIAALPDSNPNNVATLDKFSSCYPVSGDAALKKPFRVEYKWQRKRSGDLLMLAHPLHAKLLSHDCNVTVLHDFKYRSVDGDLVGVVGDSWVLETDPIPVTWHSKKGIDKESFGEIISALNKDVKELNSSAITTQSSYFYGKLVGRAARLALIAEEVSYPKVIPKIRNFLKETIEPWLDGTFKGNAFLYERKWRGLVTKQGSTDSTADFGFGVYNDHHFHLGYFIYGIAVLAKIDPAWGKQYKPQAYSLVTDFMNLGQRYNTDYPRLRCFDLYKLHSWASGLTEFEDGRNQESTSEAVNAYYAAALMGLAYGDSRLVDTGSTLLALEIRATQTWWHVKVEDNLYEEEFAKDNRIVGILWANKRDSKLWWAPAECRECRLSIQVLPLLPVTETLFFDTVYAKELVEWTLPSLKNKTNVEGWKGFTYALQGIYDKTTALKKIRMLTGFDDGNSFSNLLWWIHSR

SEQ ID NO: 76 - protein, 652 aa, Glycine max
MSSFLFPQTQSTVLSDPSTYFSSNLLSSPLPTNSFFQNFVIPNGSQAEYIHPYLIKTSNSSLSASYPLLILFTTAVLYQTFVADITISSTQTTSQNHVISSYSDLGVTLDIPSSNLRFFLSRGSPFLTVSVTSPTSLSITTVHTIVSLSSNDDNNTKYTLKLNNTQTWLIYTSSPIYFTHNNASEVTSKPFSGIIRVAVLPNHNYVTILDKFSTCYPLSGNATLVEPFRVVYEWQKEGSGDLLMLAHPLHVKLLSNNYNGLVTVLNDFKYRSIDGDLVGVVGDSWVLETNPIPVTWYSNKGMEKDSYDEIVSALVKDVQELNSSSIGTSSSYFYGKRVGRAARLALIAEEVSFSNVVPTIKKFLKESIEPWLDGTLQGNGFLYENKWGGLVTKLGSTDSTADFGFGVYNDHHYHLGYFLYGIAVLAKIDPEWGQKYNPQVYSLVTDFMNLGQKYNSRYPRLRCFDLYNLHSWASGVTEFADGRNQESTSEAVNAYYSAALVGLAYGDSNLVAIGSTLLALEILAAQTWWHVKAEGNLYEEEFAKENKIVGVLWANKRDSALWWGPATCRECRLGIQVLPLSPVTETLFSDADYVKELVEWTMPSLTSEGWKGMTYALQGIYDKETALENIRKLKGFDDGNSLSNLLWWIHSR

SEQ ID NO: 77 - protein, 631 aa, Glycine max
LPNPSTYFSSNLVSSPLPTNSFFQNFALQNGTQAEYIHPYLIKTSNFSLSASCPLLLFTTAVLYQTFVADITISSTQTTSQNHVISSYSDLGVTLDIPSSNLRGSPYITASVTKPTSLSITTVRSIVSLCSNNKENTKYTLKLNNTQTWLIYTSSPIYLNHDAASNITSKHFLQCCLFPTSRVWQFSTSSALVTRSLSGNATLVKPFRVTYEWQKKGPGFLLTLAHPLHVKLLQYKKNHRMIVLRDFKYRSIDGDLVGVVGDSWLLKTDTIPVTWHSNKGVEKESHDEIVSALSKDVEALSSSPIATESSYYYGKLIGRAARLALIAEEVSSPNVIPTIQKFLKDSIEPWLDGTFQGNGFLYENKWGGLVTKQGSTDSGADFGFGVYNDHHYHLGYFLYGIAVLAKVDLQWGQKYKPQVYSLVSDFMNSGQKYNSHYPRLRCFDLYKLHSWTSGVTEFTDGRNQESTSEAVNAYYSAALVGLAYDDSNLVATGSTLLALEILAAQTWWHVKAEGNLYEEEFAKENKIVDALWANKRDSALWWAPATCRECRLGIEVLPLSPVTETLFYDADYVKELVEWTMPSLTSEGWKGMTYALQGIYDKETVLQNIRMLTGFDDGNSFTNLLWWIHSR

SEQ ID NO: 78 - protein, 653 aa, Glycine max
MSSNFLFPQTQSTVLPNPSTYFSSNLLSSPLPTNSFFQNFVIPNGSQAEYIHPYLVKTSNSSLSASYPLLLFTTALLYQSFVPDITISSTQTHSNQQNREISSYSDLSVTLDIPSSNLRFFLSRGSPFITASVTSPTSLSITTVHTIVSLSSNDDNNTKYTLKLNNTQIWLIYTSSPIYLNHDGASNITSKPFSGIIRVAALPDSNSKSVAILDKFSSCYPLSGNATLVEPFRVVYQWQKESSGDLLMLAHPLHVKLLSNSQVTVLKDFKYRSIDGDLVGVVGDSWVLETDPIPVTWYSNKGVDKDSYDEVVSALVKDVQELNSSAIGTSSSYFYGKRVGRAARLALIAEEVSFSNVVPTIKKFLKESIEPWLDGTFQGNSFLYENKWGGLVTKQGSTDSTADFGFGVYNDHHYHLGYFLYGIAVLAKIDPQWGQKYKPQVYSLVTDFMNLGQRYNRFYPRLRCFDLYKLHSWAAGLTEFEDGRNQESTSEAVNAYYSAALVGLAYGDSSLVDTGSTLVALEILAAQTWWHVKVEDNLYEEEFAKDNKIVGVLWANKRDSKLWWASAECRECRLGIQVLPLLPITETLFSDADYVKELVEWTVPFLSSQGWKGMTYALQGIYDKETALENIRKLKGFDDGNSLSNLLWWIHSR

SEQ ID NO: 79 - protein, 657 aa, Glycine max
MSSSFLFPQTQSTVLPDPSTYFSPNLLSSPLPTNSFFQNFVIPNGTQPEYIHPYLIKTSNSSLSASYPLLFFTTAVLYQAFVPDITISSPQTHSRQQNRVISSYSDLGVTLDIPSSNLRFFLSRGSPFITASVTKPTSLSITTVHTIVSLSANDDKNTKYTLKLNNTQAWLIYTSSPIYLNHDAASNVTSKPFSGIIRVAVLPDSNSKCVKILDKFSSCYPLSGNATLEKPFRVVYEWLKEGSGNLLMLAHPLHVKILSSTNNGQVNVLRHFKYRSIDGDLVGVVGDSWVMETNPIPVTWYSNKGVEKESYDEIVSALVTDVQGLNSSAIETIISSYFYGKRVGRAARFALIAEEVSFPKVIPSVKKFLKETIEPWLDGTFPGNGFQYENKWGGLVTKLGSTDSTADFGFGIYNDHHYHLGNFLYGIAVLAKIDPQWGQKYKPQVYSLVTDFMNLGPSYNRFYPRLRNFDLYKLHSWAAGLTEFEHGRNQESTSEAVTAYYSAALVGLAYGDSSLVATGSTLMALEILAAQTWWHVKEKDNLYEEEFAKENRVVGILWANKRDSKLWWARAECRECRLGIQVLPLLPITETLFSDADYAKELVEWTLPSARREGWKGMTYALQGIYDRKTALQNIRMLKGFDDGNSFTNLLWWIHSR

SEQ ID NO: 80 - protein, 655 aa, Glycine max
MSSPSSFLFPQTQSTVIPDPSTYFSPNLLSSPLPTNSFFQNFVIPNGTQPEYIHPYLIQSSNSSLSASYPLLLFTTALLYQAFVPDLTISATKRYSSYQQNRVISSYSDLGVTLDIPSSNLRFFLVRGSPYITASVTKPTPLSIKTVHTIVSLSSDDSNTKHTLKLNNTQTWIIYTSSPIYLNHVPSEVTSKPFSGIIRIAALPDSGSKYVATLDKFSSSYPVSGDAALKKPFRLEYKWQKKRSGDLLMLAHPLHVKLLSYDRDVTVLNDFKYRSIDGDLVGVVGDSWVLETNAIPVTWYSNKGVDKESYGEIVSALVKDVRALNSSAIGTNSSYFYGKQVGRAARLVLIAEEVSYPKVIPKVKKFLKETIEPWLDGTFKGNGFLYERKWRGLVTKQGSTDSTADFGFGIYNDHHFHLGYFIYGIAVLAKIDPQWGQKYKPQVYSLVTDFMNLGQRYNSDYTRLRCFDLYKLHSWAAGLTEFEDGRNQESTSEAVNAYYAAALMGLAYGDSSLVATGSTLVALEILAAQTWWHVKAEDNLYEEEFAKDNRIVGILWANKRDSKLWWASAECRECRLGIQVLPLLPITETLFSDADYVKELVEWTVPFLSSQGWKGMTYALQGIYDRETALQNIRKLTGFDDGNSFTNLLWWIHSR

SEQ ID NO: 81 - protein, 653 aa, Glycine max
MSSSFLFPQTQSTVIPDPSTYFSPNLLSSPLPTNSFFQNFVIPNGTQPEYIHPYLIQSSNSSLSASFPLLLFTTALLYQAFVPDLTISASKTYSSYQQNRVVSSYSDLGVTLDIPSSNLRFFLVRGSPYITASVTKPTPLSIKTVHTVVSLSSDDYNTKHTLKLNNSQAWIIYTSSPIYLNHVPSEVTSKPFSGIIRIAALPDSDSKYVETLDKFSSCYPVSGDAALKKPFSVEYKWQKKRSGDLLMLAHPLHAKLLSYDRDVTVLNDFKYRSIDGDLVGVVGDSWVLETNPIPVTWNSNKGVEKESYGEIVTALVKDVQALNSSAIGTNSSYFYGKQVGRAARLALIAEEVSYPKVIPKVKKFLKETIEPWLDGTFKGNAFLYERKWRGLVTKHGSTDSTADFGFGIYNDHHFHLGYFIYGIAVLAKIDPQWGQKYKPQVYSLVTDFMNLGQRYNSDYTRLRCFDLYKLHSWAAGLTEFEDGRNQESTSEAVNAYYAAALLGLAYGDSSLVDTGSTLVALEILAAQTWWHVKAEDNLYEEEFAKDNRIVGVLWANKRDSKLWWAPATCRECRLGIQVLPLLPITETLFSDADYVKELVEWTVPFLSSQGWKGMTYALQGIYDKKTALQNIRKLTGFDDGNSFTNLLWWIHSR

SEQ ID NO: 82 - protein, 656 aa, Cajanus cajan
MSPSFLFPQTQSTVLPDPSTYFSPNLLSSPLPTNSFFQNFVIPNGSQPEYIHPYLIKSSNTSLSASYPFLFFTAAILYQVFVPDLTISASRTYSNKQNRVVSSYSDLGVTLDIPSSNLRFFLVRGSPFITASVTKPTSLSITTNQTIVSLTSTNDNTKHTLQLNNTQTWLIYTSSPIYLNHVPSEVTSKPFSGIIRIAALPDSNPKNVEILDKFSSCYPVSGDATLKKPFRVVYKWQKKQSGDLLMLAHPLHAKLLSYDREVTVLHDFKYRSVDGDLIGVVGDSWVLETDPIPVTWHSNKGIKKESYGEIVSALVKDVKELNSSAITTNSSYFYGKLVGRAARLALIAEEVSFPKVIPKVRKFLKETIEPWLDGTFKGNGFLYESKWRGLVTEQGSTDSTADFGFGIYNDHHFHLGYFLYGIAVLAKIDPVWGQKYKSQAYSLVTDFMNLDQRYNSDYPRLRNFDLYKLHSWASGVTEFEDGRNQESTSEAVNAYYAAGLMGLAYRDTDLVATGSTLLALEIRAAQTWWHVKVGDNLYEEDFAKDNRIVGVLWANKRDSKLWWAPAECRECRLSIQVLPLLPVTETLFSDAVYAKELVEWTLPSLKNKTNVEGWKGFTYALQGIYDKNTALKKIRMLKGFDDGNSFSNLLWWIHSR

SEQ ID NO: 83 - protein, 656 aa, Cajanus cajan
MSSPFVFPETQSTVLPDPSTYFSPNLLSSPLPTSSFFQNFVIPNGSQPEYIHPYLIKTSNTSLSASYPLLIFTAAVLYQAFVPDLTISSTQTQTKEQNRVVSSHSDLGVTLDIPSSNLRFFLSRGSPFITASVTSPTSLSITTNHTIASLSSNDNKTKHTLRLNNTQTWLIYTSSPINLNHDDGASEVTSKPFYGTIRLAVLPDSKYEATLDKFSSSYPLSGDATFENSKPFRLVYQWQKKGSENLLMLAHPLHVKLLSKYNNAGVTVLHDFKYRSIDGDLVGVVGDSWVLEMDPIPVTWYSNKGVNDGSRDEIVSALVKDVEALNSSAITTKSSYFYGKQVGRAARLALIAEEVSFSKVVPTIKKFLKETIDPWLDGTFKGNGFLYEKKWGGLVTKLGSTDSTADFGFGVYNDHHFHLGYFLYGIAVLAKIDPEWGQKYKPQAYSLVTDFMNLDQKYSTIYPRLRCFDLYKLHSWASGVTEFEDGRNQESTSEAVNAYYSAALVGLAYDDSSLVATGSTLVALEILAAQTWWHVKVGENLYQEEFAQDNRIVGILWANKRDSKLWWATAECRECRLGIQVLPLLPITETLFSDAVYVKELVEWTMPYLSNEGWKGMTYALQGIYDKETALDEIRKLKGFDDGNSYTNLLWWIHSR

SEQ ID NO: 84 - protein, 656 aa, Phaseolus lunatus
MSSSFLFPQTQSTVIPDPSTYFSPNLLSSPLPTNSFFQNFVIPNGTLPEYFHPYHIQSSNSSLSASYPFLFFTAAVLYQVFVPDLTISASQTYSHGQNRVISSYSDLGVTLDIPTSNLRFFLVRGSPFITASVTKPTSLSITTVHTILSLSSYNDNTKFILQLNNTQTWLIYTSSPIYLNHAASEVTSKPFSGIIRIAALPDSDPNNVATLDKFSSCYPVSGDAALKKPFRVEYKWQRKRSGDLLMLAHPLHAKLLSHDCNVTVLHDFKYRSVDGDLVGVVGDSWVLETDPIPVTWHSKKGINKESFGEIVSALNKDVKELNSSAITTQSSYFYGKLVGRAARLALIAEEVSYPKVIPKIIKFLKETIEPWLDGTFKGNAFLYERKWRGLVTKQGSTDSTADFGFGVYNDHHFHLGYFVYGIAVLAKIDPAWGKKYKPQAYSLVTDFMNLGQRYNSDYPRLRCFDLYKLHSWASGLTEFEDGRNQESTSEAVNAYYAAALMGLAYGDSRLIDTGSTLLALEIRATQTWWHVKAEDNLYEEEFAKDNRIVGILWANKRDSKLWWAPAECRECRLSIQVLPLLPVTETLFFDTVYAKELVEWTLPSLKNKTNVEGWKGFTYALQGIYDKTTALKKIRMLTGFDDGNSFSNLLWWIHSR

SEQ ID NO: 85 - protein, 655 aa, Phaseolus lunatus
MSSSSSFLFPQTQSTVLPDPSTYFSSNLLSSPLPTNSFFQNYVIPNGSQPEYIHPYLIKTTNSSLSASYPFLLFTTAVLYQAFVPDLTISSTQTHSHQQNRVISSFSDLGVILDIPSSNLRFFLSRGSPFITASVTSSTSLSITTLHTILSLSSNDDDNTRYTLKLNNSQTWLIYTSSPIHLNHNASEVTSKPFSGIIRVAVLPNPNYETILDKYSSSYPLLGDATLEEPSRVVYQWQKEGSGDLLMLAHPLHVKLLSNNNDGNVTLLSDFKYRSIDGDLVGVVGDSWILQTDRIPVTWYSNNGVETNSYEEIVSALVKDVQALNSSAIGTNSSYFYGKRVGRAARLALIAEEVSFSKVVPTVTDFLKGAIEPWLDGTFEGNGFLYENKWGGLVTKLGSTDSSADFGFGVYNDHHYHLGYFLYGIAVLAKIDTEWGQKYKPQVYSLVTDFMNLGQRYNRIYPRLRCFDLYMLHSWAAGVTEFEDGRNQESTSEAVNAYYSAALVGLAYGDSSLVATGSTLVALEILAAQTWWHVKVEDNLYEEEFAKDNRIVGIVWANKRDSNLWWAGADCRECRLGIQVLPLLPITETLFSDSDYVKELVEWTLPSLSSEGWKGMTYALQGIYDKQTALQNIRTLKGFDDGNSYSNLLWWIHSR

SEQ ID NO: 86 - protein, 731 aa, Phaseolus lunatus
MLKKLRRKVSTALRSGLKNGSKPYKNPSPPPLSPLSLPLPLPLEPVRTMSHTTKHSPFLFPHANSSVVPDPSNFFSPNLLSNPLPTNSFFQNFTLKNGDQPEYIHPYLIKSSNFSLSLSYPSRFFNSSFTYQVFNPDLTISSSQKPHLSHFNHTISSHNDLSVTLDIPSSNLRFFLVRGSPFLTLSVTQPTPLSITTIHAILSFSSSDSLTKHTFNLNNGQTWILYASSPIRLSHGLSEINSDAFSGIIRIALLPDSDSKHEAVLDRFSSCYPVSGEAVFARPFCVDYKWEKKGWGDLLMLAHPLHLQLLADGGCGDVNVLSDFKYGSIDGDLVGVVGDSWSLKTDPVSVTWHSIRGVREESRDEVVSALVNDVEGLNSSSITTNSSYFYGKLIARAARLALIAEEMCFLDVIPKVRKYLKETIEPWLEGTFNGNGFLYDRKWGGIVTKQGSNDAGADFGFGIYNDHHYHLGYFVYGIAVLAKIDPVWGRKYKPQAYSLMADFMTLSRRSNSNYTRLRCFDLYKLHSWAGGLTEFADGRNQESTSEAVNAYYSAALMGLAYGDTHLVATGSTLTALEIHAAQMWWHVKQGDNHYGEGFEKENKVVGVLWANKRDSGLWFAPPEWKECRLGIQLLPLLPISEVLFSDVDFVKDLVEWTLPALNREGVGEGWKGFVYALQGIYDNEGALQRVRSLNGFDDGNTLTNLLWWIHSRSDEEEFGHGKHCWFGHYCH

SEQ ID NO: 87 - protein, 739 aa, Phaseolus lunatus
MFKKLGRKIEREITKPFKNKPRPRTSSPPPPPPPPPPPPPSSTPPPPPPPPSPPPLPKQPNAPFLFPQAHSTILPDPSTFFAPNLLPSPLPTNSFFQNYVLQNGDTPEYIHPYLIKSSNSSLSLSYPSLNFNSSFIAQVFNPDITISSTDSKTTPGLHASHVISSFSDLSVTLDIPSSNLRFFLVRGSPFVTASVTCPTPLSITTMHAILSLSSNNSLTKHTLQLNNGQSWLINTSSPISLNHSLSEITSGEFSGIIRIAVLPDSDPKYEVILNRFSSCYPVSGDATFTNPFCVKYKWEKKGWGELLMLAHPLHLQLLNDGDSGVTVLHNLKFRSIDGELVGVVGDSWLLKTDPVSVTWHSTRGIKEEFHEEIYSVLSEDVEALNPKGITTTSCYFYGKIIARAARLALIAEEVAFLDAMPVIRKFLKEIIEPWLDGTFSGNGFLYEGKWGGIVTKQGSKDSEADFGFGVYNDHHYNLGYFLYGIAVLAKIDPAWGRKYKPQAYSLVADFMSLARRSDSNYTRLRCFDLYKLHSWAGGLTEFADGRNQESTSEAVNAYYSAALTGLAYGDTQLIATGSTLAALEIHAAQMWWHLGEGNKLYEEDFTKDNKVVSVLWANKRDSGLWFAPSQWRECRLGIHVLPLSPITEALFSDVDYVKELVEWTVPNLNRKCVGEGWKGFIYALEGTYDKESALQKVRSLKVFDDGNSMSNLLWWIHSRGDVEEEFGQGKQCWFGHYCH

SEQ ID NO: 88 - protein, 662 aa, Phaseolus lunatus
MVKQNKTHFIFPETQSTVLPDPSNFFSSTLLSKPLPTNSFFQNFVLKNGDQPEYIHPYLIKSSNSSLSLSYPSRQVSSAVIYQVFNADLTISSKQSSSGKHLISSYSDLSVTLDIPSSNLSFLLVRGSPFLTVSVTQPTPLSITTIHTILSFSSNETNTKYTFQFNNGQTWILYASSSIKLSHTLSEITSDTFSGIVRIALLPDSDSKHEAVLDKFSSCYPVSGEAIFREPFCVEYKWEKKGSGDLLLLAHPLHVQLLSNGDNDVTVLEDFKYGSIDGDVVGVVGDSWVLQTDPVYVTWHSTKGVKEESHDEIVSALSNDVDGLNSSSISTTSSYFYGKLIARAARLALIAEELSYPDVIPKVKKFLKETIEPWLVGTFNGNGFLHDKKWGGIITQQGSNDGGGDFGFGIYNDHHYHLGYFLYAIAVLVKLDPAWGRKYKAQAYSIVQDFMNLDTKLNSNYTRLRCFDLYVLHSWAGGLTEFSDGRNQESTSEAVCAYYSAALMGLAYGDAHLVSLGSTLTALEILGTKMWWHVEEEGKLYEEEFTRENRIMGVLWSNKRDTGLWFAPAEWKECRLGIQLLPLVPISEAIFSNAEYVKQLVEWTLPALNRDGVGEGWKGFVYALEGIYDNESALQKIRNLTGFDGGNSLSNLLWWIHSIGNE

SEQ ID NO: 89 - protein, 656 aa, Phaseolus acutifolius
MSSSFLFPQTQSTVLPDPSTYFSPNLLSSPLPTNSFFQNFVIPNGTVPEYFHPYHIQSSNSSLSASYPFLFFTAAVLYQVFVPDLTISASQTYSNAQNRVISSYSDLGVTLDIPTSNLRFFLVRGSPFITASVTKPTSLSITTVHTILSLSSYDDNTKFILQLNNTQTWLIYTSSPIYLNHAASQVTSKPFSGIIRIAALPDSNPNNVATLDKFSSCYPVSGDAALKKPFRVEYKWQRKRSGDLLMLAHPLHAKLLAHDCNVTVLHDFKYRSVDGDLVGVVGDSWVLETDPIPVTWHSKKGIDKESFGEIVSALNKDVKELNSSAITTQSSYFYGKLVGRAARLALIAEEVSYPKVIPKITKFLKETIEPWLDGTFKGNAFLYERKWRGLVTKQGSTDSTADFGFGVYNDHHFHLGYFIYGIAVLAKIDPAWGKQYKPQAYSLVTDFMNLGQRYNSDYPRLRCFDLYKLHSWASGLTEFEDGRNQESTSEAVNAYYAAALMGLAYGDSRLVDTGSTLLALEIRATQTWWHVKVEDNLYEEEFAKDNRIVGILWANKRDSKLWWAPAECRECRLSIQVLPLLPVTETLFFDSVYAKELVEWTLPSLKNKTNVEGWKGFTYALQGIYDKTTALKKIRMLTGFDDGNSFSNLLWWIHSR

SEQ ID NO: 90 - protein, 655 aa, Phaseolus acutifolius
MSFSSSFLFPKTQSIVLPDPSTYFSSNLVSSPLPTNSFFQNFVLLNGSQPEYIHPYLIQTSKSSLSASYPLLFFTAAVLYQTFVPDLTISSTQTLSNEQNHVISSHSDLGVTLDIPSSNLRFFLSRGSPFITASVTSSTSLSITTLHTILSFSSNNENNTKYTLKLNNTQTWLIYTSSPIHFNHNASEVTSKPFSGIIRVAVLPNPNYETILDKYSSCYPLLGDATLEEPSRVVYQWQTEGSGDLLMLAHPLHVKLLSNNNTGTVTILHDFKYSSIDGDLVGVVGDSWKLEMNHIPVTWHSNKGVEKESYDEIVSALSKDVQALNSTPIATASSYLYGKLIGRAARLALIAEEVSFPNVVPTIKEFLKENIEPWLDGTFQGNGFLYENKWGGLVTKLGSTDSSADFGFGVYNDHHYHLGYFLYGIAVLAKIDLEWGQKYKPQVYSLLSDFMNLDHQHNAYYPRLRCFDLYMLHSWASGLKEFADGRNQESTSEAVNAYYSAALVGLAYGDSSLVATGSTLVALEILAAQTWWHVKVGEKLYKEDFAKDNRIVGVLWANKRDSGLWWASAECRECRLGIQVLPLLPITETLFSDADYVKELVEWTLPSLSSEGWKGMTYALQGVYDKQTALQNIRTLKGFDDGNSYSNLLWWIHSR

SEQ ID NO: 91 - protein, 655 aa, Phaseolus acutifolius
MSSSSSFLFPQTQSTVLPDPSTYFSSNLLSSPLPTNSFFQNYVIPNGSQPEYIHPYLIKTTNSSLSASYPLLLFTTALLYQAFVPDLTISSTQTHSHQQNRVISSFSDLGVTLDIPSSNLRFFLSRGSPFITASVSSSTSLSITTLHTILSLSSNNDNNTKYTLKLNNTQTWLIYTSSPIHFNHNASEVTSKPFSGIIRVAVLPNPNYETILDKHSSCYPLLGDATLEEPSRVVYQWQKEGSGDLLMLAHPLHVKLLSNNNNGNVTLLSDFKYRSIDGDLVGVVGDSWILQTDRIPVTWYSNNGVEKNSYDEIVSALVKDVQALNSSAIGTSSSYFYGKRVGRAARLALIAEEVSFSQVVPTVTDFLKKAIEPWLDGTFEGNGFLYENKWGGLVTKLGSTDSSADFGFGVYNDHHYHLGYFLYGIAVLAKIDPEWGQKYKPQVYSLVTDFMNLGQRYNRNYPRLRCFDLYMLHSWAAGVTEFEDGRNQESTSEAVNAYYSAALVGLAYGDSSLVATGSTLVALEILAAQTWWHVKVEDNLYEEEFAKDNRIVGIVWANKRDSKLWWAGADCRECRLGIQVLPLLPITETLFSDSDYVKELVEWTFPSLSNEGWKGMTYALQGVYDKQTALQNIRTLKGFDDGNSYSNLLWWIHSR

SEQ ID NO: 92 - protein, 741 aa, Phaseolus acutifolius
MFKKLGRKIEREITKPFKNKPRPRPSSPPPPPPPPPPPLPSSTPPPPPPPPSPPPPLPKQPNAPFLFPQAHSTILPDPSTFFAPNLLSSPLPTNSFFQNYVLQNGDTPEYIHPYLIKSSNSSLSLSYPSLNFNSSFIAQVFNPDITISSTESKTTPGLHARHVISSFSDLSLTLDIPSSNLRFFLVRGSPFVTASVTCPTPLSITTMHAILSLSSNNSLTKHTLQLNNGQSWLINTSSPISLNYSLSEITSGEFSGIIRIAVLPDSDPKYEVILNRFSSCYPVSGDATFTNPFCVKYKWEKKGWGELLMLAHPLHLQLLNDGGDSGVTVLHNLKFRSIDGELVGVVGDSWLLKTDPVSVTWHSTRGIKEEFHEEIFSVLSEDVEALNPLGITTTACYFYGKIIARAARLALIAEEVAFLDAMPVVRKFLKEIIEPWLDGTFSGNGFLYEGKWGGIVTKQGSKDSGADFGFGVYNDHHYNLGYFLYGIAVLAKIDPAWGRKYKPQAYSLVADFMSLGRRSDSKYTRLRCFDLYKLHSWAGGLTEFADGRNQESTSEAVNAYYSAALMGLAYGDTQLIASGSTLAALEIHAAQMWWHLGEGHKLYEEDFTKENKVVSVVWANKRDSGLWFAPSQWRECRLGIHVLPLSPITEALFSDVGYVKELVEWTVPNLNRKCVGEGWKGFIYALEGTYDKESAVQKVRSLKVFDDGNSMSNLLWWIHSRGDVEEEFGQGKQCWFGHYCH

SEQ ID NO: 93 - protein, 710 aa, Phaseolus acutifolius
MLKKLRRKVSTALRSGLKNGSKPYKNPSPPPSSPLPLPLVPVRTMSHTRKHSPFLFPHVDSSVVPDPSNFFSPNLLSNPLPTNSFFQNFTLKNGDQPEYFHPYLVKSSNFSLSLSYPSRSFNSSFTYQVFNPDLTISSSQKPHLSHFNHTISSHNDLSVTLDIPSSNLRFFLVRGSPFLTLSVTQPTPLSITTIHAILSFSSSDSLTKHTFNLNNGQTWILYASSPIRLSHGLSEINCDAFSGIIRIALLPDSDSKHEAVLDRFSSCYPVSGEAVFARPFCVEYKWEKKGWGDLLMLAHPLHLQLLADGGCDVNVLSDFKYGSIDGDLVGVVGDSWSLKTDPVSVTWHSIRGVREESRDEVVSALVNDVERLNSSSITTNSSYFYGKLIARAARLALIAEEMCFLDVIPKVRKYLKETIEPWLEGTFNGNGFLYDRKWGGIVTKQGSNDAGADFGFGIYNDHHYHLGYFVYGIAVLAKIDPVWGRKYKPQAYSLMADFMTLSRRSNSNYTRLRCFDLYKLHSWAGGLTEFADGRNQESTSEAVNAYYSAALMGLAYGDTHLVATGSTLTALEIHAAQMWWHVKQGDNHYGEEFERENKVVGVLWANKRDSGLWFAPPEWKECRLGIQLLPLLPISEVLFSDVDFVKDLVEWTLPALNREGVGEGWKGFVYALQGIYDNEAEVMKRNLVMENTAGLVITATRQLLPDECPL

SEQ ID NO: 94 - protein, 662 aa, Phaseolus acutifolius
MVKQNKTHFIFPETQSTVLPDPSNFFSSTLLSKPLPTNSFFQNFVLKNGDQPEYIHPYLIKSSNSSLSLSYPSRQVSSAVIFQVFNADLTISSKQGSSGKHVISSYSDLSVTLDIPSSNLSFLLVRGSPFLTVSVTQPTPLSITTIHAILSFSSNKTNTKYTFHFNNGQTWILYSSSTIKLSHTLSEITSDAFSGIVRIALLPDSDSKHEAVLDKFSSCYPVSGEAIFREPFCVEYKWEKKGSGDLLLLAHPLHVQLLSNGDNDVTVLEDFKYGSIDGDVVGVVGDSWVLQTDPVYVTWHSTKGVKEESHDEIVSALSNDVEGLNSSSISTTSSYFYGKLIARAARLALIAEELSYPDVIPKVKKFLKESIEPWLEGTFNGNGFLHDKKWGGIITQQGSNDGGGDFGFGIYNDHHYHLGYFLYAIAVLVKLDPAWGRKYKAQAYSIVQDFMNLDTKLNNNYTRLRCFDLYVLHSWAGGLTEFSDGRNQESTSEAVCAYYSAALVGLAYGDAHLVSLGSTLTALEILGTKMWWHVEEEGSLYEEEFTRENRIMGVLWSNKRDTGLWFAPAEWKECRLGIQLLPLVPISEAIFSNAEYVKQLVEWTLPALNRDGVGEGWKGFVYALEGIYDNESALQKIRNLAGFDGGNSLSNLLWWIHSIGNE

SEQ ID NO: 95 - protein, 658 aa, Cicer arietinum
MSSSSVPFLFPQTHSTILPNPSNFFSPNLLSTPLPTNSFFQNFVLQNGDQPEYIHPYLIKSSNSSLSFSYPLLLFTTSFLYQVFVPDLTISSSQKTTSKNKHVISSYSHLSVTLEIPSSNLRFFLVRGSPFITANVTKPTSLSITTLNKIVSFSSFDYKKTKHTLQLNNTQKWIIYTSSPINFNHDGFEVISNPFSGIIRIAIVPNSNPFYEKTLDKFSSSYPVSGDANIKKNFSLVYNFQKKRLGDLLMLAHPLHVKLLSNDVKVLHDFKYKSVDGDLVGVVGDSWLLKNDPVSVNWYSNKGVAKESHNEIVSALIKDVNELNLSSISTTSSYFYGKIVGRAARFALIAEEVSYLKVIPKIKFFLKETIEPWLNGNFKGNGFLYEKKWGGLVTQQGLNDSSADFGFGVYNDHHYHLGYFLYGISVLVKIDPLWGQKYKPQVYSLLKDFMNLGERDNKNYPSLRCFDHYKLHSWASGLTEFENGRNQESSSEAVNAYYSAALIGLAYGDSKIVEIGSTILAFEIKAAQTWWHVKLENNLYGEDFAKENRIVGILWANKRDSKLWWAPSECRECRLSIQVLPLLPISETLFFDGVYAKELVEWTLPSLKNKTNVEGWKGFTYALEGIYDKEIALKNIRGLKGFDDGNSFTNLLWWIHSR

SEQ ID NO: 96 - protein, 673 aa, Cicer arietinum
MSTIKKNTPFIFPQTNSTVLPDPSNFFSPNLLSTPLPTNSFFQNFSLKNGDQPEYIHPYLIKSSNSSLSVSYPSHFSNSSFIYQVFNADLTITSLEQKTNQTSNEKHIISSYSDLSVTLDIPSSNLSFFLVRGSPYLTFSVTKPTPLSISTIHAIEFLVPTDPSITRYTFQLNNGQTWLLYASSPIKLSHDLSEITCEPFSGVIRIALLPNNDRKIEDVLEKYSSCYPLSGDAFLREPFCVEYKWQKNGSSDLLLLAHPLHVKLLSNSESDVTFLNDLKYTSIDGDLVGVVGDSWILKTEPVSITWHSSKGVKEESHDEIVSSLSKDVEGLNSSAITTTSSYFYGKLIARAARLALIAEEVFFFDAIQKVRNFLKETIEPWLEGTFNGNGFLYDRKWGGIITQQGSNDSNGDFGFGIYNDHHYHLGYFLYAIAVLVKIDPTWGRKYKTQAYSLMEDFMNLNIRLNSNYTRLRCFDLYKLHSWAGGLTEFSDGRNQESTSEAVNAYYAAALMGMAYGDASLVSIGSTLTSLEILGTKMWWHVKKEGKLYEEEFTKENRIMGVLWSNKRDSGLWFAAAESREARLGIQLIPLSPISEVLFSDVSYVKDLVEWTLPALNREGVGEGWKGFLYSLQGVYDNQGALEKIRNLNGFDGGNSLTNLLWWIHSRGEDGDDE

SEQ ID NO: 97 - DNA, 2789 bp, Medicago truncatula
  source 1..2789: mol_type = genomic DNA
gaaggagatttatagggggagagagtgaggaggaaaaaatgtgagatggtgtgaggaaaaaatgtgagatgtggttttggtttcaaaatgctcgtgaagctgaaattcctagcttctatgatagggaaaacatgattcaacctttaatttctcaaagtggtaggtactttacctaattacctataaatttaagccttcaatttctctttctcacaatttttctttgtttcgttacttcacatcacttcaaccttaaattttcttgtgaagattttgcatatccaagctttcgatttccttcagcttttcccttatttttgcaaatttcgtggtaatcccattactcatagtagatgtgttagttgggtgatattattgcatattactctatatttatatatgatgcttatgagctacttgtgagaaacaatccattactctgccaaagtctgaaggtgaatacatattcatatatgtttttctttgctttgattacttgtttcaatattgttaatttctatttctatttgctctaattgttacaagttttaccttcattttttcatcctacatcaatgcattgtacgtgattggaaccaataaaatccatgtatgataagttgaaataattatctaatgagaatgaaatttatttttaatacaattaaaaggatagtttggtcattacacattaaaattaattttgatttaaactatccaaacaacatcaattcataagattcaattatattaaagtgtatccaaacataaatcaattcacatccaactcacttttaaccaaaatcaattctctcaaactcaattttattaaaatctattctctcaaacttaattttaaccaaatacacacttaatagaacaaaaaaatataaaagatgaattggcatgaattttaagagcatgcatgaattcgcgtgacaaaaacatgaggaaacatttcttaaaataaggaaacatttttttgacctataaactacaactactccaactcaaactcacgagaatggttaaaacaccacccaaatttccacccaaaatatattttacccaattaataatttactggcgatacaatgaacaaagtaagattaattacaatcttatttttttgacaaagattaattactatctatttatgtgatttgaaattatgaagcccggatacggatacgatacggatacatgattttttttttaaatttaggatacgatacaactaggatacattaacaaatattttatatatgtatgtaaaatacttgaagaaaataatcattgattattcttcattattatattattataaaagtcacaattttaagaaaaaatacatgtaatcaaaataaaaaaatgaaaagtttttttatcaaccatattatattttcatatatgttaggaaattgagagaagcatcattaaaaacactcaacataaattttatttaatgttgattaactgaatcacctctctcgttaccttcattcacaagaagtataattttaagtgattcatcgagaaacaattttattgatgcatatctaaaattgaatcatatacatctctactaagttttcacaacattgtctcactttttttaccctgcatcttttttaagatatgtatatgtgaagtatcttaaaagtatttttgtgtatctctgcagtaacatataagtatctgataaaataatttttaacaattgaaataattaactccgatacgtcgtgggtacgtatccacgaatatccgtggagtatcgatatccgatacgggtacgtctccgttttggagtatgcgaggcttgatagaaaatttatatcttaccattaaaatttgtaaccaagcaagcaaattagttgattggaccccattgtttcttcgtgcatgattgatatttatttgtttatttcaaaatagtaaaaatcataattttgactaacatacagtggcatcaaataatagacagttatcaattgttttttattgctgacagaaaaccttttgctacaataataaacaacatttacacaatcattctctaagcatttgaaagcacagttacatatagatttgacaaacacaatcattctctagctatagtttcaacaatcttttggtcatctgtgatagataagaatttagattatggaagggacaccaatgtaagtattttatctttcggtcctctagtaattgttgaacggaatagttaaccttaaaacatctaagtgccaaaacatccacacatatattaacccctaagatcatttccaatggaggaagcttttctacatatgttatgacatctaataggtacttctataatagaaaaataaaaattgtgttaatgagaagagtgactttgttaagctctctttattttaatgttaaaatattgaaatataatgatattaaactatttttatatgatataaaattgtctataagtctgtacaagaaatttgtgtgacgtgcaaagttagagatattgaatgtttataaagttatatatgagtgatacgtagttctgaatgccgatttaatgtcaaaataagtagactttttttgaaggagaataagtagcccttatcatttcacctaaaaaaatattcttgtgaatatcttttttaagaacctttattttaaagtaaggtttatatgctcaacaatgtcaccttagctcttatataaacaaggaaaggaaaacaagcactaaccaacacaaacattgtaacaacatcactctcatccaccaaaacacaataaca

SEQ ID NO: 98 - DNA, 4000 bp, Pisum sativum
  source 1..4000: mol_type = genomic DNA
tattatgggacatcttggtgcgaagattgctagtattgcatttagttagaaaatctttattattgtggaacgataagaagatgcactttgttaattcaaactagtttggaaaaaagatttggatttagaatatttggaagattggatttacaatggagaaaagatttaattggatatgctttgaatgaggacttggttctctattctaagttgtttggtagtattcctctggttttctttcttttcacgtctttgccaaattatggcaaaaggggggagaaatggtgtgagctcatgattgatgctagactaactcatgtatgatgttgatgcagttctgatgcggtttatggagtgttattttttggagttttgatgcagttctgatgaggagcattatttaacatatgtgtgcgcacatgctgagacatctggccctaaaatccgtgtgactgtgtgtgctgatttctttatgtgctagtagtagcttctgagtcatgtctactgaccattaaatgtgtgtgcatgcatgactaactgatcttgaatgattgaacgactgactattatagagcgttaatgtgtagcaactgctttgctactaatatgtgttactactgctctgacttctgctactgatgtatgctctacattgatgttcttctgctgctggtgatgtttcttatgagtctgggattcccttgatactcagttgtgtttctgcttgaatagttattttgccaaaaaaaattaaagggggagattgttgttcctttaggattgactgcattatgcaaaataacatggagtatgttcattgtgtttctggtgaagaagggacacacgtggaacatgatgttccaacatgtgtgtgttagcctagaattttcacttagtagaaaagtcaattgaatgttgcattgggaaaatgttaagcattagcattgattgagatacgtcgacagtgatggtatgtcgactagagtgttttgactggcatcttgagatgtggtttggaagaatcttaagagaagatcatgagatatggtttgaaggagtttcatgaataaatatgggtaatcactcgacgccatgtcgaggtcgataccttcgacgggttccaaagacttagaatattgagaaaaccatagaaagaggaaggtacttagcgctatcatcaactacattcgacgagcagtaagatactagtcgctaagagtagttagcgaacattgggtaatggctttcaaattttttcaagtattgacgacgtggcaatgcatcagaagaatatgttgtatgtcttagaagacatgtcgattatttaggagtgaactgtagggaactgttatttttagtttagtatatatagttggcttatattttgtaataaaggtcacaaattttagatactttatatatagaactgaagtacccaagccaaagtgtgaaacattttgtgagtgatatgtgagtctgcatccctttcattgtattttctttatgttttcttaattgaaacatcttttcttttcttcatttactgcatttatttaatgtttttattgtaaatttcttttagtttaactgaagctttattgtctttaaagtttatttttatcttgtttgcatacagacactcataatcacttcatatacatattttcttgtaaacgtgtttgccaataattacttcagagatttttcaatttaatctcacaaactttcaaactcgtgattgttctttaaaaacacttgcgaatagtgtgatacaacatctggtctctgacaatagacatgtgttatggaagttctagttcatatttaatccaagatttactctacaaagaagatttatataaagagatttaatttggatgttcaggtgaaacatatggaacacgacgttccagcatgcgtgtagtgcaacatttggccttggtcagcaagccatgatgaatgttgttaggtgcaaaattctgattgagattcaattagatcttttgcagattgatttgtaatgtttgtttgatttgtttgaaatctattgaagatcaaattagatttaaaggagatttgaatttgaatttgaaatataacaaatcatatattttgttggagcgatttatggagaaaatcaaatccaattgattcaactattattctggatgaaattaaattagaaatccaaaagagaaaagaccataatggattgctatttagggagactcaaatctcatgtttgagaacaagttttgtgccctaggagtaagttctttttgctagtgtttttgattattttgttatttgtattccacaataatgttattagtcatatcataaggattagtgttagatcttggttgtgtgcaccacactattggtttagtcacttgacatagttgttgatttgtcttagttgaattttaaattcatcattcaatagagttattattgaagttgatcacgtgggattaatgattaagagaaagtgagagggattctcatatttaaggggagtcataaatagaaagtcactatgattaggaagaggctttgaacaacacacagttgatgttcctataagactaattgtactaattcaagatagggaatttcctttctttggtatagtgcccttttgatgtaggtgttgtttgcaccgaaatgagttaccaattctcttttgttattttattgctttatgcattgtgatattgttatattattgtcatactggtttaacaagttggaccagttgtcccaacatcgtgtccaacatctatcatctcaggaacaaaatttcagcttccaattttcttgtttagagggagaaaataactacttctatgtcatctctttcatttcagaatcatattgttttccttatttggatgagttgagcttcatctaagatattgggcttttcttggcagttattgcaggatcgatacctttcgagagtaaacttgtttagaagagaggtgttatcagattctagtaggctttcttgtcctttttgtgggttttcgtctgagactgcttctcacctttttattacttgtgagacagtcttgtcagtgtcgtatagagtttttcaggtagttggggtgccaagtagctattcaccacgatcttaggttgcgctttgagtactttcatttttaggaagtagggttaagtatatgagcgattatcttatggtttggcatcatgatgtaatctggtgtatttggagagtcggaaatgatgttatatttaatggagtcgcaaaaagagagaaagaagtggaggaaaatagtttttttttggcatggaattattttttgggtcggacgggggataactcttgcaccttcacatattggctttaaaatcctatcctctccctacacctatagaggatcgagtgtttacactaattctgagtgtggtggtggatcttttttttcccatcttttagtgtgatttgatttgttgatggtgagtctagttgatttggagatctaacctaagagtagcgacgtgattctgaagaaattgtggttttgattctggatgtccctcggttcaaatcttctagattttttctattttttgttgttttttattttgttttcaaacacttcttatggtttgtattttctttttagatttttgtatcttgagattttgtcataactaatatatttattttgccattaaaaaatagtgataaaaatactctaaatacttgtatttttgaaaccaataaaaaaatattcaaaagagtaattaccacgaagaattttatcaaaattcaaatatgcttcttcaaacaaacataacatatatctaaaaaaatttaattaccattactaagactttataaacaatttattttaaagtcaacttttacattctcaacaatctcacctaatttataatttcattaactataaaaaccataccaaaccaaacaaaattaaaaacaaatcccatcaaaaacataaaccaccaa

SEQ ID NO: 99 - DNA, 3000 bp, Pisum sativum
  source 1..3000: mol_type = genomic DNA
gatgtataaaattttttgtacatcagagaacacaccaaaaatctgaacctctttcaagtcatatatttcaatccattcatctattggtttttaatgttttcataacattttgacataaacctttttgttcataaatttcaatcattaagaaaaatattctaacctctttagcatttattgtttgtatatttcttgaattgaatcgttgaaatagaatatcaaaagtatcaagatattcaaagatcattctaacttgtgtgtaaattatcaattgtctaaatctgtactttttcattgagtgtggtaatattctttgtttgtaagaacgtggtttgtactttctataggtgtgctcaagtaggaagttttgttctttatttttgttggaaagtatgatatatttttctaacaacctttcatgaacgttgaccgattaagggaaatcacgctaccaatctttttacttaatcggttttctaacactttcgtcgtgtgaaaaagatgtttgtcatcatcttataacatgctttgatgaagacaaaatgtattatcttataagaaatattaaaagagaaaaagtttgaaataaagttttcaagattgtttaagctttaataatcaatggattaaagatgaaggtttaaatacaatggtaaatcatatgtatataagtattaggtgctcagaactatcttcatctaaaatctctagcatgtgcatatactttcacacaagtttaaaagttttcaaactttcatttttgtaaattgttgaggttgaattcaactataacaaagtgtaaccgaattcagatggcaaaaagagatgaaattaagcaatttgtattcaactataaagatatttatttgaatacaagttctgttgtatttagatactaagatgtttgtttgaattcaagacttaaaagttcgaattttcaaaatgtagaaaggactatattagactacacacatcctgtagccgaataaacattcaaatcagtgcattttttcactactgaacctatttcagagtagttcaaagtttcatgattgtttgttaatgattgtatctttataaataggtgttttaggatgtataaaagtttttgtacatcagagaacataccaagaatctgaaccactttcaaatcatatttttcaatcaatttatctactatttttcaatgtgttggcccaaacacaaaattaattaggatttagggtttgttatatttgtcacttaagggttaattcacttaattgtatataaagaaatactttgtaattcagttttgtaacacagtcctaatatttaataataataatcgttatttcttcgttctctcttttcctctcctcactaaaagtacctcacgcacaagcaactcatccccataaaattgacttatagggtaaggggtgtcactatatataaatcatttgtggccactatctttaaccaatgtaggacttgggttttttcgaatacacccccttacatccaacactatcggtcttggtgcgtggatataaatggtgggtgacccgaattatcgatatgcaagactttgtttttccaatacacccccttacgcctaatattttttgggttgggtgtgcaatatataaagtgtggatgacccgaatcgataaactcgtgataccatgataaaattagagtttgaactaactcatccttacaaaatcgacttagagtgtgaggggtgtcactatatataaatcatttgtggataatatttttaatcaatatagaatttgggttttttcaaatactaaataactatatttaaatttttataatatttcttataaaattgttttataaaatattataataatatcaagatatctaatacgatatctaaaaaaccatgtttaaaaagaatgacataaaaactaaataaaaaatgttttaaagagactctaaatgcttgtattttttaaacaaataaaaatattcaagagcgtagttttagtgaaacttatttttagaaacaagggtaatgctaacttggctctaggggcacaagataagacactcacttatagaaagtttgtattgaaaaaatcaattataaaattaattttatatctttattaaaatcaatacacaattttcaacacaaaactaatttctttagttcttatcttgtgctcctagggcacaagttagcattatcctagaaacaataaaaagaaaaatatctctaaataattactacctccttcttttatttttcctattatacctttaaatatttattattctcactccattcaattatctcaatttgttttttcaataccattaatgaagcataattttgtaaaattcttcataatttatttttgtcataccataattattacatttcttaatacgtgtgaaaagtcaaaaacgacttataataaaaaacggagggagtatattttaatttctataataaatctttttatattttgttgcaaaatattatattaacagaaatatatctgataccatccggaaaaaattctatttaaatagaatgacataaaaactaaacaaaaaaaatatgttttaaacataatctaaatggctgtagttccaaaaccaataaagatattcagaagattaatttttgttataaagtgtttaattaaagttcaaatcaacttttgtatttttaattttataattctaaatggaaaaacattacactcctaatacctaattaaaattgattttacattttttatgaaacctaattttaaaaagaggaaaagaaaaatacaactgtaaataattattcttattcaaacataacatatacccaaaaaaaaaagttaattaccataaataaaactctaaaaatacttattttaaagttaactttcacattctcaacaatctcacctaaaatcataattccattcatttataaaatccaaaccaaaccaagtcaaaacaaaaacaaatcacgtcaaaaacatcaaa

SEQ ID NO: 100 - DNA, 312 bp, Vicia faba
  source 1..312: mol_type = genomic DNA
tatatatgatacattatcaaaaaattaattttaaatagaaataaccaaaaaaactaaacaaaaagttttttaaataactctaaatgcttgtatttcctaaaccgataaaaatattcaaaagagtaattaccatgaaaaagaaattaattaaaaactttaaaaacatattctttaaaaactttaattaccataaacaaaactctaaaaagtttattagaaagtcaactttaatattctcaacaatctcacctaaattcataatttcattacctataaaaaccaatcctaaccaaaaaatacataaaccacata

SEQ ID NO: 101 - DNA, 3216 bp, Trifolium pratense
  source 1..3216: mol_type = genomic DNA
ttgtgtgtttgccatggaagttttcttacccctgctgaatttgtcaagcatgctggtggaggtgatgtggccaatccattgaagcatattgttgttagttcaagcttaaattaatttgaggctaaaagagaaggaaactatgatatgagtaatcatatttttatttattattattggttcatttgttcagtcttagactttgaaaccatggatgtttgaggatctgaatttggtgtttcttattgaggtatatttttaccttggtcttatgtatgttgtgactttgggacctctatgaggtgaaatggtacttgctagagtttaaattttatgttaattaatgttaatttaattgtatttttagactatttagtatctctctgtttttgtggttaattttagttttttttaaatgttttaatatataattaaattttattatcacttctattttgaattcatttttattgaaaaaattattcttttattatttaacaatttatcatgttaaaagctgattttactgaaaataaattattacaattttcctctttatttcttttgcattttcgttttctttttttgataaatattatcttgtattttctaaaatttaaaattggctaaatagggttttaccccccgcaaaataggtcagttttgtttttccacatgaattttttttttggattctcccctgcaatatgaagattccatcgtttacccccggatggccaaattggattgaccgtgtcaatctgcttatgtagcatgctgagtcaatttttttaattctttttcatttttgttttttatttccaacatggatttccttttatgctcccatgttatgtcataaaaaaaaaaggatttcatgtccagttggccatccaggagggtaaacaatgaaatcttcatattgcaagggagaaatcctaaaaaaataaatttgtaggaggtaaaacaaaactgacctattttgcaggtctatttaccctttaaaattttattttttgtgatattctttagactttattcaactatgtcctcttagatttttcattctaaattcgtttttattaaataaatcaagtattatctcataaaaactattcacaattattaaattctcatttcctattatcacttgagagttatatattttgtttatattctccttaatttgacatcaaagtttctttgaatagaaaattcgagatttcatttgaatagatatatggctgagttgtgtatcatcacactcatatttagagtaaaccactctacttattgctccttaaactcctacccgaacctacttttatgtttagactcgacaagtagagaaattgaaaacacatatgggcttcaggaataggcctcccactcaggaaaaaaaaatcacaaatttaaatattaccaatttggtttttatttgattcttattgaattttttatagagaatatccaatgggtgacttaatataatgttattcttaagtagattcacaaattgtggccaaaggtgttatgttgtcctttcttcatatcttagtttttggttttcattcttaagtagattcacattttttacttacccttcatcttataaaaagaaatattttagaattgatggacctttattgttcctttgcaatttagtagttcagctcccaccaattttattttaaatttacactactgtagcatgtttatgtttattaacatccactaaattggaatagctagcagcgtgacttttttataaattaaaaaattaaaaatagagaaattattgacatagacttaaaaagaaaacattaacagaattgagttttgtgctaagatttgaagttttctccacaatagacttaaaaagaaaatagagaaattctgttttaaaatgcaggtaccatttcccccacaatgaccgtaatcgagttagatttctacaaaatcaaaccccactaaatttggagttacagagagagattaattaccgttttagtgaaggtatgtcttccaaaattctgcacaaaatctgccttcgagtaacaacttcaaagcttcgcacacaactaaatttggatcaataattcaccaaacggataccgaaatgaccgtaatcgagttagctttacacagaatcaaaccacactaaatttggagttacaaagagagattaattaccgttttagtgaaggtctatccaattactaattttgcagaaatctacttctgaccttcaaattcaacatcgtataccaaacacatcgtaactccaaattcgacgaaattgaaatgataacaagggtaattttgttagctttccagacatgtaaatactattaaaaaatgagctacagggtgagacatgacaaaaatatcagtagtaattccatacaataattaatttggacagaccttcactaaaatgataattaatctctctgtgtaactccaaatttagtggagtttgattctgtggaaaattaactcgattgcggtcatttcggtaccattttggtgaattatagatccaaattgaaatctgtgtgaaactttgaagttgagactcgaaagtagagggtaaaaatccacattttaaaatatgggggtaaaattctgatttaatcaaaataggggggtcaaaattgtatttaagccaattatctatttttacataaggcatctagaaattttgacgtgctttcataaatcctcattgtagtgactcgagtcactgctaaatatttatttaactttattattattattatcatagtattttagtcaaaaaaaatattaatatactaaaacaaactcattaaacaattgttcaaaaaattaaaaccgattgacaatgcagttattcttatatttctcaaaagagtaatgattattcattttatttttaaggaaaagagtaatgattattaggaagactctaattgaaattgtaatattatcttatacttattcaaacataagttacacaacggatatatactttacattaatatcaccataaaaacttaaaaaattattccaaaaaataaagtgttttataaaatattttgtttaaagtcaactttttaattctcaactatctcacctaatctcatactaatccatctataaaaacataataggaaaaacaaacaccacaccacacaacacaaacattgtaacaatctcttatcaaccaaaaccaca

SEQ ID NO: 102 - DNA, 2997 bp, Trifolium repens
  source 1..2997: mol_type = genomic DNA
acatctaaaaacatcattccattataacgtcctctatttaagtagcggacgacatattccaaaaacttctcatttttataacatacgctacttaagtagcggaagggtaaaataaaaaacgcgtagggttcgtgagtaatcgatatggtgtcaaaagtaaatttcattgcttagtgtaatgagcttatgcattatccaacaacaaagcctctttaaaaatgagttaaatagcccaatagcaaggtccaagtccaaggtccaagtcaaaagacattaagtcattaactatattttcccccatcttgcagaccagggacaggaaaatggaccaattagcgcgagtgcgcgcacacacacacaaagagaatcaagaaaggtggaaaaagctttaacctaatacctatctaggacctagaggaatttcttaaaaaataaaacttttacaagaagagactagtcgaaaaggataacaccattaagagactacaaatgccccataacatgatactaaggccaagccctttttaaaacaaggaataactctaaaggcggtgaacaatgagaataacccaacaattaaaatgcacagtgagttgcgacgtacctgaagcacctaaaagttttatactaaacttgacttgtaaattgacaagtaacaaattagggctttgttatttggttataaattctcattttgccaccctactcattctctcgtgcgccctatgatgttatcaaaatacatctttccgctactttggtagcagatacactcaaaacaccctaccattataacattctctacttaaatagcggacgatattttccaaaaacttcatttttataacgtccactacttaaatagcagaacggtaaaataggaaacgcgtaaggcgcgcgagtaaccgatagggtgacaaaagtaaatttcattgattagtgtaatgagcctatgcattatccaacaacaaagcctatttgaaaatgagacaatttctcttcccaccctccacatttctttttcatccctatagcatgcaaaaagaaacttggggagtgggaagagaaaatttctgaaaatgagttaaatagcccaatagcaagatccaagtcaaaagacattaactattattctaaaaggtgattgctcatcccacccccctaactctttttcacccccctaaaacccaaaattacccctgaaaattcaaaaaaattgaacgaaaaagtacggattttgcgatccgtgcttttgaaaagtatgtattacaaaagtagtgcggaaaaaatacggatgacgtagtccgtacttttccttaataaacccagaacatcagtctttcttcttcactttctcttcttcacaaacacacaatcaaaatctccatacgtgatccttgcgcgattccgtccattggaggcttaaaatcaacattggagtgttgcctaactgcatctacacgcattggagctcaaaatcaaggtaaatttcgtaacccaccgtttagtttgcgcttacctactttctagcttactaggtttctgacacactttagcacggattaggagatccgtactttagcacggattatgcgtgccgtacttttctgggtttggaatttgctgcgtagctcactgacttgcttatataatctgcaatattgttacattaacttagaaaacaagtaacctaaggactatttggtgcattgtagtagtatttaaacgccaatttcatgcttagattatttttatgccaatgtataaaccataattagtaactaatgtaattcaaaaatatgcataaacttatgttatatctctaaccaaatcaaaattcaaaatttcaccaaaacctaattaaatataacattcaacataatagacattcatcctaacaaccaataatcaacaatgtaaacaaaatcatttacagcatacacattcaaccctaacaaccatccattcatctctaaacaattgaaaaatcaaaaaataaacctatacctaaacctaatcaaaataataaatcaaactaacatgcattcatcatagtaaacaagaatcaacatcttcaacaacataatttcaaattaatcataaatcagagatttttcatacatgcaatcccaaaaacgcagaaaattgagatgaagaaagaggaacacaataaagtgatcaaaattagaaattaacttacttgatggagtaggaattgataatgtatatggattccggcctccaaaacgaatggttgatgctaattgataaacttggatgcttagggttcttatttgttcaaaacccagaaacgttgaaatgagaagaggagttaacgctctgtttatattaaggaaaagtacggatctcgcgtcccgtactttttccgcaacggatcttgtagtccgtacttttaaaatagtacgtatcgcgtttcccgtaccaattttcaacgtagggggtggaaaattcatgtaaaaatacggctcgcagggaaactcgggggtggaaaagaaaaccggggggtgggaagagcaaacttcattctaaaactcattaagcagttgttcaaaaaaataaaaccgattgagcatacagttaaatgcttatcctatttttcaaattcttaaaaacataaacattcaagagtaatgattattattttttttcaaggaaaagagtaatgattattaggaagactctaattgaaattctaatattttactgtacctcttcaaacataagttacataacgggcatatacttagaaccaaaaaaaataaataacggatatatactttacattaatatcacattaagttttatcaaaaaaattagtcaagtaaaagttttcaaaataaaaaaattgattaaagtcaacttttatattctcaactctctcacctaatctcatactaatccatctataaaatcataggaaaaacaaacaccacacaacataaacattgtaacaatctcttatcaaccaaaaccaca

SEQ ID NO: 103 - DNA, 2997 bp, Trifolium repens
  source 1..2997: mol_type = genomic DNA
aaggcccaacttctcaaagttgctgctgctgaaaccatttatatgctttggaagtataggaatgatatttgttttgacaaccaagtacataacacaaagatagaggaaaatattatcaatacaatagtttatagagggtgatgttatcctaagcttagaaaacatgttgctcttatgctaatctagcttagtttctctttttggtttgtttgtcctctttgatggttggatctttagatcaccttgtacttacttacttttttgaatggaataaatctcttaattcaaaaaaaaaataaaaaaatagatggctgatttgtgtatcatcatcaactcatattatagtaaaccattccacttattgctccttgaacccctatccaaatctaatctttgtatcacaaaatggcccataactcaacttgtactactagaacaaacacaattgttaacatgcatgtttgcatagctcacaatgagattctccacttcatatctaatcagatttgttgcagtgtgtcaatatgatttataaaggaaagagtgataaataatttgattgtagtaattataaaatttagttctaaattaagtcaaaatattatcaagagatacaaatacacattatgaaaaaatttgattgtaatggaagagtctaatgtgcgacttaatagtataatgctatccttaagtagattcacaaaaatattttgtgctcccttcttcatatcttagtttttggttttcattcataagtagattcacaattttttacttgtctagaatcattgtgcgatttttacccttcatcttttagcaatgtgcttattaaaaaagaaatattatagaattgatggacctttatttttcctttgcaatatagtagttcagctcataccaattttcttttaaatttacactaatgtagcattatttattaacatgcactaaactggaatagctagcagagtaacttttttttataaatttaaaagtttaaaaaaataaaagaaattataattgacatagacttaaaaagaaaacatatgctacaaccttgttattatatttttattttaaagaataatcacgcgactaaaataaatttggttgcacgttaataactatgctaccaatttgaaagaaaaaaaaaatagaagactatcaatgtttggctcctttgtctttttcttggaaaatgactcacaagggatatctttagggttttgattttcaagtgcatcttttttttttttgttaatttcaaaacgaacacttaatgaattagactttagcatatcaaatgattgagagctttaattccttgtctttctaccatatttttcctgttgttttcatgccttgtcctcgttattatttccaaaattccaccctaaagtatattcacgttaactatgcatggacgtactagtagtaccccacacgtttataacttatgtcgctttcataatatattctcttcagcgacgtaagattcttgcatcctatatattcttttttaggcgcatccttttctgacttcaacaagcgcttattgagttagaatttatcggatccaatgattgagagcttcaattccttgcttttctatcacgtttttccttttgtttccatgtttgttctcgttattatttccaaaattctttcttagcgcatcttcacgttgaccatacatggatgtactatacctcacgcgtttatatcctttgtcgcattcacaatacattctcttgagcaattaatggtttaaaattcagctaagaaaaattcttgcctcttgtatattcttttcctaaatgccccacttctaaataaggtatattctcccccaaaatagaaaaattggattcattgccattatgaccaaaactaatgttactttgaactacctatcttcattcctcatcgatgaataaattttgaaccattatccacaactgtgacgacattccattctagagtttcaccatactttcccccctcttgcagatcacgcacacacgcacaaaagagtcaagaaaacttaaaaaagctctaacctaatacctatgaatgacccagaggactttcataaaaaaaagcttttacaagaggagatgagtcgaaaaggataacaacatttttataacgtccgctatttaagtagtagaagggtaaaatgagaaacgcatagggcgcgcgaataatcgatagggtggcaaaagtaaatttcattggttagtgtaattagcctgtgcattatcgaacactaaagcctctttcaaaatgagttaaatagcccaatagcaaggtccaagtccaaggtcctagacaaaagacattaactattatactaaaactcattaagcagttgttcaaaaaaataaaaccgattgagcatacagttaaatgcttatcctatttttcttttttttggtacaaaatcctatttttcaaattattaaaaacattcaagagtaatgattattattattattatttttttaggaaaagagtaatgattattaggaagactctaattgaaattctattattattattattattattattatacgggtcatgctaactagtgcccgaggcactagttaaggatactaaaaagagcaagttttgcattgataatagtattctttatactttaaaaaagtaaaatacacaagttccaaaacattttttactatttgtagttccttaactagtgccccggagtaatagttagcattttccttattatactgtacttcttcaaacataagttacataacggatatatactttacattaatatcacaataagttttttcaaaaaaattagtcaaaaaaacttttccaagaaaatatattgattaaagtcaacttttatattctcaactatctcacctaatctcatactaatccatctataaaaacataggaaaaacaaacaccacacaacataaacattgtaacaatctcttatcaaccaaaaccaca

SEQ ID NO: 104 - DNA, 3256 bp, Trifolium subterraneum
  source 1..3256: mol_type = genomic DNA
atacttttccctattgtagatcaaagatggcaaaatggaccaacttatcgcatcgttaactcatatttcttttaaacttacattaatatggcatgatttagtaatctgcactaattttttgacatatctatcaatatggctttattttcattaaaagaaaaaaaaacacacaaaataaagaaattactgacatggacttaaaaaactatgatacaagcttatttttaggttttattttttaattttaaggaatagtcatgctaaataaaacaattaaaagtttggttgtacgttaataatgattctacctaagcgttaatttgaaagaaaacatttagtgggagactgtcaatagtttgctcctctgtctttccttgtgaaatgactcgcaagggatacctttatgggctgatttttaggcgcatccttttttgacttcaacgaacgcttattgagtcagactttatcagatccaatgattgagagttttaattccttgcttttctaccatgtttttccttttgtttccaagtttgtcctcgttattatttccaaaattcttttttagcacatcttcaaattgactatacatggatgtaccggtaccacatgcatttatatcctttgtcgcattcataatatattctcttgagcaactattggttcaaaatttagctaagtcagattcttgtctcctatatattctttttctaaacgctccacttccaaataaaggtatgttcttccccaaaatagaaaaattggattcatttccaatatttccaccactaatgttactttgacctacctgaccttcattcctcattgacgaataattttggaaccattgtccacaactctgacgacattccattcttgtgggagaatggaccaattcgcgcgtgcacgtagacatacaaacacacacacacacacacaaaagagagtcaagaaagacaaaaaaagctctaacataacacttatccaggacatgggggaatttcttaaaaatccatttacaagaagagaatagttggaaaggataacaccgttatgagacttcaaataccccataacacgacatgtcaaactatggccaaaccatgtttaaaaacgaggaataactctaaaggatgtgaacaatgagcatgacccaaaaaagtgcaatgcctagtgagttgtgacgtacctgaagcacctaaaattttatactaaacttgaatagtaaatggacaagtaacaaacaaatggcgcactgattcctgcgcaggagcacaaaaaacacatgttgaacccaaattgtcctgaataactgataagtgctaaaaaaacagaaaatttatgtatttatttcaagaacctattaacttgttttacacaatattggtttaaattagtgaatcagaaattttatagcatttatacctatatctaactatttttgtaggaacatggaagcttaatggaattggactcaaagaacttaatattttgatcaagtaacagtcgttgagctagcagtagcaagctgagtcagagagtccagggaaatccaacataaggttggtcgctaggcgagcaagcaaggagcgacgaaagtcagagagttacatttttggatactagggttagtcgctaagtgactagtagcgactagcgaccatggagcgatgaaagtcagaaagttccaattttggatattaaggttcgttgctaggccaacgaccattggcagttcagagtaaatatttgttgatgcggcttttccttccattgaagaaagcttggtcgcctagaaaaatatgtattttgagtttttgtgcacaaaaggttaaaataaaaatatgagaaacttttcgctaaatacgtgtacgcgagactattagtcgcagatgcaatttagcgtgttttcaaaatggaaatttcgctaatcgcgcgtatgtgatgctacacttagtgcacgcaaaatccacataaatcatgtatttgatagaaatttaatgaaaaattaagggtttttagactctatctttgatggctacaggattgttgaagagttagggctttgtgagctttcaaacaccaaccatcttagattcattctatctattactctcattggtccttaatataagagaaattttatattttagattcattgagaatctaatgtatttagtctttattattcactagatacattagattcttaatgaatctaaaaaataaaacttctcttatattaagaaccggatggagtaattcattacttcaaatcttttaaattagtattcatttttaacctttgttaattggttagtgtaatgggcataaatgacttattccagttatcccatgcattatccaacaacgaagcctacttgaaaatgagttaaatagctcgaaaaattcccttccccccagtccatatattttttctcgctctgaattttcatttttgcccttgaataaaacttcggaacgcgttttttgaatttttttccttaaatacaaacttcagaacgtattttccgaactttttccaaaactttgaagtaggaaaacttcggaaaacacgttatgaagtttttattcaagagcaaaattggaattttgaggaggaggaaagacatgtaagaggggagaagagaatttttctcaaatagctcaatagcaaagtccaaggtcgacataattatatcctcaacatcatgttgatcagatatcgacccgagtcaccactaaaaaattatttaaatttaaattttattatttttattattattttagtcaaaactaaacacattaactgttatactaaaactcattaagcagttgttcaaacaaataaaaccgattttttttttggtaaaaacaaggctaaagaaaacattcaaataaaaccgattgagcatgcagttaaatgcttcatatttttcaaattaaaaacattcaaaagtgaaatgattactatttttttttaaggaaaagagtaatgattattaggaagactctaattgaaattctaatatcacacttctacaaacataagttacataacggatatatcctttacattaacatcacaataagttttttcaaaacaattagtccaaaaaatattttgattaaagtcaacttttataatctcaactatctcacctaatataatactaatcccatctataaaaacataggaaaaataaacaccacacaacacaaacattgtaacaatctcttatcaaccaaaaccaca

SEQ ID NO: 105 - DNA, 3017 bp, Lotus japonicus
  source 1..3017: mol_type = genomic DNA
tttttgaacatacatagcggtaattttgaagccataaaatttatgttttgaaacatacatgtataacaacgattttcagtcgaaaactttgttaaatttgttttttttttacacttcgtatttagtgatacctgaaattgtcaataaacaatatattaattgaatgtcgtggatttccatagtatacatataagcaccgtaaattaataaattccaacttacccatggactcaatgtctagtcatatatatctttagttcaatatttgttcagttttgctgacccctttatacctttttttagggttgtccatatcggacagtttcgggccaagatcgtcagttttgccggttgaaaaacatcaaaccatcagacccgccatcacttgctcactaaccacatgttgtttaggttcatattttttgggttgtgattttgatggtgagttcacgcaggtcgtcatatcaagacactaatcaagaaaagaaaagaaagtagatgaaagaaacaaacccatacaagttggatcaacattcaacaggttcatcaacattcaacatatgaaacaacacaacacagaaaaggtgaagagaaatcaacactcaacaaataagaacctaaacaataaatacacacaaatcaacatttttcatcttcaccatcatcttcttacaacgttccattgtaagtgcttgagtggggatagaaaagagaacgaggtgaaggaacgtagaacctagaaccgcctctacaaggacgagatatcatccgccgtgatgaaatcactcttccaatctgcaacagtgggacgaggagatgaagcttccacaaatttgcgactgggagagagaaagggtgaggatgaggttgatggtgaagatggcgggcgacaatgatagtgaagaaagtgaaggaattaaagagaaggaaaaataatggatctttgtgaggaggttcttgtggcaatgggagaagagacatggtggtggtgagtagggttttccgattttctcaagcgggaaggtgaggggctgagagtgatagagaaattgagagagtgtggtggctagggttggaattagaggagtctcgcatttttttattataaataatacctaatttggtcgggctggagcctagcctgaaattgatcactgttaattacaagaaatcagctatttgaaccatgcgacctgtgaactaaactgacccgctcggtttcacaattttgttgttgttgttgtgtttttacaggtttcgaccataccagatgtttatggacaacccaatcttttaaaaaaaataagaagcattataagaagcattgttgatgcttctgtacacattatttcaatcatagagtcatcattgcatgttttagtcaatcgcatgagcaattagcaaatgacctcactagatgtaaaactttttacatgattaatgcttaattgcccatttttttcattctttagtagccccttcaaccatcataagaaaaagaccctgtagacagcaaaattacatttggaacaccaacaatgatatatacacacctcattttttaaacacttaatttccacctatttttgtttctacctatctcctcttatcatctatcacatctcatactttttcttttcttcctatctctcacctcattccacctcttccacttctttttgaggtgtgtaagtaacattttcctttggaacacacaattggatgaatcaattttctccatatattatataatatcactcatgtgatgtatcacttaaaaggaatcctacattcttgcaagaatcactgggtaaacatgcataaacatgaaaacagaataaaatcgaaagctagcatataataacatgtcaacttatgtacatgtacaacggtaatattcccaacaaattatgacatgtgttaattttaataattttctcgaattagaggaggattagttgtgcaacccgttagaggaaaataaacacaatgattctaaacatatttgccattagaaaaaggtattgaaatgtgatgcaatcaaaagagtattttgtccctacttataacttttcagcgccttggaaatggaaacatgttcataaatatctactccaaatctaatgaaagaaatagtactacatactcgctgtcctcacgcaatataaattggttgtgatggatcaactgtatggctatcaaaaaatctacatgtattaagaatcataacattataaaaaatagcagcatgaacagaacggcatgcaactaattaaaattcgaacattatatgtttttctcttttgtgtccggttgtgattcgtttataaaacttgtctcaagagccaattccattaagatatgagaaaaagtttgatgcaccgacggtgtaaaaaattttttttacaccgtcaaccaatcagatttcaaggatgtgagaaaatctctcttttcatttaatttctttaattgacatgtcacatccttgaaatctgattggttgacggtgtaaaaaaactttacaccgtcggtgcatcaaaattaaactcttaagatatagttattttcaactcgaacatattatttatatctgtaatctttaatatttggaaatacatgtttgtcagacataaaaaaaagtacatttagtggaggtgttgaaatcgccaaatgaaacacgaaattatgtgacggactttctcacgtattctagaaaaatgtttaaaaatatagaagagtttagttcaaaataaacatgcaattttgtgagagtgcataaaccgtaaaaaaaatgttctttttttctttagatactttgacgaaatgtagccataaaaaactgtcacaatcaccaacaagatccaacacaatttataaatttctgaactctttaatcatttagttctcatagtttaatcacctaatataatttttgagtctcgagtaaattaatcttgtatacaaatcattattgtttgacaaatagaatggctgtgtactaatatttgaactatata

SEQ ID NO: 106 - DNA, 3000 bp, Lupinus angustifolius
  source 1..3000: mol_type = genomic DNA
ccacaaaagtctatctatacacatgaatagtaaggggaaggaataaaactcataatattacaacttgaatattctattgcccacctatttgcatagtagaaaatatgttttaggttaaaaatctcacctttaaaattcacattatttttcattttccaaaatgataaaaaaagtgaatctcattttattagtacttatgagaaaatacaaatgatgagtaactaatacattaaatataatttcacacattaattataagtgatatttgatttatcaaaactttatgttttacttatagtccaaaatgtgtatcattatatacatcattttattctcattgattaaaatatataaagataatatatatttaattatatcttgtttttctaactaataaaaatgagtttgatgtgaagtgatatagcaaatgagtctcttttatttgtgagcatgtcttttatccactatatgtattatgtatctttcatctttgttagtttttatataactaacgtatcatatctttttatagataaggaacaaaccatttttaagatgggaatggagtatctccttacataaggagataatttatctcctgccagtgaaatcatgacatgtgtcatgtttcaccattttctataaaaatatttttaaaaataaacaaaatagtacacatgtcacatttttattggttagaggagataagttatctccttatataaggagatacattattctcttttaagatatagtcattcctcttaacaaaacgttttgtaaacaactttttttgtttggttagaaaatatataaagttactatatattttatttatattttattttttctcaaataaaaatgagtattatcacactcctactcattttactcgaatatattaccttaattgtcaccatgtgttaggccacatgcattggaaaattcgttgttttaaaatttgtaaacggaaatgaccaaaaaggacgtgtcactcaaagcattgtgatgcattatgcaaagttgcacgtacttgtttgtttcttttcgaacggtcatgtccaacgtttccaacgcgcgcaagtagcgaatatcaatgtaattattgttttttggtgcacggaaaaaggaataaagaacactacaatatcaacggcaacatttctatataatatattataataatatgtaacatacaaaattcaattaatttcatattaattatttatccaacggtcatcaagataatacattttgtaacacaaaatgagttattctaatataatttttgtaaaggaccatgtcaagttgtttgtaccaataattctaaaatctcaacctcttaaaattgatccttctattcaagatcttttataatataatttttatctctcgattactatctatcttttatttacacaagcgtcttcaccaagatctataaaaatattatttttggtccaaatttcatgtatttcataatgtatctcttggtcaaagaaaaacatttatagcttggaagactttttttgtgcgtttcgtgttgctttattatccatatatttccttacttatggtcaaccaactcatgccaacccaaaaaatatttaggatatattctcaactatacttttttggtacattatttatttccttacattgatgcctattttagaccatcttttttattgtattctatttgagtttattattattataatttattttattcatccgacgaggatataaactaaaaaaatagagaatatttgttaatattccacaattactattgtgcaatttatatctcataacccaattttttattaaaataataattattgaatttaattatttgcatcatttcaacccaaccttatttgaaatgtcacaatgctgtacacttttttttctctcaccctccgatttataattcttctagtttattattttaactttaggatgagataatattttgaacatgtttttctaatcttcggatggagtaaattgtatctctaaactccaaaactatttgatccctactggtagaaatatggaataatgagttttgccttttacaaacaattaattttttcatttagaaaatagatttcaattcacgtacagtaacaaatatcatttcaatgagttattgactctccttttactacaaaaatattatttattataaataaaaagataaaagtgaatatactattatttttttttttttaccttacttgtcttttctattaatggatgaccatacttatttatggacaatattacatattttgttgcataatttgtaccatttcatatggtataagttatgattggtaaaaaaaatacttaataattaaattttaacttaaatatattttttaaccaatcacaatctcatgaataaaatgatatatttaaattagaatttaatttttaagagtgtcttacctctaacaaatcacataaataaatagaatgataaataatgggtgcacatatgttgtcccatatttagtgttacttcatatgtcataaccttgtgattggttaaaaaaatatatttaagttagaattgaacttttaactaattttctaacaaatcataaaatcatgttacgatactaagtgtgagacaaacctatacaaaatggatggggatctgttgtccccaaccatgttaccaaccacggccaatcacaatgaaacaaaactgataaagttatgattatttaagttttttagttgacgtgatataatatgattgatctgagacagagtttgtgatagagtttgagacaacagatccttatccatacaaatgatacttcttacctcctttataagtatgtccacttactcaaattataccttcaaaagatttaaatatccaccacttgaattttatctttgttgtcatgaatcctaagccccattaaaaggaggacaaccccattgaacattacaatcaacaactctcacccacaaccaaca

SEQ ID NO: 107 - DNA, 2993 bp, Lupinus angustifolius
  source 1..2993: mol_type = genomic DNA
aagattcttcattaattaaattaattatgaatattttatgatgattatataaagtaaaaatacttaaataaattttcttatttatatttgaaattaatttttaaaatattatattttaaagttgttgtttcttaatgcttcttattataagaaatcattttaaaatattatgataataatttttagagtaaattatacaaacactcattgagttttagtaaaattaaacaaatataaatcaccctttatttatactaacgtgtggacatgcactaccgtgcctgtcaacccgctttcatatcgtttgtaatgtatagtttgtattttataaattcaaatataattttttgccctaaatttacggtaaaatttattcaatttgtctaatattcaatttttttaaatacacttttaaaataagaagataataaagataaaataagaaaataatattatatggttttttggtagaaataaaaagatattaagggcaaaataggaaaacgcaaattacaccatgatgggtggttattttgtgttttccctttattaatagtatagatatatacattacattgacgagccccataggtgagataaatgtaatattgaaacaaaaagagtgataaatataatcaagttaaataaaatataatattttgtgtaaacaaatccaaatagtacaagaaccaatattttattattatttttgattctttataatatagtataagagttatttaaacatcatcttcaaattttggtgcagtttgtaacaaactaaaaataataaagatattgattactactacaggagaaataaagagtgacgagtgtaataaagttaaacaaattttgctatattgtagagattaatccaagtattatacgaattatatatttccattattttttgtttggtacagtaaaatgtaagtgtgatttaaacaatatctcctttaaattttgtatactatttggatttctctccacaattttttttgtttgtcacaatataatcgattcgtaaactatttggatttctatccacaatatatcatattttatttttaacttaattatgctcaacacttttttgtttaaaaaatcacacttatttctcattgagaggtagatagtgtaatgtataaacataaagaggggcaatttgtatatgtataaaaaatatgagtgatttatgtaagtacactaaaacccaggggatgccaatatactttattgtaatttacattttgatgcaatatttaatattttcttttatttttaatttgtttatttataatgaataaatttgtaaaagtttcctcaatataaaaaaggagagaaaatatttatgaacaatgttatttcccaccctttcctttagtatagtttatacttatatttagtgttgtgataaacgaatcggaccggaagtctgatcggtttgaccagtaactagacttgaaaatgattctgatttatagtcttaatcgagtgagggatttgaccacgttggaatggagaaattttggtccaaccatatgaaacccgtttgaatcacaataaccctggaagtaaattagatagttcactcaaaaaatttaaaaaatgaacactaatctaatgttttgttttactctcgtccaaaaagacggaaaaaatattatttcaatataactatattcatttattgattatataaaattttgtacttatatataatatatatttatagataaaaatgataaattatatataaaatcatctgattcgactagatggtttaactgcttaaatcgattaaaaataatataaaacagtttcgagtttgatattcgattcaggtttcacaatattgattctattacaagtccaaaatggaatgtcataaagttaaattatgctggtgtaattgattgatcgtacaagattgcattccctctatagtatatctcttagtcaatattgaaaaagggttttattcctttttaggattttatcatgagtttgaaatttgtgtgaggtaacgttttttatactgttagcacatcatctattattgatttatattacttttgttttaaagtaataaatgattaataaatcaataattaatgatttactgacaatgtaaaaaacacttacattatgagtgtatacatattaaatcattttattattacaatattgtcaaccaaacacaatttctcactaataatattggaaatggttattattctttcaaggtaagagtgtctaacatgttatttttgtaacattttccttactatattcaaaatattaagagacattaatctagtggtataagattctactagttctccaggattactaaaatgttaagtgttcgattcttgagagagcaactaaacaccccgaaagagattagttttttgtcaaaaaagtaaaggaatatcattgacaatgctaaaaaacatactacaaatcatgtcccaaaccaataacaataaaagtaaaacataaaaataataataaatctttatttttttaatttcacataacatattttttatttgcttgaaatatatatactatgggagaacaaatccttacttattaaagatgataaaccaatatcatctctaatattgtatatgaacatggtaatatgtaatataaaattatttttaactattcccattttaaataaattttaaaataaaaaattatgaaatttccatttatatcatatttctgtattcagccaggagatattccttaatccttattcatggtccctagaccttcaataaaacaataatatgtattaaaaattaacaaaattaaataatttcaattcaaatcaaatatggatttaaaactttctttgaaatatcatgcatgaagatatgatttagatttatgagacctaaaccagttaaaaaatttctcttatatatagaacactcccattacacattgcaataaacaacacctcactataaaca

SEQ ID NO: 108 - DNA, 3007 bp, Lupinus albus
  source 1..3007: mol_type = genomic DNA
tttttagaaacacttatttatcaccgtatgaacatatatgaatatatatatatatatatatatatatatatatatatatagggttgggatactctccagtactggaatgtgagatccaaattattttgaagattaaaaaatattttaattgaaaataaattgaaatctgcatcgttaaaaaaatttaaacgtcacatgttatcatctctgtctgtactacacctccaatataggataggatccgcattatatatatatacatatatatgctgatgatcattattttaatttttaatatataaaataagtttatattaataaaaatttaattaatttttttcccgaaacatgtgcttatacataaacttcctaaaataatgttgggaatatttaaaatacactattctatgaagtctgatattacctaaaatgaggtggaggtgagaatgcgatcttatatattaagctcaagttccttagtatatcttaccccaattagaaatactccatgaccatagggtttatgtgttgagctagcgtcgtaggctatatatgtgtatccttattgtgtgcttgagctagtcaatcataatctctaataataaatttgtagcaactcaactttcattaacaaattggttgaacattgttaataaattcatttgtcttttattcttctatttattctcttaccctttttttggttgtttgtgctcaatattgtataacgatcgcttccattgcatcaacaattgataacaaataaaattggtagatagcttttaacccatacatcaatgaaaatgttaactttggattatggcaaagttggacgaaggacatgttagttatataatgcttgtcaatgtatccactgaatctgtcaccctattaaggttatatactccctataatattttattctcccctaaagggagcctacgcgactcgaagtctcgaacctatgacctcacgaactaatagtcattgacttcatggacttagtggtagcgacttaatggaatcaatgactataagtccatgaggtcatgggttcaagtcacagatgctcctcttggaggaataaaatattataggggtacttaagcttgataggcgacatgctcaccgaagggtcgcatgtcaaggctgaggtgctacacaagggtgtgtaacataggtgttggttgacttggttgaagggtgcgttggtagagatgtagccatgacacaagtattggttggcattgaggcatggcgtgtatggtggtaaggttgactaagatagtttcgttaaactataattgttattgtctacaatgattaccagtatcgacaatgatggcatgtattgatattttcaagtaagttataaggctcatctttgttgagtattagaataaggtggtacaagttcgtcaaaatactcagagaggcttacactcaaggaggggtggtctctcgtgtagaggaagtgtcatggaagatctggtaaagaatgttattcattgagcaaggtagagtgaaatattcactaagatggaaattattgttgtgcaaatttcctacgaagcataatattttggatgtggagattgttatatagattttcaaaagagtgtgataagaagccctagattgggatgcttggaataaagtatttaagaagtcctatattgactagaattggagtgaaggatagatgaagagtatatattaggttcaagttccttattttatcttctcgagttataaacattttgtgcctcacttatgtatcggatcagagtaagactcctttccattaatggagagggaagtacccaaaaaaagtcaaagaaagtagaagatgtaatgtcatttttgtcctttttattttttatgaaattatagttctaccctcaaaagtaacataaaaaaaaggaacttaggatctttttccattggattatggttgtaagctatagatatgtattatgtaaatcataaatgagtgtgcttgaaaatattaattctaatctctaataatatatcagtagcggctacatatttctgaagatatcaatgtaatgattgaacattgttaacaaattcttttatatttttcacttctatttattctatttcccttcatttaattgcatgtggttaacatttaactccttgtaccaataattattaatattaggcttcaatatattttttgttcttgaaaatttagcaaattttcatttggtccttataattattagttttatttgtttttgcaccaatgatataacaacatcataattagtcatacatgtcataattacatgttagccatatcgtagtgaataaaagtaaatttttgaaacatataagattcaaaaccaaatgaaaaaatatatatattaaaacacaaattcccaaaaaaattaaggaaaaatatatcatggtaaacaatatcattataataatataagagaatggtggaaaaaaaagtctgcttaaaacaaaagcaattatgtactaataaatacatattgtctcaaaccatgtcctatattttcattaatacgagacataatttaagaaatgtcattttattcactgttgtgataaccggatcggacggactctagttcatggtgttaaccgaatgaatggttagaacgctttgaaccgaataaatttgggttcgaccttcctaaaccaagttaaaccagaatagttctgaaagtaaaccggactgttgacttaaaaaaattcaaagtctacttttttattttagttcgatacaaaaaatacaaagtgatattacttcaacataattacataaaattattaatattataaaattttatatttatataattatatatttataaataaaatgtattaatcatatttaaaactactttttgataggacggtttatctgtttaaaccgattaatataaaacttattcagtttgatttcctgtttgtgttcacaaccattgcttttattatcaagatccatagg

SEQ ID NO: 109 - DNA, 3000 bp, Lupinus albus
  source 1..3000: mol_type = genomic DNA
tgctttttgtgattatattatattatattataagtgatacataattacttatttcaaaatgaaaattaatatctttctataaaattaactaacaaatccttttacacctatgatatagtttcacaatattatattttgtattgacttaccggtatcatatattactaacataatcaaattaattttgtaagaaaacatatcagttcctttaaaatttctacacacaaatttaagtagaacctatgtaattaacataggagcataaatcttaaacacaaggctaatatttaactagttaaaaagctttgtaaaacataatataataatatatttttatctaattactttgatatatttttatctgacgccaatacaacatgaattcaattactgttatgggagtaatcctgacgagctctattggatcataagatacatgtgattaacaacatagaattgaattaatcaaaaggaatatttaatatcaattggtatttaaactaataagggaaaacatgttttaacaaccaatatgaaataatttgcatttgcatttgtctatattgtttctcataataactatatttttataattcaaaaccacaattcaattggaaccaccaacaacgaataatttgcattttcttatatgaccattcatagtaactacacctctataacccaaaacaacatttcaattataaattgcagttgaatatattcaattgagataattgttcaatcttaataaaatgatacatatacaattatggtcatcaatcaataatagagtgttgcattctaatttttaaagactgcacaacacaataggaaaacaagttatgaatagttggtttactcagccccactcctccctatttaacaatctagaattgatttttcttttcctctatttccatgaaaatacaatacatgaatagtccttccatgaacaatcaaataaatattttatcatgtttagtttatttattacaaaaagttaccacatttgagggtagtgtatctatagaccaaattatattgtttcttctaatcattattcaattgttttccctatatgttttacttttattcctattcgattattttattttcattccaaatatttgataaaaaatattttagtggcaataaaaatagcattccttgtcaataaaataataagaaataaaaagtaggtattgatagaagaagagaactgatacccctaagttttataaagagtaggtattaataaaaaagtaggtataaaagagaaaataattttataaatagtaaattacactgacacaatctgattttcagtgaaattataaaaaacataactcaatttttaacacatacaaaaaactcctaatttataaatatacattacactcaccatctttcatgagaaacaagtgtaatatttaaaaaaaagtgataaatgtagttaagttaaacaaaatatgatatattgtgaaaacaaatccaaatattatacgagccaatatttttcattgtttttgttctttataatataatataagagttatttaaataatattttcttataattattgatgcatttggtaatgaaccaaaaataatgaaaatgtattgacttgtatattattttgacatgtctatacaatatatcatattttgtttaatttcattacacctatcatcatttttatgtaaaaatcatacttatttcacatgtaggggtattcattataatatatgtatataatttgaagtttttttttattggtgtaaaaaatggatgagtggtagtgtaatttcattataattaactcatttataaatttatatataatatatttcactaatagaattttgttatttttatcattaacaagacatttatatccttaaaaataacttaaaaaacaaattatcataatctttactgtgataattgtatcttattaataataaatataattacatataatagtgagtataaaatcatgggagtgaattcaattactcaaccattaagtttaaaaaacttattccatatcttaggtataaattttaaggaaattcaatggtttgaatttgtaatcagatttaaataattagatttatatagaattgatgtttaagatgtgaaatactcttgaaatgaaataagtaaatcaattctctaaaaatttatgtcccttgtctcaaactaatctcattctaaagaatataaaaaaattatttatatttttatgaattttttttaattaagtataatttttaatggaattgatacacaataatctaatcttcgttctgtaaaaggaatctcataaagttaagttattgtcgtgtaaattgattggtttgtacaagattgtattcctttcgtaggatagctttttgaattggtcaatgaaaaagagttttattcctttttaggattttattttgtgcgtttcacattttcattacaatatgggcagccaaacacagaattctctctaatattagagagagtagttattatttttgtaagataaaaatatcatgttattataaataaaataataaaagtgttataagatattaattttataatacctccttctctagtatttgagaatataaaccaatatcctttctaatacagataataatatacaatataaaattattttaaactatccatattttaaattatttttaagtaaaaaatttgaaatttccatttacctcatgatttatgatttagtactatacataattttgaagatgagatgatacttattcatgtttctcaatcttcaaaaacaataatatatattaataatttaaaattagataatatcaattcaaatcaagtatggatttagaactctattttaaatatcatgcaagaagatatggtttagattatgacacccaagtcgttgataatttctcttataatatataggagagtcctattccacattgcaatgaaaaacccttcactacaaaaaa

SEQ ID NO: 110 - DNA, 3073 bp, Vigna unguiculata
  source 1..3073: mol_type = genomic DNA
aattaataacttttctgtttttatcaatcattaaagaaatactatttgtgatttaaaaattataaaaatacctttaattaaaaaaagtcaatgatatgcctatacattcaaaaaatgaaataaactaaaatttgtgattgcaaaattgtaaagtaaaagcaaatgaggaagaatataaataaagtctatgaacaaattgaatatgctaataaataagtttaatatacggtaatacgttggcttttttctttttttaaatccgtgtactatggtattaattaaattaaatgataagtgaataaaagaagaaaatgtagaatacgcgtatttattatcaaaatttatatgtgtgacaagtatgttatgatttttcaattttctaaatctgattataccatctatagagttaaagtttatttttacttcttataattttcctgttattttaatgataatttttaaattttcttgttaggagtctcaattaaggcattgtgctcatggatggttcaagagtttataagttttaaagcaaacttaaaaaaaaaaatgagatctttcatttaaaatttaatttttaaagttttattatgcaaaattatttattaaattgtcatatttaatttcatttaacatctcttcttcaataagtaatttgacgacattattgatcttaaataagctcttatatttttagctttgagaaacttttaacattgtacttgtgaacattattttataagctatatatacatttgaaaaagaatctattatttttatatagttaagaatgtcaattagtttatcattttctaaacctataatttctcttaagatatttaattctgaaaataaatcaaattaatcaatatccaataaattatcatgttttaaagatttttttaaggtttaatcatttttatttaaaaaatgtacaatctagtgactttaattttttagcactaaataaaaaacataaatattttgatatgttttaaattgttcaaatctactttgaagtgcggtaattgttttatcttcaatattcaaaaggtaattgttttatcttttcaactcaacagtaagtttttttccctaaacttaaaatatctttctaaaaaaaacccaactttgaattatgatttaattaatatactatccaccaaaatactaacccgtaatctaaataatagttttgttgaaaaattttaatttctcgtcacgatacatacaagaaaaaaaaaactctcaaactcataaaacaaaaaagtattgaaaaataaaactggacaaaaggaacacatttataattttaatataaatccaaaactgactaaaaacaacactaactaacaaatagaaagaaaaaatataattttataattaatttaatagtagtatattattacaataaaaattgtgaggcgttttttcttaaatataattttataattaatttaataaaaagtattaatagaaaaaaaaaattgaaaccttatttatttggaggtctaaagtgaatatatgtgagatacaatttctaaacaagtttttttttaataacacaaacttttgattgaacaggtataatgttttagttaaaatagtagattttttttgttaaattcatttgtaaaccaatgtatcaattataacacataaaaaggttgaaataaatattatcctatcggtaatattaaatttttatgatatacataatcgtaaattgatatgtaaaacaataattctaaattaaatttgttttcaaaacaaattattgtgtggatttggatctctctgatccaaaataattcacattagaaaaataatctatgtaaaggaatctcatttatatttaagagtaaaagtttaataaatcaatcaagtttaatcttatcatacagttcatttctgttcatctcaataaatcacatttattaatttgttcctgtccattccttcactctctttttttgttgacctttataccaataaatgattgtataaacattgcacattacattttctattaatcccaatttgttttcttttttaaagatttgatttaataataaacattgcacattacattttttattaattaaccacaatttattttctttttgaaaggtttgatttaataataaacattgcacattacattttttattaaccacaatttattttctttttgaaagatttgatttaataataaacatttcacaatacattttctattaaccacaatttgttttcttttcaaaaggtttcatttaataataaacattgcacattacatttttttattaaccacattttcttttctttttgaaaggtatgatttaataataaacattgcacattacattttttttattaaccacaatttgttttcttttctaaaggtttgatttaataataaacattgcacattaaattttactgaccacaatttgttttccttttcctttttgaaaggtttcatttaataataaacattgcacattacattttctattaaccacagtttgttctctttttgaaaatttttatttaataataaaatgttaaatatgttttttatctcttaacttttaataaaatttgaaattagtaaattttggactaatttagtcttctaactgtagaagtgtataaatttagttattttaaccacattttattaagtttatttaaggtttcaaatatgtttcatgatattatttcaactaacattgaggtatgaggatatgtcaaacggtataaacaatttaaatactattacaaatgtatttaaaacatgaaataaaattaacaaaatttggttaaaatgactaaattcatgtatttttaaagataaatgactaaattaagtcaaaatttttaaaatgaactgattcaaattttcattaaaaatttagagatatgagaaacattttaagccaataataaacattgtacgttacaattttttattgagattctaaagtcaatcttcatgctctatatatatgtaggaggcaacactcaatattgcataaggaacgatcaatcccttgctcttccatacaca

SEQ ID NO: 111 - DNA, 3085 bp, Vigna unguiculata
  source 1..3085: mol_type = genomic DNA
tgaattattttcgagtgtttttatcatatctaaggcttttaatatatggtctataacaacgattccagtcacaattttatcttagattattgagctatcacacaagagaaaagagattatgttgaagactaaaaaaataaaaatatttactttaattttacgatttatatgaaggcatctttagagaaatttaaaagattatttgaatttttttattaacaatgtaaagaatttttttgtatcaaaaagtttgaaccagtttcgaattttaagattaaggtaatatgttacggggataagatgagaaaggattgtgtatattgtaggaaaatttttgaattttttttctatatttgatgacattgttaagaagttatttctcgagttaggaaagatggtataatatatccatttttataattaaaattctggaactattactattataagtaaagttaagtcgcaattcgttgtcgtcacaaatatatcatcaatattttcttttttttaatacataacttttagttataaaataggtaggtcctcttcataaaatattttcaataatatgttttacttttttaaatcacttgattaattaattcatttaatatattttttaatggccaaatatgtttgaatatttcttcagtatcaacataaataaaataatttctaattacttcgtgaaaaaaatattttatgtgaaaaaagaatcttaatgtctatgtttttctgaaaaaataatttatatttaaattcatgaaattaaacaattaaaaaaatttaaagttgttttagtttatacagtttgaaaaatacttatataaaagtattgtcaagagattaaatataacttaaattcgattatgtaatagtttaaaactaacacgataacatatgtgttctcatgtgttatgttttatgaaagtagagttggatggtaatttatgtggctcaatcaattaacagaaattcttagaacatggcacgtttaaacattaatcaaaggcattcaaaaaagggaagaaaaacaaaagacattaatgaagaattttggttccaccgtaagaattggacacatgctctgcctcgtaagagtaggaagctcatgctataaaatataatcaatcattcacactctccaacttactcaacaccaaagaaatccaaaattaataaagctaggtaaacctcaattattattcaaaagaaataaacaatattttttattaaatatactttttaattatcttacatcgtttaatatttaaaaaaaattgaattcaattgatctttgatctttggttacattatcatataatgtttaattttacgagcaggctatttcttttaaatattaaaagattacattatcacattagttttttatgcgtataaatgagaatggaagtactacttccgaagtagtaatatttttcgttttatatcaatggatcgtctatttaattttcttgtgttccaaaaatataaattggatcgtttgtgtagtactttggcttcaccacatatgtcaatttccagtcatgtgttgcctattgtactttattttttcctctgtttttcattcttgtaaagttatttttaaaatttgagattgagtgtagtttatctccacactaattatagcttgtaaatgtttaacactttacatgtccacacgaaaatttgtatttaggatcgaataagattaataaacaatcttcatcttacgaattttgtaaaattagattacattgataatgttagaatatcttttataaattatttgttgttgggtctattaaattactttttactagttccaaattgaatattaaaatgttatttatccatttgcaaatgtttaattttgcaatctcttaactctcgtgggtgtttgagatcaactttgactagtatacgagaaacataatgcttttaagagagagataataattatttaacaaaccaatggattaaattaggttcaaaagtgaattctaagatgatgctatattttatcttattaatttaatagacctctttattgaattatcaacaatctgcctacaaaattttagtctttgcaaactttgtaaatatttaatcgtgtaaacaaatgtgataatgcttatcctatatttaaaaaaacaaatttaatgcttacaatctatttatttaacaaaacaaagtttttatgtcgtttgtatattttttaatggaagataattaccaagagaatatgacaacaagtatatataaattagaagagtgttccttcactattacacaggaagaattatgcattacttaattaattcataattaattagttagggtgtacttatcaaggaatggaaataaaaaagatgtaacttgtgatttaataaataaaaagagagtctaaaaagaaatataagttcgttaaccaaacgtgtggggggaacatataatatgcaagaagcaatattattcaaagaaaaacaaaatacgcttcgtacgtacgtaccttgaaagaaaaattatgcaaataacaaatgatacgagttatggaaaatataaactatgtcagactaaaaagtaaaccaaaatcataatctgttctattaagcccaataatgcctttattatatggaaactgatattttgacatttttggatttcgttacaactacttagtgatttttttataatagtataataattatattattatatattcttacaacaagttatgaagtagttgtacttaaacttaaacatacacgttcttgtacacgttgtatcattgtctcaaattaaaaggaaaaaaaacacaaaaatttagctataaatatcaacatttttttcataatgattaaaagaataagaaaaaatagtaacacaagtgtttttttttttaatgaaactaatttaataataaacagtgtacagtggcagccgggcaaaattataaatttatagattagaatctcttggttctaaaagtcaaccattaagccaagaaaactcttatctttgcttataaagtggctgaggccgcggcaccacacaacgctgcaagaagcaatcacaaacaaca

SEQ ID NO: 112 - DNA, 3000 bp, Vigna unguiculata
  source 1..3000: mol_type = genomic DNA
aattgaaaaaatagtggtagaaataattcaaatttatagcttctttcatgatatattttttcatcaaatgatagttttactgctacaagtgaatataaatttcttactctaatacctctaataatattaaactttaatcattatatgtatttcaggtattatttattttgagtttaaaaatggaattaaaatatatctaaactatttttatctttatttttaactaatatgacactatttagaatatcagaacaatttttttccaatcaaatgttaaaggaatttttatacatatagccttgatattttaactttgagtttattgttgcatatgagtattatccactttaaaatttagaactcctgatacaattttctttttaaggaaggttttagaaaagtaaagaaaaatatatatttaaattttctttaacattattactaaaatgagagatatctataaaattaattggtaaacaaaatttattggaggtaggagtttaaaaataagtcttgtattcttaaaatttcataaaaattatataaaatatgtagaaatattaaatattgtttccctaatttgcagggagttttgcaagatcatgctctgattcactctttttaaaatattgctcttctatggagaccaattccttcaataaaacctattcatatatcagcctttcaatttttattattttttttgcacacgtactcttgtttaaaataacaaaattcaatcttgagtgacactaactacaaataaagaacacttcctcaaaaaacacttaatcaaacaacaaaatccattcacactttacattaaataatggtattttaagaaaattataattttacacttattacatgctcctcaatttattttttatttttgtctagatcacaagtataatttattcaatatggttaaggtcccaacaagtagtaaatatctgctgaataaaatgcaatcatatattcaaacttaatttcacttagtaagttgcatagtaactaagtactttttaatgtataataaccttaaatatatttattagaaatctaagataaattagaagtatcgcattaagtacaatataagtactgtgaataaaacataagtaaaacacctacaaacttattatccatgtaatgtatttttagaaactcaaacattatgagaatttgaaaagaaagatataaattataattataaatataaaacactacacacaagaagaatcaaattgtaaagtacataggttttggctttcactttgaaaaagacaaaacaatgaatggtattttgctttcagcatatggccactgtgctacacatggattcgacattcaaacactacaaagccaaacatagatgtccaacggcaatattctcaataattgctaaacttctaatactatttttaacttaacacgataatcaattatgtgatgtgttatacttagaatagttatgtaagaatgagtcattctgaatccgctaaaattttcttcctgctacttttcttaattagtatagttcacaaataaatatattaaacattttatgttttcaagatattaaatatcttttatagatatcaagacaatttatacaagaatataacattattccactataagagtttgtcgtgcgatccataaacaaaaactcttaaaatatgccatatttgaatattaaagaataatgcattgaaaagtggagaaagagagagaagtttaacaaagcaaagaaaaagtttccgttcggcttggaacttgtacaatccttggaatcgatcttccatgtcgtccaactaaactgccagcaaacacaaaccaacaaatgaaaactattataagtttcagaattaaataacttttttacacttcataactttactaactttgcttcttgttttttcgcacaaccatttaacaacaacccttatttgacgatttttatattaagtgacacaagaatctcattaacatgttaacttttttcctgtggttcaatgaacgtcttaattaattaataactaattaattaattaattaatgtaatatgtccgttagttattttttagattaaatatatttatggtcctttaatttttagttaaaatgaaattagttatttttaaaaattttgtcaatttaaaaaaatgtgtaaagttgattattttaatcaaatgttaaagaaatatttaagtttcaaacatgtttcaactgatatttaaattatttcaaatacatttaaatttatatgacatttctaaattaacattaattcagaaatggtagcactaaccgcgtttgaaatattttcataaaagttgaagttgattaaataagattaaatacactcatttttagaaattgaaaaattaaattagacaaaaatttcgagaaagactaattttaatttaaaaaaaaaacatatttaattcaataaaataacaaaaaattcatcacttaattgttttaagaaatactagaaatcaaaacaaaagtaaaagttatatatgaaaacaaaaacagaacaaaaatcataaaaaaaaaagatagaaaaaagttattggaaaaatatgtcttattcaatcattaacacatcacttaattattagaaagcgatataaatgaaaataaaaataagtttttttgttatagcacaaaatgataatttattttatcgaagtcatatacaaattctgcatttttgtaagagagaaagagtaatttaagcttgaattataacgtataatttacccagtcaacagcttccaattacgtcacatctgggctgggagtatacgaagagtgtgagaaggtaaatgttgttttcccattacaaaaccaaacacaaggattgtgaggcaataagtgtaataattattctaaagtcaacatggcaggtcaaagaaactcttagcttggttcatataagagagcacaaacattgcagcaagcaaacacatctcttcctcatcacaaaca

SEQ ID NO: 113 - DNA, 3119 bp, Phaseolus vulgaris
  source 1..3119: mol_type = genomic DNA
tctctgtttgtgctcatatctgttcttcgttttctctggttgcgaagttctgcattttcaaaggtacgtaactgcattgcccctcattctcttgtttccattgcccctaacaactgcattgaactccaatggctttcgctttccatattgctttcgtttttaatgggtttcgctctacattttgaaggtgtattgttccattgcttccgtcgctgtcgcatttcgctctcgccgctccgccgccgttgccgtgtttcctcttctccccttccactcttcaccaacaacgtgcccctaaaaccctagctcattcagaagaatatacaaatgcggctatttcaaataaccgcattcgtaaatcgcatttacaaatgtggctctatagccgcatttgtaaatgtaaaatagccgcatttgattagcatttttgcgctagtgcacccaattatctttagtatcataccattgtgacttagtttccacacttatcttgcttgccatagatgaatcttcaaactccattggttcataccctatgatctatcatttgatacaatttggtcatcatcctccatggtgataactccactaaagctttttttctatgagaaacttgatataggattgatgtcctaatgaatacccctcaactctaattttatggggaatttattgtgtccttctatataaaagaatgtacatcgtattcttaccactttttttatgcattcaacactcttgctaaaaaaaaggaatatcataaggctaatcacatactaatttattacctaataacaattatgagttttacttgtaaatattattgaacatgcccccaattatacccctttactaaaaaacatgtcatttatcttattctcttgtccaaaaatcacaagttggattgtccttaaaataagataatataattcctctcattttccacaaatccactccaatacttggtagtttaatttatcaaacattgttaggtctttgattcttctcgtccaactttctgtaagtcatttttcttcttccatctctgaatttattatcgtatcattacatttctcaacatggtcttgaagtctcatccttgacttccatttttgtgtagtagcttttgacttcaccactttcacatatgcaagttcgtcaatcatgtgttgtcttccatgttccattatactttgctttttccccttattttggcgcatatgaagttgttgtctttcaatttgtattatttgtacgcttttatccttaaggatgttaaaaaaattcacattctcaatcatgagataaagttctttttccattttcctttcatctctcacaaccttaaacctaaaaaattcaaatctttgtcctactttattctttcacctagatatgaagatctctcaaacctcatctcaagaatcttccacaaatcttcttcattgtaagcgcctacgaacctataaaagaagaaggtggacatgttttctcaaactcaccatatctcaaaaatatttttcatgaatcttttcactctaagcacttgagaacttataaaacaagaaggtaaagatgttgtttcagtaattctctcaacttttcttttcgctcgacctcgctcataatcgattactccccacatttttattatcttatttataattaatgtgaaatatttaaaaaaatagtatatcccatcatatccttaatattagtggatttaccatgtgaataacataataagtgatttaaaatatatttaaatattttcttaaaatttgagattgaatttagtttaactccataataataacatgtaaaatgtttaatactctatatctagttatcttcatgagaatttgtatttaaaatcttcatcttataaatttatgtgaaatttaattaaatttaaattcatgtgttgagataatgttaaaatcttttctatacattgtttattgttgttcaaattacatacaaatgtttaattttgcaatcactatgtcgataaatcttatcctgaatttgagagagggtattacatatccaacttagactgctatataacaaatatattacttacaagataataattatatatctaataaactaatggattgaatgagatttaaacttgatctctaagatttctttatcttactaattatttgaaacctctttattgattcatcaataatctacccacaaaattgtagtctttgtaaacttgacaaatttttaatcgtgttaaaaaatgtgataatgtgcttatcatatgctaatgtcaaacttgacctatactttatatatgattatcatacatctagttgttctttcatcttctttattatatctattaattgtttttctctctttatcttgcttgtttccacttatagactctaaaatgacattcagcattagtaacgattaaaagaataagaaataataagatcaacacattattttatttttaatgaaactattttaataataaacactgcacagtggcagccgggcaagattataaatttgggaagtgttgtgttaacttaacacttcaagatttattatcccttcttgcaactacttttattaatatttcaattaaataacctaattcaatattttattagtatatagaattattaaatggttgtaaatgcatgtttgaaatagtttttttctctaagggaaaaaatattttaacaccaatttttataaataatttcatatcaaaactgcgattaaaataataaattaataaattaagttaaaagtatttctttcttaacgttcatcttgtaattgtatatattactattttattggaatacagtattttattagaaaaatcagttaaaagtaatgtatttaatgaataagatatttttccttaactaagacataaattaaattttaataaatagttattattaagagttgttaggaagtggtagaagttttaagtatcactcttcttttcttcattataccttagaatttctcgtttataaaagtcaaccaatatgccaaacaaactcttatctctttgcttataaagtggcttaggctgcggcaccacacaacactgcaagaagcattcacaaacaaca

SEQ ID NO: 114 - DNA, 3019 bp, Phaseolus vulgaris
  source 1..3019: mol_type = genomic DNA
gattgtagtttgattgaatttggaagtgttgtgattgagaattggttgtttgatttaaatggttttggaattagaaatcatataaatgtataaatggacataataactcaatgattcaatgagagtgattgtatcatgttgaacaagaactgtcaatgtaatgtgtagatttataatttatgaattaaatgaggtattgatttataatctaataggtatattaagttatgcagaattctgtatatttcactcaagctagcaagttctagctcaagctaaaaaattatgggtgctctctggtggattttagctcaagctagcgaattctagctcaagctaaaaacttatgggtgctctctggaggattttagctcaagctagcgaattttagcttaagctaaaattctgggtgttctctggaggattttagcccaagctagcaactcaagctaaaattcttggtgctctctagaggattttagcccaaactagcgaattttagctcaagctaaaattctgggtgctctctggtggattttagctcaagctagcgaattttagctcaagctagcgaattttagctcaagctaaaattcaggttgctctctggcgaatttctagctcaagctagagtttaataataataataataaataaataaatgaaaataaaaatattaaactaatttttatttgcttttaaaagattgatttatttaattctatataatttttcttataaagtatgcaaacttttatttcatgtatagttaattgaaaatctctttaattagacatttatatgattagttaatgaattgttgtgtattttggatattctaaacattggtatgatttgattgtttgatgctggatattatgaggttcaaaatatgaattttaatcaatacatagtattttcaggaaagaaaataccgtgttaagattatttaggatgtgcattgactagggattcgtctagaaggagatatcctgactctactgaaataatggaatcatatagatgaagttattaaggtggtgaaagtcgaaggaggttcatatgaatgggtaagttgtttgaaagataactaacttgacctgttatatgagttaaccctgtcatactaggagagagatgatattgagattaattatgcgcatagttgtgtggatttcacagtgatgtagtatgacaggtgcagatctttgagtctaagtcaacgcacgagtcttcaagaagtacgagttaagtgtctatgtgttatgagtcagtagaagtctgacatgagaaagatgaatattgaaattgttgatagacacttgatggttatgtggaatgcatgagaaatgatggatttatgtggattttatagttatattatgttataagttttaaaaatacctagcttaccctttgttttgttttgtggttgtttttctttgatctgtgatgatcgtgtattttacacgagagcagatgatattacaggtgatcaagttttctcagtgagaagatgaatgatgaaaaatgtttattttcttttgaaattttgtttttaattcttttatgtaaatatttccagtcttataaagagagataatttgaaacataatatgaaagaattgtaaatatatttgtattattatatattttatttaatatttaatacaaattataattattagatatgagaaaatataggaggttacactatttataatgtattaaatattagttaataatttttttgtttatgttgttttaactatgtattacccactcccacaatagtttcttgattaaattcactcaacaattacttagtgatggtgtaactcaacatatttcaatatcttgaaataatattgtcaagattcttaaagatttccaattttctcaatatatactctccactcacgagctttattctatgttctgagtgatttttaactttacagtttgaatcattatctaatttgttaagtttgattttaatttctataaaaatcaaaagtttatttttatttcttataatgtccatgttattttaattatagtctctttaaattatttttatttcaatttaatgttatccattttaaccttaaacaatgtacacattttcatactctttttaataatttattcctttcctttttattaatttatttatttgtttattaattttattttataatttatcaattaaattatttaataattcattgaattgatgaatgcacaatattaatttatttagaaaaaattaagcttctttaaaaaaaaagtacgtataggaatttggtggaaggaccgaattttaaattggtttgtaaaacaaatttttgtatggatttagatggtaaaatggacttttggcccgctaacctgccaaattttgatatgctgtttttaaccttctaacataacttgtctcatctaactcgtcaaattgataggacaccactagcttatttttaaaattaaaaataaaataaaatattttttatactatattatttaaagaatgcaaatttataaaaacaatataacttaaatcataaacacttctataaattaattttaattattaattataacagattactttaaatgattattttaatttatcctcaattaaagtttttaaaatatttatataattaaatattttttaatatgtatatataacaaaatttaaaaatacaaaaactatctcagtattaatctcatttattaatctgttcctttcctttgttaattttgtatgtctatatatatatatatatatatatatatatagacatttgttctcctctatacaccaccatttgttttcttttttataagtgcaatttagtaataaatatggaaaattacattttctatttatatatagcaatatttattgagattctaaattcaatcttcatgttctaaacaatctcttgtatattaaggaggcaacactgaacattgcataaggatcaatcattcccttgctcttccatacaca

SEQ ID NO: 115 - DNA, 3117 bp, Phaseolus vulgaris
  source 1..3117: mol_type = genomic DNA
tttagttcacaaaaatgtcatacattcaaagaacacatgggagaagatagcattggagcatagataccacaacaaaattatttaactcactcaccaaacacatggcccctatatttgtagcatagttgcttcttgtaggtattatacatgaactattatttttggcttttgggtcatacatggctgctcaaattattcacaagcttgacttatgcgtcaattccaggatctggacagcttatacaaaatttcataatttaatactttttttaaagattaatttctacatttttaaaagactagacaacaattacgttaaattaatgaagtaaaacatattcctgtgatacaatcaagcttaaagttactaatatttttggtgatacaatcaagcttaaagttactaatatttttggtgaaagaaatacgtgtgttgataagttggttaatttatgatttattaatagagaatcatttcattggtataatagacttccatctagtatgttcttagaattctttatggataagtatagtctacctatgtatcgtttttgttaacgtatgagttttggtctagtccccctcatatttttttatttttttattttctttttttttttaataatatttttttcatgtgatgacagatgattgttgttacttgaggtgtcagccttggtaagatgtcaagttgtataatgatgcctaacatgaaaattttttataaaaaaaaataaaatatttcaataacttgtgattatttttcaataacttttttctgcattagctttatagatactctttttatatacaaaaaccaactttttcatattgaaataaattaattatcttgtaattttttttactgaagataaacgagtattaacggattataattagttataataattcatttttacagcgtaataatgacatatactatgtatatagtgttctaattgtaaaagcgttagtatgtggaaatgctttaaaactattaaaataaaaaataattgacacaattgtttatatgtaatttttttttaccgaaatgttgttgaagaaaggaaatgctgtagaaaaaaatatacacaatgaattactgaaacaaattaacttttactaatataccaaacaaaacctaaagaaagaaaagaacactctttgcaagtgcaaagcacgttattgaaaaagcaagactttgttgcttttattgaaacgtcagtattgactaaaactgaaaatcaaacatatttcaaagcaattgcagggaatttaagaaaaactattttttaaaaaatttaaaataatagtttatttattaaaaaaacattgtttattattttttaaagtgtttcttttgaaaaaaaatataattgttttactttaaactatttattaacaaaaaaattaattagttaattaattcagggattttgtttgataaataataataaagaaagttcaacggtggttttttattttgtagtttgctttagaatatgtaaaagcacttttgtctgtgagttgaacacatttttgtttgttgtttaacggtcacctgctacgtcttctataagggagcgttgtaacgtaaaccatcgatatttgatttcgtacggactacgttttttatttttcatttttaattgaatatctcttttaatttaatatttcaaaaaataaattttcaacattccctctgattttttcttaatttcttaaccagtgttttaagacactactcaacccgattcataatattatattataatttaacaattaatgaagagttgaagattattggaataatttacattatattagttcaacggtcatatacatcatgctgcatcttacttacaacaactacttatttatttctctctacacaattttaatttgatttttacttaaataattgtttatagattttcaaagaagaatacatttaaatatatttttgctaacttgaatttttttattttcgtctagaattttttttctagtcctcacattcaatataattattgagtttttttttttatccttcaatatatcctcaagtcgatctaaatatacataatttttgcaatttctaaacactagaaataaaataaacactgaaatagcaacattaaatctacaactccagaaaaaaaaaatacttaaccctagctaatgaacaaatttttttataagaacaacttaataagatgatcagataaaagttcacaatgattagaagaagaaaaagtaattcaatgacacttttagctgatatcaatagttaattatgagtaaccaattatattatgggttatactttaaaataatagttagataagaataagtctttctaaaccctctaacaagttcttcctgctacttttctttaattggtatagtagataaataaatatattgagccatttatgttttcttaagatgttaaatattttttttatcgaatattgtttataaatattaagatgatttatacaagaatatatcattgaatattaaacaatacattgaaaactacagagagaaaaagaggtttaacaaagcgaagaaaaaagttcctcaatttgaataagcttggaatccatcatccattattttccaaccaaaccgctgccaaacacaaaccaacaaatgaaaaaaatacacaactatttataatttatagaatgaatgtttacactttatagaattattaactctgcttttcatttttaatatttttgactgaagatttttctattaagtggcatcagaatataattaacatgttcattttttttcttttggttcaataaattttatatgtgtgtgtatattacacatcacttaattattattgtattgacagtcatatacaaattctgcagttttataagagagaaaaaaactatttaagcttaaattataaatgtataatttacctactcaatacttaattacatagctttgaagtgtgagaagataaatgtggttttaccattgcttaaccaaacacaaggattaaaaagcattattctaaagtcaagttgggaggtgaaagaagcttagtttggtttatataagagacaacaagcattgcaggaagca

SEQ ID NO: 116 - DNA, 3068 bp, Glycine max
  source 1..3068: mol_type = genomic DNA
gatgacaatattattctttcttttatattgatttggttttgatttattttgagaaattaatgttaattataattaatttttgtctatgagacaaacttgcaccatatgaaattgacaactcttctaaaaagaaaaaaaaaactttaattttgagacatatttttgtatcagcctttaatttaagacatactaattaataactatttttcttttttattactattattcattttttcgcttatcctatttttattctttttgtcttgttagctcaaatctaatttgtaaaagcaaacttaatattaagttgtaccaagtatttgtcaaaatttactttaataggttttgagtttttttaaaaataaatatttaatgtaaatattttattataaaatcatttaagttatagttggtcctgctaacttttttttgctaggtctgtctctgctgcttgatacctactctgacactcatgtgctccaagattcaaattacctaaatttttttgaaataaaaaagccacatccagttaactagcatattactatctaatgctaatgttctcgtgtgtgtatatatatatattgagaacaagtagttccccagacgaacagtaaaacaaacctaaagctaacataaggtctacaatcaatacttatgtccaaatattctcaacaaattagatataacttaaattcgattatatatgtagagtttttttcaaaacttacataataacatatgtattgtgtcgtgtgttatattttaatagagtagaattgactaagaataacttctcgtgcaatccattaacaaaaaagtacttcatctgttatattcaacattaacaaggtagttaaaaagaaaaaaataataattaatacacaatgcaacgaacaagttttgttccaccgttggaatttcacgtgccttggaaatggacacatgctctgcattcctaacgtagggagcttaattatgcaataaaatatatataatctgtgtaacaaacatgcagcaaagaaattcaaaataattatctttcatttattgatttatctaatataaaaataattaccatagagtcaggggcggacccaggattggtggaaaggagagccaaatatgaaaaataaaagaaaaaattatacctatcaattgaaagatttttttccctcaattttagcaatttttatttttaagttgtttataaaataattatatattcaaaaaaattatttacatcaataatttactaaaacattttttttacatgtttggaaatcatctataatttttttagtaaaaaaaaatcatctatggcttacacagattttttcaacaattttctttcaaaaaaatatttttctaaacaaaaaaaaatatataacactattttaacacattataaataaatttaattaaataaaagtatacaaattgctataactaaatttaaatttatttttgaagatttaccggttagcctatctagacacgtgcaatcgaaggtcaaaagagcagtgtgtctcacaaacataatgcctagaggacaaaaataagaagaagaagaaataaagaaaatatatagagttatcataaaaaaatagaatatttgttgttatgtttgaaaatcatgtatctcttatatttgtgttaataagcttgctctacaatttcaataacaaaggtttatatataagtaacttttatttctcaaattaattataatcaaaaggctaataaagctatttgattgtaattataatcatttatagtatttaaaacaccaataaaaaattataaaaagaaaattacgaataaaaatgaatgatactcaatttaaaaagagaaaaaaaataacattagttcaacaatgaagactataaaaagaaaactataatatttatctaaaaattgaaaatggtcattatcttcatcataaaaagaaaattacaataataattattacaataataattatttaaaaagaagatagattcacatactccaagaataattcttttatagttatttcttgtctttattgtttattttattgaaatctaatggttaagattagttatttattacaactattaaatttaaaaaaaaatgaaagatgaggataaaaagtaactttaaaaaagttactctttgaataagttgattcctcctcatttaaaaaatatgacattatttttataatgaaaactataaaaaaatatatcccaatatatatatatatatatatatatatatatatatatatatatatatatatatatatatatatatatatatatatatatatatatatatatatatatatataaaaatttacccctatacaaaaaattaaaatttagaaaaactaaggtggtacaaaataaaaatggaaaaatataaattaaatgtctaattttaagttataagaaaatcatagtttactctcgtagggtcaataatgcattcactatcatactcttttttggacatttttatcaccaactaaaattgattaaaaataataaaaatcataaataatatttataaatatcatacatctagtttctcattaatgagagttggatctaataaaagggttaacctcccgcaatttaactaatcaaggtttggatcctcatattttttcaatttctttcttaagttcccattacattaattacaatattatttatcacatttaatgttttctttttttttttttctgtctcattctttcttctactcatacgccccataagtggaatgtagattaattttttccgtactaattcgtagagtaagacatatgaccaacccaatttgttttatttttaatgaaattaatttaatagtaataaagaatgtttaggggctacaaccggacatggttataataattattaattaataaatttatagcatttctcttggttctaaagtgcaacctttggtaccaaacaatctcttatcttttcttatatatataagaggcttaggccggcaccacacaatactgcaaagagcaatcacaaccttcacttccccacaaacaagaagca

SEQ ID NO: 117 - DNA, 3000 bp, Glycine max
  source 1..3000: mol_type = genomic DNA
tgtaatttcatggatcttagatgaaactcatttttatttcttatatttctaaaaaaaaattaatttggtccaagtaatacatcatttgcattaatttacattattcttatatcgattcaataaaatgattaatttagattattttttattggtctttgaaggtaaaaatattatttcatgttatccgaatgttaaagaatatttttatataactacttttattaataaattaattatatttaatttatcactaatttagtattgaacatattggattacatacaaaagaatgttttaatctttacataagaaaataatttatttgataattactttgaagaacttaattaaatcaaataaatattgtagtagaattaatattgctatattattttttttccgtaggaattgaaaatgatattaagactttacaacaagcacacacaccatactcaatcaattaagctagatcatttgatatattgttacattctttgtcagcaccaaaaatatataattaatattaacataaagagtgttttttcttaaatgcagaagttatccataaaaaataagaatcataatctattattacatattaggattaagtcatgtgattagctaccatagttaatcatattattttttggaaaggccttaaattatgaaggttcttaaaataaagagagatgaaattattttaagtcaaatatttctcctaaaaaaagtctaaatccatatctctttaataccataaatagaagtatttgactaaggattagtttgatttataaaaaatatggtaatagacaagataaatatttttggtctaagattgatttggaaaaaaagttagggaacaatacaaatcaagtagatgtgcacgagacaaaatctctaatttttgttactaagaaaatcatcaatcaattttttgtcttaggtacaaattattcaaaagatacatttatgcttatttttatctatttaataactaaaaaataattactgacttatataatcatttttaaaagaaaacacttttaaatctctctcacttttatttcaataaacacattaaatttgttaaaatatagtactaataaggataataagaacaatatagaataaaaatacaaattacatgacaattttatctagtacatcggacacaagttagaaaagtttaataagagtctaatatctaaacttgaaaagtagaaagataatttgttattcttagaatttcacttattaaagagatgataacaagatatttatcttaatgtgaatgcatgtagaatcattttttttattaattcaagaacacatattcaaatgcataaaatcttttatttatttattattgttgtaatatattataatataaaataaatcttattattttggtgcaagaattagaaacactccattataacgatcacatttattctaattaattgggtcaataatgtctatatatcacattctctcttatatcttattttatcattgaccaaaaatcttagaaaataaaaattataaataaataaaaagtttctattatcataggtccggattttctttcatctaattaattttcactaattttttaaaaatatttttctatttttttattatatcattctcctatgcatttccataacaattaattaacccaattagatgtatttttcatgaatttcttttgataacaaaaagactgtataggagcatgattattaatttcaaattatagcatttctctcgatcctaaagtcaatcttcatgttctaaacaatctcttattttttcttatttttaattttaatatctctttctttcacccttataaccaaaaatggggtctaataaacgttttcataataattcattgaataagaaatatgaccaacacgatttattttattttaatgaatttcttctaataataaagactcaataggaccatgattatacatttctaatgctaacatttctcttggtcctaaagttaaccttcattgtttttcctgagtttgtcatccttcatgttctaaacaatctcttatacacatggaagagtattttattatgagaggtgagaggattaaatagtcatcctgagaacaaaatagaaggtcctgatttaattattaatcaaaattacacgtgcatttaatattggatctatcatgtgcaatgtttttctcagtcttggatttacaatatagttttaattaataattaaatttggaccttctattttgatcagggttgttatttaatcctctcacctctcataataaaatactcctcaccagatatgccctctatatatatatatatatatatatatatatatatatatatatatatatatatatatatatatatatatcattttttgttttttaatcattctcttacacctatatcccccaaaatcaacatttccttgaataagagataaaaccaacataatttgttttatttgtattgaatttcttcctttaataataaagatcatgattataagtttctaattctaacatttctcttgatcctaaattcaaaattcatattttaaacaatttcttatcttttaaatattttttttcatcatatcatacctattaaattctttttcttctttaatttttttacctttatttctctttctttcacggtttcgcctatatacccaaaaaaatggagtgtattaaacattttcataataatccattgcataaaaaataagaccaacacaatttattttatttttaatcttaacaataaagatagtataagactatgattcaacatttctaattataacattttctctagtcctaaagtcaaaattcttattctaaacaatctctttttttttatttttatatatatatatatatatatatatataataagaggcctaggtcagcagcaccacacaacaacactgcaagaaactacttctcccaaactcaatccacagtc

SEQ ID NO: 118 - DNA, 3550 bp, Glycine max
  source 1..3550: mol_type = genomic DNA
aactggttgatgctgattataaaaatatttttaaaaaaaaattatttactgtgggccattgccaacgctaaatgttttgctttactgctaatttttgaactagtgttgacaatgaaaaaaaaatatttaccgtggacaataactaagctaattcttttttccgctgaaattaagaactaagatcgagataaaactcaaataaagttgctcacactttaccttcttatacatttttattttaatttattttcatcattgctggattgaagaggtccttttcaaatggtctcggtgtgaatactttattgcttgaataacaggcattcagaaatactacaagggttagaaacactcccttataactatcacattaattctaattaattgggttgtcaataatgtattttctttttatcacattctctcttatatgttattttatcaaccgctgattttggttttggaatttacaatgatcaccattaccattggggaacttcctctatggaattgcggttcttgcaaagattgaccctccatggggacaaaagtttattcacttgcgacagatttcatgaacttgggccaaaagtataacagattttatccacgtctaaggtgtgttgaccgagtttgaggatggaaggaatcaagaaagtacaagtgaagctgtgaatgcatactattcagcagcattggtgggtcttgcatatggtgactcaagtcttgttgactctgggtcaacgctagtggcattggaaattctagccgcacaaacttggtggcatgtgaaatacaagagggatagtaagctatggtgggccagtgctgagtgtagagagtgtaggcttggaattcaagtgctgattatgccaaggaacttgtggaatggacatggccttctgcacgtagagaagggtggaagggaatgacctatccttgcaaggaatttattataaggaaacagcattggaaaatataattttgttgatgggaattctttcactaatctcttgtggtggattcatagtagatgaggactgatggatgcacatagttttgtgacaagtccaggaaatatcatgtttcaatgttatatttaatttgcagtaatttatgaaactttgtatcttctcatcaggttattgttattatggtggtttaaactttgaccactgcataaagatctttagagtttatgcaaatgtaacgcaatcacacatgtaaaataaagattactccacttcataaaaaataaactcacaagaataatgtttgatttctgctatccacgtaagaattttatcggtaaataattaaaatatctttataagcactctttaatttttgagatatactgatagatactgatgtatttcgaaatctaaattatctaaactttttaaaataaacaaaaaattacatccaactaatgtattactatcaaatgctattagtgttctcatgtatgtatgggataagaaactcgcatataatacatattgagaaaaggtgctaaaatgatcgatcaaactcgttagtttaattaaataaatgagtcaaattttaatcattcattttttcacaagttaaacttaattttctagtactcaatttggttcatttagaaccctaactaacataatgtctacaatcatacttatgtgcatgtattctcaaaaattagatctaacttaaattcgattatacatgtagtattttttcaaaacttacataataacatgtgtgccatattgtgtcgtgtgttatattttaatagagtagaattcgataagaataatttctcgcgcaatccattaacaaaagtacttcaaacctgttatattaaacattaacaaggtatttaaaaagaaaaataaataaatttatacacaatgcaacgaaaaagttttgttccaccgttggaatttcacgtgccttggaactgggcacatggtctgcctcctaaacttaggaaacttatgcaataaaatatagtcaatcatcgacaccctccaagttaataaagatgcaacaaataaatccaaaaacaataaatgaaagataatactatatattttcatttttttatcgatcatttaaaaaatatttctaattattcattatttgaaagtttctaaaataacattaattatattttttcaattatatccttgttaaaaaagaaataaataaatgatattaaagtagtaaatagagagagacaaatgacatattaaaaattgtaataattttgatataaacaaaactatcaattattttttttaatctatgtgttgtaatcttaaatgataatattaaaaaggaaaatttattatattaaaaatacattaagtacttaatatttttttcagtaatgtagtacatttaattaagaaaatatacttgatgctccgaatctatttagttgatgtataacagagaaaacttagtttttaatattattattatatcttttaaaggaaggaagaattaccatatatagagatgtaagactagtgtaaggaaaaatattaaaagcaaagtgttcttttgttattatttatctaggaaaaataattatttaacattatttaggttgtatacacatataagagagaaatagaaagaaaaaatatatgcaacttgtgatttaatgaagaaaaaaaatgaagacaaaaatgagctatataaggttgttgaccaacacatgtaatgcaatatacaatatgcaagaagcgttattctaagaagaatgaaagtgaaacacccttcagatccgtgcaagatacaatatgcaacaagcattattttaagaaaaattaaatattccattgtatttttataccttggaagagaaatgaaatgaaattatatgcattctcattataagaatgggtcattcaacactatttaaatagataatacgtttatatatagatccctgctgcgttgtttcattctattttaagattaagaatagacataatcaaacatatttcttaacatatctcttattatatatttagaacattttggaacaagaggatcatttataatgaaaaggatgagcttctaagttcatcccagagtttcaagaaaataatgcagagcttgaagaaaaaaagtcaatctctctcggtgtaactaacaagatttcgatcttttcacgtatcttttctatttttttcttcgatctctatcacattaattgatttctttttttatctttgtctccatctcattgtttcttctactcataggctctccataagtggaatgtagattaaacttttccgtactaattcgtagatcgagtaagacatatgaccaaaccaattagtttttatttttaatgaaattaatttaacagtaataaagcatatttaggggctgcagaagggtatagttataattaattaatgcatggcatttctcttgtttctaaaagtcaacctttggaacgtaccaagcaatctattatcttcacttatataagaggcttaggccggcaccacacaacactgcaacgagcaatcacaaagcaacg

SEQ ID NO: 119 - DNA, 3029 bp, Glycine max
  source 1..3029: mol_type = genomic DNA
ataacaggccgacaaaatggcttccccccacatttgggtagacataccagaacttaataacatagcgttcatcatttctagttattactactttgtcagcttcaaaaaccaattttaatccagctctatttagacaactaccagaaattaggttcctacgcagagagggaacatacaaaacatcactcaaagataatgtttttccagaagtaagtttaaggagaatcgttcctttaccaagcactccagctgtagctgaatttcccatgaaaacacattctccgtcatcagcatcctcaatttggtgaaaaagctctttgttggcgcataggtgtttagaagcaccggtatctagaatccagtccaccttgttgtcgaccatgtttgcttccactaccacagcagctatcacttcttcattttctgctagatgtgcttgaggagcatgtggtgcagcaagtttcgagttgttagattgttgtcccttcctcagttttcactcataggctttatgacctgttttgccacaaacatagcactcaccctttcttttctgaatcttgttatcacctttgttgatattagtgtgtgttttcttttgtccttttttctgaaatcatttttcttttccttttgacctgtattgatcagcaaactcaacaacattagcattaactgaatttacagaatgtaaaacatgcttattttaagtcggttggcctcctctgtcttcatatgatttattaattcttgaagtgacaagtccctcttcttgtgtttctgttggttgtggtagtcactccacgaaggtggaaatttttctagaagcacattagcctgcaatatctcacacatcttcattccttcatttagtatatcaccaactagattctcatactcatgaatctgctccatgattggtttgtcatccatcatttggaaacgcagccaatttcctaccacatatttctttcttccagcatcgtcatcgccgtaacgtttcagcagagtgtcccatattgttttcgcagacttttggttaatgaaaagatcaaacagattatctgccatatgagttagcagatgacctctagcagttttgttgtccttttcatatttcttctttgcttcttcgtcagctttcttggtttctgcaggaagcggtgtgacgacaggggtagcaggagtttcaggagtagatgattcagaagcgttgacattagcgggaggatcttcaaatagcacataatcaacctctaaggcttcaaacaaaattaacagttttttagaccatctcctataatttgagccgtctagaggttctattttcgacatatccggaatgatttttgatagatttccagccatttctgtttagtacttgatgcaatgaaaaaataattttgttttgctttcaaatttgttggaaaaactagaagtcataagtacaaaacagaaacatataacacagattggaggtttgttgtgccgtgaaaaatcactgccttgaaagcaaaattcggctagaccttgatgcaatctagtttgccgactgctccccaaggttaacacaacgagcctatgaacacgtgcactcgtggccttaggctatggaaatcacagaacaattgcccagaattttagaagtagataataagaaaagaattggaaattattttctgcctgtgcaagcctgtgcaagactatgacagtcctctttttaatagcaaaaccacaaaaacatctttttcttttcttgtccacaaatcataattattagctacacatttaacaaattgaccgttagagaattcctaataaagtggaggttttgctatttgatttatagctaatcaagagcattaaaacaaaggaaatatttgttaaaagaataacaatcaaaactgtttaaaatcagttaggaaatttgggaataaattgtggcttcagtttgccattgtccaacagggctaagagtgaaaacgaagatattgaattagtgtggatttggataagtgttataaaattaattttatttaaaattgattttgaagtgatgtaatttatatttaaatatttttattataataaaaataatttaactataaaattaatatatatatatatataattattttaattcaaaattaattttaaaatcaattaattttgattttttaaagtaaaactaactatgtaaaagactatttaaaatatatatttttagcataaaatcaaatacacattttataaattcaaattaactcatttagcctacactataaaacaaaagtctctatatcaatatgaacaagaaagagattattggataagagaaatatttgtaacatttcattcttctgactatctttcttatccatttttagtttattaaaatttattaaaaattataaattttaatgagtctcatatatcatttaatgatattttctcttgatttaaatataaaactcattaaaatttattattttcaataaattttaatcaatcataaaaatcatctctaacattcccccgctgaatatgctttctatagtaacacaagccctaagctaatatggtaaatcagagggagtattcacatactagaaataaatttagtctcttaaacattaagagaagtaaaaaaaagagtgtgtgaaaaaaaaatctcaataaattagttaagatatatttatatttaaatctaaaataatgattcataaacatccttggacttcaaacactacattaatatctttcatttttctcttctctttttatctattcataaacattatcatttatatttttctcttcttttttttcaagtgttaaatggcatattgaagtgtccatccaacgtttccttacattaaaaatcggtgaacccggaagtagaatttgtcttggttctaaagtcaactttcatgccgaacaatttcatatctttgcttatataagaggcttaggccggcaccgcacatcaacattgaaagtgcattcaaaactccaatcacaaacaaact

SEQ ID NO: 120 - DNA, 3039 bp, Glycine max
  source 1..3039: mol_type = genomic DNA
aaattttaaaaatgttaatgttgtcatcataaattttttttattaatttaatttttgtatcagtaaaacatatttttgttagttctataatctcgttataataaatttaattactttctctatttatatactcaagcacaattattatactaaaatctccttgtttcatttttcccaactctcatcactacatagtatgagaaaattagtgaaaaagtgatactaaattataattttttgggatgtttacatgtttgttaaatttattttggaggatttatctatcacaaattgttcattcatttgtaatcaaattacatgtgtaacattaaattaccatttaatagagttatcattcactataagtaaaaatatgttttttttcatcgggaccaaataaatataaaaacttttatgagtataaaattaatattttaaaattttaaaaacaacccaaacatattttacactaatatttaatgcaaacttttatcatgtatattttttttactgcaacttttatcatgtataaagagtttttaaaatatcatttcatcacacattgtcatgtacttttattcatcgcggtagaaattggattcaagctttatttcattgcacagatttatatatatgaacaagattaattgagaacataaaaatttattgcagtgatgatatataaaaaaataaactcttatttatttcattctacttctttttttccccatcaatttgaacacaaccttattgcttgtcttaattttgaatcgagattctcccaacaagctatttttcttatcacaaaacgagttacatggaagtaaatgccttaagttatatttttaaaaaataatataaagtatatatttcaactttttattcatcaaaattgtccctaatttcataattatcatacttttcatcaattaatagttttattactacaaaaatgctcataagtttcttaatttataatataaatttgtttcatctgttggggatgattccgagttttctaattattagtactaaattaagtgcattcatcaatacatcatttttttattagaagcaaatgggagattttataagattaattattttaattaaattttgtaggagctaagagctctaaaaaagtcttatttttgaaattgcataaagattatgcatgaaaaaggtataataaatagttttagatggatttacattgcttctttcctttgggggaaattttgcaagaacatgtaccaattcatcccctttctaaggatcactccctcggtttttttataagtgtcgtttaagttttttatccgatcaccaaagaaatttattactctgcctgcaccgttttgatttagtgtaattgtgagtactttttctatcatgcccttctctttacttctcccactaatgcacgtttttggaaggtggtactagtgcttttctaatcaattgctctctctccattacttgtttaatacttgtgggtatcattaattgacaattggttcacaacatatattattataaaaaaattggttggaaatattaatgttttttagaagatgagaggataaaaacttttgtcaagtaactatccactaaagttttgaccaataggtgattaatttaccttttaaaatttcaaattacttattatttaatttataaataatatgtgaaaacaattaattaaaaaaaagataaaatagacaaaaaattaatgttctcttgaatattttgttaaaatatgacacttacaagagaacggaaggaatactttttaacgggttcaaattatagatatatgcatgaattaaacaaaaaatataaataaaaattaaaaaaatgaaagaatttgtagttaaattgaatagtacaccattttcccaatgcatcttatgggtttcaacgcacaccttttggtctttggtcaaggatttcagcttttaatttcacaattattacagtcgatacatatgtgaatttatgtattgtagctgaagtgctaatgggggtaatcacaatcaatgtgatttttattgtgttttcagcattaccattgtgttacacgatcaccacttgcaaggcaagcactaccgtagctttcaagtttcaacatccaaatagacaaagaaaataaaactgaacggaaaagcccgcatagtataaagtccacaatccaaaaagagacttgtttacaacggcaatattctcaacaaatatgtactataataacaaaattaccatatacgccaacattacgctgtgttatattttggtagaacagagttaggcaagaatgagaaattttaaatcctcttccaacttcttcctacaacttttcttacttaatttgtataaggtattgagaagatttataaaagaatatagtactaacattgttccataaaagctcttcaaacattgaaaactgaacacaaagagacagaggggtttaacaaagcaacgaaaatgtttccgttccgcttggaatctttacctgccttggaaatggtcacatgctgtgcctccctggcatgaaaaagatttggaaataaaacactaataatcaatcatcggtatcctccaagtaaactgcatgccagcttatacagcaatattacagtaaattataagttaagtggaaaatataaacatttaccactcaacactaattgcatagctttcaatgaacactatcttgtactatattcctcttcactctaataataagtggtgagcataattattttaactacaaacatttacctactccacttaattgcatacctttcctttaacactatcttgtattttcctcttcactctaaatgttttttttttaccatatcttaaccaaacacaaggattctgaggcaagatataataatacacagaagcattattcaaaagtcaacttgcaagtctagaaaagctctcagcttagctcatataagaagagatcacaaacattgcaataagcaaaccacaaaactcccaacccttttctcttcccccacacaaaca

SEQ ID NO: 121 - DNA, 3040 bp, Glycine max
  source 1..3040: mol_type = genomic DNA
tatacatcaactaattacattttattagttcaacggtcatctatgatgaaggtgcatctccactgactaaacaactcaacaaaaggtgcatctatatgaagatgaatcaaaatagttattcatagataaaaataataaataataaataaattatattttcattaatttaaaattaacatatttatcttaattttttttaaaaaaatatctcatccaactctttaaaaattgaaatacgtgatttgattttaatgagagaagtttagtttgttttccctccttatttcttgttttataagtatacttgaaaaaaattatccaatctgaaccgtagtcaaaagaaatctttttttctaaatgattaagattatgaaacctctttaagtacttgatcaatattttattcaattttatgtacgttttctatgtggcatatatcttgggataagagtttaattttaatttatcatatccagttttgtcttttaattaaaaacatattttataagactttaaattttttctttcataataattttactcatcttaaatacgattttcttccttgatactataaaaaaactttaaaagctttaaaagcatattttgacataccatttaagtaatctaaatttatataaatcgaactaaactttgaataagtcagcaaatatatagtttcaaaatagtctcataaaatttcaataaaatggtaatagtagcactaataaagctattattatgttatttatatttattaaaaatgggctgcttgaccttctcctatcttctattacaatttaaataattaaaataatgagaaaacatgtattacgatcatataaaattttacattgatcagaaaaaaagctatcttatacataataaaatttagctcacattgaataactagaatataataaagaaaaaattttagatgtaatgttttaactgacatctgaataagtacaatataataaaggataaatttcaaatgtaattttttaactcacatcaaagttgtcccaaataattttagaatttaatgtttatgcattgatgtgtaaaatagttttataccatcattcgatctcaaattatcgtttaaattatttaaaaataattactttaaaagtgaataaacttatcatataaaatgaattgtgattgaataactatataaaaaattttacattatacgtgcataattctttttctcatggttttattactacaaaaatataagcttcttacttataatataaagctgtttcattagctgggggtgattactggaatttctaatatcaattactaaattaagtgcatttattaatacatgctttttttattagatgcaaatgggagataattataaaattaattattttaattcaattttgtcggagctaagagctctaaaaaaagtctaattttaaaaattgcattaaaaggtataataaatagttttagatggatattcatttgttctctcctttgggggaaatttatgcacatcttgtgctgaaattaaaaccgcttacaaaaaaaaatgtaccaattcatccgctttttaacgggttcaaattatatatctcccacgtatgtgcaggaactaaaaaaatatataaataaaaactaaaaatggaagaatttgtagttgaactgaatagtactacaccattttcccgatgcatcttacctgttttcaagtgggaaagacatacgcattttttttttaatgaaagtattattcaatgtcaaaaattaatattgaaatttattaaagaagttaacttcctccaataaatattttgaatgtgttggtgaaagagatatttagttgcaacttgcaaaattggagaaatcattcagtgttgtaaaattcaacgcacatctgggtcactggtccaggatttcagcttttaattccacaattattagaatcaacatatgtgaatttatgtgttggagctgaagtgctaatgaggtaataataataaatggtcaggaacacaatcaatgagattttattgtgttttcaccattaccattgtgctacacgatcaccacttgcaaggcaagcactaccgcagctttcaacatccaaatagacaaagaaaataaaaagtgaaaactgcacggaaaagctggcatagtaacaaaataaccatataggccaatattatgtcgtgttatattttggtagaacagagttagataagaatgagaacttttaaatcctcttacaacttctccctacaatttttcttacttaatttgtatagtataagtaactaaatattaaatatatttattttttttataaagtattgagaagatttgtaaaagaatatactagtaacattgttcaataagaatatcctgtgcaatcaattaacaaaagctcttcaaacattgtaaattgaacagagagagacagaggggtttaacaaatcaaggaaaaagtttctgttccgcttagaatttttacctgccttgggtgttcacatgatctgcctctctggcataaaaaaattatggaaataaaatactactataatccatcatcggtaccatccaagtaaactgcatgccagcttatacaaagcaacaaatgaaaaacaacacacaattacacactaaattatacagtaagtaataaacatttacacactcaacactaattgcatagctttcaattaacatttaacactatcttgtcttttcctttttcctcttcattctaacaagtggtgactataattattttaaccacaaacattttaccgactccacttaattgcatagctttcaatgaacactatcttgtaattttcctcttcactctaaatgttcttttaccataacttaaccaaacacaaggatcgtgagggaagatataataatacaccgaaccattattctaaagtcaacttgctaggtctagaatatctcttagcttcatattaagaagagaccacaatcattgcaataagcaaaccacaactcccatccccttctctcttctccacaaaca

SEQ ID NO: 122 - DNA, 3000 bp, Cajanus cajan
  source 1..3000: mol_type = genomic DNA
atccaaaccaacaaacccagccacatacaaacacaaaacacaatacataggctccatacgactcaaaataaactcaaatctcacatgcatccaacctatgcatcaaaatcatcaaaatcctcttttttctcaaaaccccaaaagatgagattttcttcaatccatctatggattctcaacattcatgcacctattgagtttctaagctcacatgcaacctacacaacaaacattctagcttcccttacctgaaggttggctcagaatagccaacaactgtgacgaaatagaatccccttccaaagctcaacccggaacttcaatcatcccaaaacaagctttctaatctgaatttcaagaaaagccagaaggcattagtggtctagaggagaaaaggagtcaaatagagaaatttgggagagaaagaagctcactgacccagagaccttctctctaaaacttctctctaaaaactcacattttcctgaaggcccctaacccctatttatagattttcggacttccccagaacggcccgtaccgagttcggaacggcccgttccgaactcctaattcacgcacaaaaaccacgcccagaacggcccgttccagattgggaacggcccgttccgggctactaaattagctattttttttaaccacgttacagatattgcacctaagctggtcgagatagagagcctcaaataagaatgacgctcgacatgatatcagaattcttcacttggagaaaccaattttaagtaacaacctaaaactcaacaaagattcaacacatgcaacactataaaccaactatatacatgacaaccaacaaaaaaatgtacttcaattcaacatgcaataactaatttattttgccatattcaaaccaacatgcttcgaaaaccccaatcccaacttaggtcattgtacttaaaatatggaactcattagtaactcgaattaacatcaaccatgacaatgcaacaactttactatactaaaaattttctagcttcccttacctcagaatcggagagaattggggaaggagaagaaggaaatcttgaacgacccacaacacaactctaacaatgaatctacaactcaattcaaggctcctaagcacctgaattcaccaacaagactacacaatgagaatcaaagaaggaggagaaaaagattgagagaaaagagatgctcatggactaaagtctttctctctagaatcctttctctagacttatgagaaaaagagtttataagactttatttaaaacaaaaatataatatactttttctcttaggatcaaccaactctttgctgaaataaactcccaacactgaaactctcaagggatcctcaaactacaaagcctctcaacattgattaactgttcaacttatcgacatgaaacaacttagaataattataacacttcacgacttataatgttacaacctttaatgaagtttctcattccaatcacttttttttcatatacaatatttaaatccaagactttgcttaataaaacctgttatacaatgcataacattaaaaaacatataaaattttgattttttttttattatagatcacaactatttttgatgagacaatcttattatggttacgaattttttttaaagtatttaatacaattacatatttattattaaaactagaactagagtcctcaaattaaacatagataaacaccatttacttaatgcatcctacctgttttcaaattagaaagagatatgcagttgcaaaattagacaatcatttgtttttattcgtattatctaattcaacatataacttttagtctgagatttcagcccacctgacttttttattttcacaataccaatcaatatatgtgaatttacttattagagagatagaaaaagatgatgttctacaatttattgtacatttatcattcctcaagctgtagtgctacactacagagatcatgacttgcaagcactaacgtacctttccccttccaaattaaattagaacataacggaagctggcataatttgtctacatataatccaaaaaatacttgtgtgcaacggcaatattctcaacaaattggattcatgttaatttttagatataataaattttaatttaccataataaattaataatcattctatatggcaacatgatatcacgtgtcagataagaatttatcgtgcaatccaacaaaagctcttcaaaggatactatttttaaacgttatacaaatcactggaaaaagaagacatcggcaaaacaacgaaaaagttcctgacatgcctcggaagtctacaattcacatgctccgccagactgtctccttcgtataagaagttttggtaataaagtatagatttaaatactttttctttataattttataaactatatttttcaattcttattaaaaatatactatttcaatttttatatatactatttttattttcttttagttctaaaataataaacgacatagactaacaaaatgatatttagagatttattaccttttgtttttaactattaaaaaactaaaaggtaatacagttattataaaaaaaacatttaaatctaaaatatataagcaatcaatcttcgatatccccgagtttttaactgccagcaataacaaagttaataaatgaaaaacaatgtataataaacatttaattagtaaacacttgattcaattgattgcaccatttagtattctcattttcactaccaaaggagtataagaaacgtgtgagaagataaatattccttcgctacctaaccatcaaatgcaaggattgtgagggaagatacaatatgaggaagcattattctaaagtcaacttgcagggccaaaaaatctcttagttcatttaagagggcacaaacattgcattaagcaaccacaccacaactgccatcccccttctcttcttcccctttggcagccgtcataaaca

SEQ ID NO: 123 - DNA, 3000 bp, Cajanus cajan
  source 1..3000: mol_type = genomic DNA
tatttcaattatccattctgttcatctacgcatttatatgtatggatcccattgcttgttaattattgaagaatctttagaaacaagtaattactaaagtctcttactactctgtgtctcgttctatatacctaaattgaaacattttcgtgctagtctaaataaaaagaatctacaaattccaccgtaaacagaaaaatatttaaaaaattgattccactttaaatttaagtacacccaagtcaacacataagtagagttgagtcatgtaattagatttagtccacttaagttaacttcatcttcaaacagagtctattgatttctctagcatagtaaatcatcccctcaaattcaaatcttaaaacttacaagtgacaaaatattattccacaatttaaattgagggcaattttattgatagaaaagtattacttaacaaatttggaaaaggatactatgtaaagttaatgatggagagagtggtattgattctattctaactattgcaaagaagaagttgtttataacttcttttcctatacaagaactaaaagagaaaaataacttcgaacaatgtcatatggtcaacgatgtccgaaatcaacaatccaaagaggttggatttttttttttaatgaattacaaagggcaacaattgaatgattaattacctttttaagcaaaagaacaagagagattagctgccattttctaagagaaaaaaattatacatgtgcttcaattattttttattttttgtgaaaggagttctcttagttgtaaatattgactcaagtatcttcaaaggtggtagacatcacttgcttctttattagatttctcttgagtattgatcagtgtggcaaaacctccaatttgaggatccatgcgatcaatcattaaatggatcatagtttcactttcgatcgttaaacgggagaatatgaattgataccaatcattataatttcacaattggagaaataaagattcaaagctctttgttactctattagaataaagaaagaaatgtttatttctaacacattttgaataatacagacgttgatttatataaacaaaactagtcctaaagtaaaatatcatgatgaattcctatatattaaatatgaatatcagggtttgaatccttccttgtgtatctaaccgtgttaaattttggggtcagctatcctaccctttgggatatctccaacgtagcgaggagattagttattgttatcggtcggcggtgaatactccaggaacgaacaaaaaaaaaacgaataattttttaaaataaaaatatttttaagttatttttctctaataaaatgatttttaaattttttttatcaaattcatttttttattataattatatataagttaaattttaatatttttaaaacaaattattaataaataaaaataatcttataccacgatataaattatttatcatgactataaaatataatttatacatccaaatataatatttattcgtatatataatttattataaattttaattatatataataaatatttaaattgtgcattatgtataataaaatactaagttatataaaataatgattaacatgtgcgcgtactaaaatgctatttaaggggaaaaaaaatcagaaagtaataaataaccctgtcacttatatatgatttgaccatagttgagattttgtttaaaattaatttttgagcacatttagaacaaaatcaaacttgaaatactcttgatctgtataattaatgtataagatcgaaaaaaagtagtattttatattatttctatttatatatcttttgaaggaagggagaatcctcgagagcaagactagtgtaaagttagtgtatatgatattaaaaatgggttttcttttatcattatctgttttaaaaaaattattatataactttgagtgcatacaattaagaaagagacaaacaagaaaaaaatgtgatttgtgatttaattaataaaaaaaggagagaaaaagttacaaaattattaactaaatatgcaaaatgtgatgcaagatacaatatgcaaggagcattattctaacaaatatgaatcacccttcgaaagtatgcaattatgaaggatgaattatttatttacataacttgggagagaaataaaatcggagtaaaagagatagaattatagatacaataagtgatacaattgaaaaataaaatgtttgattgaaggtgagacaaaaatataatttatttctaaataattgataacgtgttttctagttccttcaattttaaaaagaattgttgatttttttttatgatacactttataatttttataaagtattagttttattttttatttttttatctttatcaaagattgtgaaaatatgtatttctctcttatatattttagattgatagattgtcatttattatttttaattagtattgatatgtaagtttttatatgaaatgatatttttatctctctaataaaaaatttaataataattaaaagttaaaattgataattatcattttattttataaactccataaaattaacataattataattatttatattgattcattgaatcgattaaaaatatttcataatatgtagatgaaagagtataatttttttaatatattttttattattgactaaaaatcattgcattttttttctctttctttctcttccttccatccataagtggatattagaagaaaaaaaatttcccataaaagatcattgaataagaaagagtaccaacaatttgctttatttttaatgaacttcatttaatacaaaagtctaaataggggcatgattataaatttacagcatttctatattggttgaaaagtcaacctttcatgtgcaaacaatctcttatctattattatataagagtcctaaggcagcaatacacaatattgcaatcacaactccaatcctattttcttcctcaaaaa

SEQ ID NO: 124 - DNA, 3233 bp, Phaseolus lunatus
  source 1..3233: mol_type = genomic DNA
tattggaccctaatggcccattctctaaaagtccatttgttttaacttaagcccattgttaattactaactaaaccttctctaataattaaaatacttattattaaatctaatcttgggatgttacagcgtgactgacaaaaaaaaaaacatatagggagaggagcatgtcaggaagtttcccatatgcatcaatcataatatcacagataaaatatcctcaaacttttctaactttcttctcaataattggtctgcaacattcaactctgtatttctaatgaaagtttttcatcatgcacacagtttccgtgaattgaaaatgtactacttcactcacctaaataaaatagttctttttcatgtgaactctaaggtaacaacaggttcttttttttatcattttttttctccttcacagttctggattcataataatgctatattatttattgtcaaatctgatgaatctgcacatgctaatctttttagtgcacaaaaatgttatacattcaaagaacatatgggtgaagatatagcattggagcatagataccataagaaaattattgaactcactcaccaaacacatggccccaatatttgtagcatagtagcttcttgtaggtattatacattaactattattttaggcttttgggtcatacatggctgcccaaattattcataaacttgacttgtgctccacttccaggatcttgacagcgtatacaaaatttcataatatgtatactaattgagttatgatatcttcagaatataatagtgatagcctgttatattttgaaatgtattagcatattaaaaggatttaatactgttttttttaaagattaatttctacattttttaaaagactagacaacaattacgttaaattaatgaattaaaacatatttcataacttatgattatttttcaatatttttttttctgcattagctttatagatactctttttatatataaaaactaactttatcatattgaaataaattaattatcttgtaaattttcttacttaagataaaacaagttctgattataattagttataataattcatttttacagcgtaaaaataacatgtgttctaattgtaaaagcattagtatgtcgaaatgctttaaaattattgaaataaaaaataattaacacaattgtttatatctaaaacaaaattaccgaaatgttgtggaagaaaggtaatgctgtagaaaaaatataggcaatgaaaattactgaaacaaaataacttttactaatataccaaacaaacctaaagaaagaaaaagaacattctttgcaattgcaaagcaaagacttcgttgcctttattgaaacgtcagtattgactaaaactgaaaatcaaacatatatcaaagggaattgcatggcatttaagaaaaactatttttaataataatttttttattaaaaaaacattgctaattcttttttgaaaaaaaataataattgttttgctttaaactatttaataaaccctaattgttttgtacaattaattagttaatcaattcagggattttgtttgataaataataattaagaaagtccaacggtggttttttgtttttgtagtttgctttagaatatgtgaaagcacttttgtctgtaagttgaacacatttttgtttgttgtttaacggtcacctgctacgtcttctataagggagcgttgtaacgtaaaccctcgatatttgatttcgtacggattacgatttttatttttaataatattcgatatctcttttaatttaatatttcaaaaaagaaattttcgacattccttctgattttttcttaatttcttaaccattgttttaagactctactcaacccgattcataatattatattataatttaacaattaatgaagagttgaagattattgaaataatttacactatattagttcaacggtcatctatatcatgctgcatcttacttacaacaactacttatttatttctctacacaattttaatttaattttttcttaaataattgtttatagattttcaaaagaagaatacatttaaatttattgcgttctttcacccctcgatatatcctcgatctaaaatatacataatttttgcaatttctaaacactaaaaataaaataaaaactgaaatagcgacattaaatctacaattccagaaaaaaaaaaaaacttaaccctagctaatgaacataattttgataagaacaatttaagatgatcagataaaagtttacaatgattagaagaagaaaaagtaattcaataacacttttagctgatatctaaatagttcattatgagtaaccaattatgtaatgtattatactttagaataatagttagataaaaacgagtcattctaaatcctctaattatttattcctgctacttttggtatagtagataaataaatatattgaaccattttatgttttcaagatgttaaccatattttatatttattattggttataaacattaagatgatttatacaagaatataacattattcataataagagcttatagtgcgatccattaagaaaaactcttaaaatatgccatattttgaatattaaacaatacattgaaaactggagagagagaaagaggtttaacaaagcgaagaaaaaaagtttcccagaagcctgaaagccatccatccataatccatatactccaaccaaaccgcagccaaacacaaaccaacaaatgaaaaaaatatacaactattataatttatgaattaaatatcttgtttacacttcatagaattattaactttgcttctcatatatatatatatatatttattaactttgcttctcatatatatatatatatttatttttgttcaacattaaaatgagaattttttgtattgacagtcatatacaaattctgcaattttataagagagaaaaggctatttaagattaaattataaatgtataatttacctactcgacactcaattacataggtttcaagtgtgagaagataaatgttgttttagcattgcttaaccaaacacaaggattgtgatgtaataatataagaaaagcattattctaaagtcaagttgggaggtgaaagaagtctctgagtttggttcatataagagacagcaagcattgcaggaagcaatcacaattcccctccccttctcttcctcacaaaca

SEQ ID NO: 125 - DNA, 3000 bp, Phaseolus lunatus
  source 1..3000: mol_type = genomic DNA
tacataattattatatttctattaaagacacccttctaatttgatccaatttgacctggattgataacgtaattacacaaattttatgatcatataagatggtcccattatcgtaaatgttttgttcagcccaattcattagcccacttaagacctccacattatcgtaaattgcatgttatttatcttttcctttaccgtctattccatccaattatctttagtatcataccattgtgacttagcttccacacctatcttgcttgtcataaatgaatcttcaaactccattgtttcatacctatggtctatctttatatttgcatacaatttggtcatcgtcctccatgatgataactccattaaagatttttttctatgagaaacttgatatgggattgatgtcctaatgaatactccctcaactctaatttttttaggaatttattgtgtttttctgtataaaagaatgtacatcgtaatgtttaccaatttttttatgcattcaacactcttgcacaaaggaatattgtaagactatacacatactaatttattacctaataacaattatgagtttcacttctaaatattattgaacatgtccccaacccctttactaaacaacatgtcatttatacaattttcttgtgttccaaaaatcaaagttggattgtccttaaaataagacaatataattcctctcattttccataaatctcctccaatactaggtagattagtttattaaacattgttaggtcattgagccttctcgtctagctttttgtaagccatttttcttctcccatgtctgaatttattatcatatcattacatttctcaacatggtcttgaagtctcatcctagacttccatttttgtgtagtagcttttgacttcaccacttccacatatgcaagtttgtcagtcatgtattgtcttccatgttctattatactttgttttttcccttattttgatgcatgtgaagtttttgtctttcaatttgtattatttgtacccttttatccttaaggatgttaaaaaacatttcatattcccaatcatgacatgattaagttctttttccatttgtctttcatctctcacatccttaaaccttaaaaattcaaatctttgtccaactctattccttcacctagatatgaatatctctcaaactcacctcatctcaaaaatatctttcacaaatcttcttcattgtaggcgcctaggaacctataaaagaagaaggtagagatgtttttccaaactcaccttatctcaagaatattttctatagatcgttttcattctaagcacttgagaacttatatataaaacaagaaggtaaagatgttgtttcaataattctttcaacttttcttttcactggacctcgcttacgatcggttgctccccatatcttttttttatctaatttataatcaatgtgaaatattaaattatttttagtatatcccctcatatctataatattagtgggtttaccacatgaataacataataagtggtttaaaaatatatttaaatattttcttaaaatttgagattgagttagtttaactccatactaataatctgtactaataatctgtaaagtgtttaatattctatatctagttatcttcatgagaatttgtatttaagatctcttgccaactaaaaataatatcaatatttataattttcatcttataaatttattttgtgaaatttaactaaattcaaatttatgtttttaagataatgttaaaatcttttctatacattgtttattgttgtgtcaaattacatacaaatagttaattttgcaatctttatgctcaataaatcttatcctgaatttaagaaggtgttacatgtccaacttagacggctatataacaaatatattacttacaagataataattatctaataaattaatggattgaatgagatttaaaattgatctctaagtttttttttttttgtcttactaattatttaaaacctctttattgattcatcaacaatctactaaaaaatgcagtctttgtaaacttgacaaatttttaatcgtgtaaaaaaatgtgataatatcatatgctgaatgtcaaacttgacctatactttagatatgattatcatacatctagttgttctttcatcttctttattatatctattaattgtttttctctctttatcttgcttgcttccacttatagactctaaaatgacattcagcattcagcatctttccgtaacgattaaaagaataagaaataataagatcaacacattattttatttttagtgaaactattttaataataaacactgcacagtggcagccgggcaagattataaatttgggaagtcaagatttattatcccttcttacaactacttttattaatctctttaattaatatttcaattaaataacttaattcaatattttattagtatatagaattattaaatggttgtaaatgcatgcttgaaaatagttttagttttttctctaagggaaaaattatttcaacaccattttttataaatatatccatatcaaaactgtgattaaaataataaaatattaaattaatatatttaactcatgatacgtttacaccactctttctttcttaacgttcatcttataattgtatatattattattattttattgtaatatagtattttattagaaaaatcagttaaaagtaatgtatttaatgaataagatatttttccttaactaagacataaattaaattttaataaattattattattaagagttgttaggaagtggtagaagtttataaagaagttttaagtatcactcttcttttcttaattataccttagaatttctcgtttataaagtcaaccaataggccaaacaaactctcatctctttgcttataaagtggcttaggccgcggcaccacacaacactgcaagaagcattcacaacctaca

SEQ ID NO: 126 - DNA, 3101 bp, Phaseolus lunatus
  source 1..3101: mol_type = genomic DNA
ggaatgattgtcccaccatttcctaccctaataaagaatataagagcaagggatacccctccaaatcaagaaatgaaaggtcacgagaaaggaataggaaaaaagaaaaagaaaaagcaaaaagaaaaaagaaaaggaaaattcaaaactagtgaactatataaagaagagatatttagtgctttaattgtcttggtagaaggtactatgatgttaaatgtctcaatagaagaacagtgtttttaaaagaccaaaagattgaaagtcaggatgaagctcaatcttcacctagtgaagatgaaactctagattctgatgaataggaagctataccttgtgaatgagacttgtaaatggtgagaagacttattgaaagcttatccatagaacttgaaccattttaaagaggaaacatattccacaccaaatgtaaagtctttgtaaaaacttgttatttgattatggataatggttttttgctgtaattattgtagttctagattggtagacgagctagccttaaccgctacatcccatccaaaaccttacaaacttcaacggataaagatgatggtggtgtagtagttaattaacaagtgagtatcttaattatataagtttaatgtgacatagtctctatggaagcttggcatattctaattggtaaaccatgaaaatttgataaacaatctatccataatgttcttgtcaataaaataagtttctctcacaagggcaaaaggatagttgtgtatcctcccacaccttaacaagtgagagaggaccaaataaaaatgaaaaaaaaaataacttgaagaggagaaaagatagagtaaataactaagtcagaaaatctctctcacacttaaaatagatggtgagaaggaattgagtttggaggtttgtgtccctccaaagaagttgttaaacaaaattttttgaaaaataaaataataagtctcttctagttgaacaacctatttttcttttctattgcaaagagatacttgttaccacaagtcgtgaacttgattctctaccacatatatagggttcaacaaccttaaacaatttggtgattttttctcaagtaggttcctcatggatttctacctttaagggaatataatatcaaatagacttagtttttggagtagactacccaataggccaacttataggactaatccataagaaactaaagagatggaaaatcaagtaagtgacttgttaaaaaaggattgagtaaaaagggtctgaatccttgtgttgtatcaatatttttggtccttaagaaggatgggtaaaggagattgtaggatcattaatcacatcactataaaatacatgtaccaattgctaggttagatgacatgaaattaacatattttccaaaattaatcttaaaagtagttattatcaaataaaaataaaaggaaatgaattgaaaactatttttaagatctagtttgaattgtacaaatggttagtcatgcattttaatttaaccaatgttcctataatctttatgagattaatgaattatcatgtaattagcgatttcataaaaatatttgtggtactctactttaatgacctcttaatctatagtaatttaaataagaactaaaataaatcttgttgactccatgaattttgaattcttctactcaacttaagcaatattttagaactgcaataaacgtaagtagaactaacttactactaatttctcttaattactaatctattttaaaataaatatataaaataagttataaaaaagagttttagaagattggtatagttttacacccatgttcaaaatagaaaagagaattccttcctacaccccattaattctaacttgcacccaatgaaaatttgggatagattttgtgttatgaaaatgaaattttgaaatagaattaaaaaaactaaaatatcattatgaaaaatcaaatccaaaatacaagtaatgcacgaaagaaatttttaaaagaaaacaaaatctaaaaaatattttaaaaaacaaaaatatgaaaacatatcttgaaaaaactattctggaaagttaaattcaacaaagtattctaaaaactcaaatctaataaaagcctataatacataaactcaaatctagaaagtaacaattaaggcaatttaattacatagactttgggaacacaaaatctgaagttgtttgaagcctacaattgaagtctctttccctacatcataaatcatagtaattccttaaccgcgtaaagttccttagattgtgtctttccttcacactaggacaagacatgccccataaattcaaactgccatatcatgacgccatcccttccttgcactttcctaatcagaaaccttctcacttcctttcttcacacaatctaattctctaaccttcccaacccaacaacaacataccctaattccttcaccattcaaccttcataaaccccctcttacccatctcattcaatcatatccattccaatccaattcctctcttctctatcccatgcaaaatcataacccaataaaaccattctccttgtcaaaccccttcaaccactccacccaatttagtgtcacaataatcacactttcttgcttaaccaattctccttactctcaatacctgaattttccctaggatcaaggttcatacttatcataccaaggttacagtttttttttttttctctctagagcttgtctttcttaaaaaatatatttactttttaatataggaatttgtagtttatgattactttttttaaaaaagaattcttttttagcctcatttgttcacgtgtaaaagttgttttttttttcatttaatgaaatttttttatctaaaaatcacatttttcagaattttttctaaaagaaattcacatataatgaaaaataatctaactttcacacaaacgcgtacttatatcaactttaaaaattaaaataagcttattaacattgtccttattagaattttgcaagtagtcttctaataattaatttttctttaatactaaaatattaagaaaataatattgcaaacatagatttagtcaacaacctagaaa

SEQ ID NO: 127 - DNA, 3000 bp, Phaseolus lunatus
  source 1..3000: mol_type = genomic DNA
tatatatatatatatatatatatattatttgtattttttttaatatattttatttatcttcatagtatttcctgagttaattattaaagtaagagtttagtaaataggaacttcagttgctatattgataaattttaaaaaataatttaagaatatttgtttagcgataaaatttataaattataaattgaataattcaaactcaatttgaattacttatgattaaataaatgagtaagttggagtttttaatttttaacagttttttttatcaaataataactgttttttattaattaaaacataattattaaaaacatttctaagtctccaaattatgtttaaatccaatcaacaccaaataaaaaatataaaagtttaaatctaaataaaaaggaagtgtattgaatattatattacattttgaaattttattctttatttaccatacaacaaacaccataagtactaaataaataacatttaaatatattaaatgttttaacttaaattgacttaaataaattttaagactttgatatttattttgaatacttaatatttgttcaaattaggtatgtaatgaattaaacattttagttttattttattttatttcttatatcaattctacattcacatattttttaattaaatattatgtattaagttatttttgctatatgtataaataatacacattatcataagataaaaatgtttagattgttatgataaaaataaataaaaaatcaaaagagaaatttaaatatattttagagattaattaaaacatataacatattaagaacaaattataaataaaattttatttatagagttaaaataatattttataatttattaagtacttaatttagaaaaataatttgttgggtattttaataaatataatatatataatatattagggactcaatttatataagtaatttaaattttaaaaaaattatatgattgacctgatttgcaggcacggttttagcaggaacttaaaagcaaatttttagaaacaacagcgtttttttcccccttctttctactgatttctttcggcccttgtccaccttcatcatggctttccatgaatctacacaaattaggttgtttctttctgcatctttataattttgtgacactctatgctttttaaaatataaaaaataatttttattttattgatatttttcatttcaaaataaatgattcagataattctggattataaaatttgattccagattctgtaatgtagaatgagtttctttaatacaatacatttcaaattatgaaatgtagaagatattttcgtattataaaatttgagaagtatttttaaatttgagaatactttttggattttataatctgaaatgcatttttttttaaaaaaatattataaattataaaatctgaaagatatttataaatttaaaatacattttagattttacaatgcgaaaagtgtaaatatgaaaaaatacattttacagataatagaatctaaaatatatattttttttttaaaaaatagtcttttcgctaacctatgcagtgttagaagaagttatggaagtgtagaaagaaacaacccacaaagtaattgaaaaatccaaatggcttaagcttaaaattttgagatatttcaaaatttgaaaacatattttagaatggagattctgaaatatatttttgaatccaaaaatatattatgaattttcatattaagttattcaaaatatatttttaaatctaaaaagaattatgaatttttaaattatataatttaaaatgcatttttatattcaataatatgaaaatatatctaaagtataatttagaatatatttttatattatataatttttaataatattttttttattgtacaaagtaagatgtattttttattaatgaaatctcgaaatatattgatattgtataatctaaaatgtatttttagagtctacaatccaaaatatattttatattgtataatttgaaaaatatttaagattataaaatttaaaatacattttcatataaaaaaaattattatgatttattatattctagattaaataatttagaatatttttttaattatgtaatcaagaatcattttaattatacattactcaatttagaataaatttttacattatgtaaaatataagagtttaccaatctataccatttgaaatgaaattttagattaaacaacttagaataatgactcataaataatttgaacttttctcacaatagctaaaaatatatggaatgaagaaagaagttcccttgcatcaataggtttataagacaataaagtgttggtaggtgttagagatattttttttaaaggggtgtgatttcattgtagaaagcaatacataaattgtatcaatcatcatagtgtgaagtgatggaactctggtttttaatttacctctaggtgaagtaatgcatggataatttagttgcataggaaaataaatctctcaaactctatttagaattactgttcttaaaaattgtgagaaagtaatatgattcagtttgattttttaaaattttagtattttgactctaaatcataattgtattgtgaatgatatacaagataatctactactaaaagtagcttctccataaataaaaaaaaattaaggagaaaaaaaaattctttgaacaagttaaagaaagaaaaataattctaacactctaataataaaaaacctttgatccttaccccattttctgcactcaagccaaagaatggaatcatttcttccatttttctgtttctatgaacattcttccattctcttcaaacaatccctttcttcataaatttccaaattccttgcttttcttttcatcttcatttttctcttctacattaagtacagtaaccaaccctttctatttccatatccaactaacttaacgtcttccaaatctaaatccaaactcacc

SEQ ID NO: 128 - DNA, 3052 bp, Phaseolus lunatus
  source 1..3052: mol_type = genomic DNA
gatcctttctagtatggatgatcaggttatgcccaagtagatgcaagggtccctaattatggtttataaattgtttattgttcaacacttataaattatgttgttagtctcttaatcctggtaaagagacctttattttgtatgttttcgaggtgtcaaaatacaaggtaatatctcgacatctcagatactccaataatacaactatgttacaaataaagaaatatcataattttgtatttattataattacaaataaagctaaaaaaaagcatgaataataaacaagttaacttttaaaattgagaatattgcacattagaattatatttagtaaattcaaatataaactaaaatatctataacaaaaaattaaaccaatgaaccataaactaaattttctattataaagaataggaaattcaactagtaagagttgttacaaaattttaatatttttagtttcacattttttctcgtgacctattttaaataattatagtaatgatgatttgattgttctaatatttttaatcgtcttacattgtttagaagttgagaacaaatataatttaaatacattttccttccttaactctctaaggtgctttttaaggataaaattgtgatagaacttcgagtttcaaaatagacaatacctcagacacaattattgaccctaacaaagttacctcaacagacctgaggttggaactcttattttccttagacgttgatgagagacttgttgtgatatgtctgatagtcctatattgcttagaagtcaagttataaataatttaaatatatcttcattccttaacattttaaaacgccttttaagataataaaattatgatgaaacttcaagttctaaaatgcacaatatcacaaacaaatatgatgattgattctaataattttatttcaataaacttttatacaactcttaccagtcaagctcctttctaataacaacaataatgtattattttttctttcatatgtagttagtcctcggagaggatttaaaaaattttaaaataataataatggtaacaataataataattaattgttttgtaataactaatcaattatcttataaaataatcaattgggataagtaaagtaattcattatttgaagttagaaaaaaagtacaatatttgtggagaatatattacgattaaaagtaataaactatatcatttttattaaatttgtatttttttttattaaattaataaataaaactaatcaatttacgaatcattcttgcttatttttaattgttcttcaataactctggaagattaaacgtaatttttgctaaaaaatagtaaatgatatattgcataactctttatttttatctttttggatattacatcaaatgatatatttcatatactttgtgaattttacaatcattttttaggtggttcatgtaataggcatttacatgttgatctaaaaaattaaaatactttaaataaatattgtacttgttgtgttattttacattaattaatacttttaactttttaatttaaacatttctttttcaaatatatgttacttatcttctttacttattcattgttaataattatacatatgtttatttcattatattaaatatttttttatttctattcaaaattctagataaattaaaaaattactcttaaatattaaatttatcaccctaaatataactcgacaatgaaaatttccttaattgaaaattcaaattgaaatacatttacaatttgaagggtccattatcaaaatagttgtttgtgtggaaaagtgtgttgtggagagcacatccttcctccatgcttctattttggatgttgacttcttcctctctttcataccaaaaaagggtaagacacacgaaatagaaaaaaaaaataattaattttagagacataaatggaacttaaaaatattagaaaaattttaagttttctctaattttagaaattaattttacaacaaaatacaattgtgtatatgtttacaaaatgggaggtattcttaatttttttatctagtccaatccaacttatttatatttttatgggttggattgaattgttgtgtgtcccaataaaattggattccctaacatgtttataaatttttaatacgaaaaactcaacttcatagaggatactaggtattatttgtgtattttttaaaattactcaattcattatcaatccgattataatacgttagatcggtactcaataaatacataaataaataaattggatgggatgcaattaaatttaattaaataaaaatcaaattaaactcacatctcaacgtgttgagtagtatatatcatagacgagaaggaaaattcatacatgccaagacttttccaaacaataatatattttggcttgctataaaaacttccacatgcaatatgatccatatgtgtattgatccataacttgtgtttatcttgctcaaattctccatttgtgtgcacataaatgtgcttttatgtggggtactggtttttttatttttgcatctgaatttggtatgttcccattgagaaacttctgttttgcttcttgacatggtcacattcactcacctttctgcccaagtttcacttcctttcaatcaatcattcaatcctctttactcacacccttccacaattcattcaccagaaatttagaatcattcatactctctaacatatcatcatcaacacaccaaaacaaaaatttaacctcaacttgtactaaaagccagttcaccaaatcaataaactatggtcaactatccagcaagtatatcctccaaaaatccaaaatccaaacacctctttcctactcataacaatttaataattaaccagttgttgatagagtagccgccaataatgaaaaaacaccaagtctaagtatcaagtgcattcacctcatcctctccaaaacccatatatacatcacaccattcaagcattcatcatcaccacaaccaaacaccacttccaccaccttcagaagcagca

SEQ ID NO: 129 - DNA, 3251 bp, Phaseolus acutifolius
  source 1..3251: mol_type = genomic DNA
ttgttttaagttaagcccattgttaattactatctaaaccttctctaataattaaaatagttactaaatctaatattgggatgttacagcgtgactatgaccaaaaaagcatatgaggaagtttcccatatgcatcaatcataatatcacagatacaatattctcaaacttttctaactttcttctcaataattggtctgcaacattcaactgtgttctaatgaaagtttttcatcatcatgcacacagtttccgtgaattgaaaatgtactactccactcacctaaataaaatagttctttttcatgtgaactctaaggtaacaacaagttctttttttatcatttttttctccttcacagttctggattcataataatgctatattatttattgtcaaatctgatgaatctgcacatgctaatctttttagttcacaaaaatgtcatacatgaactattatttttggcttttgggtcagacatggctgctcaaattattcacaagcttgacttatgctccaattccaggatctggacagcttatacacaatttcataatttaatactgttttttttaaagattaatttcttcatttttaaaagactagacaacaattacattaaattaatgaattaaaacatatttcaataacttgtgattatttttcaataacttttttctgcattagctttatagatactccttttatatataaaaactaactttttcatattgaaataaattaattatcttgtaaatttttttactgaagataaaacgagtattaactgattataattagttataataattcatttttacagcgtaatattaacatatactatatatatatagtgttctaattgtcaagcgttagtatgtcgaaatgctttaaaattattaaaataaaaaataattgacacaattgtttatatgtaaaaaaaaaaatactgaaatgttgtggaagaagggtaatgctgtagaaaaaaatatacacaataaattactgaaacaaaataacttttactaatataccaaacaaacctaaagaaagaaaagaacactctttgcaattgcaaaacacgtctactgaaaaagcaagactttgttgcttttatgaaacgtcagtattgactaaaactgaaaatcaaacatatataaaaggaattacacggaatttaagaaaaactattttttttttaaatttaaaataatagtttatttattaaaaaaacattgtttattattttttaaagtgtttcttttggaaaaaaaatataattgttttgctttaaactatttcttaaccctaattgttttgtacaattaattagttaatcaattcagggattttgtttgataaataataataaagaaagttcaacggtggttttttgtttttgtagtttgctttagaatatgtaaaagcacttttgtctgtgagttgaacacatttttgtttgttgtttaacggtcacctgctacgtcttctataagggagcgttgtaacgtaaaccatcgatattgatttcgtacggattacgatttttatttttaataatattcgatatctcttttaatttaatatttcaaaaaataaattttcgacattccttctgattttttcttaatttcttaaccagtgttttaagacactactcaaccccattcataataatttaacaattaatgaagagttgaagattattggaataatttacattatattagttcaacggtcatatacatcatgctgcatcttacttacaacaactacttatttatttctctctacacaattttaatttgatttttacttaaataattgtttatagattttcaaagaagaatacatttaaatatatttttgctacccttgaattttttttattttcgtctagaatttattttctagtcctcacattcaatacaattattgagttttttttatccttcaatttatcctcaagtcgatcaaaatatacataatttttgcaatttctaagcactagaaataaaataaagactgaaatagcaacattaaatctacaaccacagaaaaaaatacttaagaacaatttatgatgatcagataaaagtttacaatgattagaaaaaaataataataattcaatcacacttttagctgatatctgaatggttaatgattagtaacaatgatgtcatgttttatactttagaataatagttagataagaatgggtctttctaaaccctctaacaagttcttcctgctacttttttttaattggtatagtagataaataaatatattgaaccattttatgttttcaagatgttaaatatttttttatttaatattgtctataaatattaagatgatttatacaagaatataacattattcacaataagatcagcttatcgtgcgatccattaagaaaaactaaaaaaatataccatattttgaatattaaacaatgcattgaaaacgagagagagaaagaggtttaacaaagcgaagaaaaaaagtttcccaatttaaataagcttggaatccatcatccataattttccaaccaaaccgcagccaaacacaaaccaacaaatgaaaaaaacacacaactattataatttatagaattaaataacttgtttacacttcatagaattattaactttgcttctcatttttaatatttttaactgaagatttttctattaagtagcatcacaatataattaattaacatgctcattttttttttcttttggttcaataaattttacattttatttttggtcattttgcgttaagtaacacattaattaattttttttcttttaaacaagataaccgaaaatccatcacttaaaaaatgttaacaaataatagaatccaaaatacaataaaaatgattgaaataaaaaacaagatgtaaaaaattactgggaataaaagcaatatatatattaacacatcacttaattattaatatacaaattctgcaattttataagagagaaaaagctatttaagcttaaattataaatgtataatttaccaactcgatacttaattacatagctttcaagtgtgagaagataaatgtggttttaccattgcttaaccaaacacaaggattaaaaagcattattctaaagtcaagttgggaggtcaaagaagctcatagtttggttcatataagagacaagaagcattgcaggaagcaatatcacaattccccttctcttcctctcaaaca

SEQ ID NO: 130 - DNA, 3011 bp, Phaseolus acutifolius
  source 1..3011: mol_type = genomic DNA
taccaaatttagaatatgttttttacaaaaagacacaaaatgttttatgaatatatcattttcgccaagacattaaagagcatatcgtggctactactttaagtagcagtcccaccttcagcacattctcaccgatgggtttaaagtcttgtagttaactatctttaacttgtgcttagcttgcttagttagttacttgctttgtttgttaattgctttgcatcgtttttcttgcataaaatttgacttttcattcctcattatggtataatttacttagaaaaagaattgtgtgaagtttttgatggatagttttggctaaggaaagacttggtacttaagtcctagtgactcacctcttttcctggaaagctaccttcacaactttcctcttctttaataaaatacttttaatcacaaatttttagtcatgtcaacccatccctctaaatctaatgaaagtgatatgaagttcatcaaatctcttttaaaacaacttgccaaggatttattggtggacggctttagaaagagagaggcgtcttcataaggagcctcccatagagtattggaatgaccttaggggagcctaagacatcgccatattccctcctactataatagggagttgatggataagctccaaagactccatcaaagaaccatgagtgtagaggagtataggcaaaagatggagctttacatgatgagagcctccattagggagaatgagtccaatcctgagataagggatagggagaatgatttggttcaactttgcatcaattaaagttgaacaacaaaatttaaggaaaacttcaagtcttgatatctaactcctattccaagagagattttaaaaggaggagagtacgtataagtaaacaccgaaagagactctaaacccttaggaagatatgctcactccacccatttaaatgttttggaagaggacatctgcaagctcaatgtcccaaccaaagaaccttgtttttaaaaggaatagatgaatatactagtggtgatgacaaacctagtgagaaagaaaagatgagagatgaagaaagagtgtatcctttagagggaaattattaaagtaattgaatataattgcgtacaatatcttgttttgtaacttccactttttatttatgttgttttagctatgtctatatccaaatttctaataataagattctatcattattaaattaattgcatataattgcgtacaataacatacaataattgcatacaataagtacatataatttaataacagagtgttttaaaaatatattattgtcatctaatatacctattacatacatcctatttgtttctgaaaaataaaataaacttttgtaactaaaatgtaataaaaaaacaaaactgtgtctagtaatatttgttttaactaccaaatttataatgtttttttttttacaaaaacataagatgttttaagaagatgtcatatcaagacattaaagagcatatcgtgactattacgttaagtaatagtttcaccttaagcacattctcacttatgagtttacgcttttctgctcacttatgagtttacgcttttctgcgatttcgatctccttcaaatctacaaaggatttcacctagacatcatacaaataaatcaactttcacttaaataaactttcgtaatttctcatttttgtttatgttgctttaactatactcactctcacaatagtttattgattaaattcacttaacaattactttagtgattatctaactcaatatatttaatatcttgaaatgatatcgtttatgtaactttatgtaatctcgaatattttgattttgagctgtacaatttcttttaaaaaaagattttaaatcattttctcttcaattatcttgtgtttttgttaaaaacataaataattataaatttcatgtcacaactattttattcttcttataaattttagaaattttgttaagtttgattttaatttctataaaaatcaaagtttgtttttatttcttataatgtccatgttattttaattatagtctctttaaattatttttatttcaatttaatgttttccgttttaacattaaacagtgtacgcattttcatactatttaataatctattcctttcctttttattaatctattaatttatttatttgtttattaattttattttataatttatgaattaatttatttagtaattcatttaattgatgaatgcacaatactaatttatgtttaaaaacttaagcttctttataagaaaaaaagtatgtatagaaatttggtggaaggacccgaattttaaatttctttgaaaaacaaatttttgtgtggatttagatggtaaaatggactttggcccgctaacctgccaaattttgatatgctgtttttaaccttctaacataacttgtctcctctaacttagctcattttttaaattaaaaaaaaaaaaaaaaaatttatactatattatttagagaatgcaaatttataataacaatataacttaaatcataaacacttctataaattaattttagttattaaatataatagattactttaacattaaagtaatgattattttaatttatcctcaattaaagtttttcaaatatttatataattaaatattttttaatatgtatatataacaaaatttaaaaatacaaaaactatctcactattaatctcatttattaatctgttcctttcctttttgttgattttgtatgtctatatatatatatatatataaagaattttctttgatctcctctatacaccaccatttgtttcttttttataagtgcaatttagtaataaacatggcaaattacattttctatttatatatatagcaatatttattgagattctaaattcaatcttcatgttctaaacaatctcttgtatattaaggaggtaacactgaacattgcataaggatcaatcattcccttgctcttccatacaca

SEQ ID NO: 131 - DNA, 7243 bp, Phaseolus acutifolius
  source 1..7243: mol_type = genomic DNA
gaactcatgatacacataagtttgaacaataaaatgtttcaattcaataattgaggttgcagcaacatatgcatggaaccttgaatctcatatcagttatgttactttgttattatggatatttaaatttgttctggaaaataaaatattttatatatagactttgctccttattacagtgaagttcaactcttgtttaagatttttagtatggtctataacatcgatttcagccacaataacatctttagattattattactctatcacaaaatagaaaagagatcatgttgaagataaaaaaaataaaagcatttacttgattttatcatttatatgaaggcatgtttaaggtggtctcaaatgcatcaatctttagagaactttaaaagactatttgaagtcttttttaaggtattataacaatgttgtgtatagaaaatcttgaaaagtttgaattcttgtgtatgaaaaagcttgaagcagtttcgaattttaacacgaaggtataagattttttcgaacatggcatgtgatggggataagatgataaaggactgtgtatattgaagggcagtttttgatttatttttttttctatatttgataaatcatctttatagacattattaagaaattatttctcgagttaggaaagatggtcaaataaatccattttcataattataattctcgaacaattaacgttattataagtaaagttaagtcacaattcgttttcgtcacaaatatatcaatattttatttttttaatacatatataacttttacttataatagcaggtcctcttcataaaatatttcaataatatattttactttttaaatcacatgattaattcatttattaaatttttttctaattacttcttgcaaaaatattttttgtgaaaaaaatcttaatgtatgtttgtttctctttgtataaatttccaatcatcaaattgaataaagttcaaatatgcacttaattgtttttgggtccgaaaaagagaaacaaaaggctttcataattgaataaagttcaaatatgcacttacttgttttttattccgaaaaagagaaacaaaaggctttcattgtactaataaatatttttatatgatttcctaagccaacttatatgttatataataaaagatgtttttcaaaatatatatgcacctaaaccctaaatactatttacacttaaatattttattattaaaataaaaatatatataaaaaaatattcagaatttatgttaaaagaataattgttctctagatttttttcttttgtctttttttaactttttgaatttccgtcatcgactattaaaatttgttattagatttttttttcttctaacttttttaatttctctgtcatcaacgattattaatgattattgaacttctgacaataaatactacaagattgtctaattaaaatcttataactagaataagtttaactttgctcattcttctttaaataatttttagtttttagattttagttttttttagatattttgcatgtaatgtttcaagacaattacataatctattatctttataaaatgtttaaaaaatatctccaataaaaatatctaaaagaccttcttcaaccatattgtcctttttataaaatatatgaaccaatactattccacattgaatccgataactaaagctgaattgatttggtatggtaacaatatatatccattgagatcacaattgtagatctagattttacaaaattttaacattagaatatactaacataacataaaatataaaatcttgaattataaaaataaaatataatttttttatcttaaaaacatatttttaataatagaatgcaaattattaaaataaaatcctaatattgaagtcaacataacatatatgttctcaagagattaggtataacttaaattcgattatacaataatttaaaacataacatatgtgttatagagtgttatgttttagtaaagtagagttagatgataatttatggctcaatgaattaacagaagttattagaacatggcacatgtaaacattaatcggaggtatttaaaaaagaaaaaaaagaaagacattaatgaagaatgcaatgaaaaagttttggttccatcgttggaatttcacatgccttgaaaatgaacacatgctctgcctcgtaagggtaggaagcttatgctataaaatataatcaatcatttagaccctccaacttactctacaactaaatccaaaattaataaacaaaggtaaacctcgattattattaataagaaagaatatttacttttaaatatctattttatttatcttatttcgtttaatgtatgtttagagtctcggtcaatatcattaaacaaatatccttaattttcataaactcaataaatgttacttaacacaaaattattaaaataattaggtataaaatgttatctaaaatctgaaattaacttgataatttatcttaatttcagtccaatgttagagattcataactttctataaatgtacacatcaaccaagataaataaacacgttaaataatatttattattttaatctatctcaaatcaagttttaacttccttatcagaatacattttacaaatacccgtcatatcctgatcattagtcaggaccaactataacacaagaagataaaagaataacatttgacgtccaccattattacttttgaatatatattttttatttatcttacttcatttaatgatcttaaaaaattgactttaattaatctttgaactttggttacactatcaaataatgcttaattttaagagaatgatatttaagaaatattaaaagattaataaaattaatgtagtatgtaaaattagttttttttatgcgtataaatgagaatggaagtactacttcgaaagtagtattattttttgttttctgtgaatcatttatttattatttaaaactttccaaaatagcttattaattttaattatatgcatgctaaaaaataaattaatgtaataaacgagagatatgaatgaactataacaagttataaccattttaatataaagaaagatatcaattgtttttttaatgtacgtgttacaatttcttagaatttgagtttccaattactccagtcttaaaaaaaactggtatatatatatatatatatatatatatatatattatactcaaaacataaaaataactaataatcaaaacaatcattttcaaatgtctacaactttcacctatacttacgctaacttataaagatctattgtgtataatctacaacaatataacatacttcacaaacattatcttatgaaacataattatgaggaagagtcacattaaataaaaggtgaacaatgacatggttccattatatatgctaatgaagaccaactaattaacatattgattgacattatctttataaaactcttcaccttaaaaaacgcatatgttgtattattttcacaaggtccatgtgacaacatataatagaagatcttttatttattttcttaaacttttttttagagaattgttgacacaaatgtatttttggatcacacggtgtagaatactataagcattaattcacctataatatgatttcctaatatcataagtttaaacacatgaaaaaaaaaaaatacatgttttgttgatttctataactttggacaaagggcatacaaaatcctcctctccttggacaatgtccatccttctcatattatatttgttcgcagcaagatccataatagtctccaaatgaaaggtttaacttttggttgtgtcttttccacacaaaccaacaaacctaaacacatcctcttgttgaaatgttccaattttttttatacatttatatgttatttttatataatatattattctaggaattaacattagcattaagattatttctattagaggaaatgtgtttattgaatggtagtaattagtgcagtctttaaggttagattgatgagatctacttgttatcttcaaaatgacattgtaccattcttgatttctttgtctattttctttgtaactttcttaggtctattatggctctctccactttttcatctttttcatcctcatacacctttctccacacatggagtttgttccttattttcatgtgtgtatgcaaagacctaatgttatctttcaacaaattatcttttgtcccaacactatgcataagtgttttgtttctaagtttatcatattgttggactttcgcatcttcgtacttatttattatatttctattaaagacactcttactaatttggtccaatttgacctaaattgataacataattacacaatttttatgtttggaccatacaggatggttccattatcgtaaatgttttgttttgttcggcttaattcattagtccacttaagacctccacattcttgttttggcgttcccaatgccatattaacacacttacactagcttcctcctcactccttgtaggttgttgtgttatatctttttcctttgtactttccttagacgtagcactatggtctaaaggtttttgtgcttattgtacaattggttcttcatcgtttcctaccttgttcctcttcccgcatgttatttatcttttcctccattgtccatttcacccaattatctttagtatcataccattgtggcttagtttccacacttatcttgtttgccatagatgaaccttcaaactccattggttcataccctatggtctatcatttgcatacaatttggttatcatcctccatggtgataactccattaaagctttttttctatgagaaacttgatataggattgatgtcctaatgaatactccctcaactctaattttttttgacaatttattgtgttcttccatataaaagaatgtacatcgtattcttaccactttttttatgcatttaacactcttgctaaaaaaaggaacaccgtaaaactaatcacatactaatttattacctaataacaattatgagttttacttgtaaatattattgaacatgtccccaactatacccttttactaaaaaacatgtcatttattttattctcttgtgatccaaaaatcacaagtttaattgtccttaaaataagacaatataattcctctcattttccataaatccactccaatactcggtagtttagtttatcaaacattgttaggtctttgagtcttctcgtccaactttctgtaagccatttttcttcttccatgtctgaatttattatcatatcattacatttctcaacatggtcttgaagtctcatccttgacttccatttttgtgtagtagcttttgacttcaccactttcacatatgcaagttcatcaatcatgtgttgtattccatgttctattatactttgctttaccccttattttggtgcatgtgaagttattgtctttcaatttgtattatttgtacccttttatccttaaggatgttaaaacaaaattcatagatcttttctcaaactcaccatgtctcaagaatattttccatagatcttttcactctaagcatttgagaacttataaaacaagaaggtaaagatgttgtttcagtaattctctcaacttttcttttcgctcgacctcgctcataatcaattgctccccacgtcttttattatcttatttataatcaatgtgaaatattaaaaaaaaattaatatatcccatcatatcctataatattagtgtgaataacataattgatttaaaaatatatttaaatattttcttaaaatttgagattaaatttagtttaactccataataataacatttaaagtgtttaatactctatatctagttatcttcgtgagaatttgtatttaaaatctcataccaattaaaaataatatcaatatttataatcttccttttataaattgattttgtgagatttaattaaatttatatttatattttaagatagtgttaaaatattttctacagattgtttattgtcgggtcaaattaggtacaaaatgtttaattttgcaatctctatgctcgataaatcttatcctgaatttgagagggtgttacatatccaacttagactgctatataacaaatatattacttacaagataataattatctaataaattaatggattgaatgagctttaaacttgatctctaagattttttttgatcttactaattatttgaaacctctttattgattcatcaacaatctactcacaaaattgtagtcttgacaaatttttaatcgtgtaaaaaaatgtgataatgcttatcatatgctaatgtcaaacttgacctatactttagatatgattatcatacatctagttgttctttcatcttctttattatatctattaattgtttttctctctttatcttgcttgtttccacttatagactctaaaatgacattcagcattcgtaacgattaaaagaataagaaataataagatcaacacattattttatttttaatgaaactatttaataataaacactgcacagtggcagccgggcaagattataaatttgggaagtggtgtgctaacttaacacttcaagatttattatcccttcttacaactacttttattaatatttttaattaatatttcaattaaataacttaattcaatattttattactatatagaattattaaatggttgtaaatgcatgtttgaaaatagttttttctctaagggaaaaattattctaacaccaatttttataaatatatccatatcaaaactgtgattaaaataataaaatattaaattaagttaaaagtatttctttcttaacgttcatcttataattgtatatactactattttattgtaatatagtattttattagaaaaatcagttaaaagtaatgtatttaatgaataagatatttttccttaactaagacataaattaaattttaataaatagttattattgagttgttaggaagtggtagaagtttataaagaagttttaagtatcactcttcttttcttaattatagcttagaatttctcgtttataaagtcaaccaatatgccaaacaaactcttatctctttgcttataaagtggcttaggctgcggcaccacacaacactgcaagaagcattcacaaacaaca

SEQ ID NO: 132 - DNA, 3251 bp, Phaseolus acutifolius
  misc_feature 1043..1196: note = n is a, c, g, or t
  source 1..3251: mol_type = genomic DNA
attcctttccagcctttgtagattcccttgttcttttgttgtctaatctaatcatagtatttttgtttgggttgctacgaatttgagtctgaataggattaatacaagaaacattacagatttaggcttaagattcattgtcataccatgaagaaaatgcagctcattctatcgtgtacctttgcaacaaagataacttaattatataactttttgtgatgacaatttgtcatcaacatgttgcttttatattttcattatctgtgtaatcatgcatatggaaatgctagataatgttcttatctttcgaaacaaaacaatttgtttgctcataatttttcattacatcaacacaccattttgacttcttaacttaattgtagtcttaaggttttaatttgaaatggagcttaatatatggtgtgatccatgaaagcaatttcaaccagctgataagacttcattgttcttactgcaaacttttgattgttataagggaaacttcagtcaaaattatatatagggatcaaaattcagttctagaaatagaaaacgaaatatgagtattgatttaaaaacttcttttgtacatgaatcacattgaaaataagttataaattgaataagtcaaactcaattttaattacacatttttaaataaatgactaagtaggattttttaatttttaacagttttttttatcaattagtaactgttttttactaattaaaaacatttctaagtctccaaattaagtttaaatccaatcaacaacaaataaaaaatataaaagtttaaatctaaacaaacaggaagtgtatcgaatattatattacattttgaaaatttattctttatttacagtacaacatacatcataagtactaaataaataacacttaaaatatattaaatgttttaatttaaattgaattaaataaatttgaagtcttttatatgtattttgaatacttaatgttttttttttaaaattaggtatataatataaccattttagtttaattttcttttattacttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaataaaatttatttagagacttaagttaatattttataatttttttaagtacttaatttataaaaaataattttttgggtattttaatatatatatatatatatatatatatatatatatatatatatatatatacacgttttttctttgtccaccttcatggtttttcatgaattcaacacatagtaggatgtttcttcctacacctctatagcttcttgtggcccatgtttttttaaaatactaaaaatatcctttattttactgatattttttatttcaaaataaatttgaaatatttttcggattctataagtttatttaaaaatacattttaaactatgcaattggaagatattttcgtattataggaatttggaaaaattacataatccaaaaagatatttatgaattcaaaaatacattttggattctaaaattcaatgtaaaatccagaatgtattttttaagaataaaatagtttttctttctgacctatgggatgctagaagaagttatggagatgcaggaaaaacaacttacaaaagtaatgaaaaaatccaaatggtttaagcttaaattttgagatatttcaaaaatttgaaaaggtaatttcgaaatatatttttgaatccataaatgaattatgaatttttacattaagttattcaaaatatatttctaaatttttaaaaaaaattatgaatttataagttacatcatttaaaatgcattttcatattcaataatttggaaatgtgtctagcttgtataatttagaatatattttcatgttatacaatttcttttatcataccatctaagatgtattatgtatttttgtattatgcaatatgaaaatatatttagattgtataatctaaaataatttttgtgtatacaatttaaaaatatattttatattgttggagatcccatatcgattaaagatgagaattttttattctatataagtgagtggaaatctcatcccatgagccaattttatgaaattgagttagatttaaagaactttgtaatatggtatcagagtcatttgaaaaatattcaagattataaaaaattattatactttagattatataactcagaattttttttagttatgtaatctagaatgattttaattatacattatacaatttaaataaatttttacattatgtaaaatataatagtttacaatctataccacatttgaaatgaaattttagattaaacaacttcaaatattgacccataaataatttgaacttttctcgcaaaaaaaatatggaatgaagaaagaagttgccttgcatcaatagatttataagacaataaagtgttgcttggtgttagatatattttttttaaaaggatgtgatttcattgtagaaagcaatacataaactgtatcaatcatcacagtgtgaagtgatggaactctgttttttaatttacctctaggtgaagtaatgcatggataatttagttgcataggaaaataaatctctcaaactctatttagaattactgttcttaaaaattgtgagaaagtaatatgattcaatttagttttttaaaaatttagtattttgactctaaatcataattgtattgtgaatgatatacaacataatctactactaaaaaagtagcttctccatataaaaataaaaagaaattttaaggagaaaacgaacaagttaaaggaagaaaaattttttcttaacactctaataataaaaaaaaatcactcaacaactatcttttaaaataaaatattgttttataataaaataataaattttttagatatgaagtgatccttaccccattttctgcactcaagccaaagaatggaatcatttcttccatttttctgtttctatgaacattcttccattctcttcaaacaaacaattcctttcttcataaatttcaaaattccttgcttttcttttcatcttcatttttctcttctacattaagtacagtaaccaaccctttctatttccatatccaactaacttaacgtcatccaaactcacc

SEQ ID NO: 133 - DNA, 3000 bp, Phaseolus acutifolius
  source 1..3000: mol_type = genomic DNA
tctatcactctcggcgtgaggggggtgtgatggagatcccacatcgactagagataaggacatttcattgtatataagtgggtgcaaacctcaaccctatgagccggttttatggggttgagttaggcttaaagtccactttgtaacacatacaatatttgaatgaattggtttaatagtatatgagggtagaacaacaattgaaaagaaaatctacctctaagaatgattgtcccaccatttcctaccctaataaagaatataagagggagagacccttccaaatcaaggaatgaagggccacgagaaaggaataggaaaaaggaaaagaaagaaaaaacaaagaggaaaaaaattacaaaactaaataatcgtttaaagaaactaaagctagagatgtcctattttttaaatatcttattagaaggcactatgatgttaagtgtcccaatagaataacaatgtttttaaaatatcaaaagattgaaagtcaggatggagctcaatcttcacctagtgaagatgaaactctagattctgaataggaagctataccttctgaatgaaaattgttagtggtaagaagacttcttaaaagtatccatagaacttgaacagtttcaaagagaaaacctattccacaccaaatgtaaagtctttgtaaaaaattattatttgattatggatattgactcttgttgtaattgttgtagttttggttggtagacaagctagccttaattgttacatcatgcccaaaaccttacaaacttcaatggatcaaagatgatggtggtgtagtagttaatcaacaaatgagtatctcaatttctctaggaaattataaagataagtttaatgtgacatggtctctatggaagcttgacatattttacttggtagaccatgataatttgatacaatctatccatgatggtcttataaataaaataagtttctctcataaggacaaaaatataatcgtgtgtcctcccacaccttaacaagtgagagaggaccaaataaaaatgaaaaaaaaaaacttgaagaggagaaaagatagagaaaaaaactaagtcggaaaatctctctcacgcttaaaatagagggtgagaaggagttgagtttggaggtttgtgtccctccaaagtagttgttcaacaaaaatctttgaaaaataaaaataataagtctcttctagttgaacaacctattttccttttctattgcaaagagatacttgctacctcgaatcttgaactttgattctctattacatagggttcaacaaccttaaaataatttcttaaaataatttggtatttttttctcaagtacgttcctcatggacttctatctttaagagaatataatatgaaatagacttagccttggagcagcctactgaatagaccaacttatacgactaacctcaaagaaactaaagagatggaaaatcaagtaagtgacttgctaaaaaaggattgagtacaaaagagtctcaatccttgtgttgtaccaatattttttacccttaagaagaatgggtcatggaaattgtaggatcattaacaacatcactataaaatacacgtacccaattcttaggttagatgacatgttggacgagttacatgaaactaacattttttaaaattaatctttaagagtaattatcatgaaataagaatcaaagaaggatatgaataacaaattgcttttaagatccagtttggattgtacaaatggttaattatgcatcttgacttaaccaatactcctattacctttatgagattaattaatcatgtacttagtgattgcataagaatatttgtggttgtctactttaatgacatcttaatctatataataatttaaataagaacttaaatgaatcaggttcactccatataaattttgaattcttttactcaagttagcaatattttagaactataataaatgtgagtagaagtaacttacaactaatttctcttaattactaatctattttatttctaacttgcatccaatgaaaatttaggctagatttatgtgttatgaaaatgaaattttgaaataaaagtaaaattaaaaaactaaaatttcattctgaaacatcaaatccaaaatacaagtaatgcatctcacgattatttctttttaatagaaaacaaaatctaaaaacatattttaaaaaacaaaaatatgaaaacatatattgaaaaacctattctggaaagtctaattcaacaaaatattctaaaaactcaaatctaacaaaagccaagagaaagtaacatttaaggcaatttaatacatggactttgggaacacaaagatcagaagttgtttgtagcctataattgaagtctcttccccctacatcataaatcatagtaattccttaaccgcgtaaagttccttagattgtgtctttccttcacactaggacaacacatgccccataaattctaactgcatatcattacgccatcccttccttccactttcctattcacaaaccttctcacttcctttcttcacacaatctaattctctaaccttccaaccccaacaacataccctaatcccttcaccattcaaccttcataaaccccctcttacccatctcattcaatcatctccaatcctatcccatgcaaaatcataacccaataaaaccattctccttgtcaatttagtgtcacaataatcacactttgttgcttaaccaattctcaatacctcaattccctaggatcaaggttcacacatatcatactaaggctctgtttttttgtcttctctagagcttgtctttcttaaaaaaatatatttatttttttaatataggaatttgtagtttatgattacttttttttaaaaaagaattcttttttagcctcatacttttgcaagtagtcttctaataattaatttttctctaacactaaaataatattgcaaacatagatttagtcaacaactcagaaa

SEQ ID NO: 134 - DNA, 3283 bp, Phaseolus acutifolius
  misc_feature 941..1073: note = n is a, c, g, or t
  source 1..3283: mol_type = genomic DNA
aatgaaagaaaatacaactatgttacaaataaagaaatatcaattttgtatatactatagttacaaataaaactaaaaaaacgaatgaataataaacaagttaacttttaaaattgagaatattacatgttagaattacatttagtaaattcaaatataacctaaaatatctataaaaaaaacactaaacaaatgaaccataaatcaaattttctattataaagaataggaaatcaaactcgtaagagttcttacaaaattttaataattttagttttaagtttcttttcattacttattttaaataattataataatgataattgatatgatgttttcaatcatctcttgtctagaagttgaggataaaagaaatttaaatatatcttcttcccttaattttctaaggtgcttttgaaggataaaatcgtgatgaaactccgagttccaaaatagacaatactttaggtatagttattgaccctaacaatgttatcttaacaaacttgaggtcaaaactcttcttttccttagacttcgatgtgaggcttgttctgaaataatcgatagtcttatattacttaaaagtcaagattatgaataatttaaatatatcttcatcccttaacattttgaaatgttttttaagagaaaaaaatcatgatgaaatttcaagttctaaagtgcattatatcacaaataaatgtgattattgactctaataatgttatttcaaaaaaaaaaattatacaactataacgagtcaaactcctttctaataacaacaataatgtattatttgtgctttcatctatggttagttctaccttattagttttatgttaaggcttataatgtgaaataattcattcgtttttctacgaaactattattttcttagaaagagtttcaaaaaagtgtttaaataataataataatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcaactaatcaattatcttataaaataatcaattggtataagtaaaataattcattatttgaagttagaaaaaaatacaatatttgtggagaatctattaagattaaaagtaataaattctatcatttttattaaatttgtatttttttattaaaattaataaataaaactaatcaatttacaaatcattcttgcttatttttaattgttcttcaataactctacaagattaaacataatttttgctcaaaaatagtaaatgatatatctctttatttttttggataatctattacatcaaatgatatatttccatatttgaatttttcagtcatatttttggtgtttcgtgtaataggcatttacatgttgatctaaaaattaaaatactttaaatgtgggatagttatagccaaatattctacttggtgtgttattttacattaattaatacttataactttttaatttaaacatttcttttttaaatatatgttacttatcttctttacttattcattgaaaataattatacatatgtttatctcattatattaaatatttttatttctatttaaaattctagatatattaaaaaattactcttaaatattaaatttataccctaatttgaagggtgattagaattctccattatcataatagttgtttgtgtggaaagtatgtagtggagagcacatccttcctccatgcttctatttttgggtgttcacttcttcctctctttcattccaaaaaagaataagacacttgaaatagaaaaaaaaaataattaattttacagacataaattaaaaattatttacgttttctctaattttagaaattaattttacaacaaaatacaatgttgtgtatatgtttacaaacgtgaggtatatatcttggtagttaaaactttataattaattagatactaatttaaaaattatttaacaataatataaactaatttagaaattaaaatttttttagtttctaaaatgatctctaatttagttaatataacaattaattttttttctctaaaattaatttttattttataatttttttttagtgtatacttaattttgtatctcgtacaattcaacttaattatatttttatgggttggattgaattgttgtgtaaaattggatcccctaacatgtttataaattttgaatatgaaaaactcaacttcataataaatatataataaaatatactagtattatttgtgtgtatatatagaataagaattaccgctatttttaattattaaaaatatttcttttggaaatcgttttattaaaaaagaattattttttaaattactcaattcattaaaaatagttagatcatactaaataaacaagtaaacaaattggatgggatgcaattaaatttaattaaataacaaataaattaaactccaccttaacgtgttgagtagtatatatcatagacgagaacgaaaattcatacatgataagacttttccaaacaataatatcttttggcttgctatcaaaacttccacattcaatatgattcatatgtgtattgatccataacttgttgttaaagatgttacgttttataagttgagttaggctcttccctaacatgagtttatcttgttccaattcttcattggtgtgcacataaatctgcttttatgtggggtactggttttttatttctgcatctgaatttggtatgttcccattgagaaacttctgttttgcttcttgacatggtcacattcactcacctttcttcccaagtttcacttcctttcaatcaatcattcaatcctctttactcacacccttccacggttcattcatcagaaatttagaatcattcatactctctaacatcatcatcatcaacacaccaaaacaaaactttaacctcaacttgtactaaaagccagttcaccaaatcaataaactatggtcaactatccagcaagtatcctccaaaaatccaaaatccaaacacctctttcctactcataacaatttaataattaaccagttgttgatagagtagccgccaataatgaaaaaacaccaagtctaagtaacaactgcattgacctcatcctctccaaaacccttatatactcacaccattcaagcattcatcatcatcatcaccacaaccaaacaccacttccaccaccttcagaagcagca

SEQ ID NO: 135 - DNA, 2997 bp, Cicer arietinum
  source 1..2997: mol_type = genomic DNA
tatgtgttttactttatttttatcgaaccaatatcatttaagtcattatctcgatcataaaaatacatatatgatcattagtctactgccaaaagacgatatagtaagtcttacatttacaaaatcattagtccacctttgggaaaagtgtgcaaagaacaattactaagttttttttttatgctaaataatacgtacagatccttaacttaatttcagttaacgttttagtcttttatcttttttttttcttctcggtttggttctttattttaattttaaatgacaatttgatcttttatgttttaaaaatgtaaacaatgtaatcattttttacaaaaattcatcaaaattttcaaacaaaactcataaaattaattatcatcttcaatataatgcaaatttcatcaaactcataacttaaatctttatataaactcatatttttattcattatttgatgaatttgatgtcgttggagataaaaatatgagttgatttgaatatttgagttatgcatttgataaaatttgcattatattgaagatgataattaattttatgggttttgtttgaaaattttgatgacttttttgaatttttataaaataatagataacattgttgatattttagaacataaaagattaaattatcacttaaatttaaaataaataattaaatcaagaaagaaaaaaagataaacgactaaaagattaaatgaaattaaattaagggattgtgtgtactatttaattttttataaaaaaaaatattttaaaaaaataaattaactcatgtatacagattgtaatcaatacttttttatccaaacttgttaatttttattttatttttatgccctttgataaggatcgatgctctggtggcttcctggcatagaggttcttttgcaataaaataaaataatcaatctctcgaaataagaaattagctaataagttaaaaaataaataaaactatgcaaacaattgtttatgattaacacgctccgttgctaacgggtatgttcaaaaacgttgcctaaacacctttaaacaataatataaaatgcttttagcatattaaattcattaaaaatattcaattgagtaatttatgagggagactctgattaatattttgatatactttttcaaatataacggatgtaaaatgtatatttttctaaaaaggaaaacggatattgattgtacattttattcaaagattttcagaatatttgttttcaagtcaacctttacatgttcaacgatcttagctaaactcatattcattgatatataaaaccaatcaatttctttaataaataagaaaaaacaatcgtttcacctgaatgtgaaaaattataaactaaaaaaatgtcaaaatttaaatgattcaatattttttcattttaaatatataaattagtgaacttatatagtggaatacgtataaaaatgattgtatttttttatctttataataaaattagaattttaaaaaataaaattatgtctttttcaattttcctattcttattatttttcgatccctcaaatttatgaaaaggaaaaaaaccatatttcataaaaaattcatttttactaacaattattaaatattttattattttattaaaattactagtaattttaatttaactattgtaattgttacaaaataaatttttattgaaaataatctgattttttctattaccaaataaaaatatttaacaattactaaaataatccctcaagttaaatattttactaaataaaaattactcagattatttttcaaaaaatcttttctcgttagaatcttttcaaataaagatattttttactcagattattttcaaaaaatattttttgactcaaaagttataaatctttttcaaaaaaaaattactcagattattttccaaataaataaattattacaatttttttgtatttacaaaatttagtaaagcatttaattgagtaatttgtgaggaagactctaattaatattccgatgaaaaatattagcaaaatactattaaacacattttctaatctatcatcttttattagttaaaatttacatgagtctcataaaatgtaaatgaaatccattcaatttggtgggatctatgtaaatttcaattaataaaaaaaatatattggaatatatgtataagagaatgtatagtcaacactcttctattcccatatacttttttaaacataactgatatagattgtatatttttataaatatagtaatgatgcgacatcttttaaatgacatgatatataatatgatgatattgatggaaggaataatatctatgaatatacaataaaaacattctaaaaattttagttttaatttctaaaattatttatttttatgatttaattaataatataaatagttaatgtatcttaaatggttttatatccttaaattgtatgttatatcacctaaaatatattaaatctccatttttaatttaaaaagtcaaaaactatattaaaattaataaattttaaaatagaagaacaaattcaacgaattaataaaagttgactataattcttaaaaaaaaaaaaatcaatgtagattgcatattgtctcaaaaaatttaaaaatatttgttttaaagtcaacgtttaaatgttaaacaatcttacctaattaattcatattaaaaaactaacttaaattagaattttaatttcaaattagaattaattttaaaaaaaaaaggtaaaaaagaaagatatcaaaaatcaatttcaaatctgtaaaattgattttggggtgcttcaaacaaaaagcaaacaagcacattgtaaaaagtctcatcaaccaaacacaaaccaaacacaaattaattgatatataaaaagagaggaaaggaaaaacaagcaccacacacaacacaactattgcaacaactctcatcaaccaaaaacaaaaaacacaaacattgtaaca

SEQ ID NO: 136 - DNA, 1769 bp, Cicer arietinum
  source 1..1769: mol_type = genomic DNA
tggaaattcaatgttcatggtttgctcattattatttccaatgttaaatggtttctatatatttaaaaagaaaataagaaacaactgattcaattgcttaatgaatttctgttaagaataaatctttctatagaacacaaatttaaattttaattgaaataattattgattaaattttatttatctcttaaatgaattctgaattactaaagtttcattacaatggaaactagaggttaaaaaaaaaaaaaaaaaagtaataaatttttaataactcttgcattgtattgtagatttttaataaaatctattgtgtaaacttgtaaatttattttatgttgctgtacctattgttgatagaaaatccaaaaagtaaaattccatttacaaacttggttgttctaacatcagcgacttcaatccaattacttatcatacaacaacaagacaaagcaaacaatgacaaaggcctcaaaaaaaaaaatacttaattgaaagaattcaaaaaaaaaaaaaattaatgagtaatcagagttttgtgtattgataagtaattagtgatagattgaagttgaaattacaaaatgtacggaataattgtacctaattggataaataagtgaaaaagtttgtggtatatattagtaaaacaaaatagaataaaagtagaagatagagaaaaataaaagatacaaaaaatagtatgctttagaagataccatattttattttaatattataataatagataattaatttgaacaataattcatctcaagtttataatgagttattaaacatagagtaaaatttgatatgtgtgtgttatatgctctcttcaaattgaatgcttgcacttgcactatggtgatggtgagcatgaaacatttttaatactttttatagttggcacaatggtgtgctaattatattcaaacctattgatatattcttatatatactttatccaatttaatataatctattttcaattcaacaattatgtgttgtcatgtgtcacattttcttttttcttaattcagcataatactaagtatatacaataattaatttctccatggaataccagaaaagcaagtacattattctaaggttcctttctaacaaaatcatggtttttacttgttttgtaactttcatgtataaggacttatgttgatcgtgtaatttggtcacattcatgtaccttaccttaaaattaaatcaatcattcaattctctttacttccactctttacacaccttgtaatctcatttaagccgccataaaaaacaaaccttcccatcaacttgtgctataatattagtttacaatttcaatattatatagtcaacttgacaaccatatttaatcatgcatacatcaaccaaattactcaaatcccattattctcctattcatgacaattttataatatatgaagcaacttgtagatatatagagtagttgccaatattgataataatatatccggtgtatatattatcgatatttcatattaatgacatatttaatgtaagatatgtctatttggtatctgacaccaacactataatactgtttcatttttttaaattattactaatgtctatattcagtaccgtgtctgatgtcgaagtatgtgtactaaagaaaagaaagaactccactcagctcaacacataccctcaaagaaacactataaaactcatgattcaatatcactacaatatccattaagaccttaatcatcataatcaaacacaac

SEQ ID NO: 137 - DNA, 29 bp, synthetic construct
  misc_feature 1..29: note = GPB1gF3 Primer
  source 1..29: mol_type = other DNA
taaggagaataagtaagtagcccttatca

SEQ ID NO: 138 - DNA, 20 bp, synthetic construct
  misc_feature 1..20: note = GBP1gR2 Primer
  source 1..20: mol_type = other DNA
agaaggagcccaccaaagtt

SEQ ID NO: 139 - DNA, 20 bp, synthetic construct
  misc_feature 1..20: note = tnt1-R Primer
  source 1..20: mol_type = other DNA
cagtgaacgagcagaacctg

SEQ ID NO: 140 - DNA, 20 bp, synthetic construct
  misc_feature 1..20: note = tnt1-F Primer
  source 1..20: mol_type = other DNA
acagtgctacctcctctgga

SEQ ID NO: 141 - DNA, 23 bp, synthetic construct
  misc_feature 1..23: note = GBP1qF Primer
  source 1..23: mol_type = other DNA
aaatcaatatgtttgggtcatgc

SEQ ID NO: 142 - DNA, 20 bp, synthetic construct
  misc_feature 1..20: note = GBP1qR Primer
  source 1..20: mol_type = other DNA
ttgtcggccacatatccttg

SEQ ID NO: 143 - DNA, 26 bp, synthetic construct
  misc_feature 1..26: note = GBP1clF Primer
  source 1..26: mol_type = other DNA
atgtcttcatcatcttctcttccttt

SEQ ID NO: 144 - DNA, 20 bp, synthetic construct
  misc_feature 1..20: note = GBP1clR Primer
  source 1..20: mol_type = other DNA
tcatctgctatggatccacc